SEQUENCE LISTING (45 sequences)

SEQ ID NO: 1 - protein, 12 aa, synthetic construct
  REGION 1..12: note = Synthetic
RYYRWKKKKKKK

SEQ ID NO: 2 - protein, 17 aa, synthetic construct
  REGION 1..17: note = Synthetic
FGGFTGARKSARKLANQ

SEQ ID NO: 3 - protein, 23 aa, synthetic construct
  REGION 1..23: note = Synthetic
REPRILSEEEQEMFRDFDYIADW

SEQ ID NO: 4 - protein, 14 aa, synthetic construct
  REGION 1..14: note = Synthetic
GIYRTDILRTEEGN

SEQ ID NO: 5 - protein, 14 aa, synthetic construct
  REGION 1..14: note = Synthetic
GIYRTELLKAEEAN

SEQ ID NO: 6 - protein, 14 aa, synthetic construct
  REGION 1..14: note = Synthetic
GIYRLELLKAEEAN

SEQ ID NO: 7 - protein, 9 aa, synthetic construct
  REGION 1..9: note = Synthetic
GIYRLELLK

SEQ ID NO: 8 - protein, 14 aa, synthetic construct
  REGION 1..14: note = Synthetic
AIYRLELLKAEEAN

SEQ ID NO: 9 - protein, 9 aa, synthetic construct
  REGION 1..9: note = Synthetic
GIYRLELLK

SEQ ID NO: 10 - protein, 14 aa, synthetic construct
  REGION 1..14: note = Synthetic
GIYRLELLKAEAAA

SEQ ID NO: 11 - protein, 14 aa, synthetic construct
  REGION 1..14: note = Synthetic
GIYRLELLKAEEAN

SEQ ID NO: 12 - protein, 14 aa, synthetic construct
  REGION 1..14: note = Synthetic
GIYRLELLKAEAAA

SEQ ID NO: 13 - protein, 16 aa, synthetic construct
  REGION 1..16: note = Synthetic
FIGRLCTEILKLREGN

SEQ ID NO: 14 - protein, 16 aa, synthetic construct
  REGION 1..16: note = Synthetic
LAWRLRELERELARLC

SEQ ID NO: 15 - protein, 16 aa, synthetic construct
  REGION 1..16: note = Synthetic
WIGRLCTEILRLRNGN

SEQ ID NO: 16 - protein, 16 aa, synthetic construct
  REGION 1..16: note = Synthetic
LAWRLRELERELARLC

SEQ ID NO: 17 - protein, 16 aa, synthetic construct
  REGION 1..16: note = Synthetic
AIGRLCTEILRLRNGA

SEQ ID NO: 18 - protein, 16 aa, synthetic construct
  REGION 1..16: note = Synthetic
LAWRLRELERELARLC

SEQ ID NO: 19 - protein, 16 aa, synthetic construct
  REGION 1..16: note = Synthetic
WIGRLCTEILRLRNGN

SEQ ID NO: 20 - protein, 16 aa, synthetic construct
  REGION 1..16: note = Synthetic
LAWALRELERELARLC

SEQ ID NO: 21 - protein, 16 aa, synthetic construct
  SITE 10: note = X is L-homoarginine
  REGION 1..16: note = Synthetic
WIGRLCTEIXRLRNGN

SEQ ID NO: 22 - protein, 16 aa, synthetic construct
  REGION 1..16: note = Synthetic
LAWRLRELERELARLC

SEQ ID NO: 23 - protein, 16 aa, synthetic construct
  REGION 1..16: note = Synthetic
WIGRLCTEIRRLRNGN

SEQ ID NO: 24 - protein, 16 aa, synthetic construct
  REGION 1..16: note = Synthetic
LAWRLRELERELARLC

SEQ ID NO: 25 - protein, 16 aa, synthetic construct
  REGION 1..16: note = Synthetic
WIGRLCTEILRLRNGN

SEQ ID NO: 26 - protein, 16 aa, synthetic construct
  REGION 1..16: note = Synthetic
LAWRLRELERELARLC

SEQ ID NO: 27 - protein, 16 aa, synthetic construct
  REGION 1..16: note = Synthetic
FIGRLCTEILKLREGN

SEQ ID NO: 28 - protein, 17 aa, synthetic construct
  REGION 1..17: note = Synthetic
  SITE 1: note = X is L-beta-alanine
XLAWRLRELERELARLC

SEQ ID NO: 29 - protein, 16 aa, synthetic construct
  REGION 1..16: note = Synthetic
WIGRLCTEILRLRNGN

SEQ ID NO: 30 - protein, 17 aa, synthetic construct
  SITE 1: note = X is L-beta-alanine
  REGION 1..17: note = Synthetic
XLAWRLRELERELARLC

SEQ ID NO: 31 - protein, 16 aa, synthetic construct
  REGION 1..16: note = Synthetic
AIGRLCTEILRLRNGA

SEQ ID NO: 32 - protein, 17 aa, synthetic construct
  SITE 1: note = X is L-beta-alanine
  REGION 1..17: note = Synthetic
XLAWRLRELERELARLC

SEQ ID NO: 33 - protein, 16 aa, synthetic construct
  REGION 1..16: note = Synthetic
WIGRLCTEILRLRNGN

SEQ ID NO: 34 - protein, 17 aa, synthetic construct
  REGION 1..17: note = Synthetic
  SITE 1: note = X is L-beta-alanine
XLAWALRELERELARLC

SEQ ID NO: 35 - protein, 16 aa, synthetic construct
  REGION 1..16: note = Synthetic
  SITE 10: note = X is L-homoarginine
WIGRLCTEIXRLRNGN

SEQ ID NO: 36 - protein, 17 aa, synthetic construct
  SITE 1: note = X is L-beta-alanine
  REGION 1..17: note = Synthetic
XLAWRLRELERELARLC

SEQ ID NO: 37 - protein, 16 aa, synthetic construct
  REGION 1..16: note = Synthetic
WIGRLCTEIRRLRNGN

SEQ ID NO: 38 - protein, 17 aa, synthetic construct
  SITE 1: note = X is L-beta-alanine
  REGION 1..17: note = Synthetic
XLAWRLRELERELARLC

SEQ ID NO: 39 - protein, 16 aa, synthetic construct
  REGION 1..16: note = Synthetic
  SITE 10: note = X is L-homoarginine
WIGRLCTEIXRLRNGN

SEQ ID NO: 40 - protein, 17 aa, synthetic construct
  REGION 1..17: note = Synthetic
GLAWRLRELERELARLC

SEQ ID NO: 41 - protein, 10 aa, synthetic construct
  REGION 1..10: note = Synthetic
WIGRLCTEIK

SEQ ID NO: 42 - protein, 6 aa, synthetic construct
  REGION 1..6: note = Synthetic
RLRNGN

SEQ ID NO: 43 - protein, 16 aa, synthetic construct
  REGION 1..16: note = Synthetic
LAWRLRELERELARLC

SEQ ID NO: 44 - protein, 5 aa, synthetic construct
  REGION 1..5: note = Synthetic
APRPG

SEQ ID NO: 45 - protein, 8 aa, synthetic construct
  REGION 1..8: note = Synthetic
GPLGIAGQ

(19) United States
(12) Patent Application Publication
(10) Pub. No.: US 2026/0174880 A1
(43) Pub. Date: Jun. 25, 2026

(54) ELECTROPHILIC REACTIVE LINKERS FOR LABELING OF POLYPEPTIDES AND METHODS OF USE THEREOF

(71) Applicants: The Brigham and Women's Hospital, Inc., Boston, MA (US); The Broad Institute, Inc., Cambridge, MA (US); President and Fellows of Harvard College, Cambridge, MA (US)

(72) Inventors: Amit Choudhary, Boston, MA (US); Shaimaa Sindi, Cambridge, MA (US); Mahilet Tefera, Cambridge, MA (US); Jeffrey Fung, Cambridge, MA (US); Endri Karaj, Cambridge, MA (US); Santosh Chaudhary, Cambridge, MA (US)

(21) Appl. No.: 19/552,338

(22) Filed: Feb. 27, 2026

Related U.S. Application Data
(63) Continuation of application No. PCT/US2024/044175, filed on Aug. 28, 2024.
(60) Provisional application No. 63/535,016, filed on Aug. 28, 2023.
(60) Provisional application No. 63/623,584, filed on Jan. 22, 2024.

(51) Int. Cl. A61K 47/55

(52) U.S. Cl. CPC A61K 47/55

(57) ABSTRACT

The present disclosure relates to electrophilic reactive linkers, which find utility labeling target substrates according to the formula L₁-El, El-L₁, or L₁-El-L₂, wherein El is an electrophilic reactive group and wherein L₁ and L₂ are linking molecules. Molecules according to the present invention find use, for example, in multifunctional chimeric molecules (e.g., bifunctional molecules), which make substrate modifications such as post-translational modifications to targets that are not the natural substrate; accordingly, diseases or disorders may be treated or prevented with molecules of the present disclosure.

### CROSS-REFERENCE TO RELATED APPLICATIONS

This application is a continuation application of International Patent Application No.: PCT/US2024/044175, filed Aug. 28, 2024, which claims the benefit of U.S. Provisional Application No. 63/535,016, filed Aug. 28, 2023 and U.S. Provisional Application No. 63/623,584 filed Jan. 22, 2024. The entire contents of the above-identified applications are hereby fully incorporated herein by reference.

### STATEMENT REGARDING FEDERALLY SPONSORED RESEARCH

This invention was made with government support under Grant No. (s) N66001-17-2-4055 and HR00112120010 awarded by the Department of Defense and Grant No. (s) GM137606 awarded by the National Institutes of Health. The government has certain rights in the invention.

### TECHNICAL FIELD

The subject matter disclosed herein is generally directed to functional chemical conjugation molecules for labelling substrates.

### REFERENCE TO AN ELECTRONIC SEQUENCE LISTING

The contents of the electronic sequence listing (“BROD-5870US_ST26.xml”; Size is 58,536 bytes and it was created on Feb. 20, 2026) is herein incorporated by reference in its entirety.

### BACKGROUND

Multifunctional chimeric molecules (e.g., bifunctional molecules) utilizing post-translational modifications to perform neo-protein-protein interactions typically rely on allosteric or active site inhibitors to bring together two substrates because they are abundant and, in most cases, have been shown to be safe in vivo. Electrophilic reactive linkers described herein allow multifunctional chimeric molecules to label desired polypeptides regardless of the binding moiety. Covalency of a multifunctional chimeric molecule to the desired polypeptide can increase binding potency, prolong ternary complex formation, and expand the applications of bifunctional molecules.

Citation or identification of any document in this application is not an admission that such a document is available as prior art to the present invention.

### SUMMARY

In one aspect, the present disclosure provides an electrophilic reactive linker according to the formula L₁-El, El-L₁, or L₁-El-L₂, wherein El is an electrophilic reactive group and wherein L₁ and L₂ are linking molecules. In an example embodiment, the El reacts with a nucleophilic reactive group. In an example embodiment, the El is configured to facilitate attachment of the electrophilic reactive group and all or a portion of L₁ or L₂ to a Cysteine, Serine, Threonine, Tyrosine, Glutamic Acid, Aspartic Acid, Lysine, Arginine, Histidine, or a Methionine amino acid on a polypeptide.

In an example embodiment, the linking molecules L₁ and L₂ are independently selected from alkane, alkene, amine, either, thiol, sulfone, carbonyl, acyl, ketone, carboxylate ester, amide, enone, anhydride, imide, PEG, or any combination thereof. In an example embodiment, the linking molecules L₁ and/or L₂ comprise rigid molecules.

In an example embodiment, the electrophilic reactive group is selected from N-acyl-N-alkyl sulfonamide (NASA), dibromophenyl benzoate, or N-sulfonyl pyridone. In an example embodiment, the El is a photo-reactive group.

In an example embodiment, the electrophilic reactive group is selected from the group consisting of:

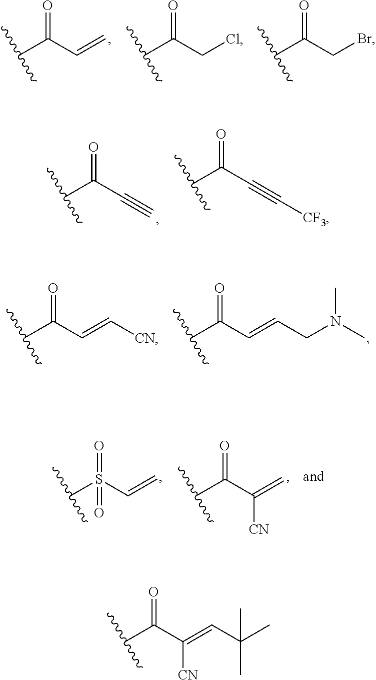

In an example embodiment, the electrophilic reactive group has the formula:

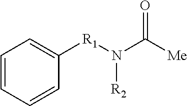

wherein R₁ is selected from C—O, SO₂, Me-C—O, or Me-SO₂, R₂ is selected from H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; an aliphatic halide such as —OCF₂Cl or any combination thereof, and the benzene ring is optionally substituted at any position.

In an example embodiment, the electrophilic reactive group is selected from the group consisting of:

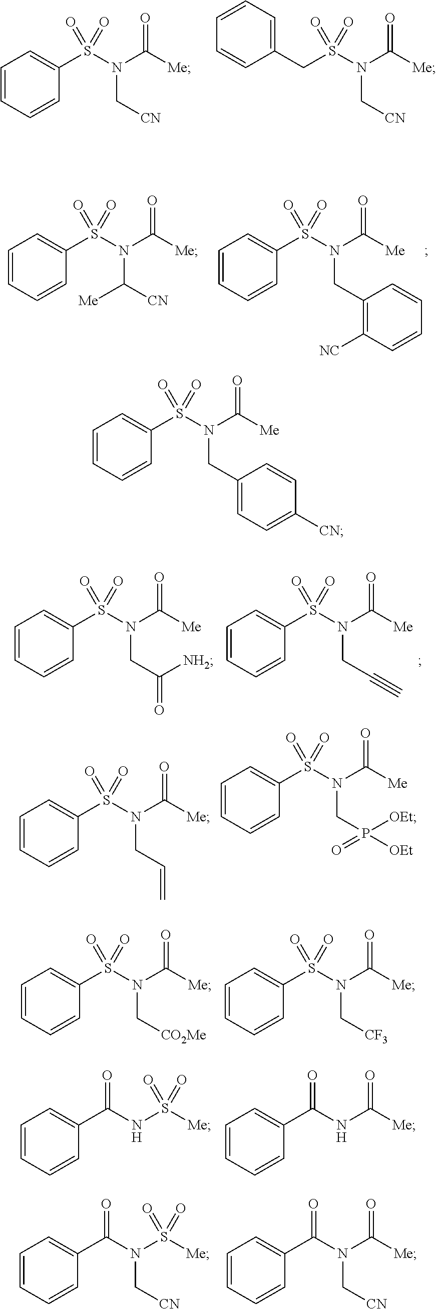

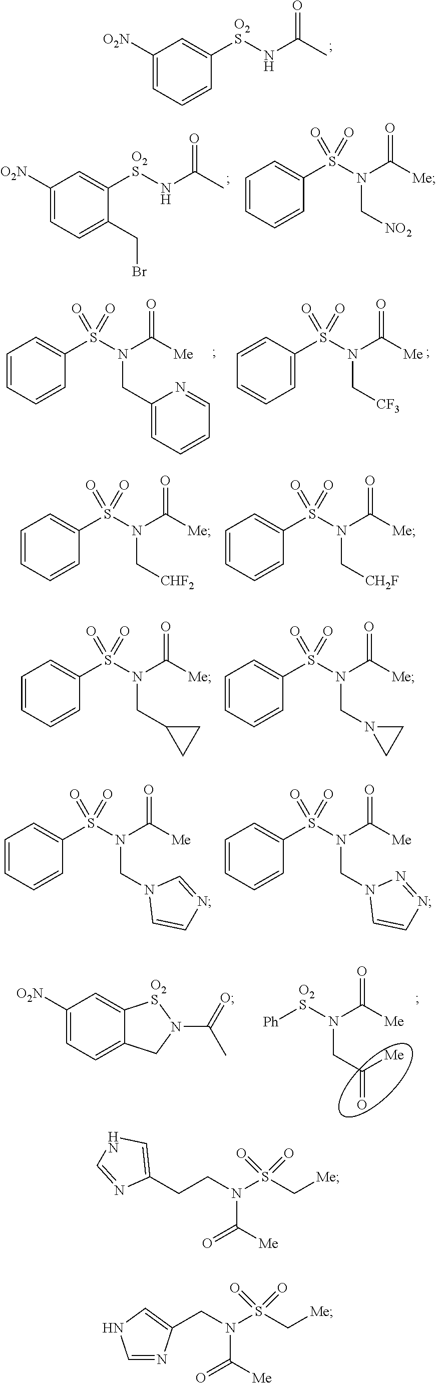

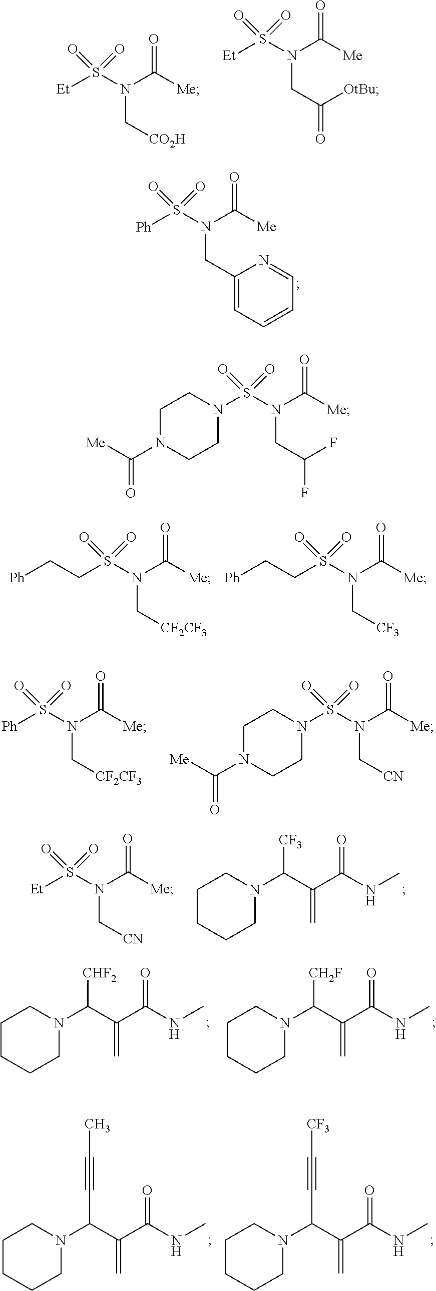

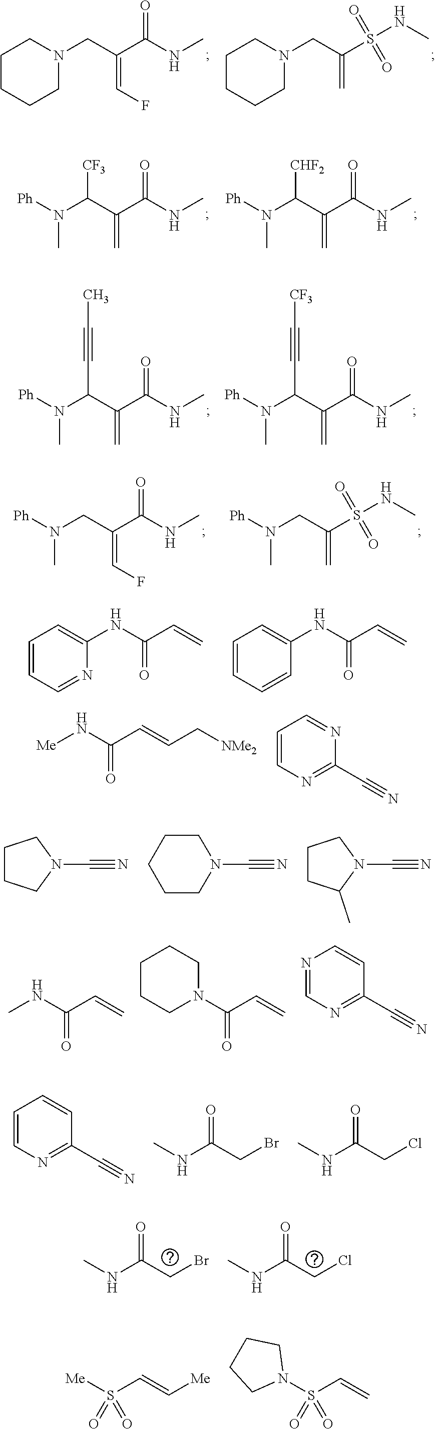

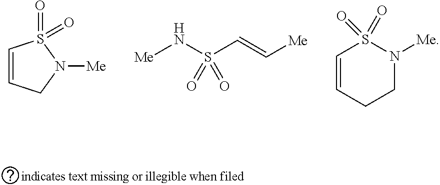

In an example embodiment, the El is configured to facilitate attachment of all or a portion of L₁ or L₂ to a cysteine, and wherein the El is selected from the group consisting of:

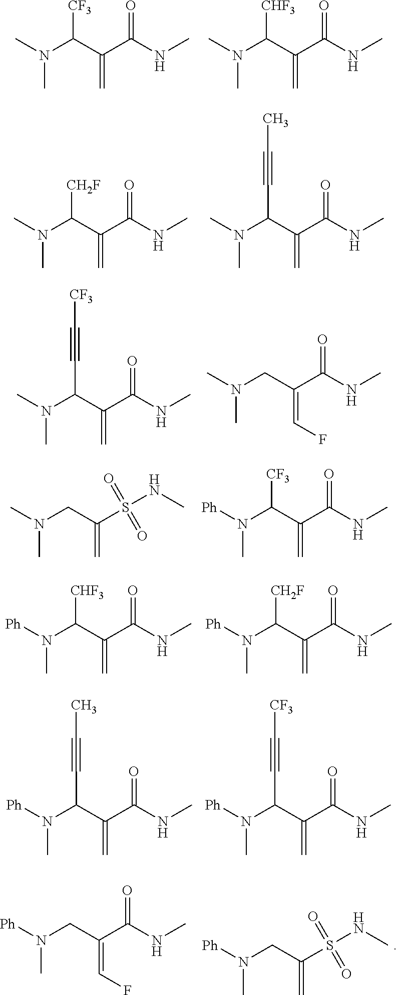

In an example embodiment, the El is configured to facilitate attachment of all or a portion of L₁ or L₂ to a lysine, and wherein the El is

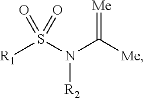

wherein R₁ is selected from the group consisting of;

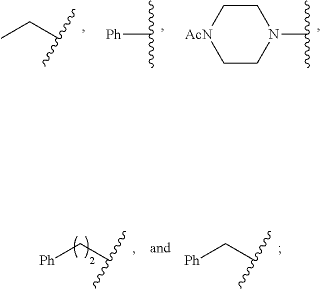

and
wherein R₂ is selected from the group consisting of

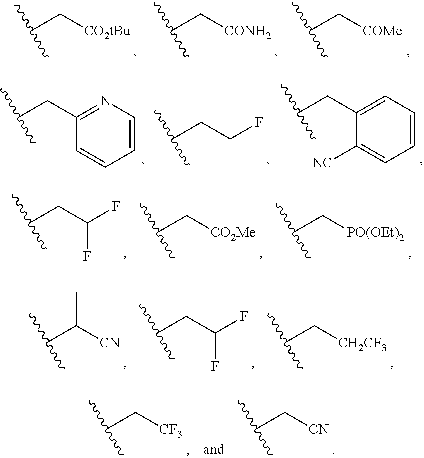

In an example embodiment, the El is configured to facilitate attachment of all or a portion of L₁ or L₂ to a lysine, and wherein the El is selected from the group consisting of:

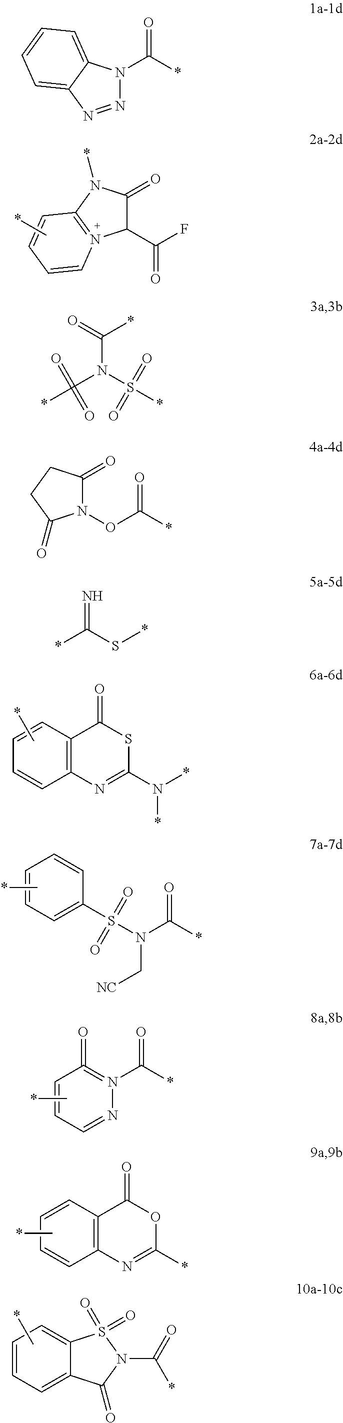

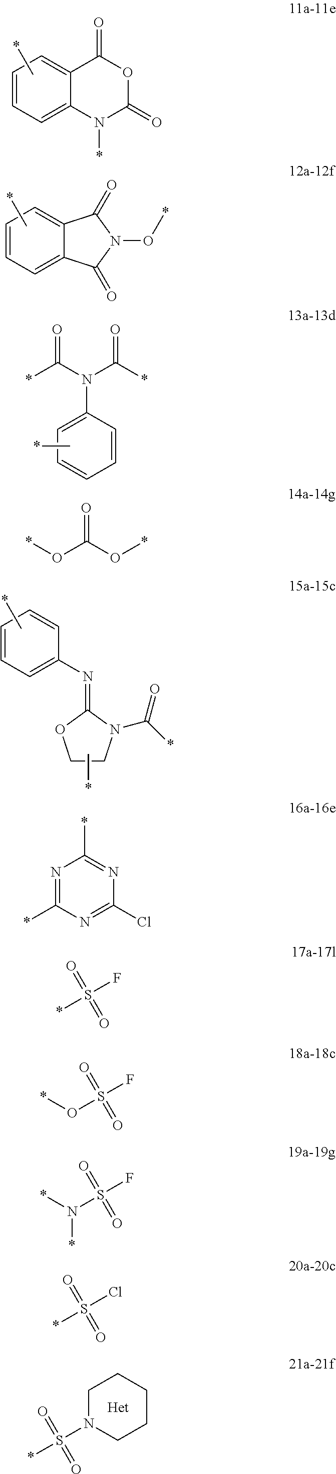

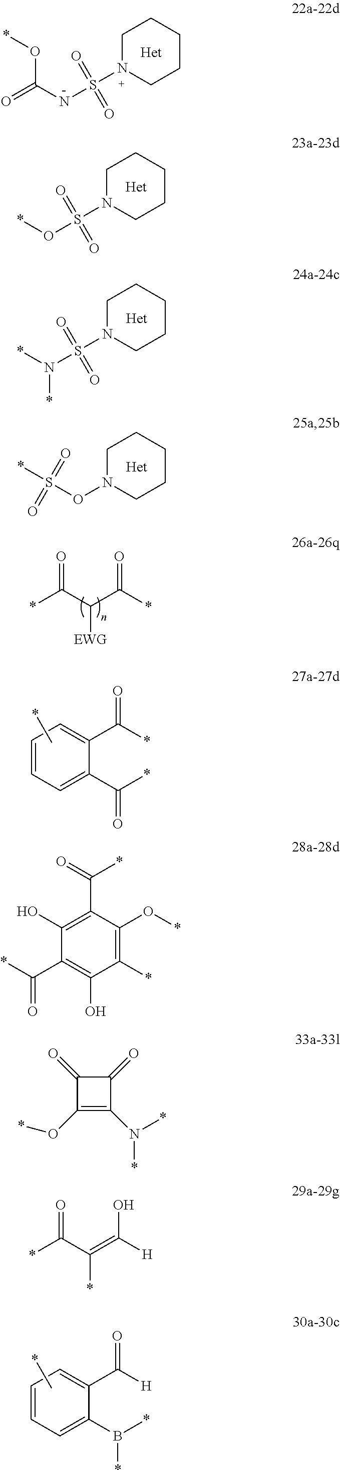

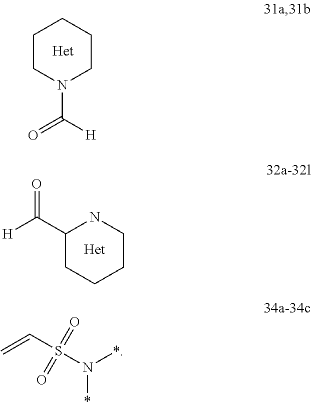

In an example embodiment, the El is configured to facilitate attachment of all or a portion of L₁ or L₂ to a methionine, and wherein the El is selected from the group consisting of:

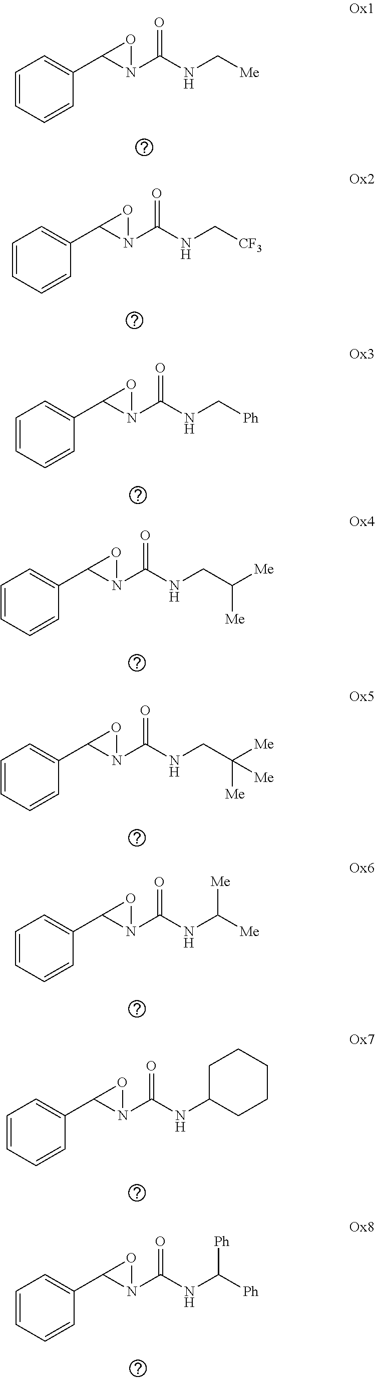

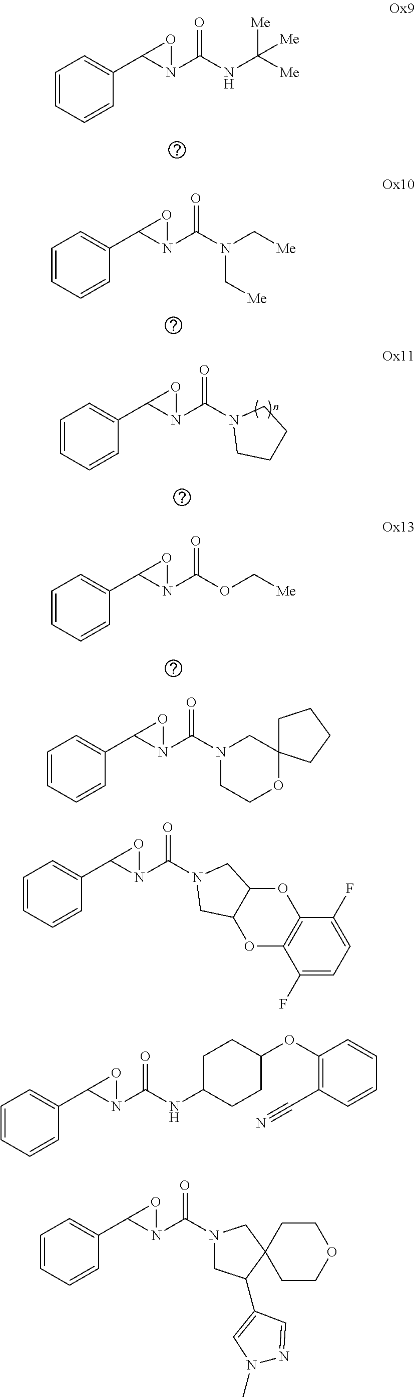

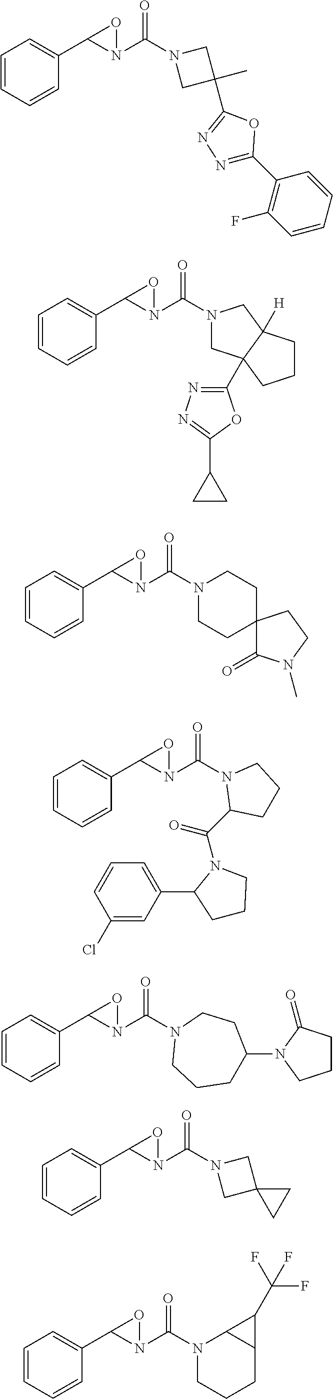

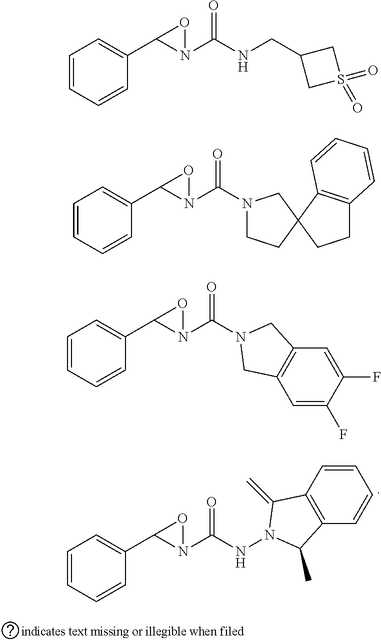

In example embodiments, the electrophilic reactive group is

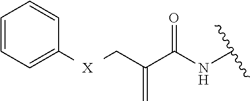

wherein X is selected from the group consisting of:

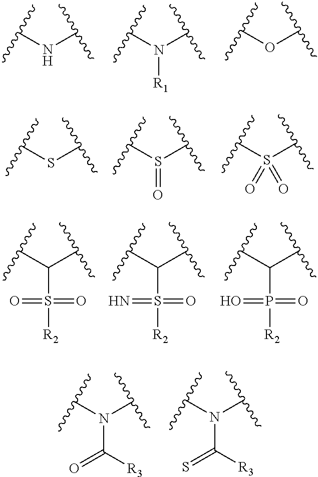

wherein R1, R2, R3 individually comprise of an alkyl group, aryl group, or a heteroatom optionally O, N, or S.

In example embodiments, the electrophilic reactive group is

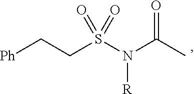

wherein R is selected from the group consisting of:

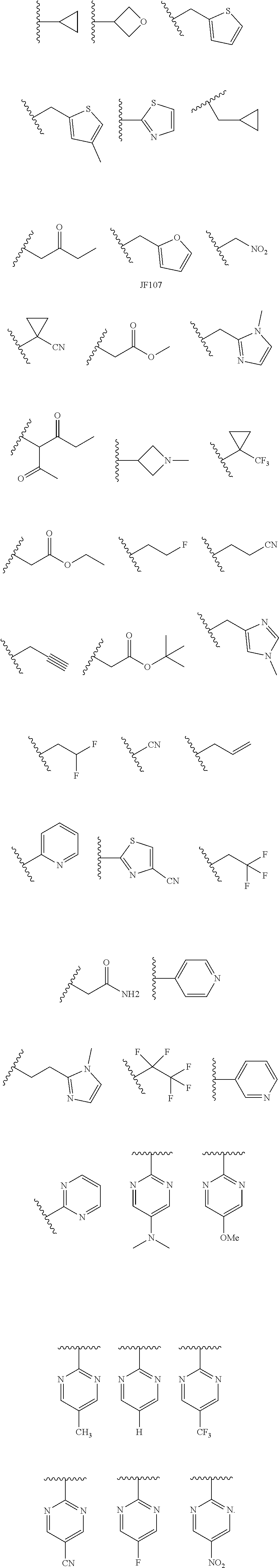

In example embodiments, the electrophilic reactive group is

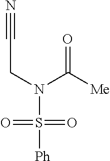 [SMILES: CC(=O)N(CC#N)S(=O)(=O)c1ccccc1]

or an analog thereof.

In example embodiments, the electrophilic reactive group is

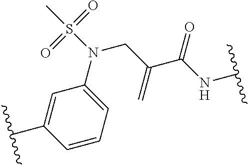 [SMILES: *NC(=O)C(=C)CN(c1cccc(*)c1)S(C)(=O)=O]

or an analog thereof.

In example embodiments, the electrophilic reactive group is

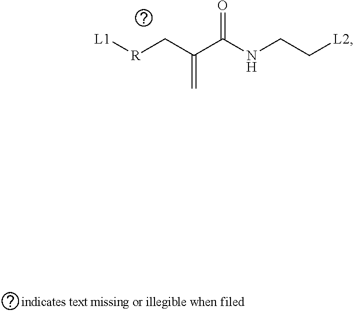

wherein R is selected from the group consisting of:

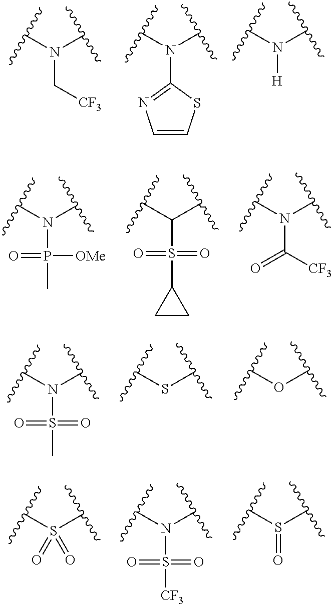

In an example embodiment, the electrophilic reactive group linker further comprises a bio-orthogonal group. In an example embodiment, the bio-orthogonal group is selected from tetrazines, triazines, cyclooctenes, cyclopropenes and diazo. In an example embodiment, the bio-orthogonal group is selected from the group consisting of:

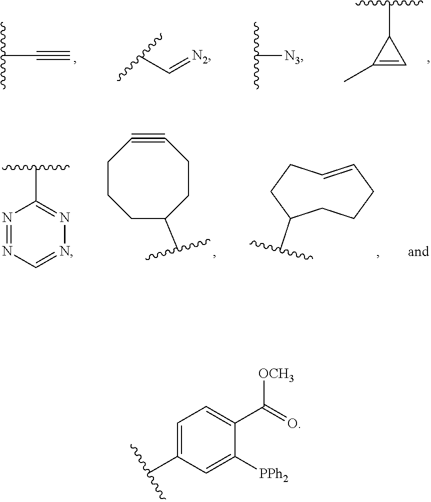

In an example embodiment, the electrophilic reactive linker is capable of covalently labeling a target polypeptide. In an example embodiment, the labeling comprises covalently bonding to a nucleophile disposed on the target polypeptide. In an example embodiment, the electrophilic reactive linker further comprises a target binding moiety connected to L₁ or L₂ and configured to allow the El to covalently attach to the target polypeptide. In an example embodiment, the electrophilic reactive linker further comprises a modifying polypeptide binding moiety located on a side of L₁ or L₂ opposite the El and capable of binding a modifying polypeptide. In an example embodiment, the El reversibly bonds to the target polypeptide.

In an example embodiment, the electrophilic reactive group linker further comprises an orienting adaptor. In an example embodiment, the orienting adaptor is selected from the group consisting of:

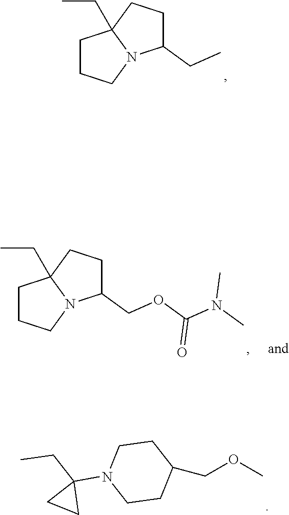

In an example embodiment, the modifying polypeptide is a phosphatase, a ubiquitinase, a deubiquitinase, an acetyltransferase, deactylase, methyltransferase, demethylase, or glycosyltransferase and the modifying polypeptide binding moiety binds a neo-substrate for the phosphatase, the ubiquitinase, the deubiquitinase, the acetyltransferase, the deactylase, the methyltransferase, the demethylase, or the glycosyltransferase.

In one aspect, the present disclosure provides a bifunctional molecule comprising the electrophilic reactive linker and a target polypeptide binding moiety attached to the linker on one end and a modifying polypeptide binding moiety attached to the linker on the opposite end. In an example embodiment, the modifying polypeptide binding moiety binds to a phosphatase, a ubiquitinase, a deubiquitinase, an acetyltransferase, deactylase, methyltransferase, demethylase, or glycosyltransferase. In an example embodiment, the the target polypeptide binding moiety binds to a polypeptide to which the modifying moiety is to be attached and modified by the modifying moiety. In an example embodiment, the electrophilic reactive linker connects the functional moieties of a proteolysis targeting chimeras (PROTAC), deubiquitinase-targeting chimeras (DUBTACs), Pho-repressive complex (PhoRC), dephosphorylation targeting chimera (DEPTAC), phosphorylation targeting chimeras (PhosTAC), acetylation tagging system (AceTAG), regulated induced proximity targeting chimeras (RIPTAC), Transcriptional/Epigenetic Chemical Inducers of Proximity (TCIP), Autophagy-Targeting Chimera (AUTAC), Lysosome Targeting Chimeras (LYTAC), and or immune cell recruiting chimeras.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Exemplary chimeras for recruitment of Pseudomonas aeruginosa to antibodies, complement or macrophages and for recruitment of Mycobacterium tuberculosis (M.tb) proteins to host kinases.

FIG. 2—Binder discovery platforms for microbial targets and host targets.

FIG. 3—Selected inhibitors for covalent labeling of kinases with their residence times. Sites of the linker and bio-orthogonal group attachments are shown by arrow and star (*), respectively.

FIG. 4A-4C—(A) Representative example of chimeric small molecule designed for proximity-induced labeling of MAPK p38α based on its inhibitor SB203580 and mechanism of covalent modification. (B) exemplary deactivation of inhibitor via click reaction with bulky cyclooctyne, bulky group makes inhibitor not bind to the kinase. C. Exemplary embodiment of Sorafetinib-based chimeric small molecule designed for proximity-induced labeling of MAPK p38α with binder of PtpA.

FIG. 5—Modular components for exemplary chimeras: known binders (blue) to microbial targets and known binders (green) to host targets. Applicants used these to optimize the assays. Applicants will also use these building blocks to create pseudo-chimeras, which consist of a known binder (shown here) and a binder identified from the screen.

FIG. 6—Exemplary platform of target labeling chimeras.

FIG. 7—General strategies for preparation of NASA and its analogs/alternatives.

FIG. 8—Additional exemplary NASA analogs/alternatives according to and for use in embodiments of the invention described herein.

FIG. 9—Example kinase binding moieties according to and for use in embodiments of the invention described herein.

FIG. 10A-10B—10A) Example mechanism of embodiments described herein. 10B)

FIG. 11—Additional example kinase binding moieties with example linker/electrophilic reactive group according to and for use in embodiments of the invention described herein.

FIG. 12—Experimental validation of electrophilic reactive group addition-elimination reaction, as described in embodiments herein, with glutathione.

FIG. 13—Considerations for electrophilic reactive group design, including example electrophilic reactive groups according to and for use in embodiments of the invention described herein.

FIG. 14—Experimental reactivity validation of aryl and alkyl design considerations for electrophilic reactive groups.

FIG. 15—Experimental reactivity/hydrolysis validation of nitrile and ketone design considerations for electrophilic reactive groups.

FIG. 16—Experimental reactivity of example electrophilic reactive groups. These example results demonstrate tunability of electrophilic reactive groups according to and for use in embodiments of the invention described herein.

FIG. 17—Example design of chimeric small molecules with kinase binding moieties and electrophilic reactive groups.

FIG. 18—Experimental validation of example chimeric small molecule demonstrating phosphorylation of BTK with ABL and reduced EC50 of the example chimeric small molecule as compared to Ibrutinib.

FIG. 19—Proof-of-concept phosphorylation on BRD4 by EGFR PHICS (HEK293T cells)

FIG. 20—Cellular target engagement: Biotin Click/Cy5 azide Click, HEK293T for compound 1

FIG. 21—Cellular target engagement: Biotin Click/Cy5 azide Click, HEK293T for compound 2

FIG. 22—Cellular target engagement: Biotin Click/Cy5 azide Click, HEK293T for compound 3

FIG. 23—Induction of BRD4 phosphorylation by JAK3 PHICS (HEK293T cells)

FIG. 24A-24C—FGFR4/Pan-FGFR biochemical target engagement: 24A) In-vitro labelling LC-MS (Intact mass for addition and elimination on kinase) 24B) partial chimeric small molecules 24C) In-gel fluorescence (Cu2+ clicked fluorescence of azide-Cy-5).

FIG. 25A-25C—ITK biochemical target engagement: 24A) In-vitro labelling LC-MS (Intact mass for addition and elimination on kinase) 24B) partial chimeric small molecules 24C) In-gel fluorescence (Cu2+ clicked fluorescence of azide-Cy-5).

FIG. 26A-26B—EGFR biochemical target engagement: 24A) partial chimeric small molecules 24B) In-gel fluorescence (Cu2+ clicked fluorescence of azide-Cy-5).

FIG. 27A-27B—FGFR and EGFR biochemical target engagement: 24A) partial FGFR chimeric small molecules and in-gel fluorescence (Cu2+ clicked fluorescence of azide-Cy-5) 24B) partial EGFR chimeric small molecules and in-gel fluorescence (Cu2+ clicked fluorescence of azide-Cy-5).

FIG. 28—JAK3 and ITK biochemical target engagement: 24A) partial JAK3 chimeric small molecule and in-gel fluorescence (Cu2+ clicked fluorescence of azide-Cy-5) 24B) partial ITK chimeric small molecules and in-gel fluorescence (Cu2+ clicked fluorescence of azide-Cy-5).

FIG. 29—FGFR4 cellular target engagement: Nano Bret based luminescence with various partial chimeric small molecules at various concentrations.

FIG. 30—Proof-of-concept phosphorylation on BRD4 with various FGFR4 small chimeric molecule.

FIG. 31—Experimental results for ITK-BRD4 small chimeric molecules.

The figures herein are for illustrative purposes only and are not necessarily drawn to scale.

FIG. 32—Provides example lysine reactive electrophilic groups for use in example electrophilically reactive linkers disclosed herein, and the tuneability of said linker molecules via modifications to the electrophilically reactive group. Reactivity: 4 mL of 10 mM compound, 32 mL 1×PBS+4 mL 50 mM lysine.

FIG. 33A-33B—33A) 1^{st}-generation PHICS that uses kinase binders that are allosteric, non-inhibitory, and rare. 33B) 2^{nd} generation PHICS that uses kinase binders that can be inhibitory, allosteric/active-site directed, and abundantly available. AH may be Cys, Lys, Tyr, or Met.

FIG. 34A-34D—34A) ABL PHICS induced phosphorylation of EGFR receptor triggering signal transduction. 34B) Structure of PHICS 1.1 and ABL kinase binder 1.2. 34C-34D) PHICS-induced EGFR phosphorylation 34C) and induction of downstream signaling as observed using a luciferase-based reporter assay 34D).

FIG. 35A-35F—35A) Homo-PHICS induced neophosphorylation inhibits BCR-ABL by disrupting ATP pocket. 35B-C) Tyr253 phosphorylation (present on the P loop of the ATP pocket) is observed in homo-PHICS treated cells (or enzyme in vitro) but not in control treatments. 35C-35D) Homo-PHICS induced death of CML cells with EC50≈52 nM and those with gatekeeper mutation that is resistant to Imatinib. 35E) KRAS neophosphorylation may disrupt membrane/GTP binding and furnish neoantigens. 35F) Example of neo-phosphorylation interfering with the GTP binding/membrane translocation of KRAS.

FIG. 36A-36G—36A) A PHICS from BTK inhibitor for target protein BRD4. 36B) Structures of BTK inhibitors 1 and 2, corresponding alkyne-containing analogs 3 and 4, and BTK-BRD4 PHICS 5 and 7 with corresponding inactive controls 6 and 8. 36C) Demonstration of covalent labeling of BTK in HEK293T cells by 3 and 4 at 1 μM via in-gel fluorescence. 36D) NanoBRET assay to evaluate ATP pocket occupancy and BTK inhibition. 36E) ATP pocket occupancy via NanoBRET for known BTK inhibitors 1 and 2 and their derivatives 3, 5, 4 and 7. 36F-36G) PHICS-induced phosphorylation of BRD4 by BTK in cells in the presence of 5 36F) or 7 36G) but not their inactive controls 6 36F) or 8 36G), respectively.

FIG. 37A-37E—37A) A click-chemistry-based platform for rapid assembly of PHICS. 37B-37C) Kinase inhibitors in which the leaving group is an aliphatic amine 37B) or aryl amine 37C). 37D) Target binder ligands for BRD4 (b), KRAS (c), and Myc (d). 37E) PHICS-mediated phosphorylation of BRD4 by FGFR4, ITK, and JAK3.

FIG. 38—Scheme 1. Cooperativity in three-body equilibria.

FIG. 39A-39C—39A) N-acyl-N-alkyl sulfonamides (NASA) follow a distinct structural motif with R1 and R2 serving as appropriate positions for modifications to achieve high atom economy and tunable reactivity towards hydrolysis or aminolysis. 39B) Synthesis of NASA derivative with various R1 and R2 groups allows reactivity tuning. 39C) —CH₂CF₃ is identified as a superior alternative because it retains significant aminolysis while it has attenuated hydrolysis.

FIG. 40A-40D—40A) Structures of NASA-alkynes designed and synthesized based on previously described high-affinity kinase inhibitors. The targeted kinase is provided in parenthesis. 40B) Convergent synthesis of the Lys targeting PHICS. The NASA-alkynes are combined with target-binder-azides via the highly chemo-selective CuAAC. 40C) In-vitro fluorescence labelling (with Cy5-dye) of purified kinases utilizing the corresponding NASA-alkyne probes. 40D) Ideal PHICS should have fine-tuned residence times. Molecules with high residence time act predominantly as inhibitors, while molecules with low residence times allow for partial activation of the kinase, facilitating phosphorylation of targeted protein.

FIG. 41—Challenges associated with the development of conventional bifunctional molecules. Similar PTM-inducing/removing chimeras are being developed by various labs and involve screening [e.g., using mass spectrometry (ABPP), DNA-encoded library or small molecule screening) to identify non-inhibitory binders of various PTM-inducing/removing enzymes. These approaches have several challenges: 1) Involves de novo ligand discovery and optimization, which is resource and time intensive 2) Identifying non-inhibitory pockets on enzymes is non-trivial and such pockets may be non-existent for certain enzymes 3) Discovered ligands will have unknown pharmacology (e.g., specificity, off-targets, PK/PD) 4) Mass spec methods primarily use small molecule fragments that have poor binding affinity. Applicants repurpose existing high-quality inhibitors of PTM-inducing/removing enzymes. Such inhibitors are abundantly available for nearly all PTM-inducing/removing enzymes.

FIG. 42—Example of kinase inhibitors that are available in plenty for PHICS development.

FIG. 43A-43C—PHICS can be developed using kinase inhibitors. A PHICS can be designed from a kinase inhibitor in which a nucleophile (e.g., cysteine) near the inhibitor-binding pocket can initiate an addition-elimination reactivity resulting in appending the target-protein binder to the kinase while releasing the inhibitor from the kinase's active site (43A). The kinase binder in such cysteine-triggered PHICS can be derived from abundantly available acrylamide-based inhibitors, where the amide nitrogen is often aryl-amine (43C) or less frequently aliphatic amine (43B). Using these BTK inhibitor scaffolds, Applicants synthesized BRD4 PHICS with aryl amine scaffold (43C) or aliphatic amine scaffold (43B), wherein the inhibitor is connected to BRD4 binder JQ1 via cleavable methacrylamide linker. Applicants also synthesized Iphics (inactive controls using (R)-JQ1 that does not bind to BRD4) and tested in PHICS can rewire BTK's specificity and induce phosphorylation of BRD4. Indeed, Applicants observed much higher BRD4 phosphorylation in the presence PHICS than with iPHICS. Applicants also observed significantly higher levels of co-immunoprecipitated BTK-FLAG and higher levels of BRD4 phosphorylation in presence of PHICS than that of iPHICS. Beyond BTK, Applicants have successfully developed recruiting ligands for EGFR, FGFR (pan), FGFR4, ITK, JAK3, and CDK2.

FIG. 44A-44C—Data showing group transfer reactivity in cells. Applicants performed target engagement studies using compounds that have alkyne handle in place of JQ1 (41A) and confirmed that the scaffolds covalently engaged BTK in cells (41B). Briefly, HEK293T cells transiently expressing BTK were treated with alkyne-containing compounds for 4 h. After washing with PBS and lysis, cell lysates were introduced to Cu-catalyzed click reaction with sulfo-Cy5.5 azide and labelling of BTK was confirmed via in-gel fluorescence (41B).

FIG. 45A-45E-Data showing elimination of inhibitor from ATP pocket. To demonstrate that the BTK inhibitor scaffold is released from the ATP-binding pocket, we used a reported assay based on Bioluminescence Resonance Energy Transfer (BRET) between a nanoluciferase (nanoLuc) and a fluorophore probe that binds to the ATP pocket; a higher occupancy of this ATP pocket by the inhibitor will prevent the binding of the tracer and lower the BRET signal (42A). As expected, acrylamide-based BTK inhibitors (42B, 42C) dramatically lower BRET signal indicating blocking of ATP-pocket, while their derivatives with cleavable methacrylamide linkers (alkynes or PHICS, 42D and 42E) did not affect binding of tracer.—In congruence with these findings, we observed higher autophosphorylation of BTK with PHICS compounds compared to the parent acrylamide inhibitors. Applicants have extended this approach to EGFR, FGFR, ITK, JAK3, and CDK2 kinases.

FIG. 46—Challenges surrounding development of Cys reactive linkers. Binding moieties BTK, EGFR, FGFR (pan), FGFR4, ITK, JAK3, CDK2, BMX, AKT, JNK1.

FIG. 47—Unmet need: Phosphorylation of any protein on demand using small molecules.

FIG. 48—Phosphorylation converts neutral residue to a—vely charged residue.

FIG. 49—Kinase #1. AMP-activated protein kinase (AMPK).

FIG. 50—Kinase #2. Protein kinase C required specificity and MoA rewiring.

FIG. 51A-51B—Negishi coupling route to benzolactam: 9 steps, 22% yield.

FIG. 52A-52B—Gain-of-function: PHICS can rewire kinase specificity.

FIG. 53—PHICS are “catalytic” and show turnover.

FIG. 54—PHICS are “catalytic” and show turnover.

FIG. 55—The Hook effect: The 3-body vs. 2-body equilibrium.

FIG. 56—PHICS displays higher specificity than sum of the two parts.

FIG. 57A-57E—Kinase #3. Abelson kinase for tyrosine phosphorylation.

FIG. 58A-58C—Evidence for addition and elimination reactivity in cells.

FIG. 59—Evidence for addition and elimination reactivity in cells.

FIG. 60—Challenges surrounding development of Cys reactive linkers.

FIG. 61—Replacement of nitrile with fluoro groups reduce hydrolysis rate by 5-fold.

FIG. 62—A platform for pharmacologic protein editing using inhibitors of writers and erasers. (de) indicates both directions of the modification, e.g., (de)phosphorylation may mean phosphorylation and/or dephosphorylation.

FIGS. 63—C1 domain: A miniature chemogenetic tag of human origin.

FIGS. 64—C1 domain: A miniature chemogenetic tag of human origin

FIG. 65—PHICS can induce naturally-occurring phosphorylation and signal transduction.

FIG. 66—PHICS can induce naturally-occurring phosphorylation and signal transduction.

FIG. 67—PHICS-triggered phosphoryulation can induce Liprin phase separation.

FIG. 68—PHICS can recruit atypical kinase BRD4 to induce Myc degradation.

FIG. 69A-69C—PHICS induced BCR-ABL phosphorylation and sequestration.

FIG. 70—Homo-PHICS increases pY253 located in the ATP-binding loop.

FIG. 71—Homo-PHICS: Neo-phosphorylation mediated inhibition of a onco-fusion kinase. Homo-PHICS: Kills onco-fusion cancer lines with ˜6 nM; Kills cancer lines with resistant mutants better than known drug; Are more effective than known drugs in other onco-fusion cell lines; MOA is neo-phosphorylation and can be potentially extended other fusions.

FIG. 72A-72B—Homo-PHICS is an allosteric binder, but not an inhibitor of ABL.

FIG. 73—Homo-PHICS dimerizes ABL.

FIG. 74—Homo-PHICS blocks downstream signaling of BCR-ABL.

FIG. 75—Homo-PHICS induce apoptotic cell death.

FIG. 76—PRISM profiles compounds in 1,000 cancer cell lines of diverse lineages/dependencies. Homo-PHICS PRISM data shows selectivity.

FIG. 77—Homo-PHICS is functional in the presence of active site mutants.

FIG. 78—Drug resistance profiles of asciminib and VS1150. Treatment dose was increased over time (d0-7:1 nM ABL001, 20 nM VS1150; d7-14:2 nM ABL001, 40 nM VS1150; d14-21:4 nM ABL001, 80 nM VS1150; d21-63:100 nM ABL001, 500 nM VS1150).

FIG. 79—Statistically significant gRNA enrichment for Asciminib. Red underline in enrichment plot corresponds to gRNA that are being followed up on in subsequent single guide experiments.

FIG. 80—Statistically significant gRNA enrichment for Asciminib but not for VS1150. Red underline in enrichment plot corresponds to gRNA that are being followed up on in subsequent single guide experiments.

FIG. 81—PHICS can induce inhibitory neo-phosphorylations on known substrates.

FIG. 82A-82C—PHICS allows control of hyperactive variant of BTK.

FIG. 83—PHICS allows control of hyperactive variant of BTK: Targeted polypharmacology.

FIG. 84—ABL/PKC/AMPK PHICS induces death of ibrutinib-resistant cells.

FIG. 85—PHICS can hyperphosphorylate and inhibit K-Ras signaling.

FIG. 86—PHICS can hyperphosphorylate and inhibit K-Ras signaling.

FIG. 87—Generalization to other enzymes for PTM addition or removal.

FIG. 88—˜10 analogs synthesized to attain nM potencies.

FIG. 89—PHICS Kinases.

FIG. 90—AKT phosphorylation on BRD4.

FIG. 91A-91C—(91A) Current design of dumbbell-like bifunctional molecules. (91B) Ternary complex formation with dumbbell-like bifunctional requires allosteric non-inhibitory binders (green triangle). The PTM is facilitated because the natural substrate (blue triangle) can bind to the active site. (91C) Our new design can expand to active site inhibitors. Michael addition followed by a retro-aza-Michael allows for the natural substrate to bind and promote PTM on the POI.

FIG. 92A-92C—(92A) Methacrylamides 4-6 with alkyne handle from acrylamide 1-3 for Cys labeling. (92B) Reaction scheme for reactivity assessment of methacrylamides 4-6 with N—Ac-Cys-OMe in PBS buffer at rt. (92C) Kinetics data of methacrylamides with rate constant.

FIG. 93A-93H—(93A) Reaction scheme for designing CGT-PHICS for FGFRs targeting BRD4 (93B, 93C, 93D) Nano-BRET assay for binding pocket occupancy for known FGFR inhibitors Fisogatinib, Futibatinib, PRN1317 and their non-covalent derivatives and CGT-PHICS. (93E, 93F, 93G, 93H) CGT-PHICS induced phosphorylation of BRD4 by FGFR4 and FGFR2 in HEK293T cells.

FIG. 94A-94C—(94A) Schematic for the dPHICS induced dimerization of FGFR receptors and signaling. (94B) dPHICS design for the FGFR dimerization. (94C) MAPK pathway activation in the presence of dPHICS.

FIG. 95A-95J—Development of PHICS using Cysteine group transfer chemistry. (95A) Structures of the respective kinase (JAK3, ITK and EGFR) Non-covalent binders (12A-16A), Covalent inhibitors (12-16), methacrylamides (17-21), and PHICS (22-26). (95B) Kinetic data of methacrylamides (17-21) with rate constant. (95C, 95D, and 95E) Nano-BRET assay comparing occupancy of Covalent (12, 13 and 14), non-covalent (12A, 13A and 14A) and CGT-PHICS (22, 23 and 24) of JAK3 (95C) and ITK (95D and 95E). (95F, 95G, 95H, 95I and 95J) PHICS-induced phosphorylation of BRD4 by the respective kinases, JAK3, ITK and EGFR expressed in HEK293T cells.

FIG. 96A-96E—(96A) A BTK-BRD4 PWC using BTK inhibitor, 2.1. (96B) BRET assay confirms that while 2.1 inhibits BTK, PWC 2.2 does not. (96C) BRD4 phosphorylation by PWC 2.2, but not by 2.3 (inactive control). (96D) An array of twenty lysine group-transfer linkers with tunable reactivity. (96E) Validation of PWCs that use Cys/Lys group-transfer chemistry using various kinase inhibitors. Excess BRD4 binder (JQ1) was used as competitor (comp). Red: BRD4 phosphorylation. Green: Total BRD4.

FIG. 97—Examples of Ar leaving groups. R1, R2, R3 can be alkyl, Ar, or heteroatoms such us O, N etc.

FIG. 98—Examples of Bifunctionals with sulfonamide based leaving groups.

FIG. 99—Kinetics of thiol addition for FDA approved drugs Osemartinib, Decomatinib, and pyrotinib after derivatization of their aniline group to a sulfonamide. JK4 consists of an example of R1 to trifluoroethyl group. JF70 and JF74 represent Ar-thio leaving groups at two different oxidative states.

FIG. 100A-100B—NASA warheads for targeting nucleophilic residues in proteins. (100A) Conversion of the Ar-sulfonamide to alkyl sulfonamide does not impact Lysine reactive (same % yield) nor aqueous stability (comparable hydrolysis rates). (100B) Replacement of Cyano group with pi acceptors or water attracting groups leads to decreased stability. The opposite effect is observed upon replacement with hydrophobic fluoro groups such as CF3 Different orientation of the sulfonamide bond was observed after X-ray analysis. The cyano group orients proximal to the carbonyl facilitating n->pi* interaction which increases the electrophilicity of the carbonyl. The CF3 is placed opposite site of the carbonyl.

FIG. 101—Examples of leaving groups with additional/other substitutions.

FIG. 102—Experimental data validating the platform described herein. Example linkers described herein successfully generated PHICS molecules. The results show novel cysteine group transfer based PHICS induced BRD4 phosphorylation using BTK.

FIG. 103—The large molecular weight of heterobifunctional molecules manifested in their poor PK profiles and my impact binding affinity of the ligand to the protein. With this goal in mind, we have reduced the size of the warheads without perturbing reactivity towards lysine.

FIG. 104A-104C—(104A) Aminolysis and Hydrolysis scheme of NASA. Hydrolysis generates the carboxylate and sulfonamide components of NASA, which in the context of Chimeras can act as competitors. (104B) —CF3 based NASA show enhanced hydrolytic stability. (104C) X-ray analysis. N31 possibly destabilizes carbonyl via n->p* donation while the N32 is placed in the opposite direction, protecting the carbonyl through its hydrophobicity.

FIG. 105—Library of tunable NASA derivatives.

FIG. 106—Heterocycles are privileged scaffolds in medicinal chemistry and offer the opportunity fragment-based ligand discovery and further tuning by controlling the electronic properties of the ring.

FIG. 107—Schematic of Nano-BRET assay.

FIG. 108—Tuning reactivity for targeting aryl amines.

FIG. 109—Evidence of cysteine group transfer in cells. Covalent labeling of FGFR4 and BTK in cells in gel fluorescence.

FIG. 110—Tunable group transfer warheads targeting cysteine and lysine.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Polypeptide labeling has been classically developed to obtain spatially distributed molecular localization information. Electrophilic reactive linkers disclosed herein define new classes of polypeptide labeling capable of expanding the function of polypeptides and the molecules labeling them. In general, electrophilic reactive linkers comprise of an electrophilic reactive group and one or more linkers. Electrophilic reactive linkers can be used to create neo-post translational modifications (neo-PTMs) and/or neo-protein-protein interactions (neo-PPIs).

In one aspect, electrophilic reactive groups can be configured to facilitate the covalent labeling of a polypeptide (e.g., protein) with a target-binding moiety. The labeling of a polypeptide with a target binding moiety can be used to define new substrates not normally targeted or modified by such proteins. In such embodiments, the electrophilic reactive linkers comprise a modifying polypeptide binding moiety linked to a target binding moiety. The modifying polypeptide binding moiety non-covalently binds to the modifying polypeptide of interest and, as a result, leads the electrophilic reactive linker—via proximity-driven reactions—to “label” the protein by covalently binding the target binding moiety to a nucleophile located on the polypeptide. In such embodiments, the modifying polypeptide binding moiety is then released from the labeled modifying polypeptide, either by inherent kinetics of the molecule or by application of a quencher. The target binding moiety may then direct the labeled modifying polypeptide to bind and modify new target substrates. This approach also expands the number of modifying polypeptide binders that may be used. For example, there are several high-quality kinase inhibitors that exist, but such inhibitors are unsuitable for use in targeting chimeras that remain bound to the kinase and thus may otherwise inhibit enzymatic activity of the bound kinase. As discussed in further detail below, selection of appropriate inhibitors, and the optional use of quenching molecules, allows these inhibitors to be used in the aforementioned labeling process without impacting the downstream modification reaction.

In one aspect, embodiments disclosed herein provide a bifunctional molecule comprising a electrophilic reactive linker and a target polypeptide binding moiety attached to the linker on one end and a modifying polypeptide binding moiety attached to the linker on the opposite end. For example, the modifying polypeptide binding moiety binds to a phosphatase, a ubiquitinase, a deubiquitinase, an acetyltransferase, deactylase, methyltransferase, demethylase, or glycosyltransferase. The target polypeptide binding moiety binds to a polypeptide to which the modifying moiety is to be attached and modified by the modifying moiety. For example, the target polypeptide may be a rogue endogenous polypeptide or a polypeptide located on the surface of a pathogenic bacteria.

### Electrophilically Reactive Linkers

In another aspect, embodiments disclosed herein are directed to an electrophilic linker having enhanced stability, reactivity, and tuneability. In one example embodiment the linker molecule has the formula L₁-El, EL-L₁, or L₁-El-L₂, wherein L₁ and L₂ are independently selected from alkane, alkene, amine, either, thiol, sulfone, carbonyl, acyl, ketone, carboxylate ester, amide, enone, anhydride, imide, and PEG, and wherein EL is an electrophilic reactive group. The electrophilically reactive linkers may be used to attach all or a portion of L₁ or L₂ to a target polypeptide. The El group of the linker molecule may be selected based on an ability to covalent attach all or a portion of L₁ or L₂ to a cysteine, a lysine, a methionine, or a tyrosine residue on the target polypeptide. In one example embodiment L₁ and L₂ may independently be any of the linkers described above.

### Cysteine Reactive Electrophilic Linkers

In one example embodiment, the linker molecule comprises and El group suitable for f attachment of all or a portion of L₁ or L₂ to a cysteine, and wherein the El is selected from the group consisting of:

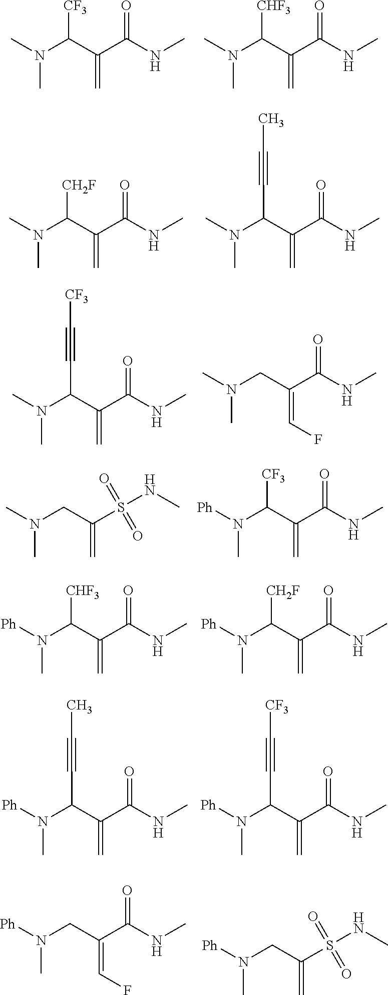

### Lysine Reactive Electrophilic Linkers

In one example embodiment, the linker molecule comprises and El group suitable for f attachment of all or a portion of L₁ or L₂ to a lysine, and wherein El is

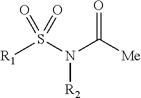

wherein R₁ is selected from the group consisting of;

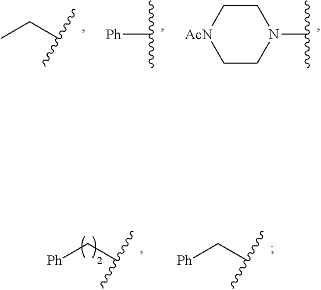

and
wherein R₂ is selected from the group consisting of

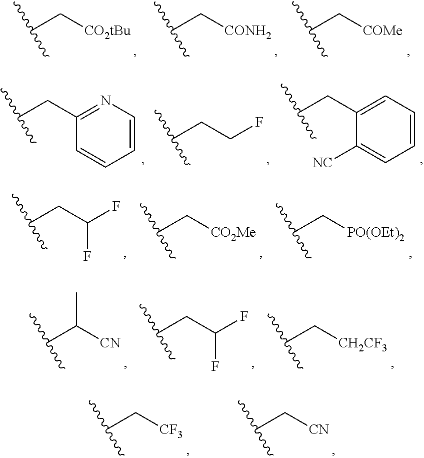

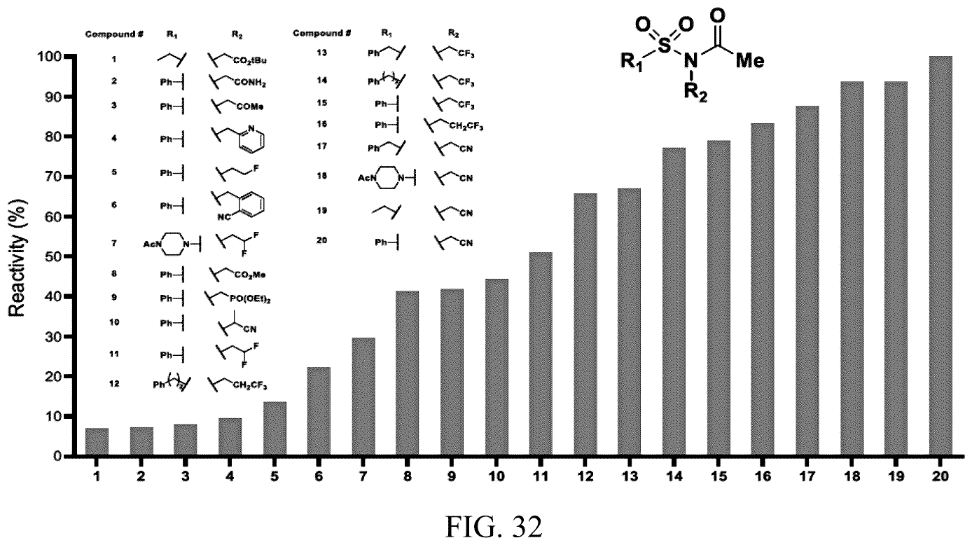

FIG. 32 demonstrates how the reactivity and specificity of the El group can be tuned to desired levels by modifying substituents on the El group.

In another example embodiment, the linker molecule comprises an El group suitable for attachment of all or a portion all or a portion of L₁ or L₂ to a lysine, wherein the El group is selected from;

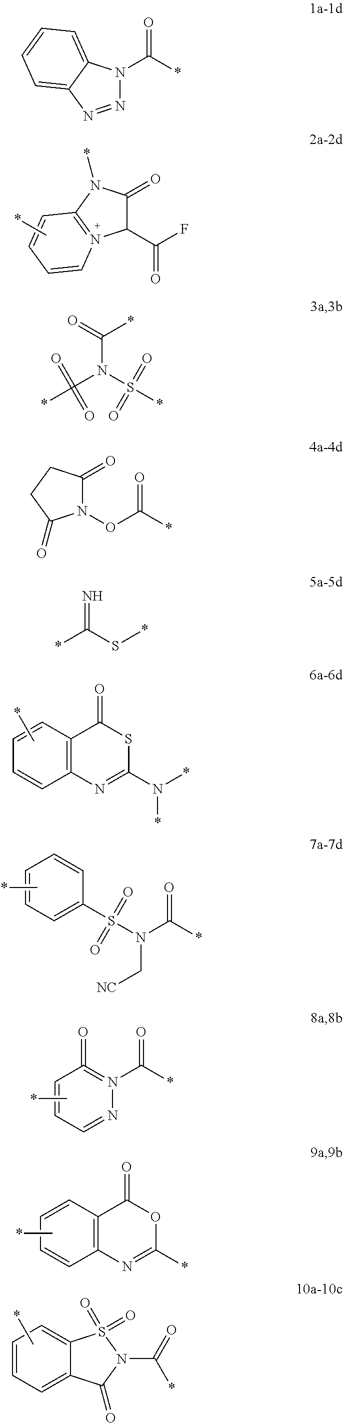

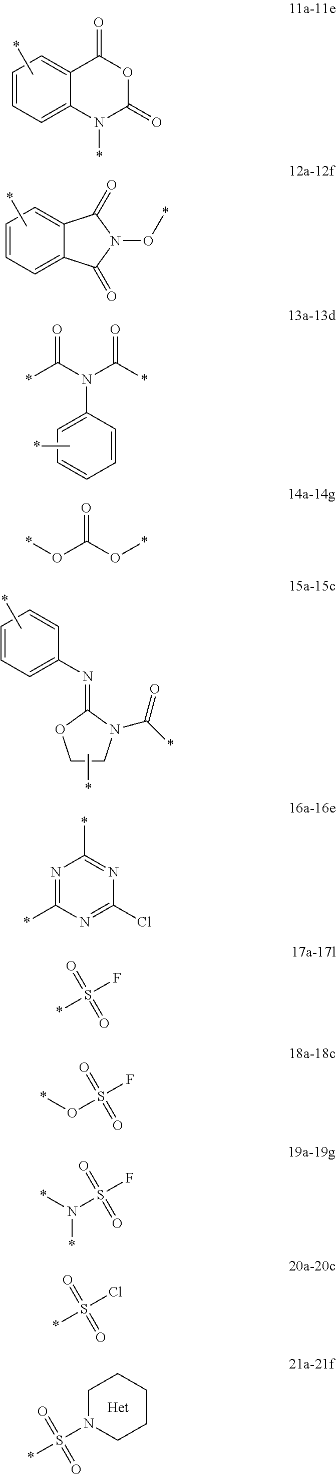

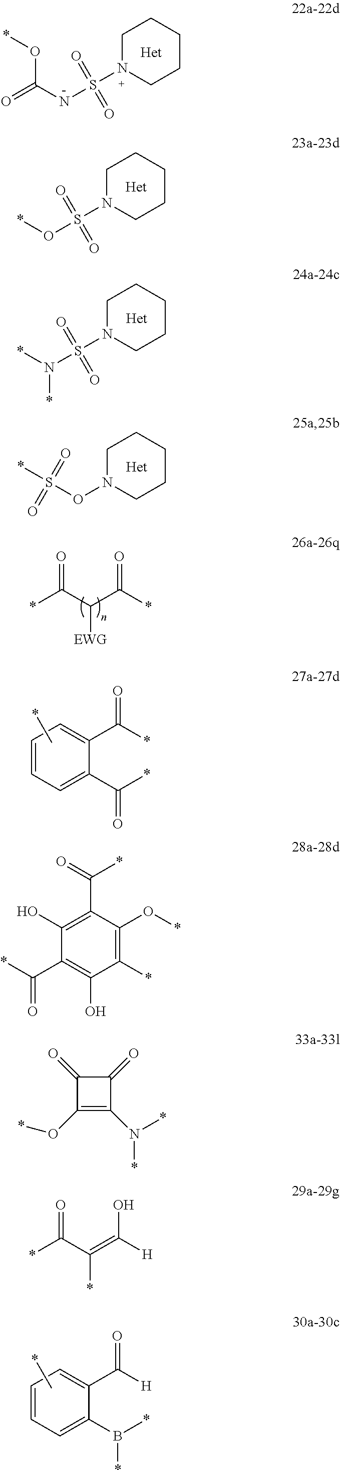

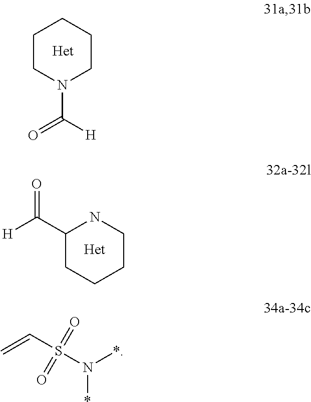

### Methionine Reactive Electrophilic Linkers

In another example embodiment, the linker molecules comprises an El group suitable for attachment of all or a portion all or a portion of L₁ or L₂ to a methionine, wherein the El group is selected from;

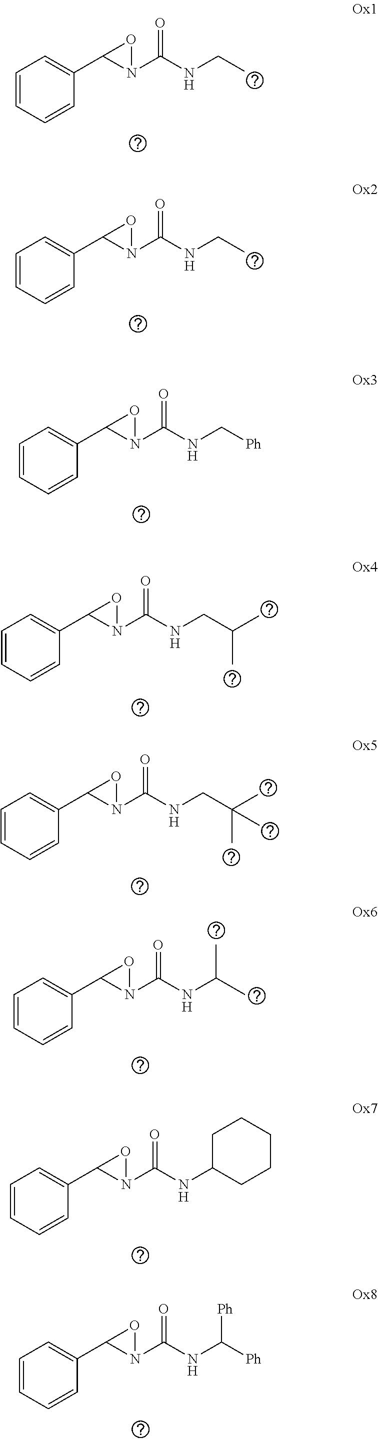

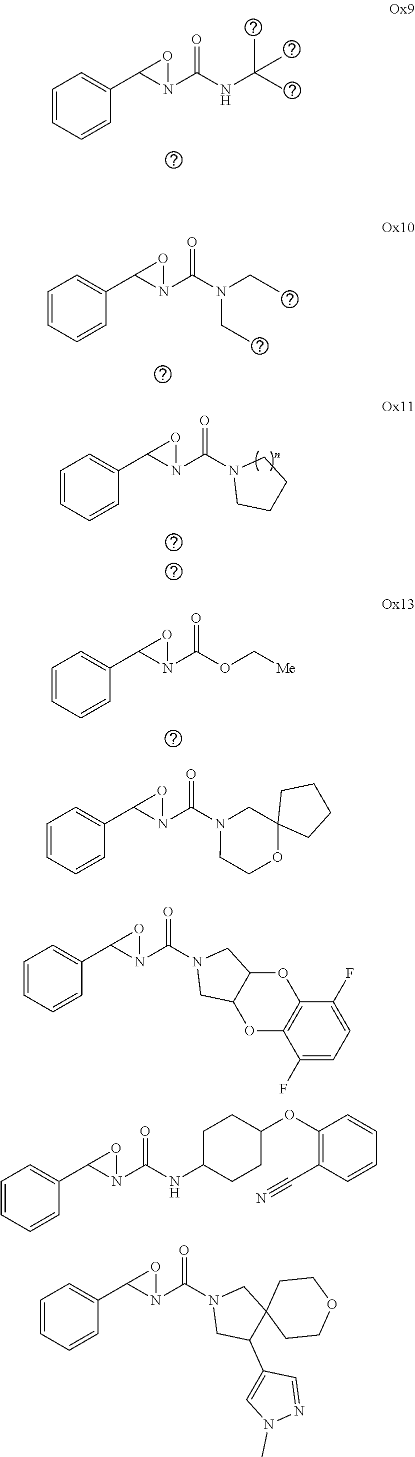

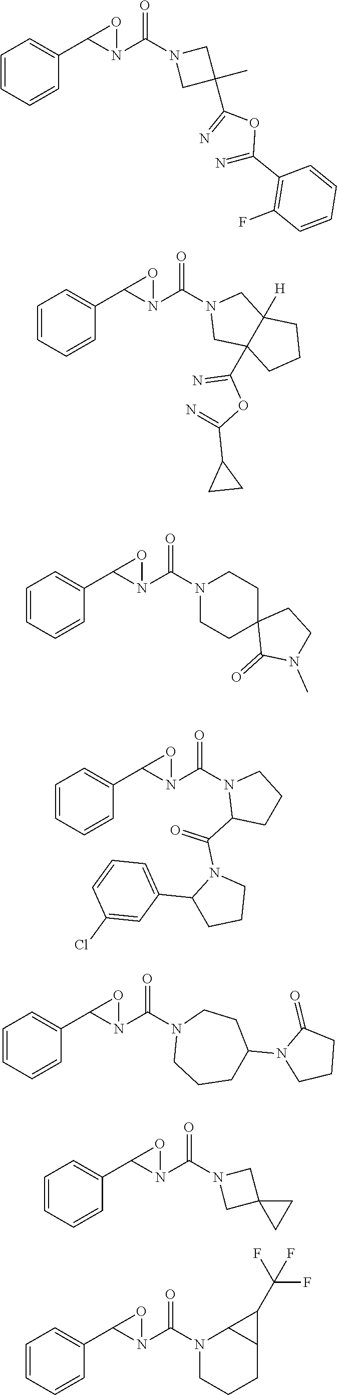

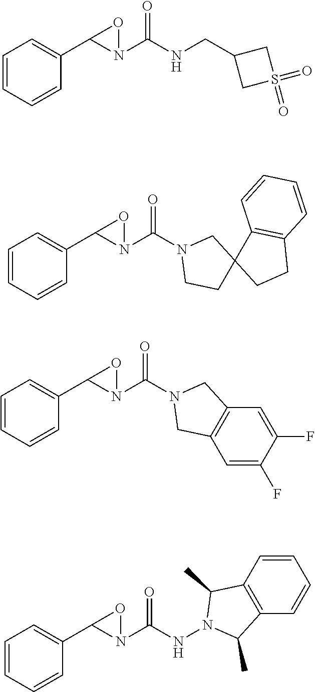 [SMILES: O=C(NCC1CS(=O)(=O)C1)N1OC1c1ccccc1 ; O=C(N1CCC2(CCc3ccccc32)C1)N1OC1c1ccccc1 ; O=C(N1Cc2cc(F)c(F)cc2C1)N1OC1c1ccccc1 ; C[C@@H]1c2ccccc2[C@H](C)N1NC(=O)N1OC1c1ccccc1]

### Use of Electrophilically Reactive Linker

The electrophically reactive linkers disclosed in this section may be used in any bifunctional molecule, including the chimeric molecules disclosed in this application. The electrophilically reactive linkers may also be used in other bifunctional molecules such as PROTACS. Békés et al. “PROTAC targeted protein degraders; the past is prologue” Nature Review Drug Discovery 21, 181-200 (2022). In addition, the electrophically reactive groups may also be used with other bifunctional molecules. For example, a target polypeptide binding moiety may be attached to one of the linker and a modifying moiety may be attached to the other end of the linker. The target polypeptide binding moiety may be selected to selectively bind a target polypeptide, such as an enzyme, at a catalytic or allosteric site to allow for the electrophilically reactive linkers disclosed herein to covalently attach the modifying moiety to the target polypeptide at cysteine, lysine, methionine, or tyrosine. In one example embodiment, the target polypeptide binding moiety is a binding moiety capable of specifically binding a phosphatase, a ubiquitinase, a deubiquitinase, an acetyltransferase, deactylase, methyltransferase, demethylase, or glycosyltransferase and the modifying moiety binds a neo-substrate for the kinase, a phosphatase, a ubiquitinase, a deubiquitinase, an acetyltransferase, deactylase, methyltransferase, demethylase, or glycosyltransferase. In another example embodiment, the modifying moiety is an immunogenic moiety and the target polypeptide binding moiety is a polypeptide to which the immunogenic moiety is to be attached.

### Electrophilic Reactive Group

In an example embodiment, the electrophilic reactive linkers as disclosed herein may comprise an electrophilic reactive group. In an example embodiment, the electrophilic reactive group is located between a linker and target binding moiety. In an example embodiment, the electrophilic reactive group is located between a linker and modifying polypeptide binding moiety (i.e., modifying binding moiety). In an embodiment, the electrophilic reactive group is located between two linkers, a first linker attached to the modifying polypeptide binding moiety and a second linker attached to the target binding moiety. An electrophilic reactive group, as used herein, is typically a functional group that can form a reversible or irreversible bond with a nucleophilic functional group. The electrophilic reactive group allows for the target binding moiety to directly attach to a target polypeptide (e.g., a kinase). Upon attaching to the electrophilic reactive group, the target polypeptide is now tagged with the target binding moiety. In an example embodiment, the molecules or binding moieties may be modified at an electrophilic reactive group to reduce or lessen the strength of covalent binding capabilities of an electrophilic reactive group, or to increase the binding affinity or strength of binding of an electrophilic reactive group as desired according to the application. In an embodiment, a binding molecule may be chosen that would create irreversible covalent binding at a target. When used in a chimeric small molecule (e.g., bifunctional molecule), such tight bonding may be less desirable. Thus, modification of such electrophilic reactive group would be desirable and can be modified to reduce the interaction, see, e.g., sciencedirect.com/science/article/pii/S0968089618320807. In particular, reactivity can be designed to allow for covalent binding at the target, with reversible or irreversible properties, depending on desired functionality. In an embodiment, the electrophilic reactive group is designed to react with an amino acid side chain reactive group. The amino acid side chain reactive group may be nucleophilic. The nucleophilic amino acid side chain reactive group may comprise Cysteine, Serine, Threonine, Tyrosine, Glutamic Acid, Aspartic Acid, Lysine, Arginine, and Histidine, or a Methionine. In one preferred embodiment, the electrophilic reactive group reacts with lysine. An exemplary database that can aid in identification for protein ligand interaction around the binding site is described in Du et al., Nucleic Acids Research, Volume 49, Issue D1, 8 Jan. 2021, Pages D1122-D1129, incorporated herein by reference, with the database, CovalentInDB accessible at cadd.zju.edu.cn/cidb/. The approach can be used with any design of the electrophilic reactive group for molecules as disclosed herein.

### N-Acyl-N-Alkyl Sulfonimide (NASA)

NASA chemistry may be used to accomplish the design of the electrophilic reactive group by forming a reversible or irreversible bond with a nucleophilic functional group located on the modifying polypeptide. NASA chemistry is generally described in Nat Commun 9, 1870 (2018), incorporated herein by reference. In preferred embodiments, the modifying polypeptide binding moiety can be attached to a linker utilizing N-acyl N-alkyl sulfonamide (NASA) electrophilic reactive group further attached to a modifying polypeptide binding moiety. The chimeric small molecule containing a NASA will, upon non-covalent binding to a target enzyme, covalently bond to the enzyme as the NASA chemically reacts with a proximal lysine or other amino acid as described herein. The NASA modified chimeric small molecule then disassociates from the modifying polypeptide leaving behind the protein targeting binder covalently attached to the modifying polypeptide. This modified modifying polypeptide will then bind to the target protein through the newly attached binder and further modify the protein. In an example embodiment, NASA chemistry is used to label a modifying polypeptide binding moiety. Accordingly, an embodiment comprises methods of making compositions disclosed herein using NASA chemistry, and as further described in the examples.

In an example embodiment, a NASA analogue has the formula:

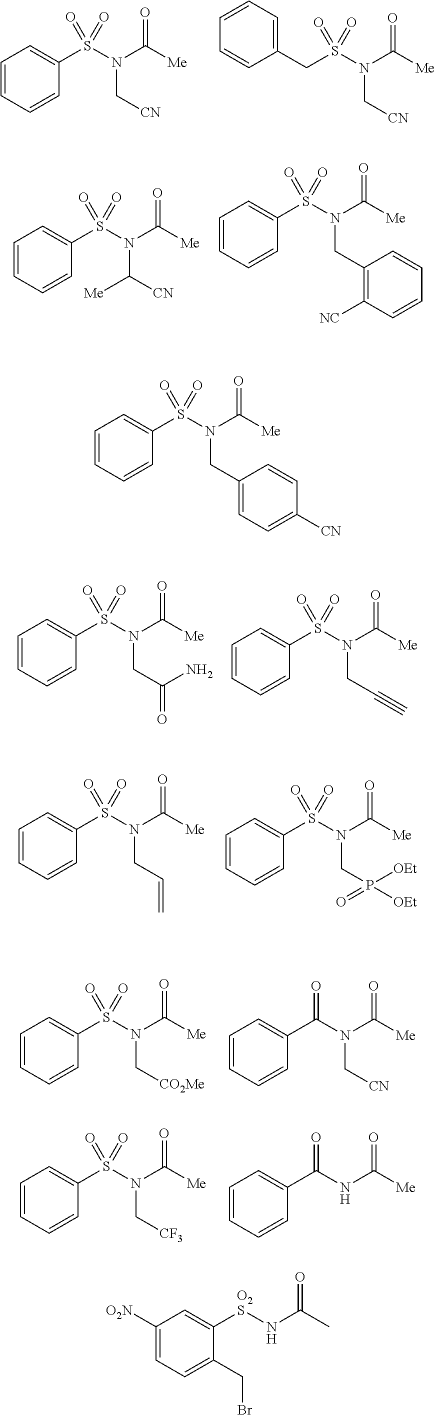

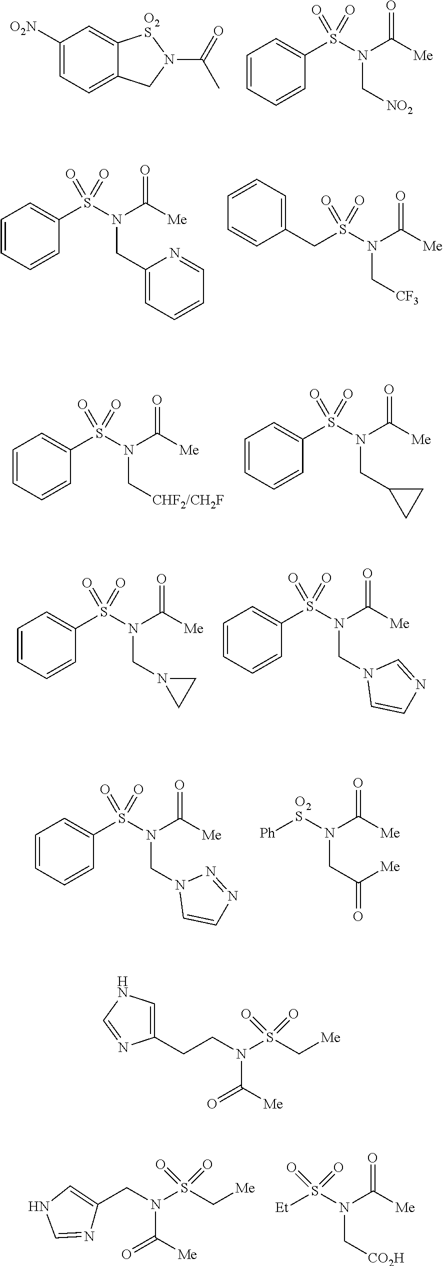

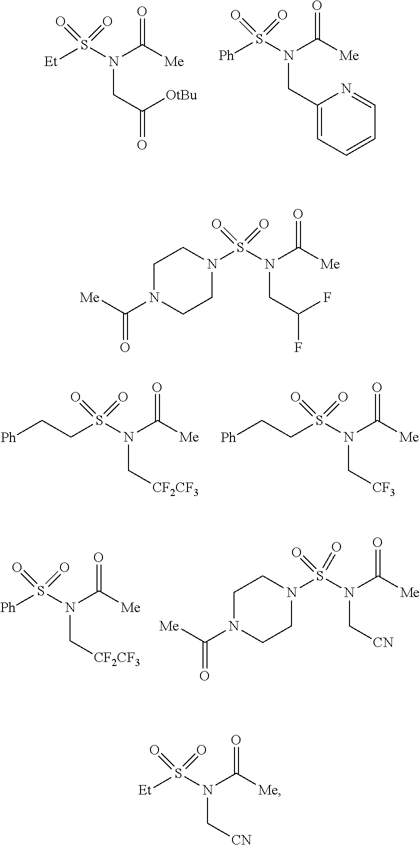

or a derivative thereof.

### Dibromophenyl Benzoate

In preferred embodiments, the electrophilic reactive group is dibromophenyl benzoate (DB). DB can be used to functionalize a linker by reacting with a nucleophile located on a modifying polypeptide. The dibromophenyl group acts as the leaving group facilitating the reaction while the benzoate stabilizes the now attached moiety. In a preferred embodiment, a linker connecting a modifying polypeptide binding moiety and target binding moiety is functionalized with DB to label a modifying polypeptide with the target binding moiety. DB chemistry is generally described in Takaoka et al. Chem. Sci., (2015), 6, 3217-3224, incorporated herein by reference.

### N-Sulfonyl Pyridone

In preferred embodiments, the electrophilic reactive group is N-sulfonyl pyridone (SP). SP can be used to functionalize a linker by undergoing sulfonylation with a nucleophile located on a modifying polypeptide. In a preferred embodiment, a linker connecting a modifying polypeptide binding moiety and target binding moiety is functionalized with SP to label a target modifying polypeptide with the target binding moiety. SP chemistry is generally described in K. Matsuo et al. Angew. Chem. Int. Ed. 2018, 57, 659 incorporated herein by reference.

In one example embodiment, the electrophilic reactive group comprises one of

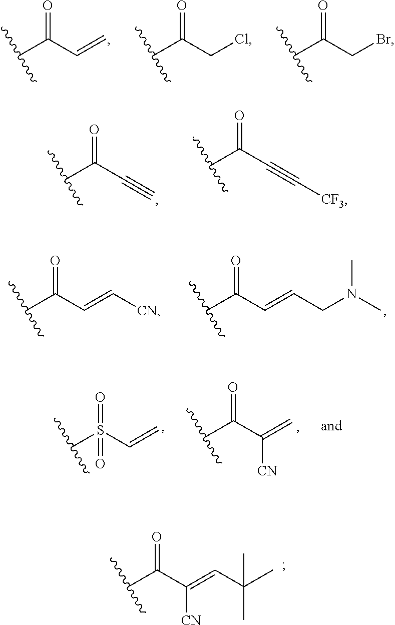

In example embodiments, the electrophilic reactive group is of the formula:

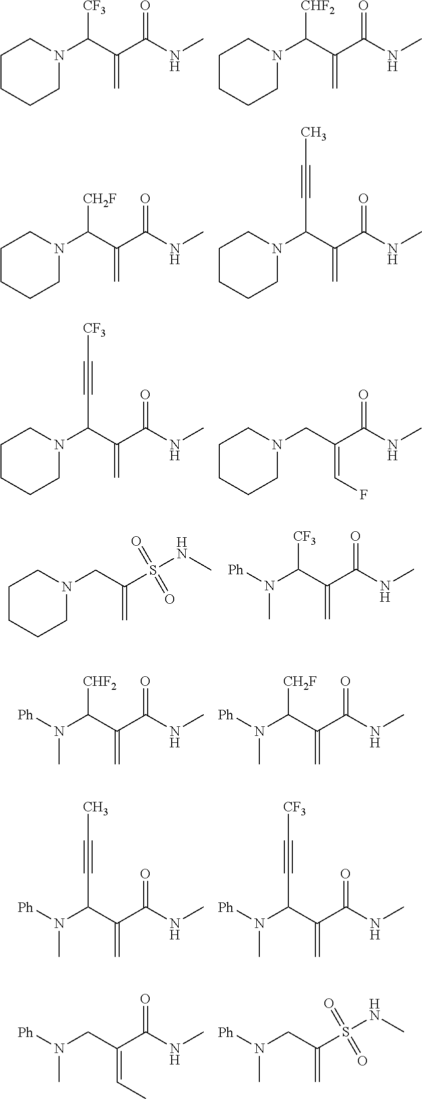

or a derivative thereof.

### Photo-Reactive Group

In one example embodiment, the electrophilic reactive group is a photo-reactive group. In one embodiment, the photo-reactive group is a photoactivated cell-surface reactive group. In another embodiment, the photoactivated cell-surface reactive group is a benzophenone, azide, or diazirine, wherein the group is activated to become a carbon-centered radical, nitrene, or carbene, respectively. In another embodiment, the photo-reactive group is a thienyl-substituted alpha-ketoamide, see e.g. Ota, E., et al. “Thienyl-Substituted α-Ketoamide: A Less Hydrophobic Reactive Group for Photo-Affinity Labeling.” ACS Chem. Biol. 2018, 13 (4), 876-880.

### Additional Electrophilic Reactive Linkers

In example embodiments, the electrophilic reactive linkers comprise:

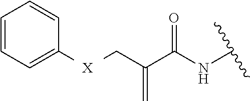

wherein X is selected from the group consisting of:

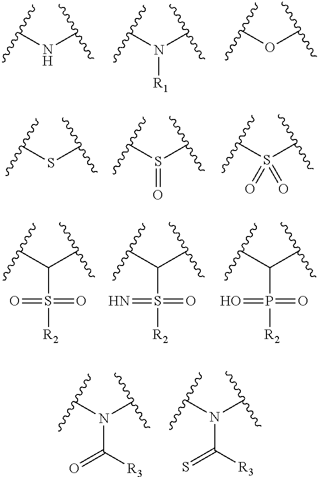

wherein R1, R2, R3 individually comprise of an alkyl group, aryl group, or a heteroatom optionally O, N, or S.

In example embodiments, the electrophilic reactive group is

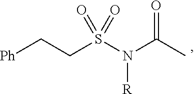

wherein R is selected from the group consisting of:

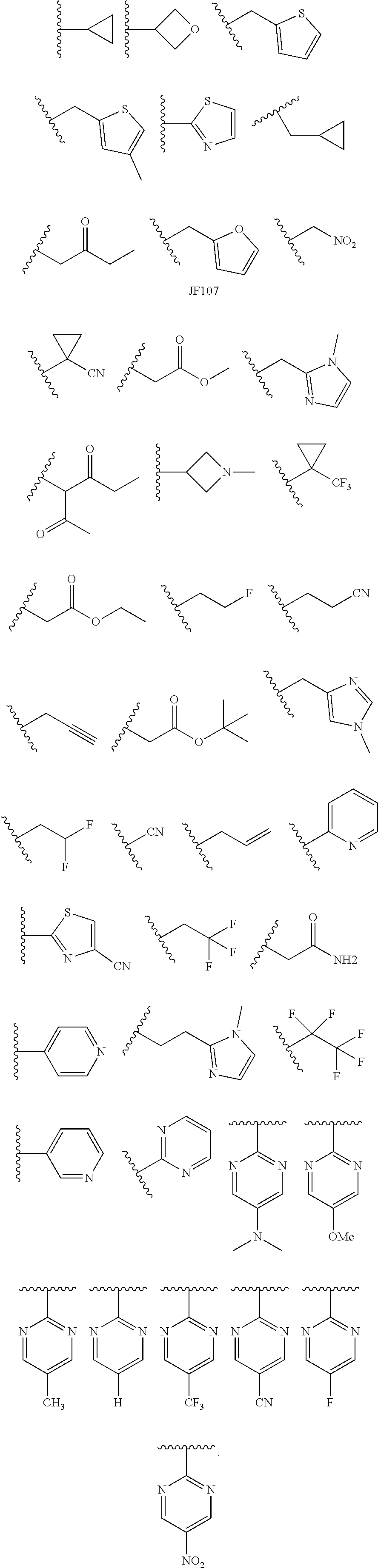

In example embodiments, the electrophilic reactive group is

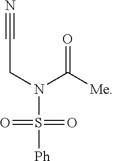 [SMILES: CC(=O)N(CC#N)S(=O)(=O)c1ccccc1]

In example embodiments, the electrophilic reactive group is

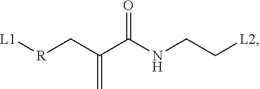

wherein R is selected from the group consisting of:

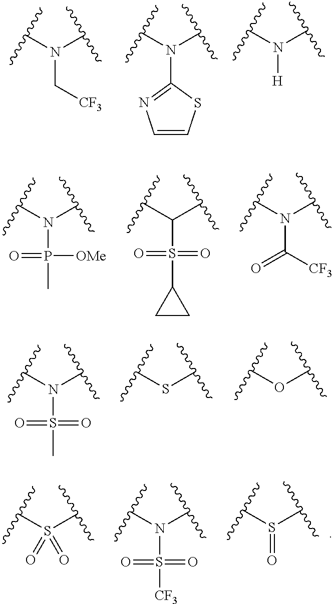

### Linker

A linker or linking moiety is a bifunctional or multifunctional moiety that can be used to link one or more of target binding moiety, protein binding moiety. In some embodiments, the linker has a functionality capable of reacting with the moieties for covalent attachment. The linker moiety is preferably a chemical linker moiety and is represented in the formulas of the present invention as L. In an embodiment, the linker moiety may preferably comprise one or more repeats, e.g. 1, 2, 3, 4, 5, 6, 7, 8 or more repeats, which may be utilized to facilitate or improve spacing, conformation, and/or performance of the molecules. The linker described herein may refer to both L1 and L2 or L1 and L2 are different linkers described herein.

There are many linkers that can be used to design the compounds described herein. For example, amides, esters, and amines can be created by well-known reactions and are used in medicinal chemistry. Additional linkers such as oxygen-containing linkers (e.g., ethers, ketones, esters, etc.,) are used create natural product-like molecules. Linkers can be designed to connect two or more binding moieties, for example, a polypeptide binding moiety and a target binding moiety. These linkers can be advantageous for extended binding sites, such as those described herein. Moreover, the linkers are designed to include an electrophilic reactive group. Linkers can be taken from, for example, ChEMBL and/or ZINC databases and designed to connect two or more binding moieties and include an electrophilic reactive group. When selecting a linker, it is common to compute and compare their properties such as their topographical characteristics and/or electronic properties. For further guidance on linker selection see e.g., P. Ertl, et al., Bioorganic & amp; Medicinal Chemistry 2023, 81, 117194, incorporated herein by reference.

In an embodiment, a linker can include an alkane, alkene, amine, either, thiol, sulfone, carbonyl, acyl, ketone, carboxylate ester, amide, enone, anhydride, imide, PEG, or any combination thereof.

A linker or linking moiety can be used to link a modifying polypeptide binding moiety to the target binding moiety, and/or the electrophilic reactive group to either the modifying polypeptide binder, target binding moiety, or both. When more than one linker molecule is used in a molecule, the linkers may be the same or different from each other.

In one example embodiment, the linker may be represented with an exit vector. In one example embodiment, the exit vector may be represented independently of the linker. Exit vector parameters can be identified in part based on average orientation of a substituent attached to a variation point which can be generated using chemoinformatics software. An exit vector may comprise outgoing bonds from a chemical moiety. In an embodiment, the exit vector is provided as bonds on the linker or from the binding moiety, providing conformation of attachment between the linker and the activator moiety and/or the localizing moiety.

### Exit Vectors

One or more exit vectors may be utilized with the molecules described herein. In certain embodiments, the linker or modifying polypeptide binding moiety may be represented with an exit vector comprised in the linker or modifying polypeptide binding moiety. In an embodiment, the exit vector may be represented independently of the linker or modifying polypeptide binding moiety. Exit vector parameters can be identified in part based on average orientation of a substituent attached to a variation point which can be generated using chemoinformatics software. An exit vector may comprise outgoing bonds from a chemical moiety. In an embodiment, the exit vector is provided as bonds on the linker or from an Abl binding moiety, providing conformation of attachment between the linker and the Abl binding moiety and/or the second Abl binding moiety. The exit vector may also be represented independent of the linker of the formulas detailed herein. In an embodiment, the exit vector is comprised in W.

In an embodiment, the bond is chosen to be energetically favorable, preferably increasing binding affinity. The exit vector may be adjusted depending on the linker utilized in the molecules. In embodiments, the exit vector is a chemical moiety or bond that facilitates stereochemical protrusion that may further facilitate subsequent coupling, bonding and/or accessibility.

In one example embodiment, the modifying polypeptide binding moiety has an adapter or reactive handle, both used herein interchangeably. The reactive handle comprises the group on the modifying polypeptide binding moiety that attaches to the linker. In one example embodiment, the reactive handle can perform click chemistry, amide coupling chemistry, crosslinking chemistry, alkylation, or sulfonation chemistry. (See e.g., Nwe, K.; Brechbiel, M. W. Growing Applications of “Click Chemistry” for Bioconjugation in Contemporary Biomedical Research. Cancer Biotherapy and Radiopharmaceuticals, 2009, 24, 289-302.)

In one example embodiment, the bond is chosen to be energetically favorable, preferably increasing binding affinity. The exit vector may be adjusted depending on the linker utilized in the molecules. In one example embodiment, the exit vector is a chemical moiety or bond that facilitates stereochemical protrusion that may further facilitate subsequent coupling, bonding and/or accessibility.

In an embodiment, L is a rigid linker, which may be selected from the group consisting of:

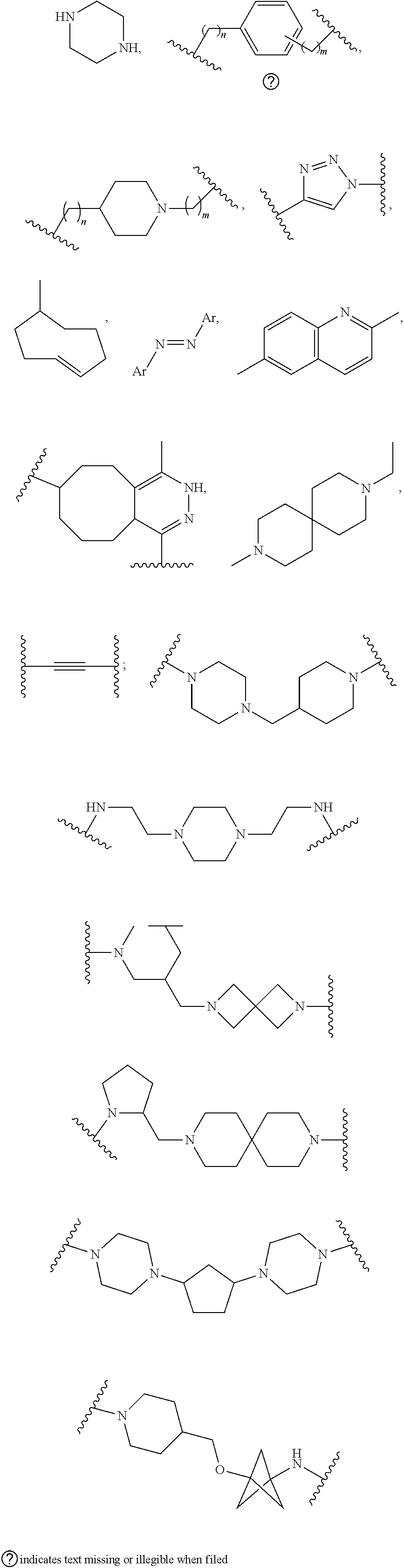

or any combination thereof; and wherein any atom in within a ring may substituted for C, NO, S; the linkers may bond to one or more PEG molecules before bonding to A and optionally B; and m and n may be independently selected from 0 to 6.

In preferred example embodiments, the linker L has one covalent attachment point to a modifying polypeptide binding molecule and two covalent attachment points to the other target binding molecule, or vice versa. A covalent attachment point may be any single, double, triple, or quadruple bond between one component of the BFM/chimeric small molecule and another. In preferred example embodiments, the linker is attached to one modifying polypeptide binding molecule, i.e. A, and the other, i.e. B, according to the formula

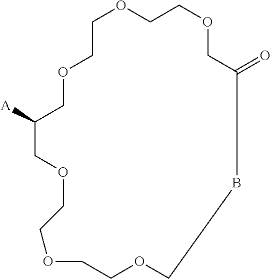

In one example embodiment, the PEG compounds in the previously mentioned linker can be substituted for any linker mentioned herein. In One example embodiment, the previously mentioned linker is optimized for physiochemical properties, such as solubility and/or permeability, and/or pharmacokinetic properties, such as microsomal stability or target binding.

In one example embodiment, the modifying polypeptide binder has an adapter or reactive handle, both used herein interchangeably. The reactive handle comprises the group on the modifying polypeptide binder that attaches to the linker. In one example embodiment, the reactive handle can perform click chemistry, amide coupling chemistry, crosslinking chemistry, alkylation, or sulfonation chemistry.

### Orienting Adaptor

The electrophilic reactive linker may comprise one or more orienting adaptors. In embodiments, an orienting adaptor can be utilized at each instance of a localizing moiety or an activator moiety. In an aspect, an orienting adaptor is appended on different ends of a linker molecule, with an orienting adaptor attached to each polypeptide binding moiety of the electrophilic reactive linker, and optionally, provides an orienting adaptor interconnecting the electrophilic reactive linker on a different end to a polypeptide binding moiety.

In embodiments, the orienting adaptor is a small molecule group that aids in the orienting of the polypeptide binding moieties. In embodiments, the orienting adaptors are chosen so that the small molecule compounds bind in one of their preferred, low-energy conformations. By way of example, when a protein is the substrate, a protein dissipates strain energy through small changes across its degrees of freedom more easily than for the small molecule to adopt an unfavorable conformation by straining its few rotatable bonds. Accordingly, ‘soft’ or low-energy torsion barriers are helpful when designing the small molecule compounds. The preferences can be considered when designing orienting molecules between aryl rings. Anisoles (ArOCH₂R) and anilines (ArNHR) prefer coplanar conformations, alkylaryls (ArCH₂R), arylsulfonamides and arylsulfones prefer a perpendicular conformation. Orienting adaptor atoms control both distance and direction. See, e.g. Brameld et al. J. Chem. Inf. Model. 2008, 48, 1-24.

In some instances, the orienting adaptors can be referred to in embodiments as exit vectors. Exit vector parameters can be identified in part based on average orientation of a substituent attached to a variation point which can be generated using chemoinformatics software. An exit vector may comprise outgoing bonds from a chemical moiety. In certain embodiments, the bond is chosen to be energetically favorable, preferably increasing binding affinity. The orienting adaptor may be represented in certain embodiments with the linker, and may be adjusted depending on the linker utilized in the multifunctional molecules. In embodiments, the orienting adaptor is a chemical moiety or bond that facilitates stereochemical protrusion that may further facilitate subsequent coupling, bonding and/or accessibility. In embodiments, the first and second orienting adaptors are provided as bonds on the linker, providing conformation of attachment between the linker and the activator moiety and/or the localizing moiety.

In example embodiments, the orientation adaptor comprises:

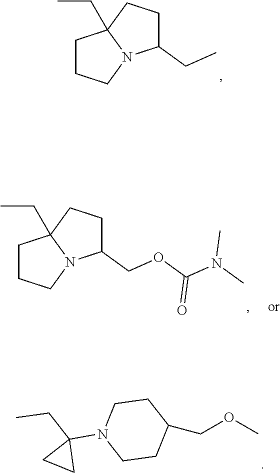

In embodiments, the first and second orienting adaptor, when present, are independently selected from Table A (Orienting Adaptor Table).

TABLE A

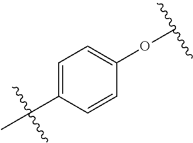 [SMILES: *Oc1ccc(*)cc1]

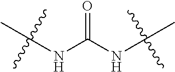 [SMILES: *NC(=O)N*]

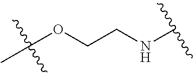 [SMILES: *NCCO*]

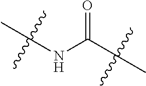 [SMILES: *NC(*)=O]

—O—

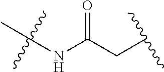 [SMILES: *CC(=O)N*]

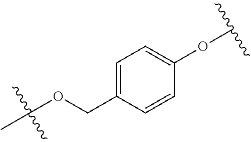 [SMILES: *OCc1ccc(O*)cc1]

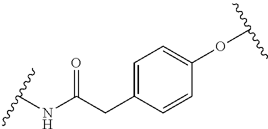 [SMILES: *NC(=O)Cc1ccc(O*)cc1]

—NH—

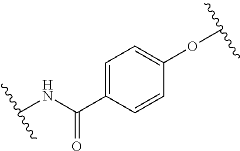 [SMILES: *NC(=O)c1ccc(O*)cc1]

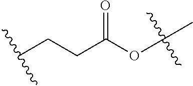 [SMILES: *CCC(=O)O*]

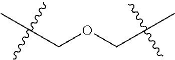 [SMILES: *COC*]

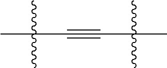 [SMILES: *C#C*]

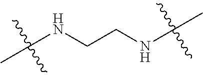 [SMILES: *NCCN*]

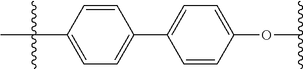 [SMILES: *Oc1ccc(-c2ccc(*)cc2)cc1]

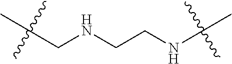 [SMILES: *CNCCN*]

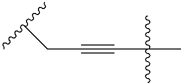 [SMILES: *C#CC*]

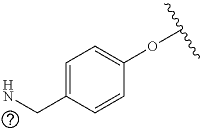

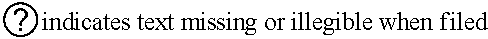

### Chimeric Small Molecules

In one example embodiment, the chimeric small molecule is according to the general formula:

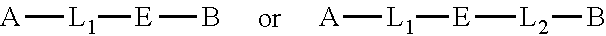

wherein A is a modifying polypeptide binding moiety, B is a target binding moiety, L₁ and L₂ are each a linker, and E is an electrophilic reactive linker. As described herein, a chimeric small molecule and bifunctional molecule can be used interchangeably.

The embodiments of these formulae may be useful in, but not necessarily limited, to situations where the modifying polypeptide binding moiety would otherwise inhibit or interfere with the ability of the modifying polypeptide bound by the modifying polypeptide binding moiety to modify the target substrate bound by the target binding moiety.

The electrophilic reactive linker of the chimeric small molecule may be designed to react with a moiety on the modifying polypeptide, for example, on an amino acid of the modifying polypeptide. The electrophilic reactive linker can be advantageously designed to react with a moiety in proximity to the binding site of the modifying polypeptide binding moiety on the modifying polypeptide. The reaction of the electrophilic reactive linker with a moiety on the modifying polypeptide, for example, a nucleophilic linker of an amino acid disposed on the protein, can allow the labeling or binding of the modifying polypeptide with the target binding moiety. Such binding of a target binding moiety to the modifying polypeptide can generate a reprogrammed modifying polypeptide that can modify a target substrate, including a target substrate other than the naturally occurring substrate of that modifying polypeptide. Accordingly, in an example embodiment, the modifying polypeptide binding moiety binds to the modifying polypeptide and is chosen based on the binding pocket and availability of amino acid side chains in proximity to the binding pocket that may be used in a reaction with the electrophilic reactive linker of the targeting chimera.

The modifying polypeptide binding moiety may bind to a modifying polypeptide and may be specific to one or more modifying polypeptides. In an embodiment, the modifying polypeptide binding moiety can further comprise a bio-orthogonal group. The modifying polypeptide binding moiety, as detailed further herein, can be selected to have a half-life shorter than the half-life of the modifying polypeptide. In an example embodiment, the modifying polypeptide binding moiety binds to a phosphatase, a ubiquitinase, a deubiquitinase, an acetyltransferase, deactylase, methyltransferase, demethylase, or glycosyltransferase.

The target binding moiety can be specific for one or more targets of interest. In one example embodiment, the target of interest is a macromolecule, e.g., a protein. The target binding moiety can bind the target of interest (target substrate), thereby bringing the modifying polypeptide into proximity to the target of interest. In an embodiment, the target of interest may advantageously be a non-cognate substrate of the modifying polypeptide. The target of interest may be a pathogenic or oncogenic target.

### Half-Life

In an example embodiment, where the chimeric small molecule is being used to label the surface of the modifying polypeptide with a target binding moiety, the modifying polypeptide binding moiety may be chosen in part based on its half-life. In one example embodiment, the modifying polypeptide binding moiety may be chosen based in part on its half-life relative to the half-life of the modifying polypeptide. In an embodiment, the half-life of the modifying polypeptide binding moiety is 2, 3, 4, or 5 times shorter than that of the modifying polypeptide. Without being bound by a particular theory, design of a chimera small molecule with a half-life of the modifying polypeptide binding moiety shorter than that of the modifying polypeptide may allow for desirable reaction kinetics when the modifying polypeptide is labeled with the modifying polypeptide binding moiety via the electrophilic reactive linker. The half-life of the modifying polypeptide binding moiety and the modifying polypeptide generally relates to the time required for the concentration of the modifying polypeptide binding moiety or modifying polypeptide to decrease to half of its initial concentration. In one example embodiment, the half-life may measure the time it takes to degrade half of the molecules initially measured in a sample, which may comprise a cell, cells, tissue, organoid, or mammal, for example. In one example embodiment, the half-life of the modifying polypeptide and the modifying polypeptide binding moiety is measured in the same or similar conditions, for example, in a same cell type, tissue, or organism. In one example embodiment, the measurement of half-life can be measured in a same sample or system that has a particular phenotype, genotype, disease or condition to be studied, treated and/or evaluated.

Measurement of the half-life of the modifying polypeptide binding moiety may be determined, for example, by dissociation t_{1/2} or receptor occupancy t_{1/2}, describing the average time needed to liberate half of the initially occupied receptors under conditions in where association of the protein binding moiety or its rebinding can take place. Dissociation that requires a receptor conformational change or binding pocket size may play a factor in the residence time and can be considered when selected the protein binding moiety. See, e.g., Roskoski R Jr. Classification of small molecule protein kinase inhibitors based upon the structures of their drug-enzyme complexes. Pharmacol Res. 2016; 103:26-48. doi:10.1016/j.phrs.2015.10.021.

The time a compound resides on its target, e.g., residence time, may be used. See, Willemsen-Seegers N, Uitdehaag J C M, Prinsen M B W, et al. Compound Selectivity and Target Residence Time of Kinase Inhibitors Studied with Surface Plasmon Resonance. J Mol Biol. 2017; 429(4):574-586. doi:10.1016/j.jmb.2016.12.019, for discussion and identification of residence time and kinetic parameters of exemplary kinase binding moieties, incorporated herein in its entirety, and in particular Table 1,3A-3B, 4A-4C, S3 and S4, for teachings to tyrosine kinase inhibitors, EGFR inhibitors, ponatinib to a variety of kinases, particular kinases and their associated inhibitors, Aurora A and B kinase inhibitors, and P13k lipid kinase inhibitors. Elimination half-life may also be utilized alone or in conjunction with residence time evaluation. Additional pharmacodynamics and pharmacokinetics may also be considered in the evaluation of half-life for the modifying polypeptide binding moiety. Half-life may be modeled. See, e.g. Callegari D, Lodola A, Pala D, et al. Metadynamics Simulations Distinguish Short- and Long-Residence-Time Inhibitors of Cyclin-Dependent Kinase 8 [published correction appears in J Chem Inf Model. 2017 Feb. 27; 57(2):386]. J Chem Inf Model. 2017; 57(2):159-169. doi:10.1021/acs.jcim.6b00679, incorporated herein by reference.

The measurement of half-life of the modifying polypeptide, approaches measuring half-life such as mass spectrometry-based proteomics such as SILAC (stable isotope labeling by amino acids in cell culture)-based proteomics, see, e.g. Matheison et al., Nature Communications volume 9, Article number: 689 (2018), may be used. High throughput proteomics may be used to estimate a modifying polypeptide half-life in a particular tissue and/or cell, or further predictive modeling may be used to predict such modifying polypeptide half-life in tissue from cellular properties, see, e.g. Rahman M, Sadygov R G Predicting the protein half-life in tissue from its cellular properties. PLOS ONE 12(7): e0180428. doi.org/10.1371/journal.pone.0180428 (2017).

The modifying polypeptide binding moiety can be chosen based on the target substrate and the modification to that substrate desired. Advantageously, the modifying polypeptide binding moiety can be an activator or inhibitor of the modifying polypeptide. A modifying polypeptide binding moiety may be chosen based on high modifying polypeptide abundance in a target cell; modifying polypeptides with high activity at lower concentrations, e.g. nanomolar activity; available crystal structure and characterization of the modifying polypeptide active; modifying polypeptide binding moieties with low residence time; the ability of the modifying polypeptide binding moiety of the chimeric small molecule to accommodate a bio-orthogonal group, e.g. a small biorthogonal handle, without affecting binding potency and/or residence time; modifying polypeptides with a high density of amino acids with nucleophilic side chains, e.g. serines/threonines/tyrosines/lysines close to the binding pocket; and/or whether the labeling of the modifying polypeptide would interfere with its enzymatic activity, which may be based on experimental data and/or modeling. Linker length on the chimeric small molecule may be tuned, allowing modification, e.g. phosphorylation, with increased distance from binding pocket, allowing modification to be targeted to locations, for example, amino acid residues, farther away from the binding pocket. For example, a longer linker length can be utilized when a bioconjugation reaction is desirable further away from a binding pocket but optimized for a length that still allows the target binding moiety, once bound to a target substrate in close proximity to the binding pocket of the modifying polypeptide. Tuning linker length may also include a level of flexibility or rigidity depending on desired configuration of the target binding moiety for modifications of amino acid residues. A shorter linker length may allow for modification within the binding pocket which may desirable for some applications.

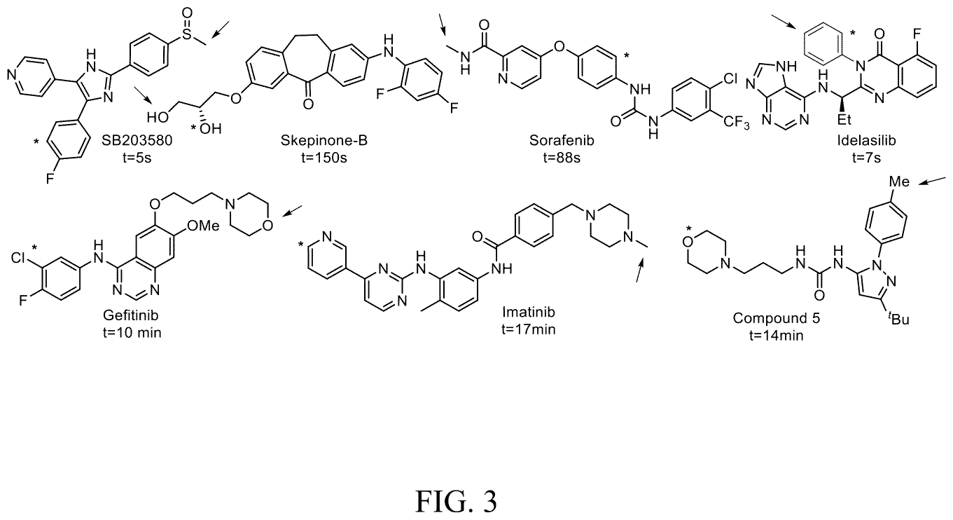

In an example embodiment, the modifying polypeptide binding moiety is an allosteric modulator. Considerations in selecting a modifying polypeptide binding moiety may include allosteric signaling, which may include changes associated with networks of non-covalently interacting protein residues, conformational selection, and induced fit with both spatial and temporal aspects. In one example embodiment, the modifying polypeptide binding moiety may be an allosteric activator or inhibitor of the modifying polypeptide. Allosteric activators or inhibitors may be discovered computationally. In one example method, high quality drug targets are acquired. Then allosteric site prediction is performed using methods such as perturbation response scanning (PRS) combined with all-atom molecular dynamics (MD) and dynamic residue networks (DRN). Allosteric modulators are then identified using methods such as homology modeling, docking, or essential dynamics. An illustration of this process can be found in FIGS. 2 and 3 of Amamuddy S., et al. Integrated Computational Approaches and Tools for Allosteric Drug Discovery. 21 IJMS, 847 (2020), incorporated herein by reference.

Modifying polypeptide binding moieties may be chosen based on the type of desired modification to be made by the modifying polypeptide, for example, post-translational modification of the target substrate. In one example embodiment, the modifying polypeptide binding moiety is capable of binding an modifying polypeptide that phosphorylates a target, thus the type of modifying polypeptide may be chosen for this desired modification of a target substrate. Post-translational modification (PTM) is one type of modification performed. Accordingly, post-translational modification phosphatase, a ubiquitinase, a deubiquitinase, an acetyltransferase, deactylase, methyltransferase, demethylase, and/or glycosyltransferase are a set of binding moieties envisaged for use in the present invention.

In an example embodiment, the modifying polypeptide binding moiety provides a modification to an amino acid, see, e.g. for example Table 1 of Karve et al., Journal of Amino Acids Volume 2011, Article ID 207691, 13 pages, DOI: 10.4061/2011/207691, incorporated herein by reference. Karve et al. summarizes some post-translational modifications and their importance in various diseases as well as normal development. Karve et al. assesses, phosphorylation of amino acids, and is incorporated specifically for the phosphorylation modifications detailed therein.

The reaction of the electrophilic reactive linker with a moiety on the modifying polypeptide, for example, a nucleophilic group disposed on the modifying polypeptide, can allow the labeling or binding of the modifying polypeptide with the target binding moiety. Such binding of a target binding moiety to the modifying polypeptide can generate a reprogrammed modifying polypeptide that can modify a target substrate. Accordingly, in an example embodiment, the modifying polypeptide binding moiety binds to the modifying polypeptide and is chosen based on the binding pocket and availability of amino acid side chains in proximity to the binding pocket that may be used in a reaction with the electrophilic reactive linker of the targeting chimera.

### Kinase Binding Moiety

The chimeric small molecule according to the general formula A-L₁-E-B or A-L₁-E-L₂-B comprises a kinase binding moiety A that may comprise any molecule capable of non-covalent bonding to a kinase. The kinase binding moiety, also referred interchangeably herein as a kinase binder, of the chimeric small molecule may target one or more different kinases, or one or more locations on the kinase.

A kinase belongs to a family of phosphotransferases and phosphorylates a substrate by transferring the gamma phosphate of ATP onto hydroxyl groups of the substrate. Substrates may comprise lipids, sugars or amino acids. The kinase binding moiety may be any molecule capable of binding to a kinase. Some kinase binding molecules are known to activate a kinase upon binding while others are known to inhibit a kinase upon binding. In one example embodiment, the kinase binding moiety is a kinase activator. In an example embodiment, the kinase binding moiety is a kinase inhibitor. However, binding of the kinase, rather than its inhibitory or activation behavior of the kinase binding moiety, is the primary objective as the design of the targeting chimeras generates a kinase labeled with a target binding moiety, with the kinase binding moiety utilized for initial binding to the kinase to allow for generation of a repurposed kinase labeled with target binding moiety rather than use of the kinase binding moiety for kinase activation or inhibition properties.

The chimeric small molecule is preferably designed such that one or more nucleophilic groups disposed on the protein, e.g. kinase, readily reacts with the electrophilic reactive linker of the chimeric small molecule. The moiety comprising the nucleophilic group of the protein may be an amino acid side chain. Thus, the kinase binding moiety can be chosen based on the binding pocket and availability of amino acid side chains in proximity to the binding pocket that can be used in a reaction with the electrophilic reactive linker of the targeting chimera. Further design of exit vectors that are comprised on the kinase binder or linker of the chimeric small molecule may further allow for desired configuration of the kinase binding moiety at the protein binding site.

### Example Kinase Binding Moiety

In an example embodiment, the kinase binding moiety is a kinase activator moiety. The kinase activator moiety can be a small molecule or compound that activates a kinase. As used herein, a kinase is an enzyme that adds a phosphate group to another molecule, typically an amino acid of a protein substrate. An activator of a kinase enhances such phosphorylation activity. In one example embodiment, the kinase activator moiety promotes an active conformation of an enzyme, in one aspect, trough binding interactions with regulatory subunits. See, e.g. Zorn et al Nat Chem Biol. 2010 March; 6(3):179-188; doi: 10.1038/nchembio.318. The kinase may act on the amino acid serine, threonine, tyrosine, or a combination thereof.

Activator moieties can be identified from activators known in the art. The activators may be a derivative of activators known in the art, and may comprise fewer or additional functional groups that still permit activator activity, but may enhance or facilitate the desired formation, conformation or attachment sites for the chimeric small molecules described herein. Exemplary modifications may include derivatives for increase solubility, charge, functionality for use with an orienting adaptor or linker, detailed elsewhere in the specification.

In one embodiment, the kinase binding moiety is a kinase inhibitor. A kinase inhibitor (KI) is generally designed to bind with a highly conserved Asp-Phe-Gly (DFG) motif of a kinase. KIs can be classified by the conformation of the DFG binding site. Type I bind to the active, DFG-Asp-in, conformation while Type II inhibitors bind to the inactive, DFG-Asp-out, conformation. Further consideration of kinase inhibitors include competition with ATP-binding, which may include mimicking the hydrogen binding interactions normally formed by the adenosine ring of ATP, or the mechanism of inhibition such as reversible binding or irreversible covalent bonding. See e.g. (Gross et al. J Clin Invest. 2015; 125(5):1780-1789).

A consideration of kinase inhibitor design has been the degree of specificity to a particular kinase. While the assumed advantage has been for more specificity, kinase inhibitors with a low degree of specificity for a particular kinase facilitates the recruitment of many types of kinases. A promiscuous kinase inhibitor is advantageous as the kinase is a vehicle for the modification of the target substrate.

In one embodiment, the protein binding moiety is a promiscuous kinase inhibitor (PKI). A promiscuous kinase inhibitor refers to a molecule that binds to more than one kinase. A promiscuous kinase inhibitor is a molecule that has binding specificity to a binding pocket with high conservation across kinases. A promiscuous kinase inhibitor may bind to 2, 3, 4, 5 or more different kinases. In one example embodiment, the promiscuous kinase inhibitor is an ATP-competitive kinase inhibitor. In one example embodiment, the PKIs target one or more kinases selected from PDGFRA, PDGFRB, KIT, CSFIR, DDR1, DDR2, MEK5, and YSK4. See, e.g. Seeliger, M. A., et al. “What Makes a Kinase Promiscuous for Inhibitors?” Cell Chem. Biol., 26 (3), 2019; 390-399. For example, the kinase inhibitor imatinib can inhibit c-KIT, PDGFR-alpha and BCR-ABL kinases (see, e.g. Iqbal N, Iqbal N. Imatinib: a breakthrough of targeted therapy in cancer. Chemother Res Pract. 2014; 2014:357027. doi: 10.1155/2014/357027. Epub 2014 May 19); similarly, sunitinib, sorafenib, and cabozantinib are also known for their promiscuous activity and are provided as non-limiting examples of promiscuous kinase inhibitors. In one example embodiment, the PKI is modified to contain a bio-orthogonal group.

In one example embodiment, the protein binding moiety is a kinase binding moiety. Example kinases that may be bound by the chimeric small molecules of the present invention include, but are not limited to, PK, PKC, AMPK, MAPK, EGFR, FGFR, NGFR, TrkA, ABL, BCKDK, CDK, PI3K, VEGFR, BRAF, MEK, AKT, ALK, BTK, FLT3, JAK2, AURKA, c-MET, DDR, FKBP, INSR, IKK, JNK, mTOR, PAK, PDK1, PDK2, PTK2/FAK, pyruvate kinases, RAC-α, RIPK, TYK2, SHP, aPKC, NOP, u (mu) opioid receptor, 8 (delta) opioid receptor, UMPK, SphK, or GSK-3.

### ABL Binding Moiety

In one example embodiment, the protein binding moiety is an ABL kinase binding moiety. Abelson kinases (ABL) is a ubiquitously expressed, nonreceptor tyrosine kinase which plays a key role in cell differentiation and survival. Simpson, et al., J. Med. Chem. 2019 62, 2154-2171. ABL tyrosine kinase can be found in the nucleus, cytoplasm, and mitochondria. ABL proteins are normally under well-orchestrated regulation. However, chromosome translocations that join the ABL genes with genes coding for other proteins give rise to various fusion proteins that are prone to dimerization (or oligomerization) and autophosphorylation. Consequently, ABL kinase becomes constitutively active, leading to myeloproliferative disorders. In one example embodiment, one of the ABL kinase binding moieties as detailed herein is used with a target binding moiety as described herein in a chimeric small molecule.

In one example, embodiment, the ABL kinase binding moiety is an ABL kinase activator. In one example embodiment, the c-Abl Kinase activator is (5-[3-(4-fluorophenyl)-1-phenyl-1H-pyrazol-4-yl]-2,4-imidazolidinedione or 5-(1,3-diaryl-1H-pyrazol-4-yl) hydantoin):

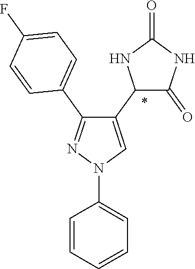 [SMILES: O=C1NC(=O)C(c2cn(-c3ccccc3)nc2-c2ccc(F)cc2)N1]

(DPH) as described in Yang et al., “Discovery and Characterization of a Cell-Permeable, Small-Molecule c-Abl Kinase Activator that Binds to the Myristoyl Binding Site, Chem. & Biol., 18, 177-186, Feb. 25, 2011.

In one example embodiment, the c-Abl kinase activator can be selected from

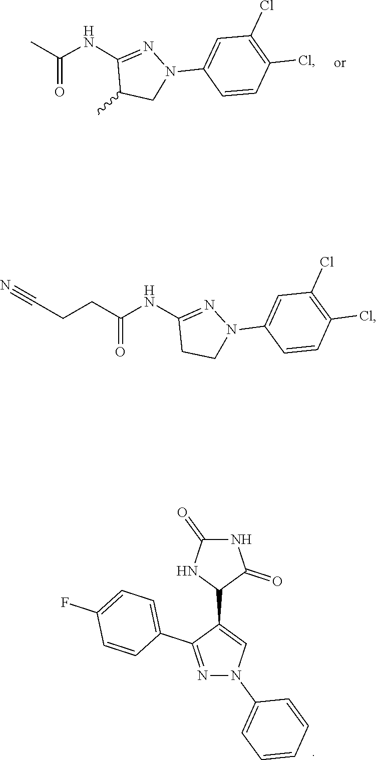

which showed in vivo activation of c-Abl in Simpson, G. L., et al. “Identification and Optimization of Novel Small C-Abl Kinase Activators Using Fragment and HTS Methodologies.” J. Med. Chem. 2019, 62 (4), 2154-2171. The novel aminopyrazoline small molecule activators described in Simpson et al. at Table 6, are specifically incorporated herein by reference.

In one example embodiment, the c-Abl kinase activator is

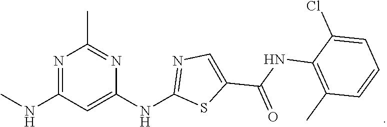 [SMILES: CNc1cc(Nc2ncc(C(=O)Nc3c(C)cccc3Cl)s2)nc(C)n1]

In one example embodiment, the c-Abl kinase binding moiety is a (5-[3-(4-fluorophenyl)-1-phenyl-1H-pyrazol-4-yl]-2,4-imidazolidinedione or 5-(1,3-diaryl-1H-pyrazol-4-yl) hydantoin) (DPH) derivative according to the formula

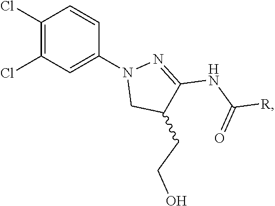

wherein R is

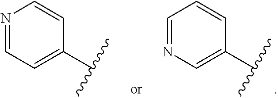 [SMILES: *c1ccncc1 ; *c1cccnc1]

In one example embodiment, the DPH is functionalized:

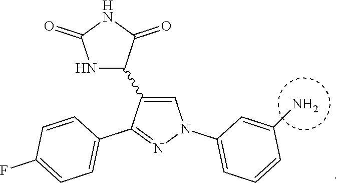 [SMILES: Nc1cccc(-n2cc(C3NC(=O)NC3=O)c(-c3ccc(F)cc3)n2)c1]

In one example embodiment, the ABL kinase activator is

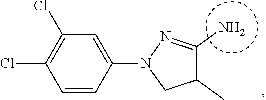

wherein the dashed circle identifies the attachment for the orienting adaptor and/or linker. The functional groups depicted in the dashed circle of the ABL kinase activator can be utilized in methods for attaching a linker and orienting adaptor prior to attachment to the protein binding moiety.

Activator moieties may be functionalized for methods of attaching orienting adaptor and linker. ABL kinase activator parent molecule DPH can be functionalized for methods of attaching orienting adaptor and linker. Exemplary molecules may be:

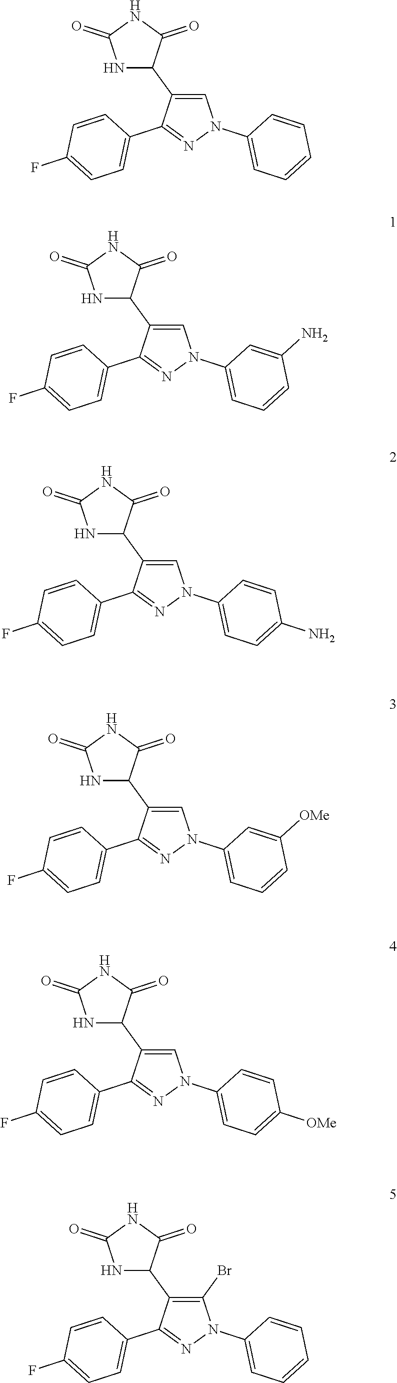 [SMILES: O=C1NC(=O)C(c2cn(-c3ccccc3)nc2-c2ccc(F)cc2)N1 ; Nc1cccc(-n2cc(C3NC(=O)NC3=O)c(-c3ccc(F)cc3)n2)c1 ; Nc1ccc(-n2cc(C3NC(=O)NC3=O)c(-c3ccc(F)cc3)n2)cc1 ; COc1cccc(-n2cc(C3NC(=O)NC3=O)c(-c3ccc(F)cc3)n2)c1 ; COc1ccc(-n2cc(C3NC(=O)NC3=O)c(-c3ccc(F)cc3)n2)cc1 ; O=C1NC(=O)C(c2c(-c3ccc(F)cc3)nn(-c3ccccc3)c2Br)N1]

Once functionalized, the orienting adaptor and linker can be added, either sequentially, or at once, with the orienting adaptor and linker added as one molecule. Exemplary molecules are provided below, with the R group representing the protein binding moiety.

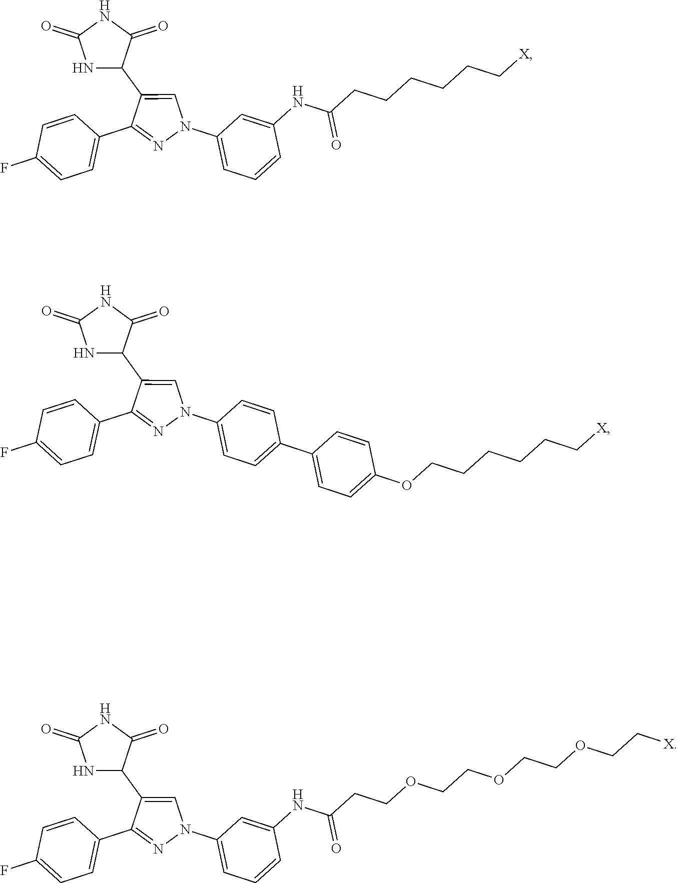

Optionally, more than one activator moiety can be attached to the protein binding moiety. In each instance, the activator moiety identified can be functionalized as described herein for methods of attaching a linker and orienting adaptor prior to attachment to the protein binding moiety, for example, utilizing the functional groups depicted in a dashed circle.

In an example embodiment, the Abl kinase activator is DPH or dihyropyrazol activator. An exemplary molecule may comprise

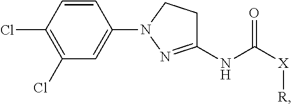

wherein X is (CH2)n, which may be substituted, for example with one or more of amide, acetal, aminal, amine, alkyl, ether, hydrocarbyl, and derivatives thereof, or other groups as described elsewhere herein. In one example embodiment, n is 0 to 20, more preferably n is 1 to 10, or 2 to 7, and R is

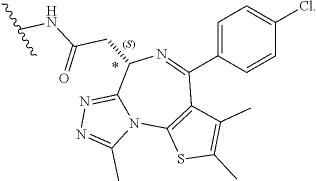

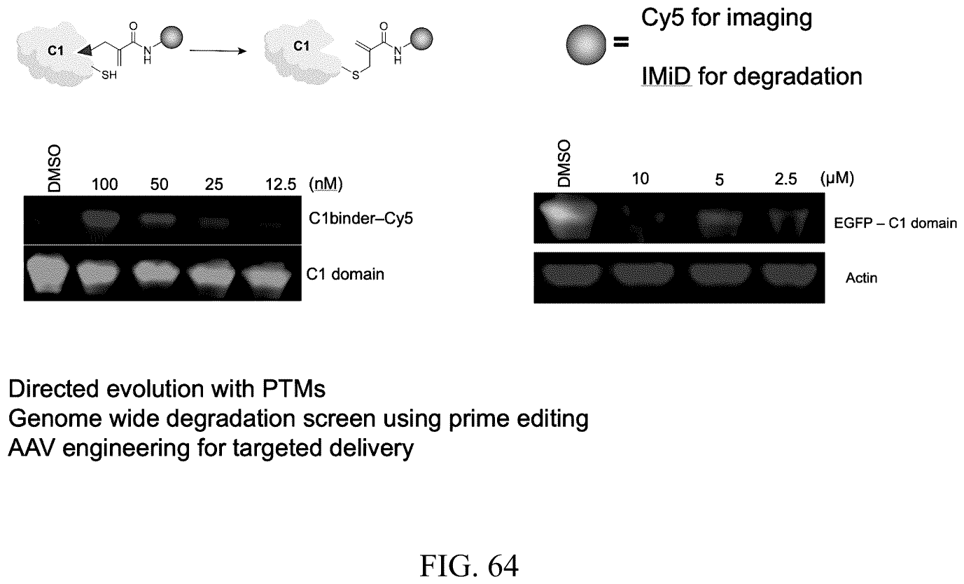

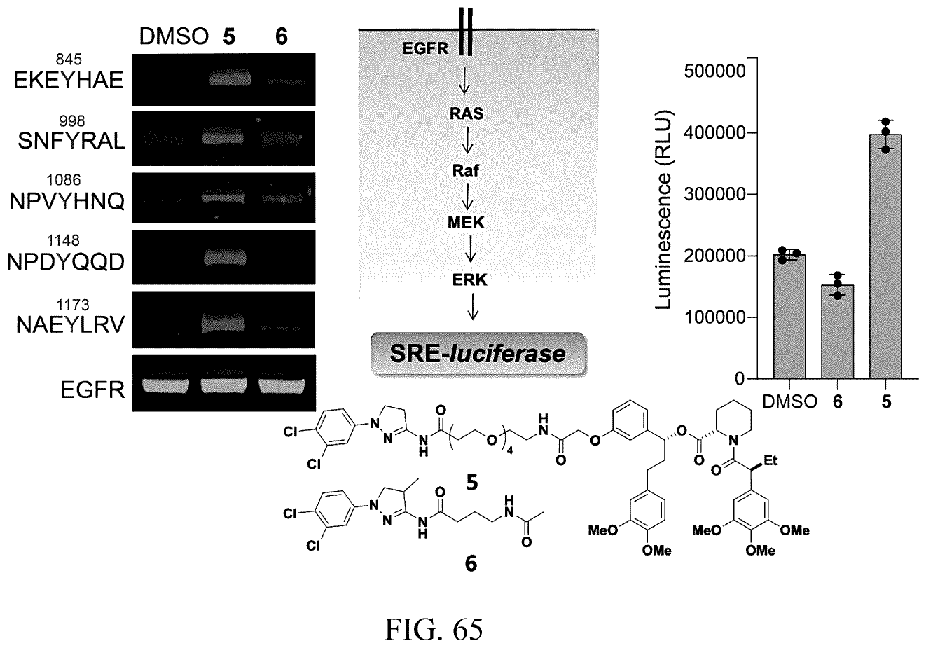

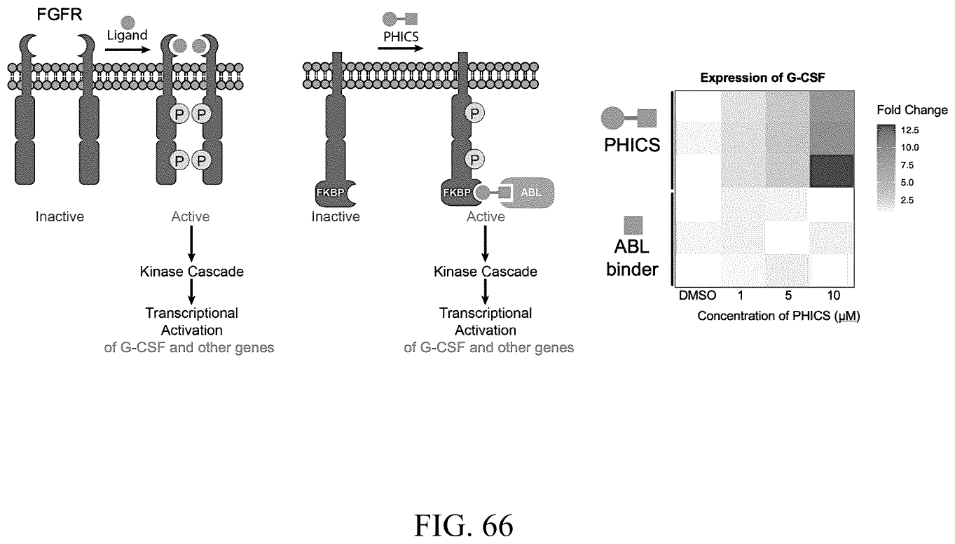

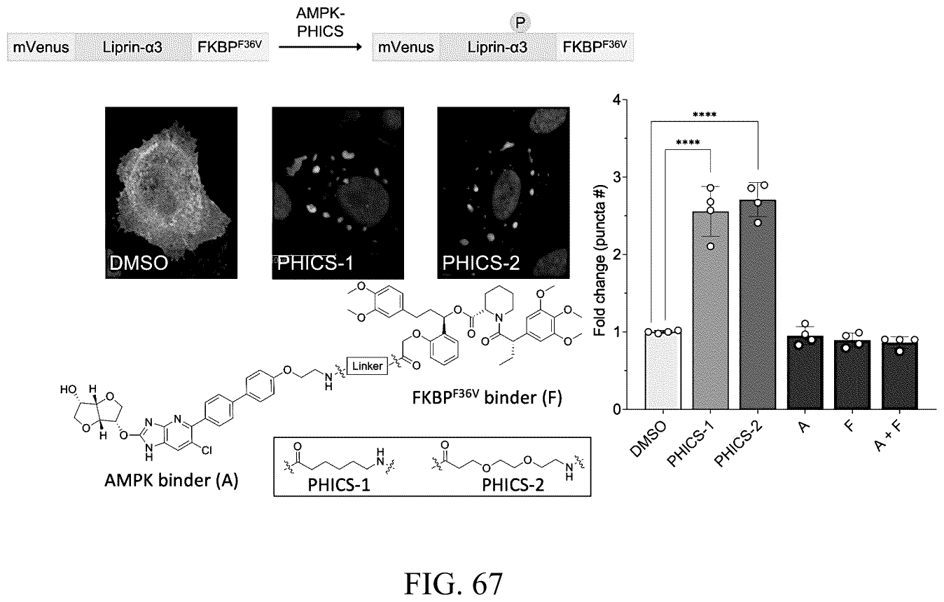

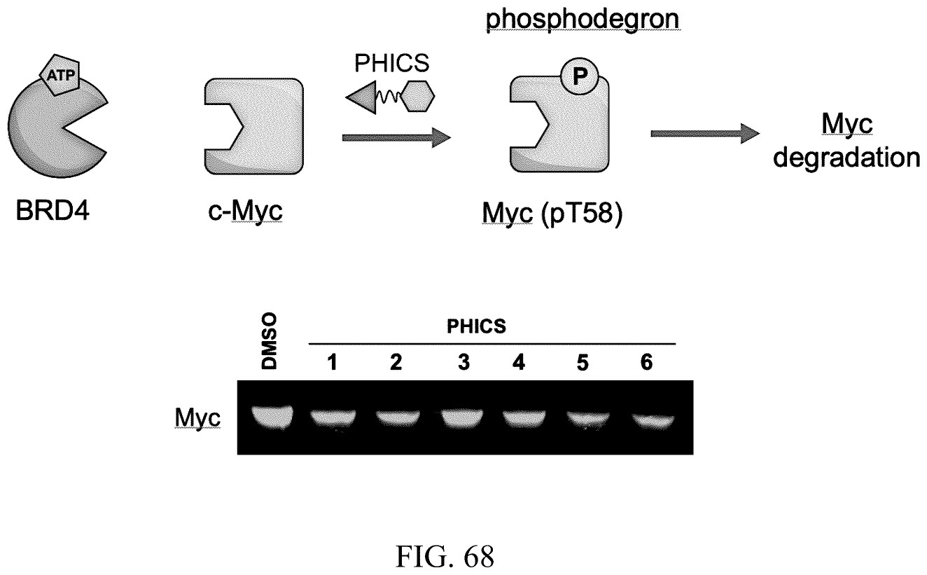

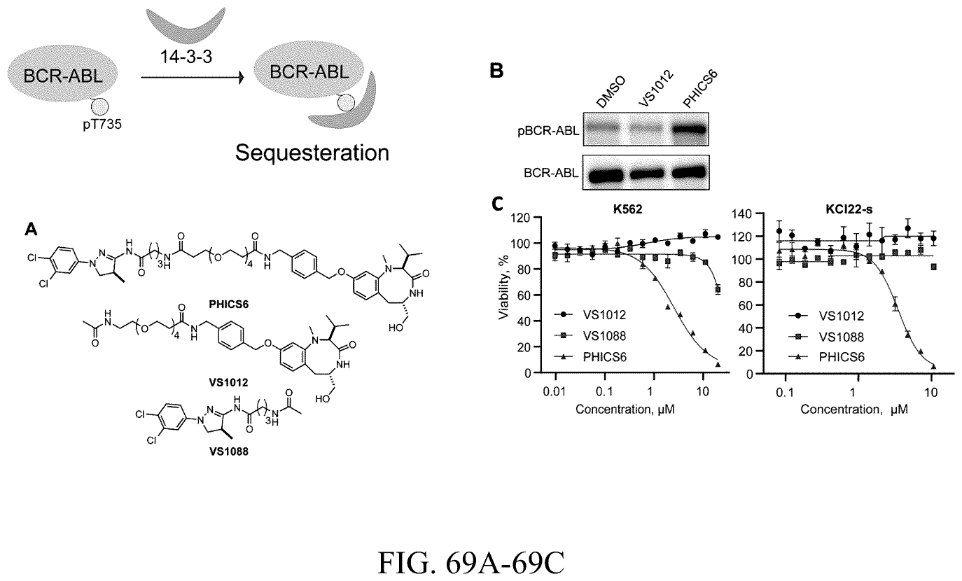

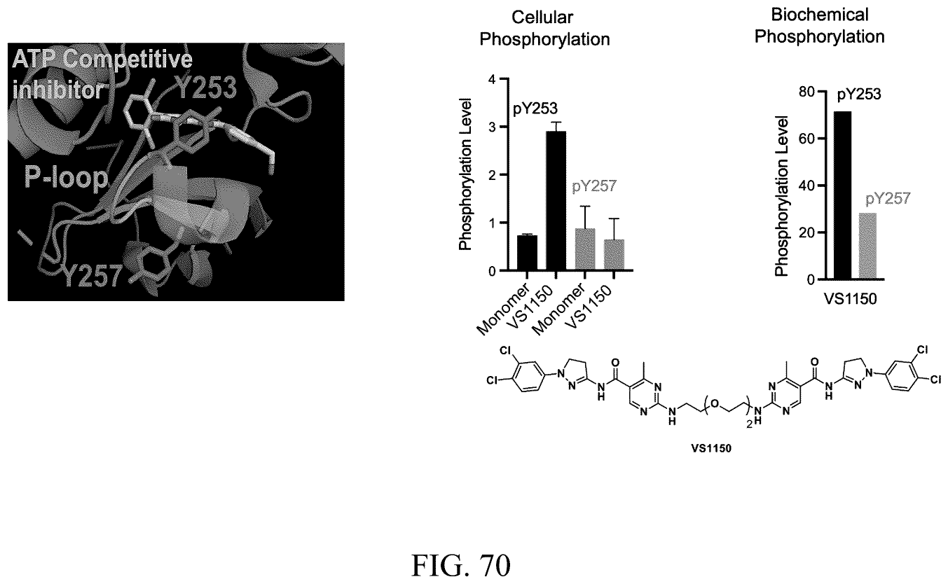

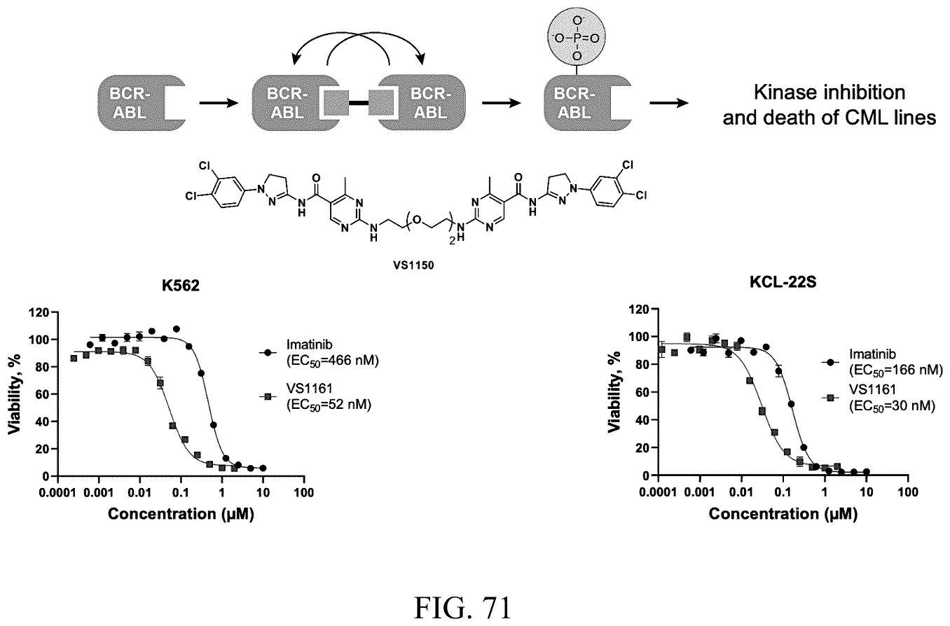

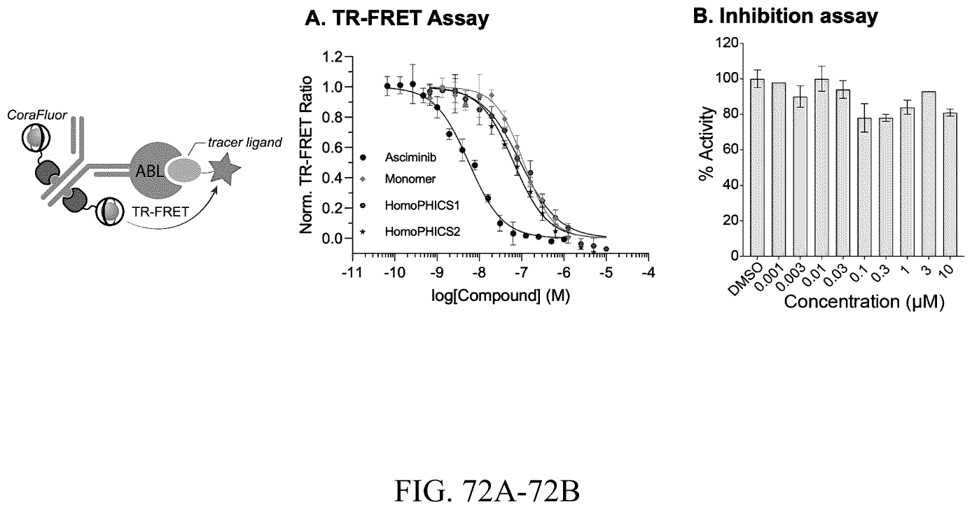

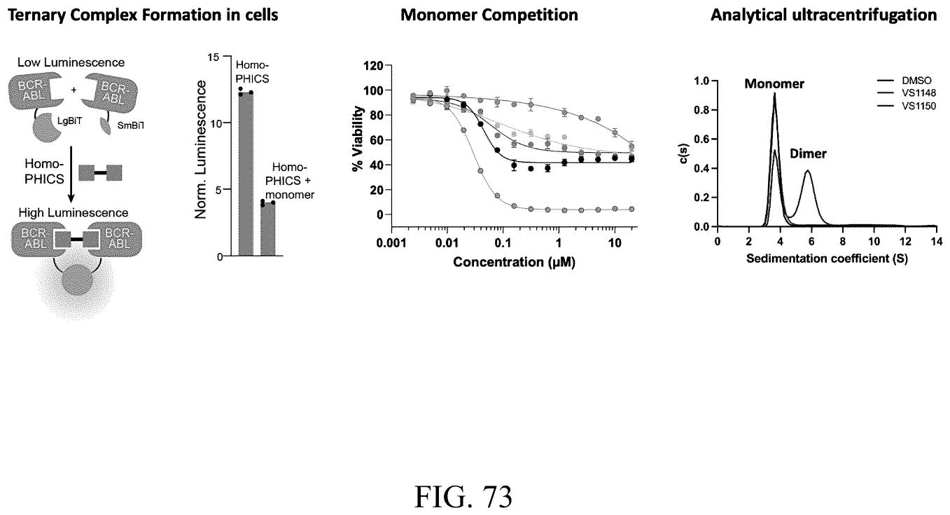

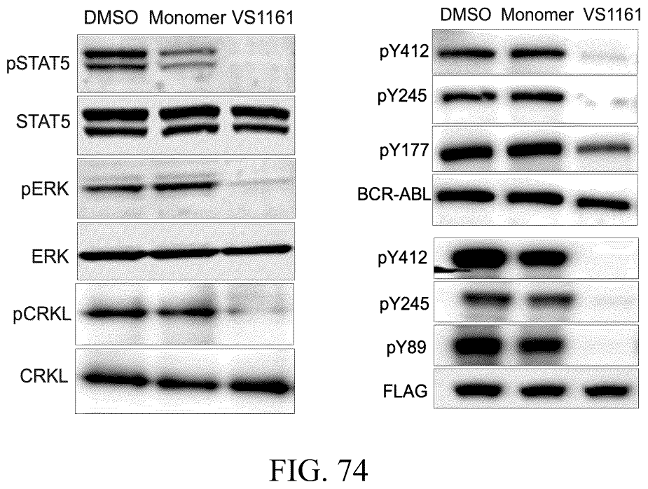

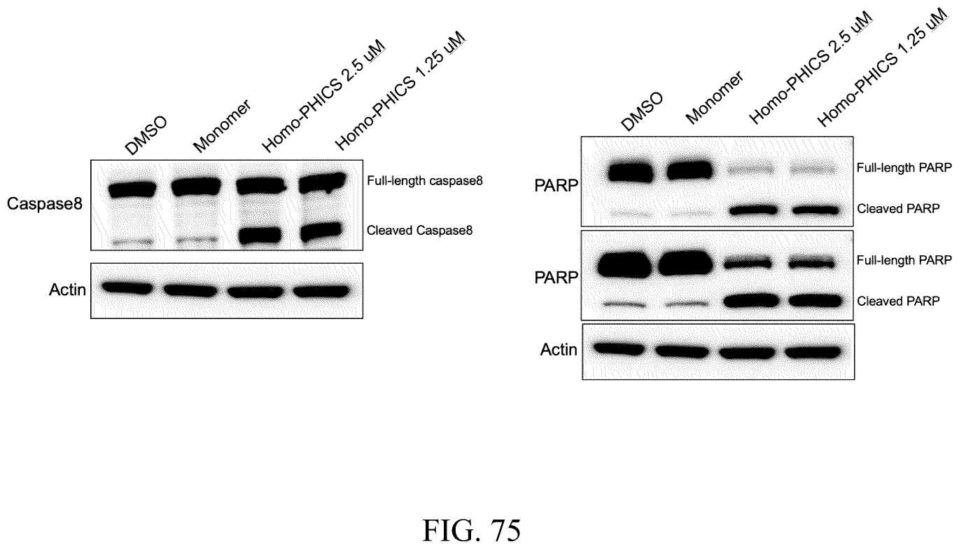

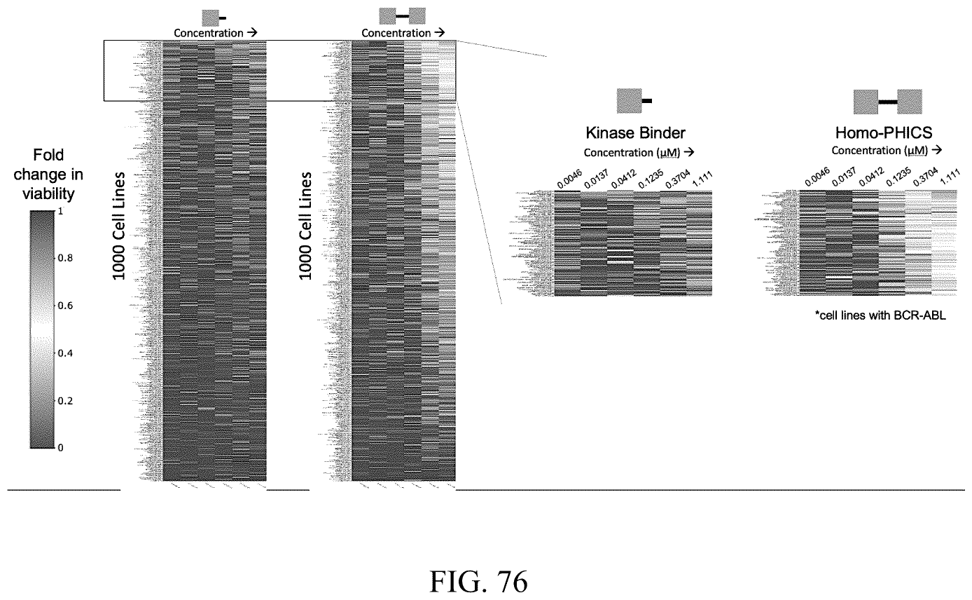

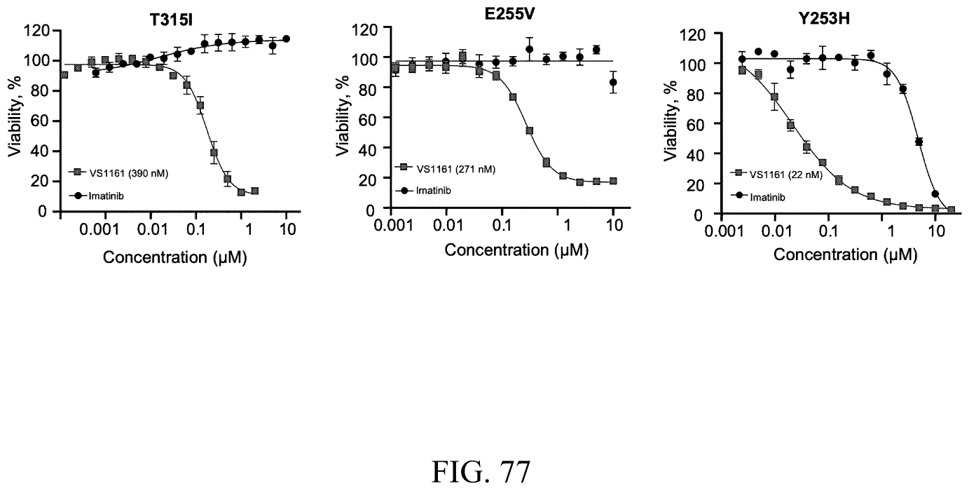

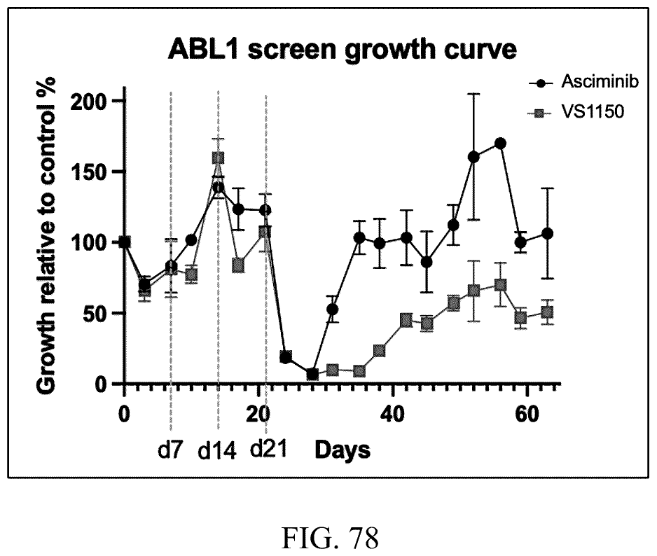

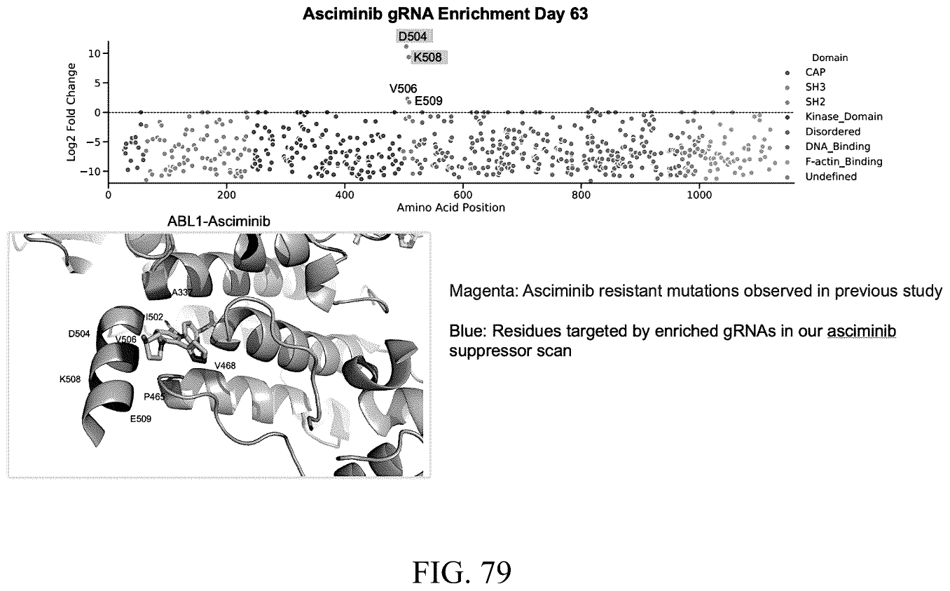

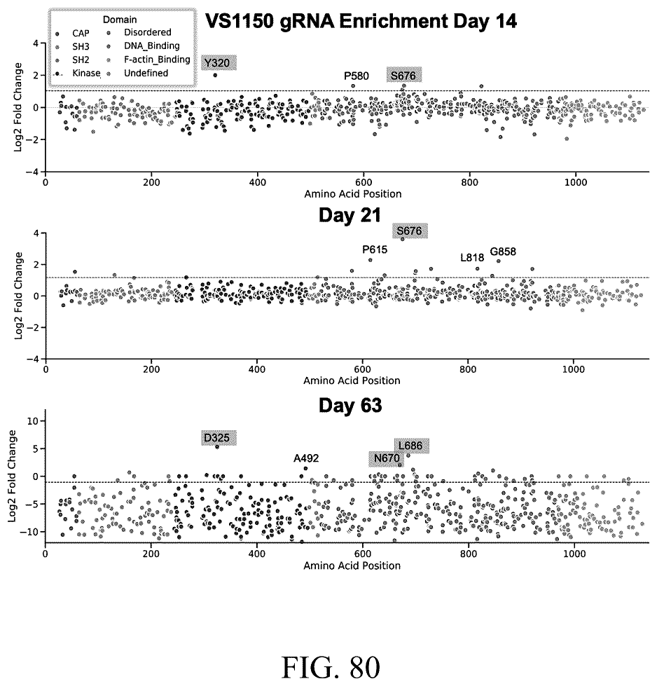

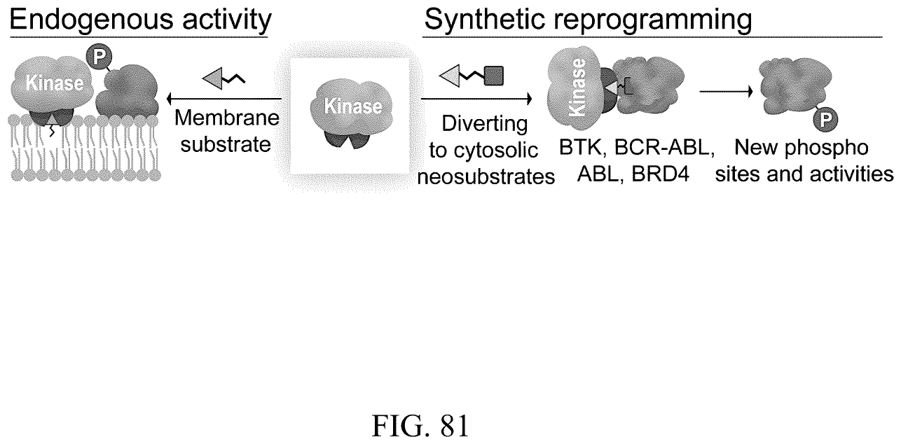

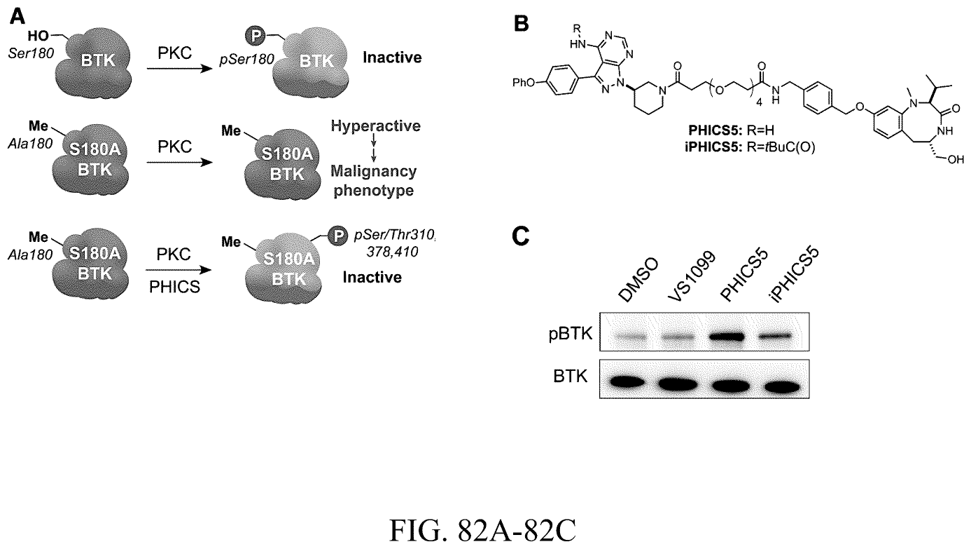

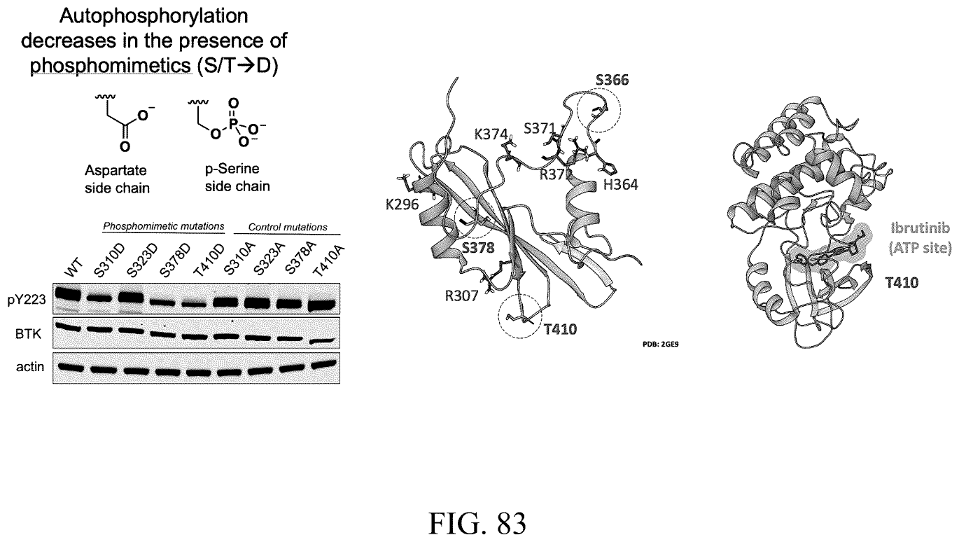

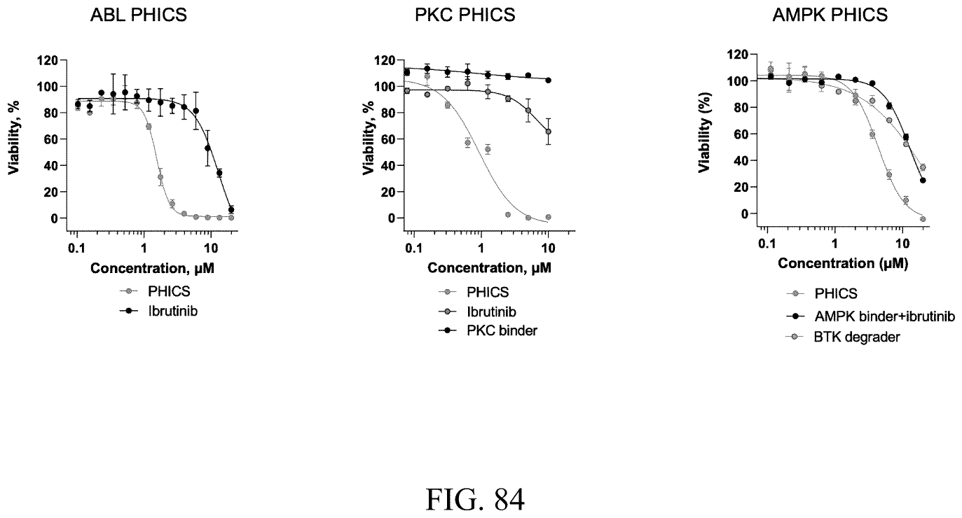

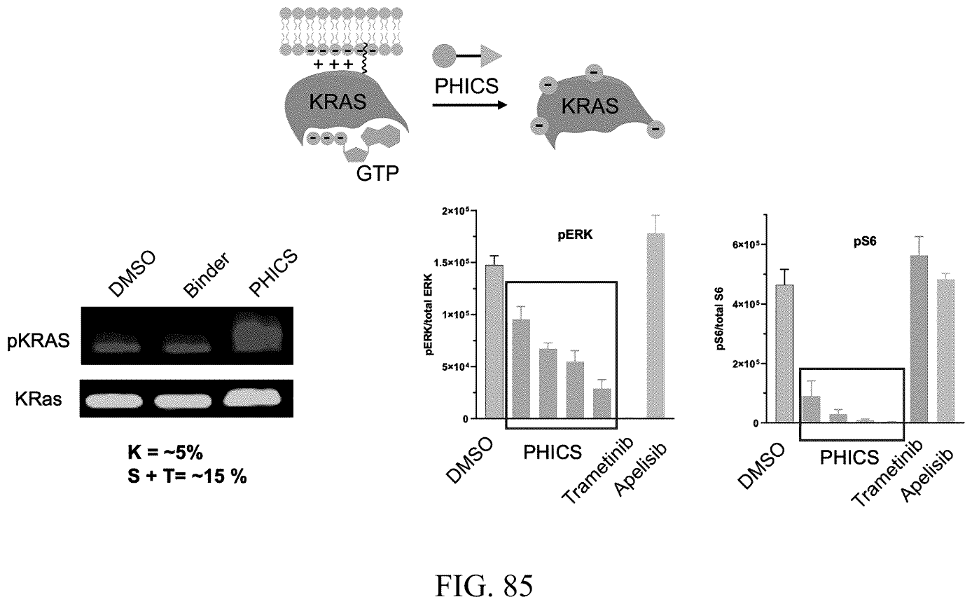

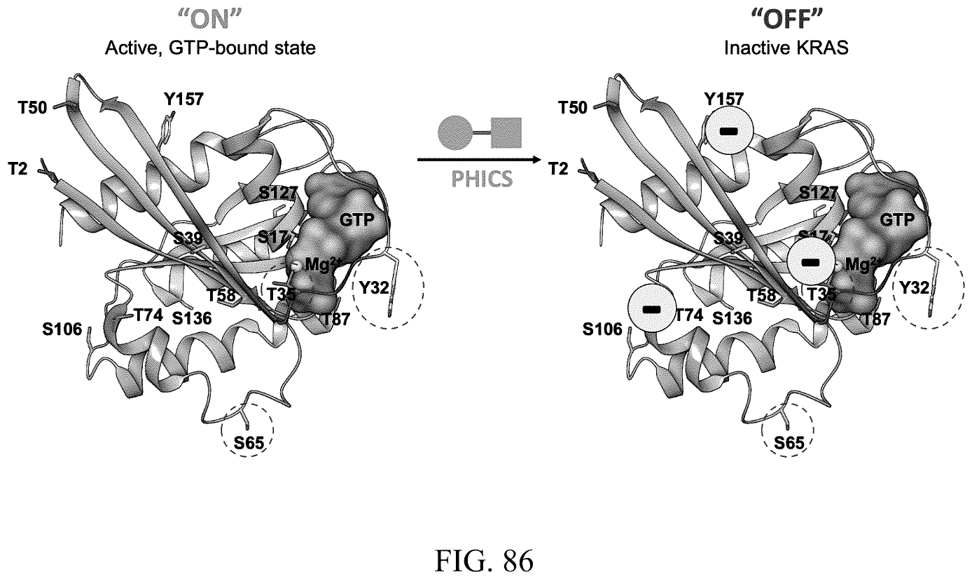

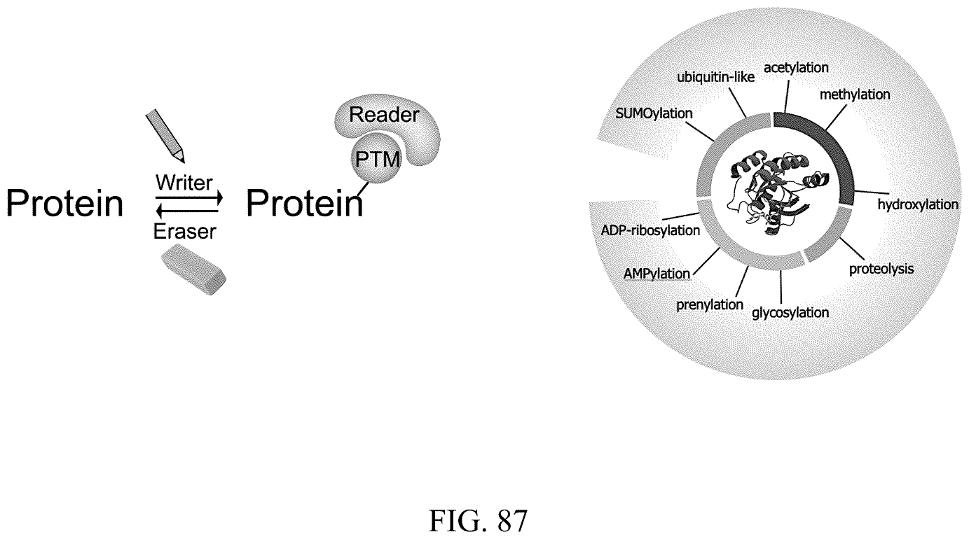

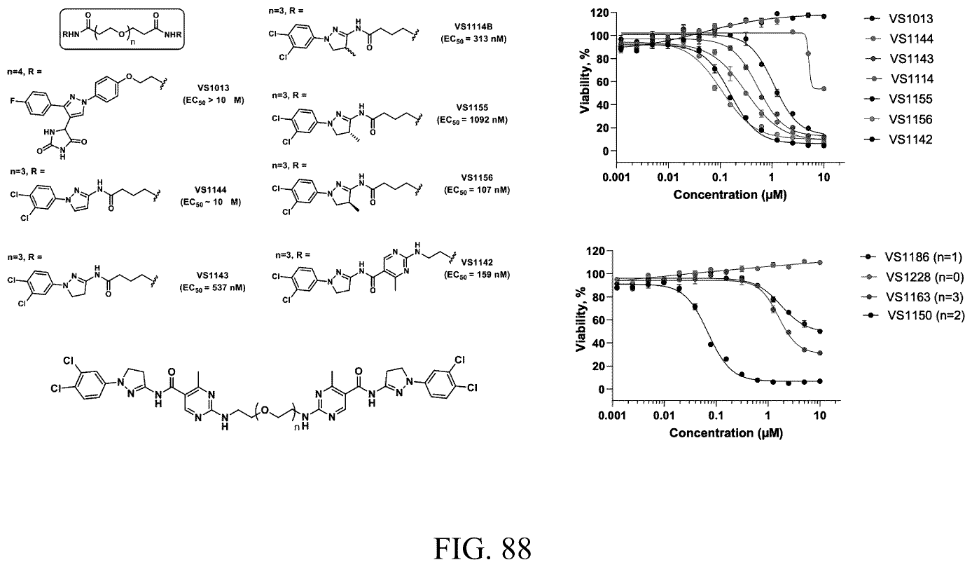

In one example embodiment the attachment to the ABL kinase activator dihydropyrazol is via various types of linkers, see, e.g. (PHICS 10.1-10.5, FIG. 64A of PCT/US2021/012816).

In one preferred embodiment, one of the ABL kinase binding moieties as detailed herein is used with a BRD4 binding moiety as described herein in a chimeric small molecule. In one example embodiment, when the protein binding moiety is for BRD4, an exemplary molecule of

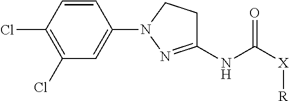

may comprise:

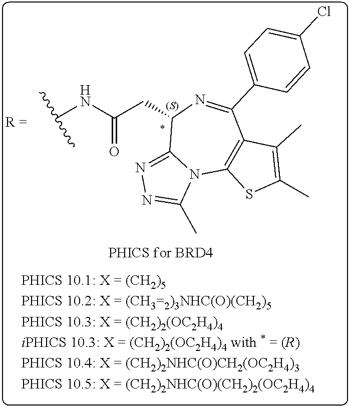

In one embodiment, the kinase binding moiety is an ABL kinase binding moiety according to formula

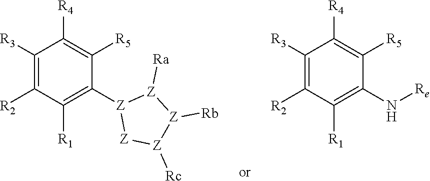

wherein, R1-R5 are independently selected from H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; or an aliphatic halide such as —OCF₂Cl; Z is independently selected from B, C, N, O, S, preferably wherein 1 or 2 atoms of Z═N, O, S, or a combination thereof; Ra, Rb, Rc, are independently selected from alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, aliphatic halide such as —OCF₂Cl; cyclic hydrocarbon, an unsaturated cyclic hydrocarbon, a heterocycle; one or more fused rings comprising any combination of any previously mentioned rings thereof; and Re is alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, aliphatic halide such as —OCF₂Cl; cyclic hydrocarbon, an unsaturated cyclic hydrocarbon, a heterocycle; one or more fused rings thereof at one or more positions, or can form a ring together with R1 or R5, or any combination thereof.

In one embodiment, the one or more of Rₐ, R_{b}, R_{c} is an amide further bonded to a molecule selected from the group consisting of;

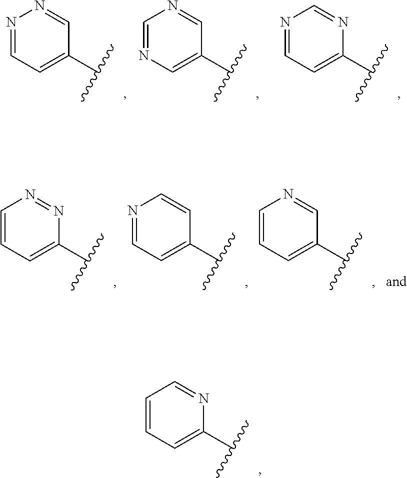

which can be optionally further substituted with alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, cyclic hydrocarbon, an unsaturated cyclic hydrocarbon, a heterocycle; or any combination thereof group at one or more positions.

In one embodiment, the ABL binding moiety is according to formula II(b), wherein Re is selected from the group consisting of

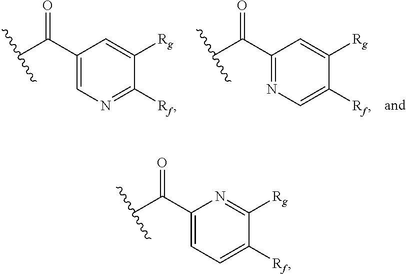

wherein Rf and Rg are selected from cyclic hydrocarbon; an unsaturated cyclic hydrocarbon; a heterocycle; one or more fused rings comprising any combination of any previously mentioned rings optionally substituted at one or more positions alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, cyclic hydrocarbon, an unsaturated cyclic hydrocarbon, a heterocycle; one or more fused rings comprising any combination of any previously mentioned rings. In one example embodiment, wherein Rf and Rg are independently selected from the group consisting of,

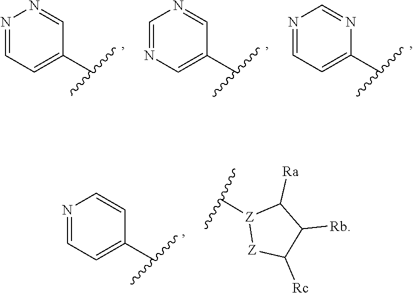

In one embodiment, the kinase binding moiety is selected from the group consisting of;

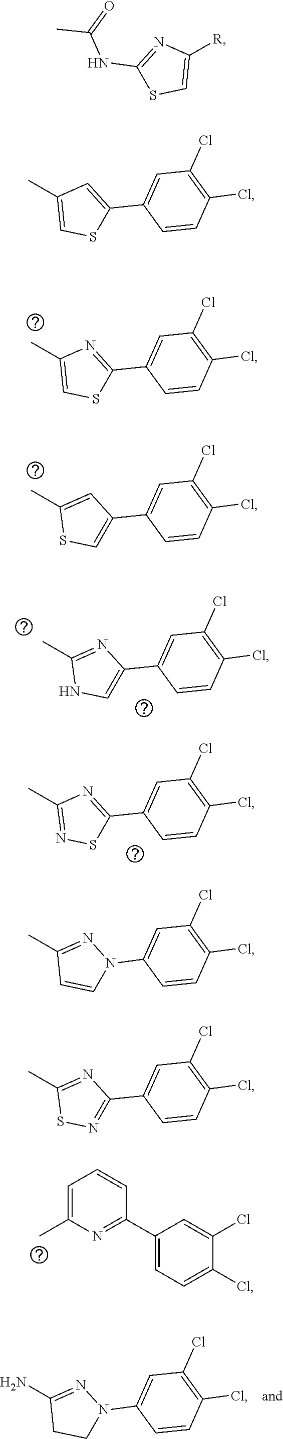

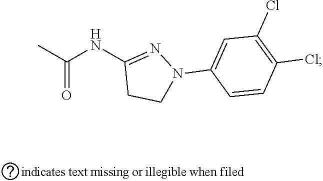 [SMILES: CC(=O)NC1=NN(c2ccc(Cl)c(Cl)c2)CC1]

wherein R selected from H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; an aliphatic halides such as —OCF₂Cl or any combination thereof; and optionally selected from

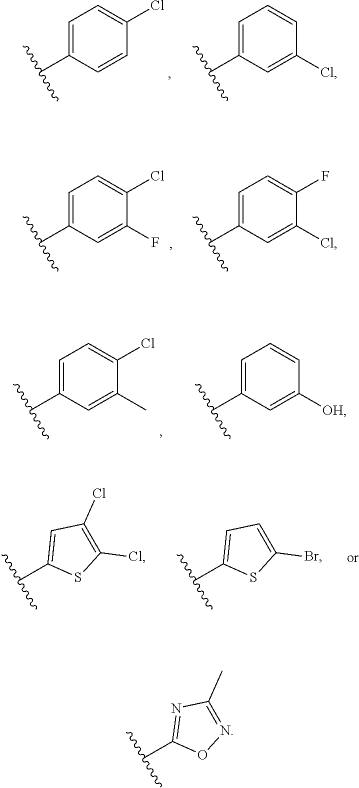

In one example embodiment, the kinase binding moiety is a ABL kinase inhibitor. In one example embodiment, the ABL inhibitor is Imatinib with the formula:

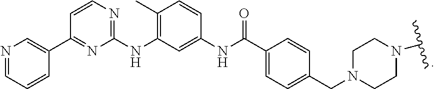 [SMILES: *N1CCN(Cc2ccc(C(=O)Nc3ccc(C)c(Nc4nccc(-c5cccnc5)n4)c3)cc2)CC1]

In a one example embodiment, the ABL inhibitor is Nilotinib, Dasatinib, Bosutinib, Ponatinib, or any derivative thereof. In other preferred example embodiments, the kinase binding molecules selected from:

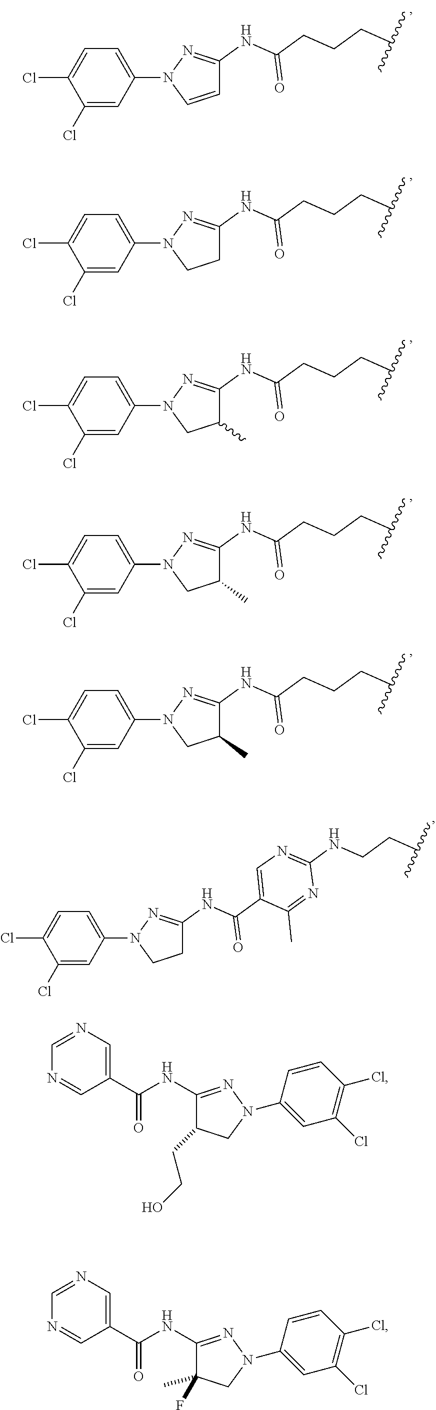

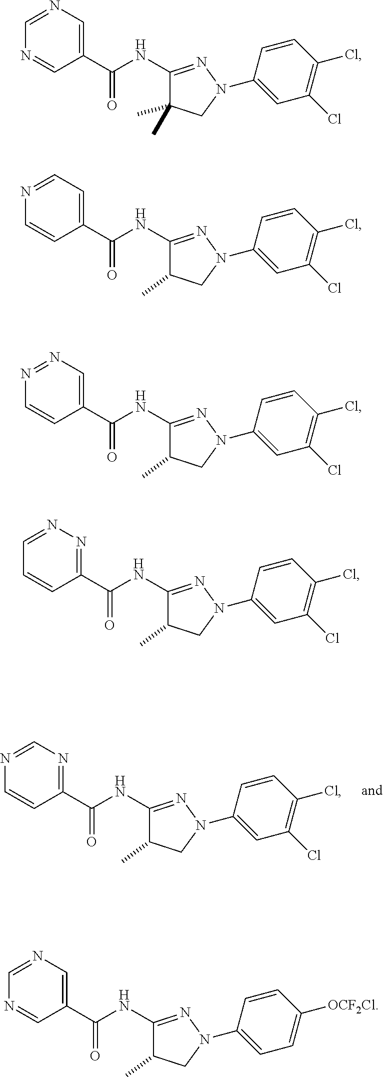

In one embodiment, the ABL kinase binding molecule is selected from the group consisting of

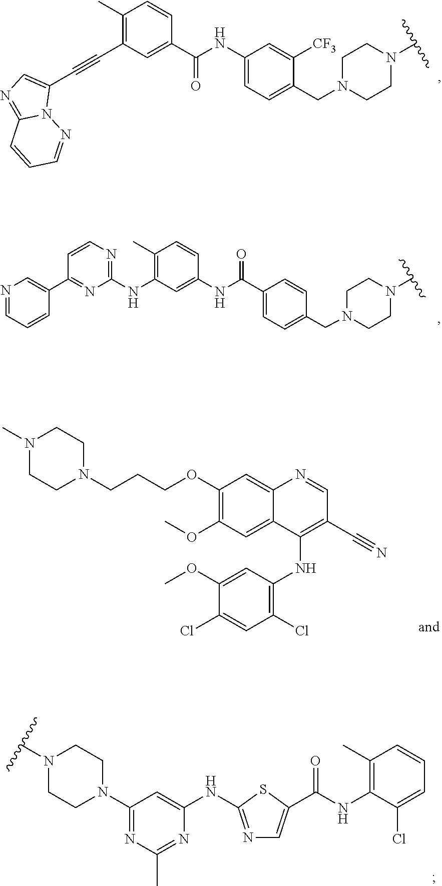 [SMILES: *N1CCN(Cc2ccc(NC(=O)c3ccc(C)c(C#Cc4cnc5cccnn45)c3)cc2C(F)(F)F)CC1 ; *N1CCN(Cc2ccc(C(=O)Nc3ccc(C)c(Nc4nccc(-c5cccnc5)n4)c3)cc2)CC1 ; COc1cc(Nc2c(C#N)cnc3cc(OCCCN4CCN(C)CC4)c(OC)cc23)c(Cl)cc1Cl ; *N1CCN(c2cc(Nc3ncc(C(=O)Nc4c(C)cccc4Cl)s3)nc(C)n2)CC1]

In one embodiment, the ABL kinase binding molecule is selected from the group consisting of;

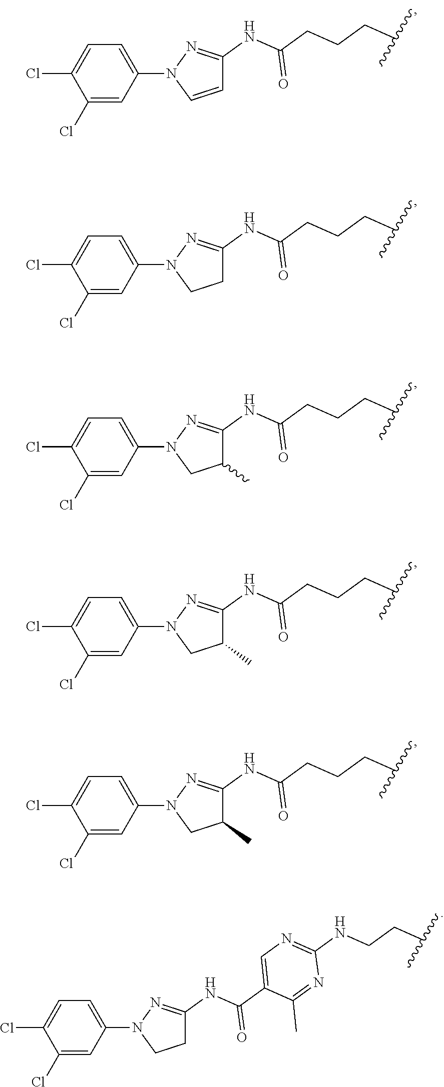 [SMILES: *CCCC(=O)Nc1ccn(-c2ccc(Cl)c(Cl)c2)n1 ; *CCCC(=O)NC1=NN(c2ccc(Cl)c(Cl)c2)CC1 ; *CCCC(=O)NC1=NN(c2ccc(Cl)c(Cl)c2)CC1C ; *CCCC(=O)NC1=NN(c2ccc(Cl)c(Cl)c2)C[C@H]1C ; *CCCC(=O)NC1=NN(c2ccc(Cl)c(Cl)c2)C[C@@H]1C ; *CCNc1ncc(C(=O)NC2=NN(c3ccc(Cl)c(Cl)c3)CC2)c(C)n1]

In one embodiment, the ABL kinase binding molecule is selected from the group consisting of;

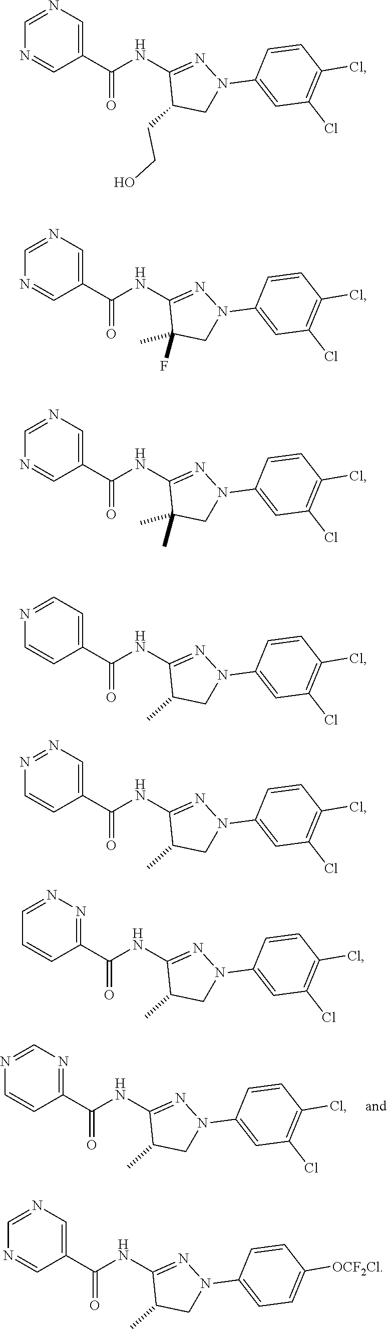

In an example embodiment, the ABL kinase binding moiety is Asciminib, also known as ABL-001, according to the formula:

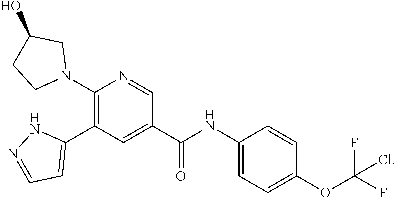 [SMILES: O=C(Nc1ccc(OC(F)(F)Cl)cc1)c1cnc(N2CC[C@@H](O)C2)c(-c2ccn[nH]2)c1]

| Hydrogen bond acceptors | [symbol] |
|---|---|
| Hydrogen bond donors | 3 |
| Rotatable bonds | 7 |
| Topological polar surface area | 103.37 |
| Molecular weight | 449.11 |
| XLogP | 4.3 |
| No. Lipinski's rules broken | 0 |

[symbol]

indicates data missing or illegible when filed

Asciminib is a negative allosteric modulator of BCR-ABL1, that induces the kinase to adopt an autoinhibitory, and thereby inactive, conformation. Asciminib-based PROTACs have been given the Fast-Track designation. In the UK, asciminib is available for compassionate use, on a named Organ function impairment and was shown to have minimal effect on platelet function. Asciminib has inhibitory action on cellular proliferation in vitro with GI₅₀ 1.5 nM for the wild type ABL1 cell line and 35 nM for the ABL1T315I cell line. Asciminib also has an pIC₅₀ of 8.6-9.5 for ABL proto-oncogene 1, non-receptor tyrosine kinase. See e.g. Schoepfer, J., et al. “Discovery of Asciminib (ABL001), an Allosteric Inhibitor of the Tyrosine Kinase Activity of BCR-ABL1.” J. Med. Chem. 2018, 61 (18), 8120-8135, herein incorporated by reference in its entirety. In an aspect, the compound is according to:

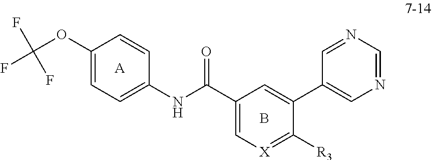

| | | | | | | | | | | Rat liver | |
|---|---|---|---|---|---|---|---|---|---|---|---|
| | | | | | | | | | | [symbol] | hERG |
| | | | | | | | | | HT- | [symbol] CL | def. |
| | | | | Luc-B [symbol] | Luc-B [symbol] | | | | perm | (ml · | Binding |
| | | | ABL1 [symbol] | BCR-ABL1 [symbol] | BCR-ABL1 [symbol] | clogP/ | | FASSIF | calc PA | min [symbol] | IC₅₀ |
| Cpd | R² | X | IC₅₀ (μM) | GI₅₀ (μM) | GI₅₀ (μM) | logP | pKa | (mM) | (%) | kg [symbol] ) | (μM) |
| 6 | | | 0.55 ± [symbol] | 0.253; 0.341 | 2.93; >10 | 3.6/4.2 | 6.2 | 0.499 | 100 | 147 | 3.7 |
| 7 | 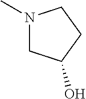 | N | 0.0023 ± [symbol] | 0.0017 ± [symbol] | 0.073 ± [symbol] | 2.0/3.0 | 3.3 | 0.59 | 56 | 40 | 9.6 |
| 8 | H | CH | 0.018 ± [symbol] | 0.117 ± [symbol] | 1.80 ± 0.44 | 3.5/4.3 | 3.3 | 0.01 | 100 | 28 | >10 |
| 9 | H | N | 0.024 ± [symbol] | 0.078 ± [symbol] | 1.65 ± 0.44 | 2.5/2.7 | n/a | 0.026 | 98 | 20 | >30 |
| 10 | OMe | CH | 0.011 ± [symbol] | 0.004 ± [symbol] | 0.80 ± 0.09 | 2.9/4.6 | n/a | 0.16 | 100 | 16 | 1.1 |
| 11 | 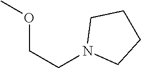 | CH | 0.019 ± [symbol] | 0.020 ± [symbol] | 1.82; 1.39 | 3.7/>3.4 | 8.9 | 0.3 | 99 | 60 | 0.009 |
| 12 | NH(CH₂) [symbol] OH | N | 0.018 ± [symbol] | 0.004; [symbol] | 0.511; 0.543 | 2.7/3.2 | 3.7 | 0.013 | 36 | 60 | 1.5 |
| 13 | 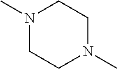 | N | 0.007 ± [symbol] | 0.005 ± [symbol] | 0.396 ± [symbol] | 3.1/n/a | 7.4 | 0.151 | 98 | 34 | 0.31 |
| 14 | 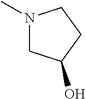 | N | 0.004 ± [symbol] | 0.004 ± [symbol] | 0.294 ± [symbol] | 2.0/3.5 | 3.7 | >1 | 40 | 50 | 12, |

[symbol]

indicates data missing or illegible when filed

from Schoepfer et al. (2018).

In an example embodiment, the ABL kinase binding moiety is BO1 according to the formula:

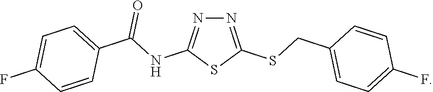 [SMILES: O=C(Nc1nnc(SCc2ccc(F)cc2)s1)c1ccc(F)cc1]

BO1 is a non-ATP competitive, negative allosteric modulator of mutant BCR-ABL kinase proteins. Interaction of BO1 with the wild type protein shows an ATP-competitive/mixed mechanism of action. BO1 has a pKᵢ of 7.0-7.4 for ABL proto-oncogene 1, non-receptor tyrosine kinases. See e.g. Radi, M., et al. “Discovery and SAR of 1,3,4-Thiadiazole Derivatives as Potent Abl Tyrosine Kinase Inhibitors and Cytodifferentiating Agents.” Bioorganic & Medicinal Chemistry Letters 2008, 18 (3), 1207-1211, herein incorporated by reference in its entirety., in particular, compounds 6a-6u:

TABLE 1
c-Src/Ab [symbol] tyrosine kinase inhibitory
activities exerted by compounds 6a-u

Activity^{a} (K [symbol] μM)

| Compound | R¹ | R² | c-Src | Ab [symbol] |
|---|---|---|---|---|
| 6a | p-F | p-F | 0.354 | 0.044 |
| 6b | p-Br | p-Cl | 0.217 | 0.047 |
| 6c | H | p-F | 0.464 | 0.070 |
| 6d | m-Cl | p-Cl | 0.195 | 0.073 |
| 6e | p-NO₂ | [symbol] -Cl | 0.219 | 0.083 |
| 6f | p-Br | p-F | 0.221 | 0.089 |
| 6g | p-NO₂ | p-Cl | 0.165 | 0.092 |
| 6h | p-F | p-Cl | 0.200 | 0.104 |
| 6i | m-F | p-F | 0.569 | 0.167 |
| 6j | p-OCH [symbol] | p-Cl | 0.199 | 0.189 |
| 6k | p-OCH [symbol] | p-F | 0.26 [symbol] | 0.1 [symbol] 5 |
| 6l | p-NO₂ | p-F | 0.170 | 0.210 |
| 6m | m-F | p-Cl | 0.064 | 0.217 |
| 6n | p-Br | [symbol] -Cl | 0.522 | 0.225 |
| 6o | m-F | [symbol] -Cl | 0.718 | 0.272 |
| 6p | m-Cl | p-F | 0.247 | 0.369 |
| 6q | H | p-Cl | 0.334 | 0.400 |
| 6r | p-F | [symbol] -Cl | 0.900 | 0.406 |
| 6s | m-Cl | [symbol] -Cl | 0.169 | 0.760 |
| 6t | H | [symbol] -Cl | 1.137 | 0.920 |
| 6u | p-OCH [symbol] | [symbol] -Cl | 0.272 | 1.260 |
| Imatinib | | | 31 [symbol] | 0.013 |

[symbol]

indicates data missing or illegible when filed

In one example embodiment, the ABL kinase binding moiety is GNF-2 according to the formula:

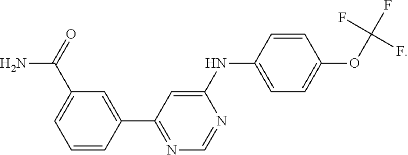 [SMILES: NC(=O)c1cccc(-c2cc(Nc3ccc(OC(F)(F)F)cc3)ncn2)c1]

GNF-2 is a highly selective non-ATP competitive inhibitor of Bcr-Abl. It acts as a negative allosteric modulator, binding to a site distant from the ATP pocket. GNF-2 inhibits the Bcr/Abl fusion protein with an IC₅₀ value of 267 nM. See e.g. Zhang, J., et al. “Targeting Bcr-Abl by Combining Allosteric with ATP-Binding-Site Inhibitors.” Nature 2010, 463 (7280), 501-506, herein incorporated by reference in its entirety.

In one example embodiment, the ABL kinase binding moiety is GNF-5 according to the formula:

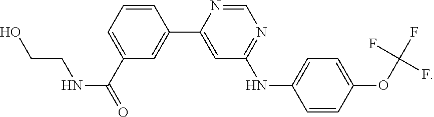 [SMILES: O=C(NCCO)c1cccc(-c2cc(Nc3ccc(OC(F)(F)F)cc3)ncn2)c1]

GNF-5 is a selective and allosteric BCR-ABL inhibitor. GNF-5 can largely overcome the resistance patterns associated with imatinib or nilotinib treatment (except for the gatekeeper mutation T315I). Co-treatment with GNF-2 (GNF-5's original structural incarnation) plus imatinib significantly decreases the emergence of resistant clones in vitro. GNF-5 downregulates BCR-ABL kinase activity by mimicking the effect of myristate binding, which directs the protein towards adopting an inactive conformational state. GNF-5 has pIC₅₀ of 6.7 for ABL proto-oncogene 1, non-receptor tyrosine kinase. See e.g. Deng, X., et al. “Expanding the Diversity of Allosteric Bcr-Abl Inhibitors.” J. Med. Chem. 2010, 53 (19), 6934-6946, herein incorporated by reference in its entirety, and Zhang Nature 2010. In an aspect, SAR can be performed around the GNF-2 scaffold, with functionality modified at particular positions:

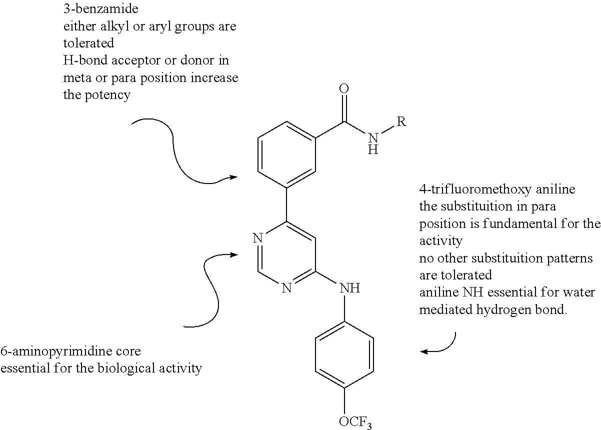

The crystal structure of GNF-2 bound to the Abl myristoyl pocket can also be utilized for further optimization., see, FIG. 2 of Zhang, Nature, 2010 463, 501-506, incorporated herein by reference. Co-crystal structure of imatibinib and GNF-2 in complex with c-Abl is also available (PDB ID: 3K5V). Additional targeting moieties can be designed as described in FIG. 3 of Zhang Nature (2010: FIG. 3, incorporated by reference and depicted below:

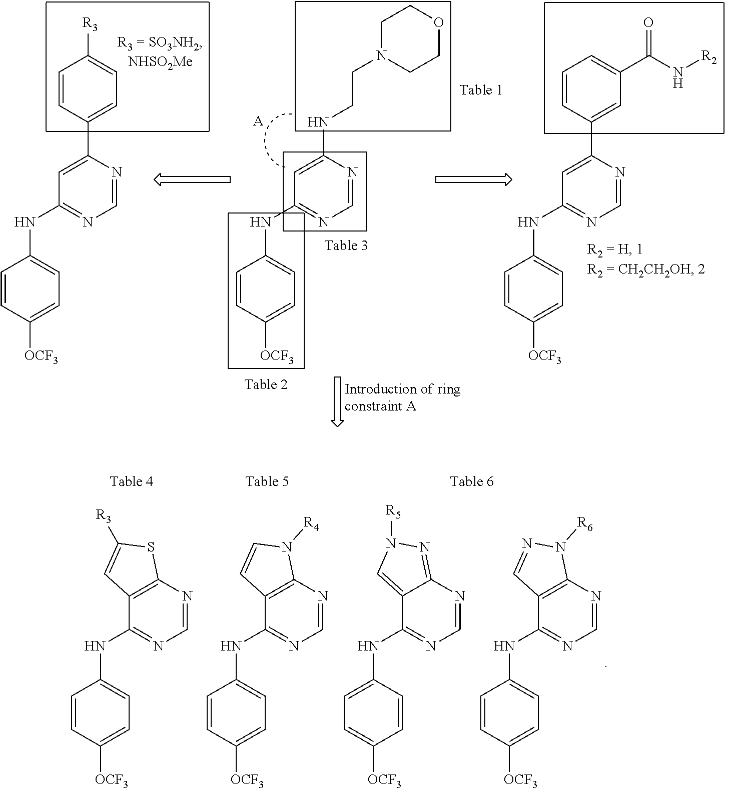

In an example embodiment, the ABL kinase binding moiety is DPH according to the formula:

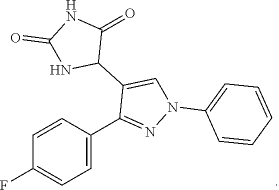 [SMILES: O=C1NC(=O)C(c2cn(-c3ccccc3)nc2-c2ccc(F)cc2)N1]

DPH has an ICW EC₅₀ of 6.1, see e.g. Simpson, G. L., et al. “Identification and Optimization of Novel Small C-Abl Kinase Activators Using Fragment and HTS Methodologies.” J. Med. Chem. 2019, 62 (4), 2154-2171, herein incorporated by reference in its entirety, and may be according to DPH and compounds as identified below:

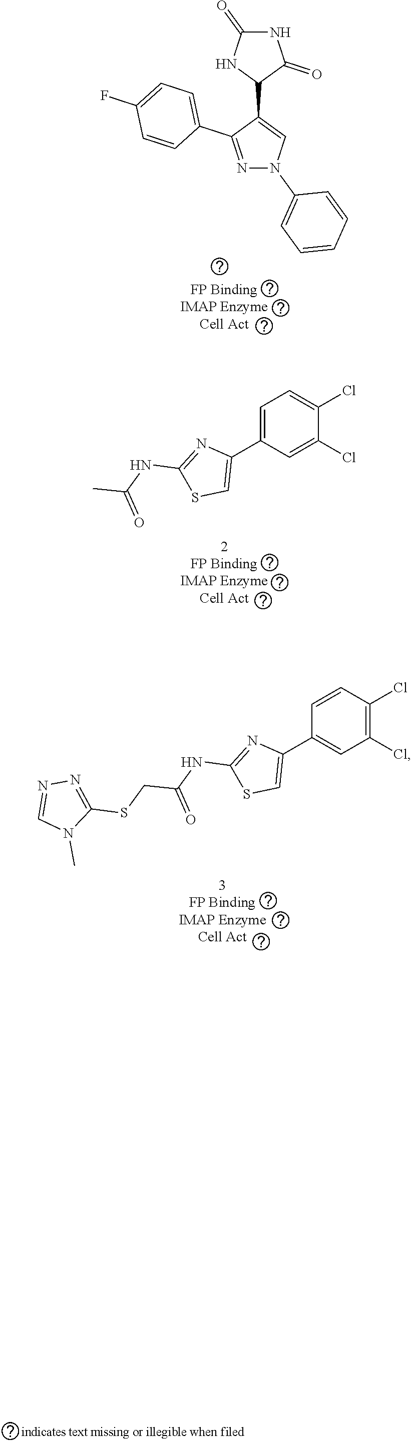

as well as compounds 45 and 32 as adapted from Simpson et al.:

| | | | In Vitro | | | | |
|---|---|---|---|---|---|---|---|
| | | cellular | Clear- | | | Fold Increase | |
| | | acti- | ance [symbol] | In Vivo Plasma | | pkCRKL Vs | |
| | | vation | (mL/min/ | Concentration | | Total Crk-L | |
| | | (ICW | mg | (mg/mL) [symbol] | | (normalised) [symbol] | |
| Cmpd | Structure | EC₅₀) | protein) | 48 min | 180 min | 40 min | 180 min |
| 1 | 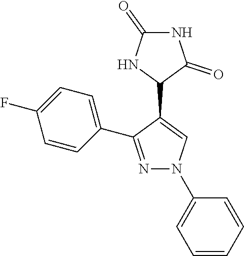 | 6.1 | 0.266 | 15100 ± 1350 | 1050 ± 595 | 13x | 28x |
| 45 | 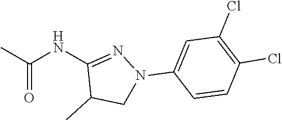 [SMILES: CC(=O)NC1=NN(c2ccc(Cl)c(Cl)c2)CC1C] | 6.3 | 0.408 | 5940 ± 233 | 2640 ± 343 | 1x | 5x |
| 32 | 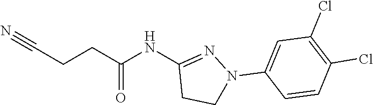 [SMILES: N#CCCC(=O)NC1=NN(c2ccc(Cl)c(Cl)c2)CC1] | 6.1 | <0.01 | 15600 ± 1700 | 12600 ± 3530 | 19x | 35x. |

[symbol]

indicates data missing or illegible when filed

In another example embodiment, the ABL target binding moiety is any c-ABL kinase activator from Simpson J. Med. Chem. 2019.

In an example embodiment, the ABL kinase binding moiety is dihydropyrazole according the formula:

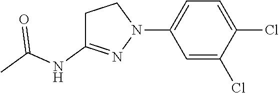 [SMILES: CC(=O)NC1=NN(c2ccc(Cl)c(Cl)c2)CC1]

In one example embodiments, the ABL kinase binding moiety is selected from the group consisting of:

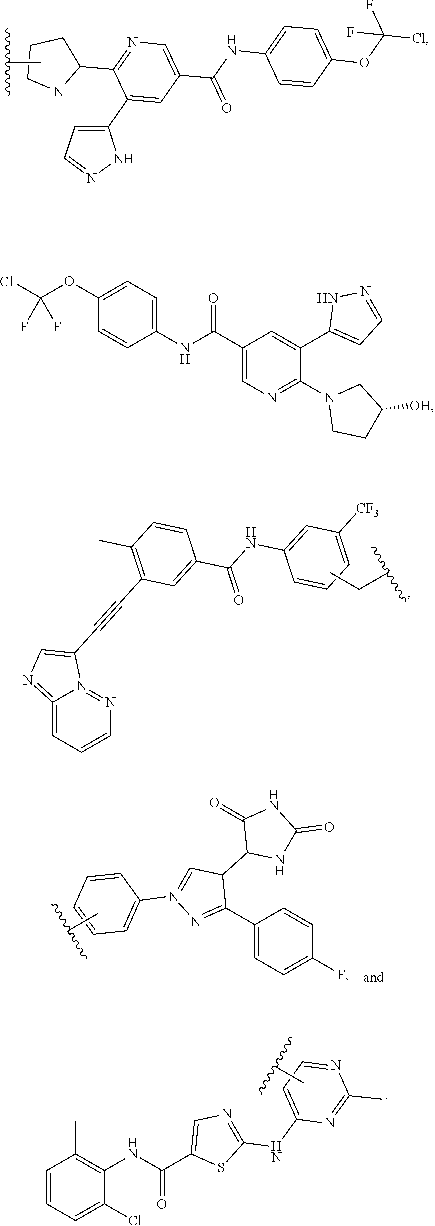

In one embodiment, the ABL kinase binding moiety

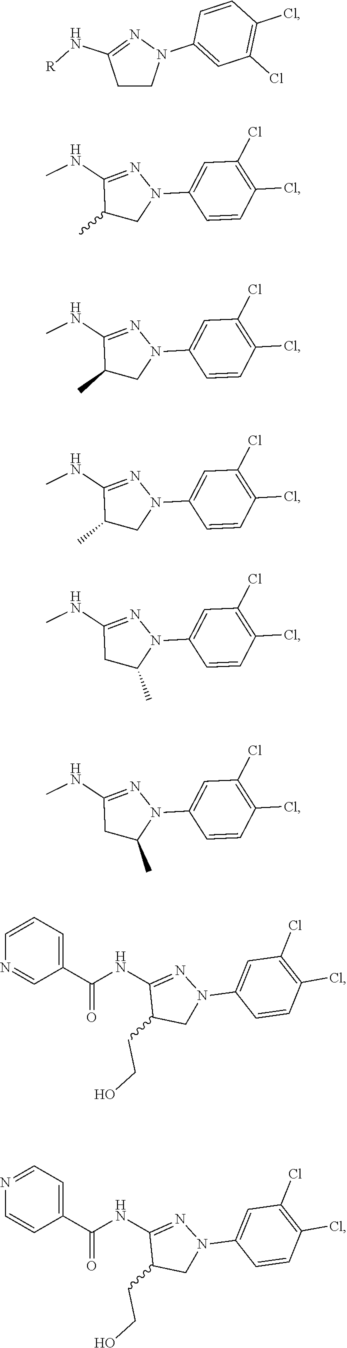

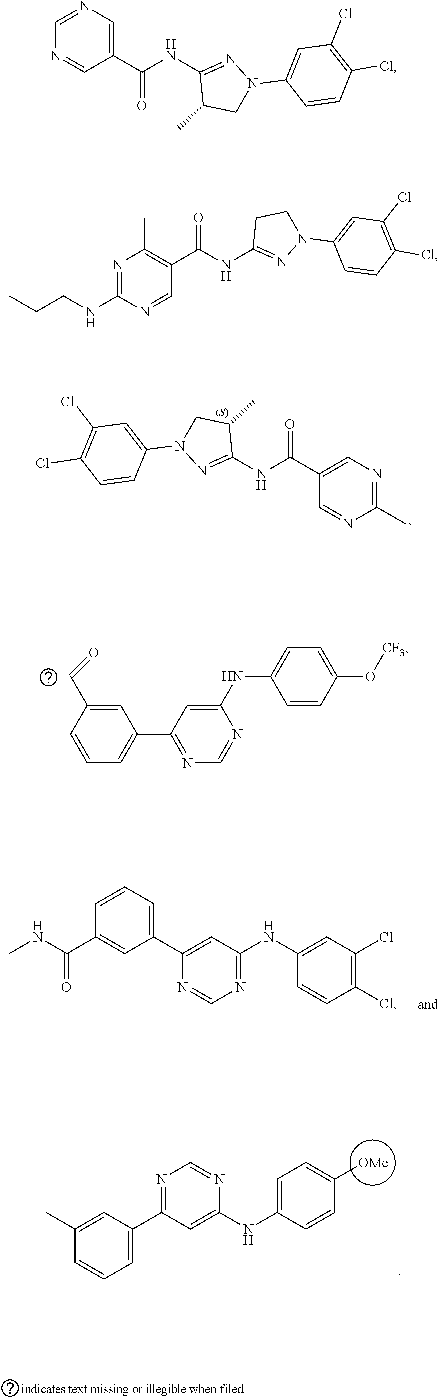

The R in ABL inhibitor formula is optimized for physiochemical properties, such as solubility and/or permeability, and/or pharmacokinetic properties, such as microsomal stability or target binding. In one example embodiment, R is selected from any boron-, carbon-, nitrogen-, oxygen-, sulfur-, halogen-based substituent, heterocycle, fused ring, or any combination thereof. In preferred example embodiments, R is selected from H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; an aliphatic halides such as —OCF₂Cl or any combination thereof. In preferred example embodiments, R is selected from

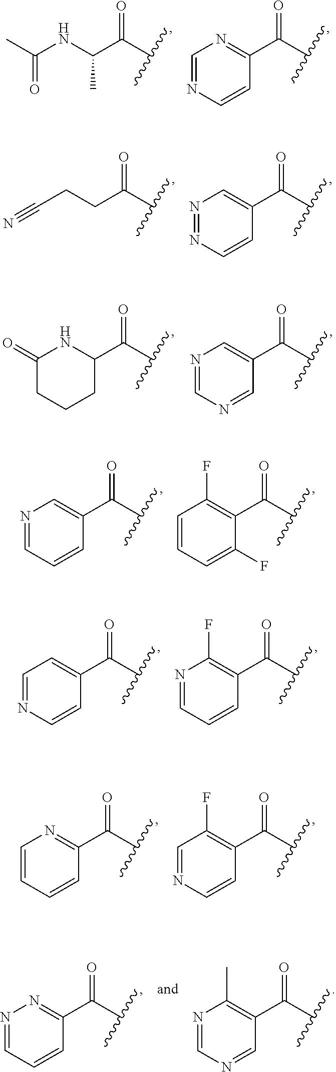

In an example embodiment, the ABL inhibitor kinase binding molecule is selected from

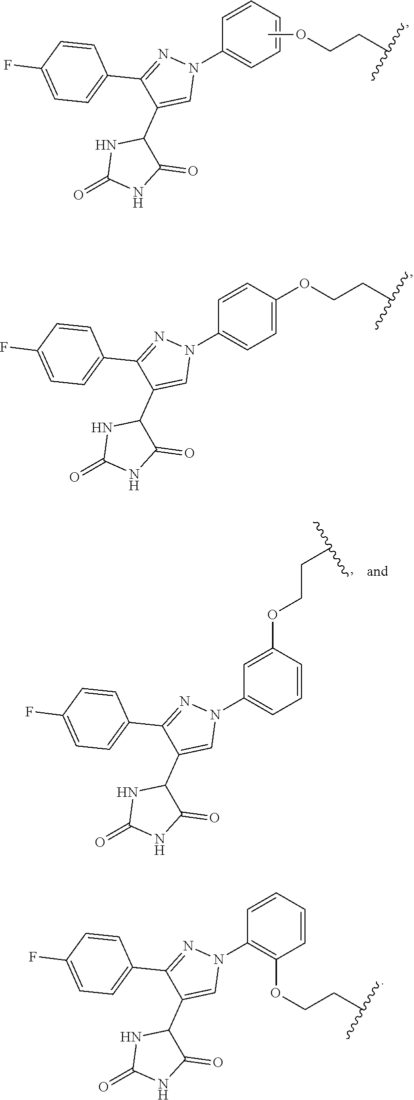

In other example embodiments, the ABL inhibitor kinase binding molecule is selected from the formula

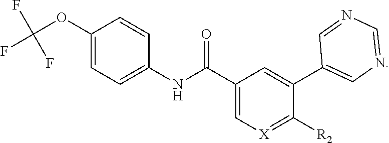

In one example embodiment, X and R₂ is optimized for physiochemical properties, such as solubility and/or permeability, and/or pharmacokinetic properties, such as microsomal stability or target binding. In one example embodiment, X is any feasible boron-, carbon-, nitrogen-, oxygen-, or sulfur-based element or compound. In preferred example embodiments, X is selected from C, N, O, and S. In one example embodiment, R₂ is selected from any boron-, carbon-, nitrogen-, oxygen-, sulfur-, halogen-based substituent, heterocycle, fused ring, or any combination thereof. In preferred example embodiments, R₂ is selected from R2 is selected from H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; an aliphatic halides such as —OCF₂Cl or any combination thereof. In Preferred example embodiments, R₂ is selected from

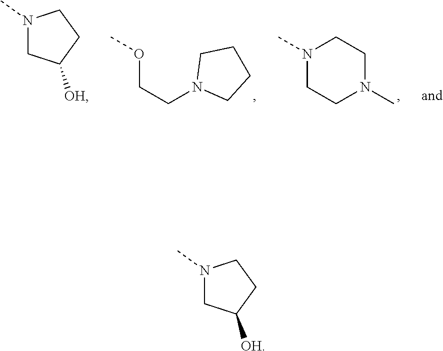

In one example embodiments, the ABL kinase binding molecule is selected from

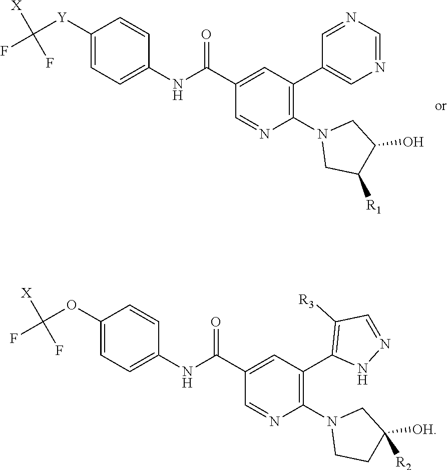

In one example embodiment, X, Y, and R groups are optimized for physiochemical properties, such as solubility and/or permeability, and/or pharmacokinetic properties, such as microsomal stability or target binding. In one example embodiment, X and Y are independently selected from any boron-, carbon-, nitrogen-, oxygen-, sulfur-, halogen-based substituent, heterocycle, fused ring, or any combination thereof. In preferred example embodiments, X is a halogen. In preferred example embodiments, Y is selected from C, N, O, and S. In one example embodiment, R₁, R₂, and R₃ is independently selected from any boron-, carbon-, nitrogen-, oxygen-, sulfur-, halogen-based substituent, heterocycle, fused ring, or any combination thereof. In preferred example embodiments, R₁, R₂, and R₃ is independently selected from H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; an aliphatic halides such as —OCF₂Cl or any combination thereof.

In other example embodiments, the ABL inhibitor kinase binding molecule is selected from:

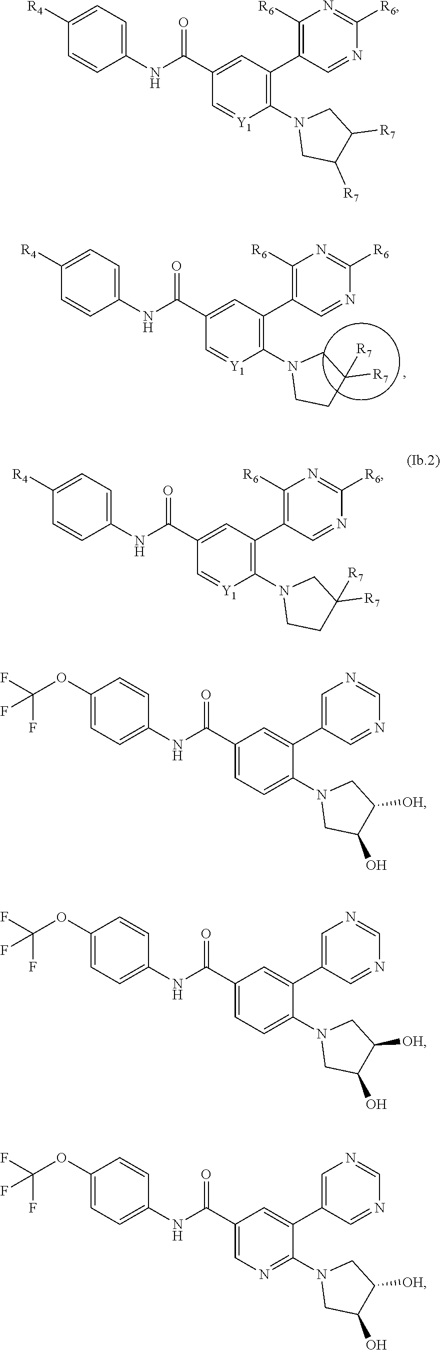

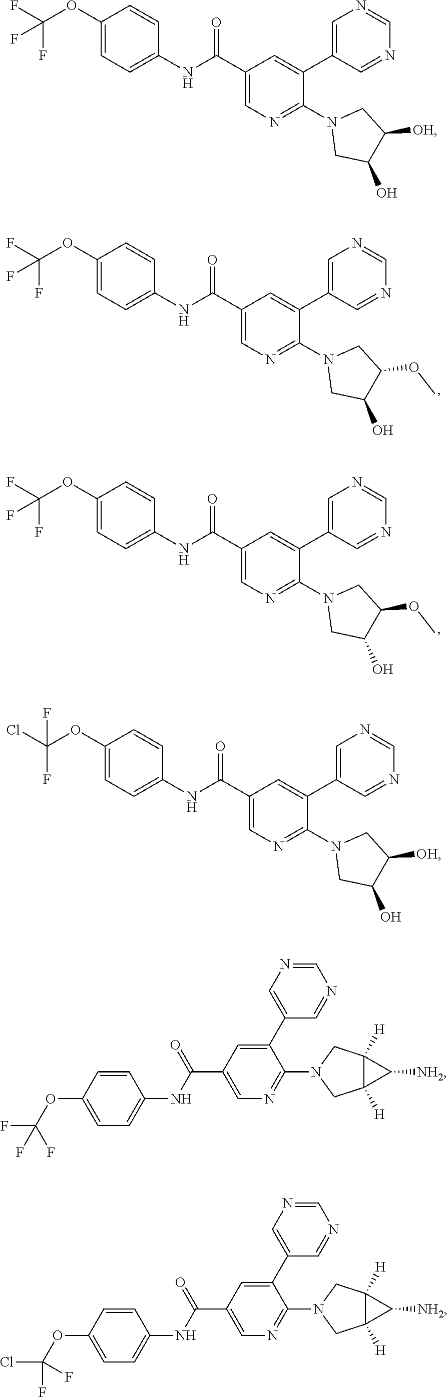 [SMILES: O=C(Nc1ccc(OC(F)(F)F)cc1)c1cnc(N2C[C@@H](O)[C@@H](O)C2)c(-c2cncnc2)c1 ; CO[C@H]1CN(c2ncc(C(=O)Nc3ccc(OC(F)(F)F)cc3)cc2-c2cncnc2)C[C@@H]1O ; CO[C@@H]1CN(c2ncc(C(=O)Nc3ccc(OC(F)(F)F)cc3)cc2-c2cncnc2)C[C@H]1O ; O=C(Nc1ccc(OC(F)(F)Cl)cc1)c1cnc(N2C[C@@H](O)[C@@H](O)C2)c(-c2cncnc2)c1 ; N[C@@H]1[C@H]2CN(c3ncc(C(=O)Nc4ccc(OC(F)(F)F)cc4)cc3-c3cncnc3)C[C@@H]12 ; N[C@@H]1[C@H]2CN(c3ncc(C(=O)Nc4ccc(OC(F)(F)Cl)cc4)cc3-c3cncnc3)C[C@@H]12]

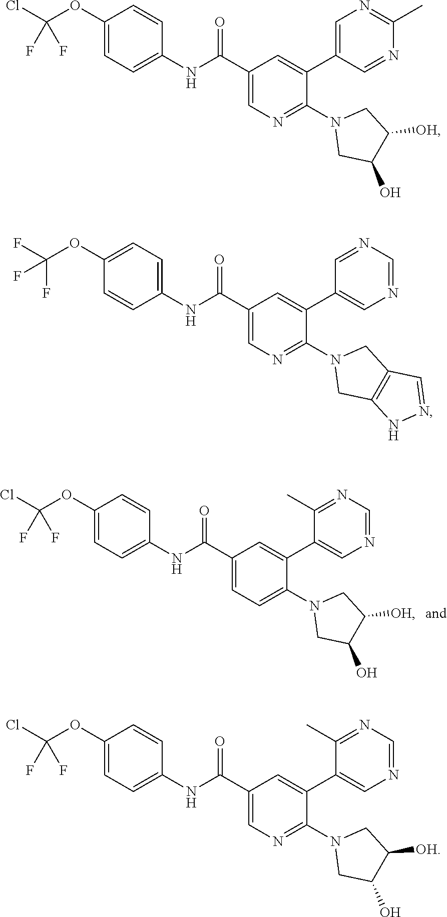 [SMILES: Cc1ncc(-c2cc(C(=O)Nc3ccc(OC(F)(F)Cl)cc3)cnc2N2C[C@H](O)[C@@H](O)C2)cn1 ; O=C(Nc1ccc(OC(F)(F)F)cc1)c1cnc(N2Cc3cn[nH]c3C2)c(-c2cncnc2)c1 ; Cc1ncncc1-c1cc(C(=O)Nc2ccc(OC(F)(F)Cl)cc2)ccc1N1C[C@H](O)[C@@H](O)C1 ; "and" ; Cc1ncncc1-c1cc(C(=O)Nc2ccc(OC(F)(F)Cl)cc2)cnc1N1C[C@@H](O)[C@H](O)C1]

In one example embodiment, the Y groups and R groups are optimized for physiochemical properties, such as solubility and/or permeability, and/or pharmacokinetic properties, such as microsomal stability or target binding. In one example embodiment, Y and Y₁ in the previously mentioned formulas is any feasible boron-, carbon-, nitrogen-, oxygen-, or sulfur-based element or compound. In preferred example embodiments, Y and Y₁ is selected from C, N, O, and S. In one example embodiment, R₃, R₄, R₆, and R₇ in the previously mentioned formulas are independently selected from any boron-, carbon-, nitrogen-, oxygen-, sulfur-, halogen-based substituent, heterocycle, fused ring, or any combination thereof. In Preferred example embodiments, R₃, R₄, R₆, and R₇ is independently selected from H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; an aliphatic halides such as —OCF₂Cl or any combination thereof.

In other example embodiments, the ABL kinase inhibitor binding molecule is selected from:

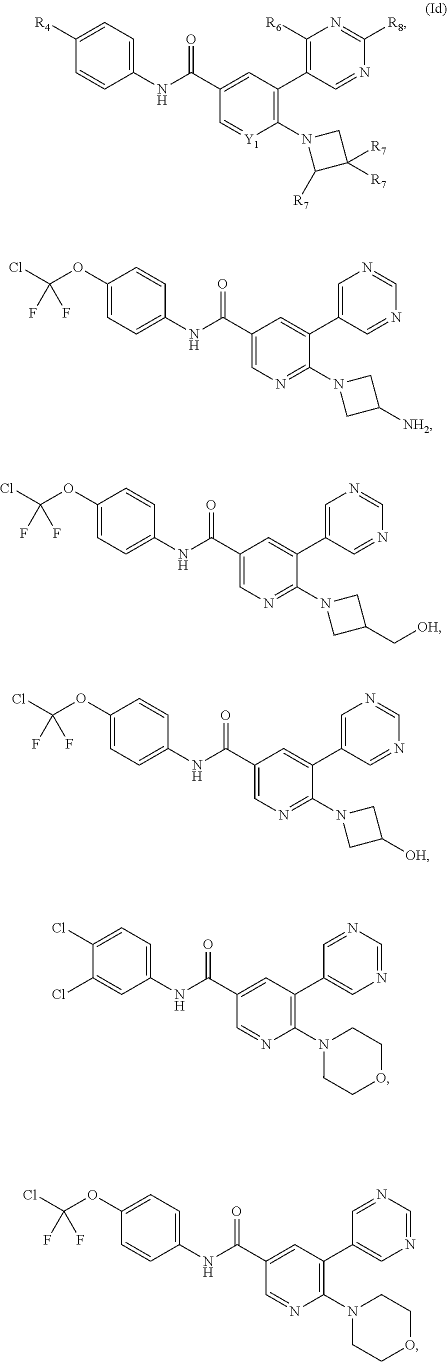

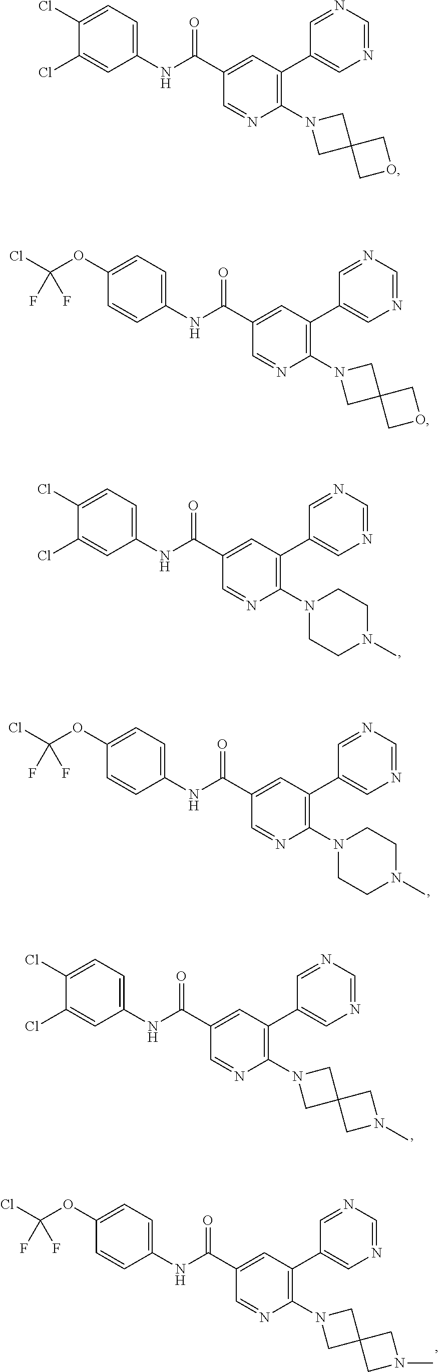 [SMILES: O=C(Nc1ccc(Cl)c(Cl)c1)c1cnc(N2CC3(COC3)C2)c(-c2cncnc2)c1 ; O=C(Nc1ccc(OC(F)(F)Cl)cc1)c1cnc(N2CC3(COC3)C2)c(-c2cncnc2)c1 ; CN1CCN(c2ncc(C(=O)Nc3ccc(Cl)c(Cl)c3)cc2-c2cncnc2)CC1 ; CN1CCN(c2ncc(C(=O)Nc3ccc(OC(F)(F)Cl)cc3)cc2-c2cncnc2)CC1 ; CN1CC2(C1)CN(c1ncc(C(=O)Nc3ccc(Cl)c(Cl)c3)cc1-c1cncnc1)C2 ; CN1CC2(C1)CN(c1ncc(C(=O)Nc3ccc(OC(F)(F)Cl)cc3)cc1-c1cncnc1)C2]

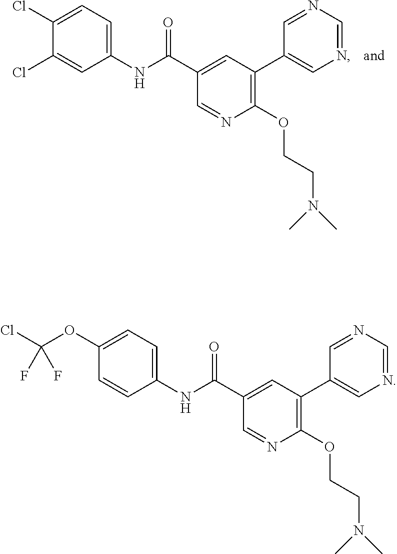 [SMILES: CN(C)CCOc1ncc(C(=O)Nc2ccc(Cl)c(Cl)c2)cc1-c1cncnc1 ; CN(C)CCOc1ncc(C(=O)Nc2ccc(OC(F)(F)Cl)cc2)cc1-c1cncnc1]

In one example embodiment, Y₁ and R groups are optimized for physiochemical properties, such as solubility and/or permeability, and/or pharmacokinetic properties, such as microsomal stability or target binding. In one example embodiment, Y₁ in the previously mentioned formulas is any feasible boron-, carbon-, nitrogen-, oxygen-, or sulfur-based element or compound. In Preferred example embodiments, Y₁ is selected from C, N, O, and S. In One example embodiment, R₄, R₆, and R₇ in the previously mentioned formulas are independently selected from any boron-, carbon-, nitrogen-, oxygen-, sulfur-, halogen-based substituent, heterocycle, fused ring, or any combination thereof. In Preferred example embodiments, R₄, R₆, and R₇ is independently selected from H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; an aliphatic halides such as —OCF₂Cl or any combination thereof.

In one example embodiment, the protein binding moiety is an ABL inhibitor. In one example embodiment, the ABL inhibitor is DCC-2036, which is a dual-anchoring inhibitor that binds both the switch control pocket E282/R386 pair and the Met318 ATP hinge with an IC₅₀ value of 0.8 nM according to the formula:

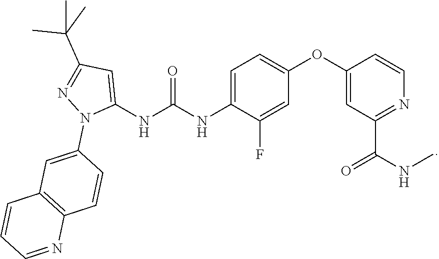 [SMILES: CNC(=O)c1cc(Oc2ccc(NC(=O)Nc3cc(C(C)(C)C)nn3-c3ccc4ncccc4c3)c(F)c2)ccn1]

In one example embodiment, the kinase binding moiety is a c-ABL tyrosine kinase inhibitor or any derivative thereof from the International Patent Application WO2019173761, herein incorporated by reference.

### AMPK Binding Moiety

In one example embodiment, the kinase binding moiety is an AMPK kinase binding moiety. AMPK is a serine/threonine kinase that assembles into a heterotrimeric complex composed of a catalytic α-subunit and two regulatory β- and γ-subunits. See, e.g. Wells et al. (2012). It is believed that small molecules that mimic AMP binding to the γ-subunit could directly activate AMPK.

In one embodiment, the AMPK kinase binding moiety is according to the formula:

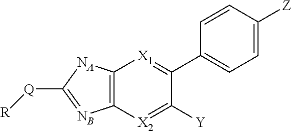

wherein R is selected from the group consisting of:

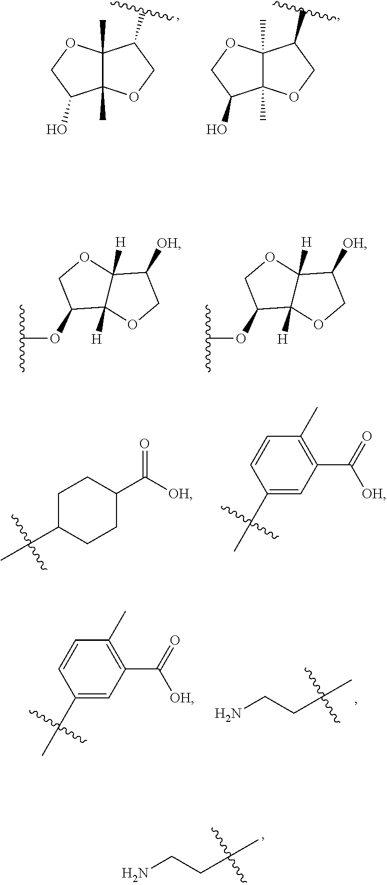

a carbohydrate mimetic, a heterocycle, a diahydrohexitol, a pyranose, or a furanose; Q is selected from the group consisting of: B, C, N, O, S; and wherein a H is located on either N_{A} or N_{B}; X₁ and X₂ is independently selected from the group consisting of: C, N and O; Y is selected from the group consisting of: H, OH, a halogen, CN or hydrogen bond donating substituent; and Z is selected from the group consisting of: H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; or an aliphatic halide such as —OCF₂Cl which optionally can be further substituted.

In one example embodiment, Z can be according to the formula:

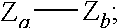

wherein Zₐ is selected from the group consisting of:

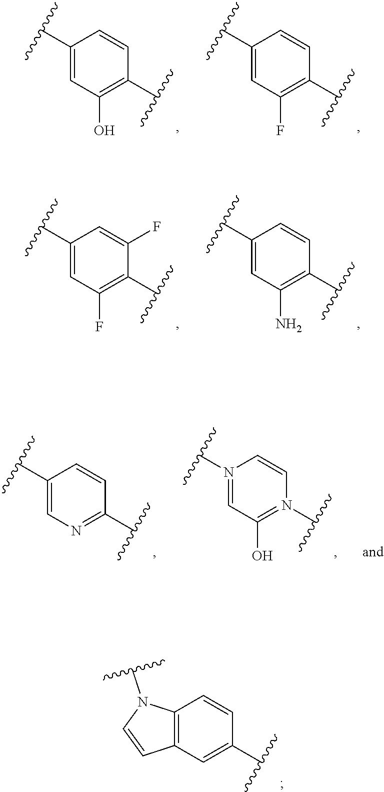

wherein Z_{b} is selected from the group consisting of:

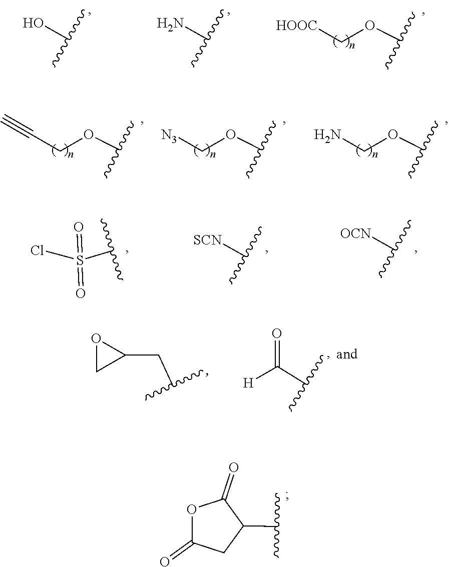

and n is between 0-6.

In one embodiment, the AMPK binding moiety is selected from the group consisting of AMPK binding moiety selected from the group consisting of:

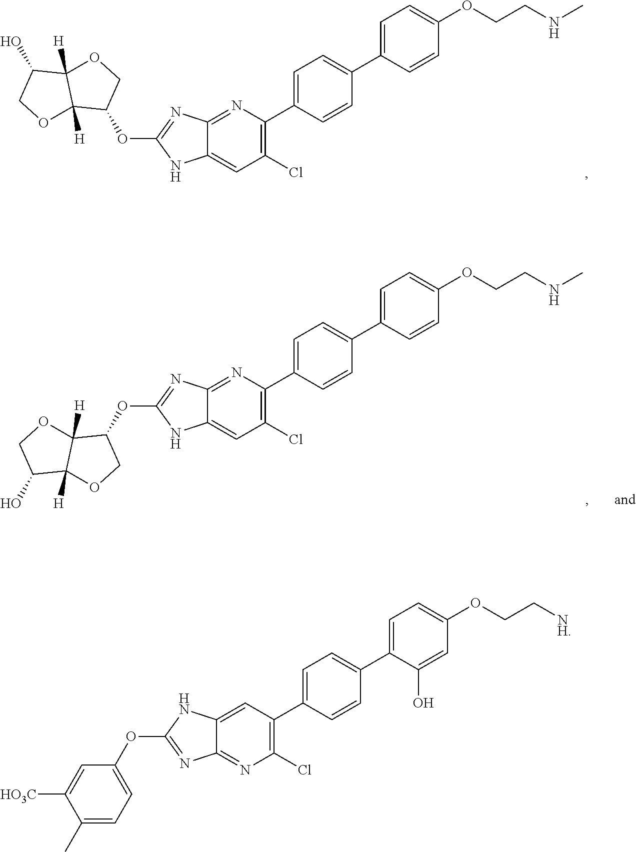

Additional AMPK kinase binding moieties that can be used in accordance with the present invention include Other AMPK activators include A769662 (Cool et al., Cell Metab. 3, 403-416 (2006)) and PT1 (Pang et al., J. Biol. Chem. 283, 16051-16060 (2008), and derivatives thereof and as further modified in accordance with the teachings detailed herein for use and optimization in the chimeric small molecules of the present invention.

AMPK binding moieties can be as described for example in U.S. Patent Publication 20050038068, incorporated herein by reference, and can be according to

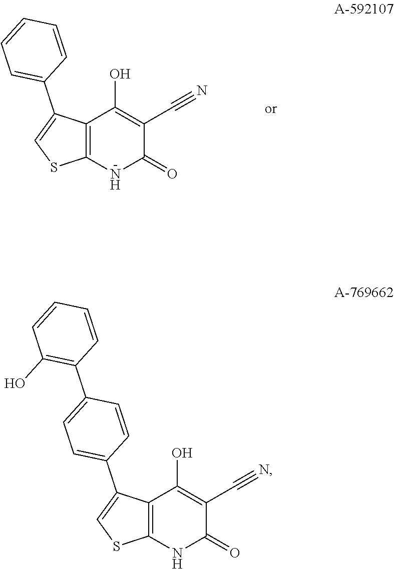 [SMILES: N#Cc1c(O)c2c(-c3ccccc3)csc2[nH]c1=O ; N#Cc1c(O)c2c(-c3ccc(-c4ccccc4O)cc3)csc2[nH]c1=O]

or derivatives thereof and as further modified in accordance with the teachings detailed herein for use and optimization in the chimeric small molecules of the present invention.

In one example embodiment, the kinase binding moiety is a AMPK activator. In an example embodiment, the AMPK activator is selected from:

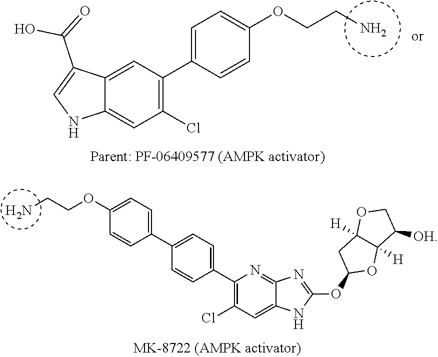 [SMILES: NCCOc1ccc(-c2cc3c(C(=O)O)c[nH]c3cc2Cl)cc1 ; NCCOc1ccc(-c2ccc(-c3nc4nc(O[C@H]5C[C@H]6OC[C@@H](O)[C@H]6O5)[nH]c4cc3Cl)cc2)cc1]

Other AMPK activators include A769662, which has a pEC₅₀ value of 6.0, see e.g. Cool, B., et al. “Identification and Characterization of a Small Molecule AMPK Activator That Treats Key Components of Type 2 Diabetes and the Metabolic Syndrome.” Cell Metabolism 2006, 3 (6), 403-416, herein incorporated by reference in its entirety.

AMPK activators can be as described for example in U.S. Patent Publication 20050038068, incorporated herein by reference, and can be according to

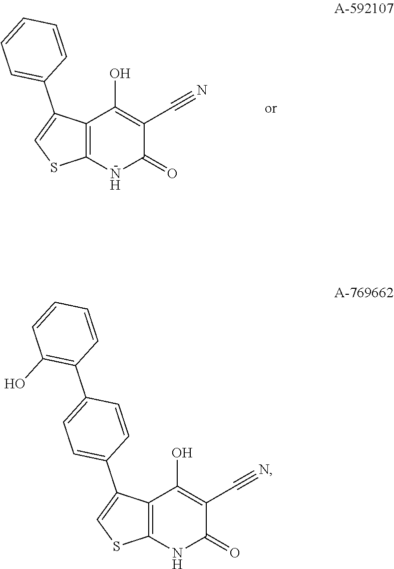 [SMILES: N#Cc1c(O)c2c(-c3ccccc3)csc2[nH]c1=O ; N#Cc1c(O)c2c(-c3ccc(-c4ccccc4O)cc3)csc2[nH]c1=O]

AMPK activators can be as described in International Patent Publications WO2007019914, WO2009124636, WO2009135580, WO2008006432, or WO2009152909, incorporated herein by reference. In one example embodiment, the activator can be according to

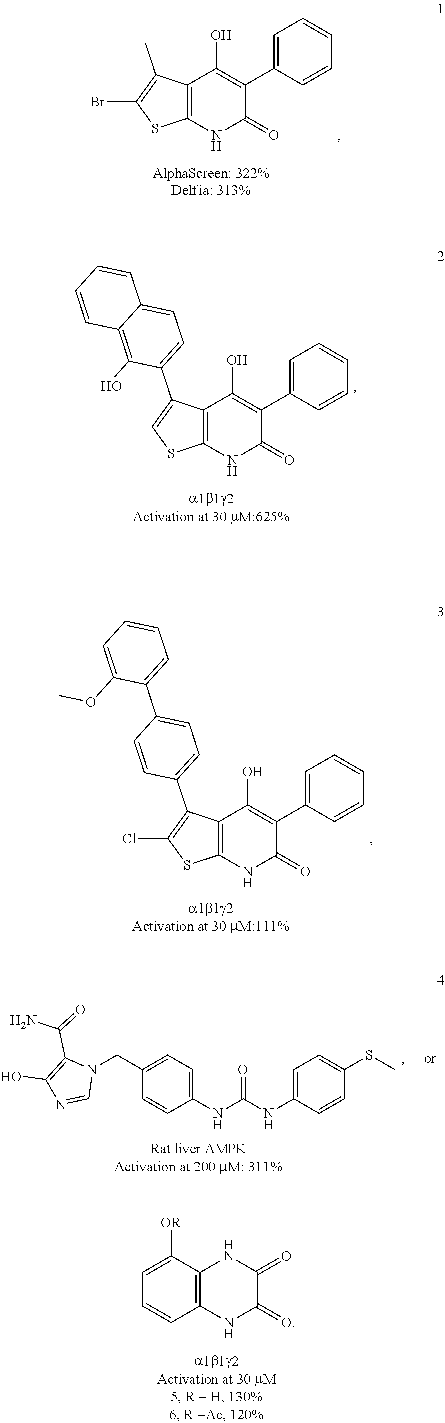

The AMPK activator can be as described in International Patent Publication WO2009100130, incorporated herein by reference. In one aspect, the AMPK activator is according to

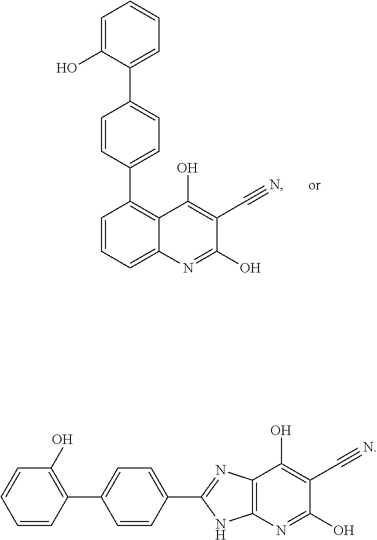 [SMILES: N#Cc1c(O)nc2cccc(-c3ccc(-c4ccccc4O)cc3)c2c1O ; N#Cc1c(O)nc2[nH]c(-c3ccc(-c4ccccc4O)cc3)nc2c1O]

The AMPK activator can be as described in International Patent Publications WO2010036613, WO2010047982, WO2010051176, WO2010051206, WO2011106273, or WO2012116145. In one example embodiment, the AMPK activator is according to

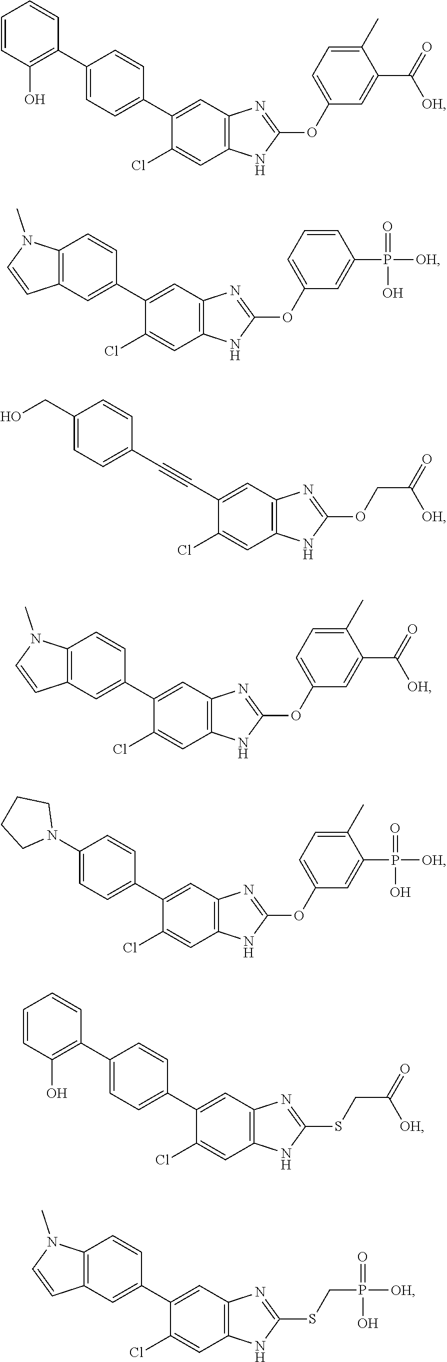

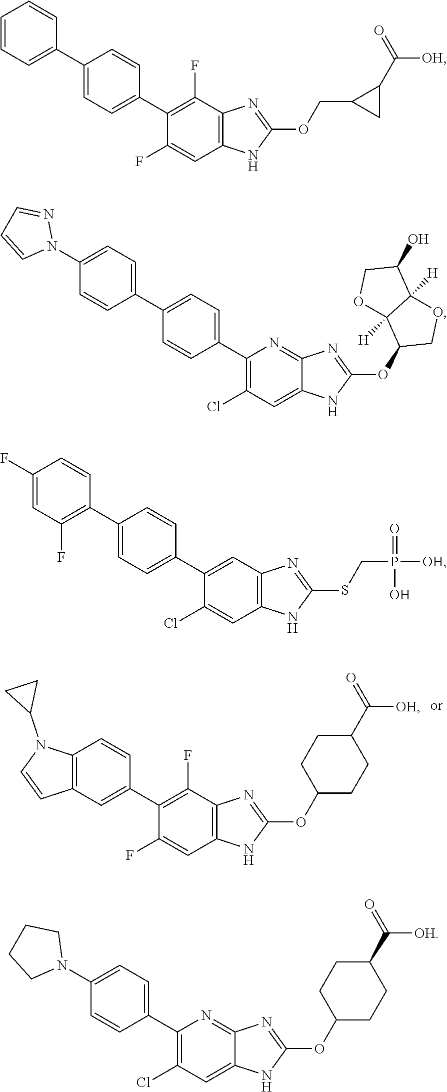

In one example embodiment, the AMPK activator can be as described in International Patent Publications WO2011029855, WO2011138307, WO2012119979, WO2012119978, incorporated herein by reference. In one aspect the AMPK activator can be selected from

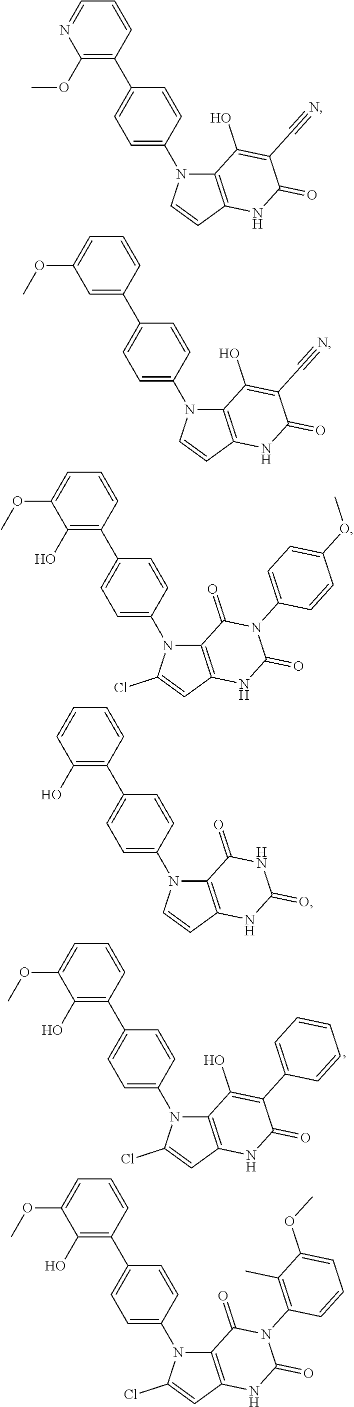

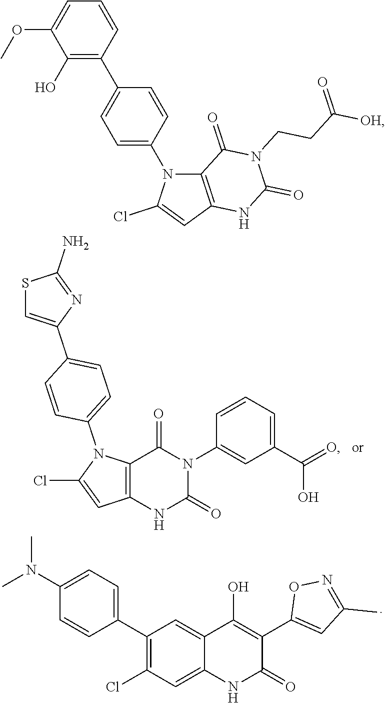

In one example embodiment, the AMPK activator can be as described in International Patent Publications WO2011032320, WO2011033099, WO2011069298, WO2011070039, WO2011128251, WO2012001020, incorporated herein by reference. In one aspect the AMPK activator can be selected from

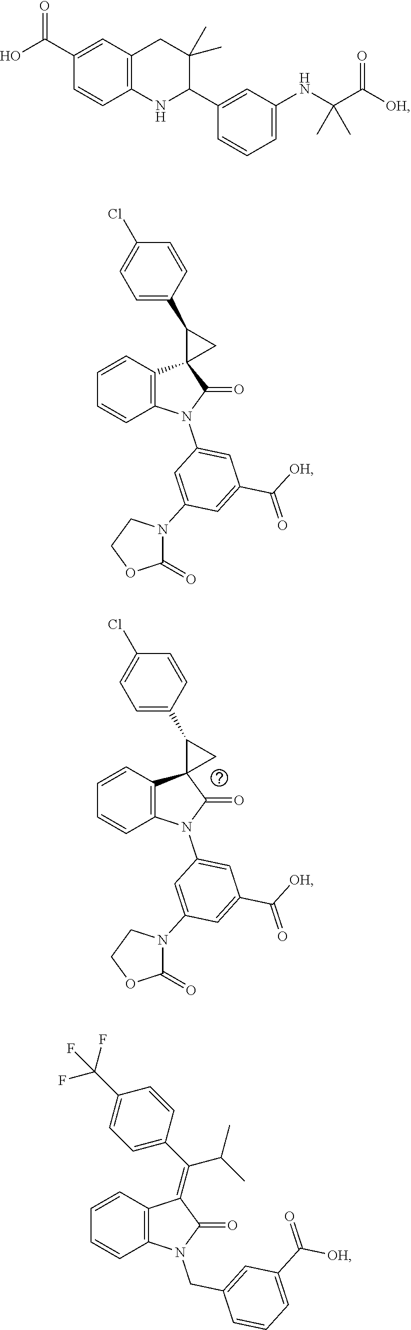 [SMILES: CC(C)(Nc1cccc(C2Nc3ccc(C(=O)O)cc3CC2(C)C)c1)C(=O)O ; O=C(O)c1cc(N2CCOC2=O)cc(N2C(=O)[C@@]3(C[C@@H]3c3ccc(Cl)cc3)c3ccccc32)c1 ; O=C(O)c1cc(N2CCOC2=O)cc(N2C(=O)[C@]3(C[C@H]3c3ccc(Cl)cc3)c3ccccc32)c1 ; CC(C)/C(=C1\C(=O)N(Cc2cccc(C(=O)O)c2)c2ccccc21)c1ccc(C(F)(F)F)cc1]

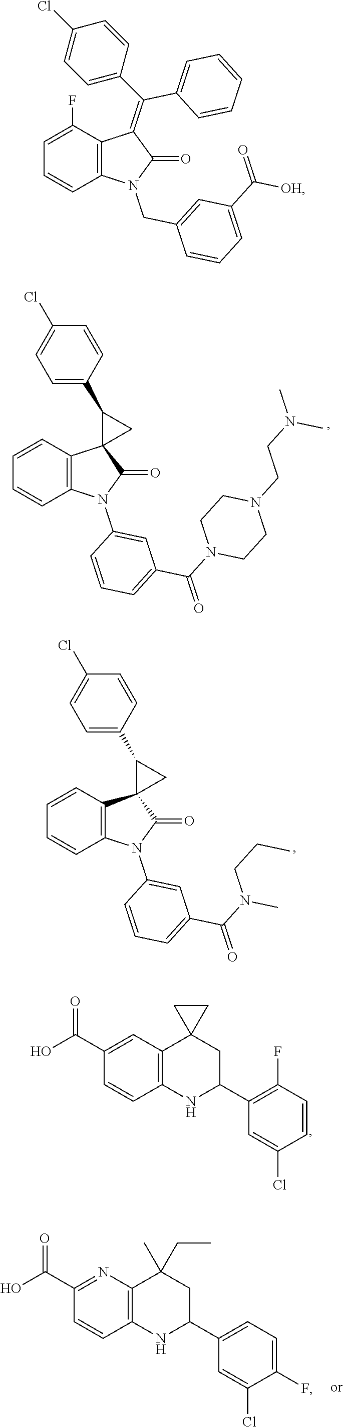 [SMILES: O=C(O)c1cccc(CN2C(=O)/C(=C(\c3ccccc3)c3ccc(Cl)cc3)c3c(F)cccc32)c1 ; CN(C)CCN1CCN(C(=O)c2cccc(N3C(=O)[C@@]4(C[C@@H]4c4ccc(Cl)cc4)c4ccccc43)c2)CC1 ; CCCN(C)C(=O)c1cccc(N2C(=O)[C@]3(C[C@H]3c3ccc(Cl)cc3)c3ccccc32)c1 ; O=C(O)c1ccc2c(c1)C1(CC1)CC(c1cc(Cl)ccc1F)N2 ; CCC1(C)CC(c2ccc(F)c(Cl)c2)Nc2ccc(C(=O)O)nc21 ; "or"]

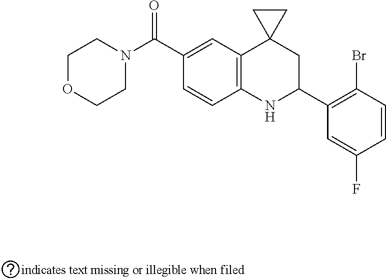 [SMILES: O=C(c1ccc2c(c1)C1(CC1)CC(c1cc(F)ccc1Br)N2)N1CCOCC1]

In one example embodiment, the AMPK activator can be as described in International Patent Publication WO2011080277, incorporated herein by reference. In one aspect the AMPK activator can be

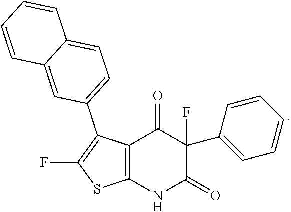 [SMILES: O=C1Nc2sc(F)c(-c3ccc4ccccc4c3)c2C(=O)C1(F)c1ccccc1]

In one example embodiment, the AMPK activator can be as described in International Patent Publication WO2012033149, incorporated herein by reference. In one aspect the AMPK activator can be selected from

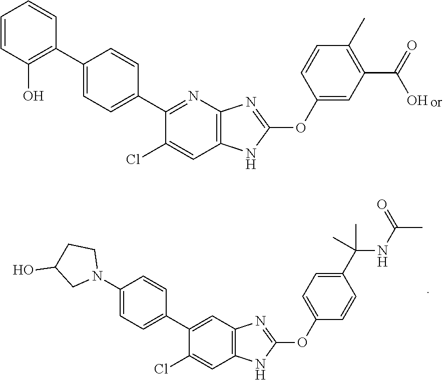 [SMILES: Cc1ccc(Oc2nc3nc(-c4ccc(-c5ccccc5O)cc4)c(Cl)cc3[nH]2)cc1C(=O)O ; CC(=O)NC(C)(C)c1ccc(Oc2nc3cc(-c4ccc(N5CCC(O)C5)cc4)c(Cl)cc3[nH]2)cc1]

In an example embodiment, the AMPK activator is MT47-100 and has the formula:

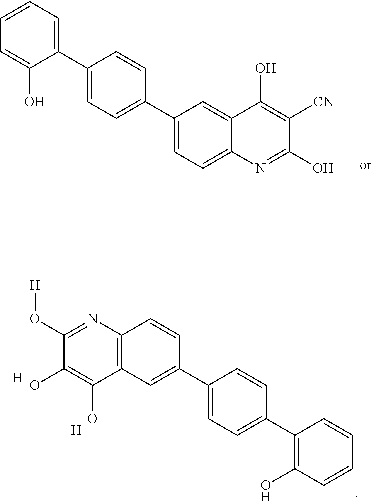

MT47-100 modulates activity of the AMPK but the direction of modulation depends on the subunit composition of the enzyme. MT47-100 acts as a direct activator of β1 subunit-containing AMPK, and as an allosteric inhibitor of β2 subunit-containing AMPK. The pKᵢ value as an activator is 5.4, while the pKᵢ value is 4.6 as an allosteric inhibitor. See e.g. Scott, J. W., et al. “Inhibition of AMP-Activated Protein Kinase at the Allosteric Drug-Binding Site Promotes Islet Insulin Release.” Chemistry & Biology 2015, 22 (6), 705-711, herein incorporated by reference in its entirety.

Additional AMPK binding moieties for use in the present invention can be as described in International Patent Publications WO2007019914, WO2009124636, WO2009135580, WO2008006432, WO2009152909, WO2011029855, WO2011138307, WO2012119979, WO2012119978, WO2011032320, WO2011033099, WO2011069298, WO2011070039, WO2011128251, WO2012001020, WO2011080277, WO2012033149 incorporated herein by reference, and derivatives thereof and as further modified in accordance with the teachings detailed herein for use and optimization in the chimeric small molecules of the present invention.

### PKC Binding Moiety

In one example embodiment, the kinase binding moiety is an PKC kinase binding moiety. In one example embodiment, the kinase binding moiety is a PKC activator or inhibitor. Protein Kinase C (PKC) is comprised of multiple isozymes and plays a role in signal transduction pathways, exhibiting a tissue-specific expression and playing a variety of biological roles. In an embodiment, the PKC kinase binding moiety can be utilized in the chimeric small molecules disclosed herein that is selective for a PKC isoform, for example classical (cPKCs-α, βI, βII, γ), novel (nPKCs-δ, ϵ, η, θ), atypical (aPKCs-ζ, τ/λ), and PKCμ (a form between novel and atypical isoforms). In one example embodiment, the PKC binding moiety is according to the formula PKC binding moiety of the formula,

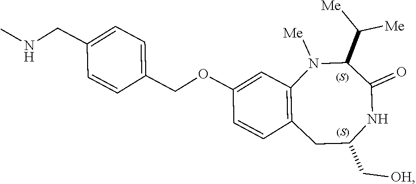

or an analog thereof.

Additional PKC binding moieties that can be configured for use in the molecules described herein are found, for example in International Patent Application PCT/US21/12816 at [1079]-[0194], incorporated specifically herein.

In one example embodiment, the kinase binding molecule can be designed as an activator of a diacylglycerol (DAG) responsive C1 domain-containing protein, such as Protein Kinase C. Protein Kinase C (PKC) is comprised of multiple isozymes and plays a role in signal transduction pathways, exhibiting a tissue-specific expression and playing a variety of biological roles. Activators of PKC can be utilized in the chimeric small molecules disclosed herein, the activating moiety is selective for a PKC isoform.

In one example embodiment, the kinase binding moiety is a DAG activator. The activator of a DAG responsive protein may comprise a DAG-indolactone as described in L. C. Garcia et al., Bioorg. Med. Chem., 22 (2014) 3123-3140. Exemplary DAG-indolactones may be according to the formula

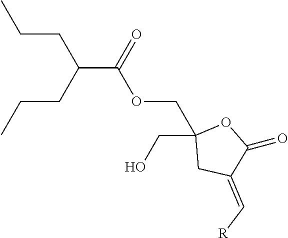

wherein R is an indole. R can be, for example, 1-methyl, 1H-indole5-yl, 1-methyl, 1H-indole6-yl, 1-methyl, 1H-indole4-yl, or. 1-methyl, 1H-indole7-yl. In one example embodiment, the compounds are selective for PKCα or PKCε.

DAG lactones, such as AJH-836, as described in Cooke, et al., J. Biol. Chem. (2018) 293(22) 8330-8341. In one example embodiment, the DAG lactone can be according to the formula

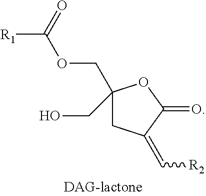

As provided in Cooke, AJH-836 formula is

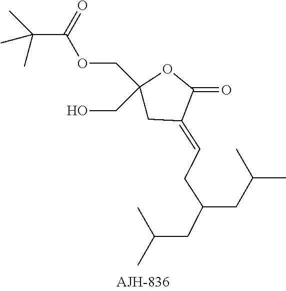 [SMILES: CC(C)CC(C/C=C1\CC(CO)(COC(=O)C(C)(C)C)OC1=O)CC(C)C]

and is selective for PKCδ and PKC.

Teleocidins, such as (−)-indolactam-V (ILV), and benzolactam-V8s, for example, 7-substituted Benzolactam-V8s, can be utilized as PKC activators. The PKC activator can be as described in Ma, et al., Org. Lett. 4:14 (2002) DOI: 10.1021/010261251.

In one example embodiment, the PKC activator is according to the formula

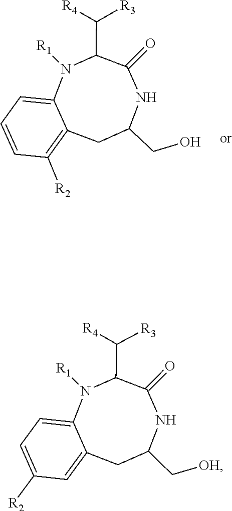

wherein R1, R3, and R4 are each independently alkyl, alkenyl, alkynyl, and R2 can be selected from divalent hydrocarbon selected from saturated or unsaturated alkylene (e.g., branched alkylelene, linear alkylene, cycloalkylene, C1-C22 branched alkylelene, C1-C22 linear alkylene, C3-C22 cycloalkylene, C1-C10 branched alkylelene, C1-C10 linear alkylene, C3-C10 cycloalkylene, C1-C8 branched alkylelene, C1-C8 linear alkylene, C3-C8 cycloalkylene), C1-C22 saturated or unsaturated heteroalkylene (e.g., branched heteroalkylelene, linear heteroalkylene, heterocycloalkylene, C1-C22 branched heteroalkylelene, C1-C22 linear heteroalkylene, C3-C22 heterocycloalkylene, C1-C10 branched heteroalkylelene, C1-C10 linear heteroalkylene, C3-C10 heterocycloalkylene, C1-C8 branched heteroalkylelene, C1-C8 linear heteroalkylene, C3-C8 heterocycloalkylene), arylene (e.g., C5-C22 arylene), heteroarylene (e.g., C5-C22 heteroarylene), or combinations thereof; wherein each of the foregoing may have one or more (e.g., two, three, four, five) points of substitution, substituted amides, including those selected from those as described in Table 1 of Kozikowski et al. J. Med. Chem, 2003, 46:3, 364-373, Table 1 at page 366, incorporated specifically herein by reference. R2 can be selected from one or more of —(C(Ra)(Ra))1-8-, —(OC(Ra)(Ra))1-8-, —(OC(Ra)(Ra)—C(Ra)(Ra))1-8-, —N(Ra)—, —O—, —C(O)—, optionally substituted C6 arylene, optionally substituted C5-12 heteroarylene, C3-6 cycloalkylene substituted with hydroxy, or C4 heterocycloalkylene substituted with hydroxy; wherein each of the foregoing may have one or more (e.g., two, three, four, five) points of substitution; and Ra is independently selected at each occurrence from hydrogen, or alkyl (e.g., C1-C7 alkyl, C1-C3 alkyl).

In one example embodiment, the formula is according to

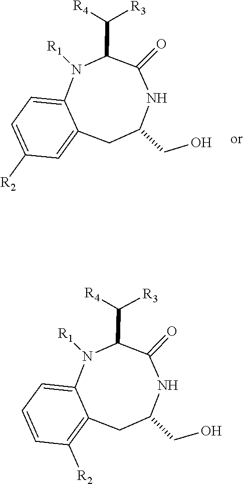

wherein R1, R3, and R4 are each independently alkyl, alkenyl, alkynyl, and R2 can be selected from divalent hydrocarbon selected from saturated or unsaturated alkylene (e.g., branched alkylelene, linear alkylene, cycloalkylene, C₁-C₂₂ branched alkylelene, C₁-C₂₂ linear alkylene, C₃-C₂₂ cycloalkylene, C₁-C₁₀ branched alkylelene, C₁-C₁₀ linear alkylene, C₃-C₁₀ cycloalkylene, C₁-C₈ branched alkylelene, C₁-C₈ linear alkylene, C₃-C₈ cycloalkylene), C₁-C₂₂ saturated or unsaturated heteroalkylene (e.g., branched heteroalkylelene, linear heteroalkylene, heterocycloalkylene, C₁-C₂₂ branched heteroalkylelene, C₁-C₂₂ linear heteroalkylene, C₃-C₂₂ heterocycloalkylene, C₁-C₁₀ branched heteroalkylelene, C₁-C₁₀ linear heteroalkylene, C₃-C₁₀ heterocycloalkylene, C₁-C₈ branched heteroalkylelene, C₁-C₈ linear heteroalkylene, C₃-C₈ heterocycloalkylene), arylene (e.g., C₅-C₂₂ arylene), heteroarylene (e.g., C₅-C₂₂ heteroarylene), or combinations thereof; wherein each of the foregoing may have one or more (e.g., two, three, four, five) points of substitution, substituted amides, including those selected from those as described in Table 1 of Kozikowski et al. J. Med. Chem, 2003, 46:3, 364-373, Table 1 at page 366, incorporated specifically herein by reference.

R2 can be selected from one or more of —(C(R^{a})(R^{a}))₁₋₈—, —(OC(R^{a})(R^{a}))₁₋₈—, —(OC(R^{a})(R^{a})—C(R^{a})(R^{a}))₁₋₈—, —N(R^{a})—, —O—, —C(O)—, optionally substituted C₆ arylene, optionally substituted C₅₋₁₂ heteroarylene, C₃₋₆ cycloalkylene substituted with hydroxy, or C₄ heterocycloalkylene substituted with hydroxy; wherein each of the foregoing may have one or more (e.g., two, three, four, five) points of substitution; and R^{a} is independently selected at each occurrence from hydrogen, or alkyl (e.g., C₁-C₇ alkyl, C₁-C₃ alkyl).

In one example embodiments, the formula is according to

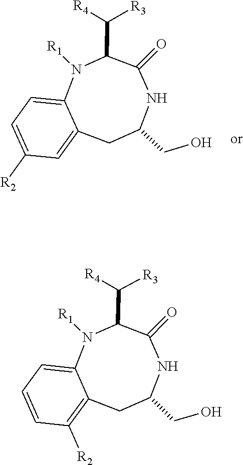

wherein R1, R3 and R4 is independently alkyl, alkenyl, alkylnyl, and R2 can be selected from. In one example embodiment, the PKC activator is a benzolactam analogue of ILV, with R can be CC(CH₂)₇CH₃ or (CH₂)₉CH₃, as described in Kozikowski et al., J. Med. Chem., 1997, 40:9 1316-1326.

In one example embodiment, R1, R3 and R4 are alkyl, in some embodiments, R1, R3 and R4 are methyl. In one example embodiment, the formula is according to:

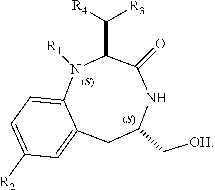

In one example embodiment, the PKC activator is a natural product activator, for example, DPP, prostratin, mezerein, octahydromexerein, thymeleatoxin, (−)-ocytlindolactam V, OAG, or resiniferatoxin, as described in Kazanietz. et al., Mol. Pharma. 44:296-307 (1993).

In one example embodiment the PCK binding moiety is according to the formula PKC binding moiety of the formula,

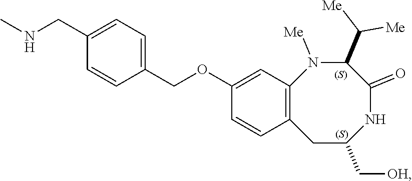

or an analog thereof.

In one example embodiments, the PKC activator is selective for PKCδ. In one example embodiment, the PKC activator is 7α-acetoxy-6β-benzoyloxy-12-Obenzoylroyleanone (Roy-Bz) as described in Bessa et al., Cell Death and Disease (2018) 9:23.

The PKC activator may be an ILV derivative, such as n-hexyl ILV, or a 10 membered ring1-Hexylindolactam-V10, or a derivative thereof, as described in Yanagita, et al., J. Med. Chem., 2008, 51:1, 46-56, incorporated herein by reference. The PKC activator may be

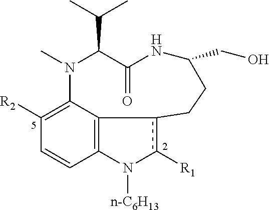

wherein R1 and R2=H, R1=H and R2=Cl, or R1=Br and R2=H, and may, in some instance be PKCδ, PKCε or PKCη.

In one example embodiment, the activator moiety is 6-Chloro-5-[4-(1-hydroxycyclobutyl)phenyl]-1H-indole-3-carboxylic Acid (PF-06409577), a benzolactam, DPP, Prostratin, Mezerein, Octahydromezerein, Thymeleatoxin, (−)-Indolactam V, (−)-Octylindolactam V, OAG, or derivatives thereof.

In one example embodiment, the activator moiety is a thieno[2,3-b]pyridine, a thienopyridone, a quinoxalinedione, a imidazo[4,5-b]pyridine, a [2,3-d]pyridine, a benizimidazole, a pyrrolo[2,3-d]pyrimidine, a spirocyclic indolinone, a tetrahydroquinoline, a thieno[2,3-b]pyridinedione, and derivatives thereof. See Expert Opin Ther. Patents (2012)22(12), incorporated herein by reference.

In other example embodiments, the PKC activators may be selected from Table 1 from PCT/US2021/012816 herein incorporated by reference.

### FKBP Binding Moiety

In one example embodiment, the kinase binding moiety is an FKBP kinase binding moiety. In one example embodiment, the kinase binding moiety can be designed as an activator or inhibitor of an FK506-binding protein (FKBP). FKBP belongs to the immunophilin family. FKBPs are present in all eukaryotes, ranging from yeasts to humans, and expressed in most tissues. Mammalian FKBPs can be subdivided into four groups: the cytoplasmic, endoplasmic reticulum, nuclear, and TPR (tetratricopeptide repeats)-containing FKBPs. In one example embodiment, the FKBP is FKBP12, which binds to intracellular calcium release channels and TGF-β type I receptor. In one example embodiment, the FKBP activator moiety is of the formula

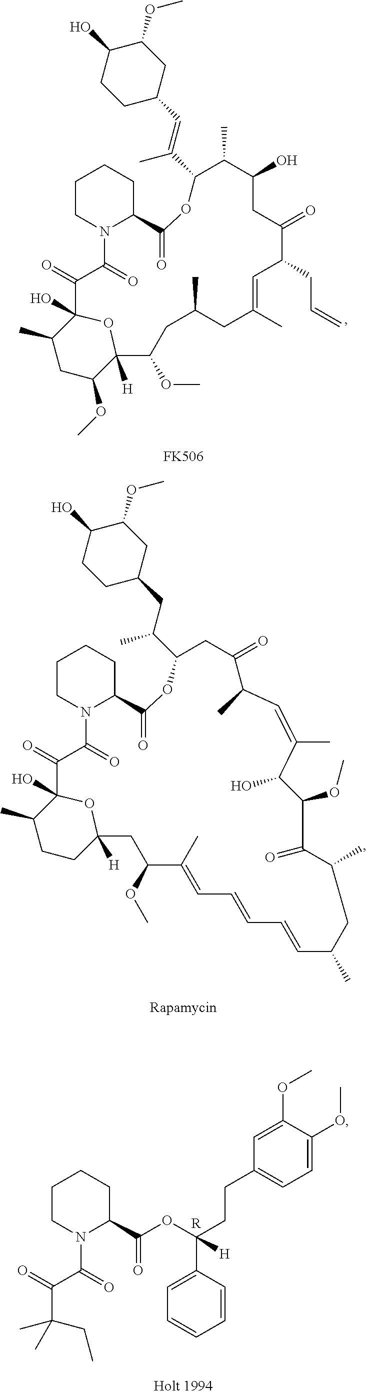

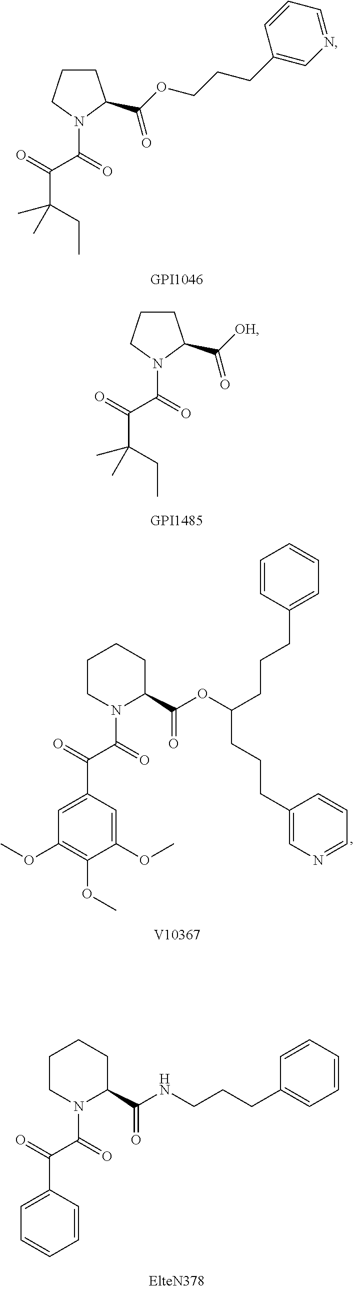 [SMILES: CCC(C)(C)C(=O)C(=O)N1CCC[C@H]1C(=O)OCCCc1cccnc1 ; CCC(C)(C)C(=O)C(=O)N1CCC[C@H]1C(=O)O ; COc1cc(C(=O)C(=O)N2CCCC[C@H]2C(=O)OC(CCCc2ccccc2)CCCc2cccnc2)cc(OC)c1OC ; O=C(C(=O)N1CCCC[C@H]1C(=O)NCCCc1ccccc1)c1ccccc1]

and any derivative thereof. See e.g. (Kolos et al. FKBP Ligands—Where We Are and Where to Go? Front. (2018) FKBP Ligands—Where We Are and Where to Go? Front. Pharmacol. 9:1425.) IRTK Binding Moiety

In one example embodiment, the kinase binding moiety is an IRTK kinase binding moiety. The insulin receptor (IR) is a hetero-tetrameric protein consisting of two extracellular a subunits and two transmembrane β subunits. The binding of a ligand to the α subunit of the IR induces conformational changes in the receptor. As a result, the tyrosine kinase activity intrinsic to the β subunit of the IR is stimulated. (Salituro G M et al. Discovery of a small molecule insulin receptor activator. Recent Prog Horm Res. 2001; 56:107-26.) In one example embodiment, the kinase binding moiety is an IR activator or inhibitor. In one example, the activator for IRTK is kojic acid, or a derivative thereof.

In one example embodiment, the target is an Androgen Receptor. In one example embodiment, the localizing moiety may comprise enzalutamide. In one example embodiment, the enzalutamide is attached via an ether bond to a linker comprising an azide end. Thus, in one example embodiment, the addition of alkyne functionality on the activator moiety will enable connection via biorthogonal click-chemistry. In one example embodiment, the Insulin Receptor is according to the formula:

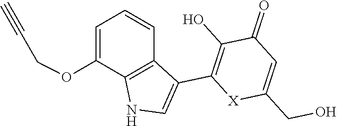

wherein X is C, N, O, S or P. In other example embodiments the IRTK activator is according to the formula:

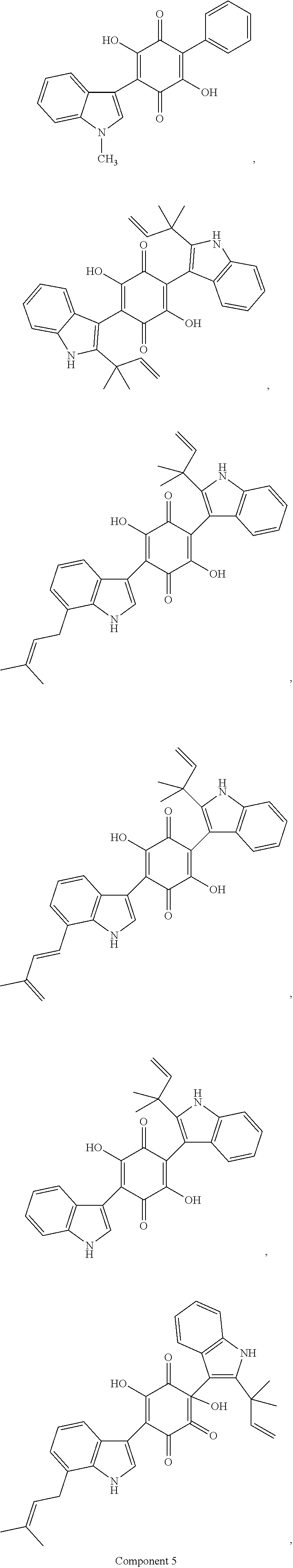

and any derivative thereof.

In one example embodiment, the IRTK activator is XMetA, also known as XOMA-159, which is a monoclonal antibody and allosteric partial agonist of the insulin receptor. See e.g. Bedinger D. H., et al. “Differential Pathway Coupling of the Activated Insulin Receptor Drives Signaling Selectivity by XMetA, an Allosteric Partial Agonist Antibody.” J Pharmacol Exp Ther 2015, 353 (1), 35-43.

### Lyn Binding Moiety

In one example embodiment, the kinase binding moiety is an Lyn kinase binding moiety. The Lyn kinase belongs to the Src-family of kinases and is the predominant Src kinase in B cells. The regulatory properties of Lyn play a role in the function of the immune system. See e.g. Xu Y., “Lyn Tyrosine Kinase.” Immunity 2005, 22 (1), 9-18. In one example embodiment, the Lyn binding moiety is an activator or inhibitor. In an example embodiment, the Lyn activator is tolimidone, also known as MLR-1023, according to the formula:

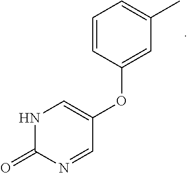

Tolimidone is a selective allosteric activator of Lyn kinas and was developed for the treatment of type 2 diabetes. Experiments with knockout mice revealed tolimidone did not lower glucose when Lyn kinase was absent. Currently, tolimidone is in a Phase 2 study in patients suffering from uncontrolled Type 2 Diabetes. Tolimidone has a pEC₅₀ of 7.2. See e.g. Saporito, M. S., et al. “MLR-1023 Is a Potent and Selective Allosteric Activator of Lyn Kinase In Vitro That Improves Glucose Tolerance In Vivo.” J Pharmacol Exp Ther 2012, 342 (1), 15-22, with the following comparison of activities in cellular and enzyme assays references below and incorporated herein by reference:

Comparison of activities of MLR-1023 and reference compounds
in cellular and enzyme [symbol] MLR-1023 was [symbol] .

| Assay | Control | MLR- [symbol] M) | [symbol] Compound [symbol] | [symbol] Compound [symbol] M) |
|---|---|---|---|---|
| Adipocyte differentation [symbol] % differentiation | 4.5 [symbol] 2 | 16.7 [symbol] 2.8 [symbol] | [symbol] 3 [symbol] | R [symbol] (10 [symbol] M) |
| PPA [symbol] fold activation | [symbol] 0.1 | 1.6 [symbol] 0.3 | 22.0 [symbol] 0.4 | [symbol] (10 [symbol] M) |
| PPA [symbol] fold activation | 1.0 [symbol] 0.2 | 1. [symbol] | [symbol] 2 [symbol] 0. [symbol] | [symbol] brate (100 [symbol] M) |
| PPA [symbol] fold activation | 1.0 [symbol] 0.1 | 0. [symbol] | [symbol] 0 [symbol] 0.8 | [symbol] (100 [symbol] M) |
| Adipo [symbol] production [symbol] ml | 10 [symbol] 3 | 1 [symbol] 2 [symbol] 11 [symbol] | 1 [symbol] 87 [symbol] | [symbol] (10 [symbol] M) |
| [symbol] % inhibition | 0. [symbol] 0.4 | 0. [symbol] | [symbol] 7 [symbol] 0. [symbol] | [symbol] ( [symbol] M) |
| Insulin [symbol] | [symbol] 0.1 | [symbol] 4 [symbol] 0.0 [symbol] | 8.0 [symbol] 0.01 | GLP [symbol] M) |
| [symbol] % inhibition | 0. [symbol] .2 | 14. [symbol] 0. [symbol] | [symbol] 3 [symbol] 0.19 | [symbol] mine [symbol] M) |
| GLP [symbol] | 17 [symbol] 1.1 | 29 [symbol] 1.2 | 157 [symbol] 11 [symbol] | GLP [symbol] M) |

[symbol]

< [symbol] value [symbol]
indicates data missing or illegible when filed

### PK Binding Moiety

In one example embodiment, the kinase binding moiety is a PK kinase binding moiety. Pyruvate kinase (PK) catalyzes the transphosphorylation from phosphoenolpyruvate (PEP) to ADP to generate ATP in glycolysis. PK is expressed in four different isozymic forms: L, R, M1, and M2 in mammalian tissues depending upon the metabolic requirement and their regulatory properties. The M2, L, and R isozymes have homotropic cooperative activation with PEP and heterotropic cooperative activation with FBP. See e.g. Gupta V., et al. “Human Pyruvate Kinase M2: A Multifunctional Protein.” Protein Science 2010, 19 (11), 2031-2044.

In one example embodiment the PK kinase binding moiety is a PK activator. In an example embodiment, the PK activator is Mitapivat, also known as AG-348, according to the formula: with the following properties

| | |
|---|---|
| Hydrogen bond acceptors | 7 |
| Hydrogen bond donors | 1 |
| Rotatable bonds | 7 |
| Topological polar surface area | [symbol] 0.99 |
| Molecular weight | [symbol] 60.17 |
| XLogP | 2.75 |
| No. Lipinski's rules broken | 0 |

[symbol]

indicates data missing or illegible when filed

Mitapivat is a small molecule allosteric activator of the pyruvate kinases. It activates the PK isoform that is found in erythrocytes, PKR protein that is expressed from the PKLR gene, and the embryonic PKM2 isoform, expressed from the PKM gene. Mitapivat was developed as a novel therapy for diseases of red blood cells that are associated with inherited PKR deficiency, and for cancer therapy via activation of PKM2. Activation of PK in red cells increases hemoglobin levels. The active drug is the sulfate hydrate. Mitapivat has an pEC₅₀ value of >7.0 for PKM2. In one example embodiment, the PK activator is any compound from U.S. Pat. No. 8,785,450B2 herein incorporated by reference, or any derivative thereof. In one example embodiment, the PK activator is any compound from International Patent Publication WO2013056153A1, herein incorporated by reference, or any derivative thereof.

In one example embodiment, the kinase binding moiety is a PK inhibitor, see above for more information regarding PK kinase. In an example embodiment, the PK inhibitor is any identified in U.S. Pat. No. 6,534,501, herein incorporated by reference, or any derivative thereof.

### NOP Binding Moiety

The nociceptin opioid peptide (NOP) receptor is part of the opioid receptor family of GPCRs, which couples to Gi/Go and inhibits adenylate cyclase activity. In one example embodiment, the kinase binding moiety or target binding moiety binds to a GPCR opioid receptor. In one example embodiment, the kinase binding moiety is a NOP activator. In an example embodiment, the NOP activator has any of the following formulas:

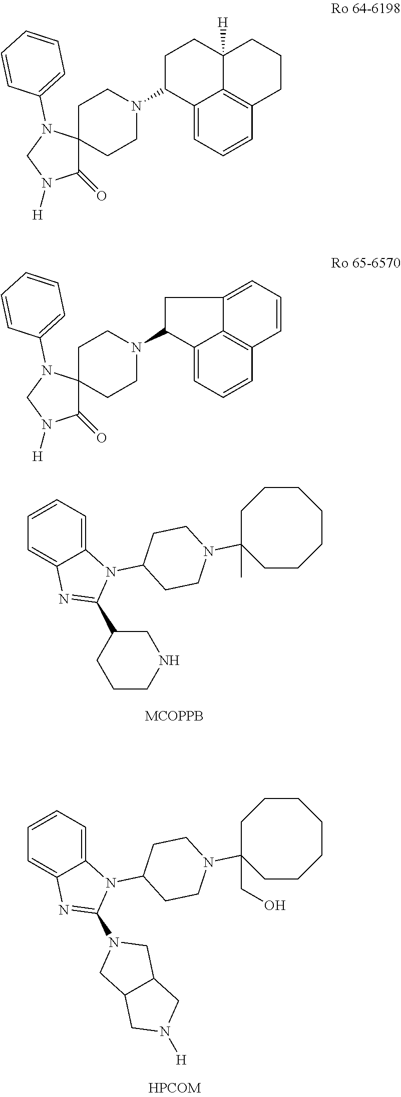

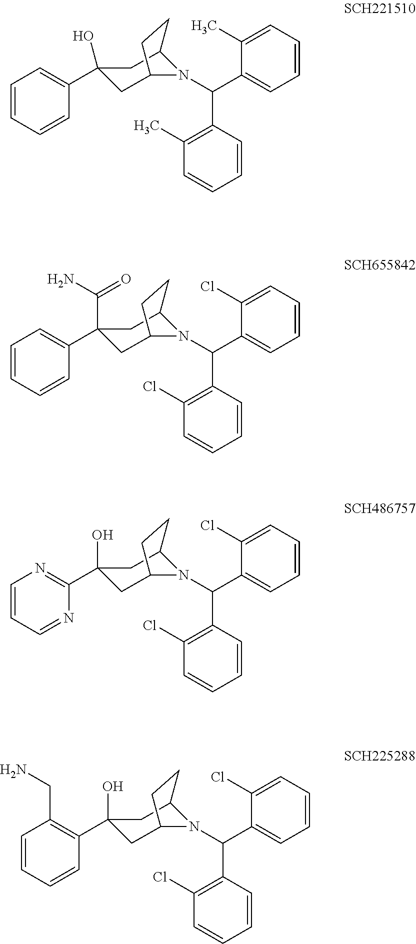 [SMILES: Cc1ccccc1C(c1ccccc1C)N1C2CCC1CC(O)(c1ccccc1)C2 ; NC(=O)C1(c2ccccc2)CC2CCC(C1)N2C(c1ccccc1Cl)c1ccccc1Cl ; OC1(c2ncccn2)CC2CCC(C1)N2C(c1ccccc1Cl)c1ccccc1Cl ; NCc1ccccc1C1(O)CC2CCC(C1)N2C(c1ccccc1Cl)c1ccccc1Cl]

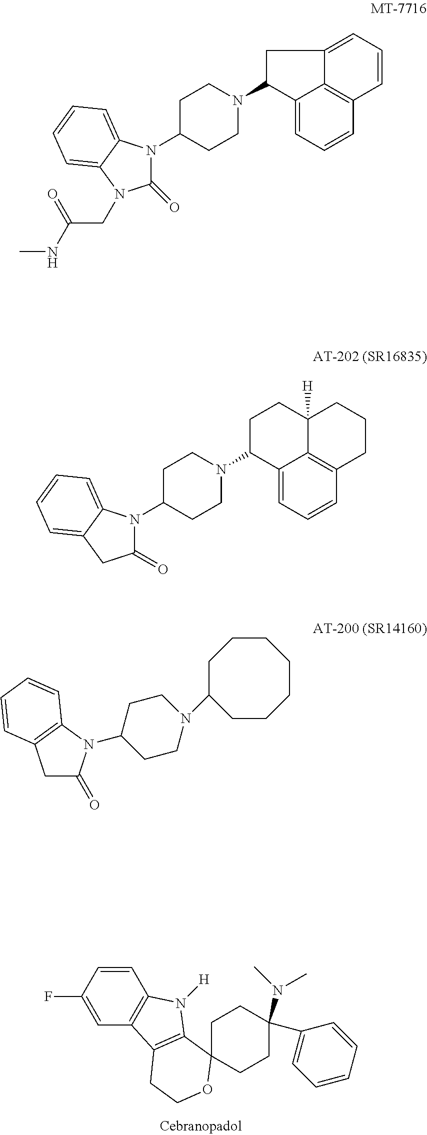

or any derivative thereof.

In an example embodiment, the NOP activator is the NOP agonist Ser100 according to the formula: Ac-RYYRWKKKKKKK-NH2 (SEQ ID NO: 1). In an example embodiment, the NOP activator is the NOP agonist N/OFQ according to the formula: FGGFTGARKSARKLANQ (SEQ ID NO: 2). In an example embodiment the NOP activator is JNJ-19385899, see e.g. Zaveri, N. T. “Nociceptin Opioid Receptor (NOP) as a Therapeutic Target: Progress in Translation from Preclinical Research to Clinical Utility.” J. Med. Chem. 2016, 59 (15), 7011-7028, herein incorporated by reference in its entirety.

A number of proteins such as G protein-coupled receptor kinases, β-arrestins and G proteins clearly regulate NOP receptor functions. It has also been shown sodium and guanyl nucleotides can modify the functional NOP complex and G protein interaction. Other G protein-coupled receptors, such as mu-opioid receptors, appear to be able to form heterodimers with NOP receptors, potentially modifying the receptor protein, see e.g. Wang, H.-L., et al. “Heterodimerization of Opioid Receptor-like 1 and μ-Opioid Receptors Impairs the Potency of μ Receptor Agonist.” Journal of Neurochemistry 2005, 92 (6), 1285-1294.

In an embodiment, the binder is an allosteric regulator of the delta opioid receptor. In an embodiment, the binder is

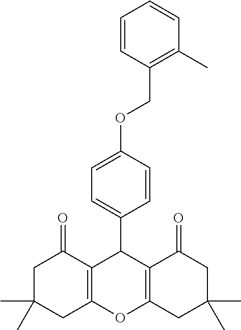

BMS-986187, 3,3,6,6-tetramethyl-9-[4-[(2-methylphenyl)methoxy]phenyl]-4,5,7,9-tetrahydro-2H-xanthene-1,8-dione.

In an embodiment, the binder is an allosteric regulator of the mu opioid receptor.

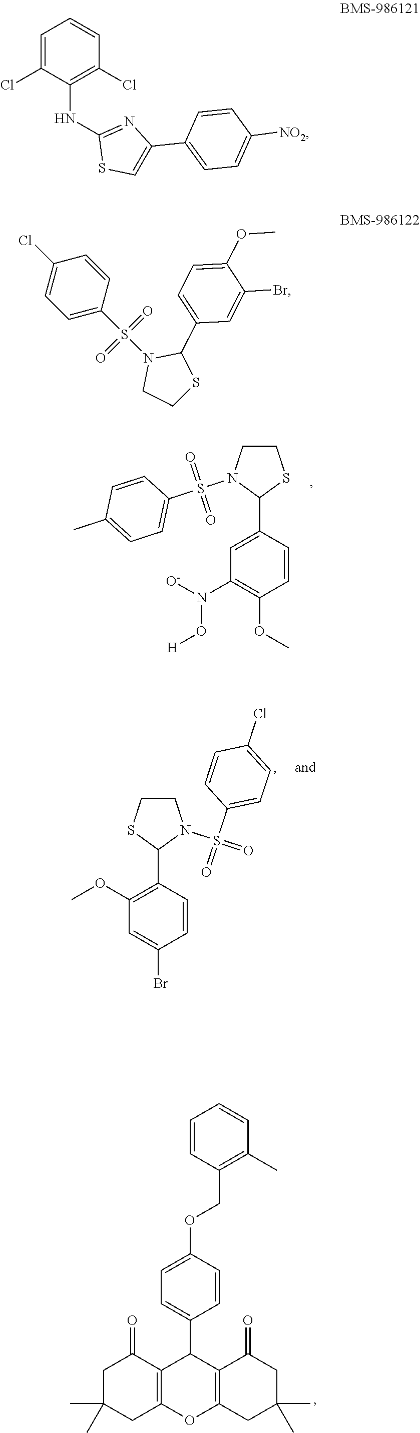

which may be referenced as BMS-986121 [(4-{2-[(2,6-dichlorophenyl)amino]-1,3-thiazol-4-yl}phenyl)(hydroxy)imino]-21-oxidanyl; BMS-986122 2-(3-bromo-4-methoxyphenyl)-3-(4-chlorophenyl)sulfonyl-1,3-thiazolidine; BMS-986123 [hydroxy({2-methoxy-5-[3-(4-methylbenzenesulfonyl)-1,3-thiazolidin-2-yl]phenyl})imino]-21-oxidanyl; BMS-986124 2-(4-bromo-2-methoxyphenyl)-3-(4-chlorobenzenesulfonyl)-1,3-thiazolidine; or BMS-986187 3,3,6,6-tetramethyl-9-[4-[(2-methylphenyl)methoxy]phenyl]-4,5,7,9-tetrahydro-2H-xanthene-1,8-dione, respectively.

In one example embodiment, the NOP binder is a NOP antagonist. In an example embodiment, the NOP antagonist has any of the following formulas:

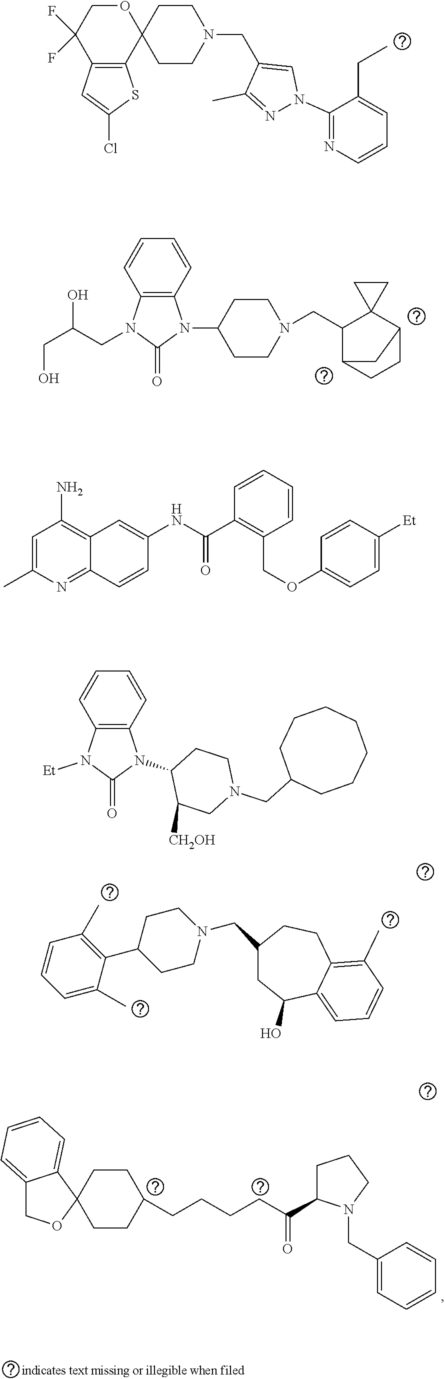

see Zaveri J. Med. Chem. 2016.

### MAPK Binding Moiety

In one example embodiment, the kinase binding moiety is a mitogen-activated protein kinase (MAPK) binding moiety. In one example embodiment, the MAPK binding moiety is an inhibitor or activator. MAPK is involved in the signal-transduction pathways. A common feature of MAPKs is their ability to phosphorylate the transactivation domains of transcription factors and, as a result, modulate transcriptional activity. In one example embodiment, the kinase binding moiety is a MAPK inhibitor.

In an example embodiment, the MAPK inhibitor is a p38α MAPK inhibitor comprising:

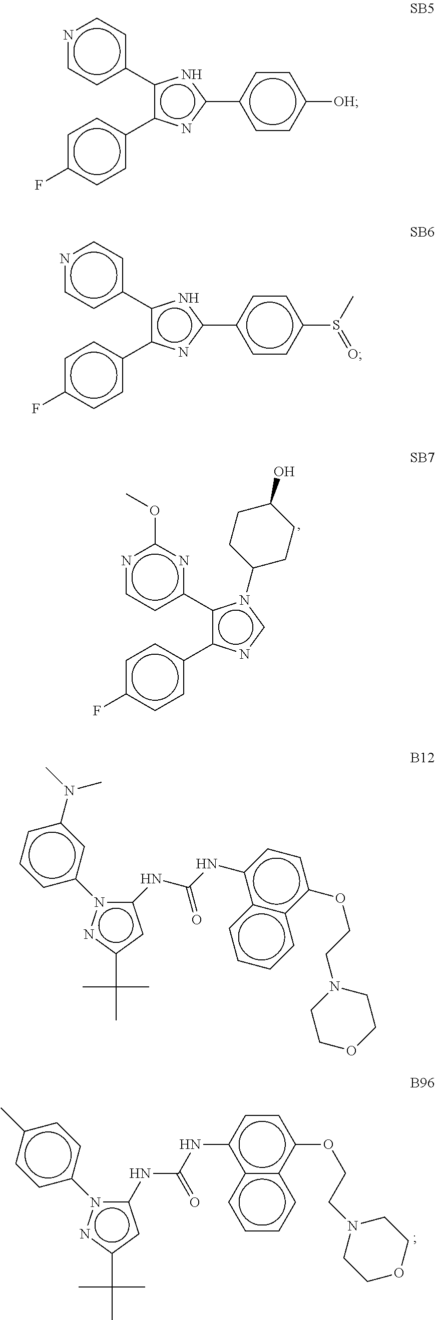

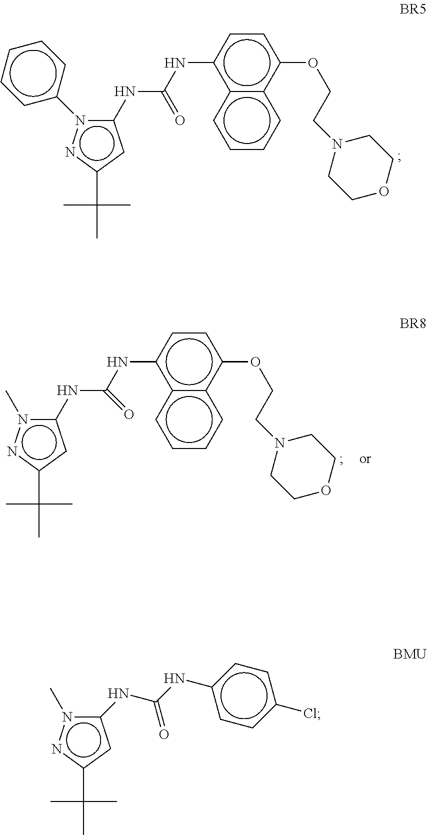

and derivatives thereof, which can be utilized as activating moieties in the chimeric small molecules of the invention. Inhibitor B96 may also be known as Doramapimod, which is an allosteric inhibitor. Doramapimod shows moderate selectivity for the p38alpha, -beta and -gamma isozymes compared to p38delta. It shows moderate selectivity for the p38α, -β and -γ isozymes compared to p38δ. A Kd value of 0.1 nM is reported, and in a screening panel of kinases, doramapimod inhibited many kinases with IC50 values <100 nM. Doramapimod has been shown to block TNFα release in LPS-stimulated THP-1 cells with an IC50 value of 18 nM. Doramapimod inhibits MAPK14 with pKd of 9.4 and pIC50 of 7.7, MAPK11 pIC50 of 8.1, MAPK12 pIC50 of 7.5, and MAPK13 pIC50 of 6.5 See, Moffett, et al., Bioorg. Med. Chem. Lett. 2011, 21, 7155-7165. Further areas for modification when tailoring the molecule for use in the chimeric small molecules are described in Moffett, in particular at FIG. 3 and its associated teachings incorporated by reference. In an aspect, the molecule incorporates non-aromatic fragments which make productive hydrogen bond interactions with Arg 70 on the αC-helix.

In an embodiment, the MAPK inhibitor is the allosteric inhibitor of p38 according to compound 10, which is discussed in further detail in the context of Jnk-1.

In an embodiment, the MAPK inhibitor is SB203580 (SB6). In an embodiment, the MAPK inhibitor is Skepinone-L, with the formula

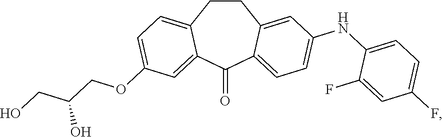 [SMILES: O=C1c2ccc(Nc3ccc(F)cc3F)cc2CCc2ccc(OC[C@H](O)CO)cc21]

and its derivatives. In an embodiment, the MAPK inhibitor is Sorafenib, with the formula, and its derivatives.

In one example embodiment, the MAPK inhibitor is the small molecule KC-706.

### EGFR Binding Moiety

In one example embodiment, the kinase binding moiety is an EGFR binding moiety. In one example embodiment, the EGFR binding moiety is an inhibitor or activator. EGFR, is a tyrosine kinase receptor belonging to the ErbB family of receptors that mediates cell growth, differentiation and repair in cells, especially non-cancerous cells.

In one embodiment, the EGFR binding molecule is of the formula,

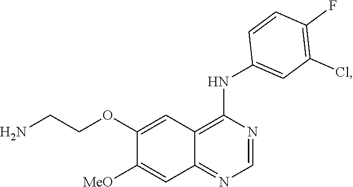 [SMILES: COc1cc2ncnc(Nc3ccc(F)c(Cl)c3)c2cc1OCCN]

or an analog thereof.

In an example embodiment, the EGFR binding molecule is Gefitinib. Gefitinib selectively binds to the ATP-binding site of EGFR thereby causing inhibition. In one example embodiment, the EGFR binding molecule may be any from the group comprising of Erlotinib, Afatinib, Osimertinib, Lapatinib, Neratinib, Dacomitinib, or any derivatives thereof.

In an embodiment, the kinase is an EGFR mutant. In an embodiment, the EGFR mutant comprises L858R, C797S, T790M, V984R, or a combination thereof.

In an example embodiment, the EGFR inhibitor is EAI001, was designed to overcome clinically acquired EGFR T790M/C797S mutant resistance in NSCLC by binding outside the ATP. EAI001 binds to the allosteric MT3 site of EGFR with the carboxamide forming a hydrogen bond with Asp 855, and Phenyl group forms hydrophobic interactions with the DFG-in pocket and the 1-oxoisoindolinyl extends to the solvent-accessible region. EAI001 is according to the formula:

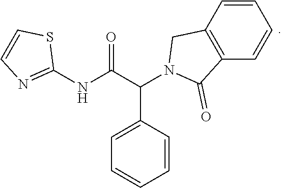 [SMILES: O=C(Nc1nccs1)C(c1ccccc1)N1Cc2ccccc2C1=O]

In an example embodiment, the EGFR inhibitor is an EAII001 analog, for example, EAI045 according to the formula:

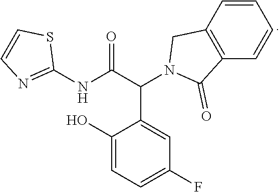 [SMILES: O=C(Nc1nccs1)C(c1cc(F)ccc1O)N1Cc2ccccc2C1=O]

EAI001 and its analogue EAI045 both exhibit potent inhibitory activity against EGFR L858R/T790M with IC50 values of 24 and 3 nm, respectively. EAI045 is an allosteric inhibitor of mutant forms of the EGFR found in lung cancers whilst sparing the wild-type receptor, and inhibits L858R/T790M mutant EGFR with an IC₅₀ of 3 nM and is >1000-fold selective for this mutant compared to wild-type receptor. Additional EGFR and its mutants and IC50 for EAI045 are:

| | | | | |
|---|---|---|---|---|
| IC₅₀ & Target | EGFR | EGFR^{L858R} | EGFR^{T790M} | EGFR^{L858R/T790M} |
| | 1.9 μM (IC₅₀) | 0.019 μM (IC₅₀) | 0.19 μM (IC₅₀) | 0.002 μM (IC₅₀) |

In screening panels EAI045 did not inhibit any other kinases by >20% (at 1000 nM EAI045), or show any liability against non-kinase targets, and in xenograft models EAI045 is effective against EGFR (L858R/T790M/C797S) tumors, a mutation profile that is resistant to all currently available ATP-competitive EGFR tyrosine kinase inhibitors. See, Angew. Chem. Int. Ed. 2020, 59, 13764-13776, incorporated herein by reference. EAI1045 shows the following properties:

| | |
|---|---|
| Hydrogen bond acceptors | 5 |
| Hydrogen bond donors | 2 |
| Rotatable bonds | 5 |
| Topological polar surface area | 110.77 |
| Molecular weight | 383.07 |
| XLogP | 1. [symbol] |
| No. Lipinski's rules broken | 0 |

[symbol]

indicates data missing or illegible when filed

In an aspect, the EGFR inhibitor is designed to overcome acquired resistance to current EGFR tyrosine kinase inhibitors which bind to the ATP pocket of the enzyme, which is the location of the many identified resistance mutations.

In an example embodiment, the EGFR inhibitor is an analog that comprises by addition of phenylpiperazine substituent on the isoindolinone ring of EAI045. The analog may be JBJ-04-125-02 according to the formula:

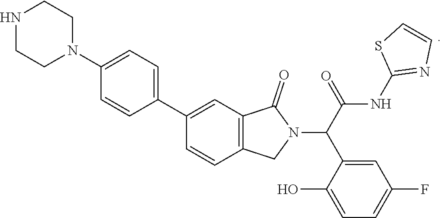 [SMILES: O=C(Nc1nccs1)C(c1cc(F)ccc1O)N1Cc2ccc(-c3ccc(N4CCNCC4)cc3)cc2C1=O]

In an embodiment, JBJ-04-125-02 exhibits sub-nanomolar potency against EGFR L858R/T790M kinase with an biochemical IC50 value of 0.26 nm. Notably, it potently inhibits cell proliferation and EGFR L858R/T790M/C797S signaling in vitro and in vivo as a single agent. X-ray crystal structure of JBJ-04-125-02 and EGFR T790M demonstrates that it binds to the allosteric site of EGFR in a similar manner to EAI001. In an aspect, JBJ-04-125-02 at (0.01-10 uM) inhibited EGFR phosphorylation and demonstrated mutant selectivity by inhibiting mutant EGFR and downstream AKT and ERK1/2 phosphorylation. Angew. Chem. Int. Ed. 2020, 59, 13764-13776, incorporated herein by reference in its entirety; see See, e.g. To et al., Single and dual targeting of mutant EGFR with an allosteric inhibitor, Cancer Discov. 2019 July; 9(7): 926-943. Doi: 10.1158/2159-8290.CD-18-0903.

In an example embodiment, the EGFR inhibitor is an inhibitor or derivative thereof identified in U.S. Pat. No. 8,242,080, herein incorporated by reference. In an example embodiment, the EGFR inhibitor is Dacomitinib, Mobocertinib, or any derivatives thereof.

In an example embodiment, the EGFR inhibitor is according to the formula:

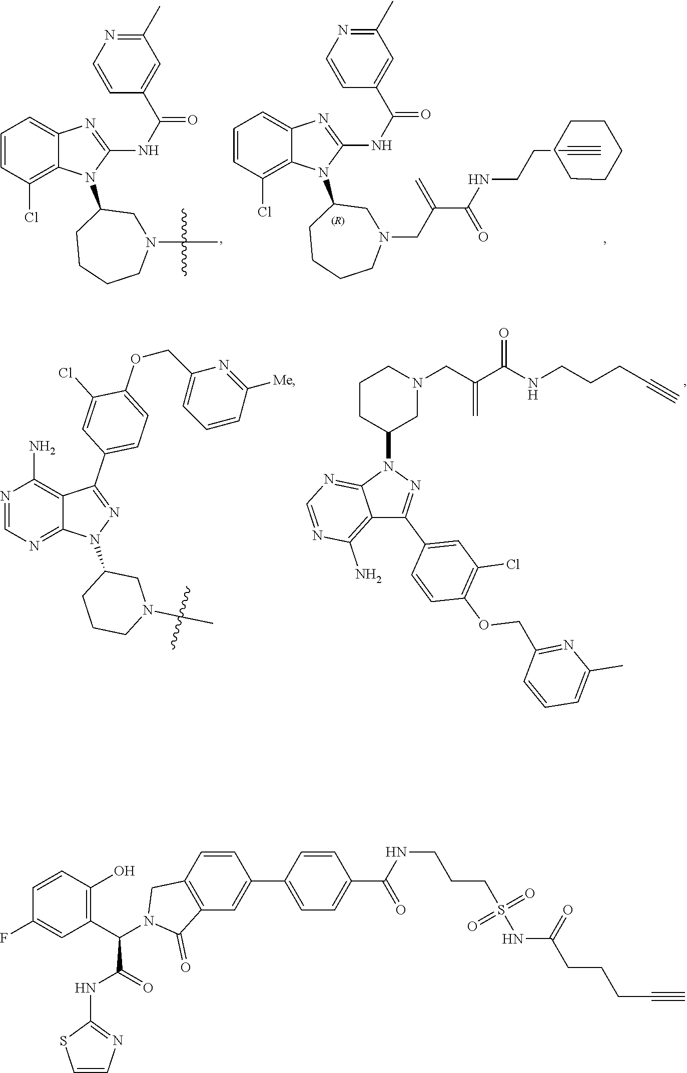

or a derivative thereof and the yne-group/hexagon represents the bonding point for the remaining chimeric small molecule.

In an example embodiment, the LIMK inhibitor is according to the formula:

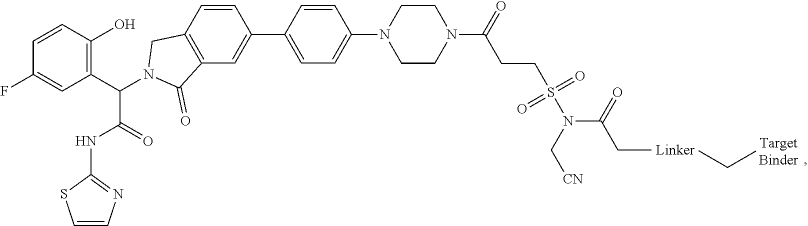

or a derivative thereof and the “Linker” and “Target Binder” represents the remaining chimeric small molecule.

### BCKDK Binding Moiety

In one example embodiment, the kinase binding moiety is a Branched chain alpha-ketoacid dehydrogenase kinase (BCKDK) binding moiety, also referred to as 3-methyl-2-oxoobutanoate-dehydrogenase kinase, binding moiety. In one example embodiment, the BCKDK binding moiety is an inhibitor or activator. BCKDK has been targeted to address conditions such as obesity, maple syrup urine disease and diabetes. In an embodiment, the binding moiety is ADR000362, which is according to the formula

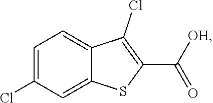 [SMILES: O=C(O)c1sc2cc(Cl)ccc2c1Cl]

or derivatives thereof.

In one embodiment, the allosteric inhibitor is the S-enantiomer of α-chlorophenylpropionate [(S)-CPP] according to the formula

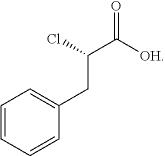 [SMILES: O=C(O)[C@@H](Cl)Cc1ccccc1]

See, Tso S C, Qi X, Gui W J, et al. Structure-based design and mechanisms of allosteric inhibitors for mitochondrial branched-chain α-ketoacid dehydrogenase kinase. Proc Natl Acad Sci USA. 2013; 110(24):9728-9733. doi:10.1073/pnas.1303220110, incorporated herein by reference, specifically, Table 1 inclusive of BCKDK inhibitor compounds and their IC₅₀ and K_{d} values.

In an embodiment, the BCKDK inhibitor is a benzothiophene carboxylate derivative. In an embodiment, the binding moiety is according to the formula

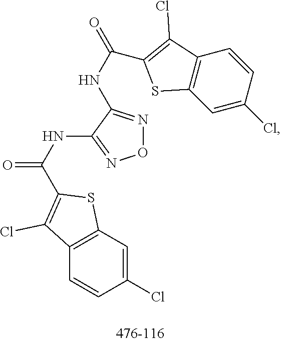 [SMILES: O=C(Nc1nonc1NC(=O)c1sc2cc(Cl)ccc2c1Cl)c1sc2cc(Cl)ccc2c1Cl]

and derivatives thereof. See, Tso et al., Benzothiophene carboxylate derivatives as novel allosteric inhibitors of branched-chain α-ketoacid dehydrogenase kinase. J Biol Chem. 2014 Jul. 25; 289(30):20583-93. doi: 10.1074/jbc.M114.569251.

### FGFR Binding Moiety

In one example embodiment, the protein binding moiety is an FGFR kinase binding moiety. In one example embodiment, the FGFR binding moiety is an inhibitor or activator. Fibroblast growth factor receptors (FGFRs) are a family of receptor tyrosine kinases expressed on the cell membrane and consists of four members: FGFR1 to FGFR4. All four FGFR members contain a large extracellular ligand-binding domain from the N- to the C-terminus that comprises three immunoglobulin (Ig)-like subunits (D1, D2 and D3) followed by a single transmembrane helix and an intracellular tyrosine kinase domain. The native ligand of FGFRs is fibroblast growth factors. FGFRs play a crucial role in both developmental and adult cells. See, e.g., Dai S., et al., “Fibroblast Growth Factor Receptors (FGFRs): Structures and Small Molecule Inhibitors.” Cells 2019, 8 (6), 614.

In an example embodiment, the FGFR inhibitor is SSR128129 according to the formula:

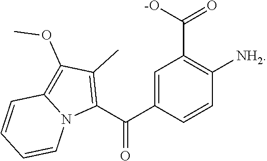

The SSR128129 properties comprise:

| | |
|---|---|
| Hydrogen bond acceptors | 4 |
| Hydrogen bond donors | 2 |
| Rotatable bonds | 4 |
| Topological polar surface area | 94.03 |
| Molecular weight | 324.11 |
| XLogP | 3.7 [symbol] |
| No. Lipinski's rules broken | 0 |

[symbol]

indicates data missing or illegible when filed

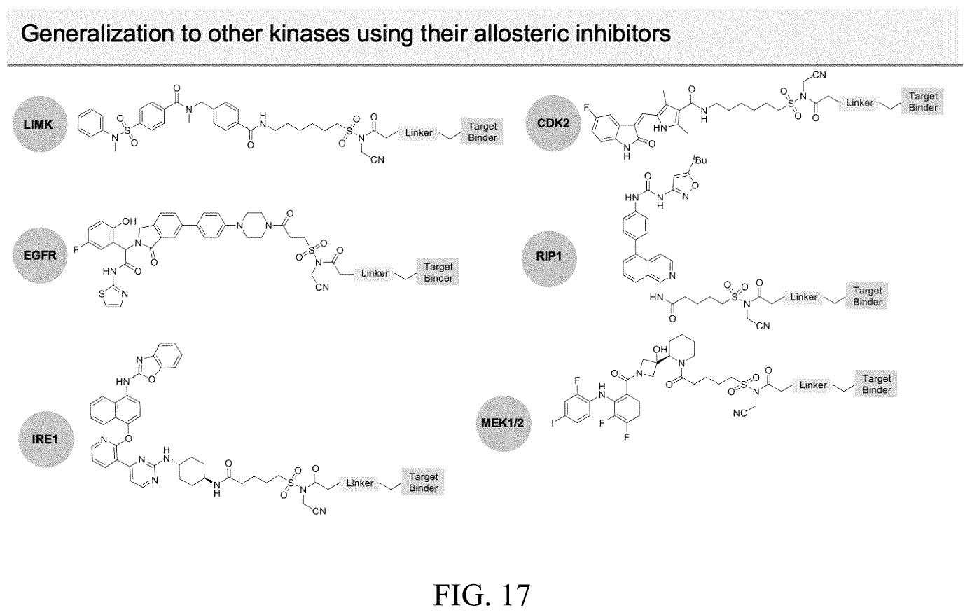

In an aspect, the SSR128129E is used as the sodium salt. SSR128129E is a negative allosteric modulator of the FGF receptor. The compound inhibits FGF1-induced ERK phosphorylation via FGFR2 with an IC50<100 nM. SSR128129E inhibits FGF ligand induction of receptor dimerization in an allosteric manner without affecting FGF binding, with interaction at Lys279, Thr320, Thr319, Cys 278, Trp290, Phe 276, Wal 274, Tyr 340, Ile329, Tyr328, Leu327, Leu312. See, Cancer Cell, 2013, 23, 477-488, incorporated by reference. For effects of SSR on cellular responses to different FGFRs, reference is made to Table 1 of Cancer Cell, 2013, 23, 4774-88, incorporated specifically herein by reference and showing SSR Concentration resulting in at least 50% inhibition at concentrations of between 10 nM and 100 nM. See also, generally, Herbert et al., Molecular Mechanism of SSR128129E, an Extracellularly Acting, Small-Molecule, Allosteric Inhibitor of FGF Receptor Signaling. Cancer Cell Jul. 11, 2016; doi: 10.1016/j.ccr.2013.02.018, incorporated by reference for chemical structure of the SSR128129E, predicted binding as detailed in FIG. 17 and effects of SSR on cellular responses to different FGFRs as provided in Table 1, each of which is specifically incorporated herein by reference.

In an example embodiment, the FGFR inhibitor is Pemigatinib, Infigratinib, Fisogatinib, or any derivatives thereof.

In an example embodiment, the FGFR binding moiety is according to the formula:

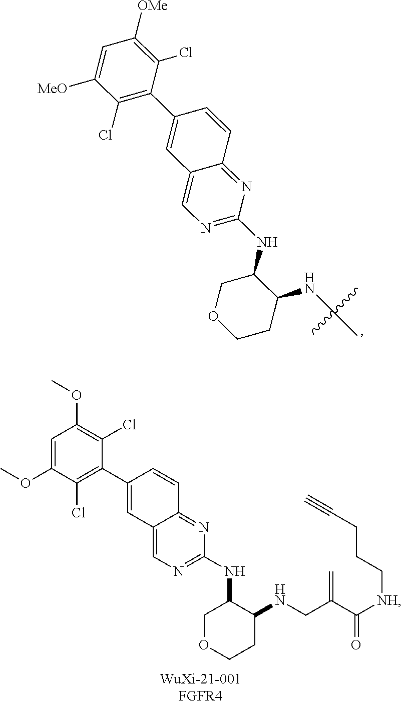 [SMILES: *N[C@H]1CCOC[C@H]1Nc1ncc2cc(-c3c(Cl)c(OC)cc(OC)c3Cl)ccc2n1 ; C#CCCCNC(=O)C(=C)CN[C@H]1CCOC[C@H]1Nc1ncc2cc(-c3c(Cl)c(OC)cc(OC)c3Cl)ccc2n1]

or a derivative thereof and the yne-group represents the bonding point for the remaining chimeric small molecule.

In an example embodiment, the kinase binding moiety is a pan-FGFR kinase binding moiety. In an example embodiment, the pan-FGFR binding moiety is an inhibitor or activator. In an example embodiment, the pan-FGFR inhibitor is Erdafitinib, Futibatinib, or any derivatives thereof.

In an example embodiment, the pan-FGFR binding moiety is according to the formula:

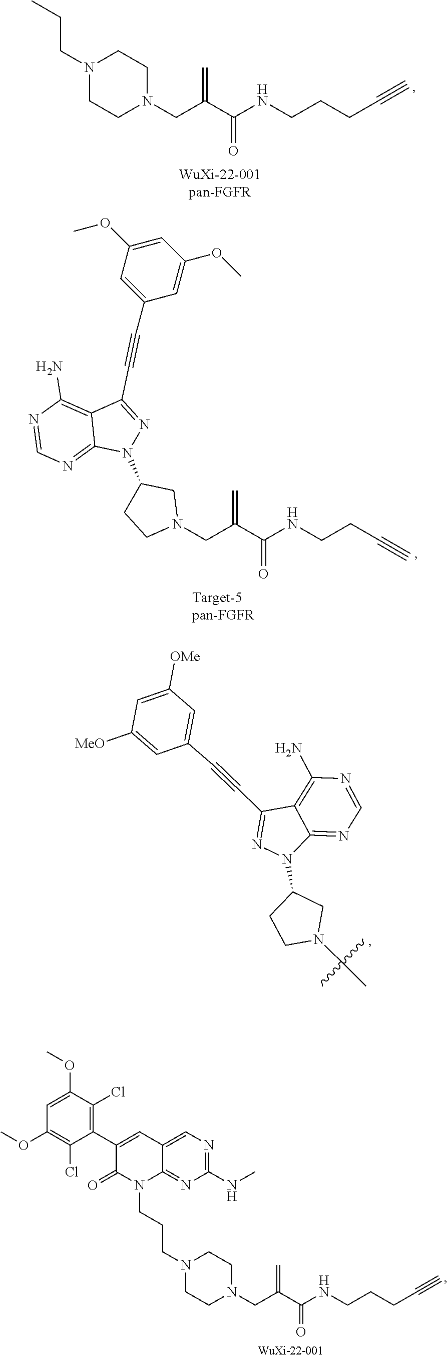

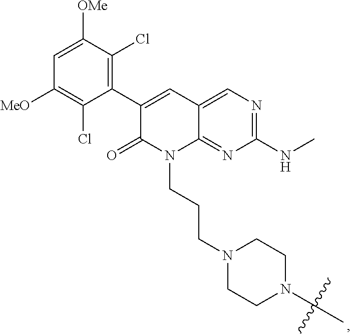 [SMILES: *N1CCN(CCCn2c(=O)c(-c3c(Cl)c(OC)cc(OC)c3Cl)cc3cnc(NC)nc32)CC1]

or a derivative thereof and the yne-group represents the bonding point for the remaining chimeric small molecule.

### HA-NGFR Binding Moiety

In one example embodiment, the protein binding moiety is an allosteric Tropomuosin receptor kinase A (TrkA), or a High affinity nerve growth factor receptors (HA-NGFR) kinase binding moiety. In one example embodiment, the TrkA or HA-NGFR binding moiety is an inhibitor or activator. High affinity nerve growth factor receptors (HA-NGFRs) are a family of receptor tyrosine nd regulates the proliferation, differentiation and survival of sympathetic and nervous neurons of the central and peripheral nervous systems. The native ligand of HA-NGFRs is nerve growth factors. The absence of the ligand resulting in lack of activation may promote cell death, making the survival of neurons dependent on trophic factors. See e.g. National Center for Biotechnology Information, 2021. PubChem Protein Summary for NCBI Protein P04629, High affinity nerve growth factor receptor.

In an embodiment, the pan Trk inhibitor is GZ389988, AR786 (allosteric selective TrkA inhibitor), ASP7962 (TrkA receptor antagonist), ONO-4474 (pan Trk inhibitor), or VM902A (allosteric TrkA selective inhibitor). Additional Trk inhibitors are described in Bailey et al, Tropomyosin receptor kinase inhibitors: an updated patent review for 2010-2016, doi: 10.1080/13543776.2017.1297797, and Bailey et al., (2020) Tropomyosin receptor kinase inhibitors: an updated patent review for 2016-2019, Expert Opinion on Therapeutic Patents, 30:5, 325-339, DOI: 10.1080/13543776.2020; both incorporated herein by reference in their entirety.

In an example embodiment, the HA-NGFR is VM-902A or a related compound or analog thereof. In an aspect, the compound can be

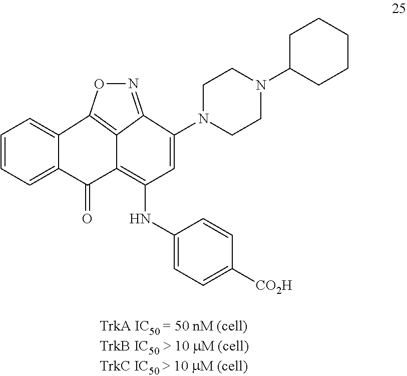 [SMILES: O=C(O)c1ccc(Nc2cc(N3CCN(C4CCCCC4)CC3)c3noc4c3c2C(=O)c2ccccc2-4)cc1]

or an analog thereof.

### IkappaB Binding Moiety

In one embodiment, the protein binding moiety is an IkappaB kinase binding moiety. In one example embodiment, the IkappaB binding moiety is an inhibitor or activator. In an aspect, the IKappaB kinase binding moiety inhibits one or both subunits IKK-alpha and IKK-beta of IkappaB kinase. In one embodiment, the binding moiety is a selective allosteric inhibitor BMS-345541 and is according to the formula:

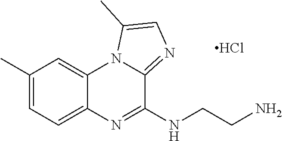 [SMILES: Cc1ccc2nc(NCCN)c3ncc(C)n3c2c1 ; Cl]

with the following properties

| | |
|---|---|
| Hydrogen bond acceptors | 4 |
| Hydrogen bond donors | 2 |
| Rotatable bonds | 3 |
| Topological polar surface area | 68.24 |
| Molecular weight | 255.15 |
| XLogP | 2.15 |
| No. Lipinski's rules broken | 0. |

BMS-345541 has been shown to block NF-κB dependent transcription in mice, and is active against LPS-induced NF-kB activation in mice. In an aspect, the negative allosteric modulator BMS-345541 has a pK_{d} oof 6.9, a pIC₅₀ of 6.5 as an inhibitor of nuclear factor kappaB kinase subunit beta, and a pIC₅₀ of 5.4 of component of inhibitor of nuclear factor kappa B kinase complex.

### CDK Binding Moiety

In one example embodiment, the protein binding moiety is an CDK kinase binding moiety. In one example embodiment, the protein binding moiety is a CDK inhibitor or activator. The cyclin-dependent kinases (CDKs) are characterized by needing a separate subunit, cyclin, that provides domains for enzymatic activity. CDK controls cell division and modulates transcription. The CKD family is divided into three cell-cycle-related subfamilies: CDK1, CDK 2, and CDK 3; CDK4 and CDK6; and CDK5, and CDK14-CDK18 as well as five transcriptional subfamilies: CDK7; CDK8 and CDK 19; CDK9; CDK10 and CDK 11; CDK12 and CDK13; and CDK20. In one example embodiment, the CDK inhibitor comprises Palbociclib, Ribociclib, Abemaciclib, or any derivatives thereof. In an example embodiment, the CDK8 inhibitor is compound 5 with the formula:

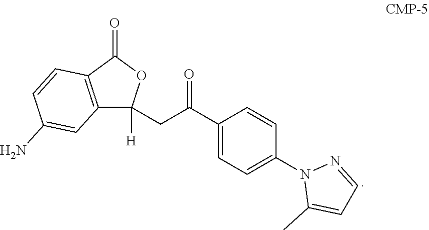 [SMILES: Cc1ccnn1-c1ccc(C(=O)CC2OC(=O)c3ccc(N)cc32)cc1]

In an example embodiment, the CDK2 inhibitor is a flavopiridol analog. In an example. Embodiment, the CDK2 inhibitor is 8-amidoflavone, 8-sulfonamidoflavone, 8-amido-7-hydroxyflavone, or heterocyclic analogues of flavopiridol. See, Ahn et al., Design, synthesis, and antiproliferative and CDK2-cyclin a inhibitory activity of novel flavopiridol analogues, Bioorganic & Medicinal Chemistry, Volume 15, Issue 2, 2007, Pages 702-713, doi: 10.1016/j.bmc.2006.10.063, incorporated herein by reference. In one embodiment, the compound is selected from the 8 aminoflavopiridol analogues of Table 1 of Ahn, and may be selected based on antiproliferative and inhibitory activities of Table 1, incorporated specifically herein by reference. Modifications to the molecules of Ahn may be made based in part on the desired interactions between the analog and CDK, with exemplary modifications made based on FIG. 2A-2B.

In an example embodiment, the CDK inhibitor is Alvocidib, an inhibitor which causes cell-cycle arrest and is in Phase 2 clinical evaluation for anti-cancer potential, according to the formula:

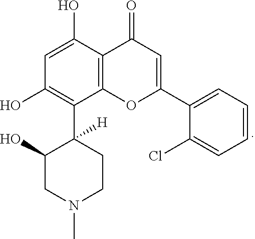 [SMILES: CN1CC[C@H](c2c(O)cc(O)c3c(=O)cc(-c4ccccc4Cl)oc23)[C@H](O)C1]

In an embodiment, Alvocidib is utilized as a CDK2 and/or CDK4 kinase binding moiety, with a CDK4 pKᵢ of 7.2 and a CDK2 pIC₅₀ of 6.4-7.0, and with the following properties:

| | |
|---|---|
| Hydrogen bond acceptors | 3 |
| Hydrogen bond donors | 3 |
| Rotatable bonds | 2 |
| Topological polar surface area | 84.14 |
| Molecular weight | 401.1 |
| XLogP | [symbol] |
| No. Lipinski's rules broken | 0 |

[symbol]

indicates data missing or illegible when filed

In an aspect, properties can be optimized for use in the chimeric small molecules based on sites for modification which may be identified and optimized in accordance with the formula, and as discussed in Bioorg. Med. Chem. 2007, 15, 702-713:

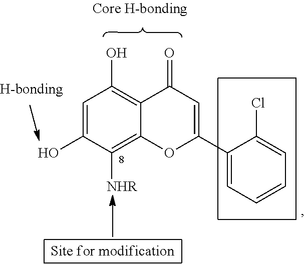

wherein R can be any cyclic hydrocarbon; an unsaturated cyclic hydrocarbon; a heterocycle; one or more fused rings comprising any combination of any previously mentioned rings optionally substituted at one or more positions alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, cyclic hydrocarbon, an unsaturated cyclic hydrocarbon, a heterocycle; one or more fused rings comprising any combination of any previously mentioned rings, preferably a piperideine, pyrollidine, thiane, or morpholine ring which may be further substituted at any position on the ring. Particular 8 aminoflavopiridol analogues are as detailed in table 1 of Bioorg. Med. Chem. 2007, 15, 702-713, depicted below:

TABLE 1
Antiproliferative and CDK2-Cyclin A inhibitory activities
of S-amino [symbol] vopiridol anal [symbol] gues

| | ID- [symbol] IC₅₀ | MC [symbol] -7 [symbol] IC [symbol] | CDK2-Cyclin A |
|---|---|---|---|
| Compound | (μM) | (μM) | (μM) |
| Flavopirid [symbol] l ( [symbol] ) | 0.0070 | 0.026 | 1.5 |
| 17a | 1 [symbol] | [symbol] .1 | 417 |
| 17b | [symbol] | 4.6 | 417 |
| 17c | [symbol] 3 | 1 [symbol] | 217 |
| 17d | 16 | 10 | N.D. |
| 17e | [symbol] | [symbol] | 383 |
| 19a | N.D. | 8.5 | 91 |
| 19b | N.D. | [symbol] 3 | [symbol] |
| 19c | N.D. | 9.7 | [symbol] 0 |
| 19d | N.D. | 13 | [symbol] 4 |
| 20a | 9. [symbol] | 20 | 417 |
| 20b | 5.7 | 17 | 417 |
| 20c | 7.9 | 20 | 417 |
| 21a | 104 | 5.5 | N.D. |
| 21b | 12 | 4.5 | 417 |
| 21c | 17 | 2. [symbol] | 417 |
| 21d | 1 [symbol] | 2.8 | N.D. |
| 22a | [symbol] 8 | [symbol] | 417 |
| 22b | [symbol] | 7. [symbol] | 417 |
| 22c | [symbol] | 4.0 | 417 |
| 22d | [symbol] 2 | 20 | N.D. |
| 23 | 18 | 30 | 417 |
| 24a | 16 | 25 | 219 |
| 24b | 9.5 | 16 | 178 |
| 24c | [symbol] 4 | 17 | [symbol] 4. |

[symbol]

indicates data missing or illegible when filed

In an example embodiment, The CDK inhibitor is according to the formula:

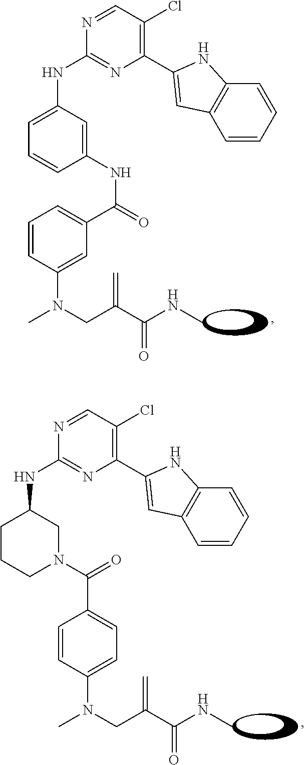 [SMILES: C=C(CN(C)c1cccc(C(=O)Nc2cccc(Nc3ncc(Cl)c(-c4cc5ccccc5[nH]4)n3)c2)c1)C(=O)NC ; C=C(CN(C)c1ccc(C(=O)N2CCC[C@@H](Nc3ncc(Cl)c(-c4cc5ccccc5[nH]4)n3)C2)cc1)C(=O)NC]

or a derivative thereof and the oval shape represents the remaining chimeric small molecule.

In an example embodiment, the CDK2 inhibitor is according to the formula:

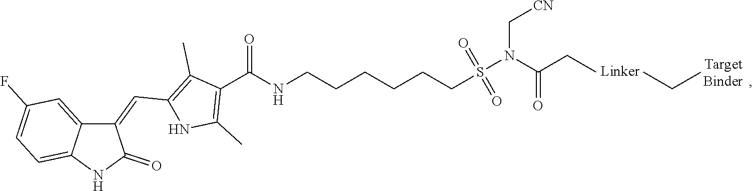

or a derivative thereof and the “Linker” and “Target Binder” represents the remaining chimeric small molecule.

### PI3K Binding Moiety

In one example embodiment, the protein binding moiety is an PI3K kinase binding moiety. In one example embodiment, the protein binding moiety is a PI3K inhibitor or activator. The phosphoinositide 3-kinase (PI3K) is a superfamily of lipid kinases central to human cancer, diabetes, and aging. There are three different PI3K classes (I, II and III), as well as for the different isoforms (e.g. Class I has 4 isoforms: α, β, γ, δ) and within each class there are distinct roles for each of the PI3Ks. Class I has been implicated in many cancers particularly those with pathogenic mutations. PI3K acts downstream to many growth factors and acts upstream to AKT and mTOR. (Kannaiyan et al. Expert Rev Anticancer Ther. 2018; 18(12): 1249-1270)

In one example embodiment, the PI3K inhibitor is Idelalisib with the formula:

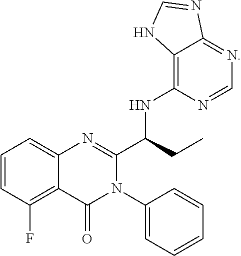 [SMILES: CC[C@H](Nc1ncnc2nc[nH]c12)c1nc2cccc(F)c2c(=O)n1-c1ccccc1]

Idelalisib is a small molecule inhibitor of the delta isoform of PI3K. In an example embodiment, the PI3K inhibitor is PIK-108 according to the formula:

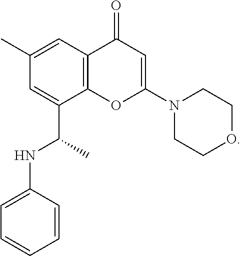 [SMILES: Cc1cc([C@H](C)Nc2ccccc2)c2oc(N3CCOCC3)cc(=O)c2c1]

PIK-108 is an allosteric inhibitor of the lipid modifying kinases, phosphatidylinositol-4,5-bisphosphate 3-kinase catalytic subunits β and 8 (PI3 KB/8). The compound binds at an allosteric site close to the mutation hotspot of H1047R in the mouse PI3Kα C-lobe, in addition to binding at the ATP-binding pocket. See e.g. Certal, V., et al. “Discovery and Optimization of New Benzimidazole- and Benzoxazole-Pyrimidone Selective PI3 KB Inhibitors for the Treatment of Phosphatase and TENsin Homologue (PTEN)-Deficient Cancers.” J. Med. Chem. 2012, 55 (10), 4788-4805, herein incorporated by reference in its entirety with specific mention of Table 2 and 3 and the Biochemical and Cellular Activity of Pyrimidone Benzimidazoles and their substitutions.

### VEGFR Binding Moiety

In one example embodiment, the protein binding moiety is a VEGFR binding moiety. In one example embodiment, the VEGFR binding moiety is an inhibitor or activator. Vascular endothelial growth factors (VEGFs) are a family of polypeptides with conserved receptor-binding domain comprising of a disulfide-knot structure. There are two VEGFs, VEGF-A and VEGF-B, that bind to VEGFR which are receptor tyrosine kinases located on vascular endothelial cells. In one example embodiment, the kinase binding moiety is a VEGFR inhibitor. In an example embodiment, the VEGFR inhibitor is Sorafenib, Sunitinib, Pazopanib, Axitinib, Cabozantinib, Lenvatinib, Vandetanib, or Regorafenib.

### BRAF Binding Moiety

In one example embodiment, the kinase binding moiety is a BRAF binding moiety. In one example embodiment, the binding moiety is a BRAF inhibitor or activator. BRAF is a member of the Rapidly Accelerated Fibrosarcoma family of serine/threonine kinases and is frequently activated in patients with cancer through genetic aberrations. BRAF has three conserved regions: conserved region 1 (CR1) is a Ras-GTP-binding self-regulatory domain; conserved region 2 (CR2) is a serine-rich region that functions as a hinge on the molecule; and conserved region 3 (CR3) is a catalytic protein kinase domain. In one example embodiment, the kinase binding moiety is a BRAF inhibitor. In one embodiment, the BRAF inhibitor comprises Vemurafenib or Dabrafenib.

### MEK Binding Moiety

In one example embodiment, the protein binding moiety is a MEK binding moiety. In one example embodiment, the MEK binding moiety is an inhibitor or activator. MEK is a kinase enzyme that phosphorylates mitogen activated protein kinases (MAPK). Seven MEK subtypes have been identified, all mediate cellular responses to different growth signals. In one example embodiment, the kinase binding moiety is a MEK inhibitor. In one embodiment, the binding moiety is a Type-3 kinase inhibitor. In one embodiment, the MEK inhibitor comprises Trametinib according to the formula:

Trametinib has been used for the adjuvant treatment of patients with BRAF V600E or V600K mutated melanoma inhibiting MAP2K1 and MAP2K2 (aka MEK1 and 2) in the p42/p44 MAPK pathway. Absorption/distribution of an oral dose of trametinib tablet is 72%. Trametinib is 97.4% bound to human plasma proteins, which can be utilized when determining dosage for small molecules detailed herein. See, Gilmartin, et al., GSK1120212 (JTP-74057) Is An Inhibitor of MEK Activity and Activation with Favorable Pharmacokinetic Properties for Sustained In Vivo Pathway Inhibition. Clin Cancer Res. 2011 Mar. 1; 17(5):989-1000. doi: 10.1158/1078-0432.CCR-10-2200. Epub 2011 Jan. 18. Trametinib has a MAPK1 inhibition pIC₅₀ of 9/0-9.1 and a MAPK2 pIC50 inhibition of 8.7. Trametinib was shown to have sustained suppression of p-ERK 1/2 for more than 24 hours, with high potency, selectivity and long circulating half-life.

In one example embodiment, the MEK inhibitor comprises Cobimetinib, an allosteric inhibitor of MEK serine/threonine protein kinases, with a selectivity for MEK 1 and MEK2. Cobimetinib selectively inhibits the activity of the MEK serine threonine protein kinase and is according to the formula:

with the following properties:

| | |
|---|---|
| Hydrogen bond acceptors | 5 |
| Hydrogen bond donors | 3 |
| Rotatable bonds | 5 |
| Topological polar surface area | 64.6 |
| Molecular weight | [symbol] 31.0 [symbol] |
| XLogP | 4.82 |
| No. Lipinski's rules broken | 0. |

[symbol]

indicates data missing or illegible when filed

Additional in vitro activity of cobimetinib and related analogs was explored in Rice et al., “Novel Carboxamide-Based Allosteric MEK Inhibitors: Discovery and Optimization Efforts toward XL518 (GDC-0973)” ACS Med. Chem. Lett. 2012, 3, 5, 416-421, incorporated herein by reference in particular, at Tables 1 and 3.

In an example embodiment, the RIP1 inhibitor is according to the formula:

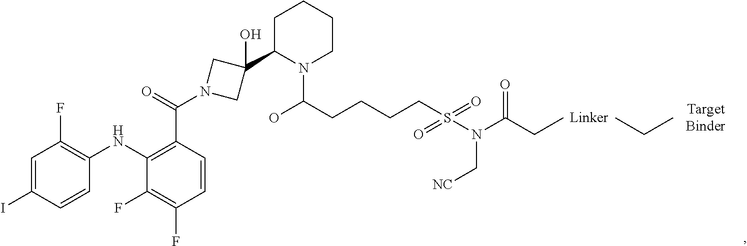

or a derivative thereof and the “Linker” and “Target Binder” represents the remaining chimeric small molecule.

In an example embodiment, the MEK inhibitor comprises Pimasertib according to the formula:

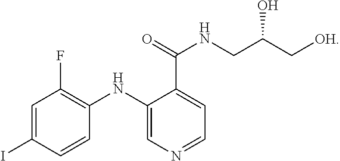 [SMILES: O=C(NC[C@H](O)CO)c1ccncc1Nc1ccc(I)cc1F]

Pimasertib is an orally bioavailable small-molecule inhibitor of the mitogen-activated protein kinases MEK1 and MEK2 (MEK1/2) with potential antineoplastic activity. It binds to an allosteric site, distinct from the ATP binding site and as such prevents activation rather than inhibiting catalysis. Pimasertib (AS703026) is cytotoxic against CD138-purified multiple myeloma (MM) cells from patients with relapsed and refractory MM, with IC50 values ranging from 2-200 nM. MEK1/2 (MAP2K1/K2) are dual-specificity threonine/tyrosine kinases that play key roles in the activation of the RAS/RAF/MEK/ERK pathway and are often upregulated in a variety of tumor cell types. Selectively binds to and inhibits the activity of MEK1/2, preventing the activation of MEK 1/2-dependent effector proteins and transcription factors, which may result in the inhibition of growth factor-mediated cell signaling and tumor cell proliferation. See, Yoon J, Koo K H, Choi K Y. MEK1/2 inhibitors AS703026 and AZD6244 may be potential therapies for KRAS mutated colorectal cancer that is resistant to EGFR monoclonal antibody therapy. Cancer Res. 2011 Jan. 15; 71(2):445-53. doi: 10.1158/0008-5472.

In an example embodiment, the MEK inhibitor comprises CI-1040 according to the formula:

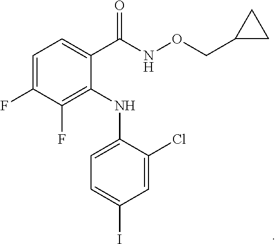 [SMILES: O=C(NOCC1CC1)c1ccc(F)c(F)c1Nc1ccc(I)cc1Cl]

In an example embodiment, the MEK1 and MEK2 inhibitor is Selumetinib (AZD6244, ARRY-142886) according to the formula:

and with the following properties:

| | |
|---|---|
| Hydrogen bond acceptors | 4 |
| Hydrogen bond donors | 3 |
| Rotatable bonds | [symbol] |
| Topological polar surface area | 88.41 |
| Molecular weight | 4 [symbol] |
| XLogP | 3. [symbol] |
| No. Lipinski's rules broken [symbol] | 0 |

[symbol]

indicates data missing or illegible when filed

Selumietinib is an orally bioavailable non-ATP competitive inhibitor that is highly specific for MEK1/2. It is a negative allosteric modulator of MEK1 with a pIC50 of 7.8-7.9. Sensitivity to selmuetinib in a panel of NSCLC and CRC cell lines showed sensitivity to particular mutations of KRAS in GEO cells with amino acid change p.G12A, SW480 cells with amino acid change G12V, SW620 cells with amino acid change p.G12V, and in HCT116 cells with amino acid change G13D and PIK3CA amino acid change p.H1047R, and in H1299 cells with NRAS amino acid change p.Q61K.

In an example embodiment, the allosteric MEK inhibitor is a 3,4-difluoro-2-(2-halo-4-iodo-phenylamino)-N-2-hydroxy-ethoxy)-benzamide according to the formula

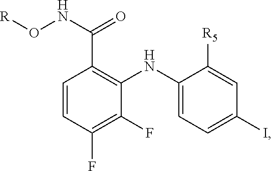

wherein R and R5 are selected from the table below

| | | | C26 | Sol |
|---|---|---|---|---|
| | | | IC₅₀ | (pH 6.5) |
| Compound | R | R [symbol] | (nM) | (μg/mL) |
| 24 (C [symbol] -1040) | —CH₂ [symbol] Pr | Cl | 35 | <1 |
| 29 | —CH₂ [symbol] Pr | F | 1.0 | <1 |
| 30 | —CH₂CH₂OH | Cl | 3.5 | <1 |
| 31 | —CH₂CH₂OH | F | 0.07 | 5 |
| 32 | (±)—CH₂CHOH(CH₂OH) | Cl | 19 | — |
| 33 | (±)—CH₂CHOH(CH₂OH) | F | 0.48 | 147 |
| 34 (PD 0325901) | R-(−)—CH₂CHOH(CH₂OH) | F | 0.33 | 190 |
| 35 | S-(+)—CH₂CHOH(CH₂OH) | F | 0.82 | 255, |

[symbol]

indicates data missing or illegible when filed

as described in Hartung et al., Optimization of allosteric MEK inhibitors, Part 1: Venturing into underexplored SAR territories, Bioorganic and Medicinal Chemistry Letters 23 (2013) 2384-2390, incorporated herein by reference.

In one example embodiment, the MEK inhibitor is Mirdametinib (PD 0325901) a selective and non-ATP-competitive MEK inhibitor that has been explored in advanced KRAS mutant colorectal cancer, non-small-cell lung cancer, melanoma, colonic neoplasms and breast cancer, and is according to the formula:

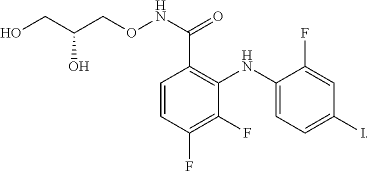 [SMILES: O=C(NOC[C@H](O)CO)c1ccc(F)c(F)c1Nc1ccc(I)cc1F]

Mirdametinib has a EK1 inhibition pIC₅₀ value of 8.1 and has the following properties:

| | |
|---|---|
| Hydrogen bond acceptors | 4 |
| Hydrogen bond donors | 4 |
| Rotatable bonds | 8 |
| Topological polar surface area | [symbol] 0.82 |
| Molecular weight | 482 |
| XLogP | 3.4 |
| No. Lipinski's rules broken | 0 |

[symbol]

indicates data missing or illegible when filed

In one example embodiment, the MEK binding moiety comprises allosteric inhibitor refametinib:

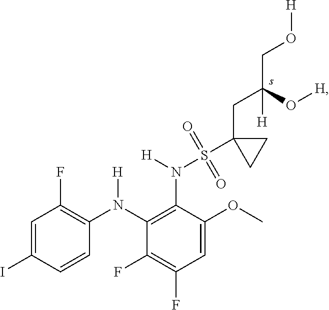

or an analog thereof, for example,

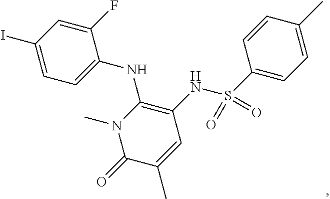 [SMILES: Cc1ccc(S(=O)(=O)Nc2cc(C)c(=O)n(C)c2Nc2ccc(I)cc2F)cc1]

or a derivative thereof.

In an example embodiment, the MEK inhibitor is Binimetinib according to the formula; and has the following properties:

| | |
|---|---|
| Hydrogen bond acceptors | 4 |
| Hydrogen bond donors | [symbol] |
| Rotatable bonds | 7 |
| Topological polar surface area | 88.41 |
| Molecular weight | 440.03 |
| XLogP | 3.2 [symbol] |
| No. Lipinski's rules broken | 0 |

[symbol]

indicates data missing or illegible when filed

Binimetinib has received FDA approval as a treatment for advanced BRAF-mutant melanoma in conjunction with the BRAF mutant kinase inhibitor encorafenib. See, Dummer et al., Encorafenib plus binimetinib versus vemurafenib or encorafenib in patients with BRAF-mutant melanoma (COLUMBUS): a multicenter, open-label, randomized phase 3 trial. Lancet Oncol. 2018 May; 19(5):603-615. doi: 10.1016/S1470-2045(18)30142-6.

Additional exploration in other solid tumor types, neuroblastoma, and hematological cancers are being explored. See, e.g. Woodfield S E, Zhang L, Scorsone K A, Liu Y, Zage P E. Binimetinib inhibits MEK and is effective against neuroblastoma tumor cells with low NF1 expression. BMC Cancer. 2016 Mar. 1; 16:172. doi: 10.1186/s12885-016-2199-z. Binimetinib is a negative allosteric modulator with a MEK1 and a MEK2 pIC50 of 7.9.

### AKT Binding Moiety

In one example embodiment, the protein binding moiety is an AKT binding moiety. In one example embodiment, the AKT binding moiety is an inhibitor or activator. RAC-alpha serine/threonine-protein kinase (AKT) in humans has three isozymes (AKT1, 2, and 3, also known as PKB-α, -β and -γ). Each isozyme contains an amino (N)-terminal PH domain, inter-domain linker, kinase domain and 21-residue carboxy (C)-terminal hydrophobic motif. In an example embodiment, the ATK inhibitor is Borussertib with the formula:

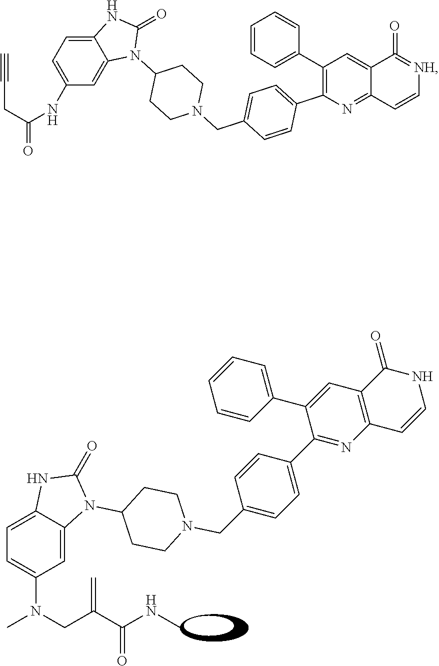

or a derivative thereof and the oval shape represents the remaining chimeric small molecule.

In an example embodiment, the kinase inhibitor is MK-2206 with the formula:

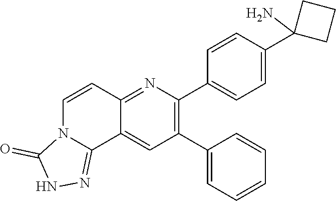 [SMILES: NC1(c2ccc(-c3nc4ccn5c(=O)[nH]nc5c4cc3-c3ccccc3)cc2)CCC1]

with the following properties:

| | |
|---|---|
| Hydrogen bond acceptors | 4 |
| Hydrogen bond donors | 2 |
| Rotatable bonds | 3 |
| Topological polar surface area | 89.07 |
| Molecular weight | 407.17 |
| XLogP | 5.13 |
| No. Lipinski's rules broken | 1. |

MK-2206 is an orally bioavailable allosteric inhibitor of the serine/threonine protein kinase AKT (protein kinase B) with potential antineoplastic activity. MK-2206 has pIC50 values of 8.3, 7.9, and 7.2 for AKT1, 2, and 3 respectively. MK-2206 is able to enhance the antitumor efficacy of standard chemotherapeutic agents or molecular targeted drugs in vitro and in vivo. As of 2018, ClinicalTrilas.gov had 50 registered MK-2206 trials. Many have been withdrawn, terminated or completed. The ring fused to the pyridine may be modified to mono-, bi-, tricyclic linear fused rings, or angular tricycles. The pyridine may be modified to a pyrazine. The moiety of substituted benzene may also be modified. The strained cyclobutene may be substituted for any substituent known in the art. Furthermore, the hydrogens on the amine in the moiety may be substituted for any substituent known in the art. For further design guidance see Kettle, J. G., et al. “Diverse Heterocyclic Scaffolds as Allosteric Inhibitors of AKT.” J. Med. Chem. 2012, 55 (3), 1261-1273, herein incorporated by reference in its entirety.

In one example embodiment, the AKT inhibitor is any inhibitor from International Patent Application WO2008070016A2, herein incorporated by reference in its entirety, and any derivative thereof. In addition, see e.g. Wu, W.-I., et al. “Crystal Structure of Human AKT1 with an Allosteric Inhibitor Reveals a New Mode of Kinase Inhibition.” PLOS ONE 2010, 5 (9), e12913, herein incorporated by reference in its entirety. In an embodiment, inhibitors of AKT or derivatives thereof can be according to:

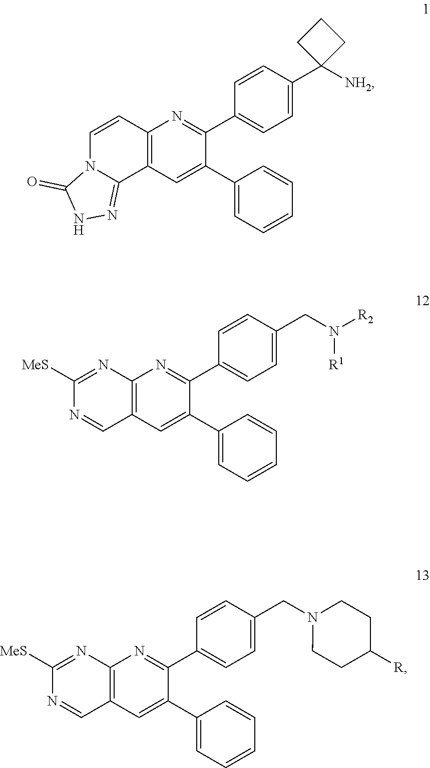

wherein R1=H and R2=

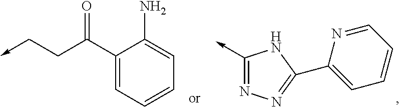

where any N in the ring can be substituted with C, N, O. S, B or P;
or according to

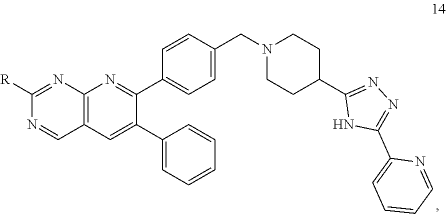

wherein R═NHMe. See, Bioorg. Med. Chem. Let. 2008, 18, 4191-4194; doi:10.1371/journal.pone.0012913, incorporated herein by reference.
Optimization of allosteric inhibition of AKT can be performed based on the following guidance:

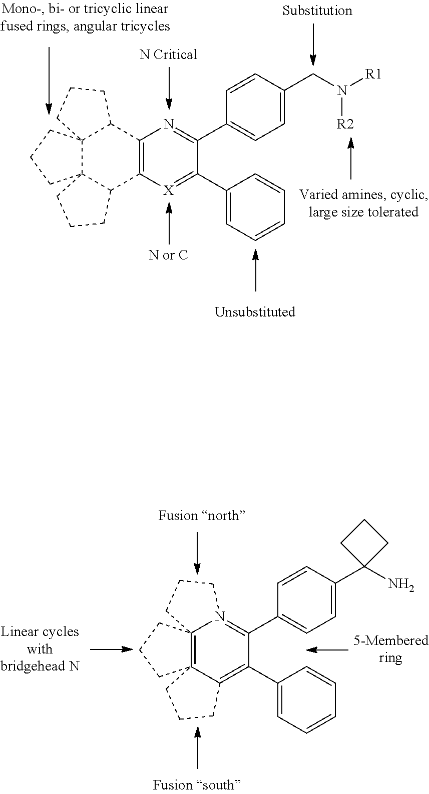

Strategy for combining potency and reducing hERG affinity for AKT binders can be based in whole or in part on the following:

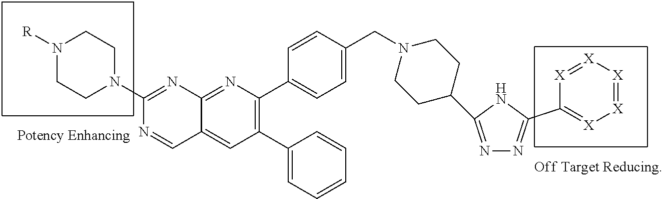

In an example embodiment, the kinase inhibitor is AKT Inhibitor VIII, also known as compound 16 h, with the formula:

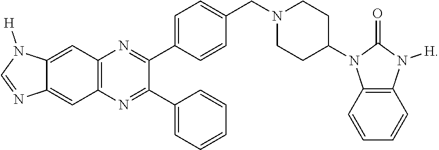

AKT Inhibitor VII is a cell-permeable quinoxaline compound that has been shown to potently, selectively, allosterically, and reversibly inhibit AKT (protein kinase B), with selectivity for AKT1 and 2 over AKT3. The pIC50 values of AKT Inhibitor VIII is 7.2, 6.7, and 5.7 for AKT1, 2, and 3 respectively. See Lindsley, C. W., et al. “Allosteric Akt (PKB) Inhibitors: Discovery and SAR of Isozyme Selective Inhibitors.” Bioorganic & Medicinal Chemistry Letters 2005, 15 (3), 761-764, herein incorporated by reference in its entirety with specific mention of Table 1 and Table 2, reproduced below:

TABLE 1
Structures and activities for pyrazinones 13/14

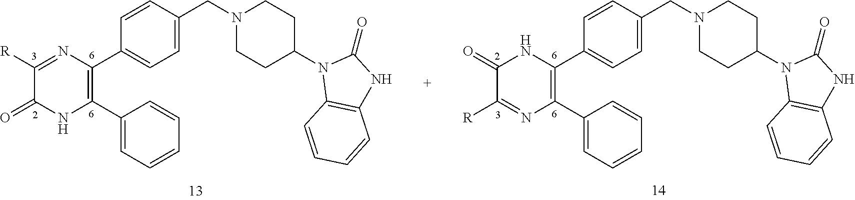

| | | Akt1 IC₅₀ | Akt2 IC₅₀ | Akt3 IC₅₀ |
|---|---|---|---|---|
| Compd | R | (nM) [symbol] | (nM) [symbol] | (nM) [symbol] |
| 13a | H | 3029 | 15,700 | >50,000 |
| 14a | | 1500 | >50,000 | >50,000 |
| 13b | CH₃ | 760 | 24,000 | >50,000 |
| 14b 13c 14c | 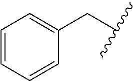 [SMILES: *Cc1ccccc1] | 1003 17,000 >50,000 | 1179 >50,000 1755 | 33,100 >50,000 3973 |
| 13d 14d | 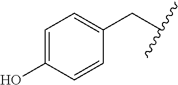 [SMILES: *Cc1ccc(O)cc1] | 23,670 >50,000 | 45,270 3407 | >50,000 >50,000 |
| 13e 14e | 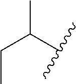 [SMILES: *C(C)CC] | 17,000 >50,000 | >50,000 4517 | >50,000 >50,000 |
| 13f 14f | 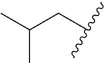 [SMILES: *CC(C)C] | >50,000 21,200 | 18,000 325 | >50,000 21,870 |

[symbol]

indicates data missing or illegible when filed

TABLE 2
Structures and activities for quinoxalines 16

| | |
|---|---|
| | 16 |
| 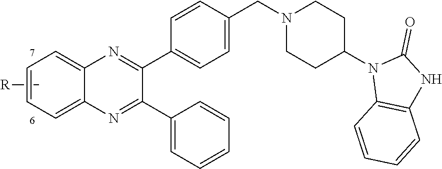 | |

| | | Akt1 IC₅₀ | Akt2 IC₅₀ | Akt3 IC₅₀ |
|---|---|---|---|---|
| Compd | R | (nM) [symbol] | (nM) [symbol] | (nM) [symbol] |
| 16a | 6-COOH | 240 | 281 | >50,000 |
| 16b | 7-COOH | 165 | 388 | 3200 |
| 16c | 6-(2H-tetrazole) | 63 | 65 | 1228 |
| 16d | 7-(2H-tetrazole) | 20 | 144 | 1613 |
| 16e | 6-(2-Me-tetrazole) | 1089 | 1877 | >50,000 |
| 16f | 7-(2-Me-tetrazole) | 55 | 332 | >50,000 |
| 16g | 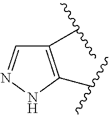 [SMILES: *c1cn[nH]c1*] | 85 | 300 | 2400 |
| 16h | 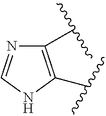 [SMILES: *c1nc[nH]c1*] | 58 | 210 | 2119. |

[symbol]

indicates data missing or illegible when filed

In an example embodiment, the kinase inhibitor is miransertib, also known as ARQ-092, according to the formula:

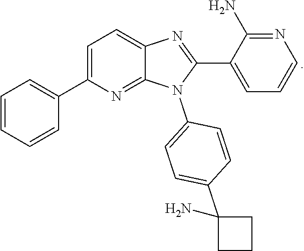 [SMILES: Nc1ncccc1-c1nc2ccc(-c3ccccc3)nc2n1-c1ccc(C2(N)CCC2)cc1]

Miransertib is an orally active, selective, and potent allosteric AKT inhibitor. Miransertib has pIC50 values of 8.3, 8.4, and 7.8 for AKT1, 2, and 3. Miransertib has progressed to Phase 1 and 2 development in solid and liquid tumors. See e.g. “Discovery of 3-(3-(4-(1-Aminocyclobutyl)Phenyl)-5-Phenyl-3H-Imidazo[4,5-b]Pyridin-2-Yl)Pyridin-2-Amine (ARQ 092): An Orally Bioavailable, Selective, and Potent Allosteric AKT Inhibitor.” J. Med. Chem. 2016, 59 (13), 6455-6469, herein incorporated by reference in its entirety with specific mention of Tables: 1, 2, 3, 4, 6, and 9, with Tables 2 and 4 reproduced below:

TABLE 2
Structure-Activity Relationship for Substitution on the Pyridine Ring^{a}

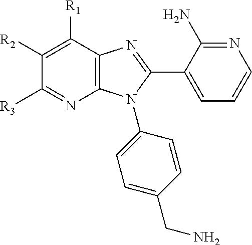

| | | | | IC₅₀ (μM)^{b} | | |
|---|---|---|---|---|---|---|
| cmpd | R₁ | R₂ | R₃ | AKT1 | AKT2 | AKT3 |
| 6 | H | H | H | 3.7 | 9.3 | >100 |
| 7a | Me | H | H | 0.5 | NT | NT |
| 7b | H | Me | H | 0.37 | 5.3 | >10 |
| 7c | H | Me | Me | >1 | NT | NT |
| 7d | H | H | Cl | 0.27 | 1.9 | 2.6 |
| 7e | H | Br | H | 0.32 | NT | NT |
| 9a | H | H | Ph | 0.0037 | 0.033 | 0.43 |
| 9b | H | H | 3-NHAc-Ph | 0.013 | 0.024 | 0.086 |
| 9c | H | H | 4-NHAc-Ph | 0.072 | 0.079 | 0.70 |
| 9d | H | H | 1H-pyrazol-4-yl | 0.017 | 0.014 | 0.065 |
| 9e | H | pyridin-3-yl | H | 0.036 | 3.5 | >10 |

^{a}See Experimental Section for assay details.
NT means not tested.
^{b}Assay conducted with unphosphorylated enzymes.

TABLE 4
Structure-Activity Relationship of S-Substituted Analogs

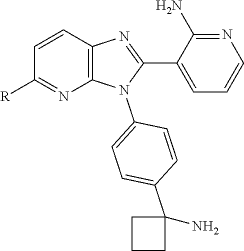

| | | IC₅₀ (μM) for unphosphorylated and | | | | | |
|---|---|---|---|---|---|---|---|
| | | phosphorylated forms of AKT isoforms [symbol] | | | | | |
| | | AKT1 | | AKT2 | | AKT3 | |
| Compd | R | Inactive | Active | Inactive | Active | Inactive | Active |
| 23a | 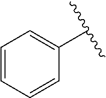 [SMILES: *c1ccccc1] | 0.0027 | 0.0050 | 0.014 | 0.0045 | 0.0081 | 0.036 |
| 23b | 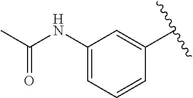 [SMILES: *c1cccc(NC(C)=O)c1] | 0.0028 | 0.0023 | 0.0071 | 0.0032 | 0.0054 | 0.022 |
| 23 | 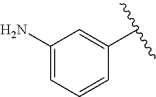 [SMILES: *c1cccc(N)c1] | 0.0019 | 0.0026 | 0.0054 | 0.0028 | 0.0040 | 0.0040 |
| 23c | 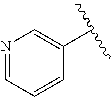 [SMILES: *c1cccnc1] | 0.006 | 0.034 | 0.0025 | 0.030 | 0.026 | 0.34. |

[symbol]

indicates data missing or illegible when filed

In an example embodiment, the kinase inhibitor is ARQ 751. In one example embodiment, the kinase inhibitor is any inhibitor from Ashwell, M. A., et al. “Discovery and Optimization of a Series of 3-(3-Phenyl-3H-Imidazo[4,5-b]Pyridin-2-Yl)Pyridin-2-Amines: Orally Bioavailable, Selective, and Potent ATP-Independent Akt Inhibitors.” J. Med. Chem. 2012, 55 (11), 5291-5310 or any derivative thereof with specific mention of Tables 3, 4, 6, and 8, reproduced below for reference:

TABLE 3
Akt1 Biochemical and Biophysical Results for.Scaffold 24

[symbol]

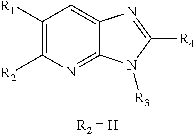

| | | | | A [symbol] | |
|---|---|---|---|---|---|
| | | | | [symbol] T [symbol] | IC₅₀ |
| Compd | R [symbol] | R [symbol] | R [symbol] | (° C.) | (μM) |
| 3a | H | 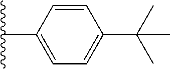 [SMILES: *c1ccc(C(C)(C)C)cc1] | 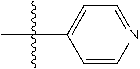 [SMILES: *c1ccncc1] | no shift | >300 |
| 3b | H | 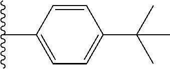 [SMILES: *c1ccc(C(C)(C)C)cc1] | 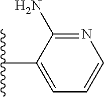 [SMILES: *c1cccnc1N] | 1.9 | 3.9 |
| 3c | H | 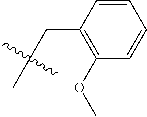 [SMILES: *Cc1ccccc1OC] | 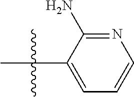 [SMILES: *c1cccnc1N] | no shift | 38 |
| 3d | Br | 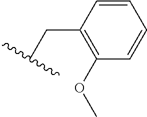 [SMILES: *Cc1ccccc1OC] | 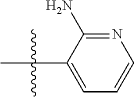 [SMILES: *c1cccnc1N] | no shift | > [symbol] |
| 3e | Br | 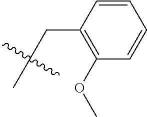 [SMILES: *Cc1ccccc1OC] | 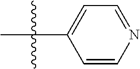 [SMILES: *c1ccncc1] | no shift | > [symbol] |
| 3f | [symbol] | 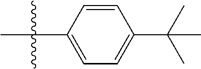 [SMILES: *c1ccc(C(C)(C)C)cc1] | 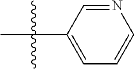 [SMILES: *c1cccnc1] | no shift | [symbol] |
| 3g | Br | 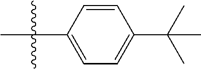 [SMILES: *c1ccc(C(C)(C)C)cc1] | 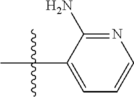 [SMILES: *c1cccnc1N] | 0.9 | > [symbol] |
| 3h | H | 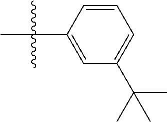 [SMILES: *c1cccc(C(C)(C)C)c1] | 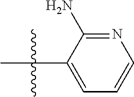 [SMILES: *c1cccnc1N] | no shift | >300 |
| 5a | H | 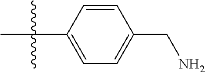 [SMILES: *c1ccc(CN)cc1] | 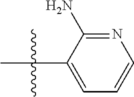 [SMILES: *c1cccnc1N] | 2.6 | 0.93 |
| 5b | H | 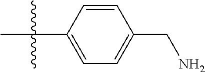 [SMILES: *c1ccc(CN)cc1] | 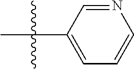 [SMILES: *c1cccnc1] | no shift | 8.8 |
| 5c | H | 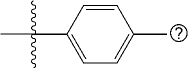 [SMILES: *c1ccc(C)cc1] | 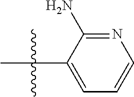 [SMILES: *c1cccnc1N] | 3.7 | [symbol] |
| 5d | H | 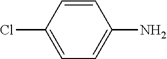 | 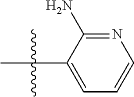 [SMILES: *c1cccnc1N] | 4.1 | 0. [symbol] |

[symbol]

indicates data missing or illegible when filed

TABLE 4
Akti Structure-Activity Relationship For Para-
Substituents

[symbol]

| | | [symbol] | |
|---|---|---|---|
| | | [symbol] T [symbol] | IC₅₀ |
| Compd | 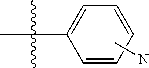 | (° C.) | (μM) |
| 6 [symbol] | 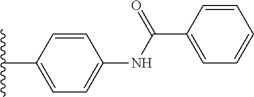 [SMILES: *c1ccc(NC(=O)c2ccccc2)cc1] | 7.3 | 0.014 |
| 7 | 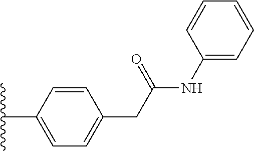 [SMILES: *c1ccc(CC(=O)Nc2ccccc2)cc1] | 7.9 | 0.028 |
| 8a | 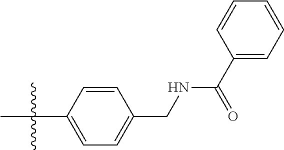 [SMILES: *c1ccc(CNC(=O)c2ccccc2)cc1] | 7.7 | 0.023 |
| 8b | 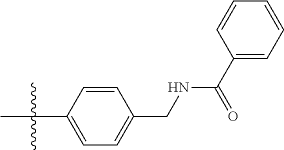 [SMILES: *c1ccc(CNC(=O)c2ccccc2)cc1] | 5. [symbol] | 0.25 |
| 8c | 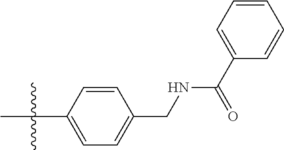 [SMILES: *c1ccc(CNC(=O)c2ccccc2)cc1] | 4.7 | 0.66 |
| 8d | 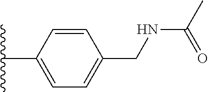 [SMILES: *c1ccc(CNC(C)=O)cc1] | 2.3 | 0.81 |
| 8g | 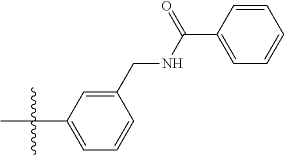 [SMILES: *c1cccc(CNC(=O)c2ccccc2)c1] | no shift | 13 |
| 8h | 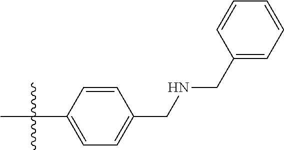 [SMILES: *c1ccc(CNCc2ccccc2)cc1] | 4.9 | 0.26 |

[symbol]

indicates data missing or illegible when filed

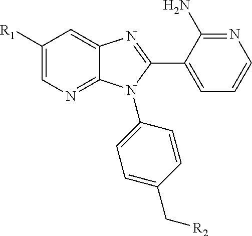

| | | | [symbol] | | | | | |
|---|---|---|---|---|---|---|---|---|
| | | | [symbol] | IC₅₀ | [symbol] | IC₅₀ | [symbol] | IC₅₀ |
| Compd | R [symbol] | R [symbol] | (° C.) | (μM) | (° C.) | (μM) | (° C.) | (μM) |
| 7 | H | 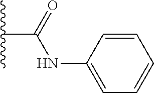 [SMILES: *C(=O)Nc1ccccc1] | 6.7 | 0.028 | 1.2 | 6.70 | no shift | 22 |
| 8 [symbol] | H | 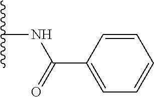 [SMILES: *NC(=O)c1ccccc1] | 6.4 | 0.023 | 1.3 | 0. [symbol] | no shift | 24 |
| 8 [symbol] | Br | 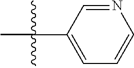 [SMILES: *c1cccnc1] | 8.1 | 0.0 [symbol] | 3.0 | 0.13 | 1.2 | >10 |
| 9 | 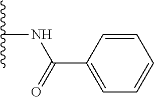 [SMILES: *NC(=O)c1ccccc1] | 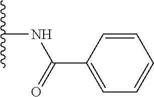 [SMILES: *NC(=O)c1ccccc1] | 6.0 | [symbol] | 3.3 | 0. [symbol] | no shift | >100 |
| 12d | 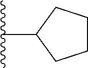 [SMILES: *C1CCCC1] | 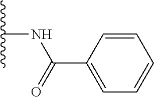 [SMILES: *NC(=O)c1ccccc1] | [symbol] .61 | 0.0 [symbol] | 1.2 | [symbol] | no shift | >100 |
| 12e | 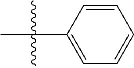 [SMILES: *c1ccccc1] | 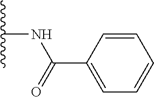 [SMILES: *NC(=O)c1ccccc1] | 9.2 | 0.008 | 4.8 | 0.030 | 1.6 | 0.66 |
| 12g | 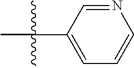 [SMILES: *c1cccnc1] | 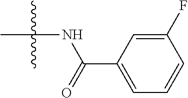 [SMILES: *NC(=O)c1cccc(F)c1] | [symbol] .4 | 0.0 [symbol] | [symbol] .4 | 0.02 [symbol] | 1.5 | 0.40 |

[symbol]

indicates data missing or illegible when filed

or

TABLE 8
In Vivo Pharmacokinetic and Pharmacodynamic
Results For 12e and 12j^{a}

| | | | % inhibition | | | plasma | tumor |
|---|---|---|---|---|---|---|---|
| | po dose | time | p-Akt | p-Akt | p-p70S6 | concn | concn |
| compd | (mg/kg) | (h) | (S473) | (T308) | (T389) | (μM) | (μM) |
| 12e | 100 | 2 | 82 | 25 | 76 | 14.1 | 8.4 |
| 12j | 250 | 2 | 62 | 57 | 90 | 2.0 | 1.2 |
| | | 4 | 51 | 67 | 86 | 1.2 | 0.6. |

In an example embodiment, the kinase inhibitor is borussertib with the formula:

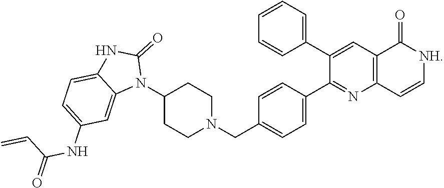 [SMILES: C=CC(=O)Nc1ccc2[nH]c(=O)n(C3CCN(Cc4ccc(-c5nc6cc[nH]c(=O)c6cc5-c5ccccc5)cc4)CC3)c2c1]

Borussertib is a covalent-allosteric inhibitor of AKT, with an IC₅₀ of 0.8 m< and a Kᵢ of 2.2 nM for AKT^{wt}. The EC₅₀ values for Borussertib are 191±90 nM, 48±15 nM, 5±1 nM, 277±90 nM, 373±54 nM, 7770±641 nM in AN3CA (endometrium), T47D (breast), ZR-75-1 (breast), MCF-7 (breast), BT-474 (breast), and KU-19-19 (bladder) cell lines, respectively. In an aspect, the allosteric inhibitor can be according to Table reproduced below:

TABLE 3
Biochemical evaluation of covalent-alosteric [symbol] inhibitors.

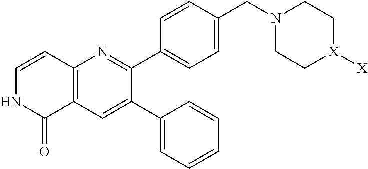

| | | | [symbol] | | | |
|---|---|---|---|---|---|---|
| Cpd | R | X | IC₅₀ (nM) | K [symbol] (nM) | k [symbol] (min⁻ [symbol] ) | k [symbol] (nM⁻ [symbol] s⁻ [symbol] ) |
| 1 | 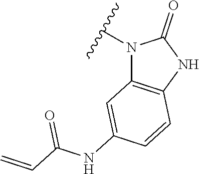 [SMILES: *n1c(=O)[nH]c2ccc(NC(=O)C=C)cc21] | C | 0.8 ± 0.3 | 2.3 ± 6.3 | 0.111 ± 0.020 | 0.853 ± 0.638 |
| 24a | 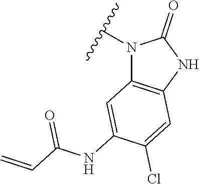 [SMILES: *n1c(=O)[nH]c2cc(Cl)c(NC(=O)C=C)cc21] | C | 1.2 ± 0.3 | 4.1 ± 0.7 | 0.110 ± 0.023 | 0.447 ± 0.074 |
| 24b | 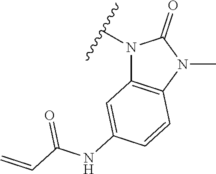 [SMILES: *n1c(=O)n(C)c2ccc(NC(=O)C=C)cc21] | C | 3.0 ± 0.3 | 10.7 ± 0.5 | 0.221 ± 0.016 | 0.190 ± 0.025 |
| 24c | 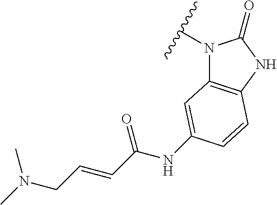 [SMILES: *n1c(=O)[nH]c2ccc(NC(=O)/C=C/CN(C)C)cc21] | C | 18.1 ± 4.9 | 33.0 ± 2.4 | 0.030 ± 0.009 | 0.625 ± 0.005 |
| 27 | 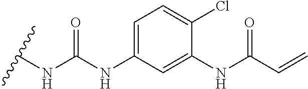 [SMILES: *NC(=O)Nc1ccc(Cl)c(NC(=O)C=C)c1] | C | 9.1 ± 1.5 | 17.5 ± [symbol] .6 | 0.081 ± 0.019 | [symbol] ± [symbol] |

[symbol]

indicates data missing or illegible when filed

Varying of the scaffold can be according to the following scheme:

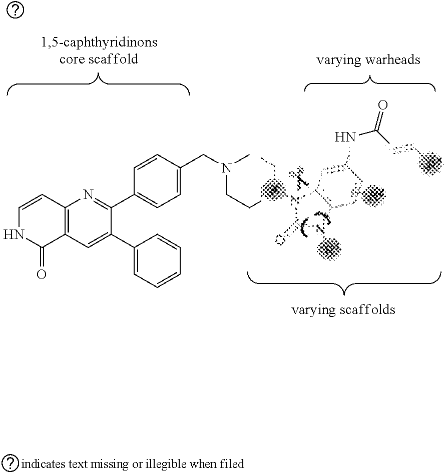

In an example embodiment the AKT inhibitor is Lactoquinomycin according to the formulas:

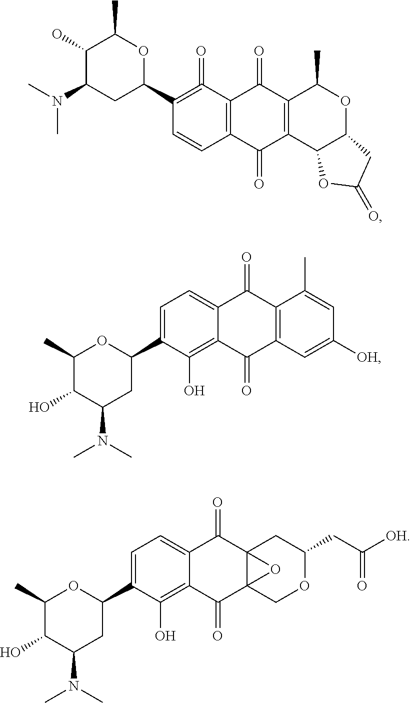

or any derivative thereof, see e.g. “Lactoquinomycin C and D, Two New Medermycin Derivatives from the Marine-Derived Streptomyces Sp. SS17A.” Natural Product Research 2019, 34 (9), 1213-1218. In an example embodiment, the Lactoquinomycin is Medermycin.

In an example embodiment, the AKT inhibitor is BIND-2206, also known as MK-2206 or NSC-749607, according to the formula:

with the following properties:

| | |
|---|---|
| Hydrogen bond acceptors | 4 |
| Hydrogen bond donors | 2 |
| Rotatable bonds | 3 |
| Topological polar surface area | 89.07 |
| Molecular weight | 407.17 |
| XLogP | 5.13 |
| No. Lipinski's rules broken | 1. |

AKT moieties can be synthesized according to the guidance and design provided herein in view of AKT binding moieties as disclosed, for example, in Panicker et al. Adv Exp Med Biol 1163:253-278 (2019); Botello-Smith et al. PLOS Comp Biol 13(8): e1005711 (2017); Mou et al. Chem Biol Drug Des 89(5):723-731 (2017); Ruiz-Carillo et al. Sci Rep 8:7365 (2018), and Budas et al. Biochem Soc Trans 35:1021-1026 (2007). Further information on AKT allosteric inhibitors may be found in Wu, W.-I., et al. “Crystal Structure of Human AKT1 with an Allosteric Inhibitor Reveals a New Mode of Kinase Inhibition.” PLoS ONE 2010, 5 (9), e12913.

### ALK Binding Moiety

In one example embodiment, the protein binding moiety is an ALK kinase binding moiety. In one example embodiment, the ALK binding moiety is an inhibitor or activator. Anaplastic Lymphoma Kinase also known as ALK tyrosine kinase receptor or CD246. ALK participates in cellular communication and the development and function of the nervous system. Upon binding of a ligand, a full-length receptor ALK dimerizes, changes conformation, and autoactivates its own kinase domain. An autoactivated ALK dimer will phosphorylate other ALK receptors on specific tyrosine amino acid residues. ALK phosphorylated residues are binding sites for the recruitment of several adaptor. In one example embodiment, the ALK inhibitor comprises Crisotinib, Ceritinib, Alectinib, Brigatinib, or Lorlatinib.

In an embodiment, the ALK inhibitor is CH5424802, according to the formula:

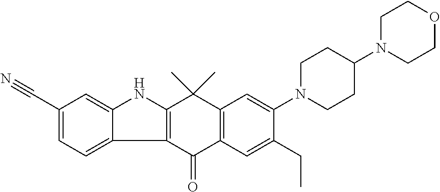 [SMILES: CCc1cc2c(cc1N1CCC(N3CCOCC3)CC1)C(C)(C)c1[nH]c3cc(C#N)ccc3c1C2=O]

or a derivative thereof.

### BTK Binding Moiety

In one example embodiment, the protein binding moiety is an BTK kinase binding moiety. In one example embodiment, the BTK binding moiety is an inhibitor or activator. Bruton's tyrosine kinase (Btk) is involved in multiple signaling cascades, and plays a role in B-cell development and oncogenic signaling. See, e.g. Singh et al., 2018; Pal et al., 2018. In an example embodiment, the BTK inhibitor is ibrutinib, acalabrutinib or a derivative thereof.

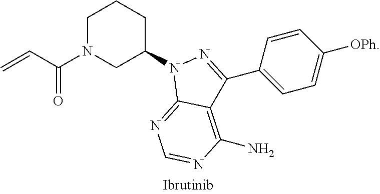 [SMILES: C=CC(=O)N1CCC[C@@H](n2nc(-c3ccc(Oc4ccccc4)cc3)c3c(N)ncnc32)C1]

Exemplary derivatives include

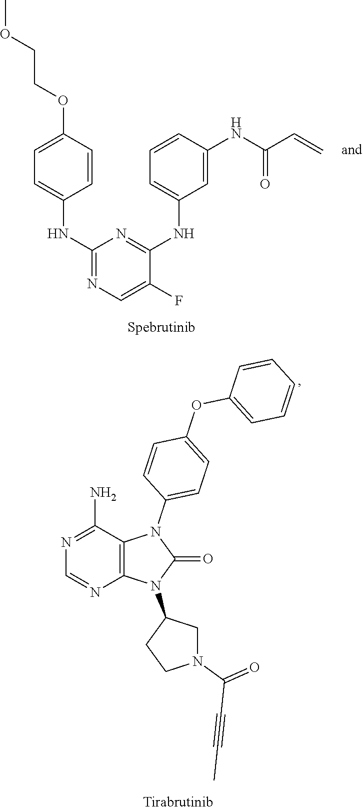 [SMILES: C=CC(=O)Nc1cccc(Nc2nc(Nc3ccc(OCCOC)cc3)ncc2F)c1 ; CC#CC(=O)N1CC[C@@H](n2c(=O)n(-c3ccc(Oc4ccccc4)cc3)c3c(N)ncnc32)C1]

as detailed in Liclican et al, Biochimica et Biophysica Acta (BBA) 1864(4): 129531, DOI: 10.1016/j.bbagen.2020.129531.

In one example embodiment, the BTK activator is selected from

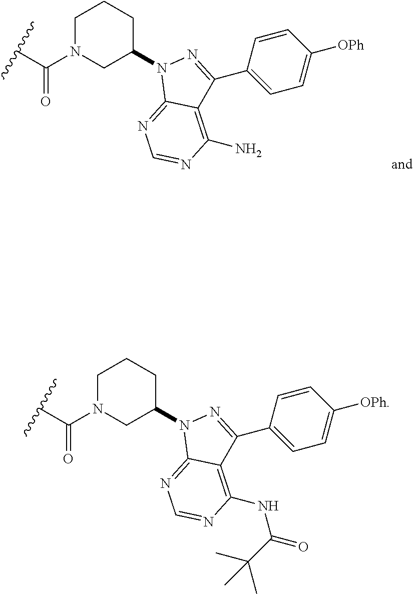 [SMILES: *C(=O)N1CCC[C@@H](n2nc(-c3ccc(Oc4ccccc4)cc3)c3c(N)ncnc32)C1 ; *C(=O)N1CCC[C@@H](n2nc(-c3ccc(Oc4ccccc4)cc3)c3c(NC(=O)C(C)(C)C)ncnc32)C1]

In one example embodiment, the BTK activator moiety is provided with a targeting moiety of

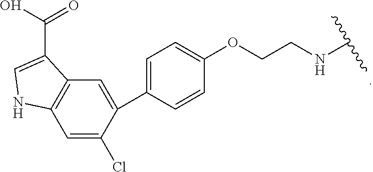 [SMILES: *NCCOc1ccc(-c2cc3c(C(=O)O)c[nH]c3cc2Cl)cc1]

In an example embodiment, the BTK inhibitor is according to the formula:

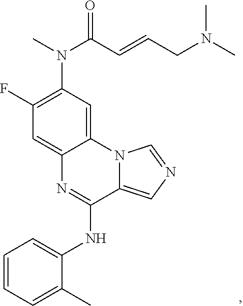 [SMILES: Cc1ccccc1Nc1nc2cc(F)c(N(C)C(=O)/C=C/CN(C)C)cc2n2cncc12]

or a derivative thereof.

### FLT3 Binding Moiety

In one example embodiment, the protein binding moiety is an FLT3 kinase binding moiety. In one example embodiment, the FLT3 binding moiety is an inhibitor or activator. FMS-like tyrosine kinase 3 (FLT3) is a receptor tyrosine kinase that belongs to the subclass III family. FLT3 contain five immunoglobulin-like domains in the extracellular region and an intracellular tyrosine kinase domain split in two by a specific hydrophilic insertion. In one example embodiment, the FLT3 inhibitor comprises Midostaurin, Gilterinib, or a derivative thereof.

### JAK Binding Moiety

Janus kinases (JAKs) are a group of intracellular, non-receptor tyrosine kinases. They transduce cytokine-mediated signals via the JAK-STAT pathway. Adjacent to the cell membrane, JAKs come together with a proline-rich region of the intracellular domain. The JAKs autophosphorylate each other, which causes a conformation change of the JAKs. This enables the JAKs to transduce the intracellular signal by further phosphorylating and activating transcription factors (i.e., STATs). The JAK family included JAK1, JAK2, JAK3, and tyrosine kinase 2 (TYK2).

In one example embodiment, the protein binding moiety is an JAK2 kinase binding moiety. In one example embodiment, the JAK2 binding moiety is an inhibitor or activator. Janus kinase 2 (JAK2) is a non-receptor tyrosine kinase and belongs to the Janus kinase family. JAK2 lacks the Src homology binding domains, SH2 and SH3, but includes seven JAK homology domains, JH1-JH7. In one example embodiment, the JAK2 Inhibitor comprises Ruxolitinib, also known as INCB018424, according to the formula:

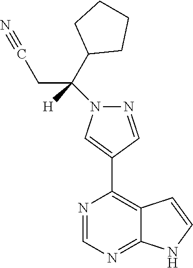 [SMILES: N#CC[C@H](C1CCCC1)n1cc(-c2ncnc3[nH]ccc23)cn1]

In another example embodiment, the JAK2 inhibitor is Tasocitinib, also known as CP690550, according to the formula:

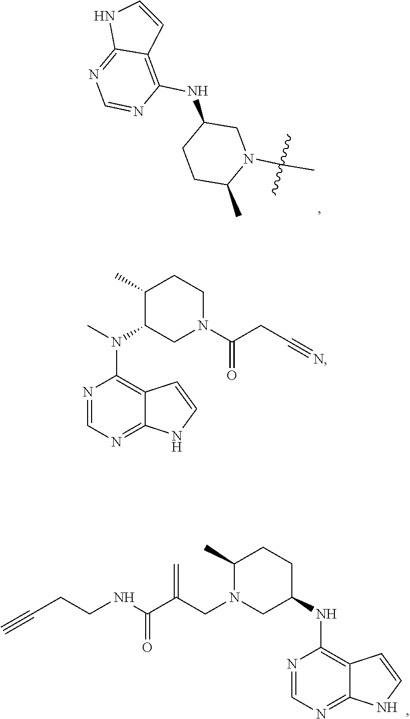 [SMILES: *N1C[C@H](Nc2ncnc3[nH]ccc23)CC[C@@H]1C ; C[C@@H]1CCN(C(=O)CC#N)C[C@@H]1N(C)c1ncnc2[nH]ccc12 ; C#CCCNC(=O)C(=C)CN1C[C@H](Nc2ncnc3[nH]ccc23)CC[C@@H]1C]

or a derivative thereof and the yne-group represents the bonding point for the remaining chimeric small molecule.

JAK3 functions in signals transduction by receptors that comprise the common gamma chain (γc) of the type I cytokine receptor family. JAK3 is generally expressed in hematopoietic and epithelial cells, such as T cells and NK cells for example. In an example embodiment, the kinase binding moiety is an JAK3 binding molecule. In an example embodiment, the JAK3 binding moiety is an inhibitor or activator. In example embodiments, the JAK3 inhibitor is according to the formula:

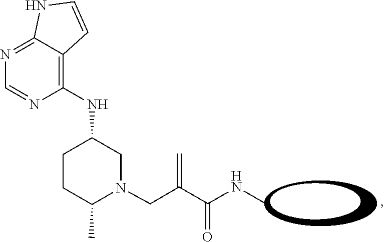 [SMILES: C=C(CN1C[C@@H](Nc2ncnc3[nH]ccc23)CC[C@H]1C)C(=O)NC]

or a derivative thereof and the oval shape represents the remaining chimeric small molecule. In example embodiments, the JAK3 inhibitor is according to the formula:

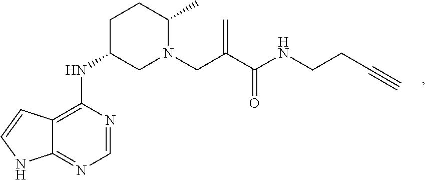 [SMILES: C#CCCNC(=O)C(=C)CN1C[C@H](Nc2ncnc3[nH]ccc23)CC[C@@H]1C]

or a derivative thereof and the yne-group/hexagon represents the bonding point for the remaining chimeric small molecule.

### AURKA Binding Moiety

In one example embodiment, the protein binding moiety is an AURKA kinase binding moiety. In one example embodiment, the AURKA binding moiety is an inhibitor or activator. Aurora A kinase (AURKA) is a member of Setr/Thr kinases whose orthologous control progression through miotic cell division. The other members of the Aurora family are Aurora B and C and they all share a relatively conserved kinase catalytic domain at the carboxy-(C) terminus. In an example embodiment, the Aurora A inhibitor is AurkinA with the formula:

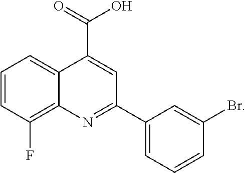 [SMILES: O=C(O)c1cc(-c2cccc(Br)c2)nc2c(F)cccc12]

AurkinA has an IC₅₀, in μM, of 12.7 and Kᵢ, in μM, of 2.7.

In an example embodiment, the Aurora A inhibitor is AA29 with the formula:

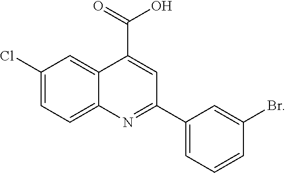 [SMILES: O=C(O)c1cc(-c2cccc(Br)c2)nc2ccc(Cl)cc12]

AA29 has an IC₅₀, in μM, of 34.4 and Kᵢ, in μM, of 7.4.

In an example embodiment, the Aurora A inhibitor is AA30 with the formula:

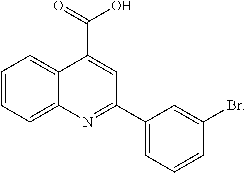 [SMILES: O=C(O)c1cc(-c2cccc(Br)c2)nc2ccccc12]

AA30 has an IC₅₀, in μM, of 25.6 and Kᵢ, in μM, of 5.5. In an aspect, the compound can be according to

| | (Iso)quinoline | Aromatic | Average | Calculated |
|---|---|---|---|---|
| Compound | core | group | IC₅₀/μM | K [symbol] /μM |
| 3 | 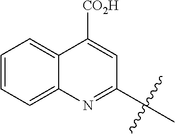 [SMILES: *c1cc(C(=O)O)c2ccccc2n1] | 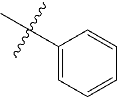 [SMILES: *c1ccccc1] | 289 | 62.5 |
| 24 | | 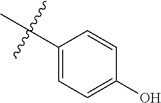 [SMILES: *c1ccc(O)cc1] | >500 | >100 |
| 25 | | 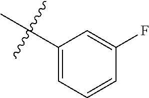 [SMILES: *c1cccc(F)c1] | 75.9 | 16.5 |
| AA30 | | 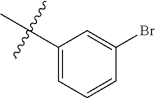 [SMILES: *c1cccc(Br)c1] | 25.6 | 5.5 |
| 33 | | 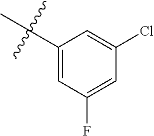 [SMILES: *c1cc(F)cc(Cl)c1] | 20.5 | 4.4 |
| 32 | | 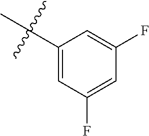 [SMILES: *c1cc(F)cc(F)c1] | 35.0 | 7.8 |
| 34 | | 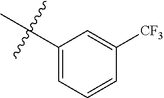 [SMILES: *c1cccc(C(F)(F)F)c1] | 26.5 | 507 |
| 31 | 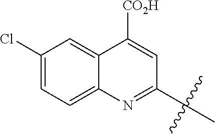 [SMILES: *c1cc(C(=O)O)c2cc(Cl)ccc2n1] | 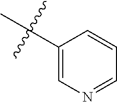 [SMILES: *c1cccnc1] | 163 | 35.5 |
| 25 | | 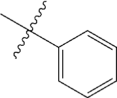 [SMILES: *c1ccccc1] | 205 | 44.7 |
| 27 | | 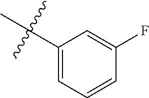 [SMILES: *c1cccc(F)c1] | 107 | 23.3 |
| AA29 | | 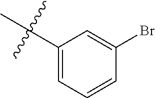 [SMILES: *c1cccc(Br)c1] | 34.4 | 7.4 |
| 28 | 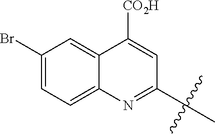 [SMILES: *c1cc(C(=O)O)c2cc(Br)ccc2n1] | 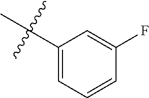 [SMILES: *c1cccc(F)c1] | >500 | >100 |
| A [symbol] | 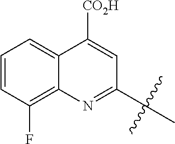 [SMILES: *c1cc(C(=O)O)c2cccc(F)c2n1] | 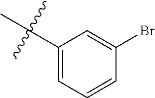 [SMILES: *c1cccc(Br)c1] | 12.7 | 2.7 |
| 50 | 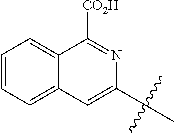 [SMILES: *c1cc2ccccc2c(C(=O)O)n1] | 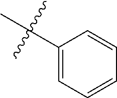 [SMILES: *c1ccccc1] | 2 [symbol] 3 | 46.3 |
| 51 | | 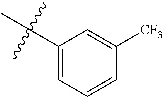 [SMILES: *c1cccc(C(F)(F)F)c1] | 106 | 22.8 |

[symbol]

indicates data missing or illegible when filed

see, Janeček, M., Rossmann, M., Sharma, P. et al. Allosteric modulation of AURKA kinase activity by a small-molecule inhibitor of its protein-protein interaction with TPX2. Sci Rep 6, 28528 (2016). Doi: rep28528, incorporated herein by reference. In one example embodiment, the kinase binding moiety is a monobody that targets Aurora A as described by Zorba A., et al. “Allosteric Modulation of a Human Protein Kinase with Monobodies.” Proc Natl Acad Sci USA 2019, 116 (28), 13937-13942, herein incorporated by reference.

In one example embodiment, the Aurora inhibitor is an Aurora inhibitor or any derivative thereof identified in the US Patent Applicant US20080051327, herein incorporated by reference.

c-MET Binding Moiety

In one example embodiment, the protein binding moiety is an c-MET kinase binding moiety. In one example embodiment, the c-MET binding moiety is an inhibitor or activator. c-MET (mesenchymal-epithelial transition factor) is a receptor tyrosine kinase involved in cellular signaling pathways. After binding with a hepatocyte growth factor, it activates signaling pathways such as proliferation, motility, migration and invasion among others, see e.g. Organ, S. L., et al. “An Overview of the C-MET Signaling Pathway.” Ther Adv Med Oncol 2011, 3, S7-S19. In one example embodiment, the c-MET inhibitor is tivantinib, also referred to as ARQ-197, according to the formula

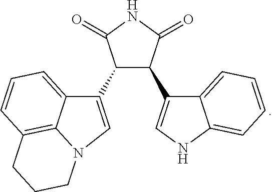 [SMILES: O=C1NC(=O)[C@@H](c2cn3c4c(cccc24)CCC3)[C@@H]1c1c[nH]c2ccccc12]

In an embodiment, the tivantinib binder, or derivative thereof, targets the MET proto-oncogene, receptor tyrosine kinase, is an allosteric inhibitor, and has one or more of the following properties: the tivantinib or derivative thereof, is a non-ATP competitive, MET-specific inhibitor that is 10-100 time more selective for c-Met that other kinases tested (See, Munshi et al., Moll. Can. Ther. doi:10.1158/1535-7163.MCT-09-1173), with an Enzyme IC₅₀ of 50 nM, phosphor-MET IC₅₀ of 100 nM, viability IC₅₀ of 100 nM, and Invasion IC₅₀ of 80 nM, each in NCI-H441 cells. Tivantinib has shown inhibition of growth in breast carcinoma, prostate carcinoma, colon carcinoma and pancreatic carcinoma xenografts as well as inhibit metastasis formation in experimental metastatic models of orthotopic colon cancer xenografts. Additionally, the tivantinib inhibitor has a pKi value of 6.4. These features allow for appropriate selection and modification for design of chimeric small molecules, as detailed elsewhere herein.

In an example embodiment, the c-MET inhibitor is Capmatinib or any derivative thereof.

### DDR Binding Moiety

In one example embodiment, the protein binding moiety is an DDR kinase binding moiety. In one example embodiment, the DDR binding moiety is an inhibitor or activator. Discoidin domain receptor (DDR) belongs to the receptor tyrosine kinase family and are distinguished by the ligand that actives it, fibrillar collagen. Furthermore, their activation and inactivation kinetics are slow and exist as dimers on the cell surface absent their ligand. See e.g. Grither, W. R., et al. “Inhibition of Tumor-Microenvironment Interaction and Tumor Invasion by Small-Molecule Allosteric Inhibitor of DDR2 Extracellular Domain.” Proc Natl Acad Sci USA 2018, 115 (33), E7786-E7794.

In one example embodiment, the DDR inhibitor is selected from:

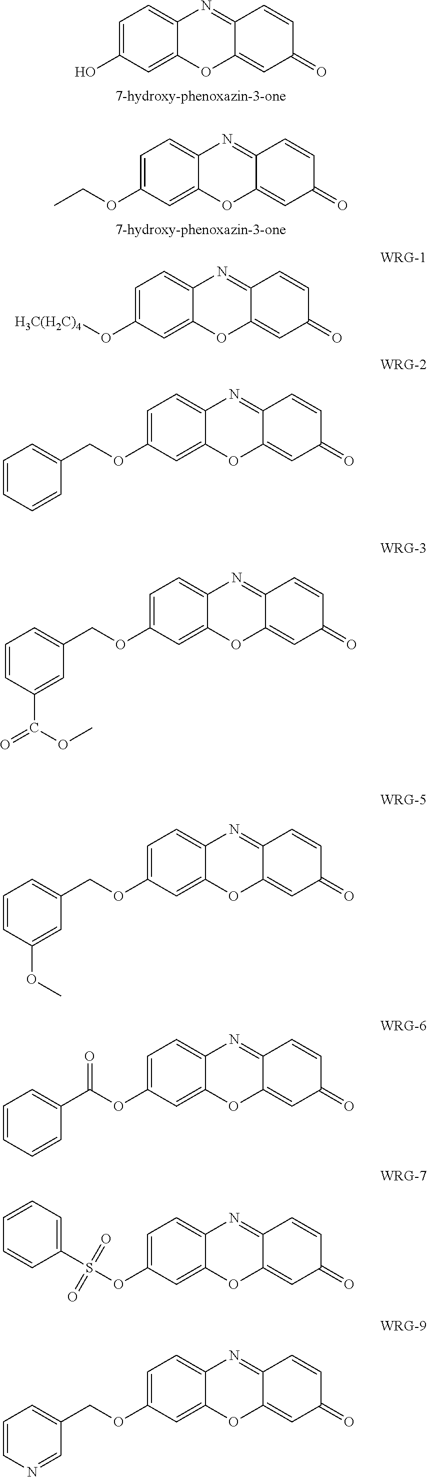

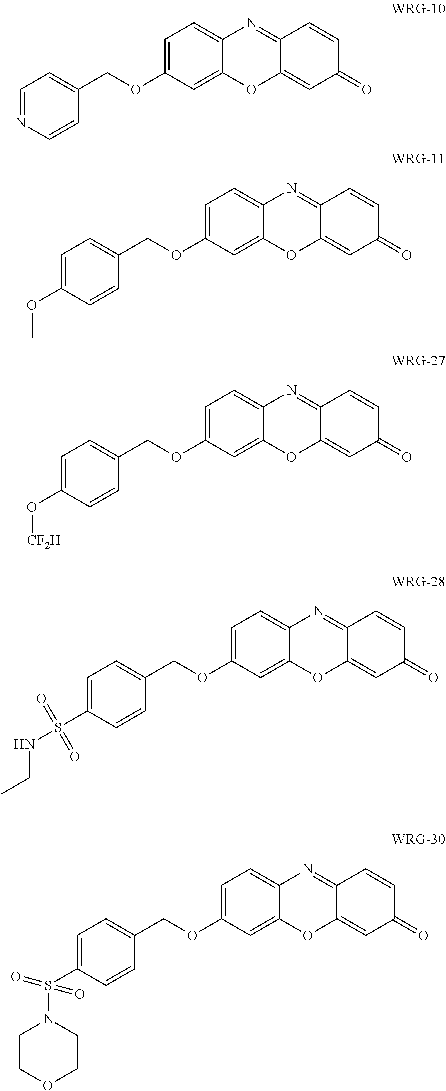 [SMILES: O=c1ccc2nc3ccc(OCc4ccncc4)cc3oc-2c1 ; COc1ccc(COc2ccc3nc4ccc(=O)cc-4oc3c2)cc1 ; O=c1ccc2nc3ccc(OCc4ccc(OC(F)F)cc4)cc3oc-2c1 ; CCNS(=O)(=O)c1ccc(COc2ccc3nc4ccc(=O)cc-4oc3c2)cc1 ; O=c1ccc2nc3ccc(OCc4ccc(S(=O)(=O)N5CCOCC5)cc4)cc3oc-2c1]

or a derivative thereof.

In one example embodiment, the DDR inhibitor is WRG-28, with an IC₅₀ of 230 nM according to the formula:

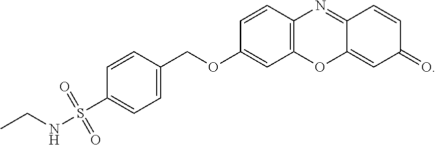 [SMILES: CCNS(=O)(=O)c1ccc(COc2ccc3nc4ccc(=O)cc-4oc3c2)cc1]

In one embodiment, the WRG-28 or derivative thereof, is an extracellularly acting allosteric inhibitor which inhibits receptor-ligand interactions via allosteric modulation of the receptor. WRG-28 has been shown to inhibit tumor invasion and migration as well as tumor-supporting roles of the stroma, and inhibits metastatic breast tumor cell colonization in the lungs by targeting DDR2.

### INSR Binding Moiety

In one example embodiment, the protein binding moiety is an INSR kinase binding moiety. In one example embodiment, the INSR binding moiety is an inhibitor or activator. The insulin receptor (INSR) is located in a plasma membrane glycoprotein and member of the receptor tyrosine kinase (RTK) family that modulates insulin. The INSR family comprises of RTKs including the insulin like growth factor-1 receptor (IGF1R) and insulin receptor-related receptor. See e.g. Hubbard, S. R. “The Insulin Receptor: Both a Prototypical and Atypical Receptor Tyrosine Kinase.” Cold Spring Harbor Perspectives in Biology 2013, 5 (3). In one example embodiment, the kinase binding moiety is an INSR inhibitor. In an example embodiment, the INSR inhibitor is XMetD, also known as RZ-358 or XOMA358, which is a human anti-INSR IgG2 monoclonal antibody. XMetD is a negative allosteric modulator of the INSR. See e.g. Patel P., et al. “A Unique Allosteric Insulin Receptor Monoclonal Antibody That Prevents Hypoglycemia in the SUR-1−/− Mouse Model of KATP Hyperinsulinism.” mAbs 2018, 10 (5), 796-802.

In an embodiment, the protein is an insulin receptor and the binding moiety is RZ-358, also known as XOMA-358 that is fully human negative allosteric modulating insulin receptor antibody. RZ358 can be intravenously administered and binds to a site on the insulin receptor present in the liver, fat and muscle. The RZ358 molecule has high selectivity to the insulin receptor with no IGF-1 interaction and still allows insulin to bind and signal, deepening the insulin signal only when insulin is elevated. Clinical trials have been performed with dosing ranging from 0.1 to 9 mg/kg and has been studied in congenital hyperinsulinism and post-gastric bypass hypoglycemia.

Additional selective allosteric antibodies to the Insulin receptor, including XMetD have been identified using a research platform and can be utilized in small molecules disclosed herein. See, J Journal of Diabetes Science and Technology 2014, 8, 865-873, doi:10.4161/mabs.26871. In an embodiment, the binding moiety is an allosteric insulin receptor antibody, for example XOMA358. Phase 2 clinical trials show ZOMA358 exhibits an inhibition on insulin signaling in patients with improper insulin signaling, including congenital hyperinsulinism. Treatment using the antibody in volunteers ranges from 01.mg/kg to 9 mg/kg. See, Johnson et al., Attenuation of Insulin Action by an Allosteric Insulin Receptor Antibody in Healthy Volunteers. J Clin Endocrinol Metab. 2017 Aug. 1; 102(8):3021-3028. doi: 10.1210/jc.2017-00822.

### IKK Binding Moiety

In one example embodiment, the kinase binding moiety is an IKK kinase binding moiety. In one example embodiment, the IKK binding moiety is an inhibitor or activator. The IKβ kinase (IKK) complex comprises of three subunits: IKKα, IKKβ, and IKKγ/NEMO. The subunits IKKα and IKKβ are catalytic and IKKγ/NEMO is regulatory. See e.g. Karin, M. “The IκB Kinase—a Bridge between Inflammation and Cancer.” Cell Res 2008, 18 (3), 334-342. In an example embodiment, the IKK inhibitor is BMS-345541 according to the formula:

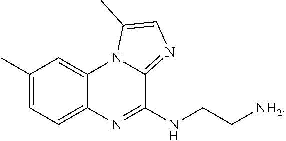 [SMILES: Cc1ccc2nc(NCCN)c3ncc(C)n3c2c1]

mTOR Binding Moiety

In one example embodiment, the protein binding moiety is an mTOR kinase binding moiety. In one example embodiment, the mTOR binding moiety is an inhibitor or activator. Mammalian target of rapamycin (mTOR) is a serine/threonine protein kinase of the PI3K-related protein kinase family. mTOR is large, approximately 300-500 kDa, and contains a conserved kinase catalytic domain. mTOR also includes HEAT repeats, FAT domains, FATC domains, and a FRB (FKBP12/rapamycin-binding) domain that binds the drug rapamycin in complex with its intracellular receptor protein FKBP12. See e.g., Ballou L. M., et. al. “Rapamycin and MTOR Kinase Inhibitors.” J Chem Biol 2008, 1 (1-4), 27-36.

In an example embodiment, the mTOR inhibitor is Sirolimus, also known as Rapamycin, according to the formula:

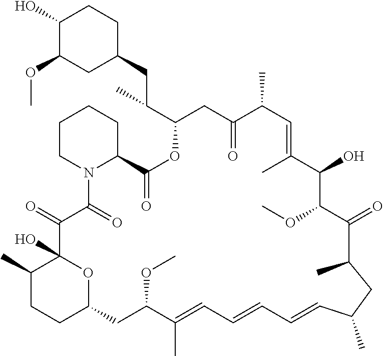 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

and has the following properties

| | |
|---|---|
| Hydrogen bond acceptors | 14 |
| Hydrogen bond donors | 3 |
| Rotatable bonds | [symbol] |
| Topological polar surface area | [symbol] 5.43 |
| Molecular weight | [symbol] 13.5 [symbol] |
| XLogP | 4.32 |
| No. Lipinski's rules broken | 1. |

[symbol]

indicates data missing or illegible when filed

Sirolimus is a macrolide produced by the bacteria Streptomyces hygroscopicus. It has potent immunosuppressive and antiproliferative properties. Sirolimus binds to the FK506 binding protein 12 (FKBP12), creating a complex which inhibits mammalian target of rapamycin (mTOR). Sorlimus inhibition of FKBP prolyl isomerase 1A has a pKᵢ of 9.7.

The FKBP12-sirolimus complex is reported to bind to a site distinct from the kinase domain of mTOR and acts as a negative allosteric modulator of mTOR activity. This action reduces mTOR-induced proliferation of activated T-cells, the cells which would normally be involved in the immunological attack on transplanted tissue. See, Am. J. Health-Syst. Pharm. 2000, 57, 437-448. In vitro studies have been performed and these show that sirolimus inhibits MERS-COV infection of Huh7 cells. This mechanism could also be applied to SAR-COV-2 infection. Sirolimus has been used in renal transplantation.

In an example embodiment the mTOR inhibitor is any inhibitor or derivative thereof encompassed in the International Patent Application WO2014177123, herein incorporated by reference.

### PAK Binding Moiety

In one example embodiment, the protein binding moiety is an p21 kinase binding moiety. In one example embodiment, the PAK binding moiety is an inhibitor or activator. p21-activated kinases (PAKs) are serine/threonine protein kinases. PAKs can be divided into two groups: group I comprising of PAK1-3 and group II comprising of PAK4-6. They are effectors of Rac/Cdc42 GTPases and play an important role in cell proliferation, survival, motility, and angiogenesis. See e.g., Karpov A. S., et al. “Optimization of a Dibenzodiazepine Hit to a Potent and Selective Allosteric PAK1 Inhibitor.” ACS Med. Chem. Lett. 2015, 6 (7), 776-781. In an example embodiment, the PAK inhibitor is compound 3, PMID 26191365, according to the formula:

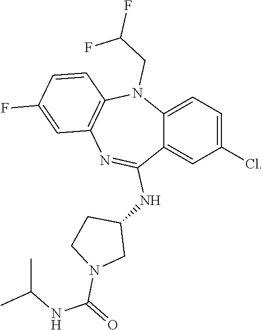 [SMILES: CC(C)NC(=O)N1CC[C@H](NC2=Nc3cc(F)ccc3N(CC(F)F)c3ccc(Cl)cc32)C1]

Compound 3 is a highly selective, negative allosteric regulator of the protein kinase, p21 protein (Cdc42/Rac)-activated kinase 1 with favorable physicochemical properties. Compound 3 binds to a site adjacent to the kinase's ATP binding site. Compound 3 has pK_{d} values of 8.1 and 6.4 for PAK1 (RAC1) and PAK2 (RAC1) respectively. See e.g. Karpov, A. S. et al. “Optimization of a Dibenzodiazepine Hit to a Potent and Selective Allosteric PAK1 Inhibitor.” ACS Med. Chem. Lett. 2015, 6 (7), 776-781, herein incorporated by reference in its entirety. In one example embodiment, the PAK inhibitor is a PAK inhibitor from Karpov ACS Med. Chem. Lett. 2015.
SAR of PAK1 Inhibitors, Selectivity versus other PAK inhibitors is detailed in Table 3 of ACS Med. Chem. Lett, 2015, 6, 776-781 and reproduced below:

TABLE 2
SAR od PAK1 Inhibitors [symbol] Selectivity vs. Other PAK Isoforms

[symbol]

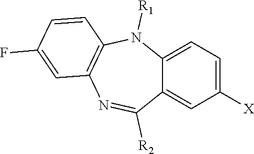

| | | | PAK1dep [symbol] | PAK1 | PAK2 | PAK3 | PAK4 | PAK6 |
|---|---|---|---|---|---|---|---|---|
| Compound | R [symbol] | R [symbol] | IC50 (nM) | Kd (nM) | Kd (nM) | Kd (nM) | Kd (nM) | Kd (nM) |
| 1 | H [symbol] F | 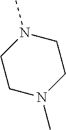 | 12000 | | | | | |
| [symbol] | CH [symbol] Cl | | 900 | | | | | |
| 11 | CH [symbol] CH [symbol] Cl | | 323 | 340 | >40000 | >40000 | >40000 | >40000 |
| 13 | CH [symbol] CH [symbol] Cl | 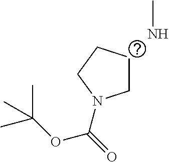 | 190 | 130 | >40000 | >40000 | >40000 | >40000 |
| 2 | CH [symbol] CH [symbol] Cl | 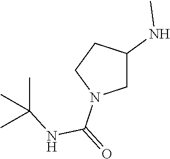 | 18 | 9.9 | 1100 | >40000 | >40000 | |
| 3 | CH [symbol] CH [symbol] Cl | 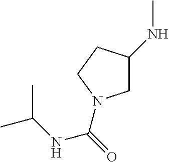 | [symbol] .2 | [symbol] | 400 | | | |

[symbol]

indicates data missing or illegible when filed

In an example embodiment, the PAK inhibitor is IPA-3 according to the formula:

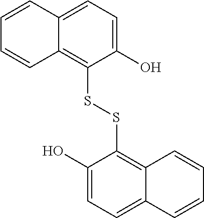 [SMILES: Oc1ccc2ccccc2c1SSc1c(O)ccc2ccccc12]

and has the following properties:

| | |
|---|---|
| Hydrogen bond acceptors | 0 |
| Hydrogen bond donors | 2 |
| Rotatable bonds | 3 |
| Topological polar surface area | 91.06 |
| Molecular weight | 3 [symbol] 0.04 |
| XLogP | 6.1 [symbol] |
| No. Lipinski's rules broken | 1. |

[symbol]

indicates data missing or illegible when filed

IPA-3 is a cell-permeable, non-ATP-competitive, allosteric, and selective inhibitor of p21 protein (Cdc42/Rac)-activated kinase 1 (PAK1). IPA-3 binds covalently to the autoregulatory domain of PAK1, preventing its activation by Cdc42. IPA-3 has a pIC₅₀ of 5.6 for PAK1 (RAC1). See e.g. Viaud, J.; Peterson, J. R. “An Allosteric Kinase Inhibitor Binds the P21-Activated Kinase Autoregulatory Domain Covalently.” Mol Cancer Ther 2009, 8 (9), 2559-2565 and Deacon, S. W., et al. “An Isoform-Selective, Small-Molecule Inhibitor Targets the Autoregulatory Mechanism of P21-Activated Kinase. Chemistry & Biology 2008, 15 (4), 322-331, both herein incorporated by reference in their entirety.

In an example embodiment, the PAK inhibitor is KPT-9274 according to the formula:

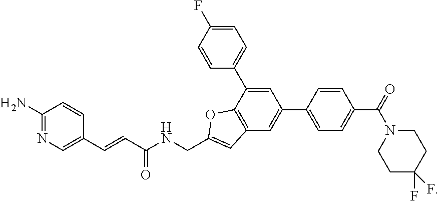 [SMILES: Nc1ccc(/C=C/C(=O)NCc2cc3cc(-c4ccc(C(=O)N5CCC(F)(F)CC5)cc4)cc(-c4ccc(F)cc4)c3o2)cn1]

KPT-9274 is a small molecule that inhibits PAK4 and NAMPT. KPT-9274 acts as an allosteric modulator of PAK4 that does not interfere with the enzyme's kinase activity, in contrast to the PAK kinase inhibitor PF-3758309. KPT-9274 has begun Phase 1 clinical evaluation for non-Hodgkin lymphoma and for solid tumors. KPT-9274 inhibits recombinant human NAMPT with an IC₅₀ of 120 nM in a cell-free assay. KPT-9274 inhibits proliferation of MS751 cervical carcinoma and Z138 B cell acute lymphoblastic leukemia cell lines with IC₅₀ values <100 nM in vitro, and induces shrinkage of Molt-4 (T cell acute lymphoblastic leukemia) xenografts in SCID mice. In addition, KPT-9274 inhibits B-ALL cell lines: KOPN-8; RS4; REH; 697 cells; OP-1; Nalm6; SupB15; SEM with IC₅₀ values, in nM, of 2.4; 5.6; 14.3; 16.7; 18.0; 19.0; 22.6; and >10,000 respectively. KPT-9274 also inhibits PDX B-ALL: LAX2; LAX7R; and ICN13 with IC₅₀ values, in nM, of 19.4; 32.7; and 25.9.

### PDK1 Binding Moiety

In one example embodiment, the protein binding moiety is an PDK1 kinase binding moiety. In one example embodiment, the PDK1 binding moiety is an inhibitor or activator. Phosphoinositide-dependent protein kinase-1 (PDK1) regulates the AGC family of kinases. PDK1 comprises three ligand binding sites: the substrate binding site, the catalytic ATP binding site, and the PDK1 Interacting Fragment (PIF) binding site. The PIF binding site, which is hydrophobic, has two functions: the recruitment of the downstream substrate kinases harboring the hydrophobic motif (HM) and the stimulation of the intrinsic activity of PDK1. See e.g. “The Chemical Diversity and Structure-Based Discovery of Allosteric Modulators for the PIF-Pocket of Protein Kinase PDK1.” Journal of Enzyme Inhibition and Medicinal Chemistry 2019, 34 (1), 361-374. In one example embodiment, the kinase binding moiety is a PDK1 inhibitor. In one example embodiment, the kinase binding moiety is a PDK1 inhibitor. In an example embodiment, the PDK1 inhibitor is PS48 according to the formula:

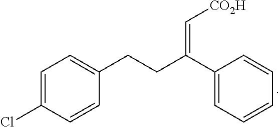 [SMILES: O=C(O)/C=C(/CCc1ccc(Cl)cc1)c1ccccc1]

PS48 has an AC₅₀ value of 8.0 μM. See e.g., Hindie, V., et al. “Structure and Allosteric Effects of Low-Molecular-Weight Activators on the Protein Kinase PDK1.” Nat Chem Biol 2009, 5 (10), 758-764, incorporated by reference in its entirety with specific reference to FIG. 3 depicting binding pocket and Table 1, depicted below:

TABLE 1
Thermodynamic parameters of PDK1 interaction with low-molecular-weight compound activators

| | | | | K [symbol] | K [symbol] | [symbol] H | [symbol] G | [symbol] |
|---|---|---|---|---|---|---|---|---|
| | | Isomer | [symbol] | [symbol] | [symbol] | [symbol] | [symbol] | [symbol] |
| PS48 | 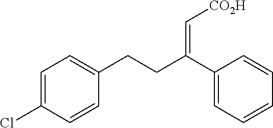 [SMILES: O=C(O)/C=C(/CCc1ccc(Cl)cc1)c1ccccc1] | Z | 1.0 (0.05) | 9.7 × 10 [symbol] (1.2 × 10 [symbol] ) | 10.3 | −1.82 (0.15) | −6.73 (0.08) | 4 [symbol] |
| PS08 | 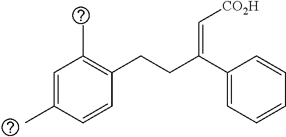 [SMILES: Cc1ccc(CC/C(=C/C(=O)O)c2ccccc2)c(C)c1] | Z | 1.01 (0.05) | 1.62 × 10 [symbol] (0.14 × 10 [symbol] ) | 6.2 | −1.79 (0.08) | −7.15 (0.05) | 5 [symbol] |

[symbol]

indicates data missing or illegible when filed

See also Stroba, A., et al. “3,5-Diphenylpent-2-Enoic Acids as Allosteric Activators of the Protein Kinase PDK1: Structure-Activity Relationships and Thermodynamic Characterization of Binding as Paradigms for PIF-Binding Pocket-Targeting Compounds” J. Med. Chem. 2009, 52 (15), 4683-4693, both incorporated herein by reference in their entirety. Table 1 from Stroba, entitled Effect of Compound son Catalytic Activity of PDK1 and Thermodynamic Characterization of Binding is particularly incorporated by reference and is reproduced below:

| | | Kinase activity assay | | ITC | | | | | |
|---|---|---|---|---|---|---|---|---|---|
| | | A [symbol] | AC [symbol] | K [symbol] | K [symbol] | [symbol] H [symbol] | T [symbol] | [symbol] G | [symbol] H [symbol] |
| No. | Structure | [symbol] | μM | M [symbol] | M [symbol] | kcal/mol | kcal/mol | kcal/mol | % |
| | 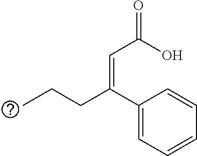 | | | | | | | | |
| | Ar | | | | | | | | |
| 2Z | 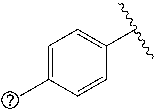 [SMILES: *c1ccc(C)cc1] | 4.0 [symbol] | 8.0 [symbol] | [symbol] | 10.3 | −1.82 | [symbol] | −6.73 | [symbol] |
| 2E | | [symbol] | [symbol] | — | n.b. | | | | |
| 3Z | 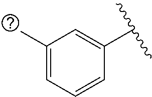 [SMILES: *c1cccc(C)c1] | 2.2 | 9.5 | — | n.d. | | | | |
| 3E | | n.a. | n.a. | — | n.d. | | | | |
| 4Z | 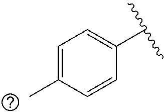 [SMILES: *c1ccc(C)cc1] | 3.3 | 41.0 | — | n.d. | | | | |
| 4E | | [symbol] | [symbol] | — | n.d. | | | | |
| 5Z | 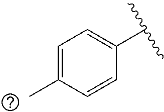 [SMILES: *c1ccc(C)cc1] | 3.9 | 9.8 | 4.78E4 | 20.9 | −3.07 | 3.20 | −6.32 | 48.6 |
| 5E | | [symbol] | [symbol] | — | n.d. | | | | |
| 6Z | 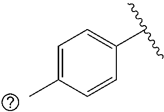 [SMILES: *c1ccc(C)cc1] | 4.4 | 7.1 | 9.88E4 | 10.4 | −1.94 | 4.79 | −6.73 | 28.8 |
| 6E | | [symbol] | [symbol] | — | n.d. | | | | |
| 7Z | 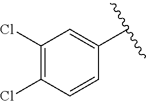 [SMILES: *c1ccc(Cl)c(Cl)c1] | 2.4 | 2.8 | 7.18E4 | 13.9 | −3.71 | 2.80 | −6.56 | 56.6 |
| 7E | | [symbol] | [symbol] | — | n.d. | | | | |
| 8Z | 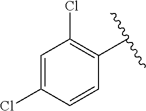 [SMILES: *c1ccc(Cl)cc1Cl] | 2.1 | 4.7 | 1.71E5 | 5.9 | −2.08 | 4.93 | −7.07 | 29.5 |
| 8E | | [symbol] | [symbol] | — | n.d. | | | | |
| 9Z | 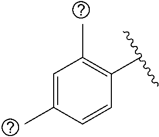 [SMILES: *c1ccc(C)cc1C] | 3.9 | 4.0 | 1.82E5 | 6.2 | −1.79 | 5.19 | −7.1 [symbol] | 25.0 |
| 9E | | [symbol] | [symbol] | — | n.b. | | | | |
| 10Z | 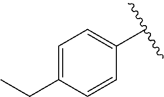 [SMILES: *c1ccc(CC)cc1] | [symbol] | [symbol] | — | n.d. | | | | |
| 10E | | [symbol] | [symbol] | — | n.d. | | | | |
| 11Z | 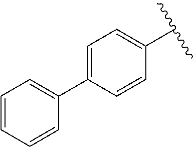 [SMILES: *c1ccc(-c2ccccc2)cc1] | 3.2 | 5.0 | — | n.d. | | | | |
| 11E | | [symbol] | [symbol] | — | n.d. | | | | |
| 12Z | 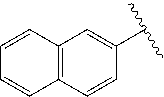 [SMILES: *c1ccc2ccccc2c1] | [symbol] | 8.0 | 7.26E4 | 13.8 | − [symbol] .56 | 1.96 | −6.87 | 58.3 |
| 12E | | [symbol] | [symbol] | — | n.b. | | | | |
| 13Z | 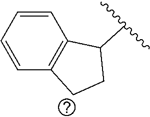 [SMILES: *C1CCc2ccccc21] | 3.1 | 7.6 | 9.63E4 | 10 | −4.09 | 2.60 | −6.73 | 80.8 |
| 13E | | 2.2 | 8.8 | — | n.b. | | | | |

[symbol]

indicates data missing or illegible when filed

In an example embodiment, the PDK1 inhibitor is RS1 according to the formula:

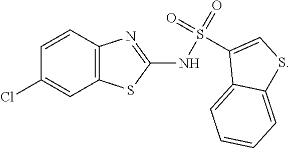 [SMILES: O=S(=O)(Nc1nc2ccc(Cl)cc2s1)c1csc2ccccc12]

RS1 binds to PDK1 selectively. In an example embodiment, the PDK1 inhibitor is RS2 according to the formula:

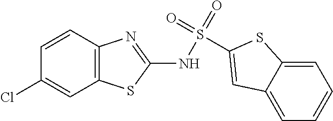 [SMILES: O=S(=O)(Nc1nc2ccc(Cl)cc2s1)c1cc2ccccc2s1]

RS1 and RS2 bound to PDK1 with a Kd of 1.5 μM and 9 μM, respectively.

In an example embodiment, the PDK1 inhibitor is a peptide docking motif (piftide). A piftide is a synthetic peptide. In one example embodiment, the piftide is REPRILSEEEQEMFRDFDYIADW (SEQ ID NO: 3). In an example embodiment, the piftide is a small molecule mimic of the peptide. See e.g. Rettenmaier T. J., et al. “A Small-Molecule Mimic of a Peptide Docking Motif Inhibits the Protein Kinase PDK1. Proc Natl Acad Sci USA 2014, 111 (52), 18590-18595, herein incorporated by reference in its entirety.

In an example embodiment, the PDK1 inhibitor is PS210 according to the formula:

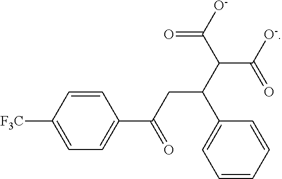

### PTK2/FAK Binding Moiety

In one example embodiment, the protein binding moiety is an PTK2/FAK kinase binding moiety. In one example embodiment, the PTK2/FAK binding moiety is an inhibitor or activator. Protein tyrosine kinase 2 (PTK2), also known as Focal adhesion kinase (FAK), is a non-receptor tyrosine kinase but only distantly related to other tyrosine kinases. PTK2/FAK plays an essential role in mammalian development and numerous physiological functions, most notably cell migration, by integrating signals from integrins as well as growth factor receptors. See e.g. Hirt U. A., et al. “Efficacy of the Highly Selective Focal Adhesion Kinase Inhibitor BI 853520 in Adenocarcinoma Xenograft Models Is Linked to a Mesenchymal Tumor Phenotype.” Oncogenesis 2018, 7 (2).

In an example embodiment, the PTK2/FAK inhibitor is compound 30, PMID 23414845, according to the formula:

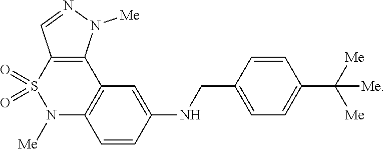 [SMILES: CN1c2ccc(NCc3ccc(C(C)(C)C)cc3)cc2-c2c(cnn2C)S1(=O)=O]

Compound 30 is a selective inhibitor of the tyrosine kinase PTK2 (aka FAK). It is a type III inhibitor in that it binds to an allosteric site, not to the ATP active site of the kinase. In vitro, compound 30 inhibits autophosphorylation of PTK2 with and IC₅₀ of 7.1 μM in prostate cancer cells. PTK2 plays a key role in control of cell proliferation, migration and invasion, and helps regulate resistance to apoptosis. This enzyme is over-expressed in a number of cancers, and reduction of PTK2 activity has growth inhibitory action in vitro and in vivo. These factors make inhibition of PTK2 a novel mechanism for diseases of cellular over-proliferation. In another experiment compound 30 had a pIC₅₀ of 6.2 for the inhibition of PAK2. See e.g. Tomita, N., et al. “Structure-Based Discovery of Cellular-Active Allosteric Inhibitors of FAK.” Bioorganic & Medicinal Chemistry Letters 2013, 23 (6), 1779-1785, herein incorporated by reference in its entirety with specific reference to Tables 1, 3, and 5, depicting evaluation of SAR of substituents with Tables 1 and 5 reproduced below for binders that can be used herein:

TABLE 1
SAR of substituents on the benzene ring

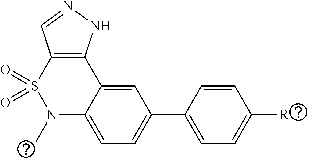

| | | [symbol] AK |
|---|---|---|
| | | IC₅₀ [symbol] |
| Compound | R [symbol] | (μM) |
| 1 | Et | [symbol] .96 |
| 20 | CONH₂ | 3.5 |
| 21 | 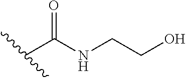 [SMILES: *C(=O)NCCO] | [symbol] .99 |
| 22 | 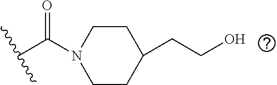 [SMILES: *C(=O)N1CCC(CCO)CC1] | [symbol] .50 |

[symbol]

indicates data missing or illegible when filed

SAR of amino substituents

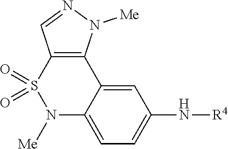

| | | | Cell |
|---|---|---|---|
| | | [symbol] AK | p [symbol] AK |
| | | IC₅₀ [symbol] | IC₅₀ |
| Compound | R [symbol] | (μM) | (μM) |
| 24 | 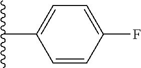 [SMILES: *c1ccc(F)cc1] | 4.3 | >30 |
| 28 | 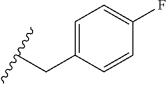 [SMILES: *Cc1ccc(F)cc1] | 0.32 | 19 |
| 29 | 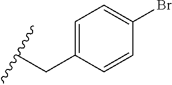 [SMILES: *Cc1ccc(Br)cc1] | 1.4 | >30 |
| 30 | 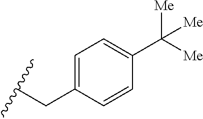 [SMILES: *Cc1ccc(C(C)(C)C)cc1] | 0.54 | 7.1 |

[symbol]

indicates data missing or illegible when filed

### RIPK Binding Moiety

In one example embodiment, the protein binding moiety is an RIPK kinase binding moiety. In one example embodiment, the RIPK binding moiety is an inhibitor or activator. Receptor-interacting protein kinase (RIPK)-1 is involved in RIPK3-dependent and -independent signaling pathways leading to cell death and/or inflammation. See e.g. Degterev A., et al. “Targeting RIPK1 for the Treatment of Human Diseases.” Proc Natl Acad Sci USA 2019, 116 (20), 9714-9722. In an example embodiment, the RIPK inhibitor is RIPA-56 according to the formula.

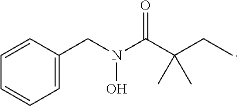 [SMILES: CCC(C)(C)C(=O)N(O)Cc1ccccc1]

RIPA-56 is a highly potent, selective, and metabolically stable type III (allosteric) inhibitor of RIPK1. RIPA-56 is also known as compound 92 in patent WO2016101885, herein incorporated by reference. RIPA-56 is a drug candidate for the treatment of systemic inflammatory response syndrome (SIRS). RIPA-56 is active against human and mouse RIPK1 and is efficacious in animal models. It is devoid of off-target IDO inhibiting activity. RIPA-56 has an pIC₅₀ value of 7.9 for RIPK-1. See e.g. Ren, Y., et al. “Discovery of a Highly Potent, Selective, and Metabolically Stable Inhibitor of Receptor-Interacting Protein 1 (RIP1) for the Treatment of Systemic Inflammatory Response Syndrome.” J. Med. Chem. 2017, 60 (3), 972-986, herein incorporated by reference in its entirety with specific mention of Table 5.

In an example embodiment, the RIP1 inhibitor is according to the formula:

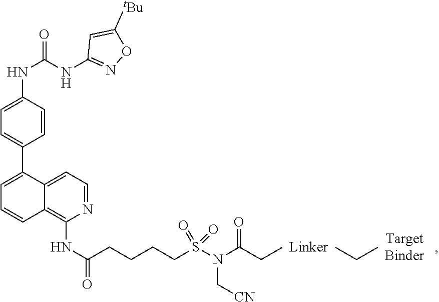

or a derivative thereof and the “Linker” and “Target Binder” represents the remaining chimeric small molecule.

### TYK2 Binding Moiety

In one example embodiment, the protein binding moiety is an TYK2 kinase binding moiety. In one example embodiment, the TYK2 binding moiety is an inhibitor or activator. Tyrosine kinase 2 (TYK2) is a member of the JAK kinase family that regulates signal transduction downstream of receptors. TYK2 pairs with JAK2 to regulate the IL-23/IL-12 pathways and JAK1 to regulate the type I interferon family. In an example embodiment, the TYK2 inhibitor is compound 29 according to the formula:

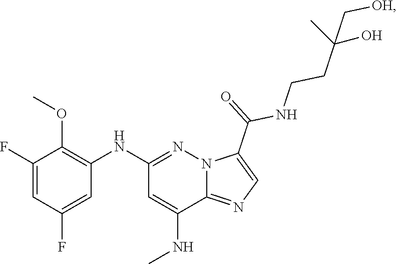 [SMILES: CNc1cc(Nc2cc(F)cc(F)c2OC)nn2c(C(=O)NCCC(C)(O)CO)cnc12]

see e.g. Moslin R., et al. “Identification of Imidazo[1,2-b]Pyridazine TYK2 Pseudokinase Ligands as Potent and Selective Allosteric Inhibitors of TYK2 Signalling.” Med. Chem. Commun. 2017, 8 (4), 700-712.

In an example embodiment, the TYK2 inhibitor is Deucravacitinib, also known as BMS-986165, according to the formula:

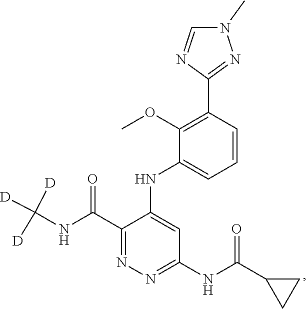

wherein D is deuterium. The deuteromethyl amide group confers selectivity by virtue of binding to a pocket in the TYK2 JH2 ligand binding domain. Deucravacitinib is a selective, orally active, and allosteric inhibitor of the TYK2 where it binds to the JH2 (pseudokinase) domain. Deucravacitinib is kinome selective and does not bind to JAKs1-3 or to the TYK2 JH1 (ATP) binding domain. Deucravacitinib has been shown in inbit IFNα production with an IC₅₀ of 5 nM in vitro. In particular, Deucravacitinib has a pKᵢ value of 10.7 for TYK2 and a pIC₅₀ of 9.7 and 9.0 for TYK2 and JAK1 respectively. Currently Deucravacitinib has advanced to evaluation in clinical studies in patients with systemic lupus erythematosus and ulcerative colitis (both Phase 2) and moderate-to-severe psoriasis (Phase 3). See e.g. Wrobleski, S. T., et al. “Highly Selective Inhibition of Tyrosine Kinase 2 (TYK2) for the Treatment of Autoimmune Diseases: Discovery of the Allosteric Inhibitor BMS-986165.” J. Med. Chem. 2019, 62 (20), 8973-8995 and “Tyrosine Kinase 2 (TYK2) Allosteric Inhibitors To Treat Autoimmune Diseases.” J. Med. Chem. 2019, 62 (20), 8951-8952, incorporated herein by reference in its entirety. Tables 1 and 3 from Wrobleski et al. are specifically incorporated herein by reference. Table 1 shows JAK Family Biochemical Potencies for clinical inhibitors. And Table 3, reproduced below, provides Expansion of C3′ Amide SAR:

TABLE 3
Expansion of C3′ Amida SAR

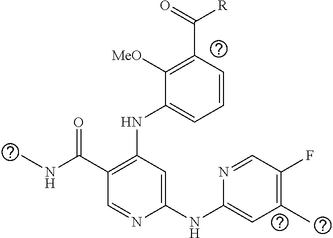

| | | [symbol] | [symbol] | [symbol] |
|---|---|---|---|---|
| | R | [symbol] | [symbol] | [symbol] |
| 22 | —NH₂ | 0.5 ± 0.1 | [symbol] ± [symbol] | [symbol] ± [symbol] |
| 23 | —NHMe | 0.5 ± 0.1 | [symbol] | [symbol] ± [symbol] |
| 24 | —NHEt | 0.4 ± 0.1 | 28 ± 14 | [symbol] ± [symbol] |
| 25 | —N(Me)₂ | [symbol] ± [symbol] | [symbol] ± [symbol] | [symbol] ± [symbol] |
| 26 | —NHCH₂ [symbol] Pr | [symbol] ± [symbol] | [symbol] ± [symbol] | [symbol] ± [symbol] |
| 27 | —NH(CH₂)₂OH | [symbol] ± [symbol] | [symbol] ± [symbol] | 270 ± [symbol] |
| 28 | 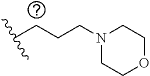 [SMILES: *CCCN1CCOCC1] | [symbol] ± [symbol] | [symbol] ± 45 | [symbol] ± [symbol] |
| 29 | 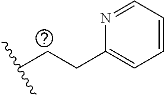 [SMILES: *CCc1ccccn1] | [symbol] ± [symbol] | 22 ± 9 | [symbol] ± 35 |

[symbol]

indicates data missing or illegible when filed

### SHP Binding Moiety

In one example embodiment, the protein binding moiety is an Scr kinase binding moiety. In one example embodiment, the Scr kinase binding moiety is an inhibitor or activator. Src homology 2 (SH2) domain-containing phosphatase 2 (SHP2) belongs to protein tyrosine phosphatase (PTP) family and is a positive transducer of proliferative and antiapoptotic signals from receptor tyrosine kinases. SHP2 is composed of three folded domains and a C-terminal tail. SHP2 modulates phosphatase activity by binding phosphopeptides at the N-terminal SH2 and C-terminal SH2 domains. The PTP domain harbors the catalytic functionality in the conserved signature motif HCX5R. The disordered C-terminal tail contains has a putative regulatory function. See e.g. Marasco M., et al. “Molecular Mechanism of SHP2 Activation by PD-1 Stimulation.” Sci. Adv. 2020, 6 (5), eaay4458. In an example embodiment the SHP3 inhibitor is any from the International Patent Application WO2020076723, herein incorporated by reference.

aPKC Binding Moiety

In one example embodiment, the protein binding moiety is an atypical PKC kinase binding moiety. In one example embodiment, the aPKC binding moiety is an inhibitor or activator. Atypical protein kinase C (aPKC) belongs to the protein kinase C family that are categorized into three groups based on their structure and cofactor regulation. The aPKC isozymes: ζ and λ, are the least understood and differ significantly in structure from the other two classes. First, the C1 domain contains one Cys-rich motif, instead of two. Second, aPKC isozymes do not appear to contain key residues that maintain the C2 fold. In additional feature of aPKCs is they have been reported to not respond to phorbol esters in vivo or in vitro. See e.g. Newton, A. C. “Protein Kinase C: Structure, Function, and Regulation.” Journal of Biological Chemistry 1995, 270 (48), 28495-28498.

In an example embodiment, the aPKC inhibitor is an inhibitor or any derivative thereof identified in the International Patent Application WO2015075051, herein incorporated by reference.

In an example embodiment, the PKC Inhibitor is a PKC-zeta (PKCζ) inhibitor. See, Abdel-Halim, Discovery and Optimization of 1,3,5-Trisubstituted Pyrazolines as Potent and Highly Selective Allosteric Inhibitors of Protein Kinase C-ζ, Journal of Medicinal Chemistry 2014 57 (15), 6513-6530, DOI: 10.1021/jm500521n, incorporated herein by reference in its entirety with specific mention of Tables 1 and 2, reproduced below:

TABLE 1
Inhibition of Recombinant PKC [symbol] and the NF- [symbol] B Pathway in Cells

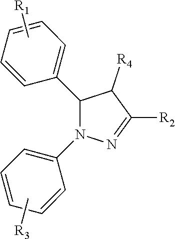

11

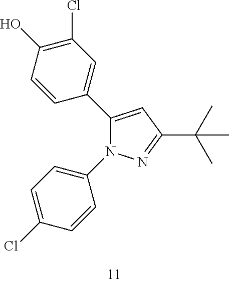 [SMILES: CC(C)(C)c1cc(-c2ccc(O)c(Cl)c2)n(-c2ccc(Cl)cc2)n1]

| | | | | | | | NF [symbol] | |
|---|---|---|---|---|---|---|---|---|
| | | | | | cell [symbol] | | (U [symbol] cells) | |
| | | | | | % [symbol] | IC₅₀ ± SD | % [symbol] | IC₅₀ ± SD |
| compd | R₁ | R₂ | R₃ | R₄ | 62.5 μM [symbol] | (μM) | 5 μM [symbol] | (μM) |
| 1a | 4-OH | tBu | 4-Cl | H | 91.3 | [symbol] ± [symbol] | 75.1 | 3.2 ± 0.22 |
| 1b | 4-OH | tBu | 4-F | H | [symbol] | [symbol] ± [symbol] | [symbol] ± [symbol] | ND [symbol] |
| 1c | 4-OH | tBu | 4-Br | H | 93.2 | [symbol] ± [symbol] | [symbol] | ND |
| 1d | 4-OH | tBu | 4-CF₃ | H | 94.2 | 8.8 ± 0.53 | ND [symbol] | ND |
| 1e | 4-OH | tBu | 4-CH₃ | H | [symbol] | [symbol] ± [symbol] | 62.9 | ND |
| 1f | 4-OH | tBu | 4 [symbol] | H | [symbol] | ND | ND [symbol] | ND |
| 1g | 4-OH | tBu | 4-COOH | H | 47 [symbol] | ND | 40.7 | ND |
| 1h | 4-OH | tBu | 3-Cl | H | 96.8 | 3.2 ± [symbol] | 64.6 | ND |
| 1i | 4-OH | tBu | 3-F | H | 98. [symbol] | 2.2 ± [symbol] | [symbol] | ND |
| 1j | 4-OH | tBu | 3-CF₃ | H | 96.1 | 2.7 ± [symbol] | [symbol] | 2.7 ± [symbol] |
| 1k | 4-OH | tBu | 3-CH₃ | H | 91.7 | 3.5 ± [symbol] | 71.7 | ND |
| 1l | 4-OH | tBu | 2-Cl | H | [symbol] | ND | [symbol] | ND |
| 1m | 4-OH | tBu | 2-F | H | [symbol] | ND | [symbol] | ND |
| 1n | 4-OH | tBu | 2,4- | H | 71.0 | ND | [symbol] | ND |
| | | | dichloro | | | | | |
| 1o | 4-OH | tBu | 2,4- | H | [symbol] | ND | [symbol] | ND |
| | | | difluoro | | | | | |
| 1p | 4-OH | tBu | 2,4- | H | 59.9 | ND | 41.5 | ND |
| | | | dimethyl | | | | | |

[symbol]

indicates data missing or illegible when filed

TABLE 2
Inhibition of PKC [symbol] at 20 μM^{a}

| | PKC [symbol] | | PKC [symbol] | | PKC [symbol] |
|---|---|---|---|---|---|
| | [symbol] | | [symbol] | | [symbol] |
| compd | 20 μM | compd | 20 μM | compd | 20 μM |
| [symbol] a | 0 | 2a | [symbol] .6 | 4a | [symbol] 7.4 |
| [symbol] b | [symbol] | 2b | [symbol] | [symbol] | 29. [symbol] |
| [symbol] | [symbol] | 2c | [symbol] 4 | [symbol] | 0 |
| [symbol] | [symbol] 7.0 | [symbol] | 21.4 | [symbol] | [symbol] |
| [symbol] | [symbol] .9 | [symbol] | 45. [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |

[symbol]

Values are the mean of at least two experiments [symbol] standard deviation < [symbol] %.. [symbol]
indicates data missing or illegible when filed

In an aspect, the binding moiety is a 1,3,5-trisubstituted pyrazoline according to the formula:

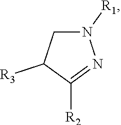

more preferably wherein the binding molecule is selected from

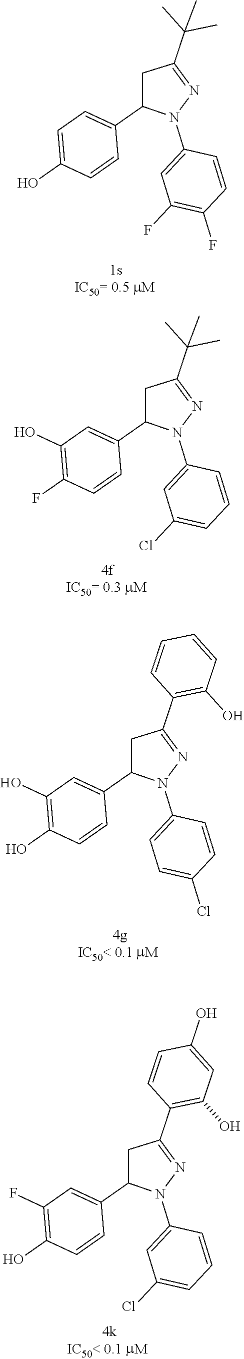

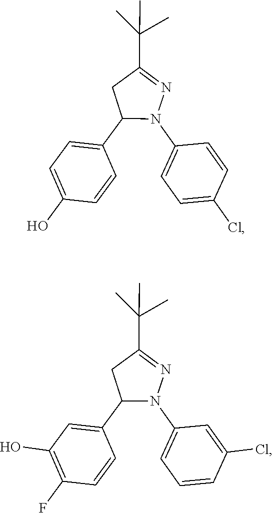 [SMILES: CC(C)(C)C1=NN(c2ccc(Cl)cc2)C(c2ccc(O)cc2)C1 ; CC(C)(C)C1=NN(c2cccc(Cl)c2)C(c2ccc(F)c(O)c2)C1]

or any derivative thereof. 1,3,5-trisubstituted pyrazolines is potent and selective allosteric PKCζ inhibitors. Phenolic group on the 5-phenyl was essential for the inhibitory activity, with a catechol providing the best activity. Presence of a lipophilic (halogen or alkyl) substituent on the 1-phenyl proved to be essential for the generation of high potency.

### SphK Binding Moiety

In one example embodiment, the protein binding moiety is an SphK kinase binding moiety. In one example embodiment, the SphK binding moiety is an inhibitor or activator. Sphingosine kinases (SphKs) are biological lipid kinases that regulate the sphingolipid metabolic pathway and control multiple important cell processes. SphKs are the only enzymes that catalyze ATP-dependent phosphorylation of sphingosine to sphingosine-1-phosphate. SphKs have five conserved domains, C1-C5. The C4 domain appears to be unique SphKs while the C1-C3 domains are also found in ceramide kinase (CERK) and diacylglycerol kinases (DAGK). The two SphK isoforms are SphK1 and SphK2. SphK2 has ˜240 more amino acids than SphK1. See e.g. Cao M., “Sphingosine Kinase Inhibitors: A Patent Review.” Int J Mol Med 2018.

In an example embodiment, the SphK inhibitor is an inhibitor or derivative thereof identified in the International Patent Application WO2014118556, herein incorporated by reference.

### GSK-3 Binding Moiety

In one example embodiment, the protein binding moiety is an GSK-3 kinase binding moiety. In one example embodiment, the GSK-3 binding moiety is an inhibitor or activator. Glycogen synthase kinase-3 (GSK3) comprises two isoforms, GSK3a and GSK3B, that regulate many interactions such as intracellular receptor-coupled signaling proteins, insulin receptors, and several ionotropic neurotransmitter receptors. GSK3 can be found in the cytosol, mitochondria and nucleus, as well as other subcellular compartments. The two key functional domains of GSK3 are the primed-substrate binding domain that recruits substrates to GSK3, and the kinase domain that phosphorylates the substrate. See e.g. Glycogen Synthase Kinase-3 (GSK3): Regulation, Actions, and Diseases. Pharmacology & Therapeutics 2015, 148, 114-131. In an example embodiment, the GSK3 inhibitor is an inhibitor or derivative thereof identified in the U.S. Pat. No. 9,757,369, herein incorporated by reference.

### JNK Binding Moiety

c-Jun N-terminal kinases (JNKs) participate in stress signaling pathways implicated in gene expression, neuronal plasticity, regeneration, cell death, and regulation of cellular senescence. JNKs are one of the three families of MAP kinases. JNKs have three isoforms: JNK1 and JNK2, which is found throughout tissue; and JNK3, which is found in neurons, the heart, and the testis. See e.g. Yarza, R., et al. “C-Jun N-Terminal Kinase (JNK) Signaling as a Therapeutic Target for Alzheimer's Disease.” Front. Pharmacol. 2016, 6. In one example embodiment, the kinase binding moiety is a JNK binding moiety. In one example embodiment, the JNK binding moiety is an inhibitor or activator. In one example embodiment, the JNK inhibitor is compound 10, according to the formula:

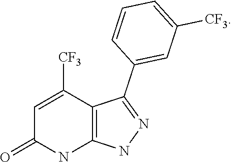 [SMILES: O=c1cc(C(F)(F)F)c2c(-c3cccc(C(F)(F)F)c3)n[nH]c2[nH]1]

Compound 10 has IC₅₀ values, in μM, of: 1.2 in 0.1 mM p38α assay; 0.8 in 0.1 mM MKK6/p38α cascade assay; 1.4 in 0.01 mM p38α/MK2 cascade assay; >100 in 0.1 mM MKK6 assay; >40 in 0.01 mM MK2 assay; >40 in 0.1 mM p38β assay; >40 in 0.1 mM p38γ assay; and >40 in 0.1 mM p28δ assay, see e.g. Comess, K. M., et al. “Discovery and Characterization of Non-ATP Site Inhibitors of the Mitogen Activated Protein (MAP) Kinases.” ACS Chem. Biol. 2011, 6 (3), 234-244, incorporated herein by reference. In an aspect, compound 10 binds the lipid binding pocket. Additional JNK1 non-ATP site inhibitors can also be used in the small molecules disclosed herein, including biary-tetrazole based Jnk-1 Activation inhibitors. In an embodiment, the biaryl-tetrazole based binding moiety for Jnk-1 are selected from Table 2 of ACS Chem. Biol. 2011, 6, 234-244, reproduced below:

| | | IC₅₀ | IC₅₀ | EC₅₀ |
|---|---|---|---|---|
| No. | Structure | (MKK7-Jak [symbol] ) [symbol] | (Jak1) [symbol] | (P- [symbol] ) [symbol] |
| 2 | 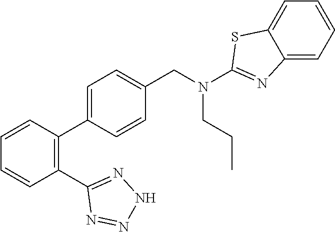 [SMILES: CCCN(Cc1ccc(-c2ccccc2-c2nn[nH]n2)cc1)c1nc2ccccc2s1] | 7.8 | >100 | >30 |
| 3 | 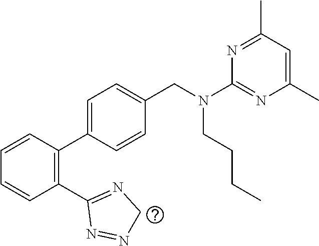 [SMILES: CCCCN(Cc1ccc(-c2ccccc2C2=NCN=N2)cc1)c1nc(C)cc(C)n1] | 7.7 | >10 | >30 |
| 4 | 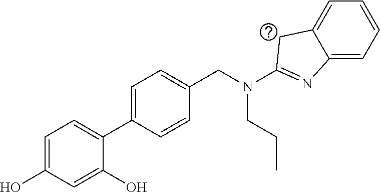 [SMILES: CCCN(Cc1ccc(-c2ccc(O)cc2O)cc1)C1=Nc2ccccc2C1] | 2.9 | >100 | >10 |
| 5 | 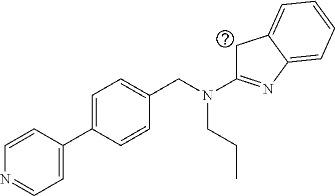 [SMILES: CCCN(Cc1ccc(-c2ccncc2)cc1)C1=Nc2ccccc2C1] | 2.8 | 68.6 | >10 |
| 6 | 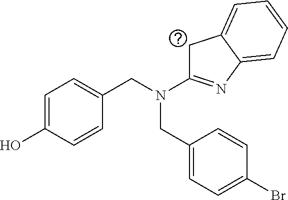 [SMILES: Oc1ccc(CN(Cc2ccc(Br)cc2)C2=Nc3ccccc3C2)cc1] | 3.1 | ND | >10 |
| 7 | 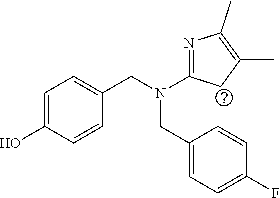 [SMILES: CC1=C(C)N=C(N(Cc2ccc(O)cc2)Cc2ccc(F)cc2)C1] | 3.8 | >100 | 4.0 |
| 8 | 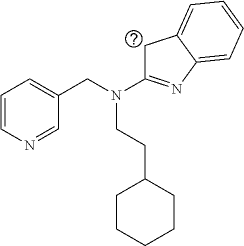 [SMILES: c1cncc(CN(CCC2CCCCC2)C2=Nc3ccccc3C2)c1] | 4.7 | 82.3 | >10 |

[symbol]

indicates data missing or illegible when filed

Further details regarding biaryl-tetrazole affinity and coupled assay data for JNK isoforms and related MAP kinase protein from Table 1 of ACS Chem. Biol. 2011, 6, 234-244 is also provided, and is adapted below for reference

| | | IC₅₀ [symbol] | IC₅₀ [symbol] | [symbol] | [symbol] | [symbol] | [symbol] |
|---|---|---|---|---|---|---|---|
| No. | Stucture | [symbol] | [symbol] | [symbol] | [symbol] | [symbol] | [symbol] |
| 2 | 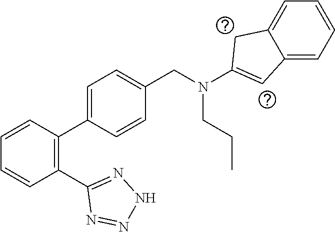 [SMILES: CCCN(Cc1ccc(-c2ccccc2-c2nn[nH]n2)cc1)C1=Cc2ccccc2C1] | >100 | 7.8 | 11 | >50 | 18 | 18 |
| 3 | 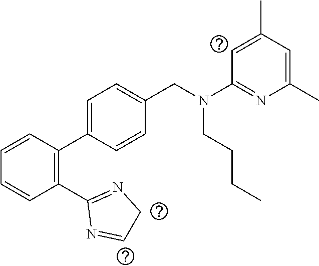 [SMILES: CCCCN(Cc1ccc(-c2ccccc2C2=NCC=N2)cc1)c1cc(C)cc(C)n1] | [symbol] | 7.7 | 16 | >50 | 17 | 32 |

[symbol]

indicates data missing or illegible when filed

In an example embodiment, the binding moiety is selected from:

| Compound | |
|---|---|
| IC₅₀ (μM) JNK1 | |
| IC₅₀ (μM) JNK2 | Structure |
| 1 0.88 ± 0.19 1.2 ± 0.3 | 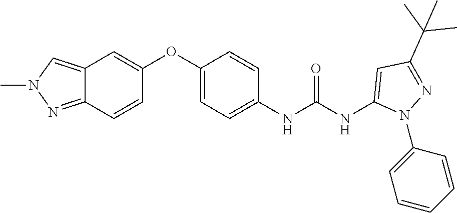 [SMILES: Cn1cc2cc(Oc3ccc(NC(=O)Nc4cc(C(C)(C)C)nn4-c4ccccc4)cc3)ccc2n1] |
| 2 0.48 ± 0.27 2.8 ± 1.1 | 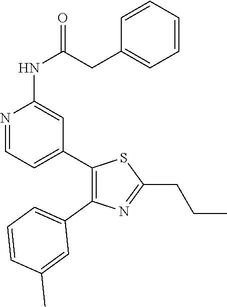 [SMILES: CCCc1nc(-c2cccc(C)c2)c(-c2ccnc(NC(=O)Cc3ccccc3)c2)s1] |
| 3 17 ± 11 15 ± 4 | 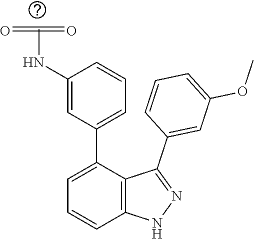 |
| 4 0.070 ± 0.023 0.59 ± 0.15 | 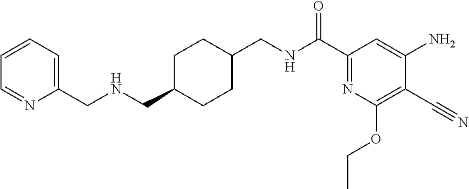 |
| 5 19 ± 5 0.99 ± 0.33 | 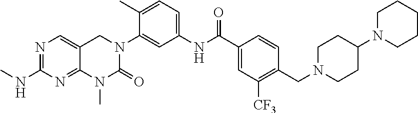 [SMILES: CNc1ncc2c(n1)N(C)C(=O)N(c1cc(NC(=O)c3ccc(CN4CCC(N5CCCCC5)CC4)c(C(F)(F)F)c3)ccc1C)C2] |
| 6 <0.0018 <0.0026 | 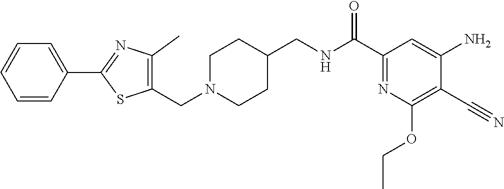 [SMILES: CCOc1nc(C(=O)NCC2CCN(Cc3sc(-c4ccccc4)nc3C)CC2)cc(N)c1C#N] |
| 7 >30 34 ± 12 | 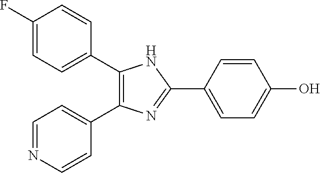 [SMILES: Oc1ccc(-c2nc(-c3ccncc3)c(-c3ccc(F)cc3)[nH]2)cc1] |
| 8 1.3 ± 0.5 >30 | 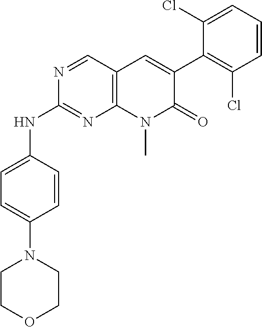 [SMILES: Cn1c(=O)c(-c2c(Cl)cccc2Cl)cc2cnc(Nc3ccc(N4CCOCC4)cc3)nc21] |
| 9 0.97 ± 0.30 1.3 ± 0.4 | 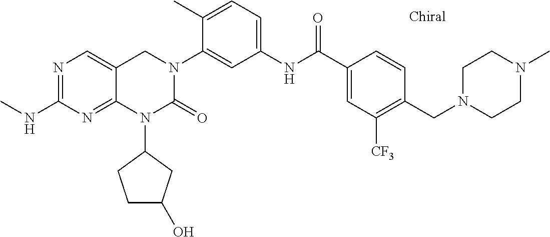 |
| 10 0.10 ± 0.03 0.14 ± 0.03 | 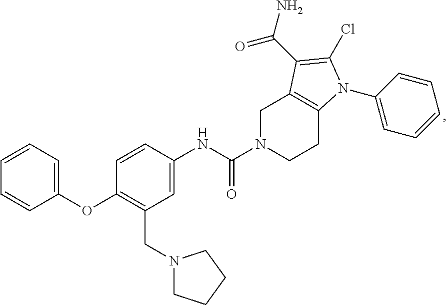 [SMILES: NC(=O)c1c2c(n(-c3ccccc3)c1Cl)CCN(C(=O)Nc1ccc(Oc3ccccc3)c(CN3CCCC3)c1)C2] |

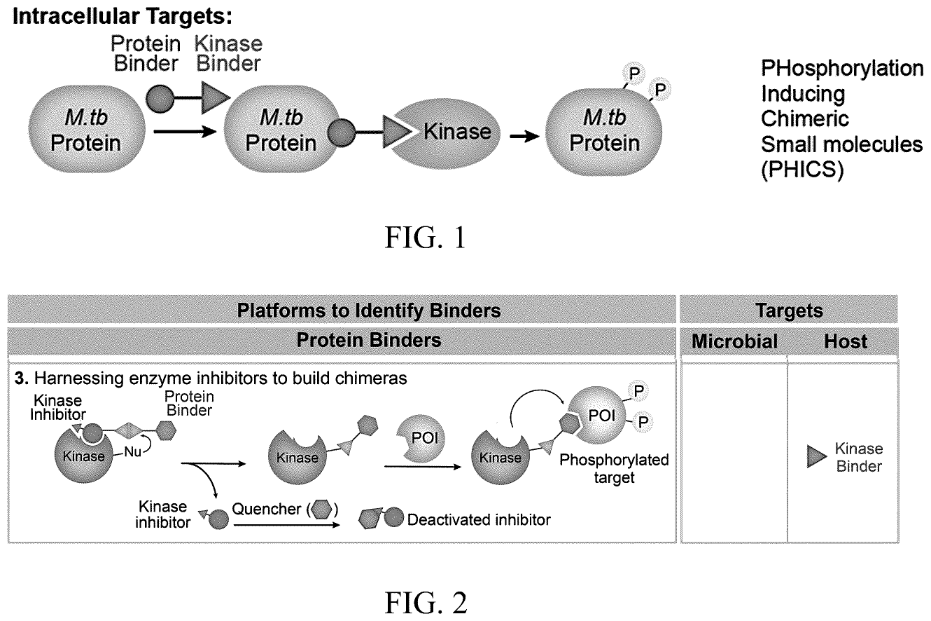

as adapted from Lombard et al. Allosteric modulation of JNK docking-site interactions with ATP-competitive inhibitors. Biochemistry. Author manuscript; available in PMC 2019 Oct. 9, FIG. 1B, incorporated herein by reference.

In an example embodiment, the JNK inhibitor is according to the formula:

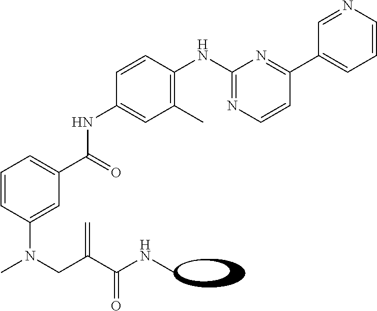 [SMILES: C=C(CN(C)c1cccc(C(=O)Nc2ccc(Nc3nccc(-c4cccnc4)n3)c(C)c2)c1)C(=O)NC]

or a derivative thereof and the oval shape represents the remaining chimeric small molecule.

### TRK Binding Moiety

The Neurotrophic Tyrosine Kinase Receptor 1 gene (NTRK1) encodes the Tropomyosin-related kinase A (TRKA) receptor tyrosine kinase. TRKA is a high affinity receptor for Nerve Growth Factor (NGF) and a member of the neurotrophin receptor family of receptor tyrosine kinases. TRKA is critical for the development and maturation of the central and peripheral nervous systems during embryogenesis. It is implicated in pain and temperature sensing in sympathetic and sensory nerves as well as memory processes in adults it is expressed in the basal forebrain. NGF-mediated dimerization actives TRKA, which induces autophosphorylation of specific tyrosine residues and transphosphorylation of additional substrates, leading to activation of the PI3K/AKT, Ras/MAPK and PLC-γ pathways. See, e.g., Ardini, E., et al. “The TPM3-NTRK1 Rearrangement Is a Recurring Event in Colorectal Carcinoma and Is Associated with Tumor Sensitivity to TRKA Kinase Inhibition.” Molecular Oncology 2014, 8 (8), 1495-1507.

In one example embodiment the protein binding moiety is a TRK binding moiety. In one example embodiment, the TRK binding moiety is an inhibitor or activator. In one example embodiment, the TRK inhibitor is any one of compounds 13-16, according to the formulas:

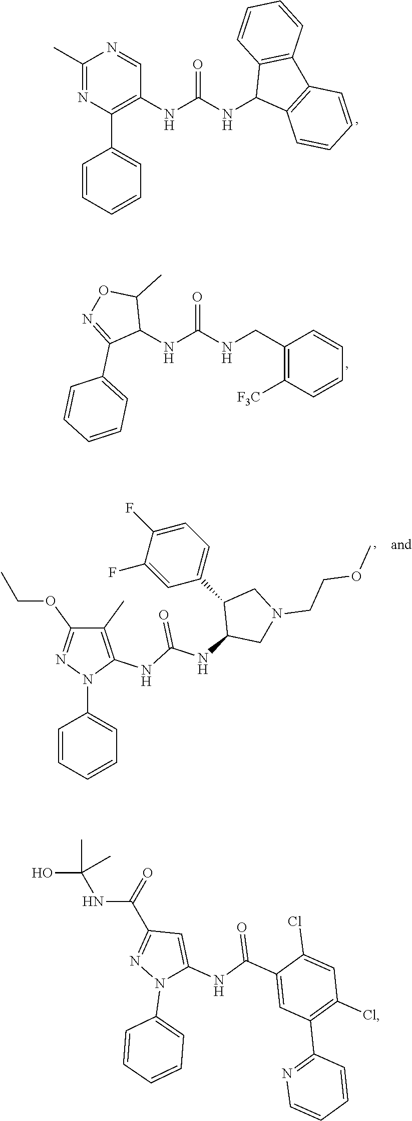 [SMILES: Cc1ncc(NC(=O)NC2c3ccccc3-c3ccccc32)c(-c2ccccc2)n1 ; CC1ON=C(c2ccccc2)C1NC(=O)NCc1ccccc1C(F)(F)F ; CCOc1nn(-c2ccccc2)c(NC(=O)N[C@@H]2CN(CCOC)C[C@H]2c2ccc(F)c(F)c2)c1C ; CC(C)(O)NC(=O)c1cc(NC(=O)c2cc(-c3ccccn3)c(Cl)cc2Cl)n(-c2ccccc2)n1]

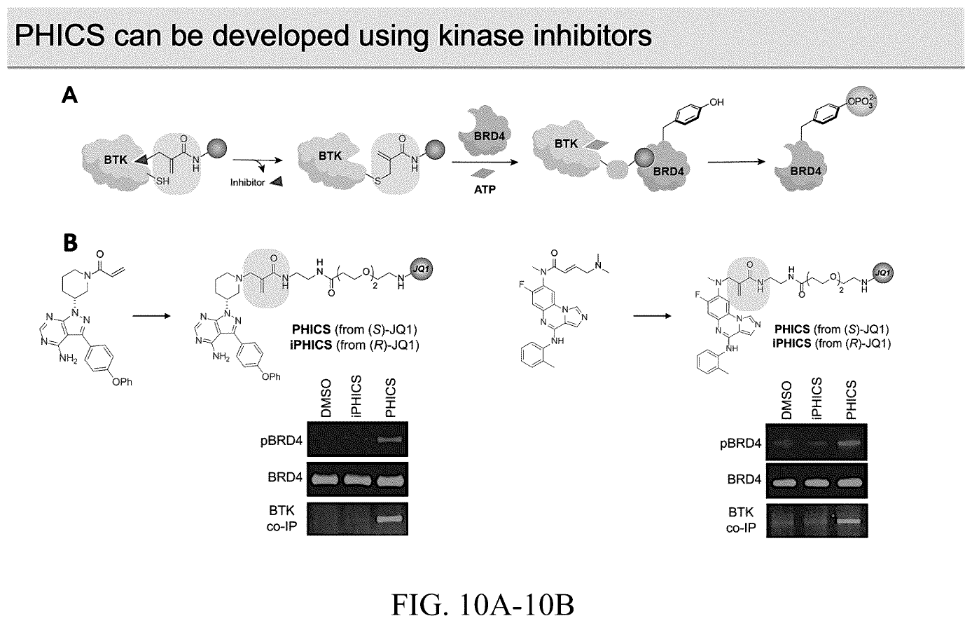

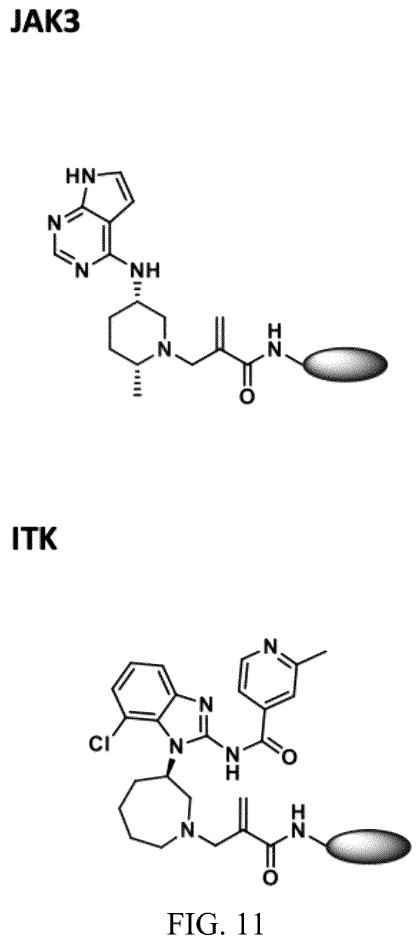

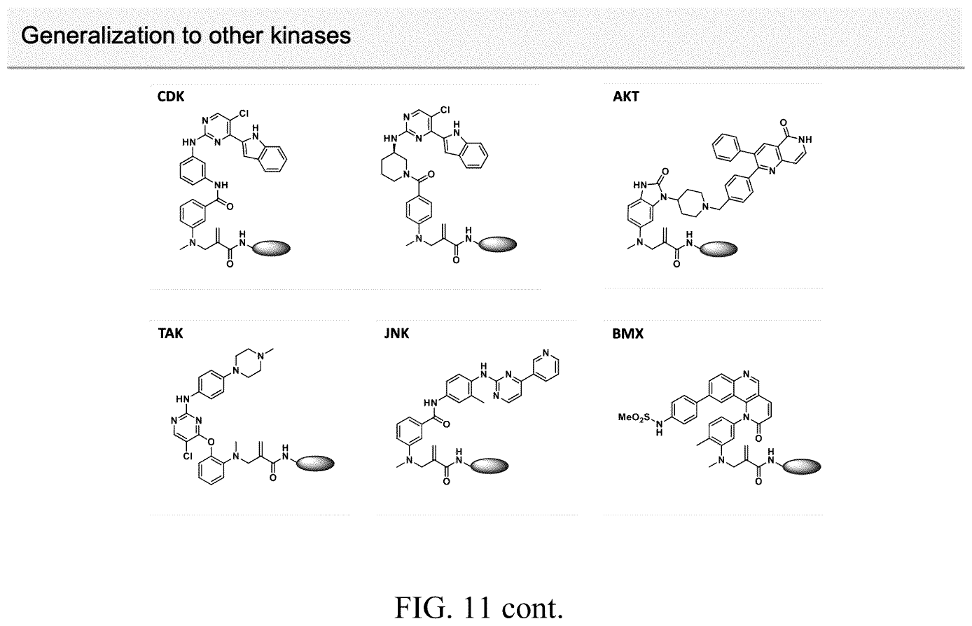

respectively. Compound 13, an allosteric inhibitor of TRKA, has an IC₅₀ of 99 nM and good selectivity over TRKB and TRKC, each of which has an IC₅₀ value of 81 mM and 25 mM respectively. In addition, Compound 15 also demonstrated good selectivity for TRKA over TRKB. Crystal structures of TRKA and Compounds 13, 15 and 16 from Patent Application No. CN103649076, in particular, FIGS. 10 and 11 are incorporated herein by reference (PDB codes: 5KMI, 5H3Q). In another example embodiment, the TRK inhibitor is any pyrrolidinyl urea or pyrrolidinyl thiourea as described in the International Patent Application WO2012158413A2, herein incorporated by reference, as well as any derivates thereof.

Additional Trk inhibitors are described in Bailey et al, Tropomyosin receptor kinase inhibitors: an updated patent review for 2010-2016, Expert Opinion on Therapeutic Patents doi: 10.1080/13543776.2017.1297797, and Bailey et al., (2020) Tropomyosin receptor kinase inhibitors: an updated patent review for 2016-2019, Expert Opinion on Therapeutic Patents, 30:5, 325-339, DOI: 10.1080/13543776.2020; both incorporated herein by reference in their entirety.

### PDGFR Binding Moiety

Platelet-derived growth factor (PDGF) system includes two receptors: PDGFRA and PDGFRB and four ligands: PDGFA; PDGFB; PDGFC; and PDGFD. Ligand binding induces receptor dimerization, enabling autophosphorylation of specific tyrosine residues and subsequent recruitment of a variety of signal transduction molecules. PDGFR regulates normal cellular growth and differentiation, and expression of activated PDGFR promotes oncogenic transformation, see e.g. McDermott, U., et al. “Ligand-Dependent Platelet-Derived Growth Factor Receptor (PDGFR)-α Activation Sensitizes Rare Lung Cancer and Sarcoma Cells to PDGFR Kinase Inhibitors. Cancer Res 2009, 69 (9), 3937-3946. In one example embodiment, the kinase binding moiety is a PDGFR targeting molecule. In one example embodiment, the PDGFR binding moiety is an inhibitor or activator. In an example embodiment, the PDGFR targeting molecule is imatinib, nilotinib, or dasatinib.

### IDH Binding Moiety

Isocitrate dehydrogenases (IDHs) participate in cellular metabolism by converting isocitrate to α-ketoglutarate in a NADP⁺-dependent manner. There are three isozymes-IDH1, IDH2, IDH3-which function in distinct subcellular compartments. Mutation of IDHs may result in production of 2-hydroxyglutarate instead of a normal product, leading to various types of cancers. See e.g., Jiang, B.; et al., IDH1 Mutation Promotes Tumorigenesis by Inhibiting JNK Activation and Apoptosis Induced by Serum Starvation. Cell Reports, 2017, 19, 389-400 and Malarz, K.; et al., The Landscape of the Anti-Kinase Activity of the IDH1 Inhibitors. Cancers, 2020, 12, 536.

In example embodiments, the kinase binding moiety is a IDH targeting molecule. In an example embodiment, the IDH targeting molecule is an inhibitor or activator. In an example embodiment, the IDH inhibitor is Enasidenib, Ivosidenib, or any derivatives thereof.

### RET Binding Moiety

Rearranged during transfection (RET) oncogenic gene fusions encodes a receptor tyrosine kinase for glial cell line-derived neurotrophic factor (GDNF)-family ligands. RET has been shown to play role in signal transduction for kidney development and enteric nervous system. See e.g., Arighi, E.; Borrello, M. G.; Sariola, H. RET Tyrosine Kinase Signaling in Development and Cancer. Cytokine &amp; Growth Factor Reviews, 2005, 16, 441-467. In example embodiments, the kinase binding moiety is a RET targeting molecule. In an example embodiment, the RET targeting molecule is an inhibitor or activator. In an example embodiment, the RET inhibitor is Pralsetinib or any derivatives thereof.

### ITK Binding Moiety

Interleukin-2-inducible T-cell kinase (ITK) also known as tyrosine-protein kinase (TSK) is a member of the TEC family of kinases generally expressed in T cells and NK cells. ITK mediates signal transduction in T cells and NK cells initiated by T cell receptors and Fc receptors. See e.g., Zhong, Y.; et al., Targeting Interleukin-2 Inducible T-Cell Kinase (ITK) in T-Cell Related Diseases. Postdoc Journal, 2014, 2.

In example embodiments, the kinase binding moiety is a ITK targeting molecule. In an example embodiment, the ITK targeting molecule is an inhibitor or activator. In an example embodiment, the ITK inhibitor is according to the formula:

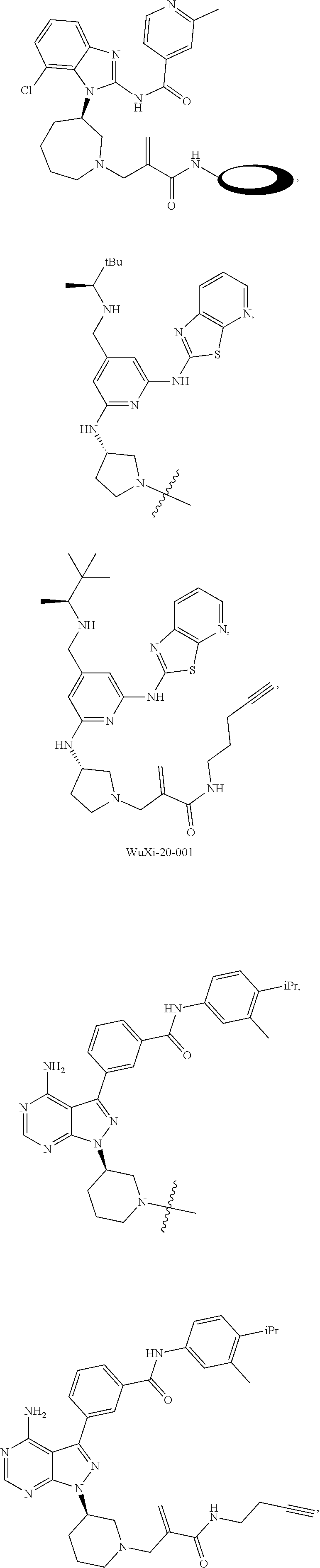

or a derivative thereof and the oval shape represents the remaining chimeric small molecule and the yne-group represents the bonding point for the remaining chimeric small molecule.

### TAK Binding Moiety

Transforming growth factor β-activated kinase 1 (TAK1) is a member of the MAPK family and functions as a signaling intermediate in the tumor necrosis factor (TNF), interleukin 1, and Toll-like receptor signaling pathways. TAK1 transmits the receptor complex signal to the downstream MAPKs and NF-κB pathways. TAK1 initiates a signaling cascade by associating with the TNF receptor complex through TNF receptor-associated factor 2/5 (TRAF2/5) and the kinase receptor-interacting protein 1 (RIP1). See e.g., Broglie, P. et al., J. Transforming Growth Factor β-Activated Kinase 1 (TAK1) Kinase Adaptor, TAK1-Binding Protein 2, Plays Dual Roles in TAK1 Signaling by Recruiting Both an Activator and an Inhibitor of TAK1 Kinase in Tumor Necrosis Factor Signaling Pathway. Journal of Biological Chemistry, 2010, 285, 2333-2339

In example embodiments, the kinase binding moiety is a TAK1 targeting molecule. In an example embodiment, the TAK1 targeting molecule is an inhibitor or activator. In an example embodiment, the TAK1 inhibitor is according to the formula:

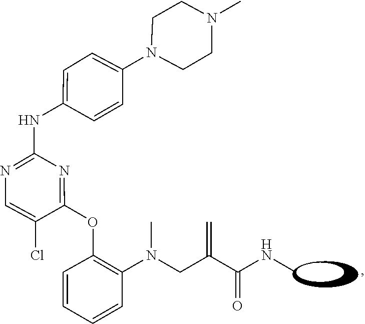 [SMILES: C=C(CN(C)c1ccccc1Oc1nc(Nc2ccc(N3CCN(C)CC3)cc2)ncc1Cl)C(=O)NC]

or a derivative thereof and the oval shape represents the remaining chimeric small molecule.

### BMX Binding Moiety

Bone marrow kinase on chromosome X (BMX) belongs to the family of tyrosine kinase expressed in hepatocellular carcinoma (TEC) family of nonreceptor tyrosine kinases. BMX is expressed in the endocardium cells of the heart, endothelial cells of the heart, and hematopoietic cells of the myeloid lineage such as granulocytes and monocytes. BMX may function in cellular differentiation, motility, and survival. See e.g., Gottar-Guillier, M.; Dodeller, F.; Huesken, D.; Iourgenko, V.; Mickanin, C.; Labow, M.; Gaveriaux, S.; Kinzel, B.; Mueller, M.; Alitalo, K.; Littlewood-Evans, A.; Cenni, B. The Tyrosine Kinase BMX Is an Essential Mediator of Inflammatory Arthritis in a Kinase-Independent Manner. The Journal of Immunology, 2011, 186, 6014-6023.

In example embodiments, the kinase binding moiety is a BMX targeting molecule. In an example embodiment, the BMX targeting molecule is an inhibitor or activator. In an example embodiment, the BMX inhibitor is according to the formula:

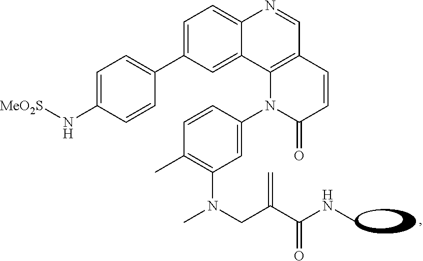 [SMILES: C=C(CN(C)c1cc(-n2c(=O)ccc3cnc4ccc(-c5ccc(NS(C)(=O)=O)cc5)cc4c32)ccc1C)C(=O)NC]

or a derivative thereof and the oval shape represents the remaining chimeric small molecule.

### LIMK Binding Moiety

LIM kinases (LIMK) comprise of two closely related kinases, LIMK1 and LIMK2. LIMK family members' structure comprise of two amino-terminal LIM domains, PDZ region, proline/serine (P/S)-rich regions, and a kinase domain. These components form a signaling domain, which controls cytoskeleton dynamics through the phosphorylation of the cofilin family proteins and are downstream effectors of several signalization pathways. See e.g., Prunier C, Prudent R, Kapur R, Sadoul K, Lafanechère L. LIM kinases: cofilin and beyond. Oncotarget. 2017 Jun. 20; 8(25):41749-41763.

In example embodiments, the kinase binding moiety is a LIMK targeting molecule. In an example embodiment, the LIMK targeting molecule is an inhibitor or activator. In an example embodiment, the LIMK inhibitor is according to the formula:

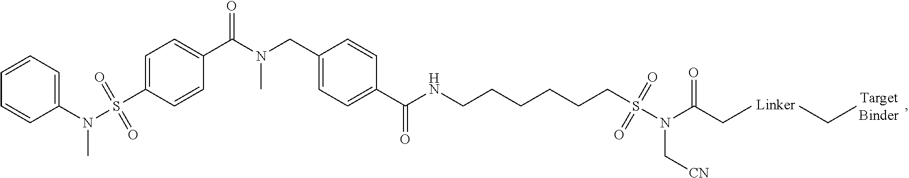

or a derivative thereof and the “Linker” and “Target Binder” represents the remaining chimeric small molecule.

### IRE Binding Moiety

serine/threonine-protein kinase/endoribonuclease inositol-requiring enzyme 1 a (IRE) is an ER transmembrane protein containing two enzymatic activities, a kinase and an endoribonuclease (RNase). During ER stress the ER luminal domain becomes oligomerized and the ER protein unfolds. Oligomerization causes rearrangement of the two enzymatic domains resulting in trans-autophosphorylation. The RNase cleaves XBP1 mRNA to release an intron. The open reading is then shifted after re-ligation of the cleaved XBP1 mRNA. A transcription factor called XBP1s is produced by the translated spliced XBP1 mRNA. ER protein folding and capacity and quality control is enhanced by a XBP1s protein. Therefore, IRE promotes adaptation of the ER.

In example embodiments, the kinase binding moiety is a IRE targeting molecule. In an example embodiment, the IRE targeting molecule is an inhibitor or activator. In an example embodiment, the IRE inhibitor is according to the formula:

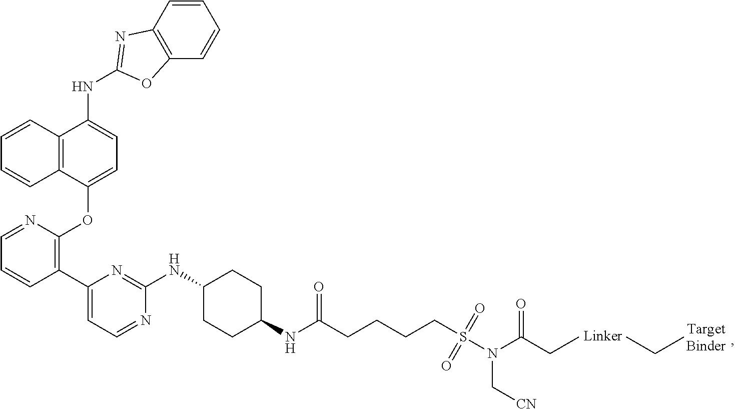

or a derivative thereof and the “Linker” and “Target Binder” represents the remaining chimeric small molecule.

### Additional Kinase Binding Moieties

In an example embodiment, the kinase binding moiety is of the formula:

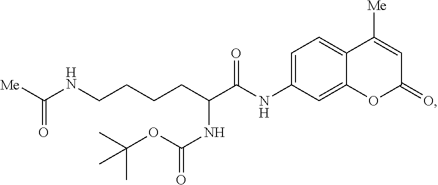 [SMILES: CC(=O)NCCCCC(NC(=O)OC(C)(C)C)C(=O)Nc1ccc2c(C)cc(=O)oc2c1]

or a derivative thereof and the remaining chimeric small molecule bonds to the methyl group on the far left.

### Target Substrates

The target substrate may be a natural substrate of the enzyme bound by the modifying polypeptide binding moieties above. However, the target binding moieties discussed below, may also be used to direct the enzyme to modify a non-natural or neo-substrate for modifying polypeptide. Target substrates may comprise polypeptides, a nucleic acid, polynucleotides, lipids, and oligosaccharides. The target binding moiety may be chosen for a specific substrate of interest, which may be located in different localization sites of the cell, e.g., nucleus, cytoplasm, mitochondria, cell surface.

### Target Binding Moiety

The target binding moiety equips the chimeric small molecule with a mechanism to bind or associate with a target, including the target substrates noted above. The target binding moiety of the chimeric small molecule binds the target substrate and brings the target substrate into proximity with a modifying polypeptide via the modifying polypeptide binding moiety or by virtue of the target binding moiety bound or labeled on the modifying polypeptide. The reaction can allow the protein to modify a larger number of substrates, non-natural target substrates of the protein, and to increase the kinetics/efficiency of such substrate modifications. For that purpose, the target binding moiety should be capable of binding the desired substrate of interest and capable of being linked to a modifying polypeptide binding moiety via a linker to allow for modification of the substrate.

### Polynucleotide Binding Moieties

In one example embodiment, the target binding moiety binds polynucleotides. Example polynucleotide binding moieties include small molecules. Small molecules that target polynucleotides include groove binders and intercalators, see e.g. Wang M., et al. “Recent Advances in Developing Small Molecules Targeting Nucleic Acid.” IJMS 2016, 17 (6), 779 and Warner K. D., et al. “Principles for Targeting RNA with Drug-like Small Molecules.” Nat Rev Drug Discov 2018, 17 (8), 547-558, herein incorporated by reference. Additional example polynucleotide binding moieties include polynucleotide binding proteins. Polynucleotide binding proteins can be identified from nucleotide-binding folds in the proteins, such as the Rossmann-type (see, e.g. Kleiger et al., J. of Mol. Biol. 323:69-76) and the P-loop containing nucleotide hydrolase folds (see, e.g., Saraste et al., Trends in Bio Sci, 15:430-434). Chauhan et al. has developed methods for the identification of ATP and GTP binding residues and Ansari et al. has designed a method specifically for NAD. Parca et al. (2012), identified nucleotide-binding sites in protein structures, and include nucleotides bound by the protein, protein name and name of organism in Table S1 of DOI: 10.1371/journal.pone.0050240, incorporated herein by reference. Accordingly, nucleotide binding moieties are known in the art and can be identified by one of skill in the art for use as a target binding moiety in the present compositions.

### Oligosaccharide Binding Moieties

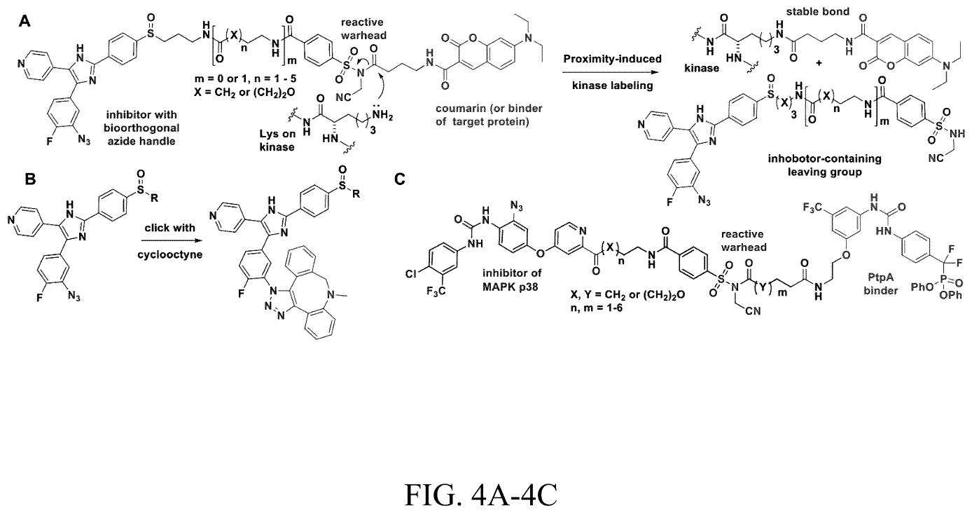

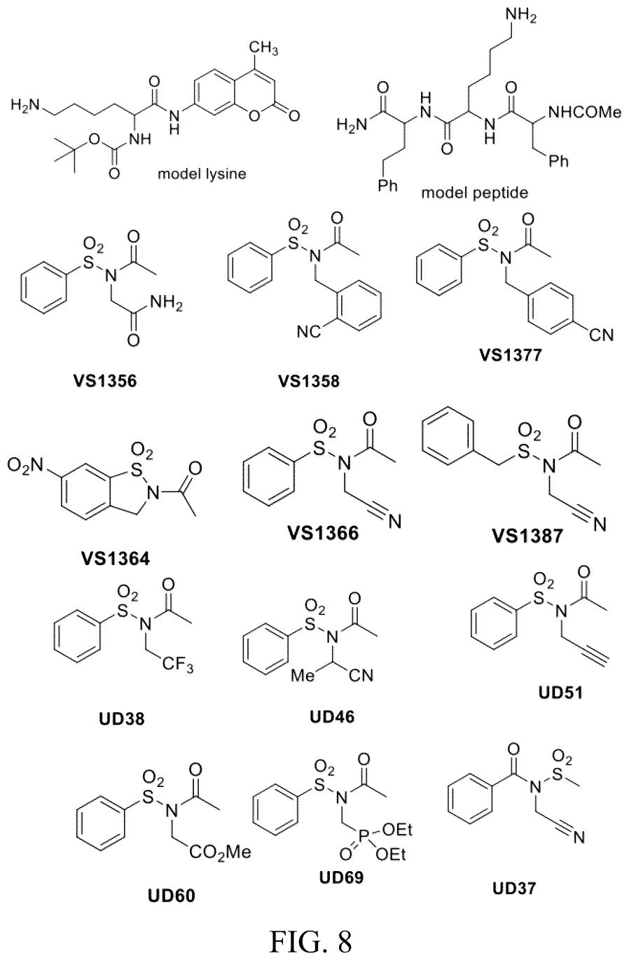

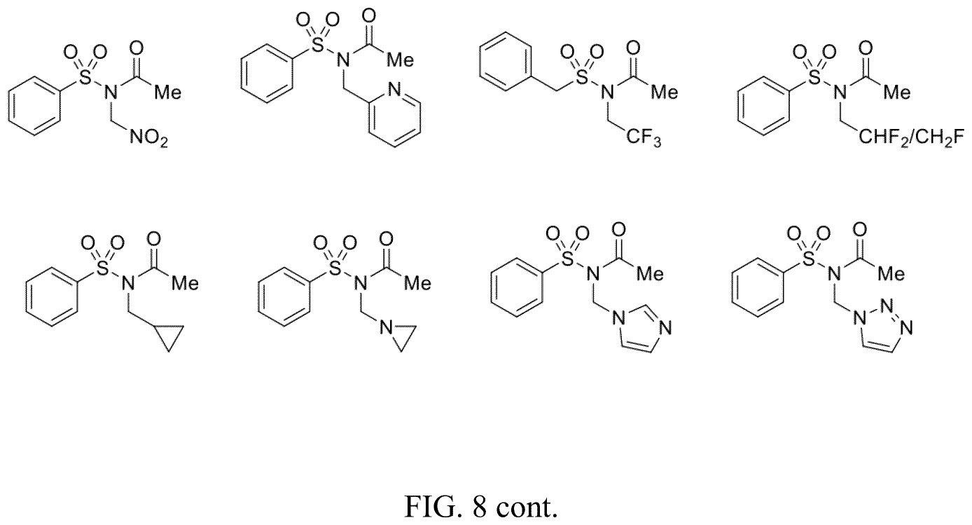

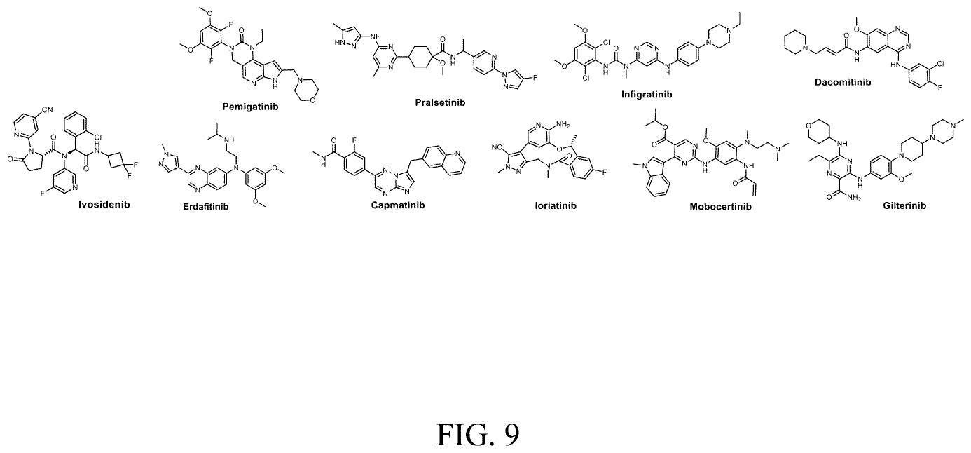

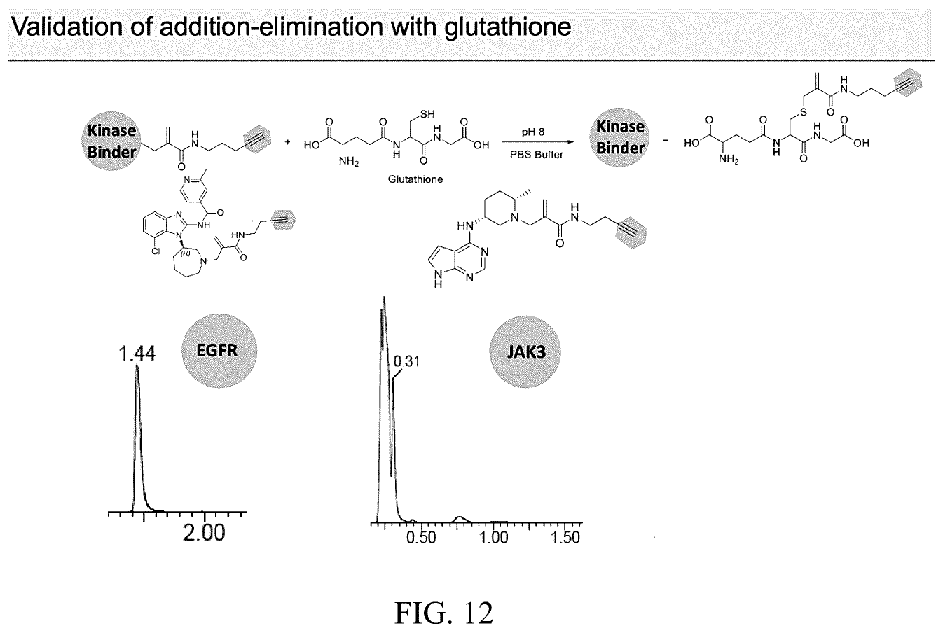

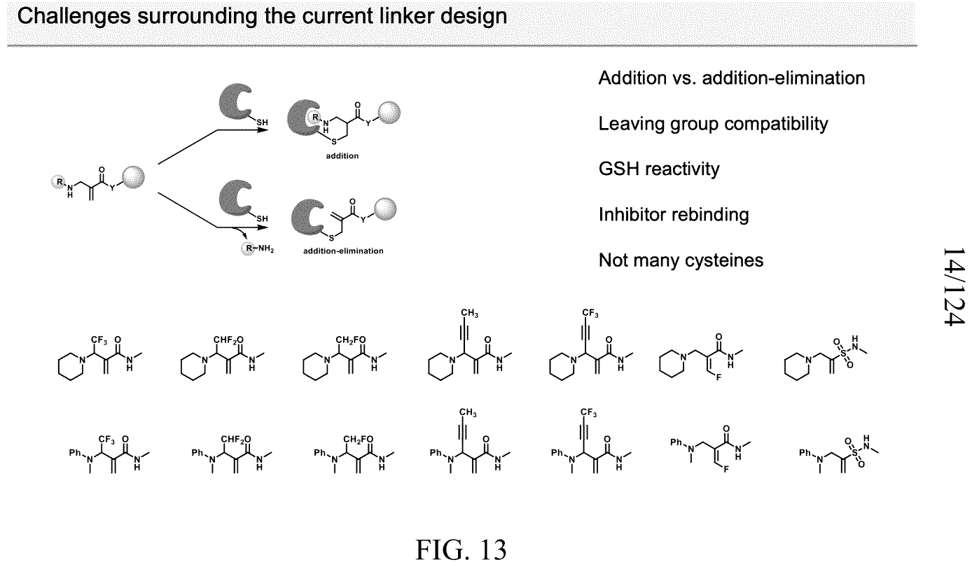

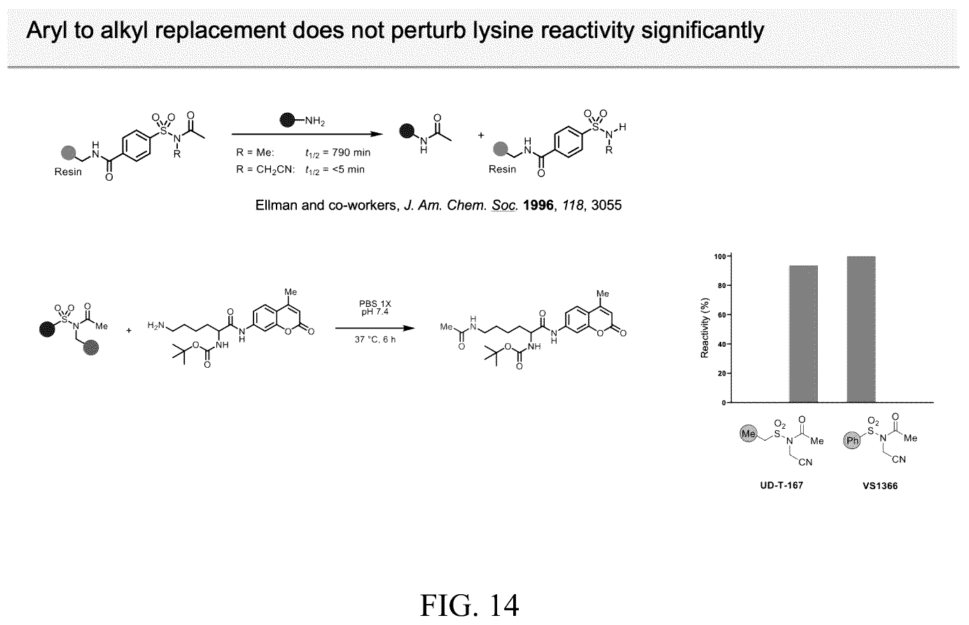

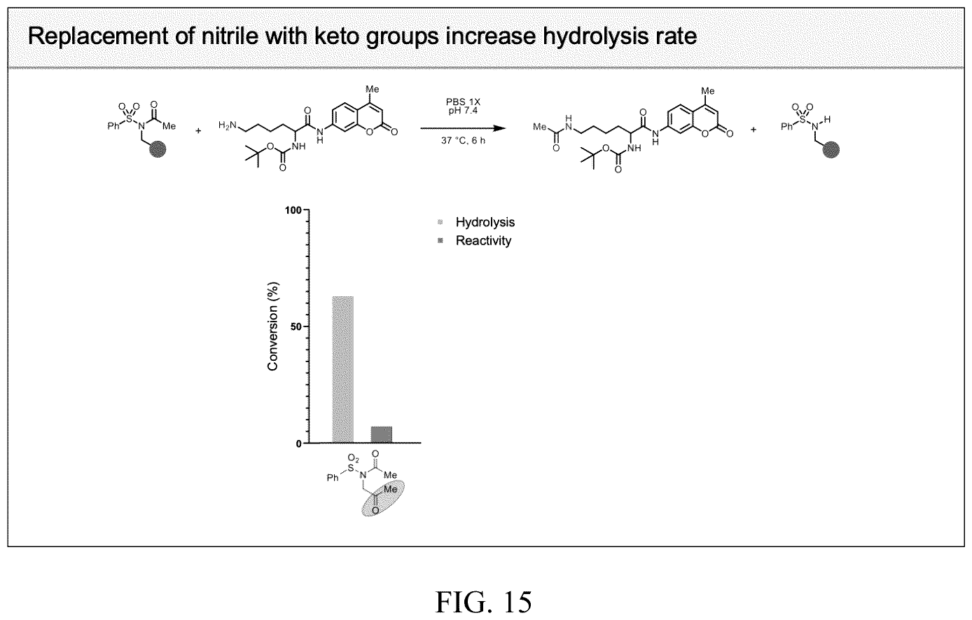

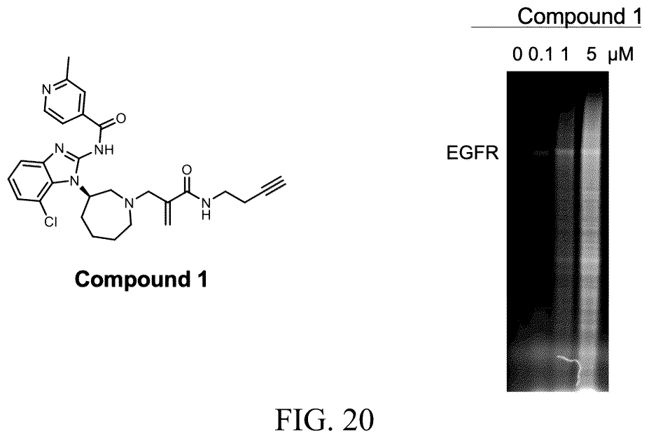

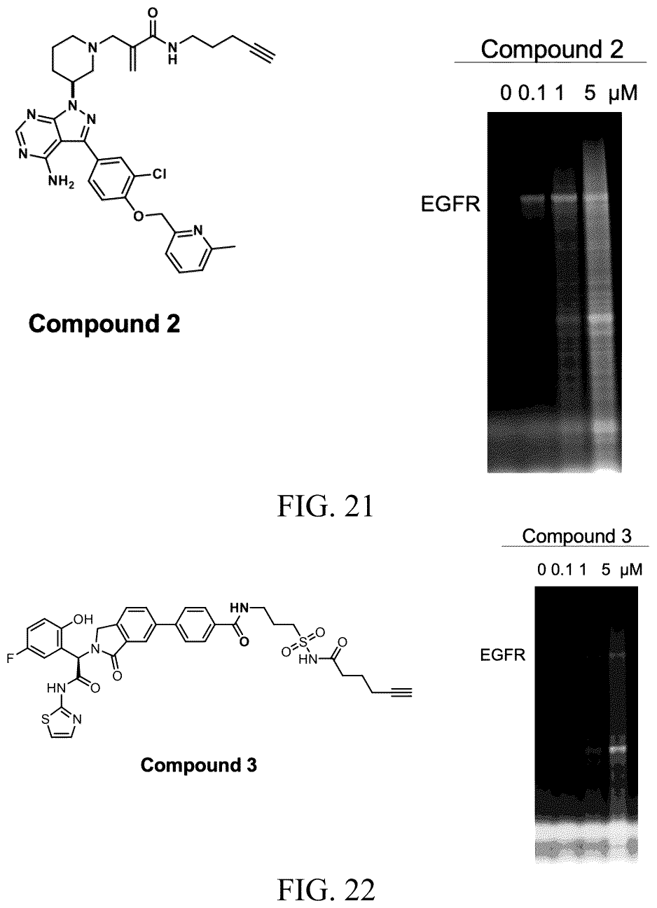

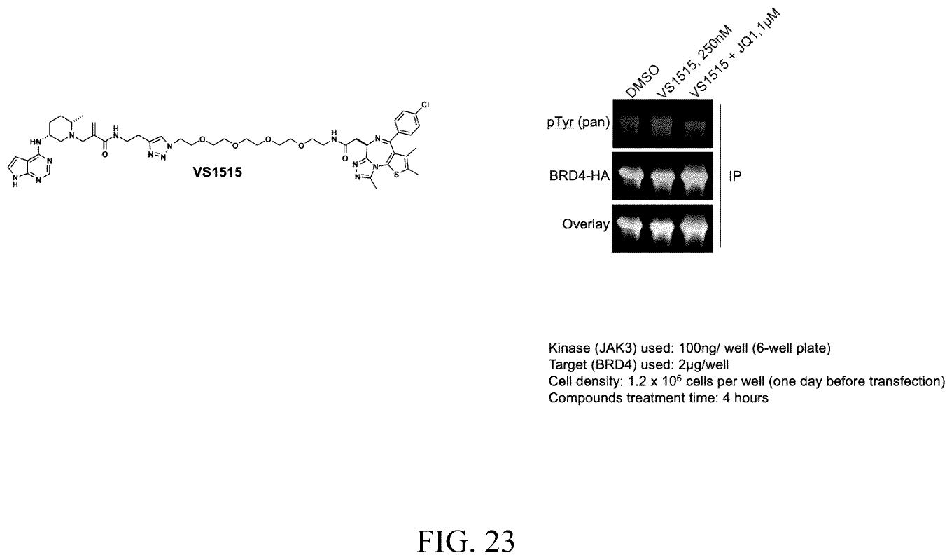

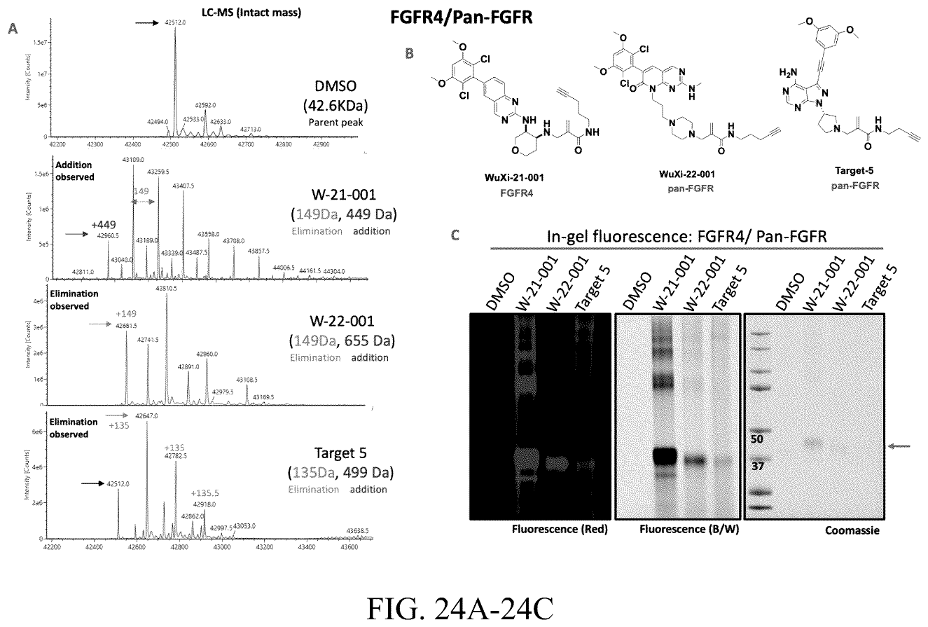

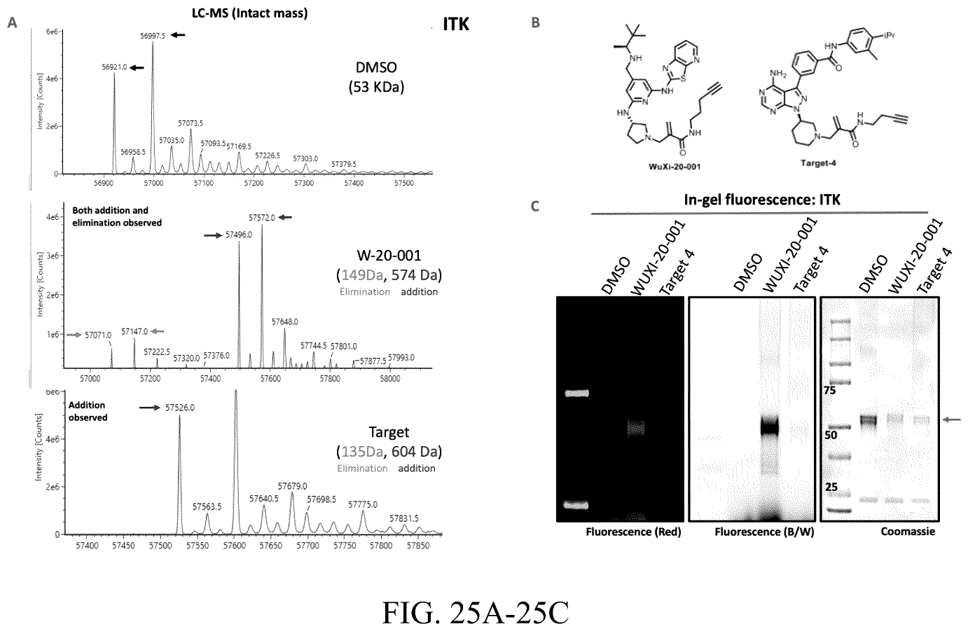

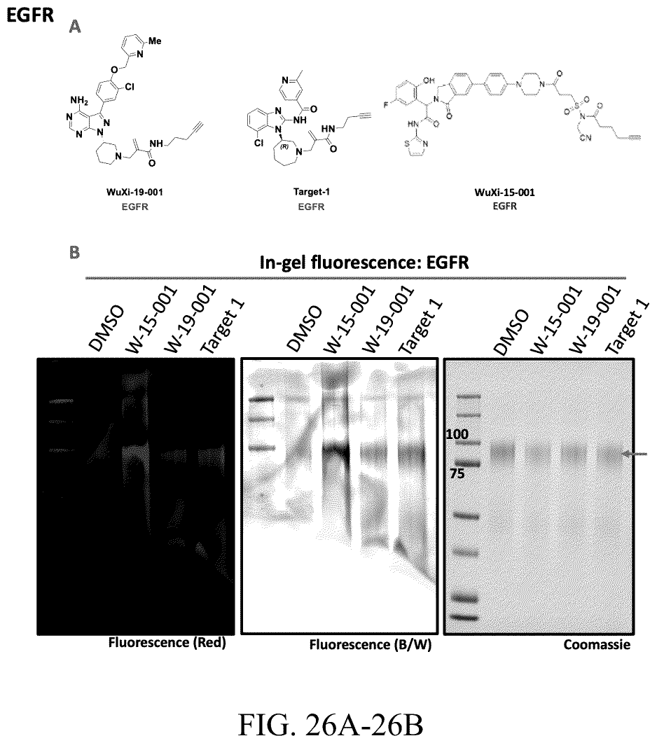

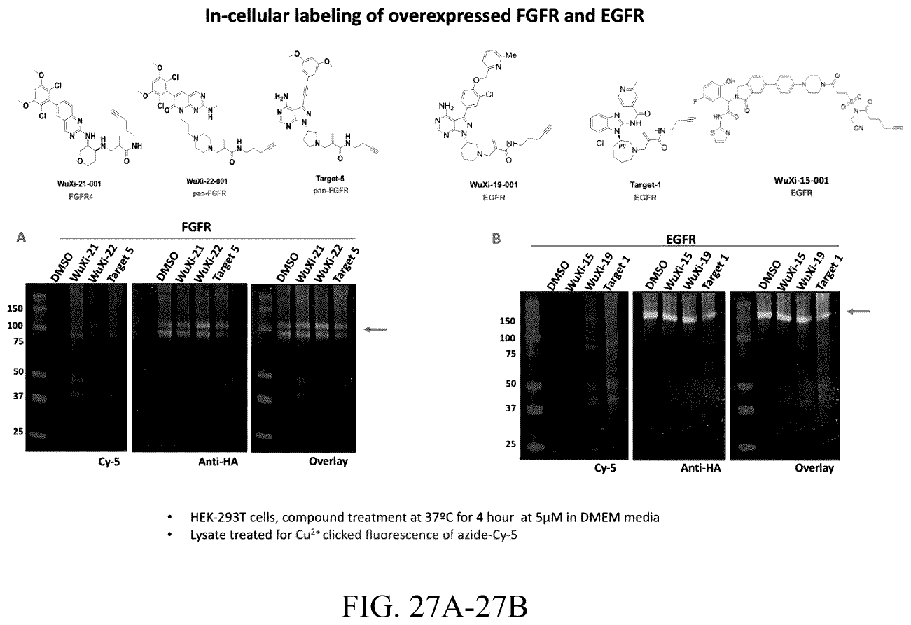

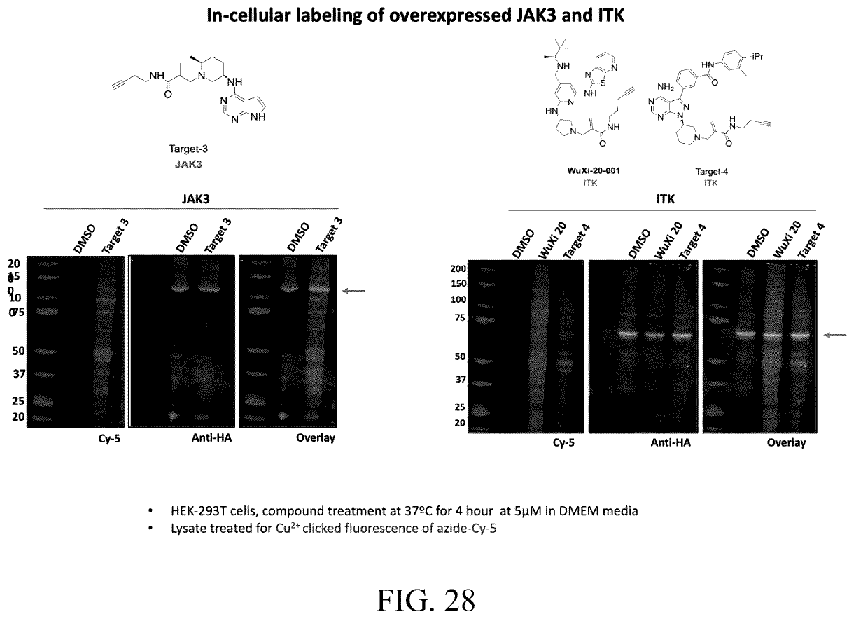

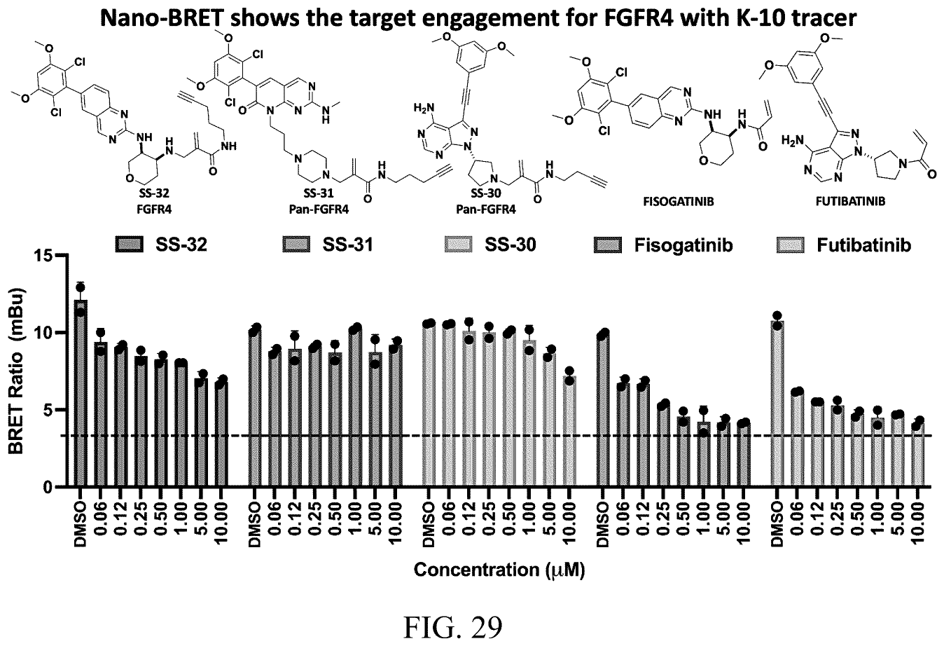

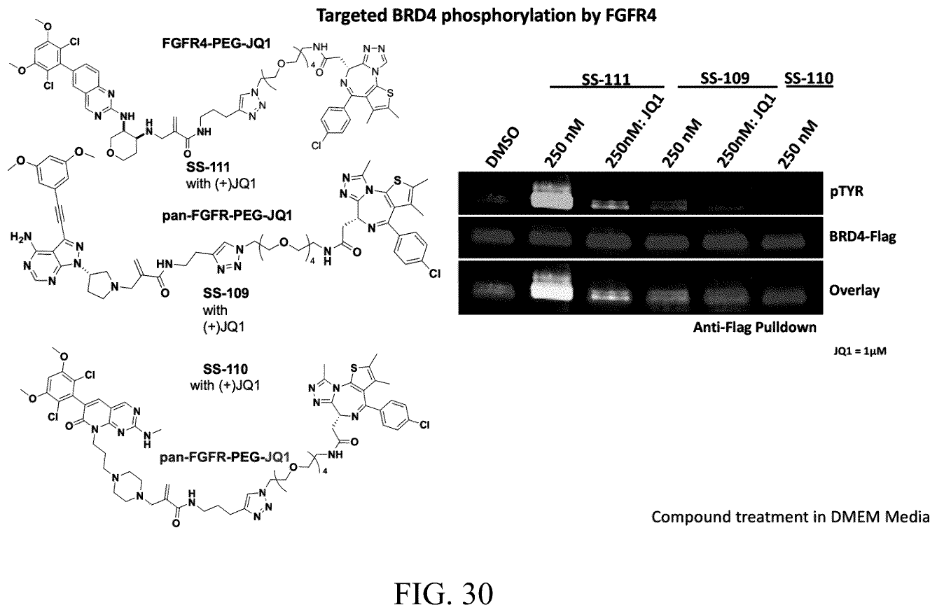

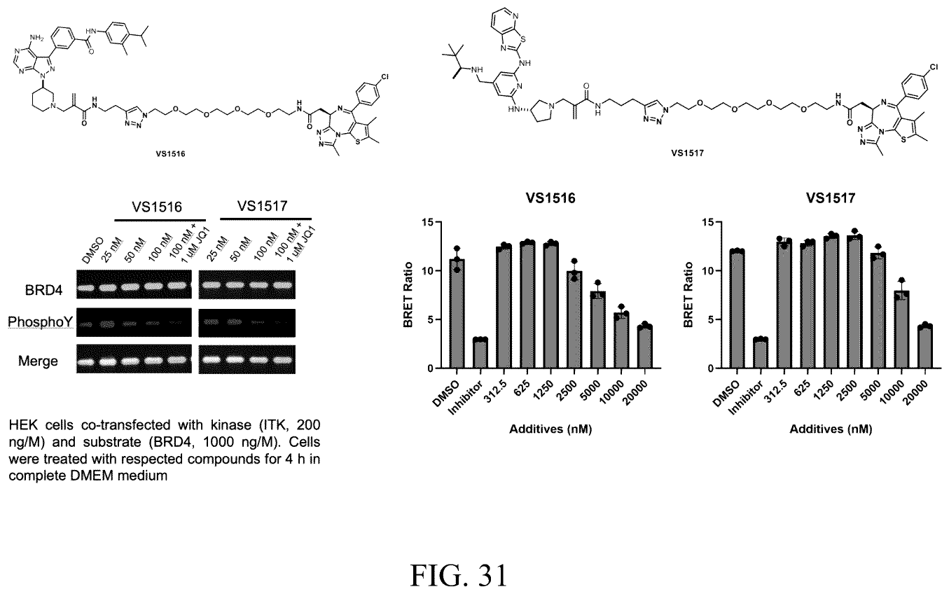

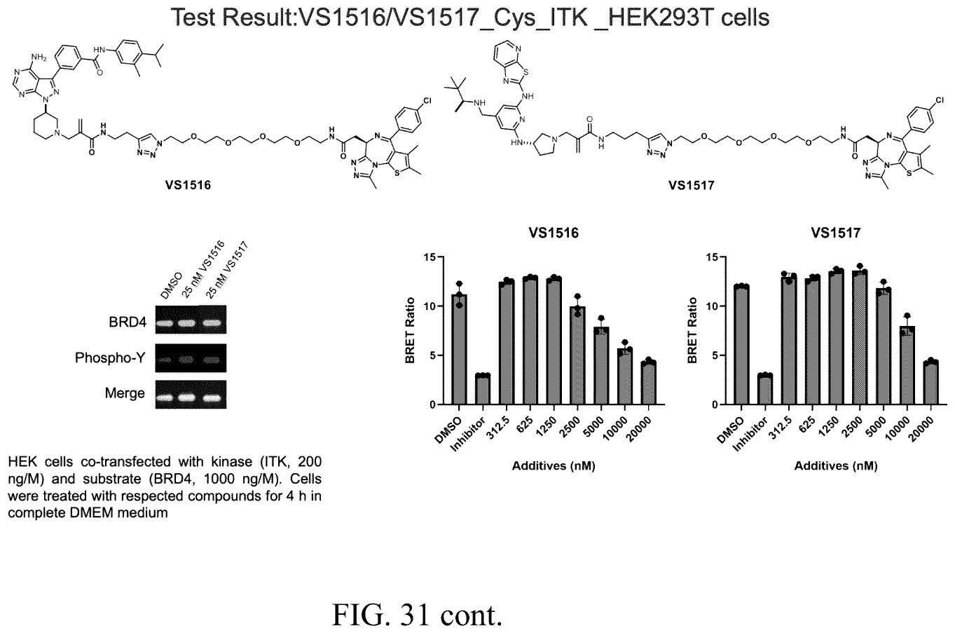

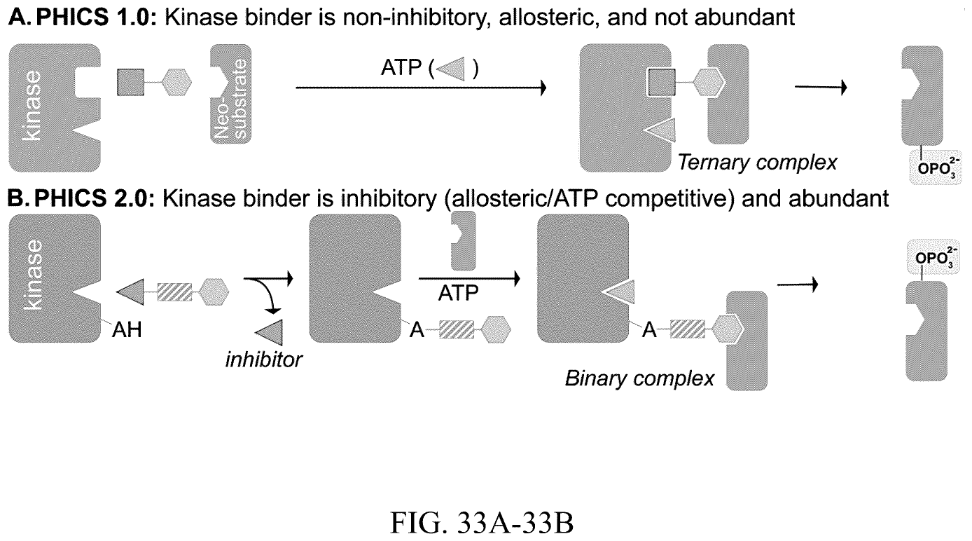

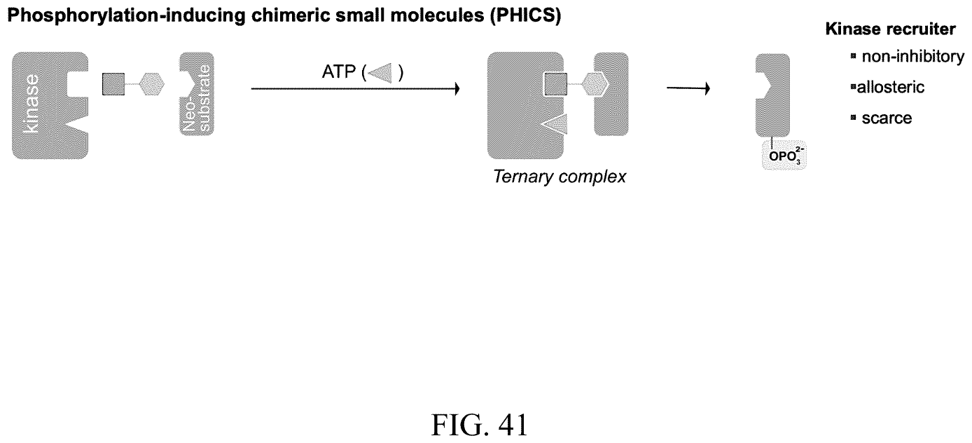

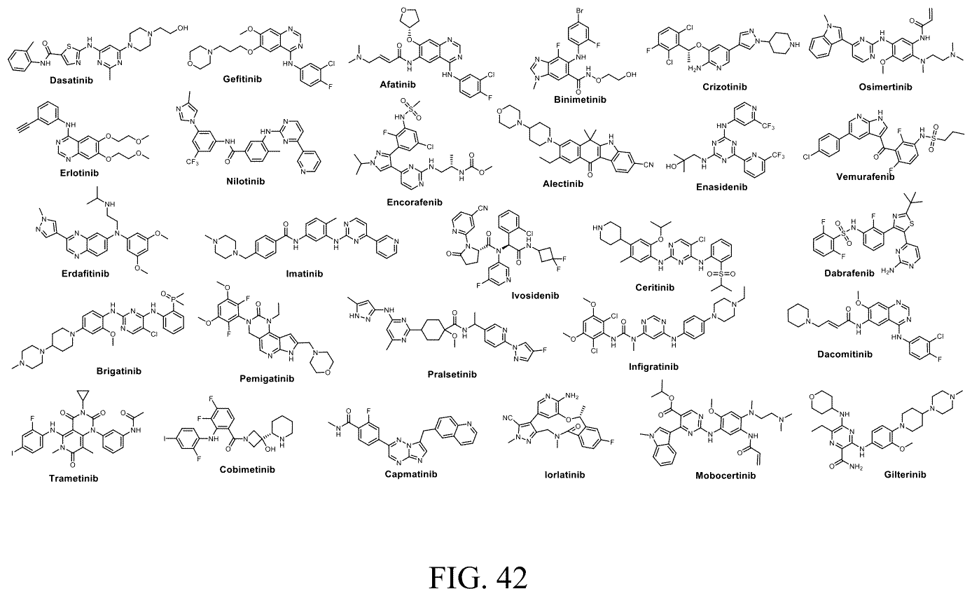

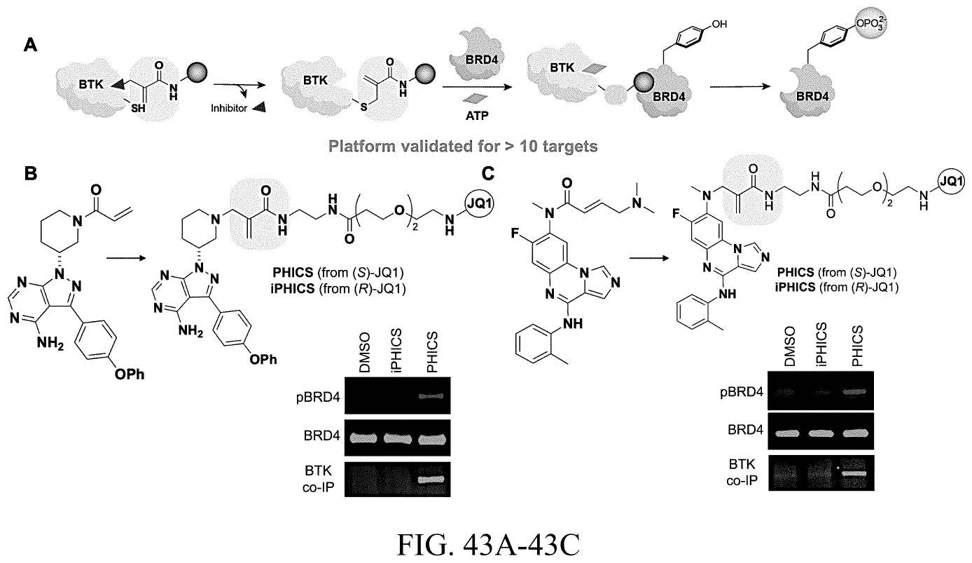

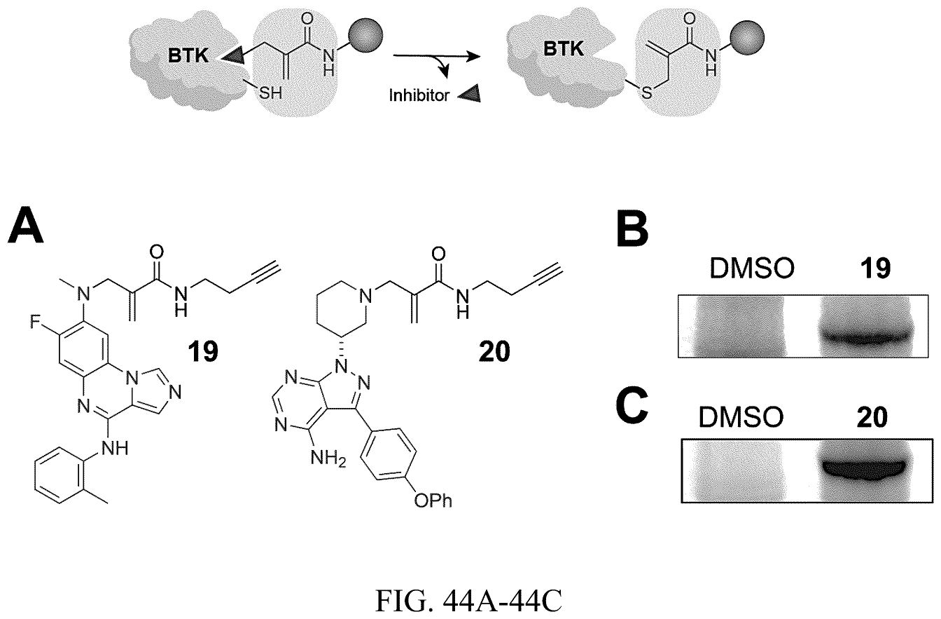

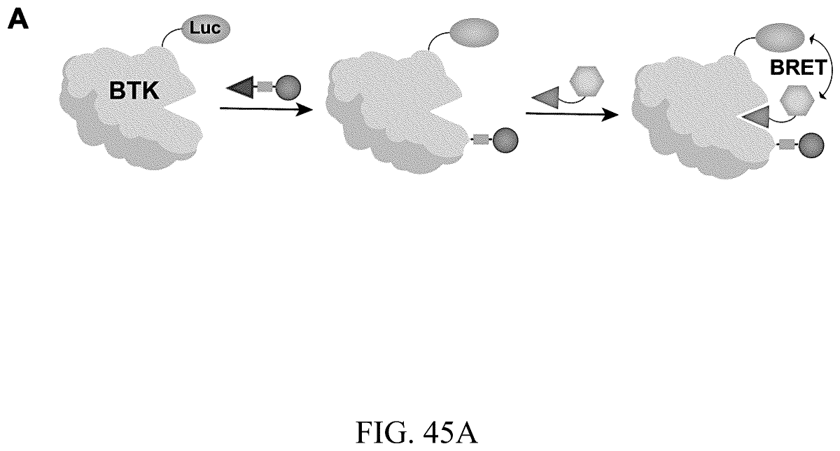

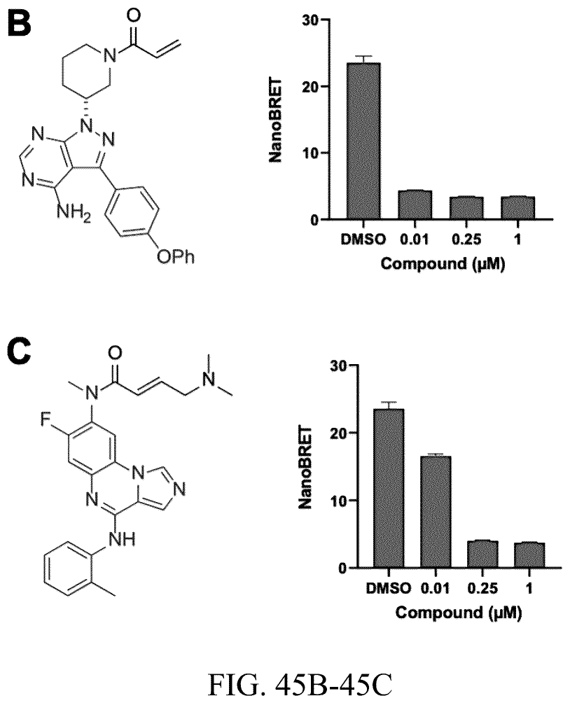

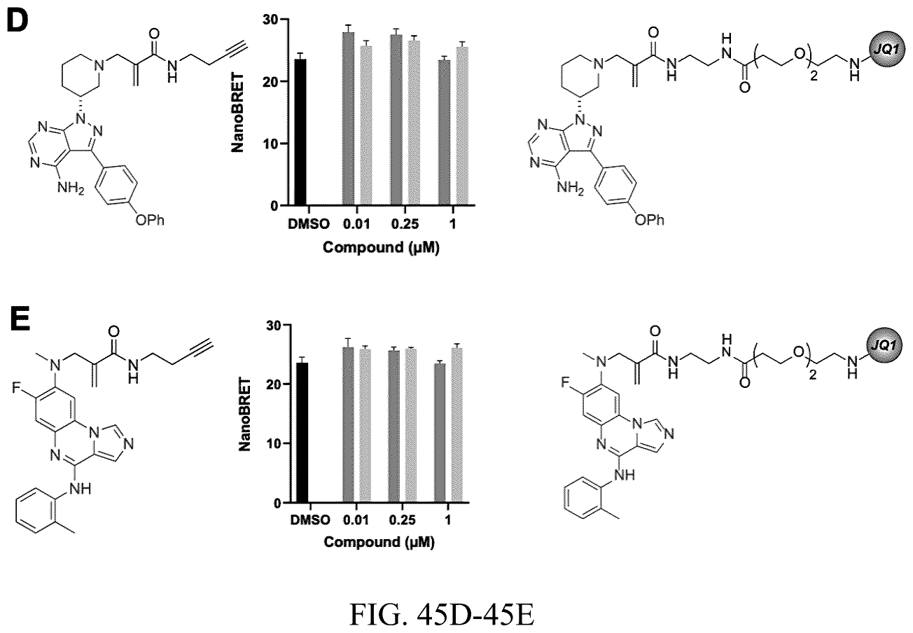

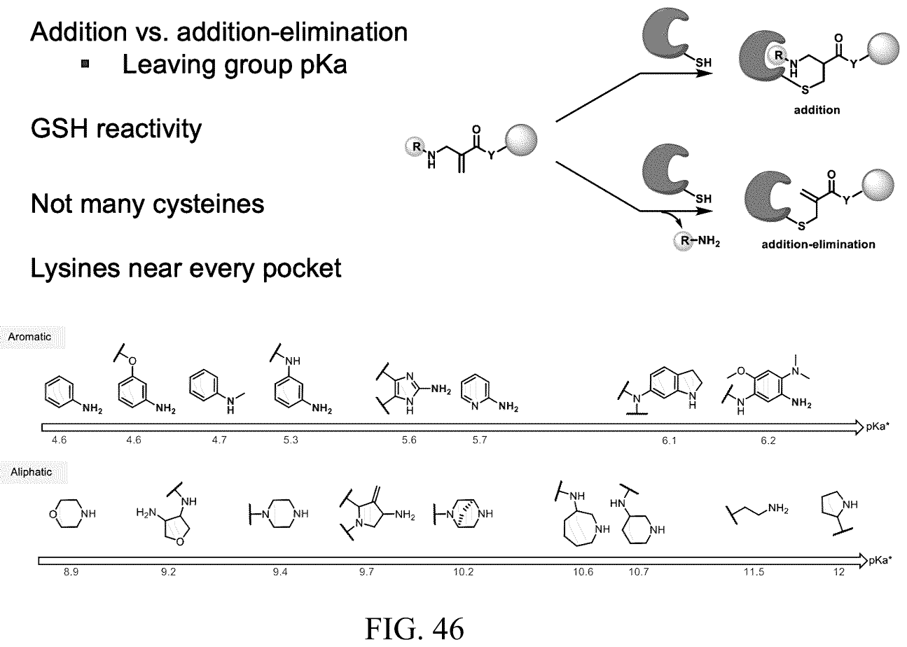

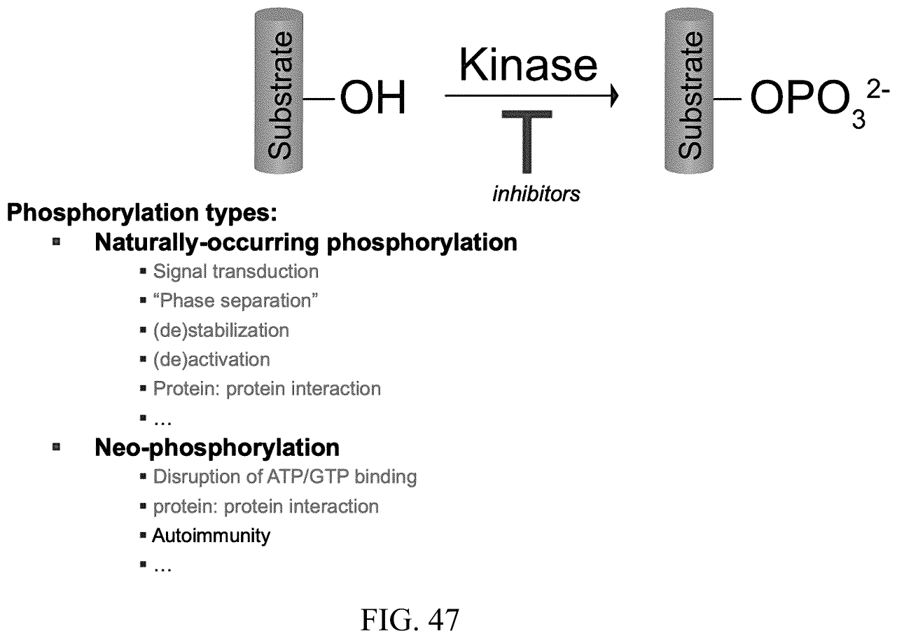

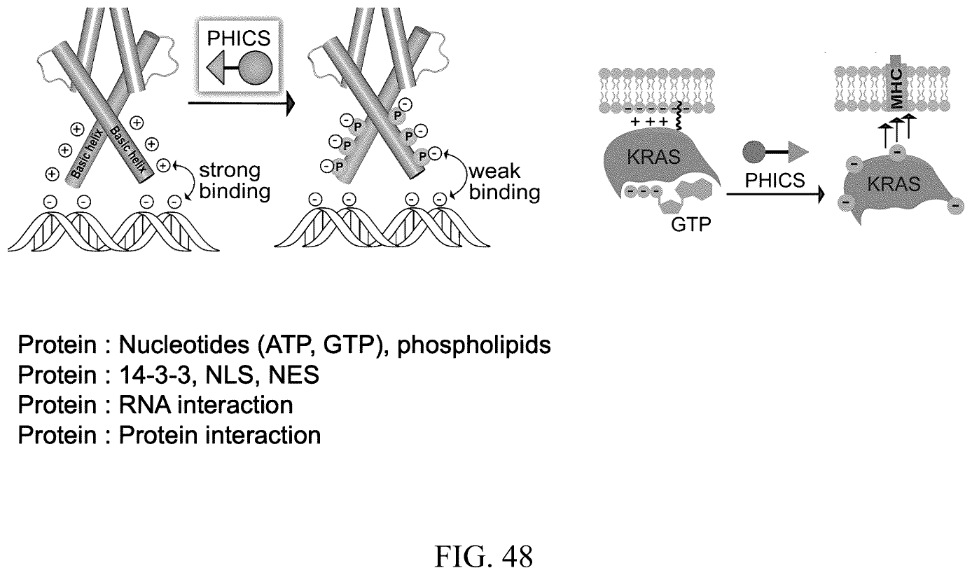

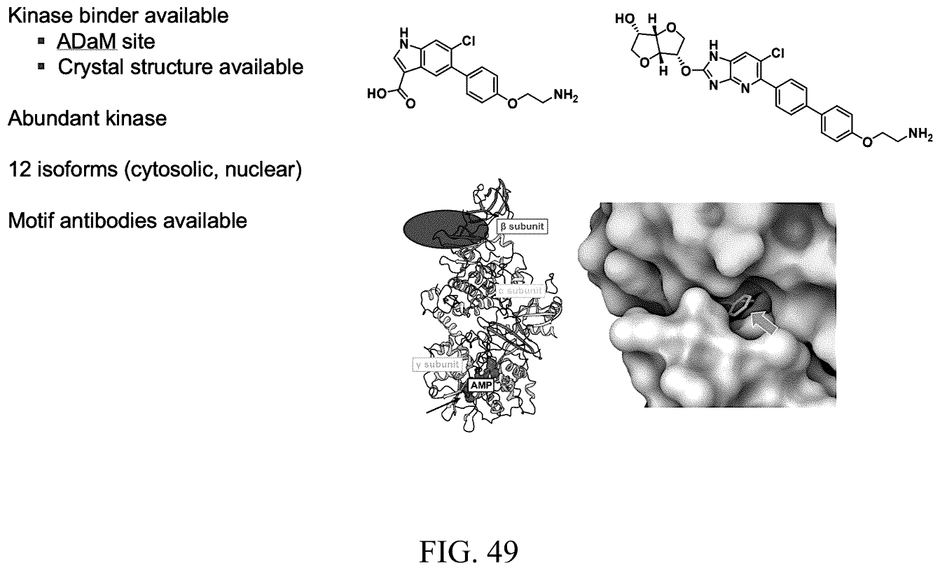

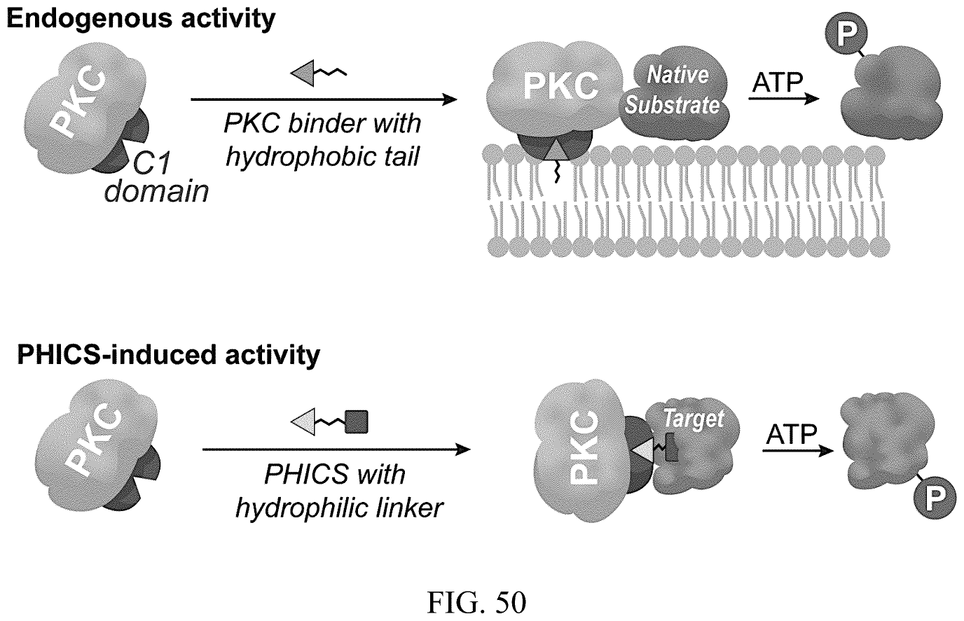

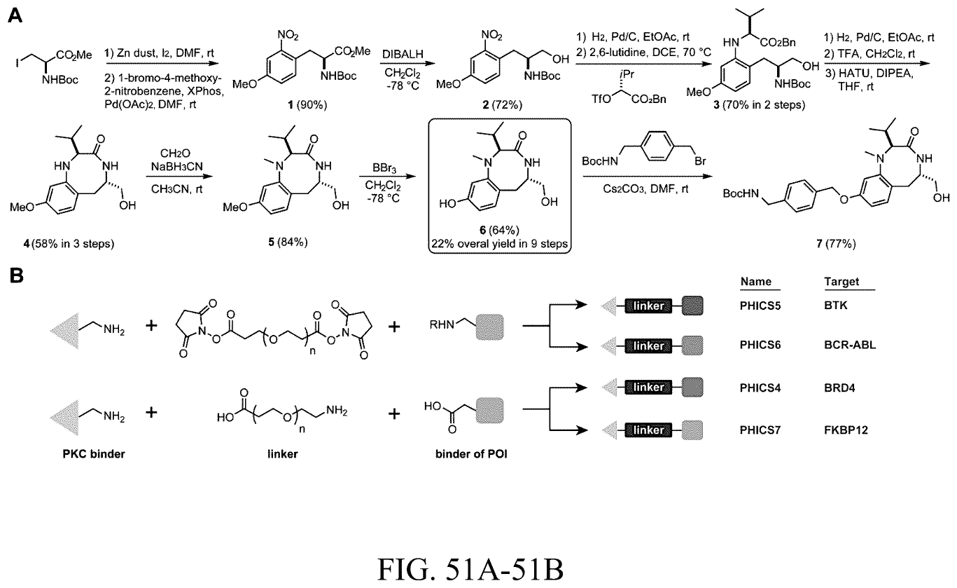

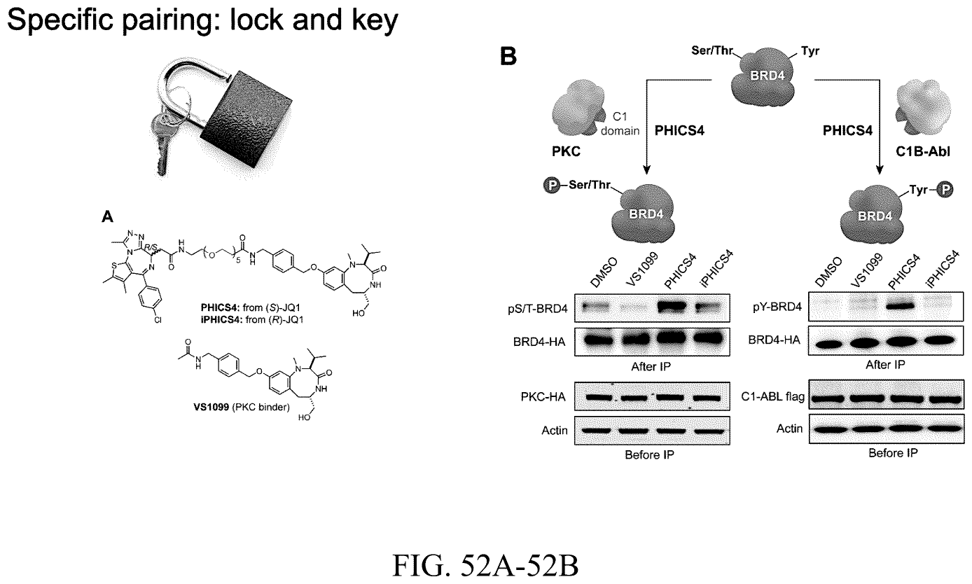

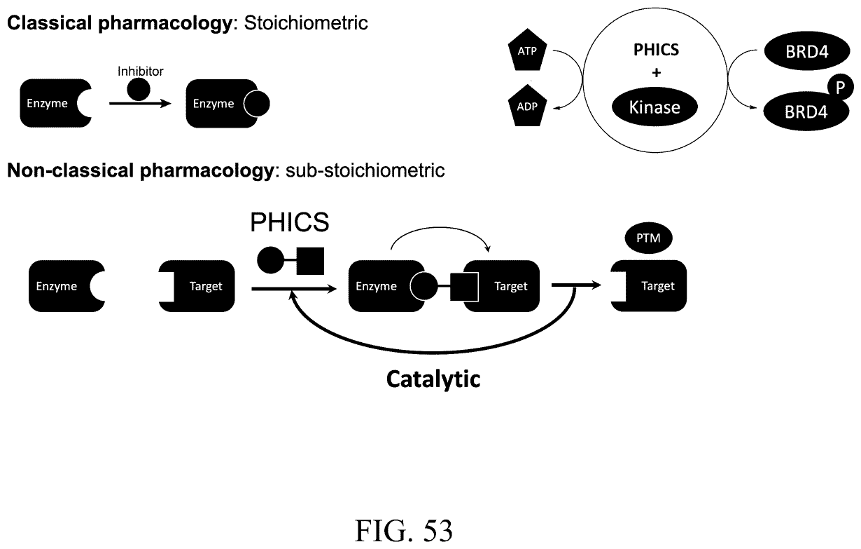

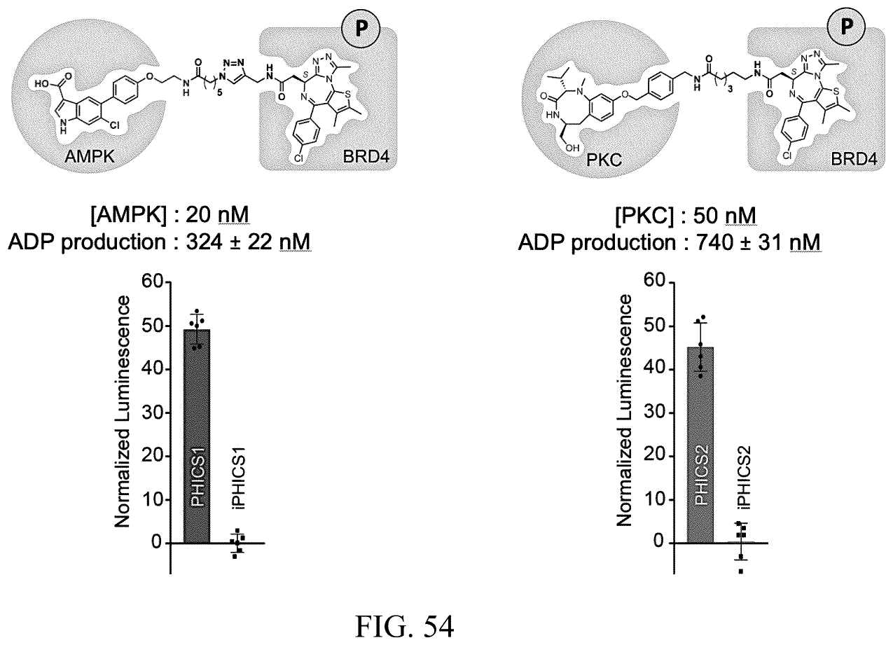

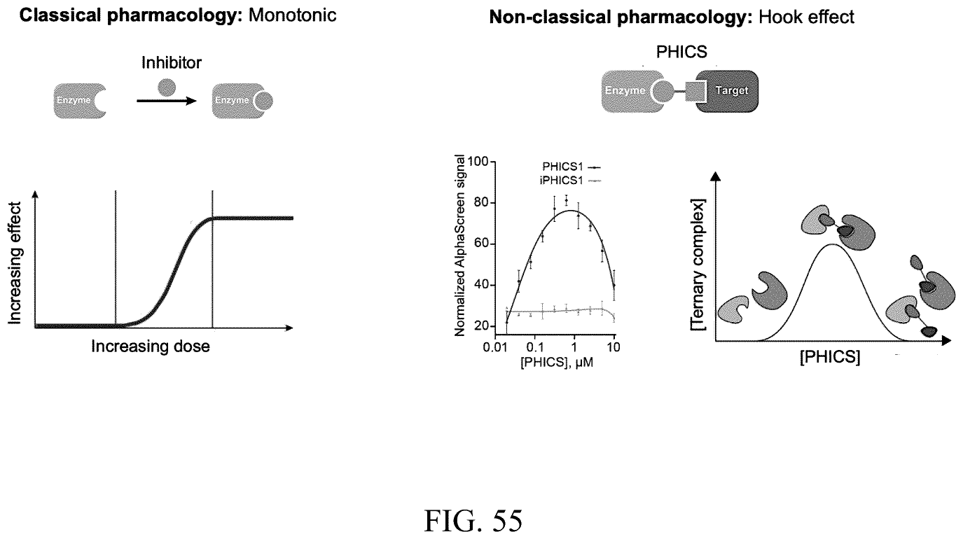

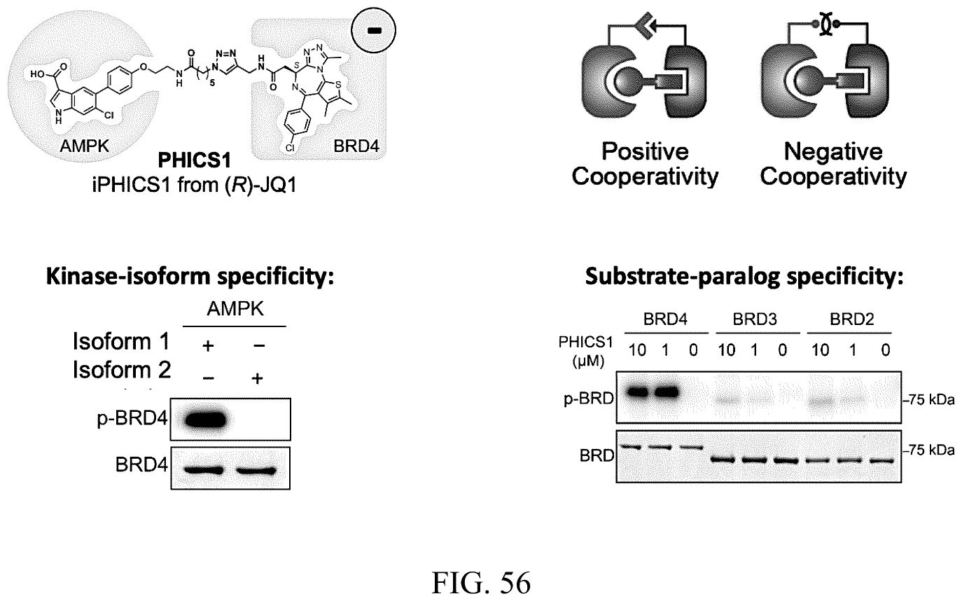

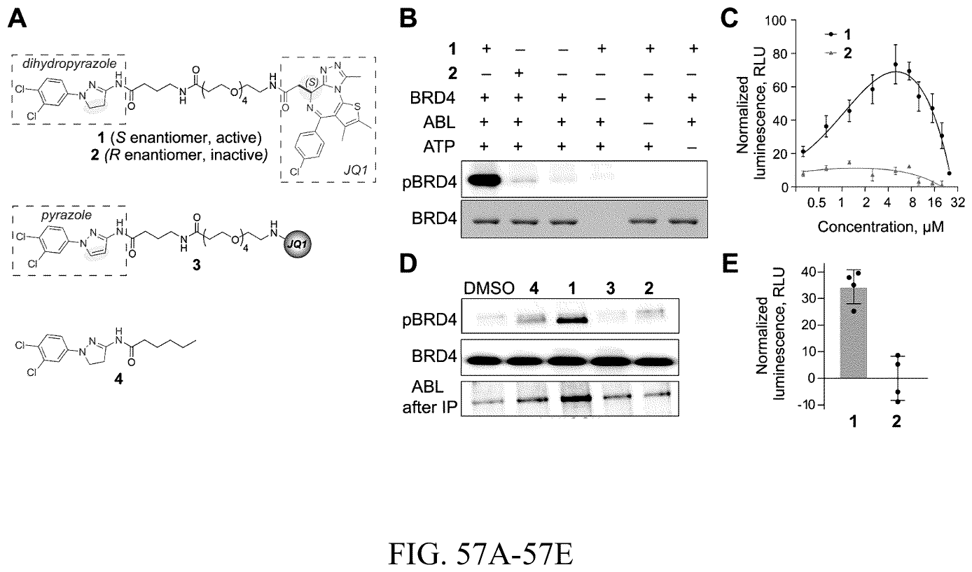

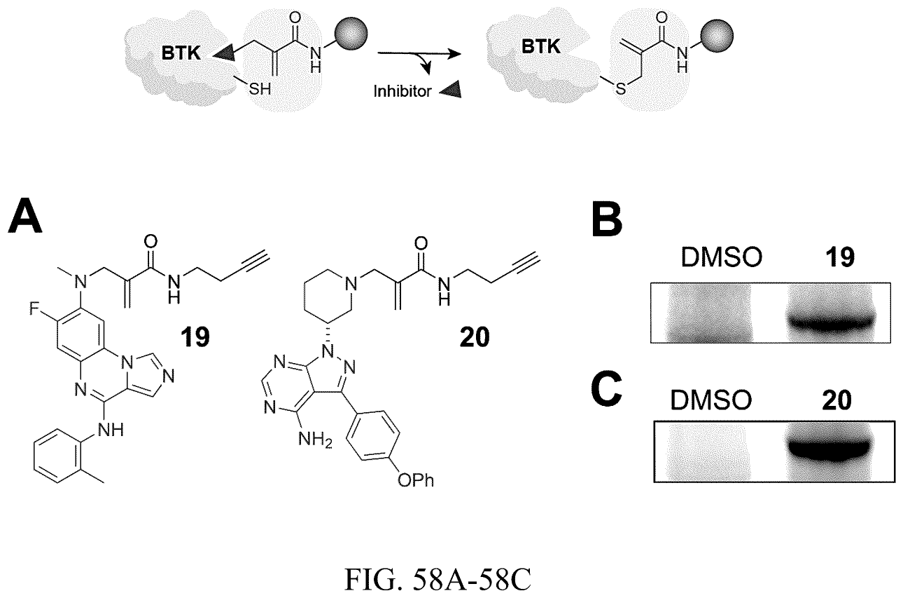

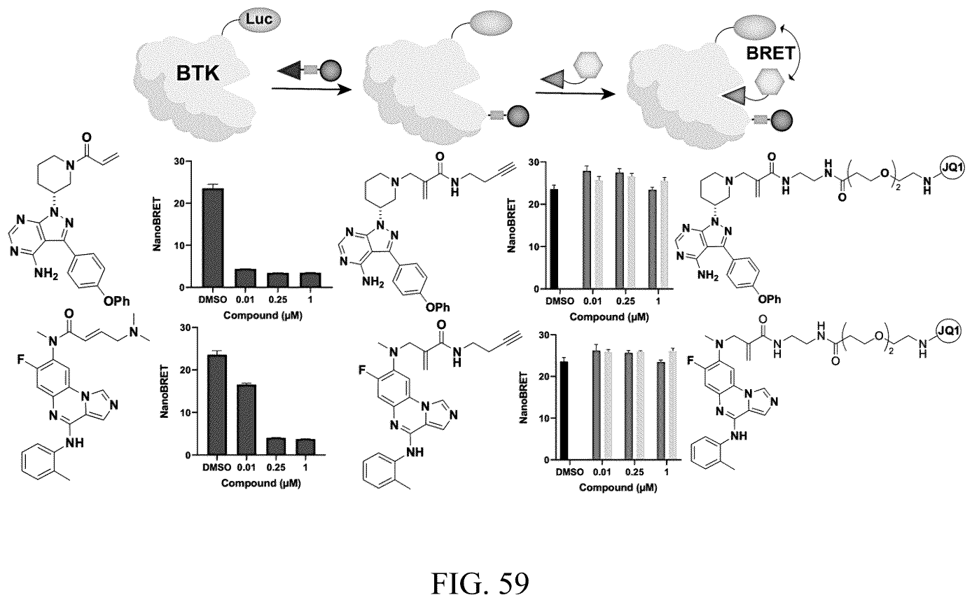

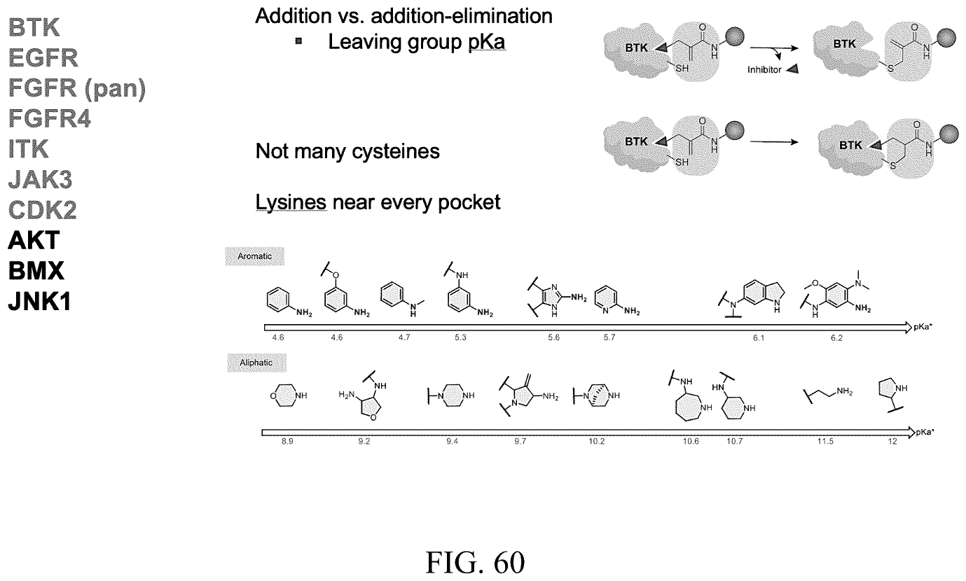

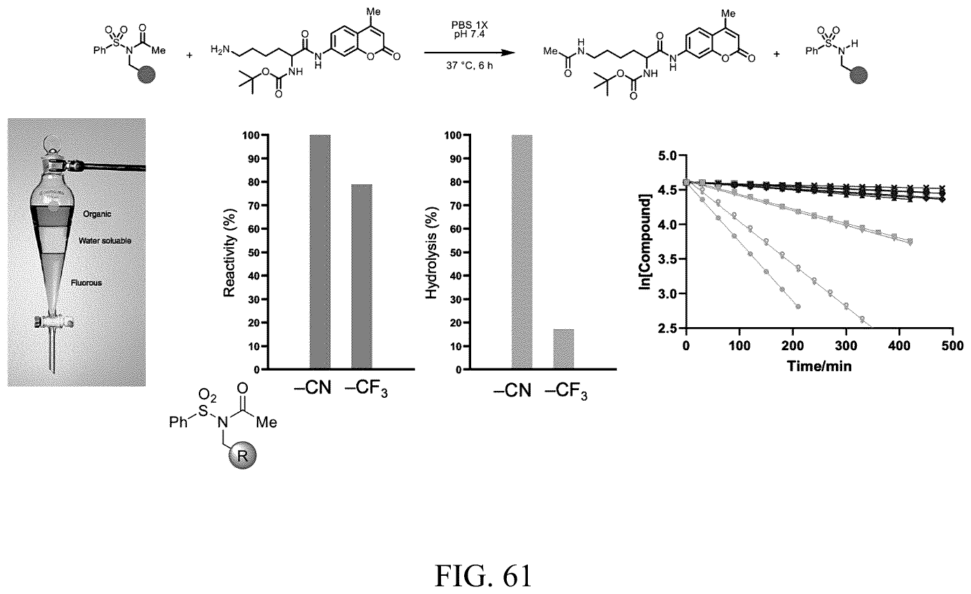

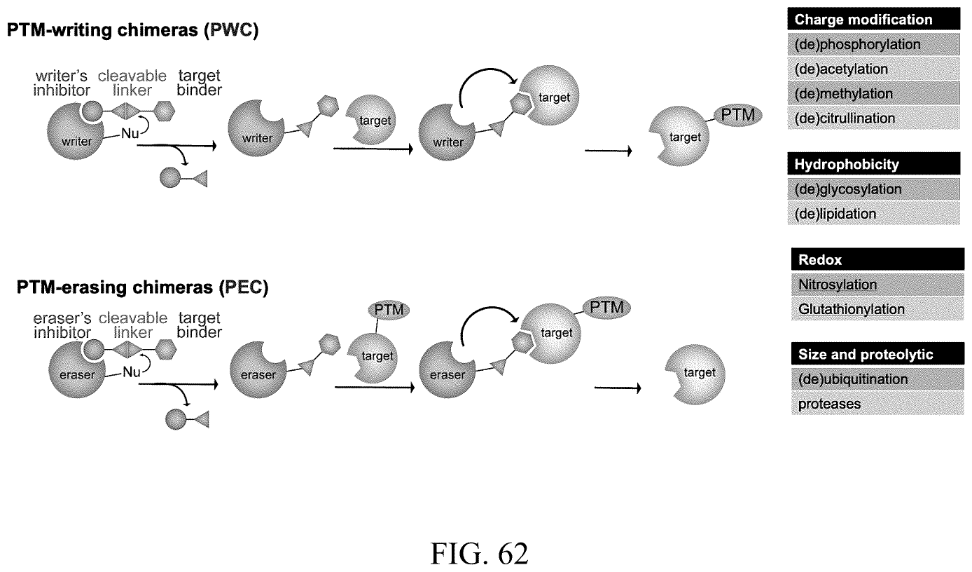

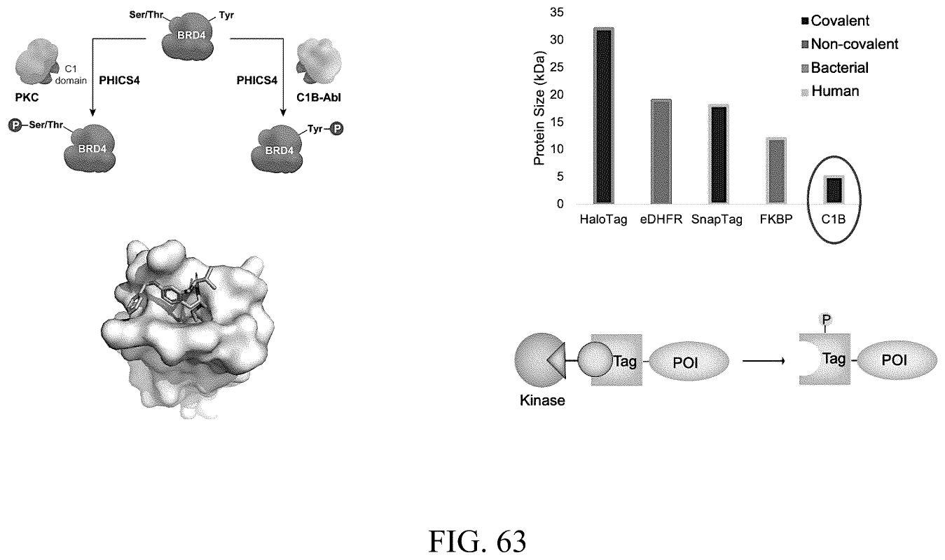

In one example embodiment, the target binding moiety is an oligosaccharide binding moiety. Oligosaccharide binding moieties include small molecules. For example, small molecules that include boronic acid are typically used to bind to oligosaccharides. See e.g. Jin S., et al. “Carbohydrate Recognition by Boronolectins, Small Molecules, and Lectins. Med. Res. Rev. 2009, herein incorporated by reference. Other oligosaccharide binding moieties include carbohydrate binding proteins, are important targets when considering antiviral and anticancer drugs. The localizing moiety can be, for example, a lectin, facilitating interaction sites for carbohydrates. Exemplary molecules include small molecule boronolectins, nucleic acid-based boronolectins, and peptidoboronolectins. See, e.g. Jin et al., Med. Res Rev. 2010 March; 30(2): 171-257; doi: 10.1002/med.20155, incorporated herein by reference, specifically FIGS. 1-50 for binding molecules and the complexes formed. Publicly available computational methods are available using developed bioinformatics to select small molecules capable of binding carbohydrates, see, e.g., Zhao et al., Current Protocols in Protein Science 94:1 10.1002/cpps.75; Shionyu-Mitsuyama C, Shirai T, Ishida H, Yamane T (2003) Protein Eng 16:467-478; and Kulharia M, Bridgett S J, Goody R S, Jackson R M (2009) InCa-SiteFinder: a method for structure-based prediction of inositol and carbohydrate binding sites on proteins. J Mol Graph Model 28:297-303.

Analysis of binding site residues along with stabilizing residues in protein-carbohydrate complexes can allow for identification of folding and binding of the complexes to understand interactions in addition to non-covalent interactions of hydrogen bonding and non-polar interactions. See, e.g., Shanmugam et al., doi.: 10.2174/0929866525666180221122529. Utilizing publicly available tools, carbohydrate binding moieties, including binding sites and predicted folding can be used for the design of chimeric small molecules comprising such a carbohydrate binding moiety.

### Lipid Binding Moieties

In one example embodiment, the target binding moiety is a lipid binding moiety. Lipid binding moieties can be utilized as target binding moieties in the chimeric small molecules disclosed herein. As regulators of cellular stabilization and signaling, modifications in their composition, distribution or trafficking would be useful in treatment, regulation and/or modification of pathways, processes and conditions. Lipids include charged lipids, e.g. phosphatidylserine (PS), phosphatidic acid (PA), phosphatidylinositol (PI), and the PI-phosphate, -bisphosphate, and -trisphosphate (PIPs—a family of seven anionic charged lipids), and ganglioside (GM). Zwitterionic lipids, e.g., phosphatidylcholine (PC), phosphatidylethanolamine (PE), and sphingomyelin (SM) lipids, Ceramides (CER), diacylglycerol (DAG), and lysophosphatidylcholine (LPC) lipids, sphingolipids, glycerophospholipids, cholesterol, phosphatidylglycerols.

Lipid binding moieties may be incorporated into chimeric small molecules. For example, certain steroids are capable of targeting and binding to lipids. Other lipid binding moieties such as proteins can either bind lipids specifically, where a clear binding site for a given lipid can be identified, or nonspecifically, where lipids act as a medium, and physical properties like thickness, fluidity, or curvature regulate the protein function. Phosphoinositide binding domains such as FYVE or PX, or the FRRG motif in the B-propeller of PROPPINs are more common domains that can be used to identify lipid binding proteins. The FYVE domain, named after the first four proteins to contain the motif (Fab1, YOTB, Vac1 EEA1) contains several conserved regions, which can also be utilized to identify related domains. See, e.g., A. H. Lystad, A. Simonsen Phosphoinositide-binding proteins in autophagy, FEBS Lett., 590 (2016), pp. 2454-2468, 10.1002/1873-3468.12286. Additional FYVE domain-containing proteins include SARA, FRABIN, DFCP1 FGD1, ANKFY1, EEA1 FGD1, FGD2, FGD3, FGD4, FGD5, FGD6, FYCO1, HGS MTMR3, MTMR4, PIKFYVE, PLEKHF1, PLEKHF2, RUFY1, RUFY2, WDF3, WDFY1, WDFY2, WDFY3, ZFYVE1, ZFYVE16, ZFYVE19, ZFYVE20, ZFYVE21, ZFYVE26, ZFYVE27, ZFYVE28, ZFYVE9.

Eukaryotic cells can degrade intracellular components through a lysosomal degradation pathway called macroautophagy, with pathway malfunction linked to several diseases. Dikic et al., Mechanism and medical implications of mammalian autophagy. Nat. Rev. Mol. Cell Biol., 19 (2018), pp. 349-364, doi: 10.1038/s41580-018-0003-4. Accordingly, autophagy related (ATG) proteins may be utilized as lipid binding moiety in the present invention, including LC3A, LC3B, LC3C, GABARAP, GABARAPL1 and GABARAPL2. De la Ballina (2019), doi.org/10.1016/j.jmb.2019.05.051. Lipid-binding proteins include protein HCLS1 binding protein 3 (HS1BP3) that is able to negatively regulate the activity of phospholipase D1 (PLD1).

### Target Protein Binding Moiety

As detailed herein, target protein binding moieties for exemplary proteins of interest to be targeted for modification are provided. The target protein binding moiety is chosen based on the desired association and modification. Accordingly, the modifications desired, which may be tailored based on a particular condition, disease, treatment, or other desired effect, will be a design consideration when choosing the protein binding moiety.

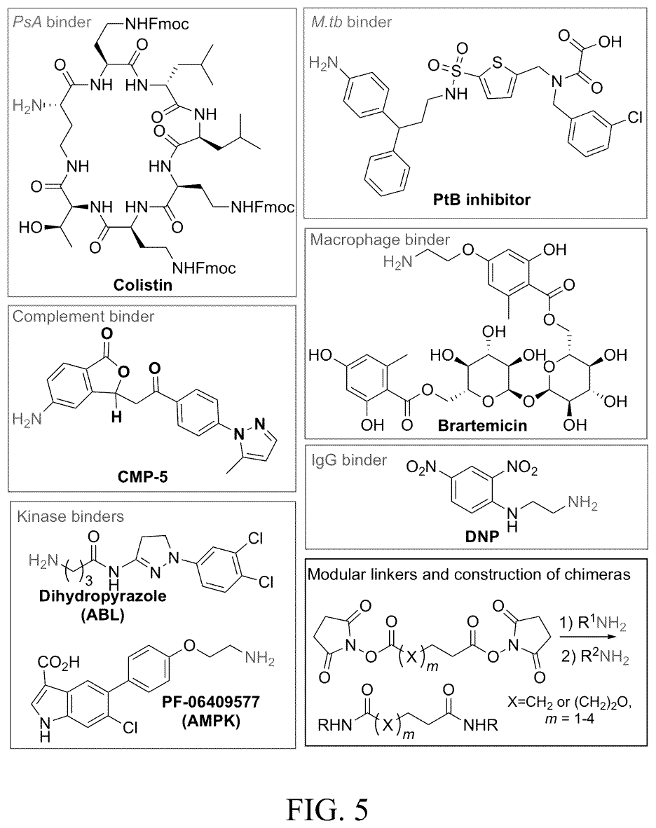

The target protein binding moiety may be chosen for a specific protein of interest, which may be located in different localization sites of the cell, e.g. nucleus, cytoplasm, mitochondria, cell surface. Example target protein binding moieties are disclosed for example in, Sun et al., Signal Transduction and Targeted Therapy, 4:64 (2019), which provides exemplary proteins and corresponding ligands (i.e. target polypeptide binding moieties, see in particular FIGS. 5-48, which is incorporated herein by reference). The target protein binding moiety may bind to proteins which undergo conformation change upon binding.

The target protein binding moiety may bind to proteins which undergo conformation change upon binding, for example, an androgen receptor (AR). In one embodiment, activation of the protein results in modification of the target substrate by the protein at one or more new modification sites that would otherwise remain unmodified by the protein when not activated by the chimeric small molecule. The target substrate is not required to be a natural substrate of the protein. The target substrate may be a protein, and discussion herein of genes includes the products of the gene expression.

In one embodiment, the target protein binding moiety is capable of binding a protein that is an ATPase or GTPase. Exemplary GTPases may be from the Ras, Rho, Rab, Arf or Ran family, see, e.g. Yoshimi Takai, Takuya Sasaki, and Takashi Matozaki, Small GTP-Binding Proteins, Physiological Reviews 2001 81:1, 153-208; doi: 10.1152/ohysrev.2001.81.1.153. Exemplary molecules targeting may include molecules such as Ibrutinib (BTK), Dsatinib (BCR-ABL), MRTX (KRAS), MI-1061 (MDM2), Gelfitinib (EGFR), Palbociclib (CDK4/6) and Foretinib (C-MET), or analogs thereof.

In one example embodiment the target substrate is modified with an orthogonal tag, e.g. FKBP12^{F36V} SNAP-, CLIP-, ACP- and MCP-tags, and the target binding moiety is a binder of the orthogonal tag. See, e.g. neb.com/tools-and-resources/feature-articles/snap-tag-technologies-novel-tools-to-study-protein-function, incorporated herein by reference.

The following provide examples of further, non-limiting, target protein binding moieties to various target proteins of interest in oncology and infectious disease contexts and the use of which will discussed further in the Methods of Use section below.

### KRAS

In one example embodiment, the target protein binding moiety is a KRAS binding moiety. In one example embodiment, the target protein binding moiety is a KRAS binder according selected from the group consisting of:

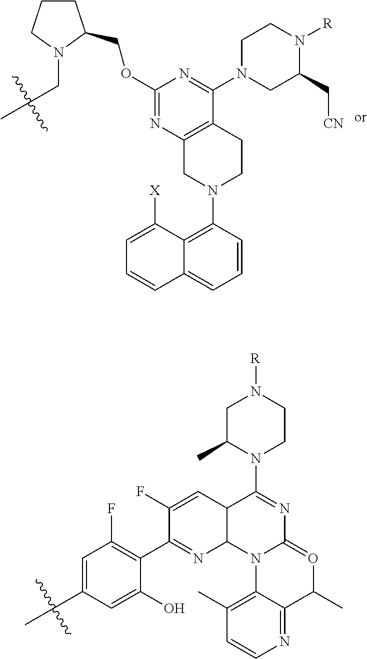

wherein R is a covalent warhead; X is the formula

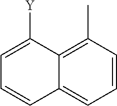

and Y is selected from the group consisting of: H, alkane, alkene, alkyne, amine, nitrile, nitro, ether, alcohol, thiol, sulfone, sulfonate, halogen, carbonyl, acyl, ketone, carboxylate ester, amide, enone, anhydride, imide, cyclic hydrocarbon, an unsaturated cyclic hydrocarbon, a heterocycle, one or more fused rings thereof, or an aliphatic halides such as —OCF₂Cl.

In one example embodiment, the target binding moiety is a hydrogen bond surrogate (HBS) Son of Sevenless (SOS) peptide mimics (PM). In one example embodiment, the HBS-SOS-PM is HBS 1-7 according to the sequences: XFE*GIYRTDILRTEEGN-NH2 (SEQ ID NO: 4); XFE*GIYRTELLKAEEAN-NH2 (SEQ ID NO: 5); XFE*GIYRLELLKAEEAN-NH2 (SEQ ID NO: 6); XFE*GIYRLELLK-NH2 (SEQ ID NO: 7); XFE*AIYRLELLKAEEAN-NH2 (SEQ ID NO: 8); XFE*GIYRLELLKAibEEAibN-NH2 (SEQ ID NO: 9); and XAE*GIYRLELLKAEAAA-NH2 (SEQ ID NO: 10), respectively, wherein X denotes a 4-pentenoic acid residue and the asterisk (*) denotes N-allyl residue (*G, N-allylglycine). In one example embodiment, the target binding moiety is a KRAS binding molecule HB3 according to the formula: XFE*GIYRLELLKAEEAN-NH2 (SEQ ID NO: 11). In one example embodiment, the target binding moiety is a KRAS binding molecule HB7 according to the formula: XAE*GIYRLELLKAEAAA-NH2 (SEQ ID NO: 12). See Nickerson et al., An Orthosteric Inhibitor of the RAS-SOS Interaction, doi: 10.1016/B978-0-12-420146-0.00002-0 incorporated herein by reference in its entirety with specific mention of Table 2.1.

In one example embodiment, the target binding moiety is a KRAS binding molecule according to the formula:

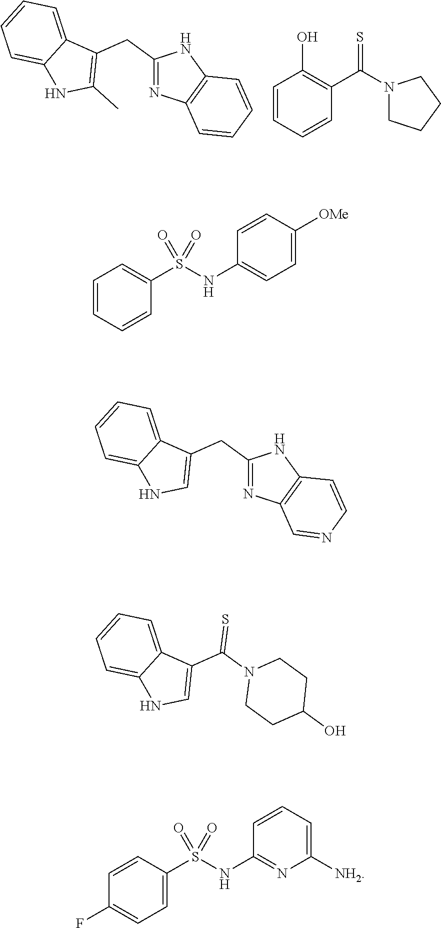

In one example embodiment, the target binding moiety is a KRAS binding moiety according to the formula:

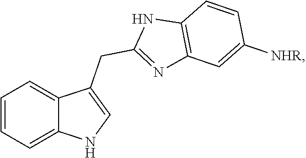

wherein R may be H, Gly, Ala, β-Ala, Val, Ile, Pro, or any other feasible substituent known in the art. In one example embodiment, the target binding moiety is a KRAS binding moiety is an indole, phenol, sulfonamide, or any modified version thereof. See Sun et al., Angew Chem Int Ed Engl. 2012 Jun. 18; 51(25): 6140-6143. doi: 10.1002/anie.201201358, herein incorporated by reference in its entirety.

In one example embodiment, the target binding moiety is a KRAS binding molecule according to the formula:

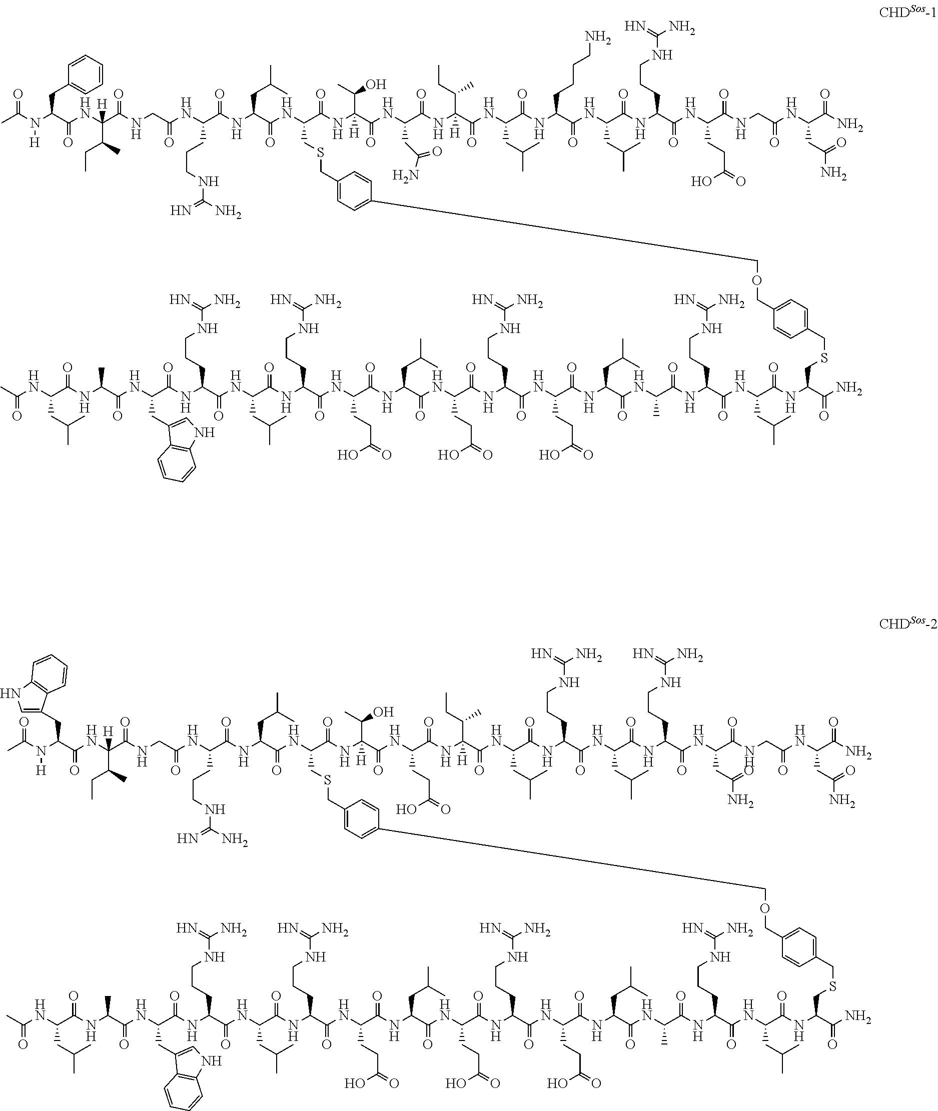

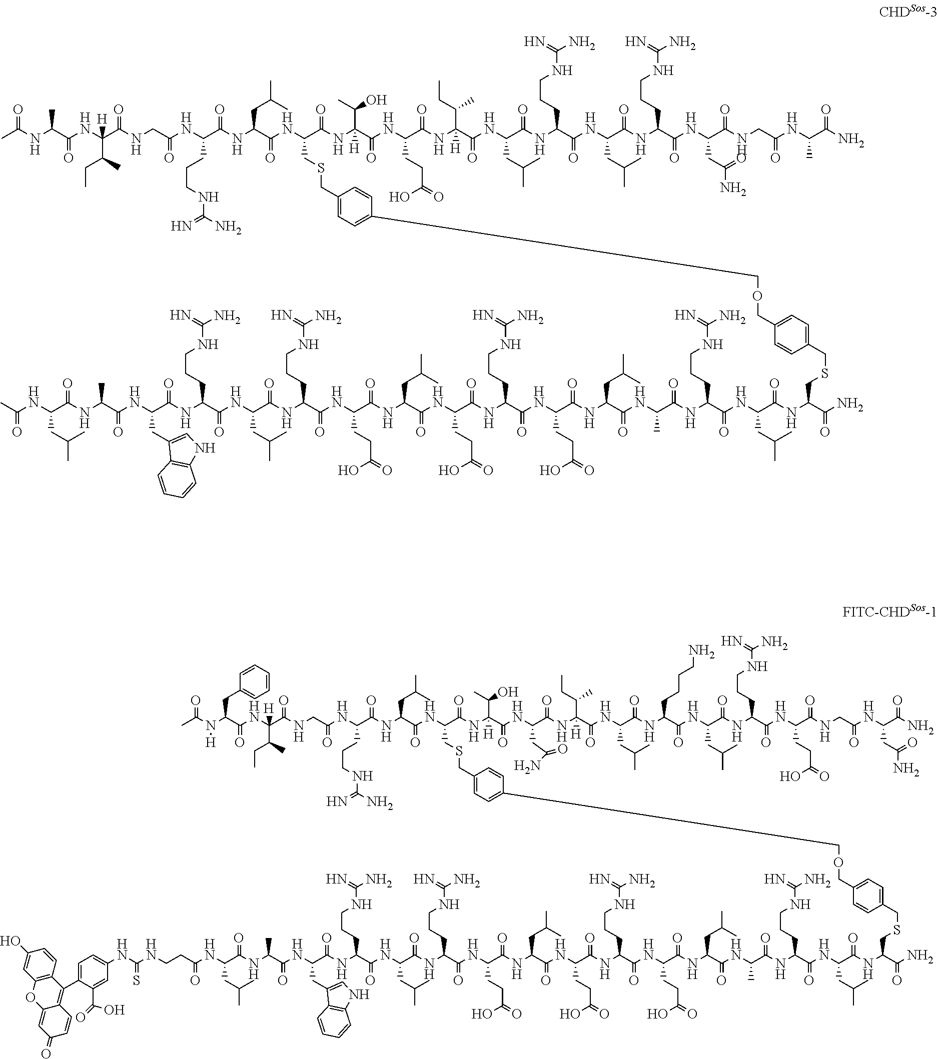

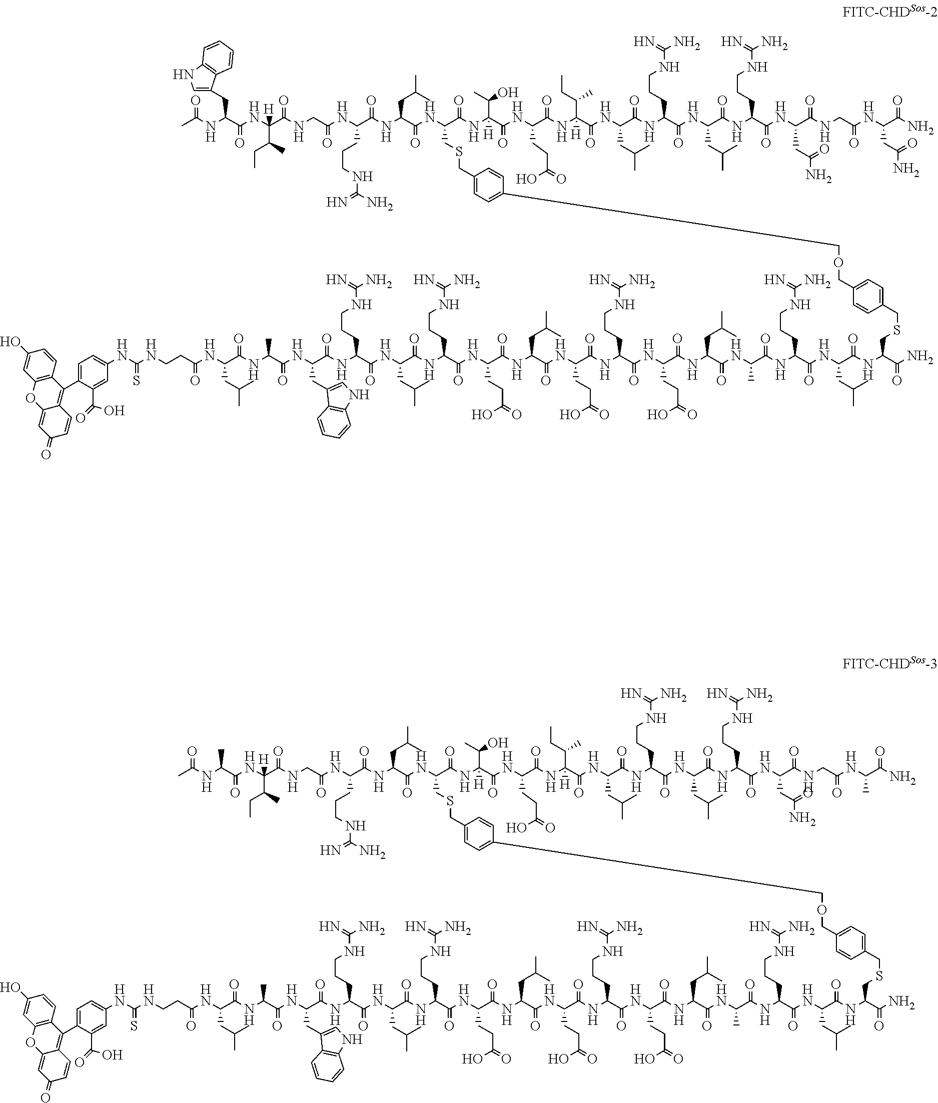

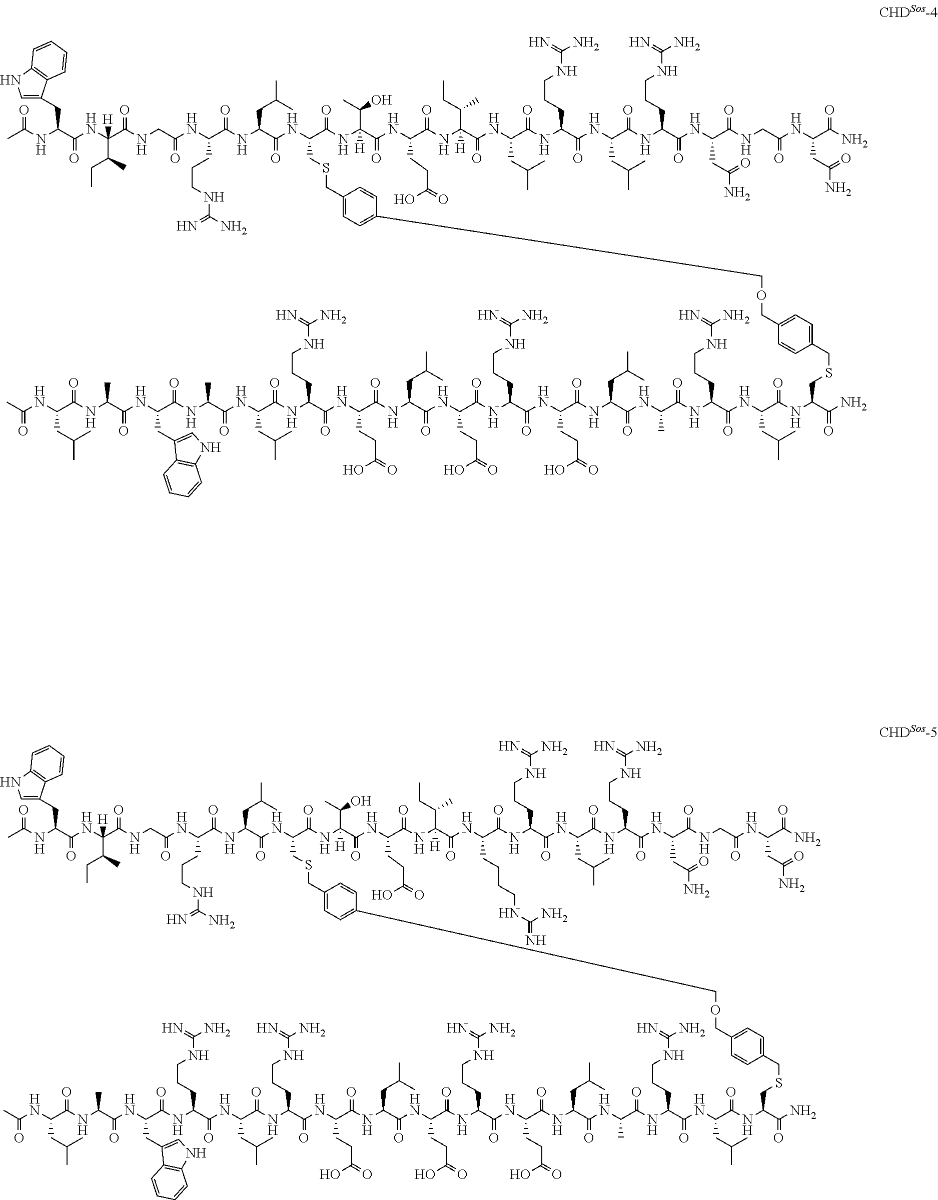

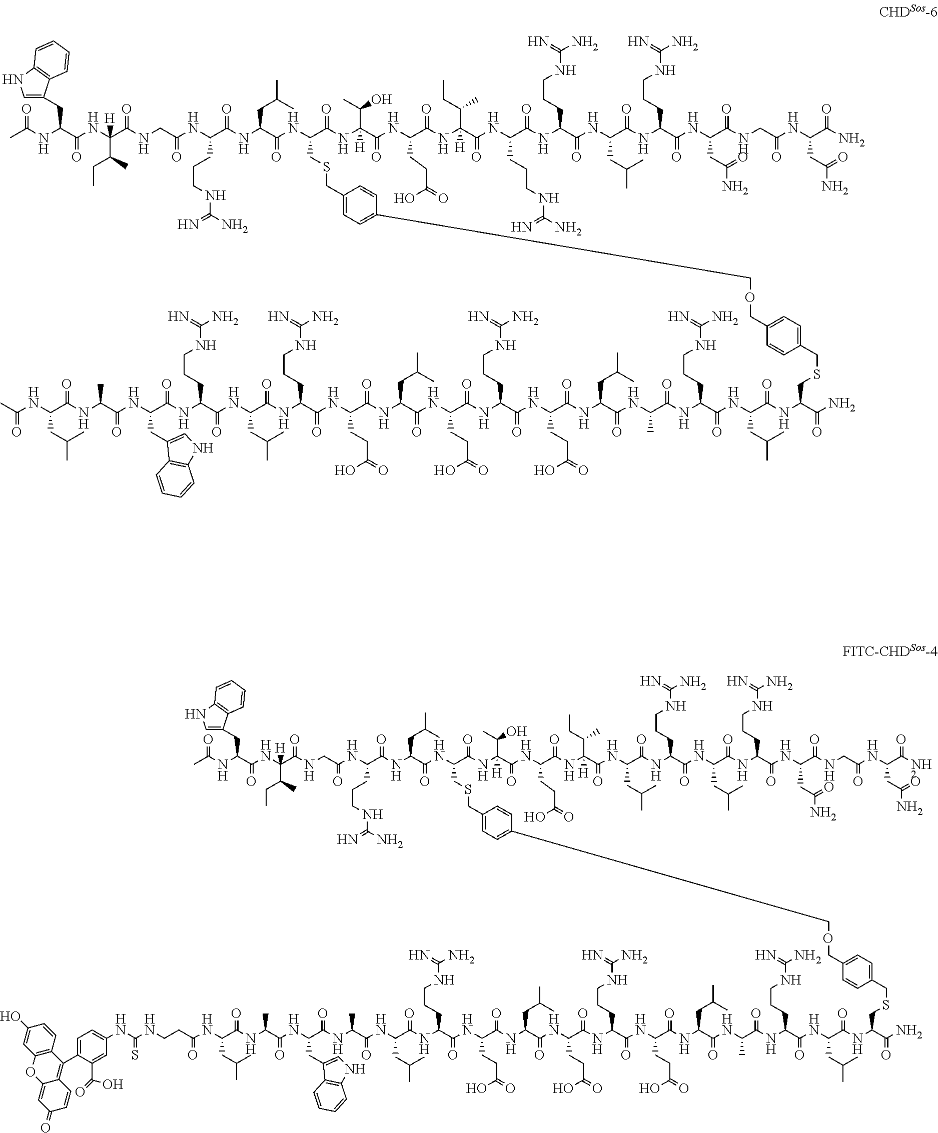

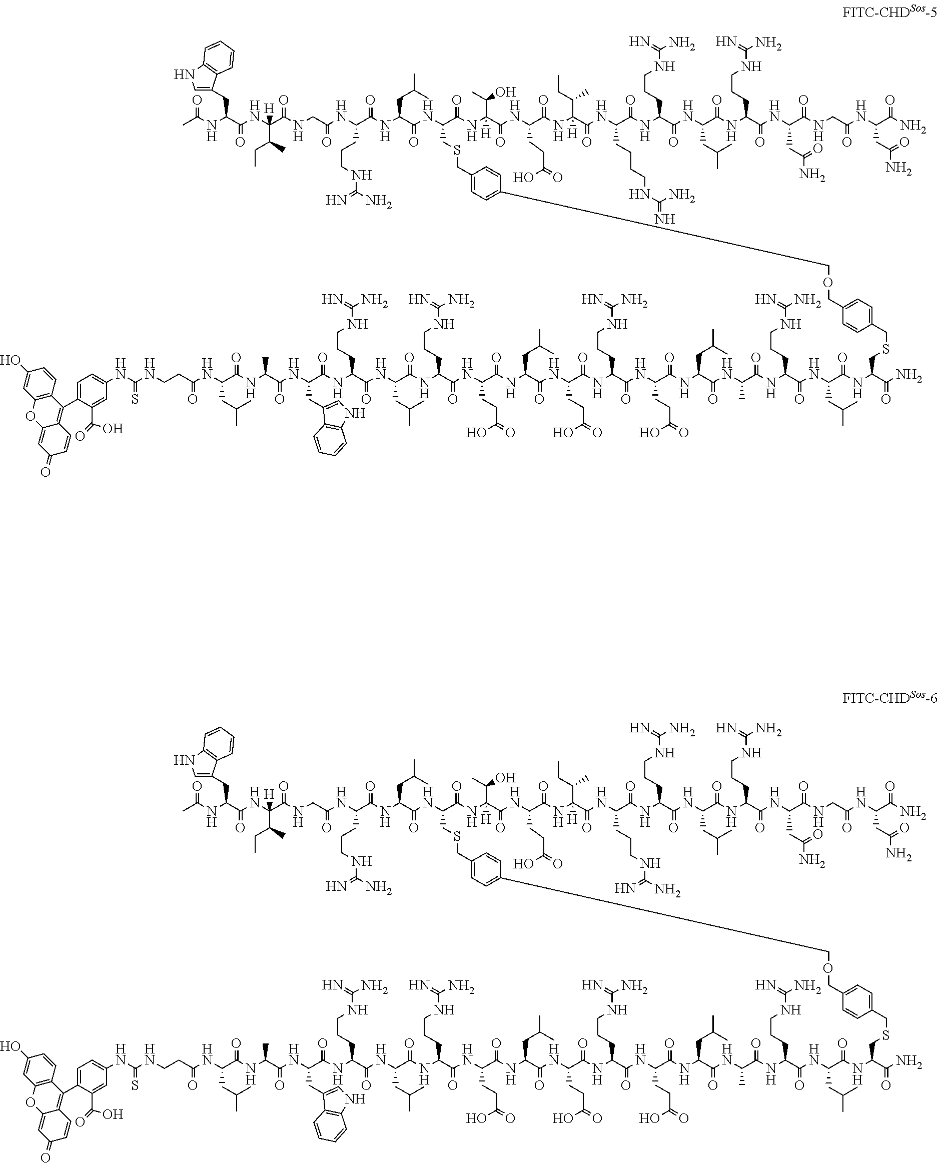

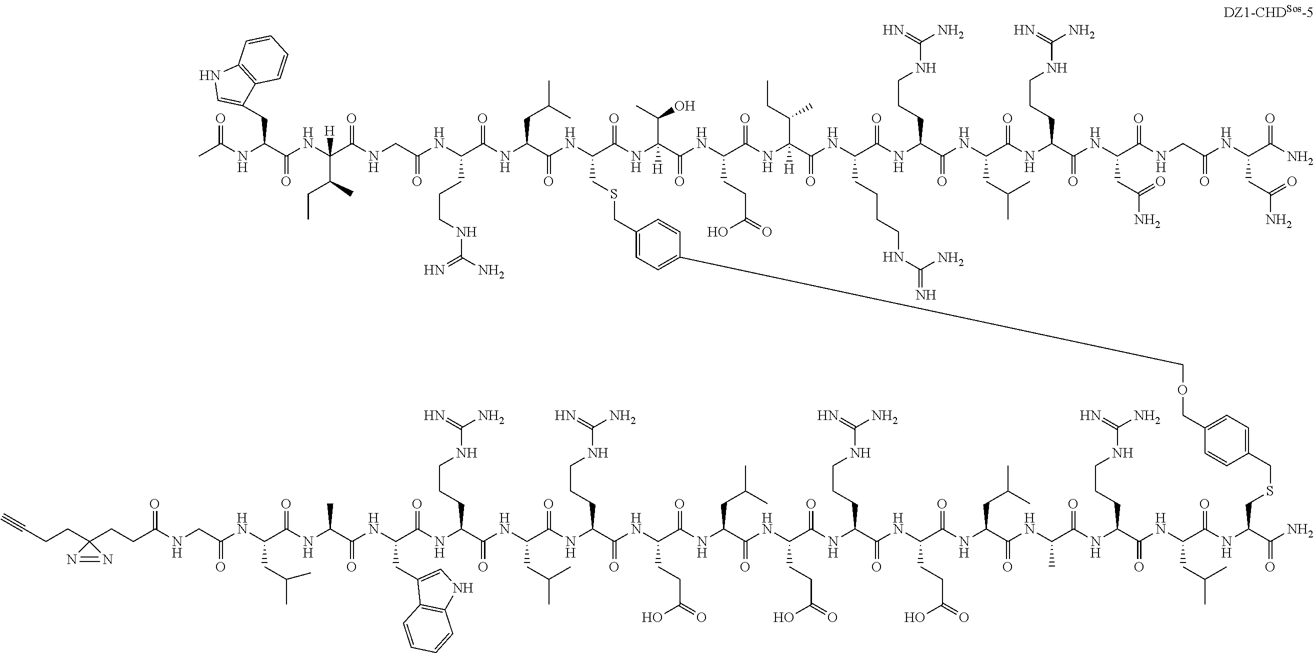

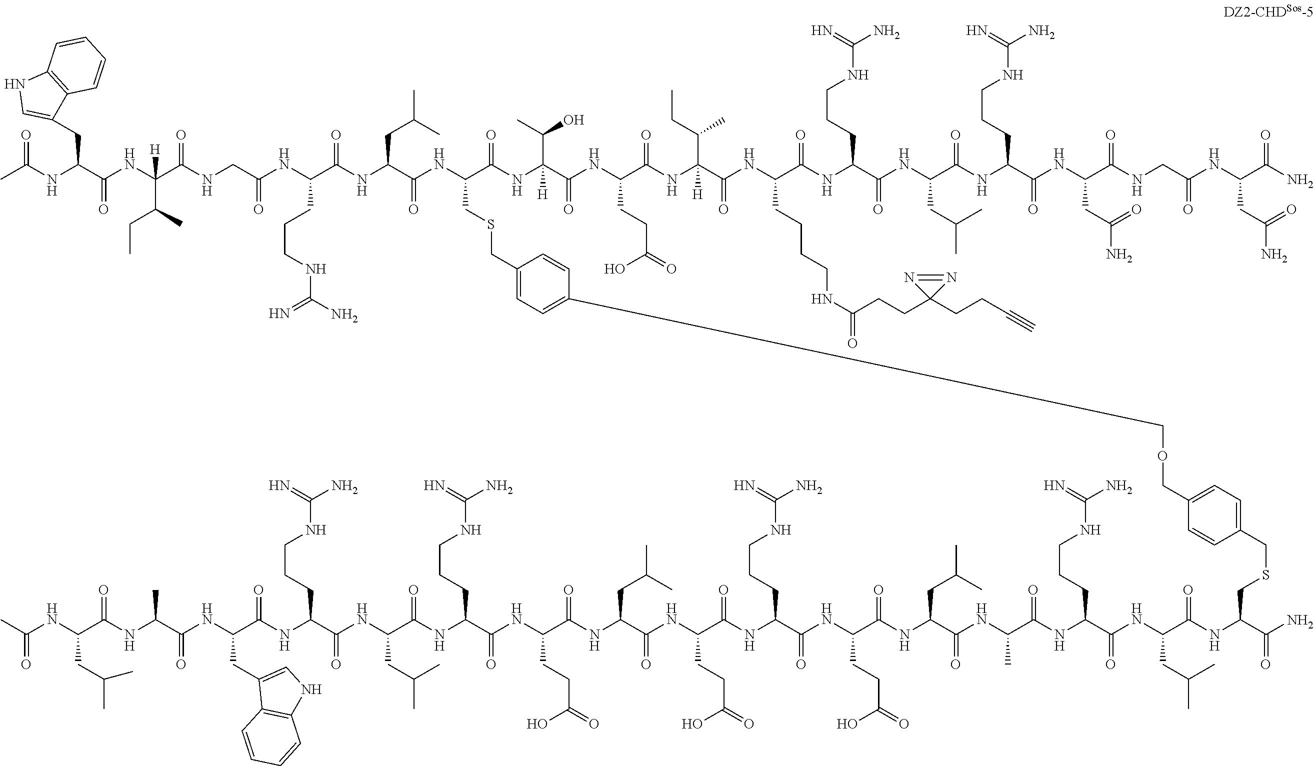

In one example embodiment, the target binding moiety is a SOS peptide mimic according to the formula: Ac-FIGRLCTEILKLREGN-NH2 (SEQ ID NO: 13; Ac-LAWRLRELERELARLC-NH2 (SEQ ID NO: 14); Ac-WIGRLCTEILRLRNGN-NH2 (SEQ ID NO: 15); Ac-LAWRLRELERELARLC-NH2 (SEQ ID NO: 16); Ac-AIGRLCTEILRLRNGA-NH2 (SEQ ID NO: 17); Ac-LAWRLRELERELARLC-NH2 (SEQ ID NO: 18); Ac-WIGRLCTEILRLRNGN-NH2 (SEQ ID NO: 19); Ac-LAWALRELERELARLC-NH2 (SEQ ID NO: 20); Ac-WIGRLCTEIR^{H}RLRNGN-NH2 (SEQ ID NO: 21); Ac-LAWRLRELERELARLC-NH2 (SEQ ID NO: 22); Ac-WIGRLCTEIRRLRNGN-NH2 (SEQ ID NO: 23); Ac-LAWRLRELERELARLC-NH2 (SEQ ID NO: 24); Ac-WIGRLCTEILRLRNGN-NH2 (SEQ ID NO: 25); Ac-LAWRLRELERELARLC-NH2 (SEQ ID NO: 26); Ac-FIGRLCTEILKLREGN-NH2 (SEQ ID NO: 27); FITC-AβLAWRLRELERELARLC-NH2 (SEQ ID NO: 28); Ac-WIGRLCTEILRLRNGN-NH2 (SEQ ID NO: 29); FITC-AβLAWRLRELERELARLC-NH2 (SEQ ID NO: 30); Ac-AIGRLCTEILRLRNGA-NH2 (SEQ ID NO: 31); FITC-AβLAWRLRELERELARLC-NH2 (SEQ ID NO: 32); Ac-WIGRLCTEILRLRNGN-NH2 (SEQ ID NO: 33); FITC-AβLAWALRELERELARLC-NH2 (SEQ ID NO: 34); Ac-WIGRLCTEIR^{H}RLRNGN-NH2 (SEQ ID NO: 35); FITC-AβLAWRLRELERELARLC-NH2 (SEQ ID NO: 36); Ac-WIGRLCTEIRRLRNGN-NH2 (SEQ ID NO: 37); FITC-AβLAWRLRELERELARLC-NH2 (SEQ ID NO: 38); Ac-WIGRLCTEIR^{H}RLRNGN-NH2 (SEQ ID NO: 39); DZ-GLAWRLRELERELARLC-NH2 (SEQ ID NO: 40); Ac-WIGRLCTEIK (DZ) RLRNGN-NH2 (SEQ ID NOS: 41-42); or Ac-LAWRLRELERELARLC-NH2 (SEQ ID NO: 43), wherein R^{H} is L-homoarginine; Aβ is L-β-alanine; DZ is diazirine photocrosslinker; and FITC is 5-fluorescein isothiocyanate linked via thiourea bond to N-terminal amine. See Hong et al., PNAS May 4, 2021 118(18)e2101027118; doi:10.1073/pnas.2101027118, herein incorporated by reference in its entirety with specific mention of Table S2.

In one example embodiment, the target binding moiety is a KRAS binding molecule according to the formula:

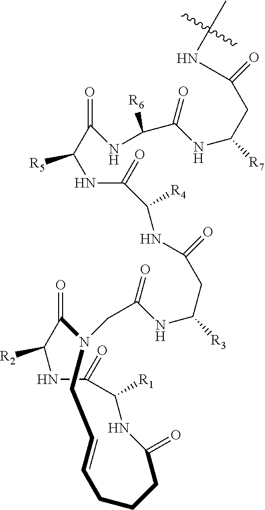

wherein the R groups may be any substituent known in the art. In one example embodiment, R4 is an electrophilic group. In one example embodiment the R4 is

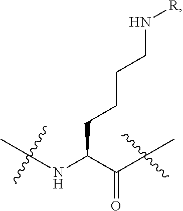

where R is H,

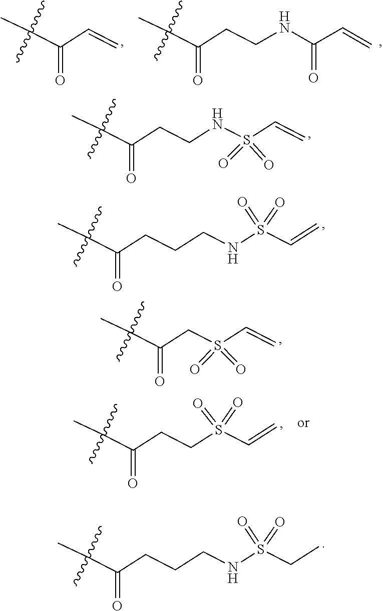

See Yoo et al., ACS Chem. Biol. 2020, 15, 6, 1604-1612, incorporated herein by reference in their entirety.

### FKBP12^{F36V}

In another example embodiment, the protein binding moiety can be designed to bind an FK506-binding protein (FKBP). The FKBP may be FKBP12, which binds to intracellular calcium release channels and TGF-β type I receptor. In one example embodiment, the FKBP protein binding moiety is an FKBP12^{F36V} binding molecule. In another example embodiment, the binding molecule is selected from

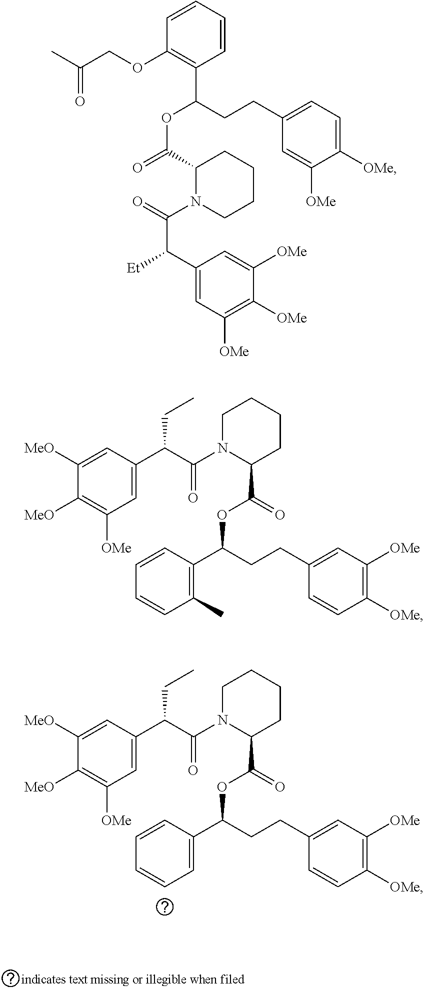

or an analog thereof.

Tyrosine phosphorylation on FGFR1 can trigger signaling cascade to induce PI3K/AKT/mTOR signaling and increased transcription of G-CSF, a blood growth factor. See, e.g. Turner et al, Nature Reviews Cancer 2010.

In one example embodiment, an ABL kinase is utilized to target the FKBP12^{F36V}. In an embodiment, the chimeric small molecule is selected from:

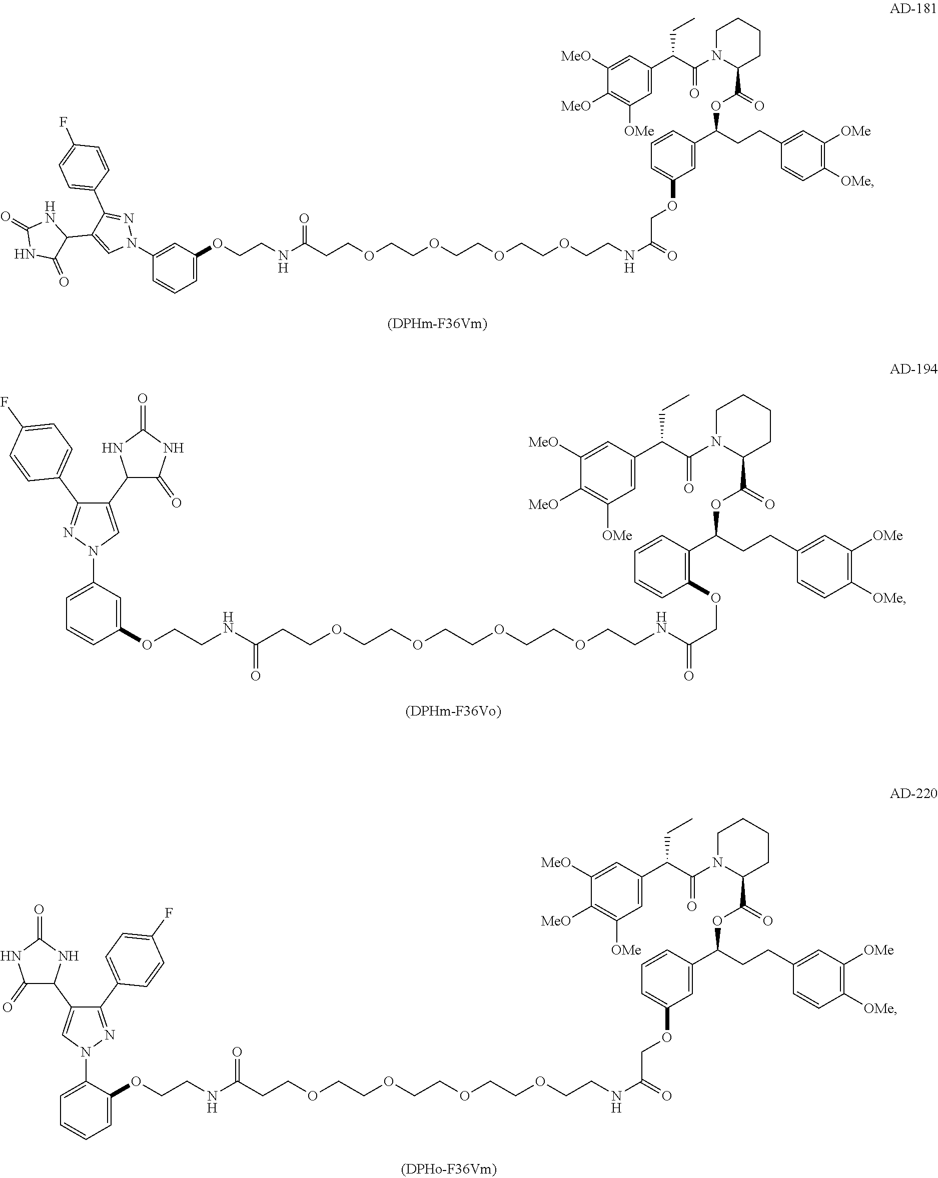

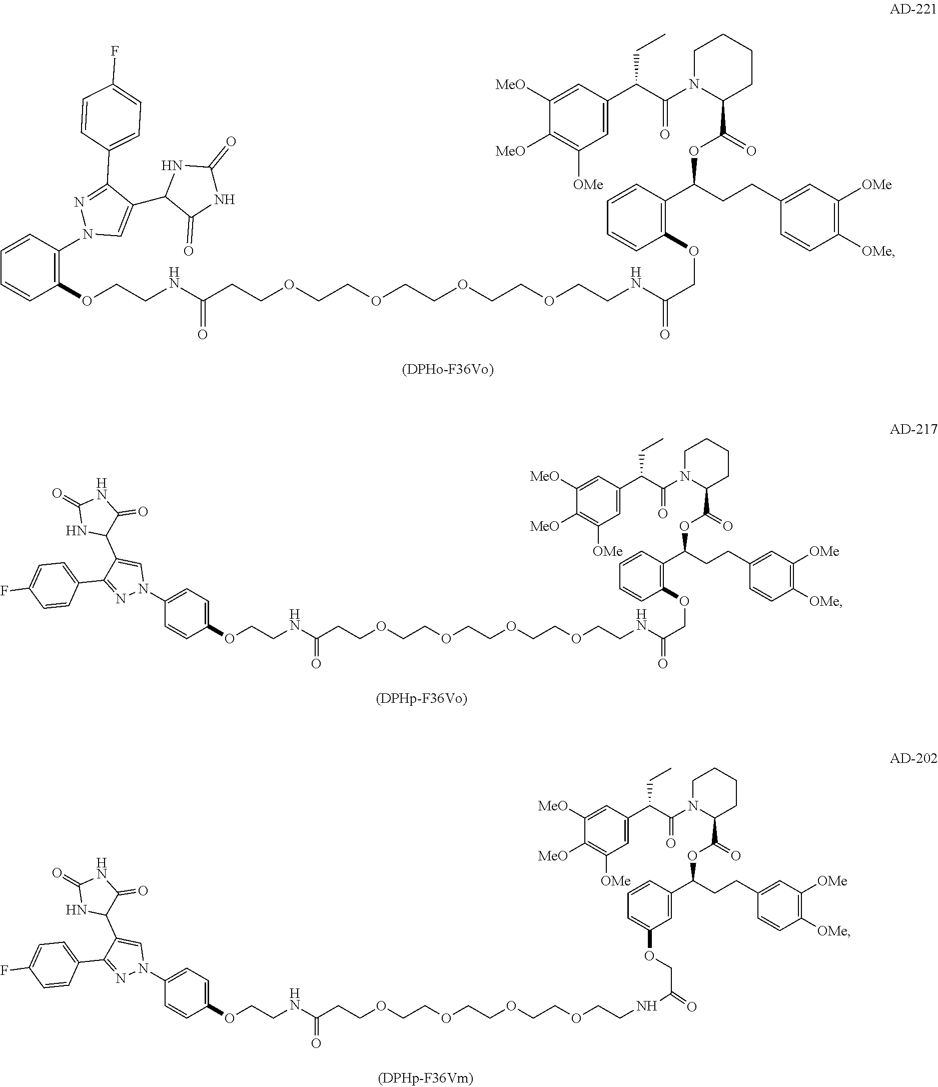

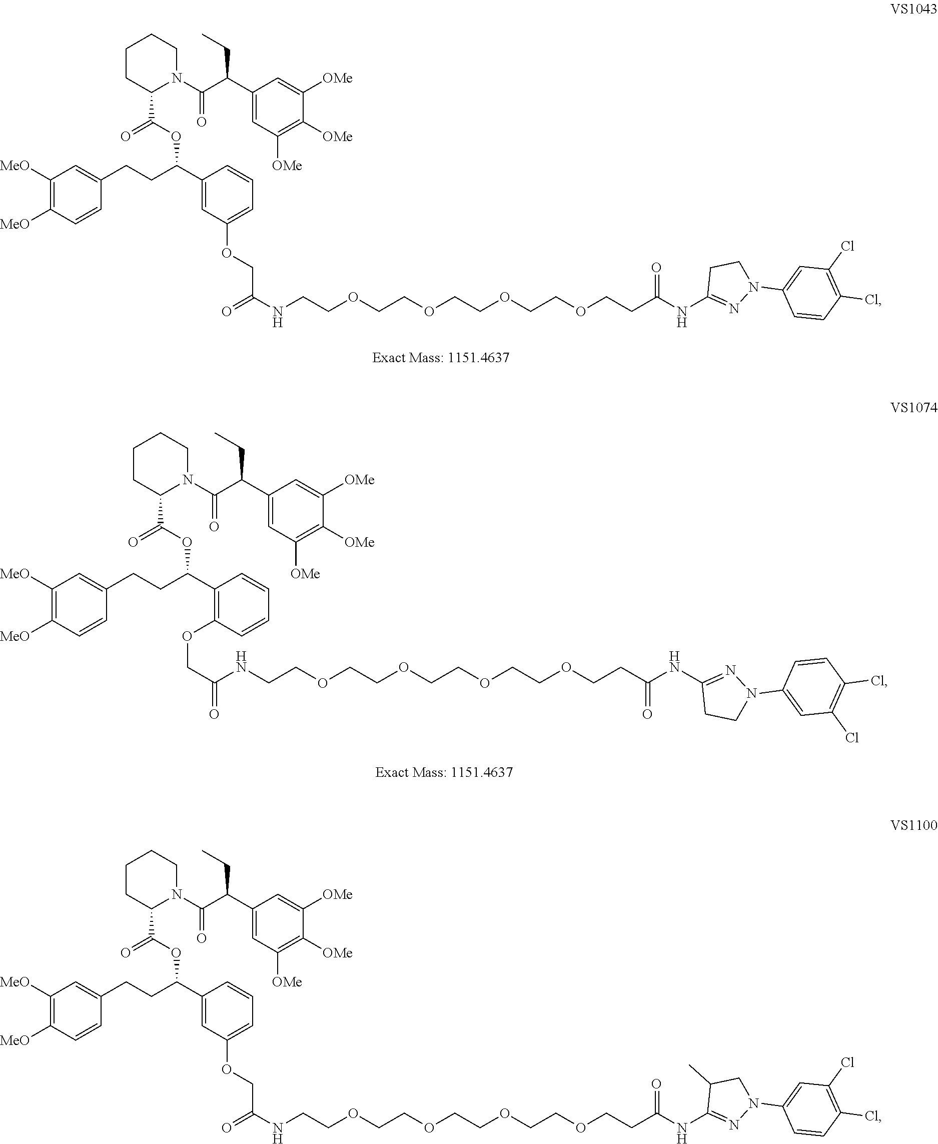 [SMILES: CC[C@H](C(=O)N1CCCC[C@H]1C(=O)O[C@@H](CCc1ccc(OC)c(OC)c1)c1cccc(OCC(=O)NCCOCCOCCOCCOCCC(=O)NC2=NN(c3ccc(Cl)c(Cl)c3)CC2)c1)c1cc(OC)c(OC)c(OC)c1 ; CC[C@H](C(=O)N1CCCC[C@H]1C(=O)O[C@@H](CCc1ccc(OC)c(OC)c1)c1ccccc1OCC(=O)NCCOCCOCCOCCOCCC(=O)NC1=NN(c2ccc(Cl)c(Cl)c2)CC1)c1cc(OC)c(OC)c(OC)c1 ; CC[C@H](C(=O)N1CCCC[C@H]1C(=O)O[C@@H](CCc1ccc(OC)c(OC)c1)c1cccc(OCC(=O)NCCOCCOCCOCCOCCC(=O)NC2=NN(c3ccc(Cl)c(Cl)c3)CC2C)c1)c1cc(OC)c(OC)c(OC)c1]

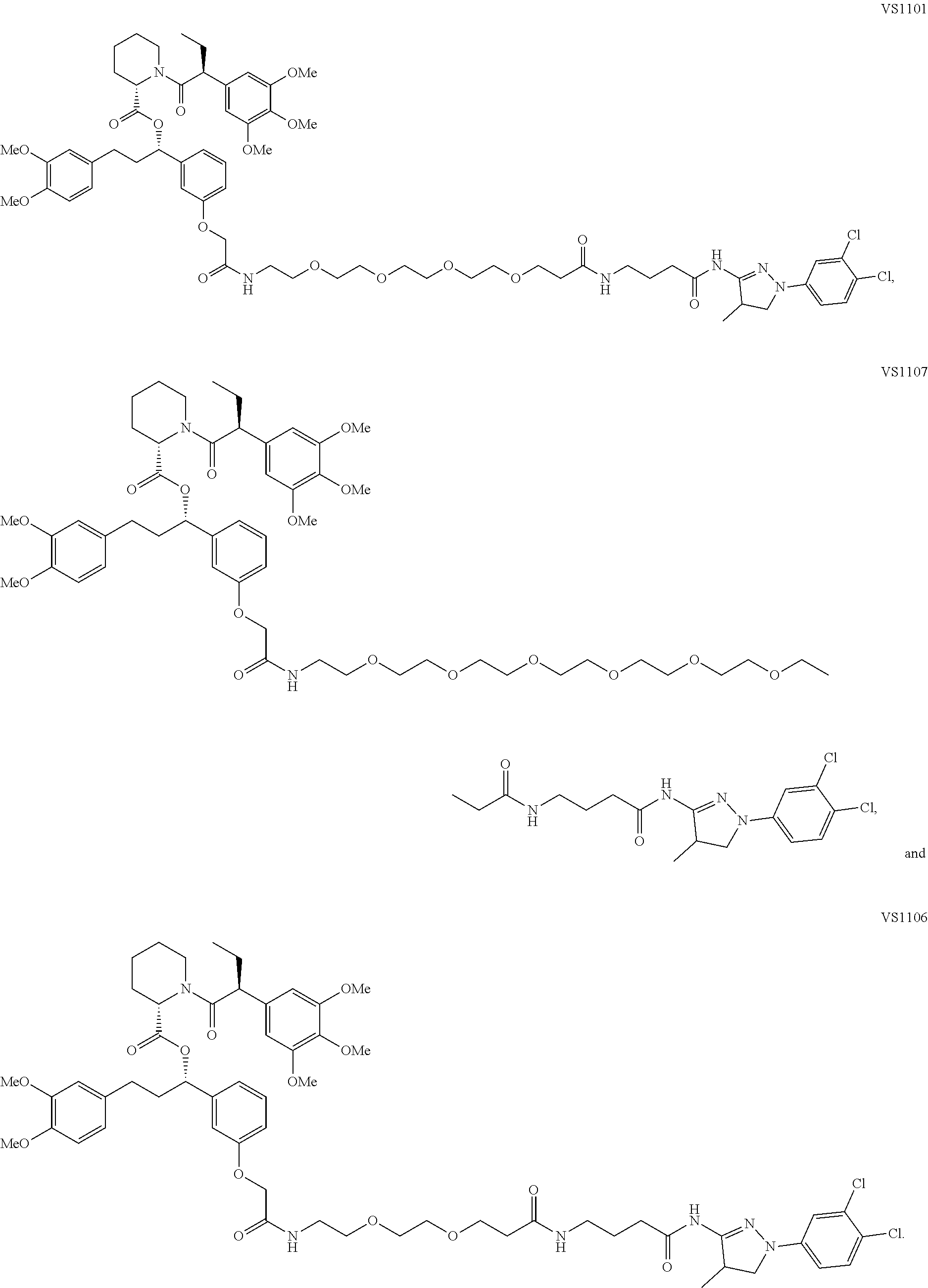

In one example embodiment, the chimeric small molecule is according to

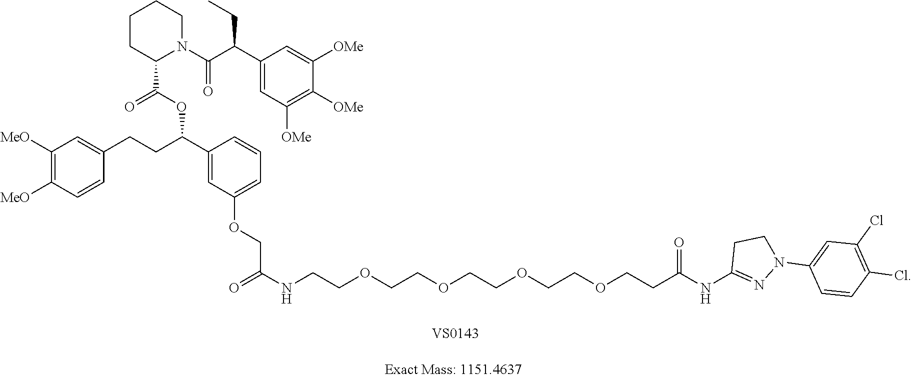 [SMILES: CC[C@H](C(=O)N1CCCC[C@H]1C(=O)O[C@@H](CCc1ccc(OC)c(OC)c1)c1cccc(OCC(=O)NCCOCCOCCOCCOCCC(=O)NC2=NN(c3ccc(Cl)c(Cl)c3)CC2)c1)c1cc(OC)c(OC)c(OC)c1 ; "VS0143"]

In one example embodiment, the molecule is capable of activating FGFR1/mTOR/G-CSF signaling in a dose-dependent manner.

### EGFR

In one example embodiment, the protein binding moiety is an EGFR binding moiety. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer.

In one embodiment, the protein binding molecule is an EGFR binding molecule of the formula,

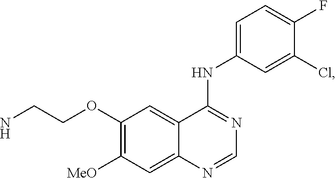 [SMILES: COc1cc2ncnc(Nc3ccc(F)c(Cl)c3)c2cc1OCCN]

or an analog thereof.

### HSP90

Heat Shock Protein 90 (Hsp90) is an ATP dependent molecular chaperone that with its co-chaperones modulates proteins involved in cell cycle control and signal transduction. Like many ATP dependent proteins, the protein undergoes a functional cycle that is linked to its ATPase cycle.

In an embodiment, the HSP90 binding molecule is

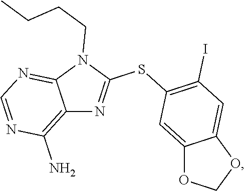 [SMILES: CCCCn1c(Sc2cc3c(cc2I)OCO3)nc2c(N)ncnc21]

or analog thereof.

Additional HSP90 binders include geldanamycin and derivatives thereof, including Tanspimycin (IC50 of 5 nM in cell free assay), according to the formula:

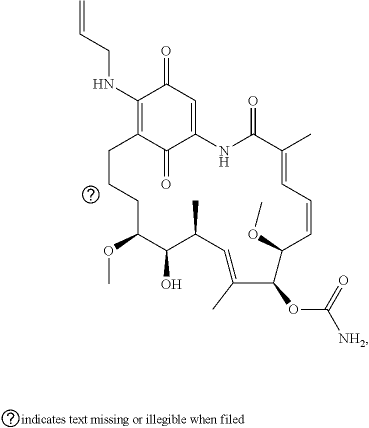 [SMILES: C=CCNC1=C2CCC[C@H](OC)[C@H](O)[C@@H](C)/C=C(\C)[C@H](OC(N)=O)[C@@H](OC)/C=C\C=C(/C)C(=O)NC(=CC1=O)C2=O]

Alvespimycin (IC50 of 62 nM in cell-free assay), according to the formula:

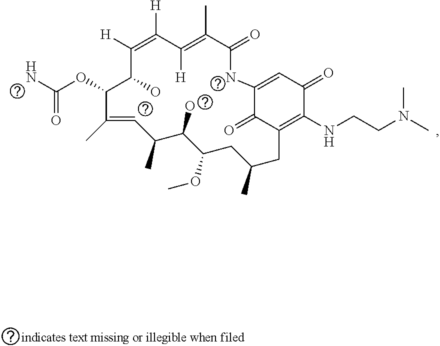

EC141 according to the formula:

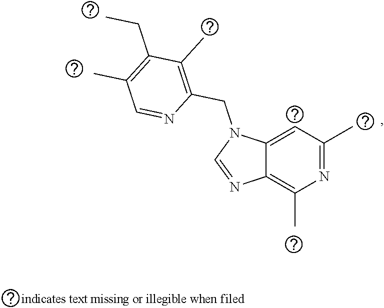

Novobiocin according to the formula:

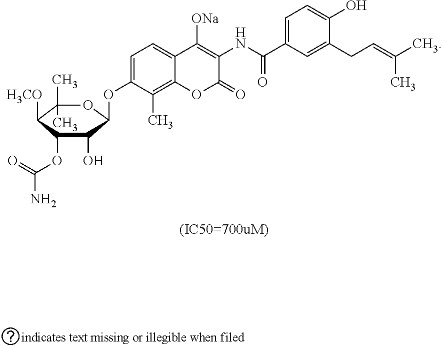

Novobiocin analogs can also be utilized and as described in Hall et al., J Med Chem. 2016 Feb. 11; 59(3): 925-933; doi: 10.1021/acs.jmedchem.5b01354, incorporated by reference, which can be used as a MAPK signaling disruptor.

### BTK

Bruton's Tyrosine Kinase (BTK) is a protein involved in multiple signaling cascades and is widely expressed in B cells. First, BTK is a cytoplasmic protein and thus available for interactions with cytoplasmic kinases, such as AMPK.

In an embodiment, the BTK binding molecule selected from the group consisting of:

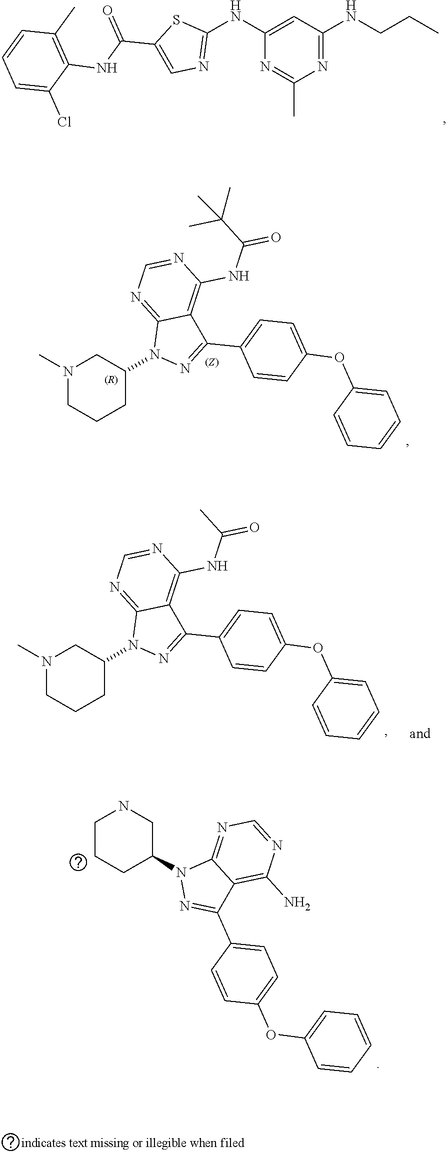

or an analog thereof.

### MDM2

In an embodiment, the target protein binding moiety is an MDM2 binding moiety according to

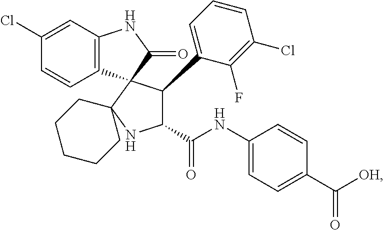 [SMILES: O=C(O)c1ccc(NC(=O)[C@@H]2NC3(CCCCC3)[C@@]3(C(=O)Nc4cc(Cl)ccc43)[C@H]2c2cccc(Cl)c2F)cc1]

or a derivative or analog thereof.

### BRD4

In an embodiment, the target protein binding moiety is a BRD4 binding moiety selected from the group consisting of

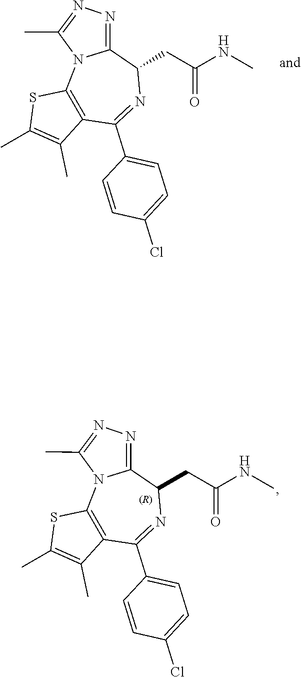

or an analog thereof.

### FGFR1

In one example embodiment, the target protein binding moiety inhibits FGFR1 fusion proteins. In one example embodiment, the FGFR 1 fusion protein inhibitor is Dovitinib, also known as TKI258, according to the formula

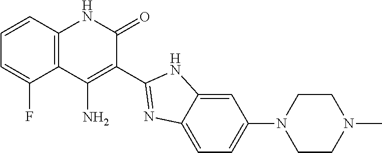 [SMILES: CN1CCN(c2ccc3nc(-c4c(N)c5c(F)cccc5[nH]c4=O)[nH]c3c2)CC1]

### PtpA, PtpB

In one example embodiment, target protein binding moiety is a PtpA binding moiety is according to the formula

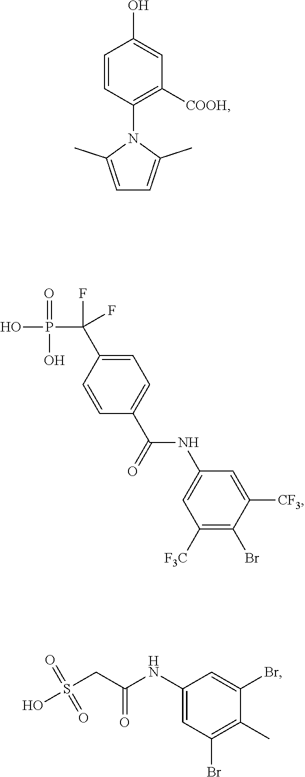

or any derivatives thereof.

In preferred embodiments, the PtpB binding moiety is according to the formula

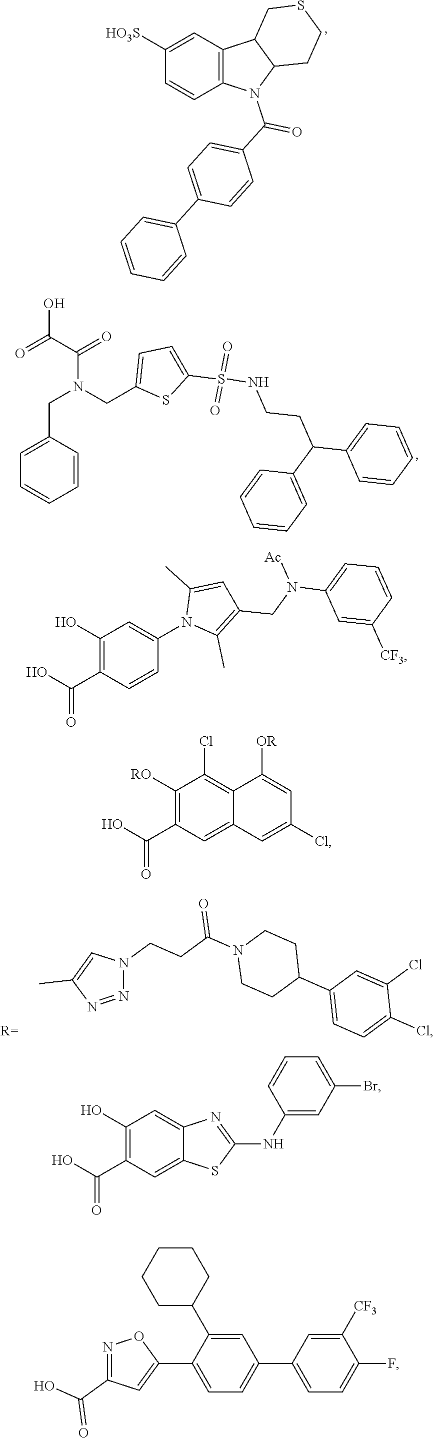

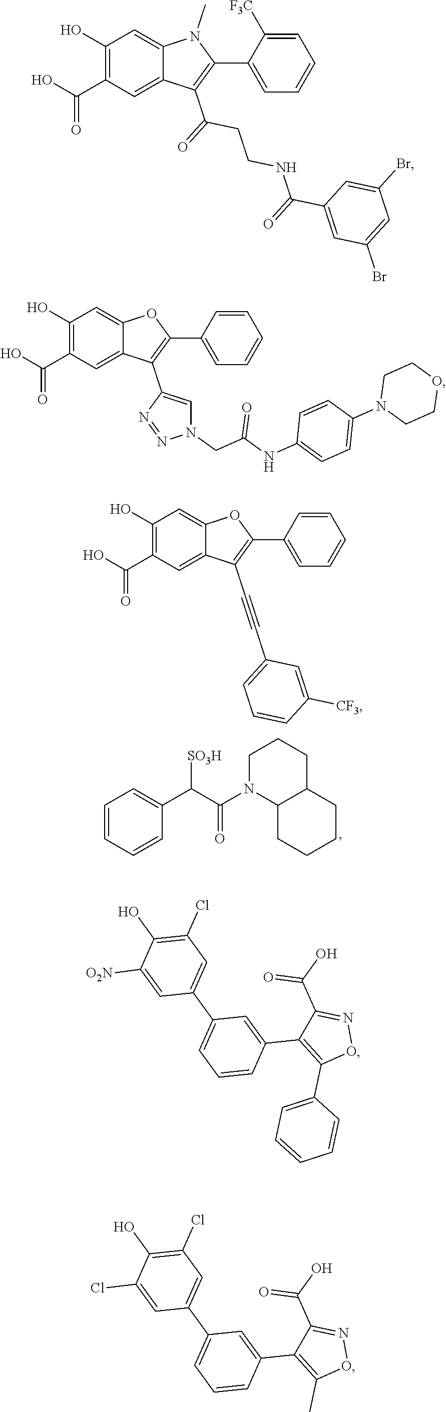

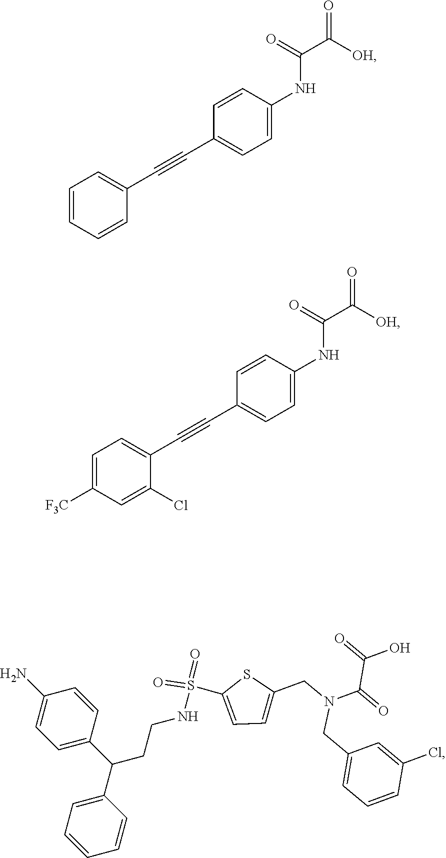 [SMILES: O=C(O)C(=O)Nc1ccc(C#Cc2ccccc2)cc1 ; O=C(O)C(=O)Nc1ccc(C#Cc2ccc(C(F)(F)F)cc2Cl)cc1 ; Nc1ccc(C(CCNS(=O)(=O)c2ccc(CN(Cc3cccc(Cl)c3)C(=O)C(=O)O)s2)c2ccccc2)cc1]

or any derivatives thereof.

### SapM

In one example embodiment, the target protein binding moiety is a SapM binding moiety. In an example embodiment, the SapM binding moiety contains a trihydroxy-benzene group. In an example embodiment, the SapM binding moiety comprises of a benzylidenemalononitrile scaffold. In one example embodiment, the SapM binding moiety has the formula:

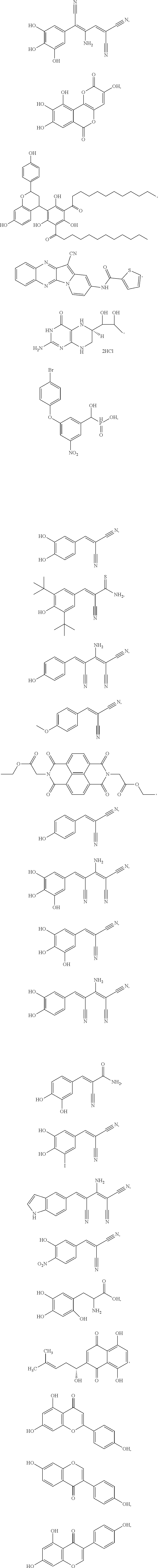

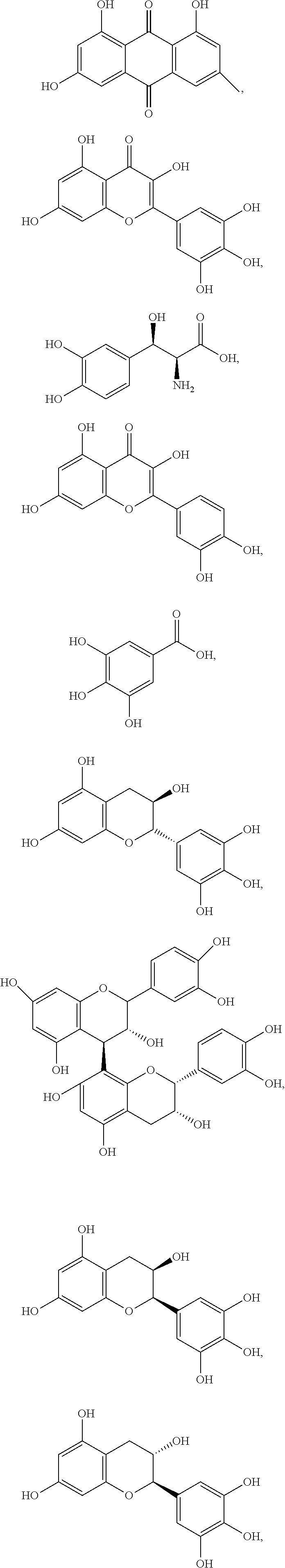

or any derivatives thereof. In one example embodiment the SapM binding moiety is L-ascorbic acid (L-AC) and 2-phospho-L-ascorbic acid (2P-AC).

### UMPK

In one example embodiment, the target binder is a M. tb kinase inhibitor. In an example embodiment, the M. tb kinase inhibitor is a UMPK inhibitor, and any derivative thereof identified in US Patent Application US US20090209022, herein incorporated by reference.

### Colistin

In preferred embodiments, the PsA associated target protein binding moiety is Colistin, which has the formula:

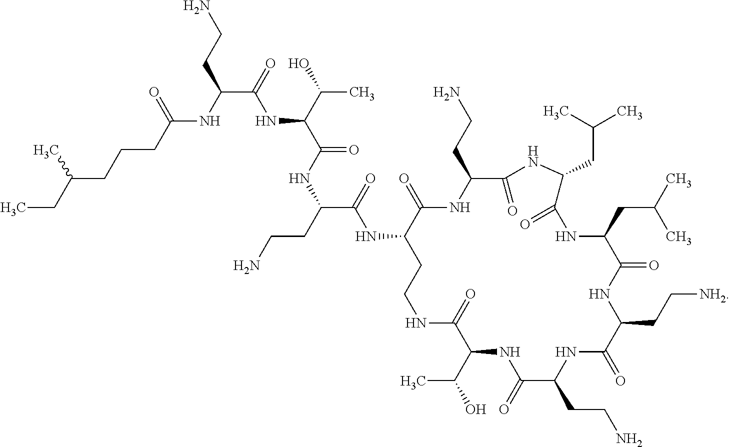 [SMILES: CCC(C)CCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]

### Bio-Orthogonal Group

The chimeric small molecules disclosed herein may further comprise a biorthogonal group. A chimeric small molecule may be configured to include a bio-orthogonal group as a device to remove the modifying polypeptide binding moiety from the target modifying polypeptide. This occurs when a coupling molecule, selected to react with the biorthogonal molecule, is introduced into the system containing the modifying polypeptide bound chimeric small molecule and bonds to the bio-orthogonal group. As a result, the modifying polypeptide binding molecule is no longer operable and cannot bind to the target modifying polypeptide. In one example embodiment, the modifying polypeptide binder comprises a bio-orthogonal group. In one example embodiment, the modifying polypeptide binder is modified to contain a bio-orthogonal group. Bio-orthogonal chemistry comprises chemical reactions carried out in a biological environment without reacting with endogenous systems, such as functional groups. Bio-orthogonal groups comprise moieties capable of bio-orthogonal chemistry. Non-limiting examples of bio-orthogonal groups include tetrazines, triazines, cyclooctenes, cyclopropenes and diazo groups.

In one example embodiment, the bio-orthogonal group comprises one of

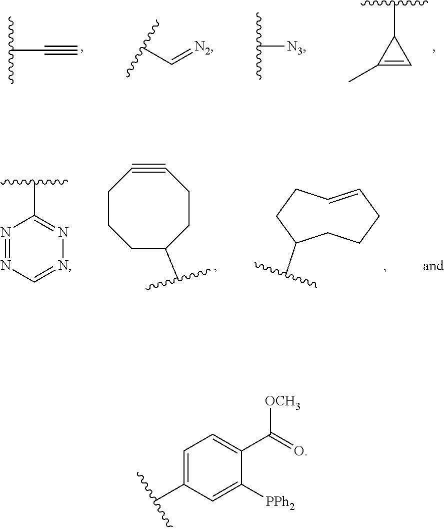

### Example Chimeric Small Molecules

Chimeric small molecules (e.g., bifunctional molecules) may be assembled using any combination of the above modifying binding moieties, electrophilic reactive linkers, and target binding moieties. The following description provides, by way of reference only, certain chimeric small molecules that can be generated according to the design principles and examples moieties provided above. In one aspect, a bifunctional molecule comprises an electrophilic reactive linker as described herein, and a target polypeptide binding moiety attached to the electrophilic reactive linker on one end and a modifying polypeptide binding moiety attached to the linker on the opposite end. In example embodiments, the target polypeptide binding moiety is a polypeptide to which the modifying moiety is to be attached and modified by the modifying moiety.

In example embodiments, the electrophilic reactive linker connects the functional moieties of a proteolysis targeting chimeras (PROTAC), deubiquitinase-targeting chimeras (DUBTACs), Pho-repressive complex (PhoRC), dephosphorylation targeting chimera (DEPTAC), phosphorylation targeting chimeras (PhosTAC), acetylation tagging system (AceTAG), regulated induced proximity targeting chimeras (RIPTAC), Transcriptional/Epigenetic Chemical Inducers of Proximity (TCIP), Autophagy-Targeting Chimera (AUTAC), or Lysosome Targeting Chimeras (LYTAC). See e.g., Peng, Y. et al., Targeted Protein Posttranslational Modifications by Chemically Induced Proximity for Cancer Therapy. Journal of Biological Chemistry, 2023, 299, 104572.

### Proteolysis Targeting Chimeras (PROTAC)

In an example embodiment, the functional moieties of a PROTAC are connected by an electrophilic reactive group linker. A PROTAC is a bifunctional molecule comprising of a moiety targeting a protein of interest and a moiety capable of binding to, for example, a ubiquitin ligase connected by a linker. See e.g., Pettersson, M.; Crews, C. M. PROteolysis TArgeting Chimeras (PROTACs)—Past, Present and Future. Drug Discovery Today: Technologies, 2019, 31, 15-27, hereby incorporated by reference. An example of an electrophilic reactive linker connecting the two moieties of a PROTAC can be understood through FIG. 1 of Pettersson et al. FIG. 1 highlights the linkers connecting various PROTACs. One of ordinary skill in the art could substitute the linkers described in Pettersson et al. with the electrophilic reactive linkers described herein using known methods in the art to achieve an electrophilic reactive linker PROTAC. Petterson et al. notes the linker composition, length, and attachment points can be advantageously altered to adjust the degree of selectivity. Accordingly, a PROTAC could be modified to include an electrophilic reactive linker described herein without inhibiting its function.

### Deubiquitinase-Targeting Chimeras (DUBTACs)

In an example embodiment, the functional moieties of a DUBTAC are connected by an electrophilic reactive group linker. A DUBTAC is a bifunctional molecule comprising of a moiety targeting a protein of interest and a moiety capable of binding to a deubiquitinase connected by a linker. See e.g., Henning, N. J.; et al. Deubiquitinase-Targeting Chimeras for Targeted Protein Stabilization. Nature Chemical Biology, 2022, 18, 412-421, hereby incorporated by reference. In example of an electrophilic reactive linker connecting the two moieties of a DUBTAC can be understood through FIG. 3 of Henning et al. FIG. 3 highlights the linkers connecting various DUBTACs. One of ordinary skill in the art could substitute the linkers described in Henning et al. with the electrophilic reactive linkers described herein using known methods in the art to achieve an electrophilic reactive linker DUBTAC. Henning et al. demonstrated various linkers can be used in the DUBTAC without inhibiting its function. Henning et al. altered composition, length, and stiffness and achieved varying degrees of performance without losing function. Accordingly, a DUBTAC could be modified to include an electrophilic reactive linker described herein without inhibiting its function.

### Pho-Repressive Complex (PhoRC)

In an example embodiment, the functional moieties of a PhoRC are connected by an electrophilic reactive group linker. A PhoRC is a bifunctional molecule comprising of a moiety targeting a protein of interest and a moiety capable of binding to a phosphatase connected by a linker. See e.g., Yamazoe, S.; et al. Heterobifunctional Molecules Induce Dephosphorylation of Kinases-A Proof of Concept Study. Journal of Medicinal Chemistry, 2019, 63, 2807-2813, hereby incorporated by reference. An example of an electrophilic reactive linker connecting the two moieties of a PhoRC can be understood through FIGS. 2, 3, and Scheme 1 of Yamazoe et al. FIGS. 2, 3, and Scheme 1 show the linkers connecting various PhoRCs. One of ordinary skill in the art could substitute the linkers described in Yamazoe et al. with the electrophilic reactive linkers described herein using known methods in the art to achieve an electrophilic reactive linker PhoRC. Yamazoe et al. notes linkers were designed for be sufficient to bind both the protein of interest and phosphatase. Accordingly, a PhoRC could be modified to include an electrophilic reactive linker described herein without inhibiting its function.

### Dephosphorylation Targeting Chimera (DEPTAC)

In an example embodiment, the functional moieties of a DEPTAC are connected by an electrophilic reactive group linker. A DEPTAC is a multi-functional peptide comprising of a moiety targeting a protein of interest, such as a tau proteins, and a moiety capable of binding to a phosphatase, for example, protein phosphatase 2A (PP2A) connected by a linker and further comprising a cell penetrating motif. See e.g., Zheng, J. et al. A Novel Dephosphorylation Targeting Chimera Selectively Promoting Tau Removal in Tauopathies. Signal Transduction and Targeted Therapy, 2021, 6, hereby incorporated by reference. An example of an electrophilic reactive linker connecting the two moieties of a DEPTAC can be understood through FIG. 1 of Zheng et al. FIG. 1 highlights the linker connecting a DEPTAC. One of ordinary skill in the art could substitute the linker described in Zheng et al. with the electrophilic reactive linkers described herein using known methods in the art to achieve an electrophilic reactive linker DEPTAC. Zheng et al. notes the purpose of the linker was to add flexibility. Accordingly, a DEPTAC could be modified to include an electrophilic reactive linker described herein without inhibiting its function.

### Phosphorylation Targeting Chimeras (PhosTAC)

In an example embodiment, the functional moieties of a PhosTAC are connected by an electrophilic reactive group linker. A PhosTAC is a bifunctional molecule comprising of a moiety targeting a protein of interest and a moiety capable of binding to, for example, a ubiquitin ligase connected by a linker. See e.g., Chen, P.-H.; et al. Modulation of Phosphoprotein Activity by Phosphorylation Targeting Chimeras (PhosTACs). ACS Chemical Biology, 2021, 16, 2808-2815, hereby incorporated by reference. An example of an electrophilic reactive linker connecting the two moieties of a PhosTAC can be understood through FIG. 1 of Chen et al. FIG. 1 highlights the various linkers connecting PhosTACs. One of ordinary skill in the art could substitute the linkers described in Chen et al. with the electrophilic reactive linkers described herein using known methods in the art to achieve an electrophilic reactive linker PhosTAC. Chen et al. notes the linker composition and length can be advantageously altered to adjust the degree of selectivity. Accordingly, a PhosTAC could be modified to include an electrophilic reactive linker described herein without inhibiting its function.

### Acetylation Tagging System (AceTAG)

In an example embodiment, the functional moieties of an AceTAG are connected by an electrophilic reactive group linker. An AceTAG is a bifunctional molecule comprising of a moiety targeting a protein of interest and a moiety capable of binding to an acetylase enzyme connected by a linker. See e.g., WO 2022187633 A1, hereby incorporated by reference. An example of an electrophilic reactive linker connecting the two moieties of a PROTAC can be understood through FIG. 2 of WO 2022187633 A1. FIG. 2 highlights the various linkers connecting AceTAGs. One of ordinary skill in the art could substitute the linkers described in WO 2022187633 A1 with the electrophilic reactive linkers described herein using known methods in the art to achieve an electrophilic reactive linker AceTAG. WO 2022187633 A1 notes the linker composition and length can be advantageously altered to adjust the degree of selectivity. Accordingly, an AceTAG could be modified to include an electrophilic reactive linker described herein without inhibiting its function.

### Regulated Induced Proximity Targeting Chimeras (RIPTAC)

In an example embodiment, the functional moieties of a RIPTAC are connected by an electrophilic reactive group linker. A RIPTAC is a bifunctional molecule comprising of a moiety targeting a protein of interest and a moiety capable of binding to pan-expressed protein essential for cell survival. See e.g., Raina, K. et al. Regulated Induced Proximity Targeting Chimeras (RIPTACs): A Novel Heterobifunctional Small Molecule Therapeutic Strategy for Killing Cancer Cells Selectively, 2023; Liu, J. O. Targeting Cancer with Molecular Glues. Science, 2023, 381, 729-730; Schulze, C. J.; et al. Chemical Remodeling of a Cellular Chaperone to Target the Active State of Mutant KRAS. Science, 2023, 381, 794-799, each of which are hereby incorporated by reference. An example of an electrophilic reactive linker connecting the two moieties of a RIPTAC can be understood through FIG. 2 of Raina et al. FIG. 2 highlights the linkers connecting various RIPTACs. One of ordinary skill in the art could substitute the linkers described in Raina et al. with the electrophilic reactive linkers described herein using known methods in the art to achieve an electrophilic reactive linker RIPTAC. Raina et al. demonstrates varying linker composition and length can be advantageously altered to adjust the degree of selectivity. Accordingly, a RIPTAC could be modified to include an electrophilic reactive linker described herein without inhibiting its function.

### Transcriptional/Epigenetic Chemical Inducers of Proximity (TCIP)

In an example embodiment, the functional moieties of a TCIP are connected by an electrophilic reactive group linker. A TCIP is a bifunctional molecule comprising of a moiety targeting a gene of interest and a moiety capable of binding to a transcription factor connected by a linker. See e.g., Gourisankar, S. et al. Rewiring Cancer Drivers to Activate Apoptosis, 2022, hereby incorporated by reference. An example of an electrophilic reactive linker connecting the two moieties of a TCIP can be understood through FIG. 2 of Gourisankar et al. FIG. 2 highlights the linkers connecting various TCIPs. One of ordinary skill in the art could substitute the linkers described in Gourisankar et al. with the electrophilic reactive linkers described herein using known methods in the art to achieve an electrophilic reactive linker TCIP. Gourisankar et al. notes the linker composition and length can be advantageously altered to adjust the degree of selectivity. Accordingly, a TCIP could be modified to include an electrophilic reactive linker described herein without inhibiting its function.

### Autophagy-Targeting Chimera (AUTAC)

In an example embodiment, the functional moieties of an AUTAC are connected by an electrophilic reactive group linker. An AUTAC is a bifunctional molecule comprising of a moiety targeting a protein of interest and a moiety capable of binding to a degradation tag (e.g., guanine tag) connected by a linker. See e.g., Takahashi, D.; et al. AUTACs: Cargo-Specific Degraders Using Selective Autophagy. Molecular Cell, 2019, 76, 797-810.e10, hereby incorporated by reference. An example of an electrophilic reactive linker connecting the two moieties of a AUTAC can be understood through FIGS. 2, 3, and 5 of Takahashi et al. FIGS. 2, 3, and 5 highlight the various linkers connecting various AUTACs. One of ordinary skill in the art could substitute the linkers described in Takahashi et al. with the electrophilic reactive linkers described herein using known methods in the art to achieve an electrophilic reactive linker AUTAC. Accordingly, a AUTAC could be modified to include an electrophilic reactive linker described herein without inhibiting its function.

### Lysosome Targeting Chimeras (LYTAC)

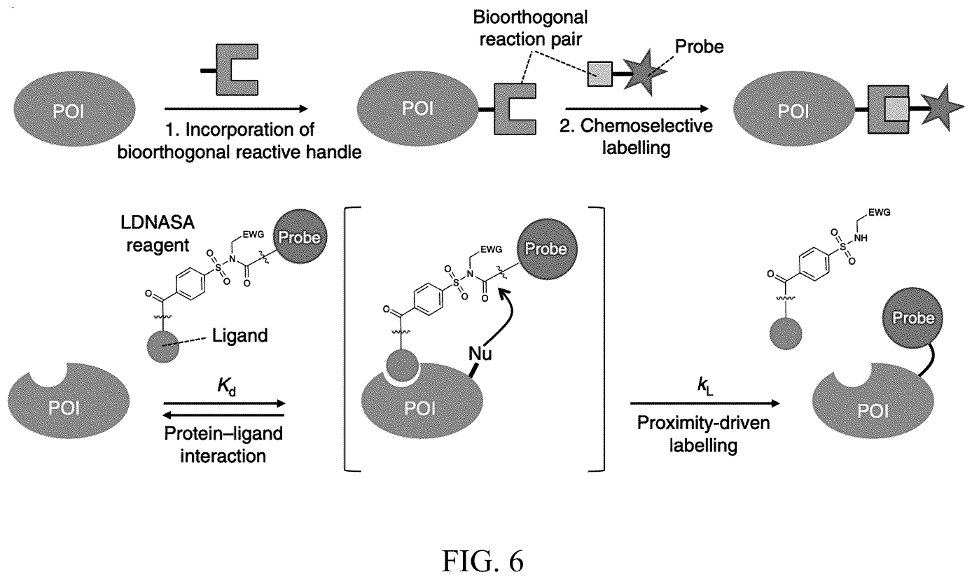

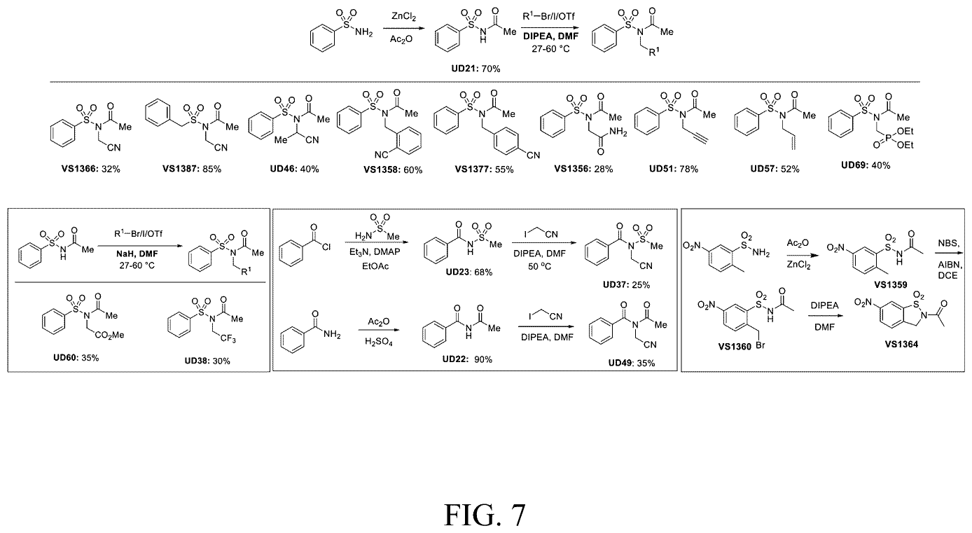

In an example embodiment, the functional moieties of a LYTAC are connected by an electrophilic reactive group linker. A LYTAC is a bifunctional molecule comprising of a moiety targeting an antibody or small molecule and a moiety capable of binding to plasma membrane-associated or secreted proteins connected by a linker. See e.g., Ahn, G.; et al. LYTACs That Engage the Asialoglycoprotein Receptor for Targeted Protein Degradation. Nature Chemical Biology, 2021, 17, 937-946, hereby incorporated by reference. An example of an electrophilic reactive linker connecting the two moieties of a LYTAC can be understood through FIG. 1 and Extended Data FIGS. 6 and 7 of Ahn et al. FIG. 1 and Extended Data FIGS. 6 and 7 show the linkers connecting various LYTACs. One of ordinary skill in the art could substitute the linkers described in Ahn et al. with the electrophilic reactive linkers described herein using known methods in the art to achieve an electrophilic reactive linker LYTAC. Accordingly, a LYTAC could be modified to include an electrophilic reactive linker described herein without inhibiting its function.

### Methods of Use

In another aspect, chimeric small molecules as described above may be used in methods to endow new functions to cellular modifying polypeptides or to regulate the activity of cellular modifying polypeptides. The chimeric small molecules find use for treatment in a variety of diseases and disorders. In an example embodiment, a target binding moiety can bind to a target of interest, preferably localizing in a region of a target of interest, allowing the protein to which the protein binding moiety is bound to modify a target. Exemplary applications include use in rewiring of cellular signaling. See, Lim et al., Nat Rev Mol Cell Biol 2010, 11(6), 393-403. For example, cell signaling can be addressed by appending phosphoryl groups to specific signaling protein of interest with dose and temporal control to allow rewiring of kinase signaling pathways in disease or health. The chimeric small molecule systems herein may enable targeted degradation of a target where phosphorylation sites are targets that recruit ubiquitin ligase and signal degradation. See, Toure et al., Angewandte Chemie (Inter'l ed. In English) 2016, 55(6), 1966-73. Similarly, preventing protein aggregation can aid in treatment in cancer treatment approaches. As described herein, addition of negatively charged phosphoryl groups using the chimeric small molecules on a protein prone to aggregation may increase solubility and reduce self-aggregation. Guo et al., FEBS Letters, 2005, 579 (17), 3574-3578; Zhang et al., Protein Expression and Purification 2004 36(2) 207-216. Exemplary embodiments comprising methods of treatment of kinasopathies are also provided. Exemplary embodiments further include regulation of nucleotide binding proteins, which may include use with orthogonally tagged nucleases such as Cas, and phosphorylation of transcription factors to affect binding. In one example embodiment, the invention described herein relates to a method for therapy in which cells are modified ex vivo by the chimeric small molecules to modify at least one target substrate, with subsequent administration of the edited cells to a patient in need thereof.

### Methods of Modifying Target Substrates

The chimeric small molecules disclosed herein can be utilized in methods of modifying a target substrate. Methods of modifying the target substrate can include generating a repurposed/reprogrammed cellular protein by delivering a chimeric small molecule, as described herein. In an example embodiment, the chimeric small molecules can be used to inhibit nucleotide binding proteins, inhibit oncogenic kinases, generate neo-antigens to evoke an immune response, as molecule prosthetics of kinasopathies, treatment of pathogens and induction of receptor tyrosine kinase signaling.

Methods of modifying a substrate are provided, which may be in a cell. In one example embodiment, a chimeric small molecule as described herein is introduced. In one example embodiment, the modifying comprises inducing post-translational modification of a target protein. In one example embodiment, the post-translational modification is phosphorylation. The method comprises administering to cell or cell population a chimeric small molecule. Methods of modifying the target substrate can include contacting the target substrate with a chimeric small molecule, e.g. bifunctional molecule, of the present invention. Contacting can allow for bonding to, or association with the target substrate, or to a molecule in proximity to a target substrate. Modification may be by inducing a conformational change via binding, changing structural stability, phosphorylation of a target, or via another mechanisms that affects the behavior of the target substrate. By way of example, activation or inactivation of the target substrate via the binding of the chimeric small molecule results in modification of the target substrate one or more new modification sites that would otherwise remain unmodified when the chimeric small molecule is not bound to the target substrate. In an aspect the methods comprise inducing phosphorylation of a target protein in the cell. The methods may comprise contacting a target substrate with the chimeric small molecule.

In an example embodiment, the chimeric small molecule can label the cellular protein with the target binding moiety for the target substrate via the electrophilic reactive group moiety. The electrophilic reactive group reacts with and bonds to a nucleophilic side chain on the cellular modifying polypeptide. Labelling of the cellular modifying polypeptide can allow for bonding to, or association with, the target substrate, or to a molecule in proximity to a target substrate facilitating modification of the target substrate. In one example embodiment, this approach allows utilization of protein inhibitor moieties, as well as activators and neutral binding molecules to induce target modification. Such modifying polypeptide binding moieties tethered with an electrophilic reactive group, e.g., a chemoselective electrophilic warhead, exhibits site-specific labeling of a side chain nucleophilic residue, e.g., nucleophilic side chain amino acid, proximal to the inhibitor binding site. Generally, labeling proximal to the inhibitor binding site refers to a reactive group at, within, or at a distance to the binding moiety binding site that allows the electrophilic reactive group to react at or near the time and/or space of the binding site of the binding moiety. The tethering of the electrophilic warhead can comprise a linker, bond, and/or exit vector or adapter which may, in some instances, In one aspect, the target substrate is not a natural substrate of the protein, or wherein activation of the protein by the binding moiety results in modification of the target substrate by the protein at one or more new modification sites that would otherwise remain unmodified by the protein when not activated by binding to the activator moiety. Modification may be by inducing a conformational change via binding, changing structural stability, phosphorylation of a target, or via other mechanisms that affects the behavior of the target substrate, e.g., removal of groups such as phosphatases, methyltransferases. Modifying can include the post-translational modification as disclosed herein, including, for example, phosphorylation, hydroxylation, acetylation, methylation, glycosylation, prenylation, amidation, eliminylation, lipidation, acylation, lipoylation, deacetylation, formylation, S-nitrosylation, S-sulfenylation, sulfonylation, sulfinylation, succinylation, sulfation, carbonylation, or alkylation. In one aspect, the methods comprise inducing phosphorylation of a target protein in or on the cell. The methods may comprise contacting a target substrate with the chimeric small molecule. In one example embodiment, the target substrate is in proximity to a kinase specific to the protein binding moiety of the molecule. Chimeric small molecules that induce phosphorylation can be optionally provided with adenosine monophosphate (AMP) or another molecule providing an additional phosphate group. Without being bound by theory, the addition of the AMP or other phosphate providing molecule can enhance phosphorylation.

In an example embodiment, inhibition of nucleotide-binding proteins may comprise inhibition binding of a CRISPR-Cas protein to a nucleic acid or transcription factors binding to DNA. Thus, proteins that have been modified to comprise a binding domain that can be targeted by an orthogonal tag, e.g., Cas9 comprising a FKBP binding domain, can be inhibited by the use of small molecules comprising an orthogonal tag, such as a dTAG. Sequence specific modular adaptors consisting of a DNA-binding protein and a self-ligating protein tag can be utilized. See, e.g., Nguyen et al., Rational design of a DNA sequence-specific modular protein tag by tuning the alkylation kinetics, Chem Sci., 40 (2019) doi: 10.1039/C9SC02990G. Similarly, nucleotide binding may be modified via the modification of transcription factors with the chimeric small molecules. Because post-translation phosphorylation of transcription factors might be necessary for direct binding interactions or a conformational change in a transcription factor, thereby leading to, activating, or inhibiting gene transcription, methods of modification of transcription factors are provided. In an example embodiment, methods of use can comprise eliciting an immune reaction, creation of an autoantigen, and target deactivation. In an exemplary embodiment, hyper-phosphorylation or neo-phosphorylation of a target protein may result in immune recruitment to a target, for example via trigger display of neo-eptiopes and T-cell attack on cells displaying the epitopes. In one application, the small molecules disclosed herein are utilized in human leukocyte antigen (HLA) display and immune response. Neo-phosphorylation to elicit an immune response can find use in cancer immunotherapy approaches. In an exemplary approach, a kinase is selected for the phosphorylation of p53, for example, at Ser33, Ser315 and/or Thr82. This phosphorylation leads to subsequent binding and conformational changes which leads to activation as a transcription factor. See, e.g. Ryan and Vousden, Nature, 419 (2002). Thus, design of a molecule comprising a binding moiety for a kinase that phosphorylates or dephosphorylates along with a target for p53 can allow control of nucleotide binding based on desired conformation of the transcription factor. See also, e.g. Mattiske T, Tan M H, Dearsley O, Cloosterman D, Hii C S, Gécz J, et al. (2018) Regulating transcriptional activity by phosphorylation: A new mechanism for the ARX homeodomain transcription factor. PLOS ONE 13(11): e0206914. doi:10.1371/journal.pone.0206914.

### Kinasopathies

Treatment of kinasopathies are also contemplated, see, generally, Lahiry et al., Nature Reviews Genetics, 2011, with Table 1 disclosure of inherited kinasopathies incorporated herein by reference. Accordingly, for kinasopathies that have a loss of function, a chimeric small molecule according to the invention can recruit a working kinase to provide the lost function. See, e.g. Lahiry et. al, Nature Reviews Genetics volume 11, pages 60-74 (2010) (discussing various germline disorders and cancers related to kinase dysfunction), incorporated herein by reference, in particular Supplementary Table 1 of inherited kinasopathies and Supplementary Table 2 of kinases associated with cancer. In an exemplary embodiment, Src family protein tyrosine kinases (SFKs) are stabilized in active conformation by phosphorylation of a conserved Y_{A} in the active A-loop conformation. By targeting an SFK for modification, e.g. phosphorylation at the A-loop, treatment of aberrant SFK can address kinasopathies associated with the SFK, e.g. ALL, CML. See, e.g., Mechanism of Drug-Resistance in Kinases, Expert Opin Investig Drugs. 2011 February; 20(2): 153-208; doi: 10.1517/13543784.2011.546344.

Methods of Use with Kinase Inhibitor Binding Moiety

In one example embodiment, the method comprises generating a reprogrammed cellular kinase by delivering a chimeric small molecule of the formula A-L₁-E-B or A-L₁-E-L₂-B, wherein A is an protein binding moiety specific for the cellular protein to be repurposed/reprogrammed; B is a target binding moiety specific for the target substrate to be modified; L₁ and L₂ is a linker; and E is an electrophilic reactive group whereby the chimeric small molecule labels the cellular protein with the target binding moiety for the target substrate; and modifying the target substrate by binding of the repurposed/reprogrammed protein to the target substrate via the target binding moiety, whereby the repurposed/reprogrammed cellular protein introduces one or more modifications to the target substrate. In one example embodiment, the protein binding moiety has a half-life about 2, 3, 4, 5, 6 or 7 times less than a half-life of the protein to be repurposed/reprogrammed. In one example embodiment, the protein to be reprogrammed is an oxidoreductase, transferase, hydrolase, lyase, isomerase, ligase, or translocase. In one example embodiment, an inhibitor is a protein binding moiety. In one example embodiment, the protein to be repurposed/reprogrammed is a kinase and the protein binding moiety is a kinase inhibitor. In one example embodiment, the kinase inhibitor is a ‘promiscuous’ kinase inhibitor. In one example embodiment, the method comprises administering a coupling molecule thereby quenching the inhibitory activity of the protein inhibitor. In one example embodiment, the coupling molecule is one or more of an aldehyde, alkene, alkyne, strained alkyne, cyclooctyne, trans-cyclooctene, cyclopropene, oxanorbornadiene, norbornene, phosphine, electron-rich dienophile, isonitrile, isocyanopropanoate, tetrazole, 2-acylboronic acid, or any derivative thereof. In one example embodiment, the cyclooctyne derivative comprises dibenzocyclooctyne, biarylazacyclooctynone, or dimethoxyazacyclooctyne. In one example embodiment, the method comprises a strained alkyne comprising a bicyclononyne or dioxabiaryldecyne.

In one example embodiment, the method comprises a chimeric small molecule wherein the protein binding moiety has a half-life about 2, 3, 4, 5, or 6 times less than a half-life of the protein to be reprogrammed. In an example embodiment, the protein to be reprogrammed is an oxidoreductase, transferase, hydrolase, lyase, isomerase, ligase, or translocase. In an example embodiment, the kinase binding moiety is an inhibitor. In an example embodiment, the protein to be reprogrammed is a kinase and the kinase binding moiety is a kinase inhibitor. In an example the kinases inhibitor is a promiscuous kinase inhibitor.

In one example embodiment, the method comprises a chimeric small molecule wherein the kinase binding moiety contains a bio-orthogonal group capable of reacting with and bonding to a coupling molecule. In an example embodiment, the method further comprises administering a coupling molecule. The coupling small molecule is administered to react with the bio-orthogonal group on the chimeric small molecule and, as a result, quench the kinase inhibitor from binding to the kinase. In an example embodiment, the coupling molecule is one or more of an aldehyde, alkene, alkyne, strained alkyne, cyclooctyne, trans-cyclooctene, cyclopropene, oxanorbornadiene, norbornene, phosphine, electron-rich dienophile, isonitrile, isocyanopropanoate, tetrazole, 2-acylboronic acid, or any derivative thereof. In an example embodiment, the cyclooctyne derivative comprises dibenzocyclooctyne, biarylazacyclooctynone, or dimethoxyazacyclooctyne. In one example embodiment, the strained alkyne comprises bicyclononyne or dioxabiaryldecyne.

In one example embodiment, the coupling molecule is co-administered with the chimeric small molecule. In an example embodiment, the coupling molecule is administered after the administration of the chimeric small molecule. In an example embodiment, the coupling molecule is administered within 24 hours, or within 12 hours, or within 11 hours, or within 10 hours, or within 9 hours, or within 8 hours, or within 7 hours, or within 6 hours, or within 5 hours, or within 4 hours, or within 3 hours, or within 2 hours, or within 1 hour, or within 30 minutes or less of administering the chimeric small molecule.

### Delivery of Coupling Molecules

In one aspect, the method comprises an additional step of administering or delivering a coupling molecule. The coupling molecule, as previously described, reacts with a bio-orthogonal group on the protein targeting moiety. This reaction suppresses binding of the binding moiety to the protein. When utilized with a chimeric small molecule comprising an electrophilic reactive group, the protein binding moiety may be released from the chimeric small molecule, and the coupling molecule may bind to the biorthogonal group of the modifying polypeptide binding moiety, thereby preventing the modifying polypeptide binding moiety from further binding the modifying polypeptide. In one example embodiment, the coupling molecule is utilized with a modifying polypeptide binding moiety that is an inhibitor of the protein.

The coupling molecule may be administered in any pharmaceutical formulation, effective amount, and dosage form previously described. The coupling molecule may be delivered using any previously described method or administered with any co-therapies or combinations and as described herein. The chimeric small molecule and the coupling molecule may by delivered or administered concurrently or sequentially. The concurrent delivery of the coupling molecule and chimeric small molecule may occur within the same delivery method or with a separate delivery method. The concurrent but separate delivery of the coupling small molecule may be the same type of delivery method or a different type of delivery method previously described. Sequential delivery of the coupling molecule may occur with the same type of delivery method or different type of delivery method. Sequential delivery of the coupling molecule may occur within 24 hours, or within 12 hours, or within 11 hours, or within 10 hours, or within 9 hours, or within 8 hours, or within 7 hours, or within 6 hours, or within 5 hours, or within 4 hours, or within 3 hours, or within 2 hours, or within 1 hour, or within 30 minutes or less of administering the chimeric small molecule.

### Coupling Molecules

In one preferred embodiment, a coupling molecule is introduced to a system containing the chimeric small molecule. As the coupling molecule comes into contact with the chimeric small molecule bound to the target protein, it quenches the binding between the protein binding moiety and target protein. In one example embodiment, the coupling molecule is a molecule capable of undergoing a reaction with a biorthogonal molecule, which is a substituent of the protein binding moiety. The reaction results in the coupling molecule attaching to the protein binding moiety and, as a result, the protein binding moiety no longer binds to the protein. In one example embodiment, the coupling molecule can react with the bio-orthogonal moiety through an aldehyde/ketone-nucleophile reaction, dipolar cycloaddition, phosphine ligation, Diels-Alder cycloaddition, [4+1] cycloaddition, nitrile imine-alkene reaction, or 2-acylboronic acid condensation, or any other bio-orthogonal reaction.

In one example embodiment, the coupling molecule and bio-orthogonal moiety couple through a aldehyde/ketone-nucleophile condensation. Classily, an aldehyde couples with an amine group such as alkoxyamine or hydrazine, for example. While intracellular metabolites contain aldehydes and ketones, this approach is effective on the cell surface. In one preferred embodiment, the coupling molecule is an aldehyde.

In one example embodiment, the coupling molecule and bio-orthogonal moiety couple through a dipolar cycloaddition. Dipolar cycloadditions typically occur between azides and alkynes and either in the presence or absence of copper. In the case of copper free dipolar cycloadditions, the alkyne is strained to facilitate the reaction. In most cases, the strained alkyne is cyclooctyne or any derivative thereof. Non-limiting examples of cyclooctynes include: dibenzocyclooctyne, biarylazacyclooctynone, and dimethoxyazacyclooctyne. In one example embodiment, the coupling molecule is an alkyne. In an example embodiment the coupling molecule is a strained alkyne. In one preferred embodiment, the coupling molecule is cyclooctyne. While it is understood any strained alkyne may be used other non-limiting examples include bicyclononyne, dioxabiaryldecyne, and any derivative thereof.

The dipolar cycloaddition may also comprise a reaction between oxanorbornadiene and an azide. In this case, after the cycloaddition between the oxanorbornadiene and azide, a spontaneous retro-Diels Alder reaction occurs generating a triazole and furan. In one example embodiment the coupling molecule is oxanorbornadiene or any derivative thereof.

The dipolar cycloaddition may also comprise the reaction between norbornene and a nitrile oxide. In one example embodiment, the coupling molecule is norbornene. The coupling molecule may also perform a dipolar cycloaddition with another dipolar molecule such as a nitrone, (imino) syndone, or 1,3-dithiolium-4-olate and would comprise of the counterpart unsaturated hydrocarbon.

In one example embodiment, the coupling molecule and bio-orthogonal moiety couple through a phosphine ligation, or interchangeably referred to as the Staudinger ligation. A phosphine ligation typically occurs between an azide and phosphine typically forming a phosphine oxide and a stable amide linkage or, when electron deficient aromatic azides are used, forming an iminophosphorane. In one example embodiment, the coupling molecule is a phosphine or any derivative thereof. Phosphine ligations may also comprise a cyclopropene in place of the azide. Non-limiting examples of cyclopropane include: cyclopropenones, cyclopropenethiones, cyclopropenium ions.

In one example embodiment, the coupling molecule and bio-orthogonal moiety couple through a Diels-Alder cycloaddition. The reaction is an inverse electron-demand Diels-Alder and classically occurs between an electron-poor diene and an electron-rich dienophile. In one example embodiment, the coupling molecule is an electron-rich dienophile. The Diels-Alder cycloaddition may comprise a tetrazine ligation wherein a strained unsaturated hydrocarbon and a tetrazine or triazene couple to form a pyridazine. In one example embodiment, the coupling molecule is a strained unsaturated hydrocarbon. The unsaturated hydrocarbon may also be cyclic. Non-limiting example of strained, cyclic unsaturated hydrocarbons include cyclooctynes, trans-cyclooctenes, norbornenes, cyclopropenes, and azetines. In preferred embodiments, the coupling molecule is a cyclooctyne, trans-cyclooctene, or a derivative thereof.

In one example embodiment, the coupling molecule and bio-orthogonal moiety couple through a [4+1] cycloaddition. The reaction involves the coupling of an isonitrile with, classically, a tetrazine followed by a spontaneous retro-Diels Alder elimination. The conjugate of the reaction is more stable if the isonitrile is tertiary. However, less stable conjugates are formed when the isonitrile is primary or secondary. In one preferred embodiment, the coupling molecule is an isonitrile or any derivative thereof. In one example embodiment, the isonitrile is tertiary. In one preferred embodiment, the coupling molecule is isocyanopropanoate or any derivative thereof.

In one example embodiment, the coupling molecule and bio-orthogonal moiety couple through a nitrile imine-alkene cycloaddition. Classically, tetrazole is photolyzed to generate nitrile imine which readily couple with unsaturated hydrocarbons. The wavelength necessary for photolysis is dependent on the substituents of tetrazine. However, photolysis is not required if hydrazonoyl chlorides are present, which, at neutral pH, spontaneously generate nitrile imines from tetrazole. In one preferred embodiment the coupling molecule is an unsaturated hydrocarbon and is optionally introduced with a hydrazonoyl chloride.

In one example embodiment, the coupling molecule and bio-orthogonal moiety couple through a 2-acylboronic acid condensation. In this reaction, the boronic acid couples with an amine to form a stable diazaborine. In one preferred embodiment, the coupling molecule is 2-acylboronic acid or any derivative thereof. See e.g., Shieh P, Bertozzi C R. Design strategies for bioorthogonal smart probes. Org Biomol Chem. 2014; 12(46):9307-9320. doi:10.1039/c4ob01632g and Mike L. W. J., et al., Recent developments in bioorthogonal chemistry and the orthogonality within, Curr. Opin. Chem. Biol., 2021, 60, 79-88, herein incorporated by reference.

### Oncogenic Applications

In one example embodiment, the disease is associated with cancer. In particular, the disease is oncogenic. Many oncogenic targets are known and can be regulated by posttranslational modifications. See, e.g. Chen, L., Liu, S. & Tao, Y. Regulating tumor suppressor genes: post-translational modifications. Sig Transduct Target Ther 5, 90 (2020); doi:10.1038/s41392-020-0196-9. Exemplary post-translational modification types of proteins implicated in oncogenesis and their expression pattern are found in Table 1 of Sharma, et al., (2019). Post-Translational Modifications (PTMs), from a Cancer Perspective: An Overview. Oncogen 2(3): 12, specifically incorporated herein by reference.

The chimeric small molecules disclosed herein can be utilized in methods of treating cancer. Methods of treating cancer can include generating a repurposed/reprogrammed cellular protein by administering a chimeric small molecule, as described herein. The chimeric small molecule labels the cellular protein with an oncogenic modifying polypeptide binding moiety via the electrophilic reactive group moiety. The electrophilic reactive group reacts with and bonds to a nucleophilic side chain on the cellular protein. Labelling of the cellular protein can allow for bonding to, or association with, the oncogenic target protein, or to a molecule in proximity to the oncogenic protein facilitating modification of the target substrate. In one aspect, the methods comprise inducing phosphorylation of the oncogenic target protein in or on the cell. The methods may comprise contacting the oncogenic target protein with the chimeric small molecule. In one example embodiment, the oncogenic target protein is in proximity to a kinase specific to the protein binding moiety of the molecule. Chimeric small molecules that induce phosphorylation can be optionally provided with adenosine monophosphate (AMP) or another molecule providing an additional phosphate group. Without being bound by theory, the addition of the AMP or other phosphate providing molecule can enhance phosphorylation.

Methods of treating cancer are provided. The method of treating cancer comprises generating a reprogrammed cellular protein by administering to a subject in need thereof a chimeric small molecule of the formula: A-L₁-E-B or A-L₁-E-L₂-B, wherein A is a modifying polypeptide binding moiety; E is an electrophilic reactive group and B is an oncogenic target protein to be modified, whereby the chimeric small molecule labels the cellular protein with the target binding moiety for the target substrate; and modifying the oncogenic target protein by binding of the repurposed/reprogrammed protein to the target substrate via the target binding moiety, whereby the repurposed/reprogrammed cellular protein introduces one or more modifications to the target substrate. In one example embodiment, the target binding moiety is specific for KRAS, RAS, FKPB12^{F36V}, EGFR, HSP90, BTK, MDM2, BRD4, BCR-ABL, NF-kB, LDH-A, p53, GP73, MUC1, MUC16, CD44, GPCR, HMGB1, RIOK1, CHK1, UBE2F, HUR, PTEN, STAT-3, Osteopontin, EGFRs, AKT, DAPK1, Rho, Ubc9, FOXK2, HIC1, HER2, BRAF, BCL-2, CD117, (KIT), ALK, PI3K, Delta, DNMT1, or SMO. In one example embodiment, the cellular protein to be reprogrammed is a oxidoreductase, transferase, hydrolase, lyase, isomerase, ligase, translocase. In one example embodiment, the kinase binding moiety is a kinase inhibitor. In one example embodiment, the kinase inhibitor is specific for PK, PKC, AMPK, MAPK, EGFR, FGFR, NGFR, TrkA, ABL, BCKDK, CDK, PI3K, VEGFR, BRAF, MEK, AKT, ALK, BTK, FLT3, JAK2, AURKA, c-MET, DDR, FKBP, INSR, IKK, JNK, mTOR, PAK, PDK1, PDK2, PTK2/FAK, pyruvate kinases, RAC-α, RIPK, TYK2, SHP, aPKC, NOP, μ opioid receptor, δ opioid receptor, UMPK, SphK, or GSK-3. In one example embodiment, administering a coupling molecule thereby quenching the inhibitory activity of the kinase inhibitor.

Methods of treating a disease associated with aberrant KRAS signaling is provided, comprising administering a composition comprising a chimeric small molecule, the chimeric small molecule comprising the KRAS binding molecule and a kinase binding molecule of as described herein. In one example embodiment, the kinase binding molecule is a target for an kinase selected from the group consisting of: PK, PKC, AMPK, MAPK, EGFR, FGFR, NGFR, TrkA, ABL, BCKDK, CDK, PI3K, VEGFR, BRAF, MEK, AKT, ALK, BTK, FLT3, JAK2, AURKA, c-MET, DDR, FKBP, INSR, IKK, JNK, mTOR, PAK, PDK1, PDK2, PTK2/FAK, pyruvate kinases, RAC-α, RIPK, TYK2, SHP, aPKC, NOP, μ opioid receptor, δ opioid receptor, UMPK, SphK, or GSK-3. In an embodiment, the kinase binding molecule is an AMPK binding moiety. In one example embodiment, the KRAS is KRAS^{G12C}. In one example embodiment, the chimeric small molecule phosphorylates one or more residues on KRAS selected from the group consisting of Ser17, Ser39, Ser65, Ser106, Ser122, Ser136, Ser2, Thr2, Thr35, Thr50, Thr74, Thr87, Thr124, Thr127, Thr148.

In an example embodiment, a method of treating cancer in a cell is provided, comprising administering a chimeric small molecule of the present invention. In one example the small molecule comprises a P13K kinase binder, a linker, an electrophilic reactive group, and a p53 target binding moiety, e.g., based on idasanutlin. In one example embodiment, the molecule comprises a binder of P13K based on the inhibitor PIK108 that further optionally comprises bioorthogonal group, e.g. cyclopropenyl. The binding moiety PIK108 comprises a linker connected to the electrophilic reactive group, e.g. dibromophenyl benzoate. The electrophilic reactive group, in turn, is connected to the p53 protein target binding moiety, optionally via a linker. Upon binding to the PI3K kinase via PIK108, proximal lysines of the binding pocket of PI3K, and will react with the lysine-reactive group (e.g., dibromophenyl benzoate) and expel the kinase inhibitor, leaving the P13K tagged with the p53 binder. The kinase which is covalently labeled with the target binding moiety can then hyper and/or neo-phosphorylate the p53. Administration of a tetrazine coupling molecule can quench the cyclopropenyl biorthogonal group when displayed on the PI3K binding molecule, and deactivate the expelled kinase binding moiety.

Exemplary oncogenic fusion proteins that can be treated by the binding of a multimeric kinase include fusions associated with the ABL proteins. ABL proteins are non-receptor tyrosine kinases that are normally under well-orchestrated regulation. However, chromosome translocations that join the ABL genes with genes coding for other proteins give rise to various oncogenic fusion proteins (BCR-ABL, TEL-ABL, NUP214-ABL, etc.) that are prone to dimerization (or oligomerization) and subsequent autophosphorylation. Consequently, ABL kinase becomes constitutively active and lead to diseases such has chronic myeloid leukemia (CML), acute lymphocytic leukemia (ALL) and other myeloproliferative disorders. An example oncogenic fusion is BCR-ABL. This may be particularly true for kinases such as Abl which must form complexes to become active.

In example embodiments, the cancer is characterized by an oncofusion of a kinase, e.g. ABL-kinase. Oncogenic ABL fusion proteins are known in the art and implicated in a variety of proliferative disorders. Chromosome translocations occur joining the genes of ABL with genes coding for other proteins, giving rise to various proteins that are prone to dimerization (or oligomerization) and autophosphorylation, making the ABL kinase constitutively active and leading to myeloproliferative disorders. In example embodiments, the oncofusion is TEL-ABL or NUP214-ABL.

Translocation events in cancer have been shown to be associated with fusions involving ALK, BRAF, EGFR, FGFR1, 2 and 3, NTRK1, 2 and 3, PDGFRA, PRKCA and B, RAF1, RET, ROS1, FGR, MET, PIK3CA, and PKN1. Chimeric small molecules designed as molecular glue for targeting of the fusions using the design considerations for the small molecules as described herein. In particular, druggable kinases that engage in fusions include AKT3, ALK, BRAF, BRD4, CD74, EGFR, EML4, ERBB4, ESR1, FGFR2, FGFR3, JAK2, MET, NOTCH1, NRG1, NTRK1, NTRK3, NUP214-ABL1, PDGFRA, PDGFRB, PML-RARA, RAF1, RET, ROS1, TMPRSS2, and TRIM33-RET have also been identified.

Additional fusions that can be targeted with the chimeric small molecules taught herein include, but are not limited to, ACSM2B-NOTCH2, ACTG2-ALK, ACVR2A-AKT3, AFF3-TMPRSS2, AGGF1-RAF1, AGK-BRAF, AKAP13-NRG1, AKAP13-NTRK3, AKAP13-RET, AKAP7-ESR1, AKT3-ADSS, AKT3-CDC14A, AKT3-HEATR1, AKT3-PPP2R2A, AKT3-PTPRR, ALK-GALNT14, ALK-SCEL, ALK-STK39, AP3B1-BRAF, ARHGEF25-NTRK1, ARID2-TMPRSS2, ATAD2-ERBB4, ATF7IP-TMPRSS2, ATG7-BRAF, ATP1A1-NOTCH2, ATP1B1-NRG1, ATP2B4-ERBB4, B4GALT1-RAF1, BACE2-TMPRSS2, BAIAP2L1-MET, BCL2L11-BRAF, BCR-ABL1, BRAF-AP3B1, BRAF-ATG7, BRAF-CUL1, BRAF-DENND2A, BRAF-FAM114A2, BRAF-HIBADH, BRAF-MACF1, BRAF-MED4, BRAF-SND1, BRAF-SUGCT, BRD4-AKAP8L, BRD4-CC2D1A, BRD4-CSE1L, BRD4-CSN2, BRD4-CYP4F22, BRD4-GNAT1, BRD4-MFSD12, BRD4-NOTCH3, BRD4-PGLYRP1, BRD4-PGLYRP2, BRD4-SLC1A6, BRD4-ZC3H15, C8orf34-MET, CBR4-ERBB4, CCAR2-FGFR2, CCDC6-RET, CD74-ROS1, CDC27-BRAF, CDK12-JAK2, CDK2-ALK, CEL-NTRK1, CEP170-AKT3, CEP85L-ROS1, CHIC2-PDGFRA, CLCN6-RAF1, CLOCK-PDGFRA, CLTC-ROS1, CMTM8-RAF1, CUX1-BRAF, DANCR-PDGFRA, DLG5-RET, DLG5-TMPRSS2, DNM1-FGFR2, DOCK8-JAK2, DSTYK-BRAF, EGFR-ACADM, EGFR-C7orf72, EGFR-CHODL, EGFR-DYM, EGFR-GRB10, EGFR-GYG1, EGFR-INSL4, EGFR-LYST, EGFR-RCL1, EGFR-SEPT14, EGFR-SEPT14P24, EGFR-TEAD3, EGFR-VSTM2A, EIF5-NOTCH2, EML4-ALK, EML4-NTRK3, EPHB2-NTRK1, EPS15L1-BRD4, ERBB4-RBM33, ERBB4-SDCCAG8, ERBB4-SLC25A10, ERC1-RET, ERG-TMPRSS2, ESR1-ASPH, ESR1-BNC2, ESR1-GNAS, ESR1-MYCT1, ESR1-, DE7B, ESR1-POLH, ESR1-POLR2E, ESR1-SIM1, ESR1-SYNE1, ESR1-TFBIM, ESR1-UTRN, ETV6-NTRK3, EZR-ROS1, FAM114A2-BRAF, FAM193A-FGFR3, FAT1-NTRK3, FBXL20-NOTCH2, FGFR2-AP1M1, FGFR2-BICC1, FGFR2-CASP7, FGFR2-CCAR2, FGFR2-CCDC186, FGFR2-CCDC6, FGFR2-CTNNA3, FGFR2-EIF4A2, FGFR2-ENPP2, FGFR2-FRK, FGFR2-OFD1, FGFR2-SHTN1, FGFR2-SMN1, FGFR2-TACC2, FGFR2-USP10, FGFR3-AES, FGFR3-AMBRA1, FGFR3-ELAVL3, FGFR3-FBX028, FGFR3-MLLT10, FGFR3-TACC3, FKBP15-RET, FOX01-PDGFRB, FRMD3-BRD4, GPRC5A-NRG1, GTF2IRD1-ALK, HDLBP-TMPRSS2, HIBADH-BRAF, HMGN2P46-TMPRSS2, IGHGP-NOTCH1, IRF2BP2-NTRK1, JAK2-CSTF3, JAK2-DOCK8, JAK2-GLDC, JAK2-RCL1, KANSL1L-ERBB4, KCNQ5-ALK, KDM7A-BRAF, KIAA1211-PDGFRA, KIF5B-MET, KLHL7-BRAF, LMNA-NTRK1, LMNA-RAF1, LYN-NTRK3, MACF1-BRAF, MAGI3-NOTCH2, MALATI-ALK, MAP3K7-PDGFRB, MAPK1-NOTCH1, MESDC2-TMPRSS2, MET-C8orf34, MET-CNTNAP5, MET-DYNC1I1, MET-ST7-AS2, MET-TFG, MET-WNT2, MGP-ESR1, MKRN1-BRAF, MPRIP-RAF1, NCOA4-RET, NDUFS4-TMPRSS2, NOTCH1-CHST9, NOTCH1-EXD3, NOTCH1-LCN15, NOTCH1-MAPK1, NOTCH1-SDCCAG3, NOTCH1-SPTAN1, NOTCH1-TMEM117, NOTCH2-ADAM30, NOTCH2-CWH43, NOTCH2-MNDA, NOTCH2-PSMA5, NOTCH2-REG4, NOTCH2-SEC22B, NOTCH2-SPAG17, NRG1-PMEPA1, NRG1-STMN2, NTRK1-DYNC2H1, NTRK3-ETV6, NTRK3-LOXL2, NTRK3-PEAK1, NTRK3-RBPMS, NUP214-ABL1, OXR1-MET, PAICS-PDGFRA, PAPD7-RAF1, PCM1-NRG1, PDE7A-NRG1, PDE9A-TMPRSS2, PDGFRA-FIP1L1, PDGFRA-GRID2, PDGFRA-SCFD2, PDGFRA-USP8, PKHD1-ESR1, PLGRKT-JAK2, PML-RARA, PPP4R3B-ALK, PTGFRN-NOTCH2, PTPRZ1-MET, RAB3IL1-NRG1, RAB5B-ALK, RAC1P2-EGFR, RAF1-AGGF1, RAF1-C9orf153, RAF1-EIF3L, RAF1-GXYLT2, RAF1-IQSEC1, RAF1-NXPH1, RAF1-PHC3, RAF1-RPL32, RAF1-SSUH2, RAF1-TRAK1, RBPMS-NTRK3, RET-CCDC6, RET-MRLN, RET-NCOA4, RHBDD2-EGFR, ROS1-CD74, ROS1-CLTC, ROS1-FBX09, SCP2-TMPRSS2, SDC4-NRG1, SEC61G-EGFR, SIK3-TMPRSS2, SLC34A2-ROS1, SLC45A3-TMPRSS2, SMAD4-NRG1, SMARCA4-BRD4, SMN1-FGFR2, SND1-BRAF, SPECC1L-RET, SQSTM1-NTRK1, SSBP2-NTRK1, STRN-ALK, SYNE1-ESR1, TACC3-FGFR3, TAX1BP1-BRAF, TBL1XR1-RET, TCEA1-EGFR, TFG-MET, TFG-NTRK1, THAP7-NRG1, THBS1-NRG1, TMEFF2-TMPRSS2, TMEM165-PDGFRA, TMPRSS2-ATF7IP, TMPRSS2-BRAF, TMPRSS2-CALB1, TMPRSS2-DGKG, TMPRSS2-DIAPH1, TMPRSS2-EML4, TMPRSS2-ERG, TMPRSS2-ETV4, TMPRSS2-ETV5, TMPRSS2-GUCA2A, TMPRSS2-HDLBP, TMPRSS2-HSF2BP, TMPRSS2-INPP4B, TMPRSS2-IRS2, TMPRSS2-KLF4, TMPRSS2-MORC3, TMPRSS2-RPS6, TMPRSS2-MX1, TMPRSS2-PDE9A, TMPRSS2-PHF12, TMPRSS2-SARS, TMPRSS2-TMEFF2, TMPRSS2-TMEM109, TPM1-ALK, TPM3-NTRK1, TRAK1-RAF1, TRIM24-BRAF, TRIM27-RET, TTC13-JAK2, TULP4-ESR1, UBXN8-NRG1, USP28-TMPRSS2, USP46-PDGFRA, VCL-FGFR2, VPS18-NTRK3, WRN-NRG1, ZBTB7B-NTRK1, ZC3HAVI-BRAF, ZEB2-AKT3, and ZNF430-BRD4.

Example targetable fusions include ALK fusions, such as TFG-ALK. ALK fusions have been identified in multiple cancer types, for example lung adenocarcinoma, bladder, colorectal, breast, renal cell, renal medullary and thyroid cancers. In particular, EML4-ALK fusions were found in lung adenocarcinoma, STRN-ALK fusion in thyroid cancer and in papillary renal carcinoma, TPM1-ALK fusion in bladder cancer, SMEK2-ALK fusion in rectal adenocarcinoma and GTF2IRD1-ALK fusion in thyroid cancer. Another targetable fusion includes BRAF fusions, which are associated with prostate cancer, melanoma, radiation-induced thyroid cancer, and pediatric low-grade gliomas. In particular, TRIM-BRAF fusion has been found in rectal adenocarcinoma, ATG7-BRAF in melanoma, and ZC3HAVI-BRAF as well as FAM114A2-BRAF in thyroid cancer. Other example fusions include AGK-BRAF, SND1-BRAF, MACF1-BRAF, TAX1BP1-BRAF and CDC27-BRAF. It is known in the art BRAF dimers are not sensitive to RAF inhibitors and instead be treated to inhibition downstream through, for example, MEK inhibition.

Another targetable fusion includes FGFR fusions, which have been identified in glioblastoma multiforme, bladder urothelial carcinoma, lung squamous cell carcinoma, kidney papillary cell carcinoma, brain low-grade glioma, prostate adenocarcinoma, head and neck squamous cell carcinoma, breast invasive carcinoma, stomach adenocarcinoma tumor types. In particular, FGFR3-TACC3 fusion has been found in papillary renal carcinoma, FGFR3-ELAVL3 in low-grade glioma and FGFR3-BAIAP2L1 in bladder cancer. Another targetable fusion is WASF2-FGR fusions, which have been found in in lung squamous carcinoma, ovarian serous cystadenocarcinoma and skin cutaneous melanoma. Another targetable fusion includes MET fusions, which have been found in low-grade glioma, hepatocellular carcinoma, lung adenocarcinoma and thyroid carcinoma. In particular, BAIAP2L1-MET and C8orf34-MET have been found in papillary renal carcinoma, KIF5B-MET in lung adenocarcinoma, and TFG-MET in thyroid papillary carcinoma. Another notable fusion is TPR-MET.

Another targetable fusion includes NTRK fusions, which have been associated with congenital fibrosarcoma, human secretory breast carcinoma, and papillary thyroid cancer, including glioblastoma, cholangiocarcinoma and pediatric high-grade glioma. In particular, PAN3-NTRK2 have been found in head and neck squamous cell carcinoma, AFAP1-NTRK2 low-grade glioma, TRIM24-NTRK2 in lung adenocarcinoma, and TPM3-NTRK1 in sarcoma and thyroid cancer. Another targetable fusion includes PIK3CA fusions, which have been found in endometrial cancers, breast invasive carcinomas, and colorectal, head, and neck cancers. In particular, TBL1XR1-PIK3CA fusions have been found in breast cancer and prostate adenocarcinoma, FNDC3B-PIK3CA fusion in uterine corpus endometrial carcinoma, and TBL1XR1-PIK3CA fusions in invasive breast carcinoma and prostate cancer. Another targetable fusion is PKC fusions, which have been found in papillary glioneuronal tumors and benign fibrous histiocytoma. PRKCA fusions have been found in lung squamous cell carcinoma and PRKCB fusions have been found in lung squamous cell carcinoma, lung adenocarcinoma and low-grade glioma. Example fusions include PRKCA was fused with IGF2BP3. TANC2-PRKCA.

Another targetable fusion includes PKN1 fusions and have been found in squamous cell carcinoma of the lung and hepatocellular carcinoma. Example PKN1 fusions include ANXA4-PKN1 and TECR-PKN1. Another targetable fusion includes RAF1, also known as CRAF, fusions, which have been found in melanoma and prostate adenocarcinoma. In particular AGGF1-RAF1 has been found in papillary thyroid carcinoma and prostate cancer. Another targetable fusion includes RET fusions, which have been found in lung adenocarcinoma and thyroid cancer. In particular, CCDC6-RET fusions have been found in thyroid cancer and colon adenocarcinoma while ERC1-RET fusion has been found in breast cancer. Other example fusions include RET with AKAP13, FKBP15, SPECC1L, and TBL1XR1. Another targetable fusion is ROS1 fusions, such as CEP85L-ROS1 which has been found in glioblastoma and single angiosarcoma. Another notable ROS1 fusion is CD74-ROS1 while other fusions have been found in 8/513 lung adenocarcinomas.

Tyrosine kinase fusion genes are a notable class of oncogenes. Tyrosine kinase fusions have been found in leukemia and solid tumors. Like other fusions, they are created by translocations and other chromosomal rearrangements of a subset of tyrosine kinase genes. These fusions include ABL, PDGFRA, PDGFRB, FGFR1, SYK, RET, JAK2 and ALK. The kinase domain is activated by enforced oligomerization and inactivation of inhibitory domains. Activated tyrosine kinase fusions then signal via an array of transduction cascades. The fusion partner recruits proteins that contribute to signaling, protein stability, cellular localization and oligomerization.

See, e.g. Stransky, N., Cerami, E., Schalm, S. et al. The landscape of kinase fusions in cancer. Nat Commun 5, 4846 (2014). doi: 10.1038/ncomms5846 (including, in particular, FIG. 1, providing a landscape of recurrent kinase fusions in solid tumors, incorporated by reference); Medves et al., J Cell Mol Med. 2012 February; 16(2):237-48; doi: 10.1111/j.1582-4934.2011.01415.x. (including, in particular, TK fusions and their inhibitor molecules of Table 1, incorporated by reference); and Gao, Qingsong et al. “Driver Fusions and Their Implications in the Development and Treatment of Human Cancers.” Cell reports vol. 23,1 (2018): 227-238.e3. doi:10.1016/j.celrep.2018.03.050, each of which is incorporated herein by reference in their entirety.

Gao et al. provides a table of potentially druggable fusion events and their targets in Table S5, specifically incorporated herein by reference for its teaching of fusions, targets and indications associated with the fusion events.

Exemplary cancers associated with such fusions include adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, liver hepatocellular carcinoma, lung adenocarcinoma, lung squamous cell carcinoma, lymphoid neoplasm diffuse large B cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, and uveal melanoma.

Isoforms of RAS have conserved amino acid sequences in the Switch-I and Switch-II regions of Ras. The Switch regions of Ras are the binding interface for effector proteins and Ras regulators such as GTPase-activating proteins (GAPs) and guanine nucleotide exchange factors (GEFs). Several cancer mutations are located within Switch II, and the P-loop attached to Switch-I. Thus, phosphorylation of loop residues in the Switch-I or Switch-II may address Ras activity, as post-translation modifications of loop residues are known generally to generate conformational changes.

KRAS is a key regulator of cell proliferation, differentiation and survival, and is the most frequently mutated oncogene in human cancers. An exemplary oncogenic driver mutation is KRAS^{G12C}. The active GTP-bound state of KRAS is a closed conformation, while the inactive, GDP-bound states is an open conformation. In KRAS G12C, and other oncogenic RAS mutations, a dysregulated excess of cellular GTP-bound RAS results, with the RAS function remaining in the open conformation active state that results in uncontrolled cell growth and proliferation, invasiveness and evasion of immune surveillance. Accordingly, inhibition of GTPases (e.g. Ras) is within the scope of the chimeric small molecules disclosed.

Without being bound by a particular scientific theory, it is proposed phosphorylation of KRAS, particularly KRAS^{G12C} may facilitate generation of conformational change, perhaps by disrupting binding to GTPase-activating proteins, thereby decreasing Ras activity which is implicated in oncogenesis. For example, phosphorylation of T35 or S17 residues which coordinate to Mg²⁺ ion that also coordinates to the gamma- and beta-phosphates of GTP can potentially disrupt the 4-way Mg2+ chelation. This tetra-chelated Mg2+ state is characteristic of the active GTP-bound state “closed conformation”) while inactive GDP-bound state only has S17 and the gamma-phosphate of GDP involved in Mg²⁺ binding. Further without being bound by theory, it may be that phosphorylation of any Switch-I or Switch-II or Switch-adjacent residues can disrupt protein-protein interactions between the Switch regions and Ras regulators, and the activating proteins, or that phosphorylation of loop residues in Switch-I or Switch II can cause conformation changes, as post-translational modifications of loop residues are often known to generate conformational changes. Accordingly, modulating KRAS signaling with a kinase utilizing the phosphorylation inducing chimeric small molecules described herein may be useful as an anti-cancer therapy by disrupting KRAS membrane localization or binding partners.

In one aspect, the method comprises treating cancer as a result of KRAS. In one example embodiment, the chimeric small molecule target binding moiety targets KRAS, NF-kB, LDH-A, p53, GP73, MUC1, MUC16, CD44, GPCR, HMGB1, RIOK1, CHK1, UBE2F, HUR, PTEN, STAT-3, Osteopontin, EGFRs, AKT, DAPK1, Rho, Ubc9, FOXK2, HIC1, HER2, BRAF, BCL-2, CD117, (KIT), ALK, PI3K, Delta, DNMT1, SMO.

In one example embodiment, the chimeric small molecule target binding moiety targets MYC, K-RAS, N-RAS, TP53, KDM6A, NPM1, H-RAS, FGFR3, MSH6, TP53, EGFR, PIK3CA, ABLI, CTNNB1, KIT, INFIA, JAK2, BRAF, IDHI, RET, PDGFRA, MET, APC, CDC27, CDK4, prostate-specific antigen, alpha fetoprotein, breast mucin, gplOO, g250, p53, MART-I, MAGE, BAGE, GAGE, tyrosinase, Tyrosinase related protein 11, Tyrosinase related protein, or RAD50.

Additional cancer targets, indications and small molecules target binding moietys are provided in Table 2 of Sharma B S (2019). Post-Translational Modifications (PTMs), from a Cancer Perspective: An Overview. Oncogen 2(3): 12, specifically incorporated herein by reference.

### Diseases/Disorders

In some embodiments, the disease is associated with aberrant protein expression, or expression of a tumor antigen, e.g., a proliferative disease, a precancerous condition, a cancer, or a non-cancer related indication associated with expression of the tumor antigen, which may in some embodiments comprise a target selected from B2M, CD247, CD3D, CD3E, CD3G, TRAC, TRBC1, TRBC2, HLA-A, HLA-B, HLA-C, DCK, CD52, FKBP1A, CIITA, NLRC5, RFXANK, RFX5, RFXAP, or NR3C1, HAVCR2, LAG3, PDCD1, PD-L2, CTLA4, CEACAM (CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, or PTPN11 DCK, CD52, NR3C1, LILRB1, CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); n kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1), CD19, BCMA, CD70, G6PC, Dystrophin, including modification of exon 51 by deletion or excision, DMPK, CFTR (cystic fibrosis transmembrane conductance regulator). In one example embodiment, the targets comprise CD70, or a Knock-in of CD33 and Knock-out of B2M. In one example embodiment, the targets comprise a knockout of TRAC and B2M, or TRAC B2M and PD1, with or without additional target genes. In one example embodiment, the disease is cystic fibrosis with targeting of the SCNN1A gene. In one example embodiment, the modification via the chimeric small molecules is used in multiple sclerosis, e.g. αB-crystallin, or in SLE with multiple targets (see, e.g. Doyle and Mamula, Curr Opin Immunol. 2012).

### Additional Diseases and Disorders

In one example embodiment, the treatment is for disease/disorder of an organ, including liver disease, eye disease, muscle disease, heart disease, blood disease, brain disease, kidney disease, or may comprise treatment for an autoimmune disease, central nervous system disease, cancer and other proliferative diseases, neurodegenerative disorders, inflammatory disease, metabolic disorder, musculoskeletal disorder and the like.

Particular diseases/disorders include chondroplasia, achromatopsia, acid maltase deficiency, adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefelter syndrome, Krabbes Disease, Langer-Giedion Syndrome, leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, and Wiskott-Aldrich syndrome.

In one example embodiment, the disease is associated with expression of a tumor antigen, e.g., a proliferative disease, a precancerous condition, a cancer, or a non-cancer related indication associated with expression of the tumor antigen, which may in some embodiments comprise a target selected from B2M, CD247, CD3D, CD3E, CD3G, TRAC, TRBC1, TRBC2, HLA-A, HLA-B, HLA-C, DCK, CD52, FKBP1A, CIITA, NLRC5, RFXANK, RFX5, RFXAP, or NR3C1, HAVCR2, LAG3, PDCD1, PD-L2, CTLA4, CEACAM (CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, or PTPN11 DCK, CD52, NR3C1, LILRB1, CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); n kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoHI glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1), CD19, BCMA, CD70, G6PC, Dystrophin, DMPK, CFTR (cystic fibrosis transmembrane conductance regulator).

In one example embodiment, the disease is Metachromatic Leukodystrophy, and the target is Arylsulfatase A, the disease is Wiskott-Aldrich Syndrome and the target is Wiskott-Aldrich Syndrome protein, the disease is Adreno leukodystrophy and the target is ATP-binding cassette DI, the disease is Human Immunodeficiency Virus and the target is receptor type 5-C—C chemokine or CXCR4 gene, the disease is Beta-thalassemia and the target is Hemoglobin beta subunit, the disease is X-linked Severe Combined ID receptor subunit gamma and the target is interelukin-2 receptor subunit gamma, the disease is Multisystemic Lysosomal Storage Disorder cystinosis and the target is cystinosin, the disease is Diamon-Blackfan anemia and the target is Ribosomal protein S19, the disease is Fanconi Anemia and the target is Fanconi anemia complementation groups (e.g. FNACA, FNACB, FANCC, FANCD1, FANCD2, FANCE, FANCF, RAD51C), the disease is Shwachman-Bodian-Diamond Bodian-Diamond syndrome and the target is Shwachman syndrome gene, the disease is Gaucher's disease and the target is Glucocerebrosidase, the disease is Hemophilia A and the target is Anti-hemophiliac factor OR Factor VIII, Christmas factor, Serine protease, Factor Hemophilia B IX, the disease is Adenosine deaminase deficiency (ADA-SCID) and the target is Adenosine deaminase, the disease is GM1 gangliosidoses and the target is beta-galactosidase, the disease is Glycogen storage disease type II, Pompe disease, the disease is acid maltase deficiency acid and the target is alpha-glucosidase, the disease is Niemann-Pick disease, SMPDl-associated (Types Sphingomyelin phosphodiesterase 1 OR A and B) acid and the target is sphingomyelinase, the disease is Krabbe disease, globoid cell leukodystrophy and the target is Galactosylceramidase or galactosylceramide lipidosis and the target is galactercerebrosidease, Human leukocyte antigens DR-15, DQ-6, the disease is Multiple Sclerosis (MS) DRB1, the disease is Herpes Simplex Virus 1 or 2. The disease can be Hepatitis B with a target of one or more of PreC, C, X, PreS1, PreS2, S, P and/or SP gene(s).

In one example embodiment, the immune disease is severe combined immunodeficiency (SCID), Omenn syndrome, and in one aspect the target is Recombination Activating Gene 1 (RAG1) or an interleukin-7 receptor (IL7R). In one example embodiment, the disease is Transthyretin Amyloidosis (ATTR), Familial amyloid cardiomyopathy, and in one aspect, the target is the TTR gene, including one or more mutations in the TTR gene. In one example embodiment, the disease is Alpha-1 Antitrypsin Deficiency (AATD) or another disease in which Alpha-1 Antitrypsin is implicated, for example GvHD, Organ transplant rejection, diabetes, liver disease, COPD, Emphysema and Cystic Fibrosis, in one example embodiment, the target is SERPINA1.

In one example embodiment, the disease is primary hyperoxaluria, which, in one example embodiment, the target comprises one or more of Lactate dehydrogenase A (LDHA) and hydroxy Acid Oxidase 1 (HAO 1). In one example embodiment, the disease is primary hyperoxaluria type 1 (ph1) and other alanine-glyoxylate aminotransferase (agxt) gene related conditions or disorders, such as Adenocarcinoma, Chronic Alcoholic Intoxication, Alzheimer's Disease, Cooley's anemia, Aneurysm, Anxiety Disorders, Asthma, Malignant neoplasm of breast, Malignant neoplasm of skin, Renal Cell Carcinoma, Cardiovascular Diseases, Malignant tumor of cervix, Coronary Arteriosclerosis, Coronary heart disease, Diabetes, Diabetes Mellitus, Diabetes Mellitus Non-Insulin-Dependent, Diabetic Nephropathy, Eclampsia, Eczema, Subacute Bacterial Endocarditis, Glioblastoma, Glycogen storage disease type II, Sensorineural Hearing Loss (disorder), Hepatitis, Hepatitis A, Hepatitis B, Homocystinuria, Hereditary Sensory Autonomic Neuropathy Type 1, Hyperaldosteronism, Hypercholesterolemia, Hyperoxaluria, Primary Hyperoxaluria, Hypertensive disease, Inflammatory Bowel Diseases, Kidney Calculi, Kidney Diseases, Chronic Kidney Failure, leiomyosarcoma, Metabolic Diseases, Inborn Errors of Metabolism, Mitral Valve Prolapse Syndrome, Myocardial Infarction, Neoplasm Metastasis, Nephrotic Syndrome, Obesity, Ovarian Diseases, Periodontitis, Polycystic Ovary Syndrome, Kidney Failure, Adult Respiratory Distress Syndrome, Retinal Diseases, Cerebrovascular accident, Turner Syndrome, Viral hepatitis, Tooth Loss, Premature Ovarian Failure, Essential Hypertension, Left Ventricular Hypertrophy, Migraine Disorders, Cutaneous Melanoma, Hypertensive heart disease, Chronic glomerulonephritis, Migraine with Aura, Secondary hypertension, Acute myocardial infarction, Atherosclerosis of aorta, Allergic asthma, pineoblastoma, Malignant neoplasm of lung, Primary hyperoxaluria type I, Primary hyperoxaluria type 2, Inflammatory Breast Carcinoma, Cervix carcinoma, Restenosis, Bleeding ulcer, Generalized glycogen storage disease of infants, Nephrolithiasis, Chronic rejection of renal transplant, Urolithiasis, pricking of skin, Metabolic Syndrome X, Maternal hypertension, Carotid Atherosclerosis, Carcinogenesis, Breast Carcinoma, Carcinoma of lung, Nephronophthisis, Microalbuminuria, Familial Retinoblastoma, Systolic Heart Failure Ischemic stroke, Left ventricular systolic dysfunction, Cauda Equina Paraganglioma, Hepatocarcinogenesis, Chronic Kidney Diseases, Glioblastoma Multiforme, Non-Neoplastic Disorder, Calcium Oxalate Nephrolithiasis, Ablepharon-Macrostomia Syndrome, Coronary Artery Disease, Liver carcinoma, Chronic kidney disease stage 5, Allergic rhinitis (disorder), Crigler Najjar syndrome type 2, and Ischemic Cerebrovascular Accident. In one example embodiment, treatment is targeted to the liver. In one example embodiment, the gene is AGXT, with a cytogenetic location of 2q37.3 and the genomic coordinate are on Chromosome 2 on the forward strand at position 240,868,479-240,880,502.

Treatment can also target collagen type vii alpha 1 chain (col7a1) gene related conditions or disorders, such as Malignant neoplasm of skin, Squamous cell carcinoma, Colorectal Neoplasms, Crohn Disease, Epidermolysis Bullosa, Indirect Inguinal Hernia, Pruritus, Schizophrenia, Dermatologic disorders, Genetic Skin Diseases, Teratoma, Cockayne-Touraine Disease, Epidermolysis Bullosa Acquisita, Epidermolysis Bullosa Dystrophica, Junctional Epidermolysis Bullosa, Hallopeau-Siemens Disease, Bullous Skin Diseases, Agenesis of corpus callosum, Dystrophia unguium, Vesicular Stomatitis, Epidermolysis Bullosa With Congenital Localized Absence Of Skin And Deformity Of Nails, Juvenile Myoclonic Epilepsy, Squamous cell carcinoma of esophagus, Poikiloderma of Kindler, pretibial Epidermolysis bullosa, Dominant dystrophic epidermolysis bullosa albopapular type (disorder), Localized recessive dystrophic epidermolysis bullosa, Generalized dystrophic epidermolysis bullosa, Squamous cell carcinoma of skin, Epidermolysis Bullosa Pruriginosa, Mammary Neoplasms, Epidermolysis Bullosa Simplex Superficialis, Isolated Toenail Dystrophy, Transient bullous dermolysis of the newborn, Autosomal Recessive Epidermolysis Bullosa Dystrophica Localisata Variant, and Autosomal Recessive Epidermolysis Bullosa Dystrophica Inversa.

In one example embodiment, the disease is acute myeloid leukemia (AML), targeting Wilms Tumor I (WTI) and HLA expressing cells. In one example embodiment, the therapy is T cell therapy, as described elsewhere herein, comprising engineered T cells with WTI specific TCRs. In one example embodiment, the target is CD157 in AML.

In one example embodiment, the disease is a blood disease. In one example embodiment, the disease is hemophilia, in one aspect the target is Factor XI. In other embodiments, the disease is a hemoglobinopathy, such as sickle cell disease, sickle cell trait, hemoglobin C disease, hemoglobin C trait, hemoglobin S/C disease, hemoglobin D disease, hemoglobin E disease, a thalassemia, a condition associated with hemoglobin with increased oxygen affinity, a condition associated with hemoglobin with decreased oxygen affinity, unstable hemoglobin disease, methemoglobinemia. Hemostasis and Factor X and XII deficiencies can also be treated. In one example embodiment, the target is BCL11A gene (e.g., a human BCL11a gene), a BCL11a enhancer (e.g., a human BCL11a enhancer), or a HFPH region (e.g., a human HPFH region), beta globulin, fetal hemoglobin, γ-globin genes (e.g., HBG1, HBG2, or HBG1 and HBG2), the erythroid specific enhancer of the BCL11A gene (BCL11Ae), or a combination thereof.

In one example embodiment, the target locus can be one or more of RAC, TRBCl, TRBC2, CD3E, CD3G, CD3D, B2M, CIITA, CD247, HLA-A, HLA-B, HLA-C, DCK, CD52, FKBP1A, NLRC5, RFXANK, RFX5, RFXAP, NR3C1, CD274, HAVCR2, LAG3, PDCD1, PD-L2, HCF2, PAI, TFPI, PLAT, PLAU, PLG, RPOZ, F7, F8, F9, F2, F5, F7, F10, F11, F12, F13A1, F13B, STAT1, FOXP3, IL2RG, DCLREIC, ICOS, MHC2TA, GALNS, HGSNAT, ARSB, RFXAP, CD20, CD81, TNFRSF13B, SEC23B, PKLR, IFNG, SPTB, SPTA, SLC4A1, EPO, EPB42, CSF2 CSF3, VFW, SERPINCA1, CTLA4, CEACAM (e.g., CEACAM-1, CEACAM-3 and/or CEACAM-5), VISTA, BTLA, TIGIT, LAIR1, CD160, 2B4, CD80, CD86, B7-H3 (CD113), B7-H4 (VTCNl), HVEM (TNFRSF14 or CD107), KIR, A2aR, MHC class I, MHC class II, GAL9, adenosine, and TGF beta, PTPN11, and combinations thereof. In one example embodiment, the target sequence within the genomic nucleic acid sequence at Chrl 1:5,250,094-5,250,237,-strand, hg38; Chrl 1:5,255,022-5,255,164,-strand, hg38; nondeletional HFPH region; Chrl 1:5,249,833 to Chrl 1:5,250,237,-strand, hg38; Chrl 1:5,254,738 to Chrl 1:5,255, 164,-strand, hg38; Chrl 1:5,249,833-5,249,927,-strand, hg3; Chrl 1:5,254,738-5,254,851,-strand, hg38; Chrl 1:5,250, 139-5,250,237,-strand, hg38.

In one example embodiment, the disease is associated with high cholesterol, and regulation of cholesterol is provided, in some embodiments, regulation is effected by modification in the target PCSK9. Other diseases in which PCSK9 can be implicated, and thus would be a target for the systems and methods described herein include Abetaiipoproteinemia, Adenoma, Arteriosclerosis, Atherosclerosis, Cardiovascular Diseases, Cholelithiasis, Coronary Arteriosclerosis, Coronary heart disease, Non-Insulin-Dependent Diabetes Meliitus, Hypercholesterolemia, Familial Hypercholesterolemia, Hyperinsuiinism, Hyperlipidemia, Familial Combined Hyperlipidemia, Hypobetalipoproteinemias, Chronic Kidney Failure, Liver diseases, Liver neoplasms, melanoma, Myocardial Infarction, Narcolepsy, Neoplasm Metastasis, Nephroblastoma, Obesity, Peritonitis, Pseudoxanthoma Elasticum, Cerebrovascular accident, Vascular Diseases, Xanthomatosis, Peripheral Vascular Diseases, Myocardial Ischemia, Dyslipidemias, Impaired glucose tolerance, Xanthoma, Polygenic hypercholesterolemia, Secondary malignant neoplasm of liver, Dementia, Overweight, Hepatitis C, Chronic, Carotid Atherosclerosis, Hyperlipoproteinemia Type Ha, Intracranial Atherosclerosis, Ischemic stroke, Acute Coronary Syndrome, Aortic calcification, Cardiovascular morbidity, Hyperlipoproteinemia Type lib, Peripheral Arterial Diseases, Familial Hyperaldosteronism Type II, Familial hypobetalipoproteinemia, Autosomal Recessive Hypercholesterolemia, Autosomal Dominant Hypercholesterolemia 3, Coronary Artery Disease, Liver carcinoma, Ischemic Cerebrovascular Accident, and Arteriosclerotic cardiovascular disease NOS. In one example embodiment, the treatment can be targeted to the liver, the primary location of activity of PCSK9.

In one example embodiment, the disease or disorder is Hyper IGM syndrome or a disorder characterized by defective CD40 signaling. In one example embodiment, the insertion of CD40L exons are used to restore proper CD40 signaling and B cell class switch recombination. In one example embodiment, the target is CD40 ligand (CD40L)-edited at one or more of exons 2-5 of the CD40L gene, in cells, e.g., T cells or hematopoietic stem cells (HSCs).

In one example embodiment, the disease is merosin-deficient congenital muscular dystrophy (mdcmd) and other laminin, alpha 2 (lama2) gene related conditions or disorders. The therapy can be targeted to the muscle, for example, skeletal muscle, smooth muscle, and/or cardiac muscle. In one example embodiments, the target is Laminin, Alpha 2 (LAMA2) which may also be referred to as Laminin-12 Subunit Alpha, Laminin-2 Subunit Alpha, Laminin-4 Subunit Alpha 3, Merosin Heavy Chain, Laminin M Chain, LAMM, Congenital Muscular Dystrophy and Merosin. LAMA2 has a cytogenetic location of 6q22.33 and the genomic coordinate are on Chromosome 6 on the forward strand at position 128,883, 141-129,516,563. In one example embodiment, the disease treated can be Merosin-Deficient Congenital Muscular Dystrophy (MDCMD), Amyotrophic Lateral Sclerosis, Bladder Neoplasm, Charcot-Marie-Tooth Disease, Colorectal Carcinoma, Contracture, Cyst, Duchenne Muscular Dystrophy, Fatigue, Hyperopia, Renovascular Hypertension, melanoma, Mental Retardation, Myopathy, Muscular Dystrophy, Myopia, Myositis, Neuromuscular Diseases, Peripheral Neuropathy, Refractive Errors, Schizophrenia, Severe mental retardation (I.Q. 20-34), Thyroid Neoplasm, Tobacco Use Disorder, Severe Combined Immunodeficiency, Synovial Cyst, Adenocarcinoma of lung (disorder), Tumor Progression, Strawberry nevus of skin, Muscle degeneration, Microdontia (disorder), Walker-Warburg congenital muscular dystrophy, Chronic Periodontitis, Leukoencephalopathies, Impaired cognition, Fukuyama Type Congenital Muscular Dystrophy, Scleroatonic muscular dystrophy, Eichsfeld type congenital muscular dystrophy, Neuropathy, Muscle eye brain disease, Limb-Muscular Dystrophies, Girdle, Congenital muscular dystrophy (disorder), Muscle fibrosis, cancer recurrence, Drug Resistant Epilepsy, Respiratory Failure, Myxoid cyst, Abnormal breathing, Muscular dystrophy congenital merosin negative, Colorectal Cancer, Congenital Muscular Dystrophy due to Partial LAMA2 Deficiency, and Autosomal Dominant Craniometaphyseal Dysplasia.

In one aspect, the target is superoxide dismutase 1, soluble (SOD1), which can aid in treatment of a disease or disorder associated with the gene. In one example embodiment, the disease or disorder is associated with SOD1, and can be, for example, Adenocarcinoma, Albuminuria, Chronic Alcoholic Intoxication, Alzheimer's Disease, Amnesia, Amyloidosis, Amyotrophic Lateral Sclerosis, Anemia, Autoimmune hemolytic anemia, Sickle Cell Anemia, Anoxia, Anxiety Disorders, Aortic Diseases, Arteriosclerosis, Rheumatoid Arthritis, Asphyxia Neonatorum, Asthma, Atherosclerosis, Autistic Disorder, Autoimmune Diseases, Barrett Esophagus, Behcet Syndrome, Malignant neoplasm of urinary bladder, Brain Neoplasms, Malignant neoplasm of breast, Oral candidiasis, Malignant tumor of colon, Bronchogenic Carcinoma, Non-Small Cell Lung Carcinoma, Squamous cell carcinoma, Transitional Cell Carcinoma, Cardiovascular Diseases, Carotid Artery Thrombosis, Neoplastic Cell Transformation, Cerebral Infarction, Brain Ischemia, Transient Ischemic Attack, Charcot-Marie-Tooth Disease, Cholera, Colitis, Colorectal Carcinoma, Coronary Arteriosclerosis, Coronary heart disease, Infection by Cryptococcus neoformans, Deafness, Cessation of life, Deglutition Disorders, Presenile dementia, Depressive disorder, Contact Dermatitis, Diabetes, Diabetes Mellitus, Experimental Diabetes Mellitus, Insulin-Dependent Diabetes Mellitus, Non-Insulin-Dependent Diabetes Mellitus, Diabetic Angiopathies, Diabetic Nephropathy, Diabetic Retinopathy, Down Syndrome, Dwarfism, Edema, Japanese Encephalitis, Toxic Epidermal Necrolysis, Temporal Lobe Epilepsy, Exanthema, Muscular fasciculation, Alcoholic Fatty Liver, Fetal Growth Retardation, Fibromyalgia, Fibrosarcoma, Fragile X Syndrome, Giardiasis, Glioblastoma, Glioma, Headache, Partial Hearing Loss, Cardiac Arrest, Heart failure, Atrial Septal Defects, Helminthiasis, Hemochromatosis, Hemolysis (disorder), Chronic Hepatitis, HIV Infections, Huntington Disease, Hypercholesterolemia, Hyperglycemia, Hyperplasia, Hypertensive disease, Hyperthyroidism, Hypopituitarism, Hypoproteinemia, Hypotension, natural Hypothermia, Hypothyroidism, Immunologic Deficiency Syndromes, Immune System Diseases, Inflammation, Inflammatory Bowel Diseases, Influenza, Intestinal Diseases, Ischemia, Kearns-Sayre syndrome, Keratoconus, Kidney Calculi, Kidney Diseases, Acute Kidney Failure, Chronic Kidney Failure, Polycystic Kidney Diseases, leukemia, Myeloid Leukemia, Acute Promyelocytic Leukemia, Liver Cirrhosis, Liver diseases, Liver neoplasms, Locked-In Syndrome, Chronic Obstructive Airway Disease, Lung Neoplasms, Systemic Lupus Erythematosus, Non-Hodgkin Lymphoma, Machado-Joseph Disease, Malaria, Malignant neoplasm of stomach, Animal Mammary Neoplasms, Marfan Syndrome, Meningomyelocele, Mental Retardation, Mitral Valve Stenosis, Acquired Dental Fluorosis, Movement Disorders, Multiple Sclerosis, Muscle Rigidity, Muscle Spasticity, Muscular Atrophy, Spinal Muscular Atrophy, Myopathy, Mycoses, Myocardial Infarction, Myocardial Reperfusion Injury, Necrosis, Nephrosis, Nephrotic Syndrome, Nerve Degeneration, nervous system disorder, Neuralgia, Neuroblastoma, Neuroma, Neuromuscular Diseases, Obesity, Occupational Diseases, Ocular Hypertension, Oligospermia, Degenerative polyarthritis, Osteoporosis, Ovarian Carcinoma, Pain, Pancreatitis, Papillon-Lefevre Disease, Paresis, Parkinson Disease, Phenylketonurias, Pituitary Diseases, Pre-Eclampsia, Prostatic Neoplasms, Protein Deficiency, Proteinuria, Psoriasis, Pulmonary Fibrosis, Renal Artery Obstruction, Reperfusion Injury, Retinal Degeneration, Retinal Diseases, Retinoblastoma, Schistosomiasis, Schistosomiasis mansoni, Schizophrenia, Scrapie, Seizures, Age-related cataract, Compression of spinal cord, Cerebrovascular accident, Subarachnoid Hemorrhage, Progressive supranuclear palsy, Tetanus, Trisomy, Turner Syndrome, Unipolar Depression, Urticaria, Vitiligo, Vocal Cord Paralysis, Intestinal Volvulus, Weight Gain, HMN (Hereditary Motor Neuropathy) Proximal Type I, Holoprosencephaly, Motor Neuron Disease, Neurofibrillary degeneration (morphologic abnormality), Burning sensation, Apathy, Mood swings, Synovial Cyst, Cataract, Migraine Disorders, Sciatic Neuropathy, Sensory neuropathy, Atrophic condition of skin, Muscle Weakness, Esophageal carcinoma, Lingual-Facial-Buccal Dyskinesia, Idiopathic pulmonary hypertension, Lateral Sclerosis, Migraine with Aura, Mixed Conductive-Sensorineural Hearing Loss, Iron deficiency anemia, Malnutrition, Prion Diseases, Mitochondrial Myopathies, MELAS Syndrome, Chronic progressive external ophthalmoplegia, General Paralysis, Premature aging syndrome, Fibrillation, Psychiatric symptom, Memory impairment, Muscle degeneration, Neurologic Symptoms, Gastric hemorrhage, Pancreatic carcinoma, Pick Disease of the Brain, Liver Fibrosis, Malignant neoplasm of lung, Age related macular degeneration, Parkinsonian Disorders, Disease Progression, Hypocupremia, Cytochrome-c Oxidase Deficiency, Essential Tremor, Familial Motor Neuron Disease, Lower Motor Neuron Disease, Degenerative myelopathy, Diabetic Polyneuropathies, Liver and Intrahepatic Biliary Tract Carcinoma, Persian Gulf Syndrome, Senile Plaques, Atrophic, Frontotemporal dementia, Semantic Dementia, Common Migraine, Impaired cognition, Malignant neoplasm of liver, Malignant neoplasm of pancreas, Malignant neoplasm of prostate, Pure Autonomic Failure, Motor symptoms, Spastic, Dementia, Neurodegenerative Disorders, Chronic Hepatitis C, Guam Form Amyotrophic Lateral Sclerosis, Stiff limbs, Multisystem disorder, Loss of scalp hair, Prostate carcinoma, Hepatopulmonary Syndrome, Hashimoto Disease, Progressive Neoplastic Disease, Breast Carcinoma, Terminal illness, Carcinoma of lung, Tardive Dyskinesia, Secondary malignant neoplasm of lymph node, Colon Carcinoma, Stomach Carcinoma, Central neuroblastoma, Dissecting aneurysm of the thoracic aorta, Diabetic macular edema, Microalbuminuria, Middle Cerebral Artery Occlusion, Middle Cerebral Artery Infarction, Upper motor neuron signs, Frontotemporal Lobar Degeneration, Memory Loss, Classical phenylketonuria, CADASIL Syndrome, Neurologic Gait Disorders, Spinocerebellar Ataxia Type 2, Spinal Cord Ischemia, Lewy Body Disease, Muscular Atrophy, Spinobulbar, Chromosome 21 monosomy, Thrombocytosis, Spots on skin, Drug-Induced Liver Injury, Hereditary Leber Optic Atrophy, Cerebral Ischemia, ovarian neoplasm, Tauopathies, Macroangiopathy, Persistent pulmonary hypertension, Malignant neoplasm of ovary, Myxoid cyst, Drusen, Sarcoma, Weight decreased, Major Depressive Disorder, Mild cognitive disorder, Degenerative disorder, Partial Trisomy, Cardiovascular morbidity, hearing impairment, Cognitive changes, Ureteral Calculi, Mammary Neoplasms, Colorectal Cancer, Chronic Kidney Diseases, Minimal Change Nephrotic Syndrome, Non-Neoplastic Disorder, X-Linked Bulbo-Spinal Atrophy, Mammographic Density, Normal Tension Glaucoma Susceptibility To Finding), Vitiligo-Associated Multiple Autoimmune Disease Susceptibility 1 (Finding), Amyotrophic Lateral Sclerosis And/Or Frontotemporal Dementia 1, Amyotrophic Lateral Sclerosis 1, Sporadic Amyotrophic Lateral Sclerosis, monomelic Amyotrophy, Coronary Artery Disease, Transformed migraine, Regurgitation, Urothelial Carcinoma, Motor disturbances, Liver carcinoma, Protein Misfolding Disorders, TDP-43 Proteinopathies, Promyelocytic leukemia, Weight Gain Adverse Event, Mitochondrial cytopathy, Idiopathic pulmonary arterial hypertension, Progressive cGVHD, Infection, GRN-related frontotemporal dementia, Mitochondrial pathology, and Hearing Loss.

In one example embodiment, the disease is associated with the gene ATXN1, ATXN2, or ATXN3, which may be targeted for treatment. In some embodiments, the CAG repeat region located in exon 8 of ATXN1, exon 1 of ATXN2, or exon 10 of the ATXN3 is targeted. In one example embodiment, the disease is spinocerebellar ataxia 3 (sca3), sca1, or sca2 and other related disorders, such as Congenital Abnormality, Alzheimer's Disease, Amyotrophic Lateral Sclerosis, Ataxia, Ataxia Telangiectasia, Cerebellar Ataxia, Cerebellar Diseases, Chorea, Cleft Palate, Cystic Fibrosis, Mental Depression, Depressive disorder, Dystonia, Esophageal Neoplasms, Exotropia, Cardiac Arrest, Huntington Disease, Machado-Joseph Disease, Movement Disorders, Muscular Dystrophy, Myotonic Dystrophy, Narcolepsy, Nerve Degeneration, Neuroblastoma, Parkinson Disease, Peripheral Neuropathy, Restless Legs Syndrome, Retinal Degeneration, Retinitis Pigmentosa, Schizophrenia, Shy-Drager Syndrome, Sleep disturbances, Hereditary Spastic Paraplegia, Thromboembolism, Stiff-Person Syndrome, Spinocerebellar Ataxia, Esophageal carcinoma, Polyneuropathy, Effects of heat, Muscle twitch, Extrapyramidal sign, Ataxic, Neurologic Symptoms, Cerebral atrophy, Parkinsonian Disorders, Protein S Deficiency, Cerebellar degeneration, Familial Amyloid Neuropathy Portuguese Type, Spastic syndrome, Vertical Nystagmus, Nystagmus End-Position, Antithrombin III Deficiency, Atrophic, Complicated hereditary spastic paraplegia, Multiple System Atrophy, Pallidoluysian degeneration, Dystonia Disorders, Pure Autonomic Failure, Thrombophilia, Protein C, Deficiency, Congenital Myotonic Dystrophy, Motor symptoms, Neuropathy, Neurodegenerative Disorders, Malignant neoplasm of esophagus, Visual disturbance, Activated Protein C Resistance, Terminal illness, Myokymia, Central neuroblastoma, Dyssomnias, Appendicular Ataxia, Narcolepsy-Cataplexy Syndrome, Machado-Joseph Disease Type I, Machado-Joseph Disease Type II, Machado-Joseph Disease Type III, Dentatorubral-Pallidoluysian Atrophy, Gait Ataxia, Spinocerebellar Ataxia Type 1, Spinocerebellar Ataxia Type 2, Spinocerebellar Ataxia Type 6 (disorder), Spinocerebellar Ataxia Type 7, Muscular Spinobulbar Atrophy, Genomic Instability, Episodic ataxia type 2 (disorder), Bulbo-Spinal Atrophy X-Linked, Fragile X Tremor/Ataxia Syndrome, Thrombophilia Due to Activated Protein C Resistance (Disorder), Amyotrophic Lateral Sclerosis 1, Neuronal Intranuclear Inclusion Disease, Hereditary Antithrombin Iii Deficiency, and Late-Onset Parkinson Disease.

In one example embodiment, the disease is associated with expression of a tumor antigen-cancer or non-cancer related indication, for example acute lymphoid leukemia, diffuse large B cell lymphoma, follicular lymphoma, chronic lymphocytic leukemia, Hodgkin lymphoma, non-Hodgkin lymphoma. In one example embodiment, the target can be TET2 intron, a TET2 intron-exon junction, a sequence within a genomic region of chr4.

In one example embodiment, neurodegenerative diseases can be treated. In one example embodiment, the target is Synuclein, Alpha (SNCA). In one example embodiment, the disorder treated is a pain related disorder, including congenital pain insensitivity, Compressive Neuropathies, Paroxysmal Extreme Pain Disorder, High grade atrioventricular block, Small Fiber Neuropathy, and Familial Episodic Pain Syndrome 2. In one example embodiment, the target is Sodium Channel, Voltage Gated, Type X Alpha Subunit (SCNIOA).

In one example embodiment, hematopoetic stem cells and progenitor stem cells are modified, including for treatment of lysosomal storage diseases, glycogen storage diseases, mucopolysaccharoidoses, or any disease in which the secretion of a protein will ameliorate the disease. In one embodiment, the disease is sickle cell disease (SCD). In another embodiment, the disease is β-thalessemia.

Methods and systems can target Dystrophia Myotonica-Protein Kinase (DMPK).

Disorders or diseases associated with DMPK include Atherosclerosis, Azoospermia, Hypertrophic Cardiomyopathy, Celiac Disease, Congenital chromosomal disease, Diabetes Mellitus, Focal glomerulosclerosis, Huntington Disease, Hypogonadism, Muscular Atrophy, Myopathy, Muscular Dystrophy, Myotonia, Myotonic Dystrophy, Neuromuscular Diseases, Optic Atrophy, Paresis, Schizophrenia, Cataract, Spinocerebellar Ataxia, Muscle Weakness, Adrenoleukodystrophy, Centronuclear myopathy, Interstitial fibrosis, myotonic muscular dystrophy, Abnormal mental state, X-linked Charcot-Marie-Tooth disease 1, Congenital Myotonic Dystrophy, Bilateral cataracts (disorder), Congenital Fiber Type Disproportion, Myotonic Disorders, Multisystem disorder, 3-Methylglutaconic aciduria type 3, cardiac event, Cardiogenic Syncope, Congenital Structural Myopathy, Mental handicap, Adrenomyeloneuropathy, Dystrophia myotonica 2, and Intellectual Disability.

In one example embodiment, the disease is an inborn error of metabolism. The disease may be selected from Disorders of Carbohydrate Metabolism (glycogen storage disease, G6PD deficiency), Disorders of Amino Acid Metabolism (phenylketonuria, maple syrup urine disease, glutaric acidemia type 1), Urea Cycle Disorder or Urea Cycle Defects (carbamoyl phosphate synthease I deficiency), Disorders of Organic Acid Metabolism (alkaptonuria, 2-hydroxyglutaric acidurias), Disorders of Fatty Acid Oxidation/Mitochondrial Metabolism (Medium-chain acyl-coenzyme A dehydrogenase deficiency), Disorders of Porphyrin metabolism (acute intermittent porphyria), Disorders of Purine/Pyrimidine Metabolism (Lesch-Nynan syndrome), Disorders of Steroid Metabolism (lipoid congenital adrenal hyperplasia, congenital adrenal hyperplasia), Disorders of Mitochondrial Function (Kearns-Sayre syndrome), Disorders of Peroxisomal function (Zellweger syndrome), or Lysosomal Storage Disorders (Gaucher's disease, Niemann-Pick disease).

In one example embodiment, the target can comprise Recombination Activating Gene 1 (RAG1), BCL11 A, PCSK9, laminin, alpha 2 (lama2), ATXN3, alanine-glyoxylate aminotransferase (AGXT), collagen type vii alpha 1 chain (COL7a1), spinocerebellar ataxia type 1 protein (ATXN1), Angiopoietin-like 3 (ANGPTL3), Frataxin (FXN), Superoxidase Dismutase 1, soluble (SOD1), Synuclein, Alpha (SNCA), Sodium Channel, Voltage Gated, Type X Alpha Subunit (SCN10A), Spinocerebellar Ataxia Type 2 Protein (ATXN2), Dystrophia Myotonica-Protein Kinase (DMPK), beta globin locus on chromosome 11, acyl-coenzyme A dehydrogenase for medium chain fatty acids (ACADM), long-chain 3-hydroxyl-coenzyme A dehydrogenase for long chain fatty acids (HADHA), acyl-coenzyme A dehydrogenase for very long-chain fatty acids (ACADVL), Apolipoprotein C3 (APOCIII), Transthyretin (TTR), Angiopoietin-like 4 (ANGPTL4), Sodium Voltage-Gated Channel Alpha Subunit 9 (SCN9A), Interleukin-7 receptor (IL7R), glucose-6-phosphatase, catalytic (G6PC), haemochromatosis (HFE), SERPINA1, C9ORF72, β-globin, dystrophin, γ-globin.

In one example embodiment, the disease or disorder is associated with Apolipoprotein C3 (APOCIII), which can be targeted for editing. In example embodiments, the disease or disorder may be Dyslipidemias, Hyperalphalipoproteinemia Type 2, Lupus Nephritis, Wilms Tumor 5, Morbid obesity and spermatogenic, Glaucoma, Diabetic Retinopathy, Arthrogryposis renal dysfunction cholestasis syndrome, Cognition Disorders, Altered response to myocardial infarction, Glucose Intolerance, Positive regulation of triglyceride biosynthetic process, Renal Insufficiency, Chronic, Hyperlipidemias, Chronic Kidney Failure, Apolipoprotein C-III Deficiency, Coronary Disease, Neonatal Diabetes Mellitus, Neonatal, with Congenital Hypothyroidism, Hypercholesterolemia Autosomal Dominant 3, Hyperlipoproteinemia Type III, Hyperthyroidism, Coronary Artery Disease, Renal Artery Obstruction, Metabolic Syndrome X, Hyperlipidemia, Familial Combined, Insulin Resistance, Transient infantile hypertriglyceridemia, Diabetic Nephropathies, Diabetes Mellitus (Type 1), Nephrotic Syndrome Type 5 with or without ocular abnormalities, and Hemorrhagic Fever with renal syndrome.

In one example embodiment, the target is Angiopoietin-like 4 (ANGPTL4). Diseases or disorders associated with ANGPTL4 that can be treated include ANGPTL4 is associated with dyslipidemias, low plasma triglyceride levels, regulator of angiogenesis and modulate tumorigenesis, and severe diabetic retinopathy, both proliferative diabetic retinopathy and non-proliferative diabetic retinopathy.

In one example embodiment, the target binding moiety binds to the target protein of interest in order to induce phosphorylation from kinases, even if the target protein of interest, i.e. target substrate, is not a substrate for the kinase. One such protein is in the bromodomain family of proteins. Bromodomains are a family of (−110 amino acid) structurally and evolutionary conserved protein interaction modules that specifically recognize acetylated lysines present in substrate proteins, notably histones. Bromodomains exist as components of large multidomain nuclear proteins that are associated with chromatin remodeling, cell signaling and transcriptional control. Examples of bromodomain-containing proteins with known functions include: (i) histone acetyltransferases (HATs), including CREBBP, GCN5, PCAF and TAFII250; (ii) methyltransferases such as ASH1L and MLL; (iii) components of chromatin-remodeling complexes such as Swi2/Snf2; and (iv) a number of transcriptional regulators (Florence et al. Front. Biosci. 2001, 6, D1008-1018, hereby incorporated by reference in its entirety.).

Bromodomain mediated or BET-mediated such as BRD2-mediated, BRD3-mediated, BRD4-mediated, and/or BRDT-mediated disorders or conditions may be any disease or other deleterious condition in which one or more of the bromodomain-containing proteins, such as BET proteins including BRD2, BRD3, BRD4 and/or BRDT, or a mutant thereof, are known to play a role. Accordingly, another embodiment of the present disclosure relates to treating or lessening the severity of one or more diseases in which one or more of the bromodomain-containing proteins, such as BET proteins, such as BRD2, BRD3, BRD4, and/or BRDT, or a mutant thereof, are known to play a role. For example, a disease or condition in which the biological function of bromodomain affects the development and/or course of the disease or condition, and/or in which modulation of bromodomain alters the development, course, and/or symptoms. Bromodomain mediated disease or condition includes a disease or condition for which bromodomain inhibition provides a therapeutic benefit, e.g. wherein treatment with bromodomain inhibitors, including compounds described herein, provides a therapeutic benefit to the subject suffering from or at risk of the disease or condition. Compounds for inhibiting bromodomains or bromodomain inhibitors are typically compounds which inhibit the binding of a bromodomain with its cognate acetylated proteins, for example, the bromodomain inhibitor is a compound which inhibits the binding of a bromodomain to acetylated lysine residues.

Methods for modifying a protein of interest are also provided, the method comprising contacting the protein of interest with a compound disclosed herein in an environment comprising one or more activators. Methods for the treatment of a disease, disorder, or condition in a subject in need thereof can comprise administering a molecule disclosed herein to a subject.

### Pathogenic Applications

The chimeric small molecules disclosed herein can be utilized in methods of treating infection by a pathogen. Methods of treating infection by a pathogen can include generating a repurposed/reprogrammed cellular protein by administering a chimeric small molecule, as described herein. The chimeric small molecule labels the cellular modifying polypeptide with a pathogenic target binding moiety via the electrophilic reactive group moiety. The electrophilic reactive group reacts with and bonds to a nucleophilic side chain on the cellular modifying polypeptide. Labelling of the cellular modifying polypeptide can allow for bonding to, or association with, the pathogenic target protein via the target binding moiety, or to a molecule in proximity to the pathogenic target protein, thereby facilitating modification of the target substrate. In one aspect, the methods comprise inducing phosphorylation of the target substrate, i.e., pathogenic protein, in or on the cell. The methods may comprise contacting the pathogenic protein with the chimeric small molecule. In one example embodiment, the pathogenic protein is in proximity to a kinase specific to the kinase binding moiety of the molecule. Chimeric small molecules that induce phosphorylation can be optionally provided with adenosine monophosphate (AMP) or another molecule providing an additional phosphate group. Without being bound by theory, the addition of the AMP or other phosphate providing molecule can enhance phosphorylation.

Methods for treating infection by a pathogen are provided. The method comprises, generating a reprogrammed cellular protein by administering to a subject in need thereof a chimeric small molecule of the formula: A-L₁-E-B or A-L₁-E-L₂-B, wherein A is an protein binding moiety; L₁ and L₂ is a linker; E is an electrophilic reactive group and B is a pathogen protein to be modified, whereby the chimeric small molecule labels the cellular protein with the target binding moiety for the target substrate; and modifying the pathogen protein by binding of the repurposed/reprogrammed protein to the pathogen protein via the target binding moiety, whereby the repurposed/reprogrammed cellular protein introduces one or more modifications to the target substrate. In one example embodiment, the cellular protein to be reprogrammed is a oxidoreductase, transferase, hydrolase, lyase, isomerase, ligase, translocase. In one example embodiment, the pathogen is a viruses, bacteria, fungi, or protozoa. In one example embodiment, the bacteria is Mycobacterium tuberculosis (Mtb) or Pseudomonas aeruginosa (PsA). In one example embodiment, the pathogen is Mtb and the pathogen protein is one or more of PtpA, PtpB, SapM, ESAT-6, and Rv2966c. In one example embodiment, the pathogen is (PsA) and the target binding moiety is Colistin. In one example embodiment, the modifying polypeptide binding moiety is a modifying polypeptide inhibitor. In one example embodiment, the modifying polypeptide inhibitor is a promiscuous inhibitor. In one example embodiment, a step of administering a coupling molecule thereby quenching the inhibitor activity of the modifying polypeptide inhibitor is provided.

A pathogen may include viruses, bacteria, fungi, and protozoa. In one example embodiment, the pathogen is a pathogenic bacteria and may include: spirochetes; spirilla; vibrios; gram-negative aerobic rods and cocci; enterics; pyogenic cocci; and endospore-forming bacteria; actinomycetes and related bacteria; rickettsias and chlamydiae; mycoplasmas, which are groups defined by some bacteriological criteria. A pathogenic bacteria may include: Escherichia coli, Salmonella enterica, Salmonella typhi, Shigella dysenteriae, Yersina pestis, Pseudomonas aeruginosa, Vibrio cholerae, Bordetella pertussis, Haemophilus influenza, Helicobacter pylori, Campylobacter jejuni, Neisseria gonorrhoeae, Neisseria meningitidis, Brucella abortus, Bacteroides fragilis, Staphylococcus aureus, Streptococcus pyogenes, Streptococcus pneumoniae, Bacillus anthracis, Bacillus cereus, Clostridium tetani, Clostridium perfringens, Clostridium botulinum, Clostridium difficile, Corynebacterium diphtheriae, Listeria monocytogenes, Mycobacterium tuberculosis, Mycobacterium leprae, Chlamydia trachomatis, Chlamydia pneumoniae, Mycoplasma pneumoniae, Rickettisas, Treponema pallidum, Borrelia burgdorferi, or a variant thereof. (Todar, K. Textbook of Bacteriology (2020) Online)

A target virus may belong to any morphological category including helical, envelope, or icosahedral. A target virus may comprise of DNA or RNA, may be single stranded or double stranded, and may be linear or circular. The genome of the virus may be one nucleic acid molecule or several nucleic acid segments. A target virus may belong to the family: Adenoviridae, Papovaviridae, Parvoviridae, Herpesviridae, Poxviridae, Anelloviridae, Pleolipoviridae, Reoviridae, Picornaviridae, Caliciviridae, Togaviridae, Arenaviridae, Flaviviridae, Orthomyxoviridae, Paramyxoviridae, Bunyaviridae, Rhabdoviridae, Filoviridae, Astroviridae, Bornaviridae, Arteriviridae, Hepeviridae, Retroviridae, Caulimoviridae, Hepadnaviridae, Coronaviridae. In one example embodiment, the virus is SARS-COV-2. (Gelderblom H R. Structure and Classification of Viruses. In: Baron S, editor. Medical Microbiology. 4th edition. Galveston (TX): University of Texas Medical Branch at Galveston; 1996. Chapter 41).

In an exemplary embodiment, the pathogen is a pathogenic fungi and may include: Aspergillus; Blastomyces; Candida; Coccidioides; Cryptococcus; Fusarium; Microsporum; Epidermophyton; Trichophyton; Histoplasma; Rhizopus; Mucor; Rhizomucor; Syncephalastrum; Cunninghamella; Apophysomyces; Lichtheimia (formerly Absidia); Eumycetoma; Pneumocystis; Trichophyton; Microsporum; Epidermophyton; Sporothrix; Paracoccidioides; Talaromyces or a variant or species thereof. (CDC)

In an exemplary embodiment, the pathogen is a pathogenic protozoa belonging to the group: Sarcodina; Mastigophora; Ciliophora; or Sporozoa defined by their mode of movement. (CDC) In one example embodiment, the pathogenic protozoa may include: Entamoeba; Trichomonas; Leishmania; Chilomonas; Giardia; Isopora; Sarcocystis; Nosema; Balantidium; Eimeria; Histomonas; Trypanosoma; Plasmodium; Babesia; or Haemoproteus or a variant or species thereof.

In one aspect, the method comprises treating infection by a pathogen as a result of M. tb. In an example embodiment, the pathogenic target is Mycobacterium tuberculosis (M. tb). Successful infection of host macrophages by M. tb hinges on the weakening of the diverse microbicidal responses by the host cell. M. tb furthers infection by impeding the host cellular signaling machinery. Therefore, infection can be inhibited by phosphorylating proteins associated with M. tb, which promotes increased signaling to the immune system. In one example embodiment, the chimeric small molecule target binding moiety targets the pathogen is M.tb and the pathogen protein is one or more of PtpA, PtpB, SapM, ESAT-6, and Rv2966c.

In a preferred embodiment, the target protein is Mtb protein tyrosine phosphatases (Ptp). Ptps belong to a large family of signaling enzymes and are required for optimal bacillary survival. PTPs, with protein tyrosine kinases, regulate numerous cellular functions, such as cell growth, proliferation, differentiation, metabolism, and immune response. M.tb encodes PtpA and PtpB and secretes them into the cytoplasm of host macrophages. PtpA prevents phagolysosome acidification by dephosphorylation of its substrate, Human Vacuolar Protein Sorting 33B. This results in the exclusion of the macrophage vacuolar-H⁺-ATPase (V-ATPase) from the vesicle. Once inside the macrophage, mPTPB activates Akt signaling and simultaneously blocks ERK1/2 and p38 activation thereby preventing host macrophage apoptosis and cytokine production. Inhibition of PtpA and PtpB decreases Mtb survival. See e.g. Dutta N. K. et al. Mycobacterial Protein Tyrosine Phosphatases A and B Inhibitors Augment the Bactericidal Activity of the Standard Anti-tuberculosis Regimen. ACS Infect Dis. 2016; 2(3):231-239 and Ruddraraju, K. V. et al. Therapeutic Targeting of Protein Tyrosine Phosphatases from Mycobacterium tuberculosis. Microorganisms 2021, 9(1), 14 incorporated herein by reference.

In an example embodiment, a method of treating Mycobacterium tuberculosis in macrophages is provided, comprising administering chimeric small molecule of the present invention. In one example the small molecule comprises a MAPK kinase binder, a linker, an electrophilic reactive group, and a Mycobacterium target binding moiety. In one example embodiment, the molecule comprises a binder of MAPK p38a based on the inhibitor SB203580 that further comprises an azide bioorthogonal group. The binding moiety comprises a linker connected to the electrophilic reactive group N-acyl-N-alkyl sulfonamide. The electrophilic reactive group, in turn, is connected to the Mtb protein target binding moiety for PtpA via a linker. Upon binding to the MAPK kinase via SB203580, proximal lysines of the binding pocket of p38a MAPK, K15, K54, K66, K152, K165, will react with the lysine-reactive group (e.g., N-acyl-N-alkyl sulfonamide) and expel the kinase inhibitor, leaving the MAPK tagged with the PtpA binder. The kinase which is covalently labeled with the PtpA binder can then hyper and/or neo-phosphorylate the Mtb PtpA proteins, which can lead to HLA-display and generation of a strong immune response against the pathogen-specific phosphopeptides, allowing deactivation and elimination of infected macrophages by the immune system. Administration of a cyclooctyne coupling molecule can quench the azide biorthogonal group displayed on the kinase binding molecule and deactivate the expelled kinase binding moiety.

In one preferred embodiment, the target protein is SapM. M. tb produces SapM, a alkaline phosphatase, for survival as it inhibits phagosome maturation in host macrophages. See e.g., Fernandez-Soto, P., et. al. Mechanism of catalysis and inhibition of Mycobacterium tuberculosis SapM, implications for the development of novel antivirulence drugs. Sci Rep 2019, 9, 10315 and Fernández-Soto, P., et al. Discovery of uncompetitive inhibitors of SapM that compromise intracellular survival of Mycobacterium tuberculosis. Sci Rep 2021, 11, 7667. incorporated herein by reference.

In one example embodiment, the target protein is ESAT-6. M tb. produces ESAT-6 to mediate regulation of host immune responses. ESAT-6 directly inhibits T cell IFN-γ production by activation of p38 MAPK and indirectly through reprogramming of antigen presenting cells to produce less IL-12p70, an essential IFN-γ stimulating cytokine. Jung, B G., et al. Early Secreted Antigenic Target of 6-kDa of Mycobacterium tuberculosis Stimulates IL-6 Production by Macrophages through Activation of STAT3. Sci Rep 2017, 7, 40984.

In one example embodiment, the target protein is Rv2966c. Rv2966c is a RsmD-like methyltransferase, which carries out the transferase function through its N-terminal domain. This protein has been identified as a potential therapeutic target for its role in the function of M. tb and a conserved subunit similar to that of ATP binding pocket of kinases. The highly conserved subunit is called S-adenosyl-methionine (SAM). In one example embodiment, the Rv2966c binding moiety targets a SAM subunit. See e.g. Kumar A., et al. “Structural and Functional Characterization of Rv2966c Protein Reveals an RsmD-like Methyltransferase from Mycobacterium tuberculosis and the Role of Its N-terminal Domain in Target Recognition” J Biol Chem. 2011, 286(22): 19652-19661 and Copeland, R., et al. “Protein methyltransferases as a target class for drug discovery.” Nat Rev Drug Discov 2009, 8, 724-732, incorporated herein by reference.

Uridine 5′-monophosphate kinase (UMPK) catalyzes the reversible transfer of the γ-phosphoryl group from ATP to UMP in the presence of a divalent cation, usually Mg2+. Sequence comparisons of bacterial UMPKs show they do not resemble UMPKs from other organisms. Furthermore, bacterial UMPKs are specific for UMP and exist in solution as stable homohexamers. See e.g. Rostirolla D. C., et al. “UMP Kinase from Mycobacterium Tuberculosis: Mode of Action and Allosteric Interactions, and Their Likely Role in Pyrimidine Metabolism Regulation.” Archives of Biochemistry and Biophysics 2011, 505 (2), 202-212.

In one aspect, the method comprises treating infection by a pathogen as a result of PsA. PsA is a gram-negative, rod-shaped bacterium that commonly causes nosocomial pneumonia. In an example embodiment, the chimeric small molecule target binding moiety targets the pathogen PsA and is Colistin. Colistin, a polymyxin, is sulfomethylated and naturally ineffective antibiotic. Within the body at physiological temperature and pH, Colistin is hydrolyzed and becomes active. Colistin binds to phospholipids and disrupts the bacterial cell membrane. See e.g. Levin A. S., et al. Intravenous Colistin as Therapy for Nosocomial Infections Caused by Multidrug-Resistant Pseudomonas aeruginosa and Acinetobacter baumannii, Clinical Infectious Diseases 1999, 28, (5), 1008-1011. Colistin binds to lipopolysaccharide in the bacterial outer membrane, and also targets lipopolysaccharide in the cytoplasmic membrane. See, e.g. Sabnis et al., 2019, Colistin kills bacteria by targeting lipopolysaccharide in the cytoplasmic membrane, eLife doi:10.7554/eLife.65836.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a CRISPR-Cas system or component thereof described in greater detail elsewhere herein. In some embodiments, the pharmaceutical formulation can include, such as an active ingredient, a CRISPR-Cas polynucleotide described in greater detail elsewhere herein. In some embodiments, the pharmaceutical formulation can include, such as an active ingredient one or more modified cells, such as one or more modified cells described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a cancer or a symptom thereof. In some embodiments, the subject in need thereof has or is suspected of having, a neurobiological disease or disorder, a psychiatric disease or disorder, a cancer, an autoimmune disease or disorder, a thrombosis disease, a heart disease, a kidney disease, a lung disease, or a blood vessel disease, or a combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatoi anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g. a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In one example embodiment where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route.

Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, MD, 2000, and “Pharmaceutical dosage forms and drug delivery systems”, 6th Edition, Ansel et al., (Media, PA: Williams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In one example embodiment where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g. micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g. 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g. within seconds or just a few minutes apart), where the intent is that the formulations be administered together at the same time.

### Delivery and Administration

Methods for modifying a target of interest comprises administering or delivering or otherwise contacting a cell via one or more methods known in the art, including without limitation, microinjection, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the composition is introduced into an embryo by microinjection. The compositions may be microinjected into the nucleus or the cytoplasm of the embryo.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these aspects are within the ambit of the skilled artisan. In the field of active targeting, there are a number of cell-, e.g., tumor-, specific targeting ligands.

Also as to active targeting, with regard to targeting cell surface receptors such as cancer cell surface receptors, targeting ligands on liposomes can provide attachment of liposomes to cells, e.g., vascular cells, via a noninternalizing epitope; and, this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bilayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirus or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also as to active targeting, a lipid entity of the invention can be multifunctional, i.e., employ more than one targeting moiety such as CPP, along with Tf, a chimeric small system; e.g., a combination of Tf and poly-L-arginine which can provide transport across the endothelium of the blood-brain barrier. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm. With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG (SEQ ID NO: 44) such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention., e.g., with PEGylation. Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix. Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD. Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer). The targeting moiety can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in example embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)). Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes. The invention also comprehends redox-triggered delivery: The difference in redox potential between normal and inflamed or tumor tissues, and between the intra- and extra-cellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfide-to-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl) phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment. Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln) (SEQ ID NO: 45) can be incorporated into a linker, and can have antibody targeting, e.g., antibody 2C5. The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

Also as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and histidine-rich peptides have the ability to fuse with the endosomal membrane, resulting in pore formation, and can buffer the proton pump causing membrane lysis.

Also as to active targeting, cell-penetrating peptides (CPPs) facilitate uptake of macromolecules through cellular membranes and, thus, enhance the delivery of CPP-modified molecules inside the cell. CPPs can be split into two classes: amphipathic helical peptides, such as transportan and MAP, where lysine residues are major contributors to the positive charge; and Arg-rich peptides, such as TATp, Antennapedia or penetratin. TATp is a transcription-activating factor with 86 amino acids that contains a highly basic (two Lys and six Arg among nine residues) protein transduction domain, which brings about nuclear localization and RNA binding. Other CPPs that have been used for the modification of liposomes include the following: the minimal protein transduction domain of Antennapedia, a Drosophila homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, multipara, a wasp venom peptide; VP22, a major structural component of HSV-1 facilitating intracellular transport and transportan (18-mer) amphipathic model peptide that translocates plasma membranes of mast cells and endothelial cells by both energy-dependent and -independent mechanisms. The invention comprehends a lipid entity of the invention modified with CPP(s), for intracellular delivery that may proceed via energy dependent micropinocytosis followed by endosomal escape. The invention further comprehends organelle-specific targeting. A lipid entity of the invention surface-functionalized with the triphenyl phosphonium (TPP) moiety or a lipid entity of the invention with a lipophilic cation, rhodamine 123 can be effective in delivery of cargo to mitochondria.

DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

An embodiment of the system may comprise an actively targeting lipid particle or nanoparticle or liposome or lipid bilayer delivery system; or a lipid particle or nanoparticle or liposome or lipid bilayer comprising a targeting moiety whereby there is active targeting or wherein the targeting moiety is an actively targeting moiety. A targeting moiety can be one or more targeting moieties, and a targeting moiety can be for any desired type of targeting such as, e.g., to target a cell such as any herein-mentioned; or to target an organelle such as any herein-mentioned; or for targeting a response such as to a physical condition such as heat, energy, ultrasound, light, pH, chemical such as enzymatic, or magnetic stimuli; or to target to achieve a particular outcome such as delivery of payload to a particular location, such as by cell penetration.

It should be understood that as to each possible targeting or active targeting moiety herein-discussed, there is one example embodiment of the invention wherein the delivery system comprises such a targeting or active targeting moiety.

Further embodiments are illustrated in the following Examples which are given for illustrative purposes only and are not intended to limit the scope of the invention.

### Methods of Screening

Methods of screening for the combination of the protein binding moieties to be provided in the chimeric small molecule are provided herein. In one embodiment, the methods of screening/identifying binders of a protein target substrate. In one embodiment, the modifying polypeptide binding moiety is a modifying polypeptide inhibitor of an oncogenic fusion of a modifying polypeptide target substrate. By way of example, reference in the screening embodiment described screening Abl kinase binders for BCR-ABL; however, the screening method described is applicable to identification of kinase binders of other target substrates utilized in chimeric small molecules detailed herein. In example methods of screening, binders, for example inhibitors of BCR-ABL are identified based on the binding location on Abl.

The methods of nucleic acid analysis can be utilized for screening of chemical libraries, and to identify additional binders for use within the context of the embodiments disclosed herein.

In some embodiments, the disclosed methods can be used to screen chemical libraries for agents that bind modifying polypeptides and modulate activation or inactivation of the same. By exposing cells, or fractions thereof, tissues, or even whole animals, to different members of the chemical libraries, and performing the methods described herein, different members of a chemical library can be screened for their effect on modifying polypeptide binding and activation state.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

### Screening for Chimeric Small Molecules

A further aspect of the invention relates to a method for identifying a chimeric small molecule (e.g., bifunctional molecules) capable of modulating one or more phenotypic aspects of a cell or cell population as disclosed herein, comprising: a) applying a candidate chimeric small agent to the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells (e.g., an inflammatory phenotype or suppressive immune phenotype). In one example embodiment, steps can include administering candidate modulating agents to cells, detecting identified cell (sub) populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation-modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

After the chimeric small molecule is applied, a representative cell sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

### Computer Aided Electrophilic Reactive Linkers Design

Computer aided design (CAD) in the context of electrophilic reactive linkers comprises of computational methods to develop electrophilic reactive linkers for chimeric small molecules. Generally, CAD involves generating or obtaining a library of electrophilic reactive linkers for screening methods or Artificial Intelligence/Machine Learning (AI/ML) training. CAD methods comprising screening libraries may involve first selecting an enzyme, an enzyme binding moiety, a target binding moiety, and optionally a linker such as those described in the Linker section above. CAD screening methods may then utilize a generative algorithm to produce one or more chimeric small molecules with an electrophilic reactive linker designed to react with the enzyme and bind to a target. For example, a graph-based generative model may be used wherein the three-dimensional space is used to select an electrophilic reactive group bound to the chimeric small molecule that are within a defined distance to the nucleophile on the enzyme. In another example, the generative model may use natural language processing. For example, SMILES notation comprises embedded structural patterns and can be recognized by the generative model. These structural word patterns are then used to generate chimeric small molecules comprising electrophilic reactive groups that can bond to the desired enzyme.

CAD methods comprising AI/ML may comprise any regression model (e.g., neural networks) or classification network (e.g., Random Forest) to generate chimeric small molecules with an electrophilic reactive group. AI/ML CAD methods may be trained on the electrophilic reactive linker libraries, enzymes, targets, and chimeric small molecules. Trained AI/ML CAD methods can then take as input the target or the target and the enzyme. The AI/ML CAD model can then generate a chimeric small molecule capable of binding to the target comprising an electrophilic reactive linker that bonds to the enzyme. See e.g., Tunjic, T. M., Weber, N. and Brunsteiner, M., 2023. Computer aided drug design in the development of proteolysis targeting chimeras. Computational and Structural Biotechnology Journal. In example embodiments, the electrophilic reactive group is generated by CAD.

Further embodiments are illustrated in the following Examples which are given for illustrative purposes only and are not intended to limit the scope of the invention.

It is considered the electrophilic reactive linkers, functional moieties, and components thereof may and can be modified, both chemically and physically, to substitute, remove, or add components described herein or known in the art to arrive at the claimed subject matter. In particular, it may be that components of the claimed invention, for example, are illustrated with complete or filled valences in which there appears to be no specific connection point between one component and another component of the claimed invention. However, between the example constructs described herein and the ordinary skill in the art, one can readily assemble the compounds claimed. A lack of an open bonding position should not be construed as an intended closed system. Instead, the terminal groups of compounds described herein are considered as possible bonding positions to arrive at the claimed invention.

### EXAMPLES

### Example 1—Chimeric Small Molecules

### Harnessing Enzyme Inhibitors to Build Chimeras

Exemplary chimeric small molecules are formed by joining a kinase binder with a binder of a target protein-of-interest. Here, Applicants used the inhibitor-directed site-selective fast labeling of the kinase with the target binding moiety. Inhibitors tethered with a chemoselective electrophilic reactive group exhibit site-specific labeling of a side chain nucleophilic residue proximal to the inhibitor binding site, this approach has worked for several inhibitors. To neutralize the inhibitor following labeling, Applicants used bio-orthogonal handles (e.g., using cyclopropyl or azide) that do not perturb the binding of the inhibitor to the kinase. Upon completion of the proximity-induced labeling reaction, the cells will be treated with a large reactive group (e.g., tetrazine, cyclooctyne) whose conjugation will prevent the inhibitor from binding to the kinase, thereby deactivating the inhibitor. Applicants leveraged readily available inhibitors with low residence time (high k_{off}) to allow rapid labeling and quenching.

### Development and Application of Kinase-Binder Platform

In this work, Applicants developed and applied the novel kinase binder platform to find binders to label the kinases via proximity-induced labeling. Here, Applicants turned kinase inhibitors into labelers with the trifunctional molecule (kinase inhibitor-lysine reactive group-M.tb protein targeting group). The molecules bind to kinases via the kinase inhibitor, proximal lysines react with the lysine-reactive group (e.g., N-acyl-N-alkyl sulfonamide or NASA), and expel the kinase inhibitor, leaving the kinase tagged with an M.tb binder. The kinases, then covalently labeled with an M.tb binder, will hyper- and/or neo-phosphorylate the M.tb proteins, leading to HLA-display and an immune response. The kinase inhibitor contains a small bio-orthogonal group (e.g., cyclopropenyl, azide, tetrazine) that does not perturb the binding of the inhibitor to the kinase, and the inhibitor is quenched (e.g., with cyclooctyne), so the bulky group prevents the inhibitor from binding to the kinase. Applicants optimized NASA (covalent labeling) chemistry (see below) and optimized quenching the inhibitor (see below). Applicants focused on the NASA electrophilic reactive group since it is one of the most rapid cell-compatible proximity-induced labeling reagents, which was successfully used for labeling of PKC kinase by Applicants.

### Design and Optimization of NASA Chemistry for 5 Kinase Inhibitors

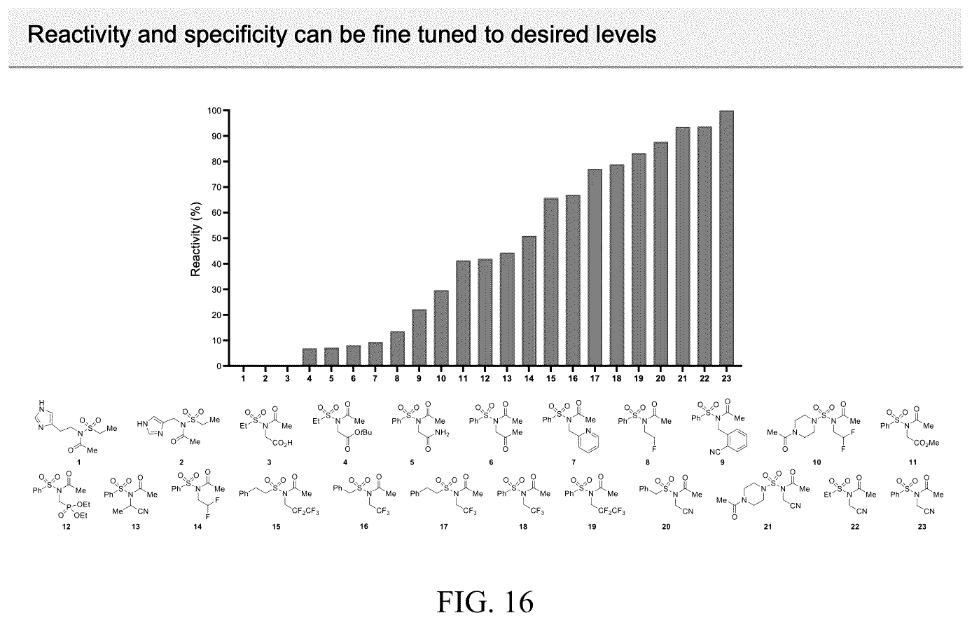

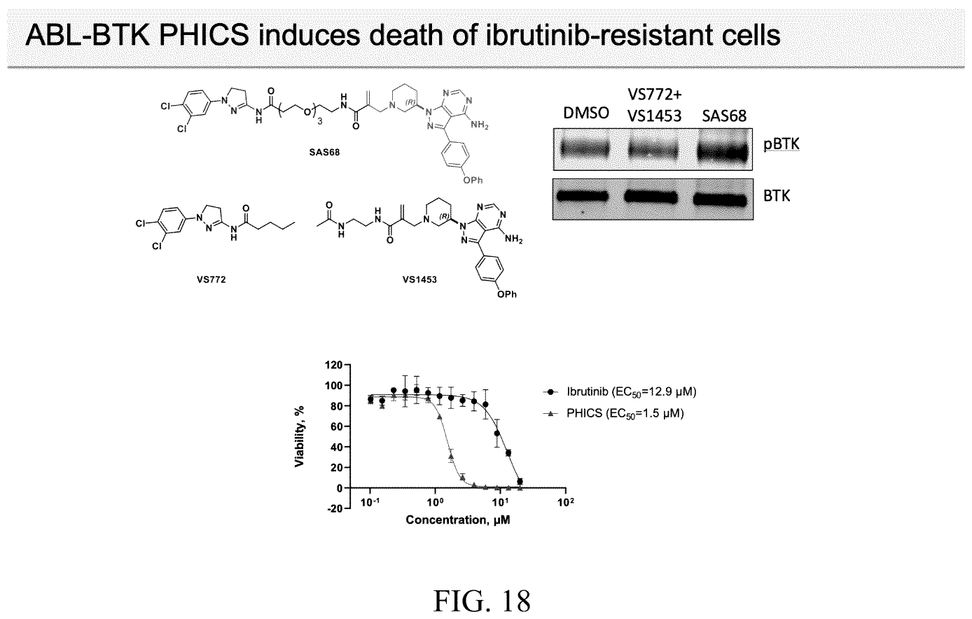

Applicants selected five kinases to optimize the NASA chemistry to label kinases with an inhibitor: MAPK, EGFR, ABL, CDK8, and P13K (FIG. 18). These kinases and inhibitors were chosen based on the following criteria (1) high kinase abundance in macrophages, (2) well-established binders with nanomolar activity, available crystal structure, and low residence time to ensure efficiency of the deactivation step, (3) binder should accommodate a small biorthogonal handle without affecting its binding potency/residence time (4) high density of lysines on the kinase close to the binding pocket, (5) labeling of kinase should not interfere with its enzymatic activity. For example, to design a probe for labeling of p38α MAPK, Applicants have chosen the following three binders: SB203580, Skepinone-B and Sorafenib with residence times 5, 150 and 88 seconds respectively (FIG. 18). The crystal structure of SB203580 (PDB: 3GCP) shows that linker attachment at the sulfoxide should not affect binding, and lysines (K15, K54, K66, K152, K165) are in close proximity to the binding pocket. Docking studies show that attachment of azide in ortho position to the fluoro group does not interfere with binding; however, the product of click reaction does not bind to p38α. Similar analysis of crystal structures for Skepinone (PDB: 3QUE) and Sorafenib (PDB: 3HEG) were performed. Gefitinib, Imatinib, Idelasilib and compound 5 were selected as binders of EGFR, ABL, PI3K and CDK8, respectively, due to their relatively low residence time (7s-17 min) and high availability of lysines in the close proximity to binding pockets (6 Lys for EGFR, 10 Lys for ABL, 6 Lys for CDK8, and 10 Lys for PI3K). For all of the inhibitors shown in FIG. 16, Applicants optimized labeling by creating trifunctional probes with: kinase binder-NASA group-coumarin and measure labeling via in-gel fluorescence of cell lysate after treatment with probes. Representative examples of fluorescent probe and mechanism of labeling are shown in FIG. 17A. As an alternative to NASA, dibromophenyl benzoate and N-sulfonyl pyridone electrophilic reactive groups will be considered. Applicants chose binders with 3 linkers per kinase (5 kinases total) to synthesize coumarin probes with and without the orthogonal binding group (15 compounds×2 variations=30 compounds), prioritizing the best 3 kinase-inhibitor pairs.

### Design and Optimize Bioorthogonal Chemistry for 5 Kinase Inhibitors

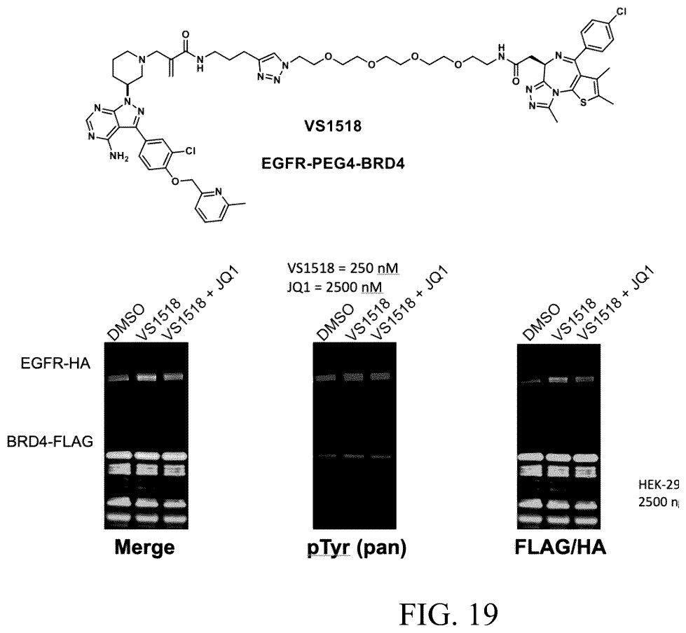

Applicants focused on click reaction between azide (substituent on kinase inhibitor) and cyclooctyne (quenching reagent) as a deactivating step. Mechanism of inhibitor deactivation is illustrated in FIGS. 17 and 19 where click renders the kinase inactive. Alternatively, inverse Diels-Alder reaction between tetrazine (bioisostere of phenyl) and trans-cyclooctene or cyclopropene will be considered. To optimize biorthogonal chemistry, various quenching reagents will be added to cells pretreated with optimized probes. Reaction conditions for the quenching step will be varied (concentration of coupling molecule, incubation time) and the activity of labeled kinases towards their natural substrates will be evaluated. Applicants measured if the kinase is quenched by looking at the kinase-substrate phosphorylation. If the kinase inhibitor is quenched, then the kinase phosphorylation of its substrate will be returned to basal levels.

### Target Selection: Immune Evasion is a Hallmark of PsA and M.tb.

The gram-negative pathogen PsA remains a serious human health threat with increasing instances of antibiotic-resistance and immune-system evasion. The outer-membrane of PsA acts as a physical barrier for antibiotics and hinders recognition by the immune system. Upon initial infection, the bacterium secretes alkaline protease and elastase, which degrade the complement protein C3b. Moreover, lipopolysaccharide (LPS) variants can interfere with C3b deposition. During the late stages of infection, PsA forms biofilms that protect the bacteria from complement-mediated phagocytosis. The forced recruitment of complement proteins, antibodies, or macrophages to PsA at high, local concentrations using chimeras empowers the immune system to deactivate these pathogens.

M.tb. also evades the host immune system through multiple mechanisms. The bacteria enter macrophages by conjugating to the complement proteins and subsequently uses their ESX-1 apparatus to secrete proteins that block endosome acidification and the immune system. For example, M.tb. attenuates antigen processing and MHC-II expression, secretes ESAT-6 to aid in phagosomal escape and intracellular survival and secretes protein tyrosine phosphatase A (PtpA) and mammalian cell entry protein 3E (Mce3E) to suppress the innate immune responses. PtpA, PtpB and SapM (secreted acid phosphatase) act on H⁺-V-ATPase and phosphatidylinositol, respectively, to prevent the phagosome acidification and maturation. M.tb limits the autophagy initiation by secreting enhanced intracellular survival protein, which rapidly acetylates host dual proteins phosphatase (DUSP16) and mitogen-activated protein kinase phosphatase-7 (MKP-7).

Recently, Applicants demonstrated the ability of phosphorylation-inducing chimeric small molecules (PHICS) to hyper-phosphorylate a non-native substrate of the kinase. Protein hyperphosphorylation not only deactivates the protein, but it also appends neo-epitopes on the target, which can potentially be recognized by the immune system (via HLA display) to evoke a robust immune response. Since M.tb secretes its several key proteins (e.g., PtpA, PtpB, SapM, ESAT-6, Rv2966c) in macrophages, Applicants will develop PHICS against these targets and demonstrate clearance of the infected macrophages by T cells.

### PHosphorylation-Inducing Chimeric Small Molecules (PHICS)

Neo-phosphorylations (unobserved in the native cellular environment) can also alter protein structure and function, and evoke immune response. PHICS-mediated neo-phosphorylation on M.tb proteins may evoke a strong immune response against the pathogen-specific phosphopeptides, allowing deactivation and elimination of infected macrophages by the immune system. Several phosphorylation sites recruit ubiquitin ligase and signal degradation. As such, PHICS may enable the deactivation and degradation of targeted proteins. Finally, the proposal to incorporate kinase inhibitors using proximity-induced labeling chemistries will allow engagement of kinases that are highly abundant in macrophages, thereby increasing opportunities to induce a desired therapeutic outcome.

Applicants note that PHICS will complement PROTACs in multiple ways. For example, PHICS can have several target sites (Ser, Thr, Tyr, and His) while PROTACs target only lysine. The efficiency of PROTAC depends on the efficiency of ubiquitination, which is a complex process compared to phosphorylation. Ubiquitination is a multistep modification involving the appendage of a protein and often yields a heterogeneous mixture of poly-ubiquitinated species in substoichiometric amounts. Phosphorylation is relatively simple, involving the appendage of a small phosphoryl group, which does not concatenate to form chains. Finally, ubiquitination involves large complexes compared to those of kinases. Additionally, PHICS target M. tb secretory proteins and kinases abundant in macrophages. Therefore, immune-response induced by neo-phosphorylations will be more selective towards infected cells. Because of its catalytic nature, PHICS may produce more prolonged effects until the clearance of the infected cells. PROTAC may inhibit the activity of a M.tb protein by degradation, but it is envisioned that PHICS-induced immune response would destroy the infected cell, preventing a subsequent infection of surrounding cells.

TABLE 2
Relative stability and reactivity of NASA analogs and alternatives

| Compound | Hydrolysis^{a} | Hydrolysis^{b} | Reactivity with Lysine | Reactivity with peptide |
|---|---|---|---|---|
| VS1356 | 44% | 96% | 0% | 0% |
| VS1358 | 70% | 86% | 10% from NHS Ester | 11% from NHS Ester |
| VS1377 | — | — | 8% from NHS Ester | 0% |
| VS1364 | 81% | 89% | 0% | 0% |
| VS1366 | 100% | 100% | 87% from NHS Ester | 80% from NHS Ester |
| VS1387 | 100% | 100% | 85% from NHS Ester | NA |
| UD37 | 100% | 100% | Reactivity observed | Reactivity observed |
| UD38 | 50% | 91% | 62% from NHS Ester | 60% from NHS Ester |
| UD46 | | 100% | 48% NHS Ester | 53% from NHS Ester |
| UD51 | | 100% | 15% from NHS Ester | 12% from NHS Ester |
| UD60 | | 88% | 33% from NHS Ester | 49% from NHS Ester |
| UD69 | | 100% | 4% from NHS Ester | 7% from NHS Ester |

4 uL of 10 mM compound, 32 uL 50 mM HEPES + 4 uL 50 mM lysine or tripeptide. Incubated for 16 h.
^{b}For Stability: 4 uL of 10 mM compound, 32 uL 50 mM HEPES + 4 uL DMSO. Incubated for 16 h.
^{a}Incubated for 12 h.

In Table 2, Applicants validated the relative stability of NASA analogs and alternatives by monitoring the hydrolysis of the molecules in the presence of Lysine and model peptide. The reactivity of the NASA analogs/alternatives was then identified.

### Table 3. Ligand Binding Studies

Table 3 comprises of RCSB PDB identifiers for kinases used in ligand binding studies. The ligand in each row was predicted to bind to the corresponding kinase using 3D structural analysis of binding pockets located on the corresponding kinase.

### Example 2

Bifunctional molecules are extensively used to degrade proteins via induced-proximity effects, albeit newer applications beyond protein degradation are arising.^{10,7} The appendage of a phosphoryl group alters the protein's electrostatic surface topology, dynamics, and functions.^{6,7} Unsurprisingly, small molecules that block protein phosphorylation via kinase inhibition have had a transformative impact on basic science and medicine.⁸ The data suggest that PHICS that induce phosphorylation of any given protein-of-interest on-demand are useful in myriad scenarios. We recently reported the first examples of PHICS for Serine/Threonine phosphorylation using two kinases (AMPK, PKC)¹¹ and will shortly report induction of tyrosine phosphorylation and signal transduction using PHICS that recruits Abelson (ABL) kinase (manuscript in preparation).

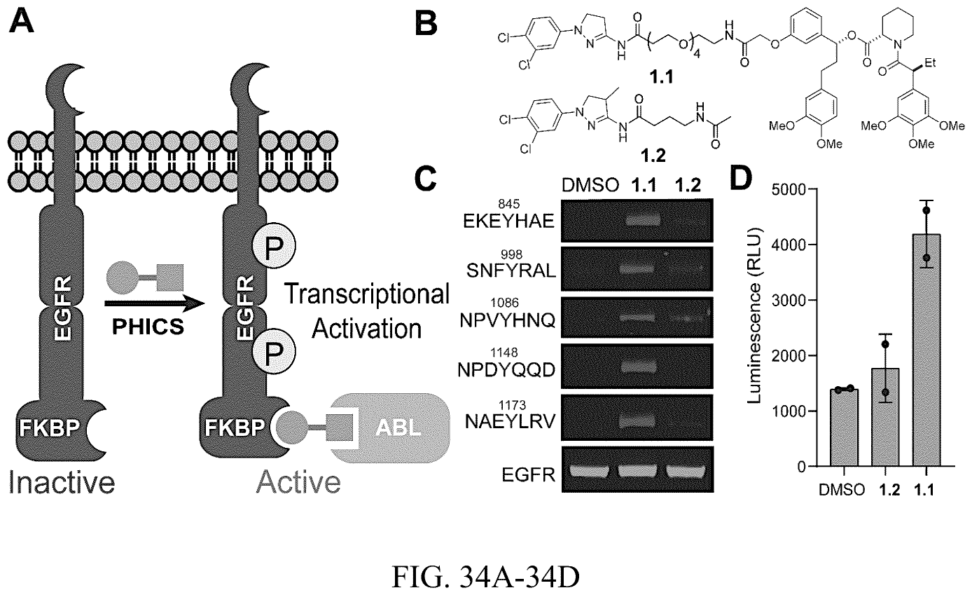

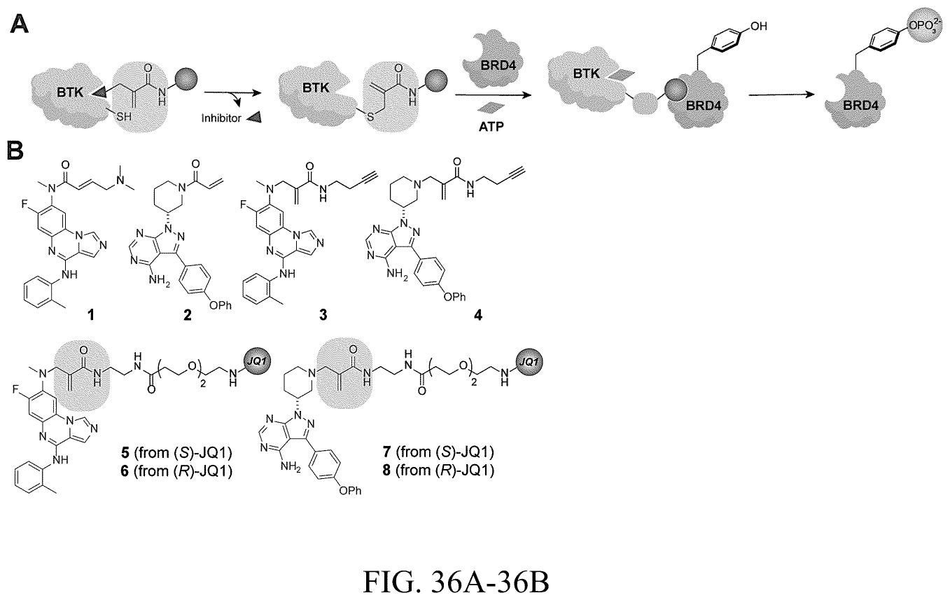

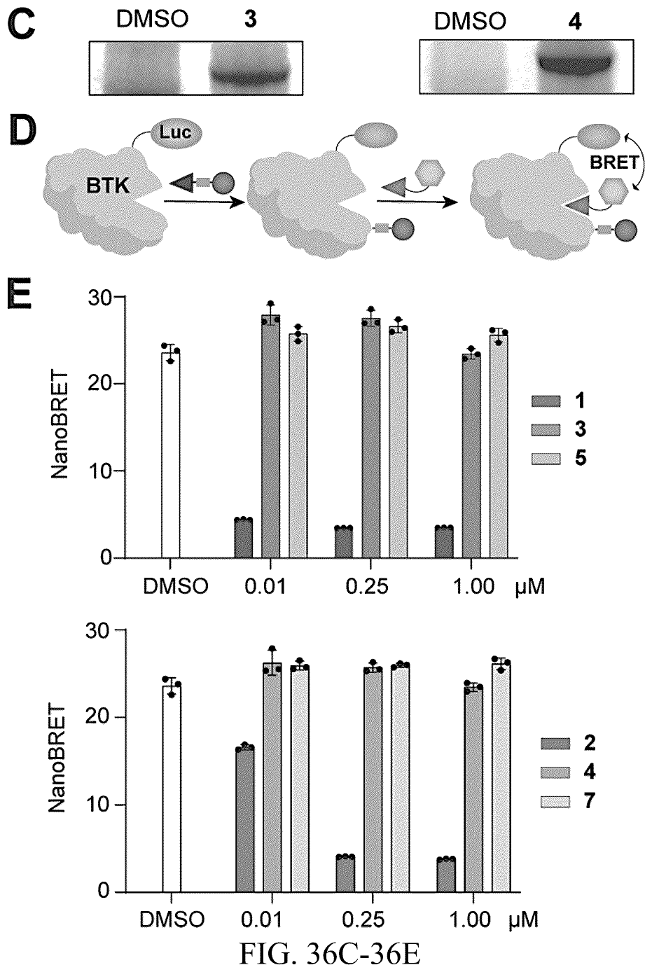

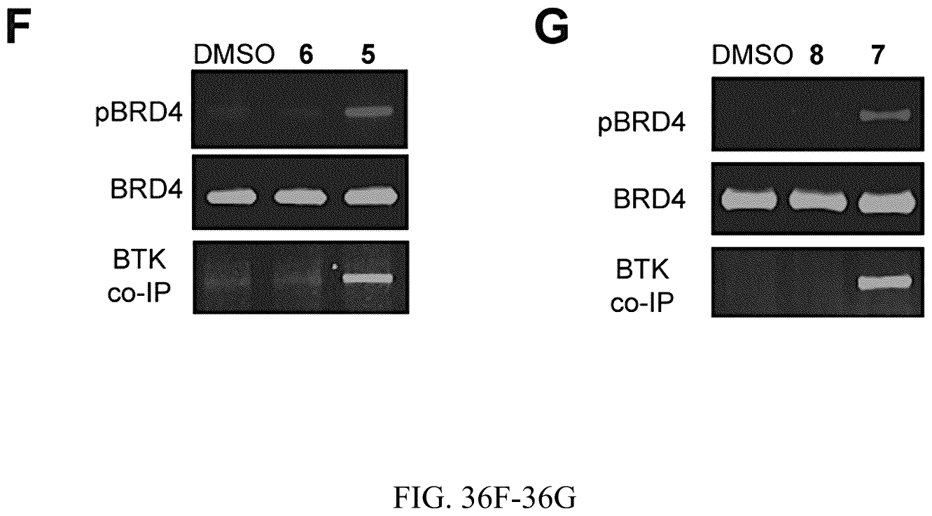

For example, using PHICS 1.1 (FIG. 34A, B) and engineered EGFR receptor bearing FKBPF36V, Applicants recruited Abelson kinase (ABL) to EGFR. This recruitment led to the induction of signaling relevant phosphorylations on EGFR by PHICS but not in DMSO or ABL binder 1.2 (FIG. 34C). Furthermore, Applicants used a previously described luciferase-based reporter gene assay with serum response element (SRE)¹² to monitor EGFR signaling activation and observed two-fold high luciferase activity for PHICS 1.1 compared to DMSO or ABL binder 1.2 (FIG. 34D). In another example of bioactivity, a PKC PHICS phosphorylated BCR-ABL at T735, a known binding site for 14-3-3 protein, resulting in BCR-ABL sequestration and death of Chronic Myeloid Leukemia (CML) cells (see FIG. 36B-D in ref²).

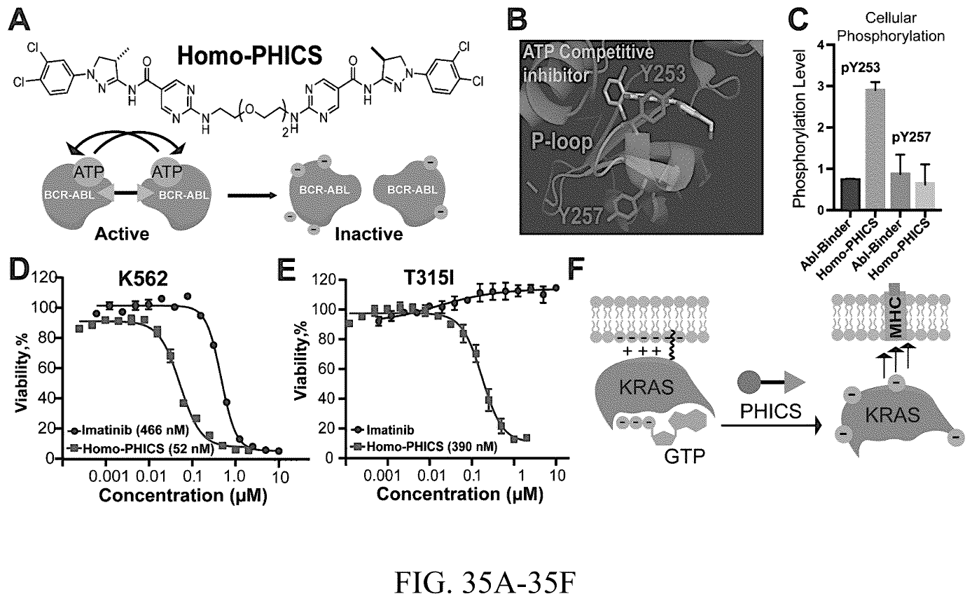

PHICS can also induce neo-phosphorylations that are not observed in native cellular environment⁹, and these phosphorylation can also alter protein structure and function,¹⁰ evoke an immune response,^{11,12} or affect the protein's interaction with other biomolecules, particularly with RNA/DNA that have a negatively-charged backbone.^{13,14} For example, Applicants found a dimer of ABL binder (i.e., Homo-PHICS, FIG. 35A) induced phosphorylation at Tyr253 on BCR-ABL in the ATP pocket (FIG. 35B, C), leading to inhibition of BCR-ABL and death of CML cells (FIG. 35D) even those with mutations that are resistant to known drug imatinib (FIG. 35E).

The inability to harness the widely available kinase inhibitor to generate PHICS has greatly limited the technology and its applications. Furthermore, to demonstrate the utility of PHICS, Applicants have chosen oncogenic KRAS as the target for multiple reasons. First, KRAS is a high-value therapeutic target for which inhibitors¹³ are emerging (e.g., MRTX849, AMG510, MRTX1133), but so are resistance to these inhibitors¹⁴ and several studies suggest that resistance mechanisms of bifunctional molecules are different from traditional enzyme inhibitors. Second, by altering the electrostatic surface topology/folding, the neo-phosphorylation will interfere with the GTP binding/membrane translocation of KRAS (FIG. 35F). Applicants have identified ˜23 Ser/Thr near/on the GTP binding pocket/polybasic region of KRAS that can be targeted, including Ser 17 that coordinates with Mg²⁺ or Thr35 makes contacts with GTP.¹⁵ Third, KRAS mutants are known to be HLA displayed^{8,9} and neo-PTMs can enhance the immunogenicity of these neo-antigens. Finally, KRAS is heavily post-translationally modified and interacts with several proteins; neo-phosphorylations may perturb the induction of endogenous PTMs and protein-protein interactions, offering additional avenues to inhibit KRAS activity.

### Aim 1. Development and Application of PHICS Using Cysteine-Based Group Transfer Chemistry

A PHICS based on BTK inhibitors: Applicants developed²⁵ PHICS from a kinase inhibitor in which a nucleophile (e.g., cysteine) near the inhibitor-binding pocket can initiate a group transfer chemistry²⁶⁻²⁹ resulting in appending the target-protein binder to the kinase while releasing the inhibitor from the active site (FIG. 36A). The kinase binder in such cysteine-tagging PHICS can be derived from abundantly available acrylamide-based inhibitors, where the amide nitrogen is often aryl amine (1, FIG. 36B) or less frequently aliphatic amine (2, FIG. 36B). Using these BTK inhibitor scaffolds, Applicants synthesized alkyne-containing analogs 3 and 4 by installing the cleavable methacrylamide linker (FIG. 36B).

To confirm BTK labeling, HEK293T cells transiently expressing BTK were treated with alkyne-containing compounds 3 or 4, and subsequent click chemistry with dye Cy5-N3 in lysate showed BTK labelling in-gel fluorescence (FIG. 36C). Next, BRD4-BTK PHICS 5 (aryl amine scaffold) and 7 (aliphatic amine scaffold) wherein the BTK inhibitor scaffold is connected to BRD4 binder, (S)-JQ1, via cleavable methacrylamide linker were synthesized (FIG. 36B). To demonstrate that cleavable methacrylamides do not block ATP-binding pocket, Applicants used a reported assay based on Bioluminescence Resonance Energy Transfer (BRET) between a nanoluciferase (nanoLuc) and a fluorophore probe (tracer) that binds to the ATP pocket;³⁰ a higher occupancy of this ATP pocket by the inhibitor will prevent the binding of tracer and lower the BRET signal (FIG. 36D). Observed BRET ratios demonstrated that BTK inhibitors 1 and 2 dramatically lower BRET signal, while PHICS 5 or 7 or their alkyne analogs 3 and 4 did not (FIG. 36E). In congruence with these findings, Applicants observed higher autophosphorylation of BTK with PHICS compounds compared to the parent inhibitors 1 or 2 (data not shown). Finally, Applicants synthesized inactive controls of PHICS 5 and 7 (i.e., 6 and 8, FIG. 35B) using (R)-JQ1, which is a poor binder of BRD4 and tested these controls alongside 5 and 7 for their ability to induce phosphorylation of a cytosolically localized BRD4 (lacks NLS) by BTK as described previously.² As expected, much higher BRD4 phosphorylation was observed in the presence of 5 (FIG. 36F) or 7 (FIG. 36G) than that with inactive controls, 6 or 8 (FIG. 36F-G). Applicants also observed significantly higher levels of co-immunoprecipitated BTK-FLAG in 5 or 7-treated samples compared to inactive controls, 6 or 7, suggesting ternary proximity induction by PHICS.

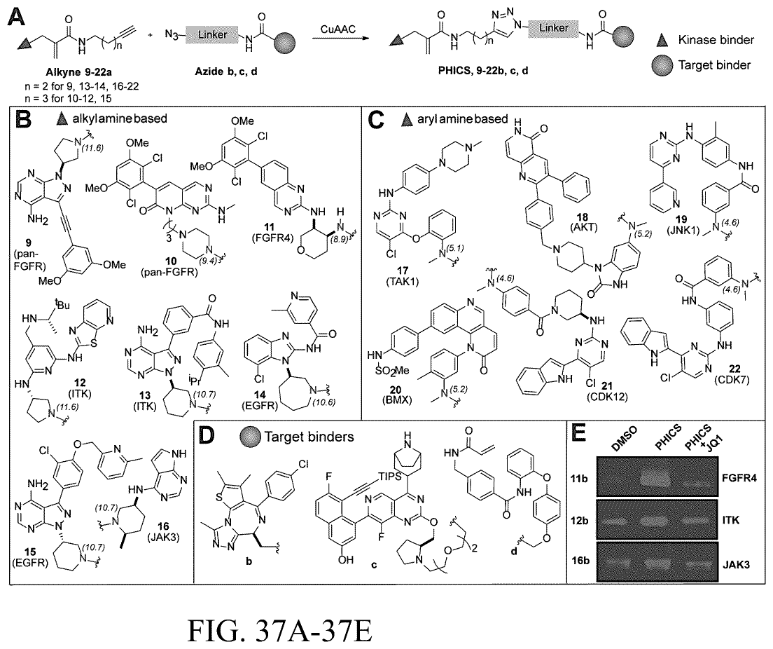

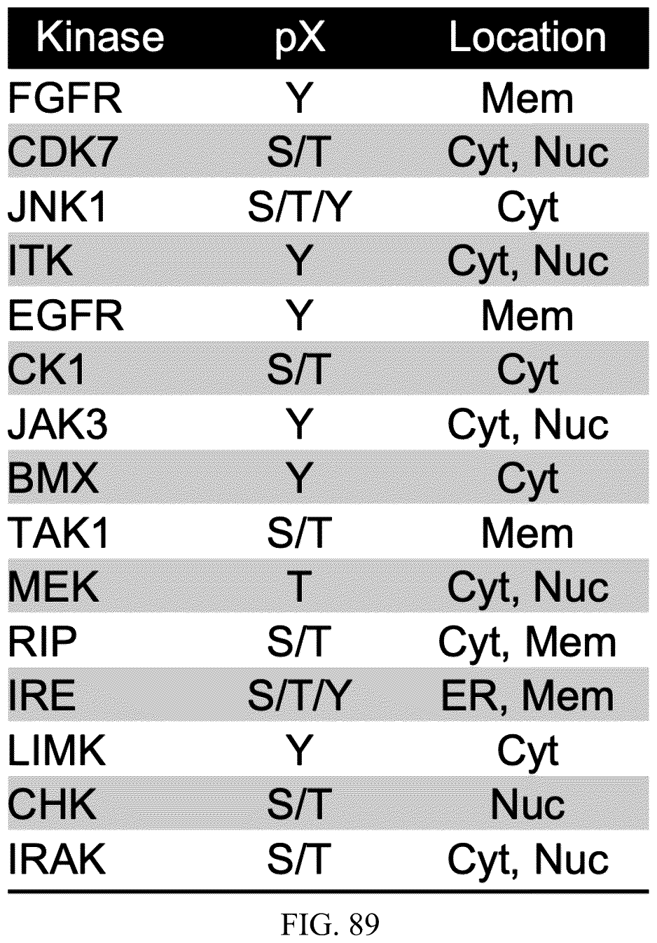

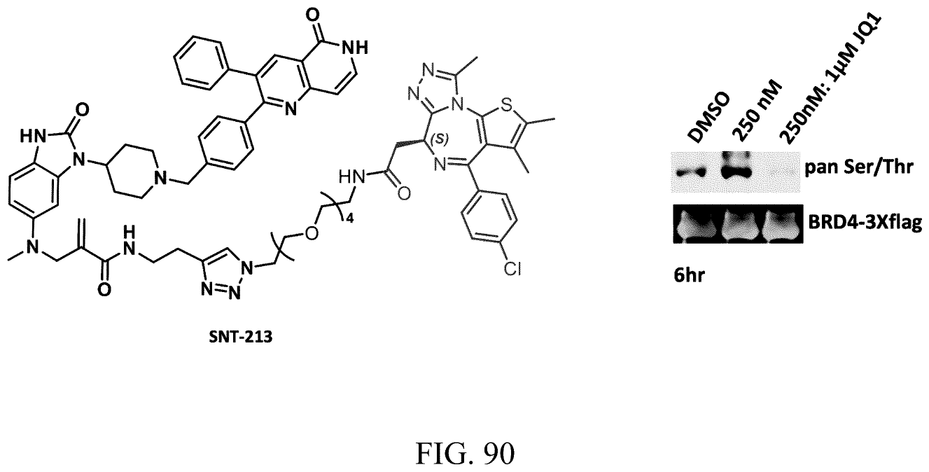

C.1.1.2. Synthesis and evaluation of cysteine-targeting PHICS for diverse kinases: To demonstrate generalizability and leveraging several known acrylamide-based kinase inhibitors, Applicants synthesized PHICS for a diverse set of kinases (FIG. 89, FIG. 37) that differ in specificity (i.e., Ser/Thr vs. Tyr), cellular localization [e.g., cytosolic (cyt), nuclear (nuc), plasma membrane (mem), or endoplasmic reticulum (ER)], are overexpressed in diverse diseases, and phosphorylate diverse sequences. For example, FGFR phosphorylates pYxxQ motifs while BMX affects pYpY. Similarly, CDK phosphorylates [S/T*]Px[K/R] while CK phosphorylates D/E-D/E-D/E-X-X-S/T-φ or pS/pT-X-X-S/T-φ. Also, Applicants have chosen acrylamide-based kinase inhibitors with diverse pKa (˜5-12, shown in blue FIG. 37B, 37C) at the leaving nitrogen atom. Leaving amino groups with lower pKa (weaker bases) may not facilitate the abstraction of a proton from Cys thiol and will be a poor leaving group as opposed to amino groups with high pKa (strong bases). Applicants used previously reported inhibitors scaffolds for pan-FGFR^{31,14}, FGFR4¹⁵, ITK^{32,33}, EGFR^{34,35}, JAK3³⁶, TAK1³⁷, AKT³⁸, JNK1³⁹, BMX⁴⁰, CDK7⁴¹, and CKD12⁴² to synthesize their alkyne analogs and subsequent click chemistry with azide to generate the PHICS molecules using BRD4 as model neosubstrate (FIG. 37A, 37D). Using Ac-Cys-OMe as nucleophile, Applicants have successfully demonstrated release of leaving group in vitro with alkyne-bearing compounds 9-22a with most aryl amines reacting slowly compared to aliphatic amines (data not shown). Furthermore, using BRD4 as the model substrate, Applicants demonstrated PHICS-induced phosphorylation in HEK293T cells using FGFR, ITK, and JAK3 as the kinase (FIG. 37E). Comfortingly, these phosphorylations were decreased when cells were treated with competitor JQ1 in excess with PHICS.

C.1.2. Experimental Plan. C.1.2.1. Biochemical and cellular target engagement of kinases. Alkyne-bearing inhibitor compounds (FIG. 37A, 37B, 37C) with the Cys targeting warhead will be incubated with the respective kinases and conjugation will be assessed using in-gel fluorescence. Briefly, upon Cys labeling of the protein, copper-catalyzed click chemistry will be performed with azide-Cy5 and the extent of labeling will be directly visualized on the SDS-PAGE gel. Intact mass spectrometry will determine whether the conjugated protein formed an addition or elimination adduct with the compounds. Once the eliminated adduct mass is confirmed for the kinase, conjugated kinases will be tested for their activity using ADP-Glo™ assay, 1,2 which measures the amount of ADP produced from the kinase reaction. Following these biochemical studies, Applicants will use the alkyne probe to evaluate the on-target and off-target labeling of various kinases in HEK293T as demonstrated in FIG. 36C and we will assess the inhibitor occupancy of ATP pocket using NanoBRET assay. These studies will provide quantitative data on kinase's labeling efficiency and inhibition by alkyne compounds.

C.1.2.2. Cellular validation of PHICS using cytosolic BRD4 as neosubstrate. After the aforementioned studies, Applicants will focus on kinases with the highest labeling efficiency and lowest inhibition and generate BRD4 PHICS that will be tested in HEK293T cells as described in FIG. 36F-36G. Cells will be treated with active or inactive PHICS for ˜2 hrs and phosphorylation of the BRD4 will be monitored after immunoprecipitation, followed by immunoblotting with phosphor-S/T/Y antibodies. Co-immunoprecipitation of the kinase and the target in the presence of PHICS (JQ1 can be used as a competitor) will be attempted to confirm the complex formation.

C.1.2.3. Phosphorylation of KRAS and binary complex modeling. The BRD4 PHICS studies will identify kinases and ligands for KRAS G12D phosphorylation and examination of the effects of such phosphorylation on neoepitope display and downstream signaling. For the kinases ligands that successfully phosphorylate BRD4 above, we will synthesize PHICS using KRAS G12D inhibitor (FIG. 37D). Subsequently, we will perform ADP-Glo assay to determine target protein phosphorylation, leveraging the high-throughput nature of this assay. Next, Applicants will use previously reported AlphaScreen assay^{1,2} that quantifies complex formation in the presence of PHICS to identify the most optimal molecules for complex formation between kinase and target protein. The catalytic nature of these active PHICS molecules will be assessed by ADP-Glo assay. Western blotting will be performed to determine the phosphorylation of the target protein in the presence of PHICS (and KRAS binder as a competitor for control) using phospho-S/T/Y, or substrate motif antibodies. Mass spectrometry studies will be performed in the presence of PHICS to identify the neo-phosphorylation sites on KRAS G12D.

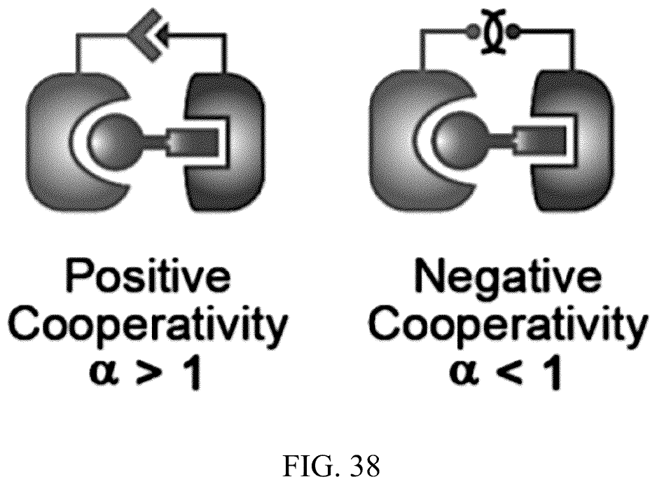

The protein-protein interactions between the kinase and KRAS G12D can enhance or attenuate the activity of PHICS molecules. Even if the binding affinities of the individual components comprising PHICS are very high, if the enforced orientation is incorrect then a productive complex will not be formed, and phosphorylation will not occur effectively. Applicants can alter the identity of the kinase/KRAS binder, linker attachment sites, length and identity of the linkers, and exit vectors—how the linker exits the protein—to design PHICS that can promote cooperativity (FIG. 38). To determine the nature of cooperativity, we will model the equilibria between the labeled kinase and KRAS. There are two systems of equilibria, non-cooperative equilibria, and cooperative equilibria. In non-cooperative equilibria (α=1), the terminal bodies (kinase and KRAS) are unable to interact with each other. In the cooperative equilibria, the interaction between these bodies is either enhance (termed positive cooperativity, a >1) or diminish (termed negative cooperativity, α<1).

C.1.2.4. Proteomic studies for off-target identification and HLA-peptidome analysis: Following the identification of optimum KRAS PHICS, Applicants will identify the off-target phosphorylation of not only the non-target proteins in cells but also on phosphorylation sites within the target protein that do not match the kinase substrate motif. Here, Applicants will use SILAC/TMT-based global phosphoproteomics where a given PHICS is incubated with “heavy,” 13C, 15N-bearing arginine and lysine residues, labelled 293T cells and compared to “light,” naturally abundant isotope, labelled cells treated with unjointed congeners. Changes in global phosphorylation levels of proteins will be monitored to determine PHICS specificity.⁴⁴

Applicants will follow the published protocol^{46,47} that identify HLA-associated phosphopeptides to analyze the neo-phosphorylation induced by PHICS. Briefly, phosphopeptides generated from proteolysis of aberrantly phosphorylated KRAS are loaded onto HLA class I molecules. HLA-associated peptide complexes are then shuttled to the cell surface and displayed to the immune system to elicit a specific cytotoxic T cell response. Here, FHIOSE cells will be stably transfected with soluble HLA (sHLA)-A*0201 construct with a VLDLr sequence as a downstream purification tag. Subsequently, cells will be incubated with PHICS and the sHLA/peptide complexes will be extracted using an immunoaffinity column coupled with anti-VLDLr antibody. Upon phosphopeptide-enrichment, samples will be subjected to MS/MS to identify their sequence.

### Aim 2. Development and Application of PHICS Using Lysine-Based Group Transfer Chemistry.

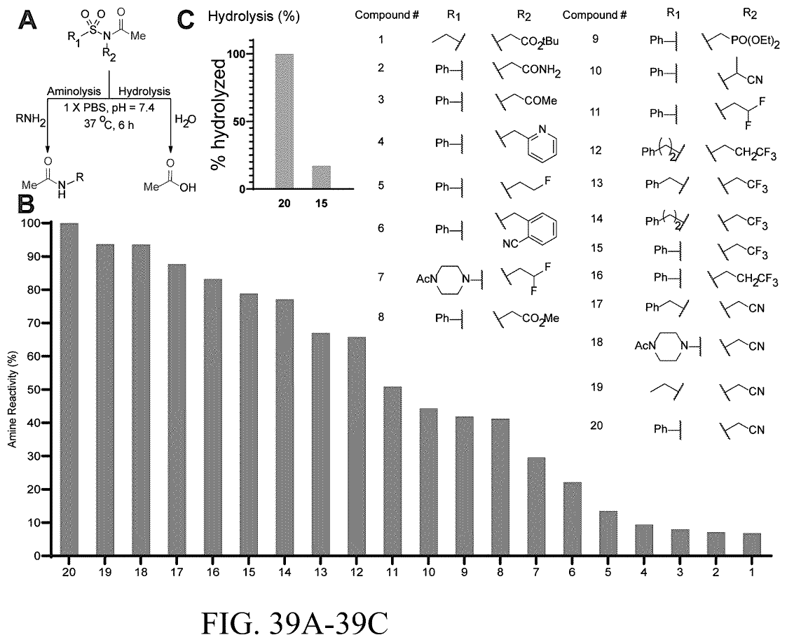

C.2.1. Preliminary studies. C.2.1.1. Development of optimum linker for lysine reactivity: Most of Lys targeting group transfer linkers suffer from poor reaction rates, lack of stability/selectivity, and bulky nature. N-acyl-N-alkyl sulfonamides (NASA) have recently gained interest as covalent warheads for lysine targeting due to their fast reaction rates towards lysine (k≈104 M⁻¹ s⁻¹)^{48,49} Despite these promising attributes, NASA is bulky (unfit to be used as a linker for bifunctional molecules that are already large), hydrolyzes rapidly (t_{1/2}≈5 h in PBS), and lack tunable analogs. Guided by physical organic chemistry, Applicants rationally designed NASA analogs with a small footprint, higher stability, and tunable reactivity. The NASA warheads follow a general structural motif as described in FIG. 39A. Traditionally R₁ is a phenyl ring that in original reports served as a convenient handle for attachment to a solid phase resin⁵⁰. Replacement of this ring with short aliphatic groups did not affect its reactivity towards lysine (compare 19 and 20 in FIG. 39B), permitting significant size reduction without loss of reactivity. The electrophilicity of the N-acyl group depends on the electron-withdrawing properties of the N-alkyl group (—R₂), allowing fine-tuning of amine reactivity and hydrolytic stability. Applicants synthesized a series of NASA derivatives (FIG. 39B) and identified the —CH2CF3 group as a superior alternative to the typically used-CH2CN by providing comparable rates towards aminolysis (compare compounds 15 and 20 in FIG. 39B) accompanied by a 5-fold decrease of hydrolysis rates (FIG. 39C). These studies also provided analogs to fine-tune reactivity (FIG. 39B) and effective molarity.

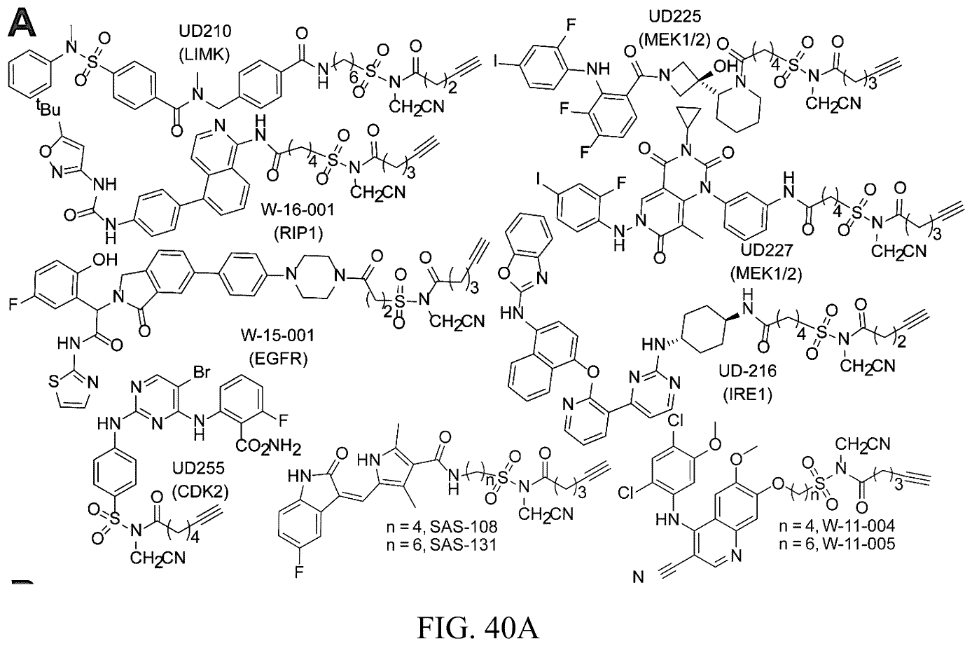

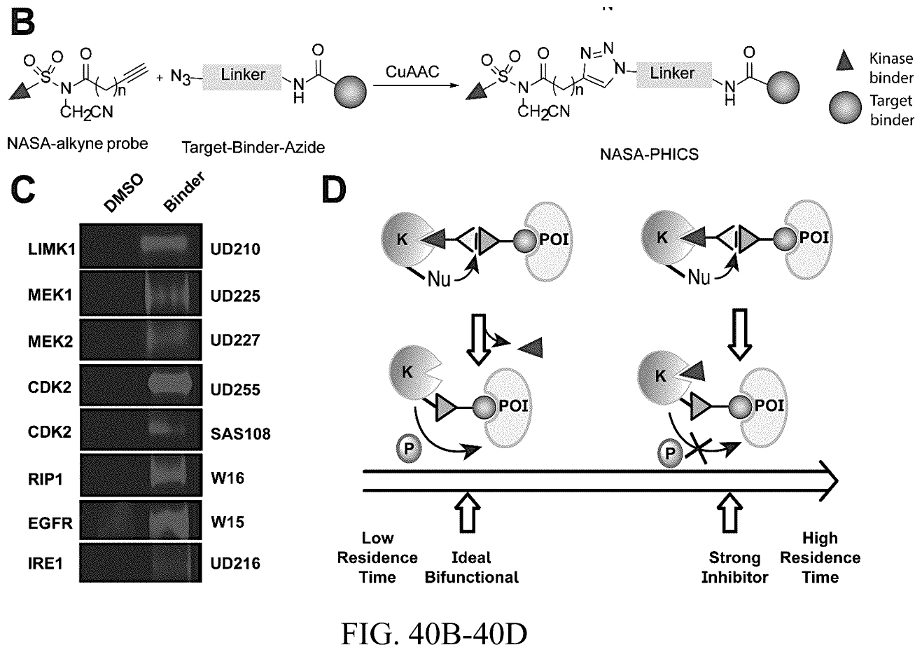

C.2.1.2. Synthesis and evaluation of lysine-targeting PHICS. Applicants designed and synthesized lysine targeting probes based on a variety of previously described high-affinity, allosteric kinase inhibitors (K_{d}<100 nM) (FIG. 40A) targeting LIMK⁵¹, MEK1/2⁵², RIP1⁵³, EGFR⁵⁴, IRE1⁵⁵ and CDK2⁵⁶. Crucial selection criteria for the parent kinase inhibitors were the availability of crystallographic data, which allowed for the rational incorporation of NASA linkers proximal to lysine residues. In addition, the inhibitors chosen for the lysine targeting PHICS bind to allosteric sites of the kinases, diversifying the potential neo-phosphopeptides that can be accessed. These molecules were used for the synthesis of Lys targeting-PHICS similarly to the Cys targeting ones by combining the alkyne probes described in FIG. 40A with the target binder azides, via click chemistry (FIG. 40B). In addition, the alkyne components were used to demonstrate the ability of the NASA kinase inhibitors to sufficiently label the corresponding kinases in in-vitro assays. Treatment of purified proteins with the appropriate probes under proximity-driven reactivity conditions (i.e., <10 μM) followed by click chemistry with dye Cy5-N3 demonstrated fluorescence labelling with probes over control treatment (FIG. 40C), validating our rational design approach and confirming presence of reactive Lys.

C.2.2. Experimental Plan. C.2.2.2. Biochemical and cellular target engagement of kinases (same as in C.1.2.2). The initial design of PHICS molecules is based on high-affinity kinase inhibitors (K_{d}<100 nM). Although this is desirable from a labelling perspective, subsequent kinase inhibition can reduce PHICS efficacy. Applicants will systematically study the impact of residence time and PHICS efficacy (FIG. 40D). For example, Sunitinib and Bosutinib (the middle bottom and right bottom in FIG. 40A) can bind to Ser/Thr and Tyr kinases with varying residence time (t).⁵⁷ Sunitinib for example, can bind to kinases with residence time ranging from seconds to minutes [t=5 seconds for CHK1, t=10 seconds for IRAK4, t=37 seconds for ITK, t=2.9 minutes VEGFR].

The size of some of the proposed PHICS molecules can potentially limit their cell permeability. However, Applicants would like to point out that their weight and clogP values are close to those of successful cell-permeable PROTACs and such permeability could be arising from receptor-mediated transport as has been demonstrated recently for many molecules.⁵⁸⁻⁶⁰ Applicants will adjust polarity by changing the nature of the linkers (alkyl vs. PEG, N-methylation of amide bonds), to assemble final PHICS molecules inside the cell via metal-free click-chemistry.⁶¹

The phosphorylation stoichiometry must be relatively high to elicit an immune response. The usage of pan-targeting kinase inhibitors in our PHICS design will circumvent this concern. A significant challenge in selectively detecting neo-phosphopeptides on HLA is the off-target phosphorylation events arising from kinase inhibitor moiety present in PHICS. To overcome this, Applicants will do time and dose titrations to determine an acute treatment of PHICS to avoid off-target effects. The dynamic nature of phosphorylation and dephosphorylation may limit the number of occurrences of neo-phosphorylation and neo-phosphopeptides on HLA display. As such, Applicants will use phosphatase inhibitors treated cells.

### REFERENCES FOR EXAMPLE 2

### Bibliography

(1) Phosphorylation-Inducing Chimeric Small Molecules. Siriwardena, S. U.; Munkanatta Godage, D. N. P.; Shoba, V. M.; Lai, S.; Shi, M.; Wu, P.; Chaudhary, S. K.; Schreiber, S. L.; Choudhary, A. J Am Chem Soc 2020, 142, 14052-14057
(2) Synthetic Reprogramming of Kinases Expands Cellular Activities of Proteins. Shoba, V. M.; Munkanatta Godage, D. N. P.; Chaudhary, S. K.; Deb, A.; Siriwardena, S. U.; Choudhary, A. Angew Chem Int Ed Engl 2022, 61, e202202770.PMC9527066
(3) Kinase inhibitors: the road ahead. Ferguson, F. M.; Gray, N. S. Nat Rev Drug Discov 2018, 17, 353-377
(4) Kinase drug discovery 20 years after imatinib. Cohen, P.; Cross, D.; Jänne, P. A. Nat Rev Drug Discov 2022
(5) Small Molecule Kinase Inhibitor Drugs (1995-2021): Medical Indication, Pharmacology, and Synthesis. Ayala-Aguilera, C. C.; Valero, T.; Lorente-Macías, Á.; Baillache, D. J.; Croke, S.; Unciti-Broceta, A. J Med Chem 2022, 65, 1047-1131
(6) The chemical biology of protein phosphorylation. Tarrant, M. K.; Cole, P. A. Annu. Rev. Biochem. 2009, 78, 797-825
(7) Bifunctional modalities for repurposing protein function. Modell, A. E.; Lai, S.; Nguyen, T. M.; Choudhary, A. Cell Chem Biol 2021, 28, 1081-1089
(8) Biochemical and functional characterization of mutant KRAS epitopes validates this oncoprotein for immunological targeting. Bear, A. S.; Blanchard, T.; Cesare, J.; Ford, M. J.; Richman, L. P.; Xu, C.; Baroja, M. L.; McCuaig, S.; Costeas, C.; Gabunia, K.; Scholler, J.; Posey, A. D., Jr.; O'Hara, M. H.; Smole, A.; Powell, D. J., Jr.; Garcia, B. A.; Vonderheide, R. H.; Linette, G. P.; Carreno, B. M. Nat Commun 2021, 12, 4365.PMC8285372
(9) Systematic discovery and validation of T cell targets directed against oncogenic KRAS mutations. Choi, J.; Goulding, S. P.; Conn, B. P.; McGann, C. D.; Dietze, J. L.; Kohler, J.; Lenkala, D.; Boudot, A.; Rothenberg, D. A.; Turcott, P. J.; Srouji, J. R.; Foley, K. C.; Rooney, M. S.; van Buuren, M. M.; Gaynor, R. B.; Abelin, J. G.; Addona, T. A.; Juneja, V. R. Cell Reports Methods 2021, 1, 100084
(10) The Rise of Molecular Glues. Schreiber, S. L. Cell 2021, 184, 3-9
(11) Phosphorylation-Inducing Chimeric Small Molecules. Siriwardena, S. U.; Munkanatta Godage, D. N. P.; Shoba, V. M.; Lai, S.; Shi, M.; Wu, P.; Chaudhary, S. K.; Schreiber, S. L.; Choudhary, A. Journal of the American Chemical Society 2020, 142, 14052-14057
(12) Luciferase Reporter Assay System for Deciphering GPCR Pathways. Cheng, Z.; Garvin, D.; Paguio, A.; Stecha, P.; Wood, K.; Fan, F. Curr Chem Genomics 2010, 4, 84-91.PMC3040460
(13) Direct small-molecule inhibitors of KRAS: from structural insights to mechanism-based design. Ostrem, J. M.; Shokat, K. M. Nat Rev Drug Discov 2016, 15, 771-785
(14) Advances in covalent kinase inhibitors. Abdeldayem, A.; Raouf, Y. S.; Constantinescu, S. N.; Moriggl, R.; Gunning, P. T. Chem Soc Rev 2020, 49, 2617-2687
(15) RAS-targeted therapies: is the undruggable drugged? Moore, A. R.; Rosenberg, S. C.; McCormick, F.; Malek, S. Nature Reviews Drug Discovery 2020, 19, 533-552
(16) Designing customized cell signalling circuits. Lim, W. A. Nat Rev Mol Cell Biol 2010, 11, 393-403
(17) The Age of Crosstalk: Phosphorylation, Ubiquitination, and Beyond. Hunter, T. Molecular Cell 2007, 28, 730-738
(18) Chemical genetic control of protein levels: Selective in vivo targeted degradation. Schneekloth, J. S.; Fonseca, F. N.; Koldobskiy, M.; Mandal, A.; Deshaies, R.; Sakamoto, K.; Crews, C. M. Journal of the American Chemical Society 2004, 126, 3748-3754
(19) Targeting steroid hormone receptors for ubiquitination and degradation in breast and prostate cancer. Rodriguez-Gonzalez, A.; Cyrus, K.; Salcius, M.; Kim, K.; Crews, C. M.; Deshaies, R. J.; Sakamoto, K. M. Oncogene 2008, 27, 7201-11
(20) Catalytic in vivo protein knockdown by small-molecule PROTACs. Bondeson, D. P.; Mares, A.; Smith, I. E. D.; Ko, E.; Campos, S.; Miah, A. H.; Mulholland, K. E.; Routly, N.; Buckley, D. L.; Gustafson, J. L.; Zinn, N.; Grandi, P.; Shimamura, S.; Bergamini, G.; Faelth-Savitski, M.; Bantscheff, M.; Cox, C.; Gordon, D. A.; Willard, R. R.; Flanagan, J. J.; Casillas, L. N.; Votta, B. J.; den Besten, W.; Famm, K.; Kruidenier, L.; Carter, P. S.; Harling, J. D.; Churcher, I.; Crews, C. M. Nature Chemical Biology 2015, 11, 611-U120
(21) Phthalimide conjugation as a strategy for in vivo target protein degradation. Winter, G. E.; Buckley, D. L.; Paulk, J.; Roberts, J. M.; Souza, A.; Dhe-Paganon, S.; Bradner, J. E. Science 2015, 348, 1376-1381
(22) Small-Molecule PROTACS: New Approaches to Protein Degradation. Toure, M.; Crews, C. M. Angew Chem Int Ed Engl 2016, 55, 1966-73
(23) Protein aggregation during overexpression limited by peptide extensions with large net negative charge. Zhang, Y.-B.; Howitt, J.; McCorkle, S.; Lawrence, P.; Springer, K.; Freimuth, P. Protein Expression and Purification 2004, 36, 207-216
(24) Charge substitution shows that repulsive electrostatic interactions impede the oligomerization of Alzheimer amyloid peptides. Guo, M.; Gorman, P. M.; Rico, M.; Chakrabartty, A.; Laurents, D. V. FEBS Letters 2005, 579, 3574-3578
(25) Bifunctional chimeric molecules for labeling of kinases with target binding moieties and methods of use thereof. Choudhary, A.; Shoba, V. Apr. 9, 2021
(26) Chemo- and Regioselective Lysine Modification on Native Proteins. Matos, M. J.; Oliveira, B. L.; Martínez-Sáez, N.; Guerreiro, A.; Cal, P.; Bertoldo, J.; Maneiro, M.; Perkins, E.; Howard, J.; Deery, M. J.; Chalker, J. M.; Corzana, F.; Jimenez-Oses, G.; Bernardes, G. J. L. J Am Chem Soc 2018, 140, 4004-4017.PMC5880509
(27) Chemistry for Covalent Modification of Endogenous/Native Proteins: From Test Tubes to Complex Biological Systems. Tamura, T.; Hamachi, I. J Am Chem Soc 2019, 141, 2782-2799
(28) A programmable chemical switch based on triggerable Michael acceptors. Zhuang, J.; Zhao, B.; Meng, X.; Schiffman, J. D.; Perry, S. L.; Vachet, R. W.; Thayumanavan, S. Chem Sci 2020, 11, 2103-2111.PMC8150097
(29) Tunable Methacrylamides for Covalent Ligand Directed Release Chemistry. Reddi, R. N.; Resnick, E.; Rogel, A.; Rao, B. V.; Gabizon, R.; Goldenberg, K.; Gurwicz, N.; Zaidman, D.; Plotnikov, A.; Barr, H.; Shulman, Z.; London, N. J Am Chem Soc 2021, 143, 4979-4992.PMC8041284
(30) Quantitative, Wide-Spectrum Kinase Profiling in Live Cells for Assessing the Effect of Cellular ATP on Target Engagement. Vasta, J. D.; Corona, C. R.; Wilkinson, J.; Zimprich, C. A.; Hartnett, J. R.; Ingold, M. R.; Zimmerman, K.; Machleidt, T.; Kirkland, T. A.; Huwiler, K. G.; Ohana, R. F.; Slater, M.; Otto, P.; Cong, M.; Wells, C. I.; Berger, B. T.; Hanke, T.; Glas, C.; Ding, K.; Drewry, D. H.; Huber, K. V. M.; Willson, T. M.; Knapp, S.; Müller, S.; Meisenheimer, P. L.; Fan, F.; Wood, K. V.; Robers, M. B. Cell Chem Biol 2018, 25, 206-214.e11.PMC5814754
(31) Discovery of the Irreversible Covalent FGFR Inhibitor 8-(3-(4-Acryloylpiperazin-1-yl) propyl)-6-(2,6-dichloro-3,5-dimethoxyphenyl)-2-(methylamino)pyrido[2,3-d]pyrimidin-7(8H)-one (PRN1371) for the Treatment of Solid Tumors. Brameld, K. A.; Owens, T. D.; Verner, E.; Venetsanakos, E.; Bradshaw, J. M.; Phan, V. T.; Tam, D.; Leung, K.; Shu, J.; LaStant, J.; Loughhead, D. G.; Ton, T.; Karr, D. E.; Gerritsen, M. E.; Goldstein, D. M.; Funk, J. O. Journal of Medicinal Chemistry 2017, 60, 6516-6527
(32) Covalent Inhibitors of Interleukin-2 Inducible T Cell Kinase (Itk) with Nanomolar Potency in a Whole-Blood Assay. Zapf, C. W.; Gerstenberger, B. S.; Xing, L.; Limburg, D. C.; Anderson, D. R.; Caspers, N.; Han, S.; Aulabaugh, A.; Kurumbail, R.; Shakya, S.; Li, X.; Spaulding, V.; Czerwinski, R. M.; Seth, N.; Medley, Q. G. Journal of Medicinal Chemistry 2012, 55, 10047-10063
(33) Structural Snapshots from the Oxidative Half-reaction of a Copper Amine Oxidase: IMPLICATIONS FOR O<sub>2</sub>ACTIVATION*. Johnson, B. J.; Yukl, E. T.; Klema, V. J.; Klinman, J. P.; Wilmot, C. M. Journal of Biological Chemistry 2013, 288, 28409-28417
(34) Discovery of (R,E)-N-(7-Chloro-1-(1-[4-(dimethylamino)but-2-enoyl]azepan-3-yl)-1H-benzo[d]imidazol-2-yl)-2-methylisonicotinamide (EGF816), a Novel, Potent, and WT Sparing Covalent Inhibitor of Oncogenic (L858R, ex19del) and Resistant (T790M) EGFR Mutants for the Treatment of EGFR Mutant Non-Small-Cell Lung Cancers. Lelais, G.; Epple, R.; Marsilje, T. H.; Long, Y. O.; McNeill, M.; Chen, B.; Lu, W.; Anumolu, J.; Badiger, S.; Bursulaya, B.; DiDonato, M.; Fong, R.; Juarez, J.; Li, J.; Manuia, M.; Mason, D. E.; Gordon, P.; Groessl, T.; Johnson, K.; Jia, Y.; Kasibhatla, S.; Li, C.; Isbell, J.; Spraggon, G.; Bender, S.; Michellys, P.-Y. Journal of Medicinal Chemistry 2016, 59, 6671-6689
(35) Discovery and characterization of a novel irreversible EGFR mutants selective and potent kinase inhibitor CHMFL-EGFR-26 with a distinct binding mode. Hu, C.; Wang, A.; Wu, H.; Qi, Z.; Li, X.; Yan, X.-E.; Chen, C.; Yu, K.; Zou, F.; Wang, W.; Wang, W.; Wu, J.; Liu, J.; Wang, B.; Wang, L.; Ren, T.; Zhang, S.; Yun, C.-H.; Liu, J.; Liu, Q. Oncotarget 2017, 8
(36) Discovery of a JAK3-Selective Inhibitor: Functional Differentiation of JAK3-Selective Inhibition over pan-JAK or JAK1-Selective Inhibition. Telliez, J.-B.; Dowty, M. E.; Wang, L.; Jussif, J.; Lin, T.; Li, L.; Moy, E.; Balbo, P.; Li, W.; Zhao, Y.; Crouse, K.; Dickinson, C.; Symanowicz, P.; Hegen, M.; Banker, M. E.; Vincent, F.; Unwalla, R.; Liang, S.; Gilbert, A. M.; Brown, M. F.; Hayward, M.; Montgomery, J.; Yang, X.; Bauman, J.; Trujillo, J. I.; Casimiro-Garcia, A.; Vajdos, F. F.; Leung, L.; Geoghegan, K. F.; Quazi, A.; Xuan, D.; Jones, L.; Hett, E.; Wright, K.; Clark, J. D.; Thorarensen, A. ACS Chemical Biology 2016, 11, 3442-3451
(37) Structure-guided development of covalent TAK1 inhibitors. Tan, L.; Gurbani, D.; Weisberg, E. L.; Hunter, J. C.; Li, L.; Jones, D. S.; Ficarro, S. B.; Mowafy, S.; Tam, C.-P.; Rao, S.; Du, G.; Griffin, J. D.; Sorger, P. K.; Marto, J. A.; Westover, K. D.; Gray, N. S. Bioorganic & Medicinal Chemistry 2017, 25, 838-846
(38) Covalent-Allosteric Kinase Inhibitors. Weisner, J.; Gontla, R.; van der Westhuizen, L.; Oeck, S.; Ketzer, J.; Janning, P.; Richters, A.; Mühlenberg, T.; Fang, Z.; Taher, A.; Jendrossek, V.; Pelly, S. C.; Bauer, S.; van Otterlo, W. A. L.; Rauh, D. Angewandte Chemie International Edition 2015, 54, 10313-10316
(39) Discovery of Potent and Selective Covalent Inhibitors of JNK. Zhang, T.; Inesta-Vaquera, F.; Niepel, M.; Zhang, J.; Ficarro, Scott B.; Machleidt, T.; Xie, T.; Marto, Jarrod A.; Kim, N.; Sim, T.; Laughlin, John D.; Park, H.; LoGrasso, Philip V.; Patricelli, M.; Nomanbhoy, Tyzoon K.; Sorger, Peter K.; Alessi, Dario R.; Gray, Nathanael S. Chemistry & Biology 2012, 19, 140-154
(40) Discovery of a Selective Irreversible BMX Inhibitor for Prostate Cancer. Liu, F.; Zhang, X.; Weisberg, E.; Chen, S.; Hur, W.; Wu, H.; Zhao, Z.; Wang, W.; Mao, M.; Cai, C.; Simon, N. I.; Sanda, T.; Wang, J.; Look, A. T.; Griffin, J. D.; Balk, S. P.; Liu, Q.; Gray, N. S. ACS Chemical Biology 2013, 8, 1423-1428
(41) Targeting transcription regulation in cancer with a covalent CDK7 inhibitor. Kwiatkowski, N.; Zhang, T.; Rahl, P. B.; Abraham, B. J.; Reddy, J.; Ficarro, S. B.; Dastur, A.; Amzallag, A.; Ramaswamy, S.; Tesar, B.; Jenkins, C. E.; Hannett, N. M.; McMillin, D.; Sanda, T.; Sim, T.; Kim, N. D.; Look, T.; Mitsiades, C. S.; Weng, A. P.; Brown, J. R.; Benes, C. H.; Marto, J. A.; Young, R. A.; Gray, N. S. Nature 2014, 511, 616-620
(42) Covalent targeting of remote cysteine residues to develop CDK12 and CDK13 inhibitors. Zhang, T.; Kwiatkowski, N.; Olson, C. M.; Dixon-Clarke, S. E.; Abraham, B. J.; Greifenberg, A. K.; Ficarro, S. B.; Elkins, J. M.; Liang, Y.; Hannett, N. M.; Manz, T.; Hao, M.; Bartkowiak, B.; Greenleaf, A. L.; Marto, J. A.; Geyer, M.; Bullock, A. N.; Young, R. A.; Gray, N. S. Nature Chemical Biology 2016, 12, 876-884
(43) A comprehensive mathematical model for three-body binding equilibria. Douglass, E. F., Jr.; Miller, C. J.; Sparer, G.; Shapiro, H.; Spiegel, D. A. J Am Chem Soc 2013, 135, 6092-9.PMC3717292
(44) High-throughput phosphoproteomics reveals in vivo insulin signaling dynamics. Humphrey, S. J.; Azimifar, S. B.; Mann, M. Nature Biotechnology 2015, 33, 990-U142
(45) Quantitative-Proteomic Comparison of Alpha and Beta Cells to Uncover Novel Targets for Lineage Reprogramming. Choudhary, A.; Hu He, K.; Mertins, P.; Udeshi, N. D.; Dančík, V.; Fomina-Yadlin, D.; Kubicek, S.; Clemons, P. A.; Schreiber, S. L.; Carr, S. A.; Wagner, B. K. PLOS ONE 2014, 9, e95194
(46) MHC class I-associated phosphopeptides are the targets of memory-like immunity in leukemia. Cobbold, M.; De La Pena, H.; Norris, A.; Polefrone, J. M.; Qian, J.; English, A. M.; Cummings, K. L.; Penny, S.; Turner, J. E.; Cottine, J.; Abelin, J. G.; Malaker, S. A.; Zarling, A. L.; Huang, H. W.; Goodyear, O.; Freeman, S. D.; Shabanowitz, J.; Pratt, G.; Craddock, C.; Williams, M. E.; Hunt, D. F.; Engelhard, V. H. Sci Transl Med 2013, 5, 203ra125.PMC4071620
(47) Complementary IMAC enrichment methods for HLA-associated phosphopeptide identification by mass spectrometry. Abelin, J. G.; Trantham, P. D.; Penny, S. A.; Patterson, A. M.; Ward, S. T.; Hildebrand, W. H.; Cobbold, M.; Bai, D. L.; Shabanowitz, J.; Hunt, D. F. Nat Protoc 2015, 10, 1308-18.PMC4640213
(48) Rapid labelling and covalent inhibition of intracellular native proteins using ligand-directed N-acyl-N-alkyl sulfonamide. Tamura, T.; Ueda, T.; Goto, T.; Tsukidate, T.; Shapira, Y.; Nishikawa, Y.; Fujisawa, A.; Hamachi, I. Nat Commun 2018, 9, 1870.PMC5951806
(49) Enhanced Suppression of a Protein-Protein Interaction in Cells Using Small-Molecule Covalent Inhibitors Based on an N-Acyl-N-alkyl Sulfonamide Warhead. Ueda, T.; Tamura, T.; Kawano, M.; Shiono, K.; Hobor, F.; Wilson, A. J.; Hamachi, I. J Am Chem Soc 2021, 143, 4766-4774
(50) Activation Method to Prepare a Highly Reactive Acylsulfonamide “Safety-Catch” Linker for Solid-Phase Synthesis1. Backes, B. J.; Virgilio, A. A.; Ellman, J. A. Journal of the American Chemical Society 1996, 118, 3055-3056
(51) Discovery of a Type III Inhibitor of LIM Kinase 2 That Binds in a DFG-Out Conformation. Goodwin, N. C.; Cianchetta, G.; Burgoon, H. A.; Healy, J.; Mabon, R.; Strobel, E. D.; Allen, J.; Wang, S.; Hamman, B. D.; Rawlins, D. B. ACS Med Chem Lett 2015, 6, 53-7.PMC4291701
(52) Structural basis for the action of the drug trametinib at KSR-bound MEK. Khan, Z. M.; Real, A. M.; Marsiglia, W. M.; Chow, A.; Duffy, M. E.; Yerabolu, J. R.; Scopton, A. P.; Dar, A. C. Nature 2020, 588, 509-514.PMC7746607
(53) Discovery of Small Molecule RIP1 Kinase Inhibitors for the Treatment of Pathologies Associated with Necroptosis. Harris, P. A.; Bandyopadhyay, D.; Berger, S. B.; Campobasso, N.; Capriotti, C. A.; Cox, J. A.; Dare, L.; Finger, J. N.; Hoffman, S. J.; Kahler, K. M.; Lehr, R.; Lich, J. D.; Nagilla, R.; Nolte, R. T.; Ouellette, M. T.; Pao, C. S.; Schaeffer, M. C.; Smallwood, A.; Sun, H. H.; Swift, B. A.; Totoritis, R. D.; Ward, P.; Marquis, R. W.; Bertin, J.; Gough, P. J. ACS Med Chem Lett 2013, 4, 1238-43.PMC4027519
(54) Single and Dual Targeting of Mutant EGFR with an Allosteric Inhibitor. To, C.; Jang, J.; Chen, T.; Park, E.; Mushajiang, M.; De Clercq, D. J. H.; Xu, M.; Wang, S.; Cameron, M. D.; Heppner, D. E.; Shin, B. H.; Gero, T. W.; Yang, A.; Dahlberg, S. E.; Wong, K. K.; Eck, M. J.; Gray, N. S.; Janne, P. A. Cancer Discov 2019, 9, 926-943.PMC6664433
(55) Unfolded Protein Response in Cancer: IRE1alpha Inhibition by Selective Kinase Ligands Does Not Impair Tumor Cell Viability. Harrington, P. E.; Biswas, K.; Malwitz, D.; Tasker, A. S.; Mohr, C.; Andrews, K. L.; Dellamaggiore, K.; Kendall, R.; Beckmann, H.; Jaeckel, P.; Materna-Reichelt, S.; Allen, J. R.; Lipford, J. R. ACS Med Chem Lett 2015, 6, 68-72.PMC4291719
(56) A novel approach to the discovery of small-molecule ligands of CDK2. Martin, M. P.; Alam, R.; Betzi, S.; Ingles, D. J.; Zhu, J. Y.; Schonbrunn, E. Chembiochem 2012, 13, 2128-36.PMC3483082
(57) Compound Selectivity and Target Residence Time of Kinase Inhibitors Studied with Surface Plasmon Resonance. Willemsen-Seegers, N.; Uitdehaag, J. C. M.; Prinsen, M. B. W.; de Vetter, J. R. F.; de Man, J.; Sawa, M.; Kawase, Y.; Buijsman, R. C.; Zaman, G. J. R. J Mol Biol 2017, 429, 574-586
(58) ABC transporters in cancer: more than just drug efflux pumps. Fletcher, J. I.; Haber, M.; Henderson, M. J.; Norris, M. D. Nat Rev Cancer 2010, 10, 147-56
(59) A Call for Systematic Research on Solute Carriers. Cesar-Razquin, A.; Snijder, B.; Frappier-Brinton, T.; Isserlin, R.; Gyimesi, G.; Bai, X.; Reithmeier, R. A.; Hepworth, D.; Hediger, M. A.; Edwards, A. M.; Superti-Furga, G. Cell 2015, 162, 478-87
(60) A widespread role for SLC transmembrane transporters in resistance to cytotoxic drugs. Girardi, E.; César-Razquin, A.; Lindinger, S.; Papakostas, K.; Konecka, J.; Hemmerich, J.; Kickinger, S.; Kartnig, F.; Gürtl, B.; Klavins, K.; Sedlyarov, V.; Ingles-Prieto, A.; Fiume, G.; Koren, A.; Lardeau, C.-H.; Kumaran Kandasamy, R.; Kubicek, S.; Ecker, G. F.; Superti-Furga, G. Nature Chemical Biology 2020, 16, 469-478
(61) Protein Degradation by In-Cell Self-Assembly of Proteolysis Targeting Chimeras. Lebraud, H.; Wright, D. J.; Johnson, C. N.; Heightman, T. D. ACS Central Science 2016, 2, 927-934.

### Example 3—Proximity-Inducing Molecules with Cleavable Linker for Extracellular and Intracellular Targets

### Introduction

Bifunctional molecules have emerged as powerful and novel therapeutic modalities due to their ability to enhance existing protein-protein interactions (PPIs) or promote neo-PPIs. One particularly interesting category of this class are bifunctional molecules which induce post translational modifications on a protein of interest (POI). Current design of such bifunctional molecules consists of a three-component “dumbbell-like” structure. The two protein binders are connected via a linker of appropriate length (FIG. 91A). This design has been successfully applied for PTM induction using proteolysis targeting chimeras (PROTACs)¹, phosphorylation inducing chimeras (PHICS)², phosphatase recruitment chimeras (PHORCs)³, deubiquitinase-targeting chimeras (DUBTACs)⁴ etc. Biological evaluation of these bifunctional molecules reveals that perturbation of the PTM homeostasis results in phenotypic alterations of therapeutic relevancy. This is possible because the bifunctional molecule “forces” increased effective molarity between the POI and the PTM writer/eraser. For example, PROTACs which are the most widely studied group of bifunctional molecules, increase ubiquitinalation of the targeted protein leading to its degradation via the proteasome pathway, while DUBTACs have the opposite effect. On the other hand, PHICS induce phosphorylation on target protein altering its charge state, with detrimental effects on protein structure and function. Interestingly the corresponding eraser PHORCs can induce similar affects.

It is apparent from the current design of such molecules that on the site of the PTM writer/eraser the use of high-affinity, non-inhibitory and allosteric binders is required (FIG. 91B). Among the known binders few molecules have all desired characteristics limiting the available chemical matter can be utilized as well as the PTM writer/erasers can be targeted. In addition, current dumbbell design paradigm uses non-covalent molecules which reduces its applications. Covalency can increase binding potency, prolong ternary complex formation, and expand the applications of bifunctional molecules. Recently, generation of MHC-restricted neoantigens utilizing covalent chemistry has gained increased interest⁵, with application of bifunctional molecules on such systems yet to be explored.

Applicants' laboratory has developed a new design paradigm which bypasses the limitations of current bifunctional design (FIG. 91C). It utilizes thiol mediated group-transfer chemistry for the generation of bifunctional molecules which will form ternary complexes in a two-step pathway. Initially, a covalent binder occupies the active site of the PTM writer/eraser facilitating Michael addition, followed by a retro-aza-Michael which releases the binder (this chemistry was originally reported by AstraZeneca⁶ (in 2013) without further investigation). The released non-covalent compound typically has reduced affinity, which in combination with the high abundance of the natural substrate results in its displacement from the active site permitting PTM induction/removal on the target protein. This technology is complementary to the current bifunctional design because it allows the utilization of allosteric or active site inhibitors, which represent most chemical matter on the PTM space. In addition, the technology, allows for the generation of bifunctional molecules with extracellular applications.

### Results and Discussion

### Design and Synthesis of FGFR-Recruiting Chimeras.

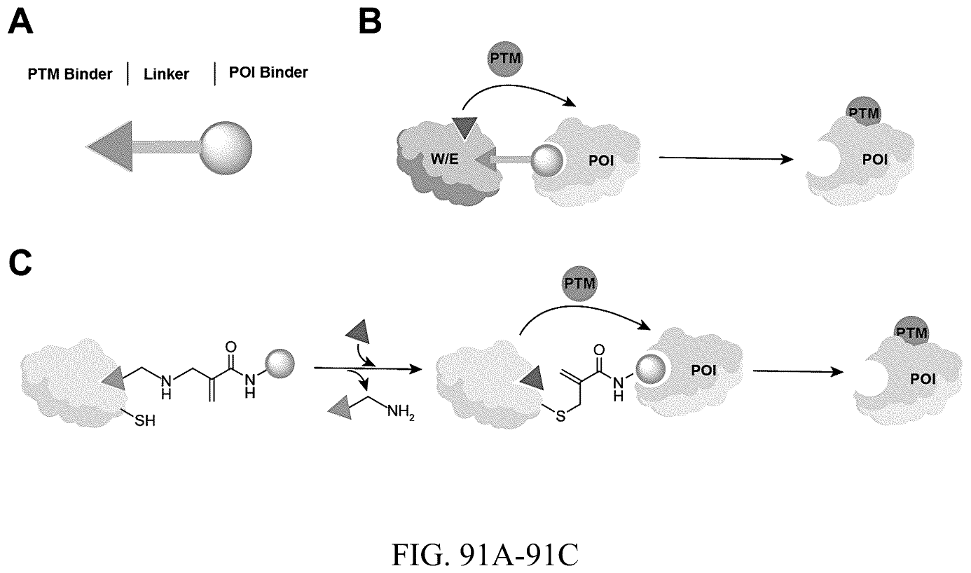

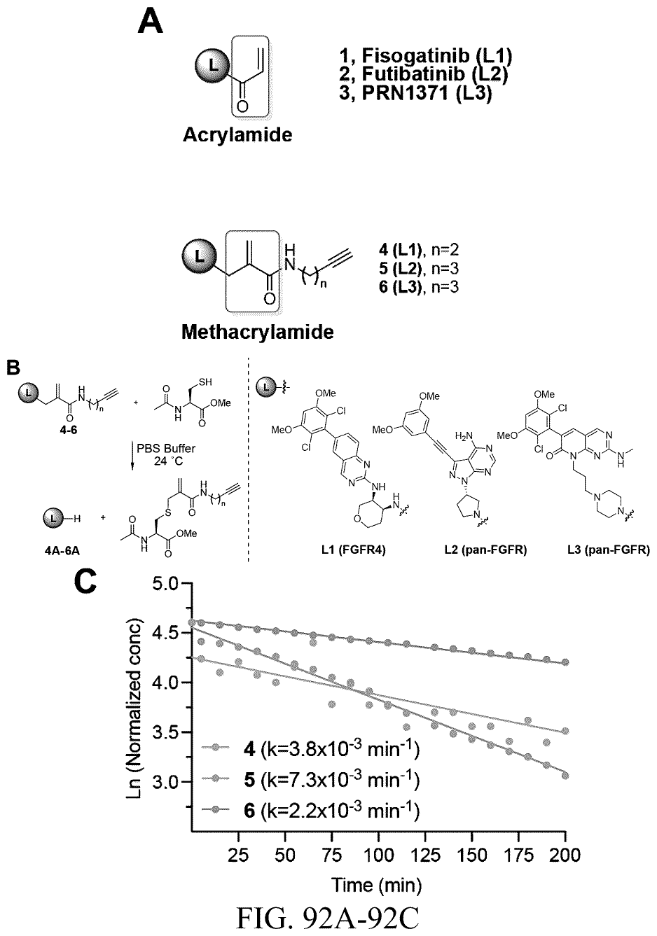

Given the key role played by Fibroblast growth factor receptor (FGFR) in cellular signaling for cell growth, proliferation and, division and, widespread occurrence of aberrant FGFR signaling in different type of cancers involving different isoforms (namely FGFR1-4), Applicants chose to three FGFR covalent inhibitors (1, 2, 3) namely Fisogatinib (FGFR4), Futibatinib and PRN1317 (pan-FGFR) and synthesized the corresponding methacrylamide compounds containing alkyne handle (4, 5, 6) (FIG. 92A). To validate the proposed addition elimination reactivity of the methacrylamide warhead with cysteine, Applicants studied the reactivity of the compounds 4, 5 and 6 with N-Acetyl-cysteine methyl ester at room temperature in PBS buffer (pH 7.4) in a time dependent manner (FIG. 92B). Applicants observed the decay of alkyne with subsequent rise in the concentration of the corresponding non-covalent kinase binder (eliminated amine) (FIG. 92C).

### Demonstration of Phosphorylation on Proof-of-Concept Target BRD4.

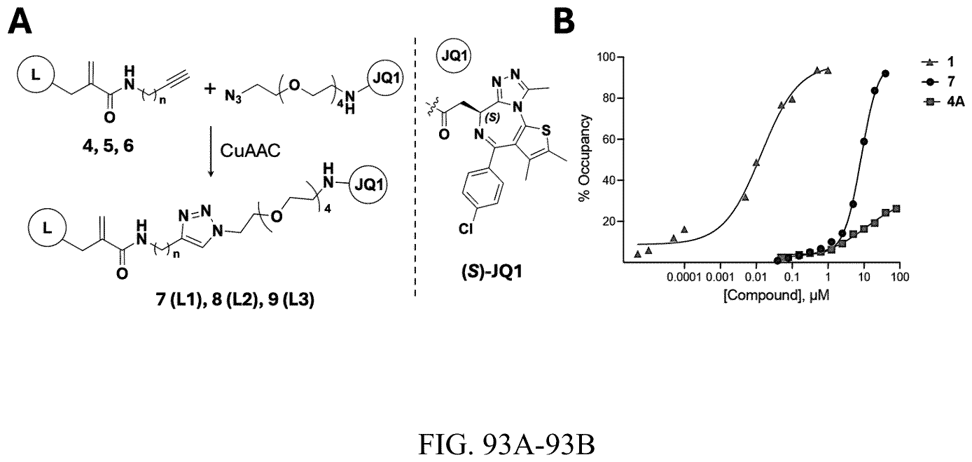

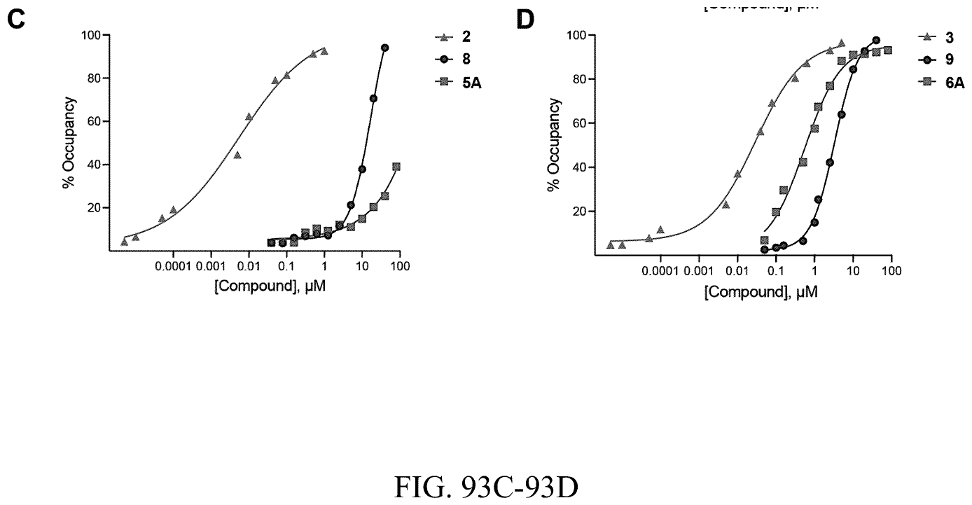

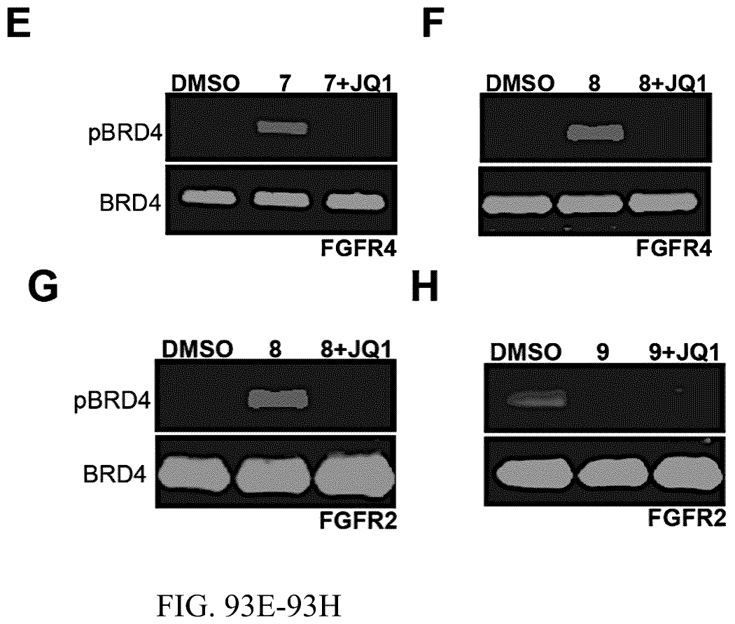

All four isoforms of FGFRs have conserved kinase domains with 74-92% sequence identity. Several covalent inhibitors were developed to target cysteines in the FGFRs, most of them are Pan-FGFR inhibitors targeting P-loop cysteine of the kinase domain and some exploit the presence of unique Cysteine residue in the hinge region of FGFR4 kinase. Three covalent inhibitors in the clinical trials were selected for the designing of PHICS molecules: FGFR4 selective Fisogatinib, Pan-FGFR inhibitors Futibatinib and PRN1317. The inhibitory potential of these molecules is dictated by their covalency and permanent occupancy of the ATP binding pocket. Applicants have used group transfer chemistry to label the protein with the target binder. For designing the PHICS with cysteine group transfer chemistry (CGT-PHICS), inhibitor scaffolds were exploited for their selectivity to the FGFR kinases and group transfer chemistry to label specific cysteine with the target protein binder leaving behind the ineffective non-covalent inhibitor in the ATP binding pocket of the kinase. BRD4 binder(S)-JQ1 was conjugated with the alkyne compounds by copper catalyzed azide-alkyne click reaction (CuAAC) to generate CGT-PHICS 7, 8 and 9 (FIG. 93A). Cellular labeling of FGFR4 protein was confirmed by the In-gel fluorescence and Intact-mass spectrometry (Supp. Figures X and Y). Nano-BRET assay was performed to determine the occupancy of the released non-covalent molecules in the ATP binding pocket. Nano-BRET assay measures the signal for the binding of fluorophore conjugated tracer molecule to the ATP binding pocket of the kinase, which fluoresces upon excitation from the luminescence signal from the FGFR-nanoluc. In the nano-BRET assay, HEK293T cells were transfected with FGFR4-nanoluc and treated with the covalent (1, 2 and 3), non-Covalent (4A, 5A and 6A) and CGT-PHICS (7, 8 and 9). A massive drop in the occupancy was observed in the case of the CGT-PHICS (7, 8 and 9) as compared to the covalent inhibitors (1, 2 and 3) (FIGS. 93B, 93C and 93D). The FGFR4 pocket was fully occupied at very low concentration by the covalent (1, 2 and 3) molecules, whereas lower occupancy by was observed for the CGT-PHICS (7, 8 and 9) even at very high concentrations (FIGS. 93B, 93C and 93D). Interestingly, the non-covalent molecule 6A showed higher occupancy as compared to its cognate PHICS 9. These observations point toward the fact that CGT-PHICS (7, 8 and 9) upon labeling do not effectively occupy the ATP pocket after the release of non-covalent inhibitor, hence could lose its potency to inhibit the kinase. To test the hypothesis, PHICS molecules designed by conjugating the FGFR inhibitors with JQ1 were used to target BRD4 proteins for phosphorylation. The phosphorylation on BRD4 was observed in the presence of CGT-PHICS (7 and 8) using FGFR4 (FIGS. 93E and 93F) and with its variant FGFR2 using CGT-PHICS 8 (FIG. 93G), whereas CGT-PHICS 9 doesn't induce any phosphorylation on BRD4 in HEK293T cells (FIG. 93H). The FGFR phosphorylations on BRD4 in the presence of PHICS also corroborated with the occupancy data and rate constant, where CGT-PHICS (7 and 8) and methacrylamide (4 and 5) showed lower occupancy and higher rate constant as compared with CGT-PHICS 9 and 6, respectively (FIGS. 92C, 93B, 93C and 93D). Overall, PHICS based group transfers on FGFRs suggest that FGFRs can tolerate huge modifications on the hinge Cys-552 (FGFR4) or the P-loop Cys-447 (FGFR4 and FGFR2) and the released non-covalent inhibitor in the ATP binding pocket will be replaced by the presence of a high concentration of ATP in the cell keeping the kinase active for target protein phosphorylation.

### Functional Activity of FGFR-Recruiting Chimeras.

RTKs regulate the proliferation, differentiation, and metabolism of cells by inducing downstream signaling in response to external factors such as Fibroblast Growth Factors (FGFs). FGFs binds extracellular domains of FGFRs and induce the dimerization resulting in cross-phosphorylation and downstream signaling. Chemical inducer of dimerization (CID) molecule FK1012 (a dimeric FKPB binder) was used intracellularly for the dimerization of RTKs fused with FKBP to induce signaling.

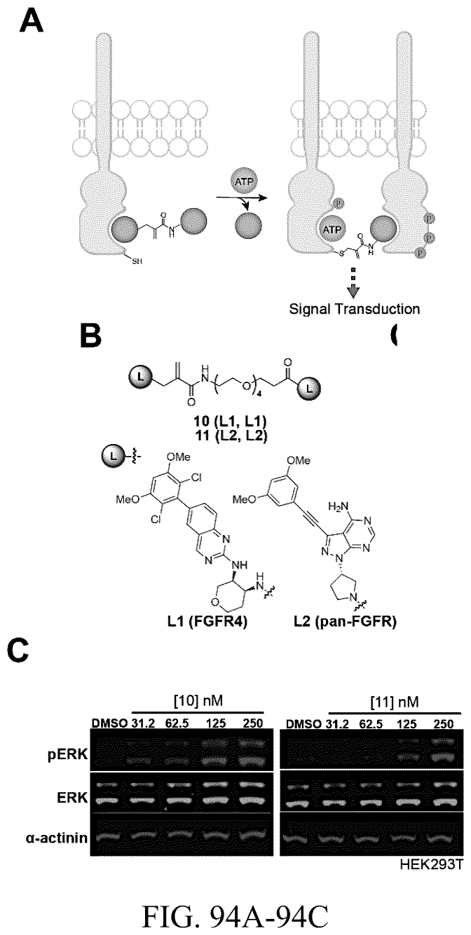

However, these systems required artificial integration of the FKBP gene on the target gene for spatial-temporal control. Since the group transfer chemistry on several kinases were successfully performed without affecting their activity. Applicants have designed dimeric PHICS (dPHICS) molecules for inducing dimerization and phosphorylation of FGFR kinases subsequently leading to the induction of downstream pathways (FIG. 94A). Among several advantages, as compared to previous techniques (FKBP), genetic modification of the target gene was not required. In a paradox, inhibitors were used for designing PHICS and downstream signaling. FGFR signaling complex may lead to the activation of four signaling branches PI3K/AKT, PLCγ/DAG/PKC, RAS/RAF/MAPK, and GSK3β/β-catenin, which are involved in proliferation, antiapoptotic, angiogenesis and drug resistance. dPHICS equipped with group transfer chemistry were synthesized by joining either two FGFR4 selective Fisogatinib or Pan-FGFR inhibitor Futibatinib (FIG. 94B). HEK293T cells were treated with the PHICS 10 and PHICS 12 molecules for 90 minutes and the cell lysate was probed for ERK phosphorylation. A dose dependent increase in the pERK was observed for both PHICS 11 and 12, which gives a maximum pERK signal at 250 nM and turns inhibitory at 500 nM (FIG. 94C). This suggests that dPHICS can be used for the tunable intracellular activation of the FGFR downstream signaling.

### Generalization to Other Kinases.

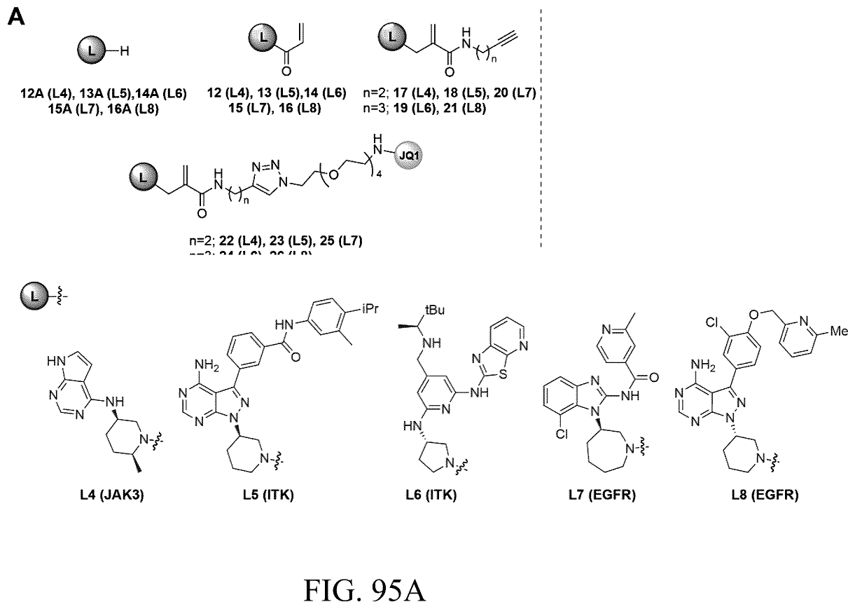

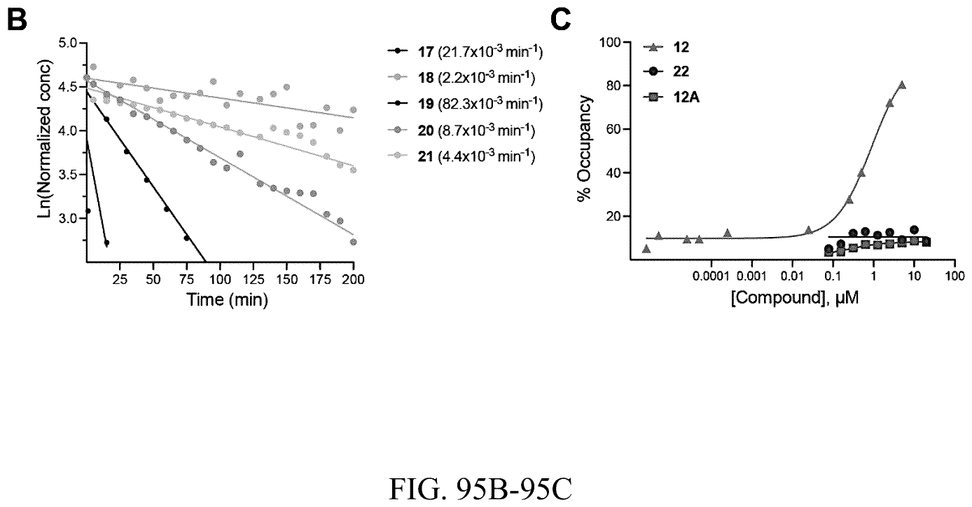

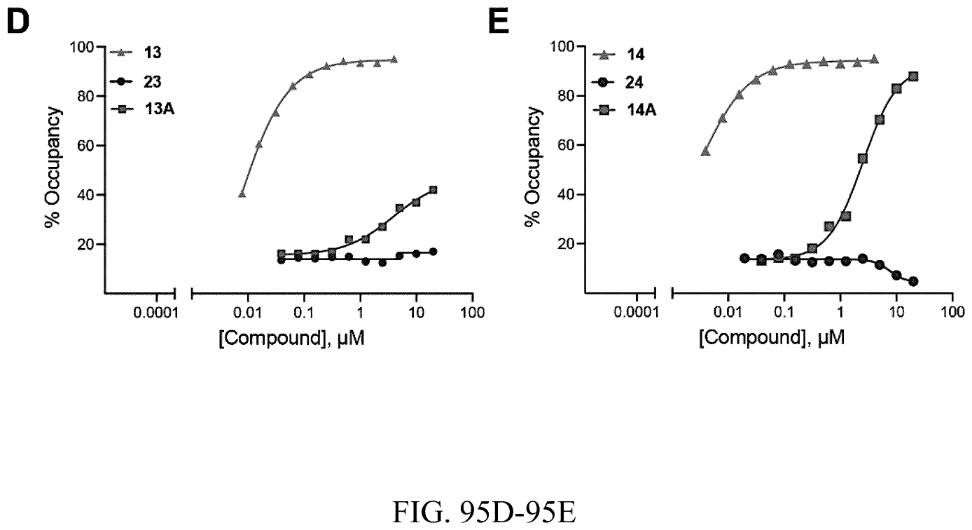

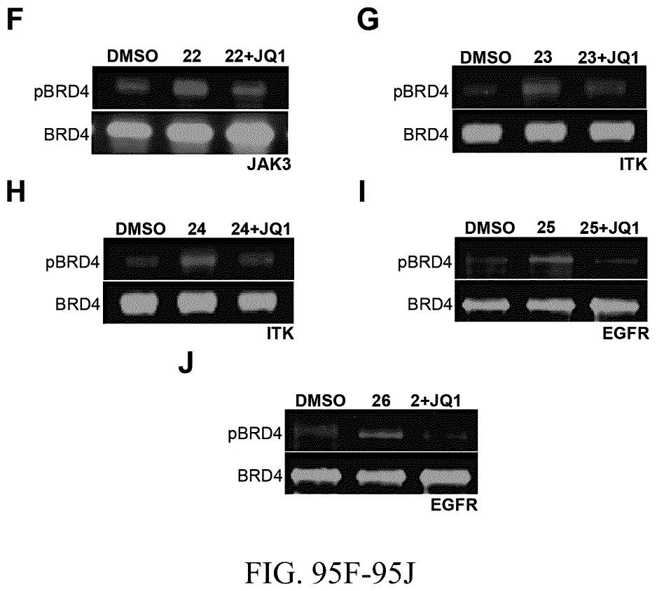

Having established the cysteine group transfer chemistry with FGFR, Applicants wanted to check the generality of group transfer by targeting cysteine of other kinases. Applicants chose covalent kinase inhibitors of Janus kinase 3 (JAK3) (12), IL2 inducible T cell kinase (ITK) (18 and 19) and Epidermal growth factor receptor (EGFR) (20 and 21) as the target and prepared corresponding methacrylamide compounds containing alkyne handle (17-21) and (S)-JQ1 clicked PHICS (22-26) (FIG. 95A). Similar to the FGFR compounds, the methacrylamide compounds (17-21) showed similar reactivity with N-Acetyl-cysteine methyl ester (FIG. 95B). A nano-BRET assay was performed on JAK3 and ITK to determine the occupancy of covalent (12, 13 and 14), non-Covalent (12A, 13A and 14A) and CGT-PHICS (22, 23 and 24). CGT-PHICS showed the lowest occupancy of the ATP binding pockets of the JAK3 and ITK as compared with the most obvious covalent and even non-covalent inhibitors (FIGS. 95C, 95D and 95E). All the(S)-JQ1 clicked PHICS were active in inducing significant levels of BRD4 phosphorylation as compared to DMSO control. Competition with (+) JQ1 at a 1:4 ratio of the PHICS reduced the BRD4 phosphorylation, confirming CGT-PHICS-dependent phosphorylations on BRD4 (FIGS. 95F, 95G, 95H, 95I and 95J).

### Example 4

Post-translational modifications (PTMs) on proteins play a vital role in an organism and have been implicated in several fundamental mechanistic questions surrounding insulin and insulin-producing β cells. For instance, in type 1 diabetes, B-cells are considered foreign by the immune system, triggering their destruction. However, as with other autoimmune disorders, the molecules responsible for the “foreignness” are unknown, though PTMs have been implicated as potential culprits. Furthermore, despite being discovered a century ago, the mechanisms of action of both insulin and insulin resistance remain unclear, with recent advances pointing fingers at PTMs as unexpected drivers of information flow for not just insulin but other signaling molecules.

While editing modifications on DNA/RNA have become facile, thanks to CRISPR-editors, such editors are scarce for PTMs. As nature has evolved many enzymes that “write” or “erase” PTMs, Applicants propose using this to Applicants advantage by building a platform of PTM editors that will recruit endogenous cellular enzymes to append or remove PTMs on a protein-of-interest. Applicants have successfully developed such editors for an abundant PTM, phosphorylation, and demonstrated the utility of such editors to recapitulate known biology and uncover new cellular functions. Applicants will now build a general platform for PTM editing with the long-term goal of making it as facile as CRISPR-editors. Applicants will thus apply the PTM editors to understand the molecular basis of autoimmunity and the molecular mechanism of insulin and insulin resistance. Furthermore, Applicants will use these PTM editors to append identified immunogenic PTMs to “undruggable” oncogenes to mark such cells for destruction and will explore PTM editors as a low-cost, injection-free alternative to insulin and insulin resistance. Overall, Applicants aspire to develop this broadly applicable editing technology to understand fundamental biological questions with consequences to human health.

### Motivation

CRISPR-based technologies are revolutionizing basic science and biomedicine by making modifications of DNA/RNA facile.1 However, technologies to systematically “write” or “erase” posttranslational modifications (PTMs) on proteins, which are vital in diverse cellular processes, are non-existent.2 The long-term goal is to develop a suite of CRISPR-equivalent platform technologies for robust PTM editing. While such a platform, like CRISPR-based technologies, will have many applications, Applicants will apply it in two areas. First, Applicants will delineate the fundamental roles of PTMs in triggering autoimmunity and apply that understanding to impart “immunogenicity” to undruggable proteins in cancer. Second, Applicants will delineate the roles of PTMs in the condensate biology of insulin signaling and resistance and apply that understanding to engineer a low-cost injection-free alternative to insulin.

### How to Design PTM-Writing Chimeras (PWCs)?

Nature employs PTMs for rapid information transfer (e.g., receptor signaling) as PTM induction or removal is highly dynamic, often operating on time scales of minutes to seconds. Furthermore, PTMs occur at low stoichiometry (dose controlled) and are rapidly reversible.3 Inspired by cell biologists who use small molecules to delineate highly dynamic cellular processes4,5 and chemists who use proximity-induction to alter enzyme specificity, 6 Applicants will develop small-molecule-based technologies that “write” or “erase” PTMs on target proteins by forcing proximity between the PTM-modulating enzyme and the target.

Conventional proximity-inducing bifunctional molecules, wherein one end binds to a PTM-modulating enzyme and the other end binds to a target protein, can induce or erase PTMs.7,8 However, the enzyme-binding end requires high-affinity and non-inhibitory binders that are scarcely available. Because inhibitors of nearly every PTM-modulating enzyme are available (or easily designed/discovered), Applicants instead use inhibitors to covalently transfer the target-protein binder onto the enzyme at low stoichiometry. Such transfer will induce proximity between the enzyme and target protein, leading to PTM editing.

### Experimental Validation of Kinase PWCs.

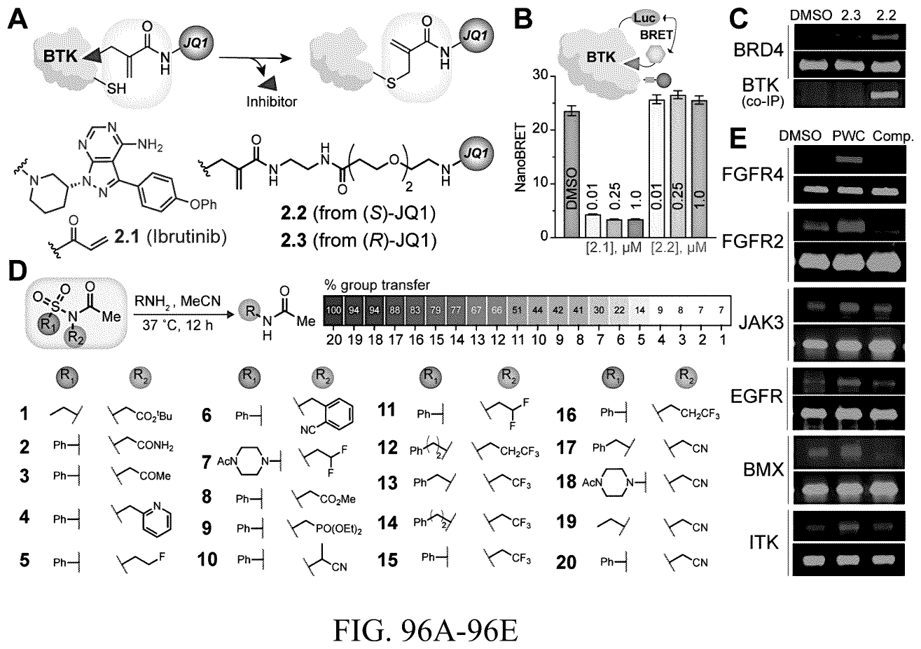

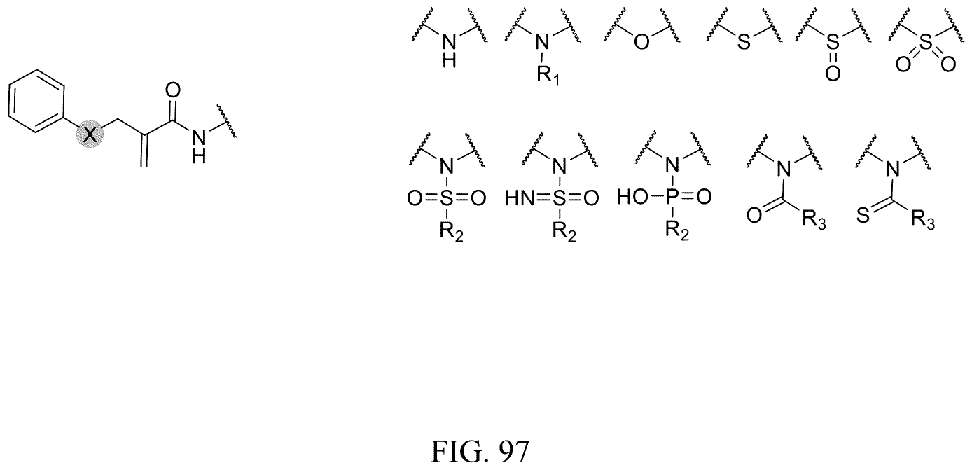

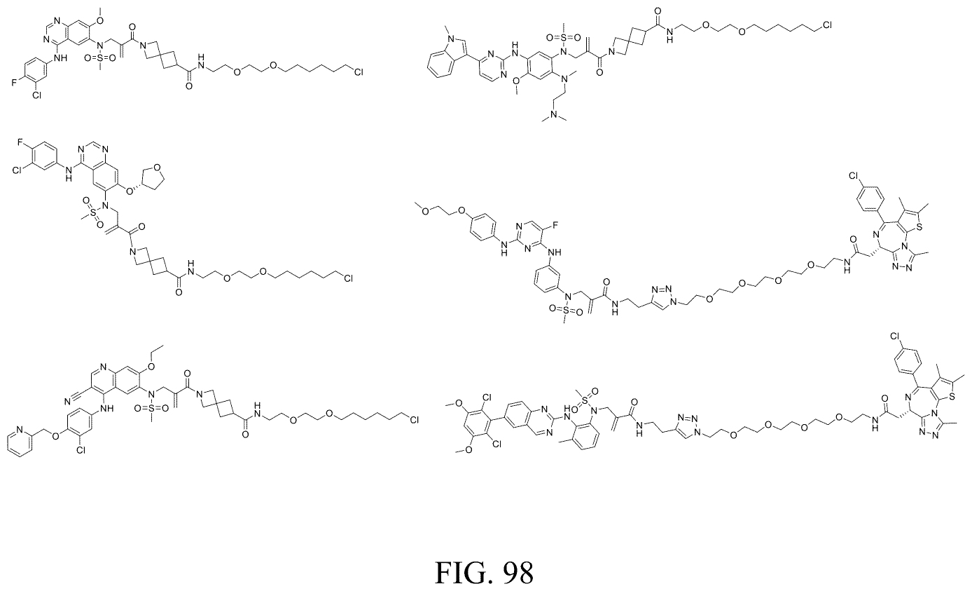

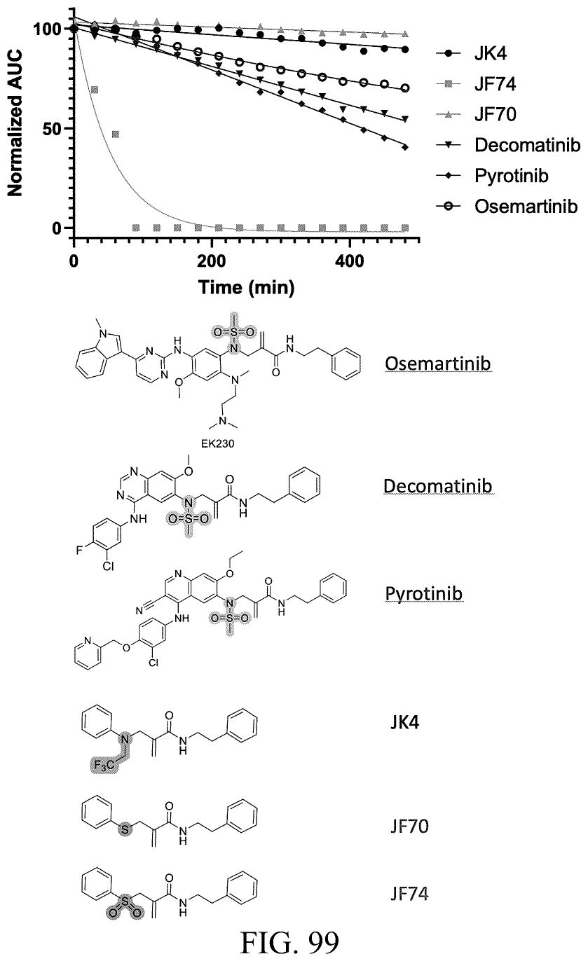

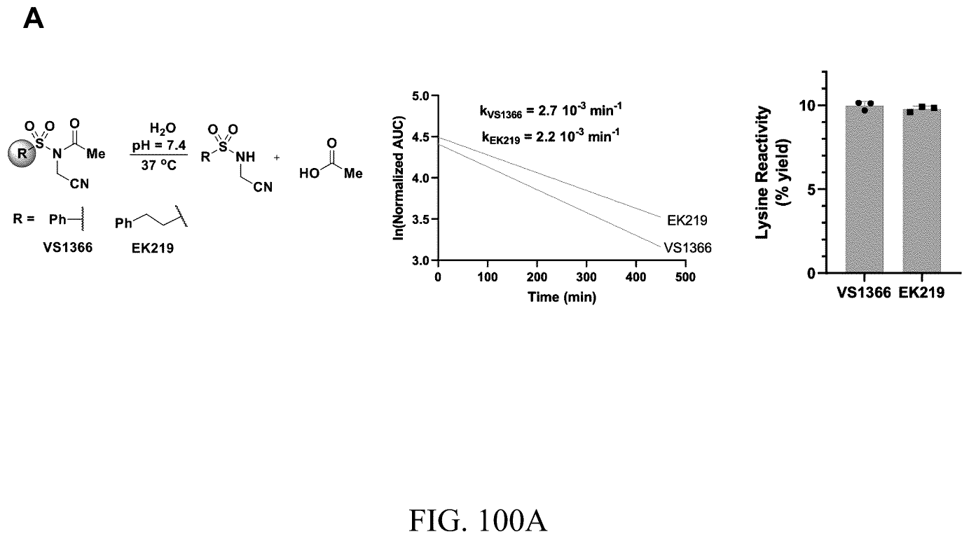

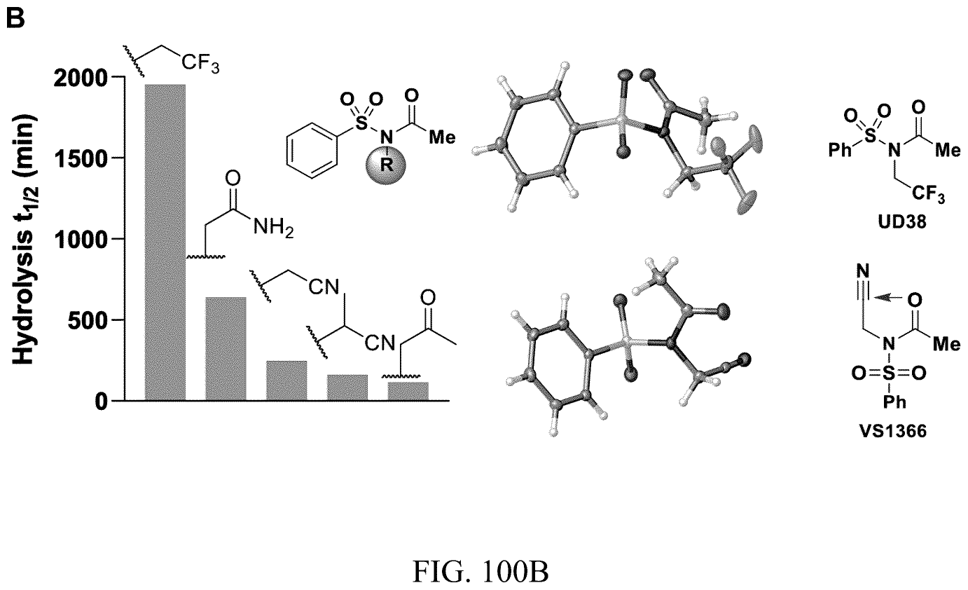

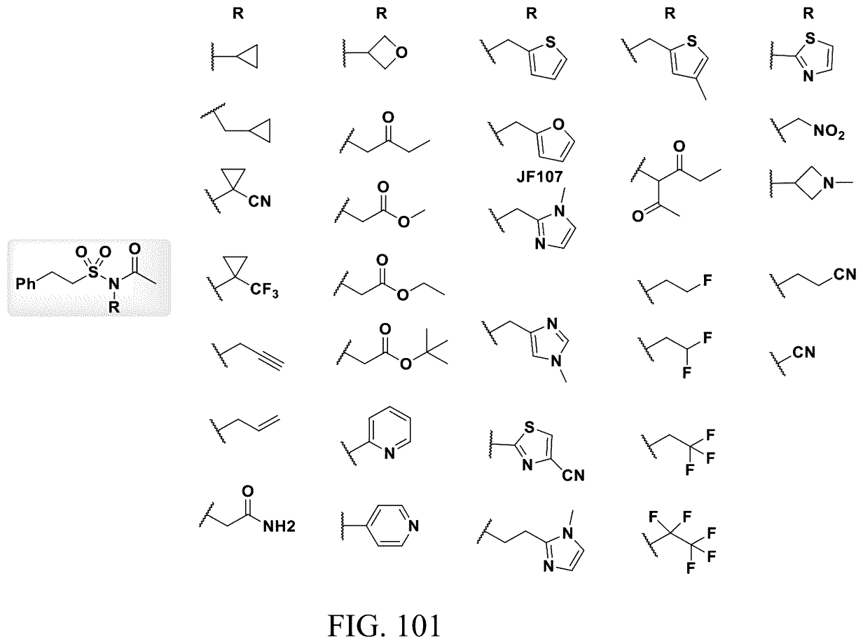

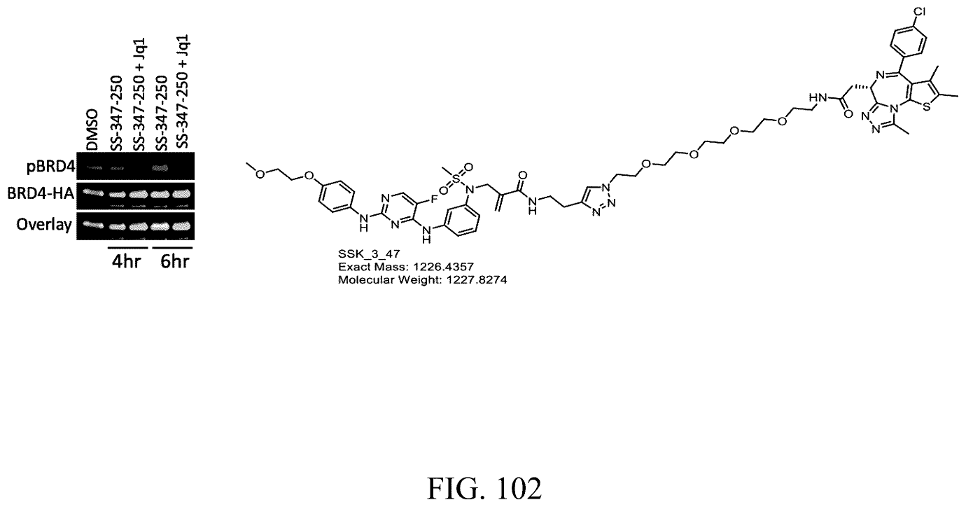

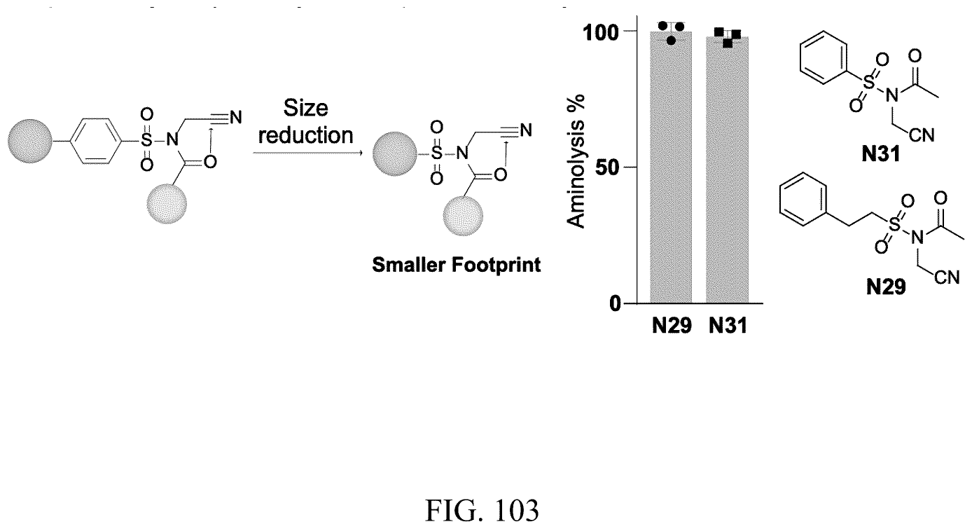

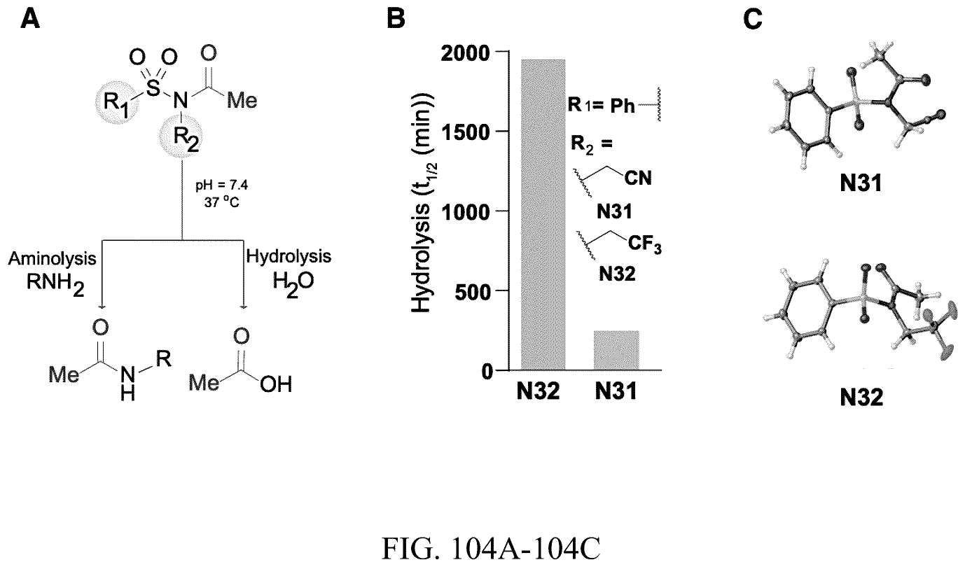

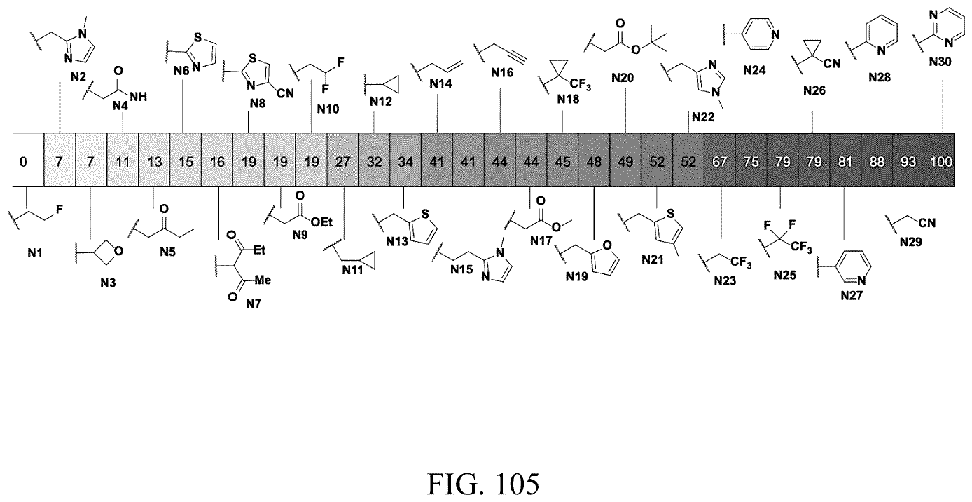

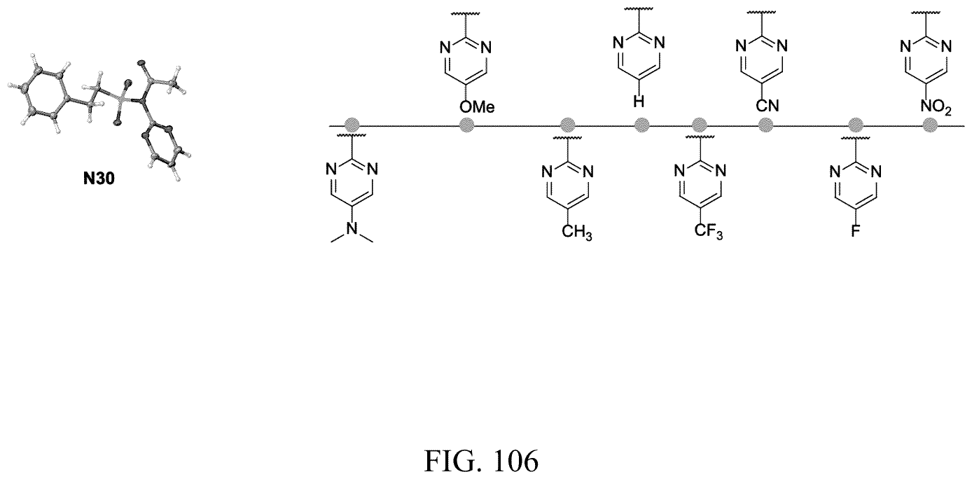

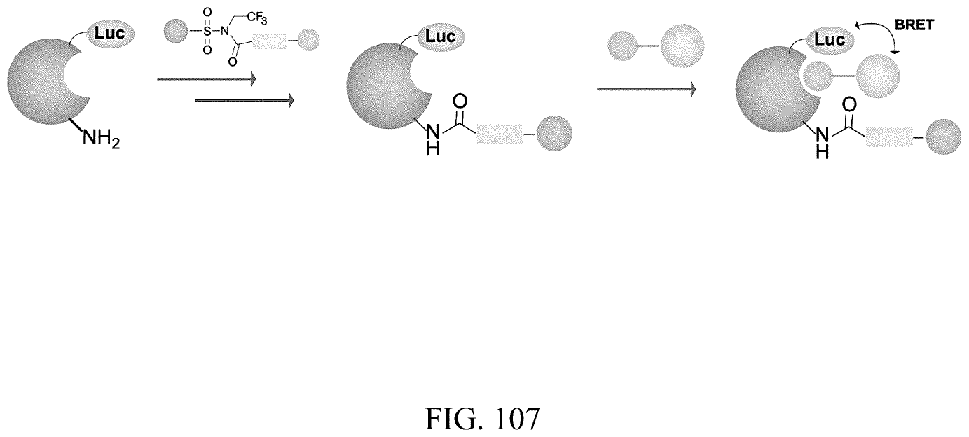

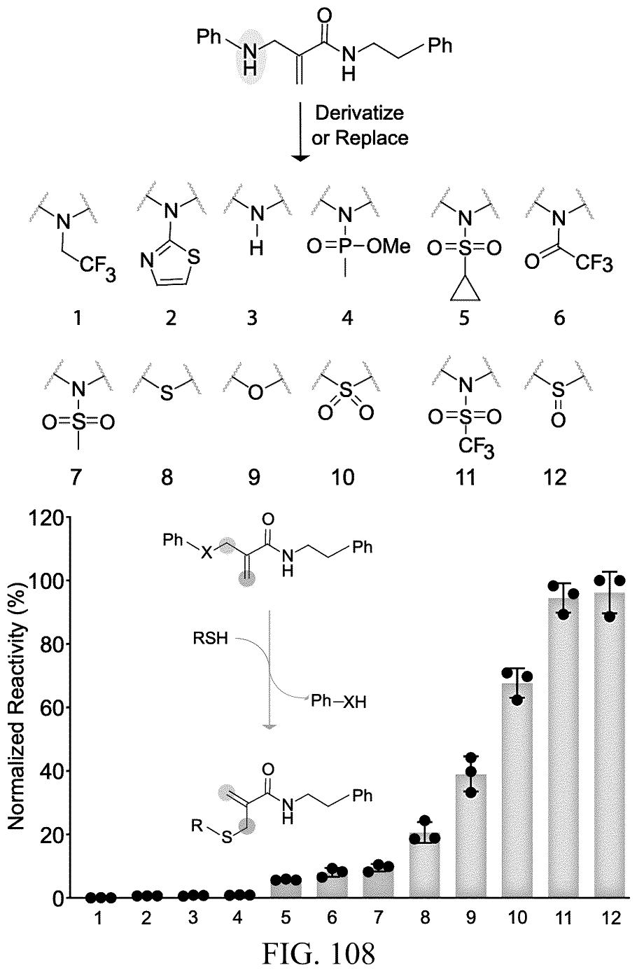

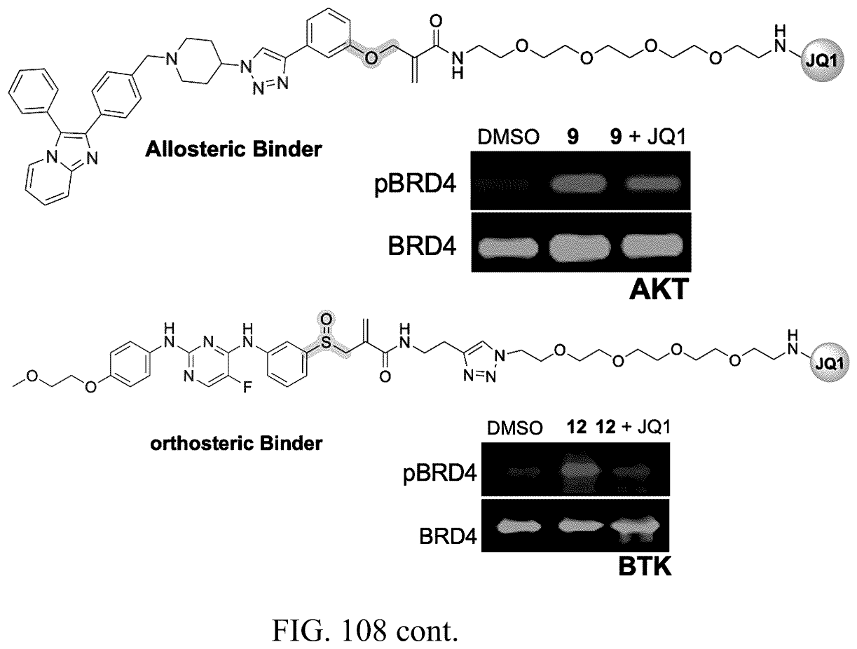

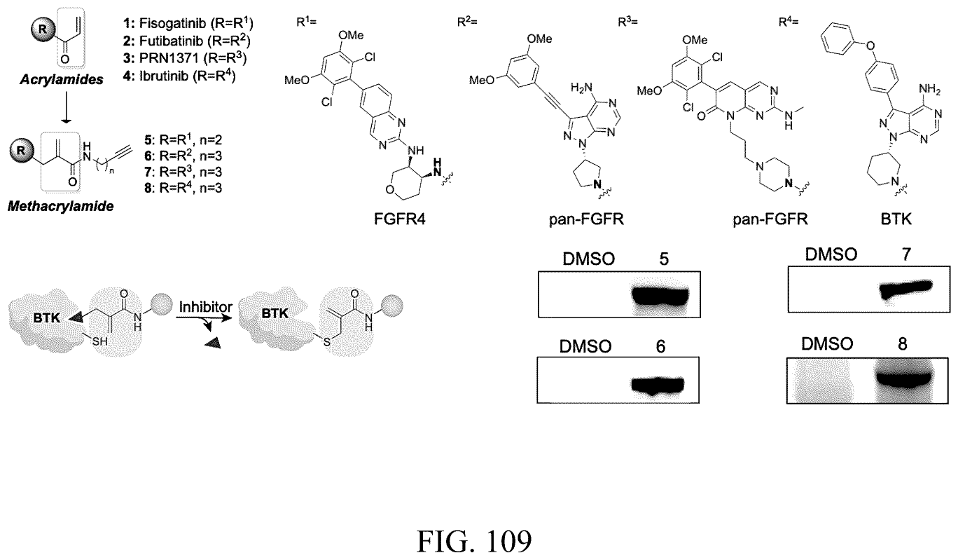

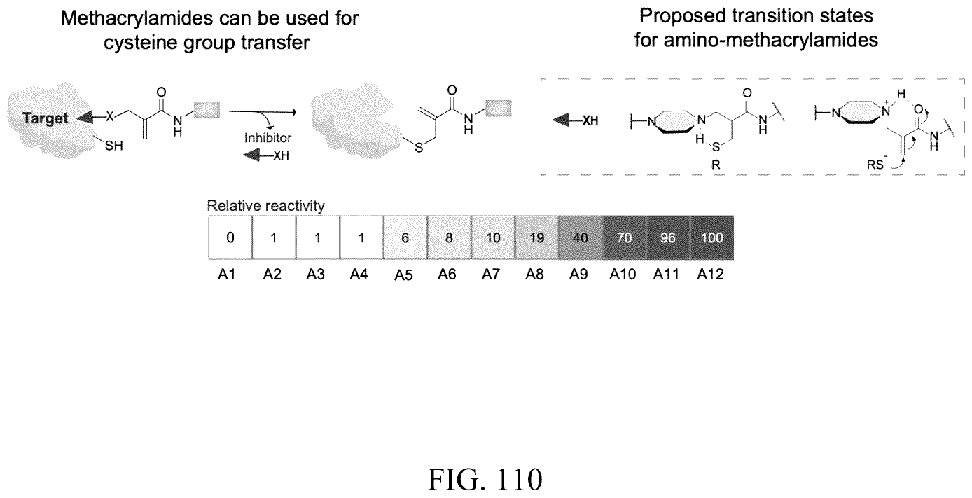

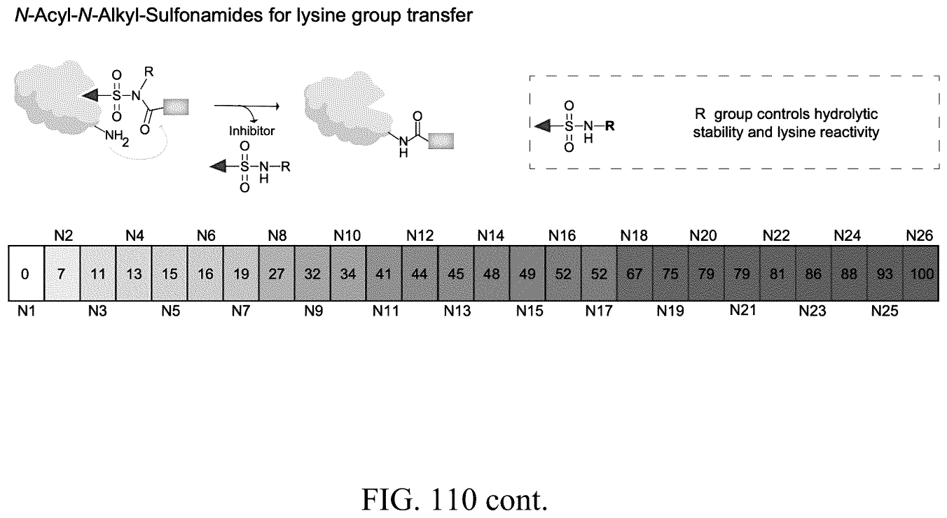

Using kinases, Applicants demonstrated that PWCs can be generated from enzyme inhibitors (see ref 7 for characterization) using an inhibitor of Bruton's Tyrosine Kinase (BTK) (ibrutinib, 2.1, FIG. 96A).9 Here, a nucleophile (e.g., cysteine) near the inhibitor-binding pocket initiates a group transfer, appending the target-protein binder to the kinase while releasing the inhibitor from the active site (FIG. 96A). Applicants connected this BTK inhibitor scaffold to BRD4 binder, (S)-JQ1, via cleavable methacrylamide linker to generate PWC 2.2 (FIG. 96A). To demonstrate this PWC releases the inhibitor from the ATP-binding pocket, Applicants used a reported Bioluminescence Resonance Energy Transfer (BRET) assay 10 wherein inhibitor binding to the ATP pocket reduces the signal (FIG. 96B). Observed BRET ratios demonstrated that while BTK inhibitor 2.1 lowered BRET signal, PWC 2.2 did not (FIG. 96B). Next, Applicants tested the phosphorylation of target BRD4 using PWC 2.2 and inactive analog 2.3 containing (R)-JQ1, a poor binder of BRD4, and expectedly observed more phosphorylation for 2.2 than 2.3 (FIG. 96C).

Applicants next analyzed high-resolution protein:inhibitor co-crystal structures in the Protein Data Bank and found that >98% of inhibitors had Lys within 15 AÃä from the binding pocket, ideal for group transfer. As such, Applicants generated a series of group-transfer linkers with tunable reactivity for Lys (FIG. 96D). Using Cys or Lys group-transfer linkers, Applicants successfully generated PWCs using ˜10 kinases inhibitors (FIG. 96E) of moderate potency (EC₅₀≈1-10 μM in BRET assay).

Lessons Learned from Successful Designs of ˜12 PWCs.

These studies provide design rules for developing PWCs for other PTMs. First, the enzyme binder should have moderate inhibitory potency (≈1 (E°M) so the inhibitor leaves the pocket after labeling. Second, the labeled enzyme can act on multiple target molecules in succession, so the required enzyme labeling efficiencies are low (<5%), which allows the use of low PWC concentrations (e.g., 250 nM of 2.2 in FIG. 96C) that minimize the effect of the released inhibitor. Third, PWCs can append both native and neo-PTMs that are typically not observed in endogenous systems, allowing for a more comprehensive study of cellular function than ever possible. Fourth, the enzyme can be recruited from allosteric or active-site-proximal locations, allowing exploration of multiple neo-interfaces between the enzyme and target. Finally, all PWC editors are cell-permeable small molecules allowing facile dose and temporal control necessary for studying PTMs.

### REFERENCES FOR EXAMPLE 4

(1) The promise and challenge of therapeutic genome editing. Doudna, J. A. Nature 2020, 578, 229-236.PMC8992613
(2) Protein posttranslational modifications: the chemistry of proteome diversifications. Walsh, C. T.; Garneau-Tsodikova, S.; Gatto, G. J., Jr. Angew Chem Int Ed Engl 2005, 44, 7342-72
(3) Post-translational modifications in signal integration. Deribe, Y. L.; Pawson, T.; Dikic, I. Nat Struct Mol Biol 2010, 17, 666-72
(4) Recognizing and exploiting differences between RNAi and small-molecule inhibitors. Weiss, W. A.; Taylor, S. S.; Shokat, K. M. Nat Chem Biol 2007, 3, 739-44.PMC2924165
(5) The Rise of Molecular Glues. Schreiber, S. L. Cell 2021, 184, 3-9
(6) Chemically induced proximity in biology and medicine. Stanton, B. Z.; Chory, E. J.; Crabtree, G. R. Science 2018, 359.PMC6417506
(7) Unifying principles of bifunctional, proximity-inducing small molecules. Gerry, C. J.; Schreiber, S. L. Nat Chem Biol 2020, 16, 369-378.PMC7312755
(8) Bifunctional modalities for repurposing protein function. Modell, A. E.; Lai, S.; Nguyen, T. M.; Choudhary, A. Cell Chem Biol 2021, 28, 1081-1089
(9) Development and applications of chimera platforms for tyrosine phosphorylation. Pergu, R.; Shoba, V. M.; Chaudhary, S. K.; Munkanatta Godage, D. N. P.; Deb, A.; Singha, S.; Dhawa, U.; Anokhina, V.; Singh, S.; Siriwardena, S. U.; Choudhary, A. bioRxiv 2023, 2023.03.05.531183
(10) Quantitative, Wide-Spectrum Kinase Profiling in Live Cells for Assessing the Effect of Cellular ATP on Target Engagement. Vasta, J. D.; Corona, C. R.; Wilkinson, J.; Zimprich, C. A.; Hartnett, J. R.; Ingold, M. R.; Zimmerman, K.; Machleidt, T.; Kirkland, T. A.; Huwiler, K. G.; Ohana, R. F.; Slater, M.; Otto, P.; Cong, M.; Wells, C. I.; Berger, B. T.; Hanke, T.; Glas, C.; Ding, K.; Drewry, D. H.; Huber, K. V. M.; Willson, T. M.; Knapp, S.; Müller, S.; Meisenheimer, P. L.; Fan, F.; Wood, K. V.; Robers, M. B. Cell Chem Biol 2018, 25, 206-214.e11.PMC5814754
(11) Modeling Type 1 Diabetes In Vitro Using Human Pluripotent Stem Cells. Leite, N. C.; Sintov, E.; Meissner, T. B.; Brehm, M. A.; Greiner, D. L.; Harlan, D. M.; Melton, D. A. Cell Rep 2020, 32, 107894.PMC7359783
(12) Neoepitopes in Type 1 Diabetes: Etiological Insights, Biomarkers and Therapeutic Targets. Rodriguez-Calvo, T.; Johnson, J. D.; Overbergh, L.; Dunne, J. L. Front Immunol 2021, 12, 667989.PMC8089389
(13) Type 1 diabetes mellitus. Katsarou, A.; Gudbjörnsdottir, S.; Rawshani, A.; Dabelea, D.; Bonifacio, E.; Anderson, B. J.; Jacobsen, L. M.; Schatz, D. A.; Lernmark, Å. Nat Rev Dis Primers 2017, 3, 17016
(14) Generation of functional human pancreatic β cells in vitro. Pagliuca, F. W.; Millman, J. R.; Gürtler, M.; Segel, M.; Van Dervort, A.; Ryu, J. H.; Peterson, Q. P.; Greiner, D.; Melton, D. A. Cell 2014, 159, 428-39.PMC4617632
(15) A 3D culture platform enables development of zinc-binding prodrugs for targeted proliferation of β cells. Yang, K.; Lee, M.; Jones, P. A.; Liu, S. S.; Zhou, A.; Xu, J.; Sreekanth, V.; Wu, J. L. Y.; Vo, L.; Lee, E. A.; Pop, R.; Lee, Y.; Wagner, B. K.; Melton, D. A.; Choudhary, A.; Karp, J. M. Sci Adv 2020, 6.PMC7673808
(16) A method for the generation of human stem cell-derived alpha cells. Peterson, Q. P.; Veres, A.; Chen, L.; Slama, M. Q.; Kenty, J. H. R.; Hassoun, S.; Brown, M. R.; Dou, H.; Duffy, C. D.; Zhou, Q.; Matveyenko, A. V.; Tyrberg, B.; Sörhede-Winzell, M.; Rorsman, P.; Melton, D. A. Nat Commun 2020, 11, 2241.PMC7205884 described in this manuscript. D. M. is a founder of Semma Therapeutics and equity holder of Semma/Vertex. Semma/Vertex has licensed technology developed in D.M.'s lab. L.C., B.T., and M.S.W. are employees at AstraZeneca. The remaining authors declare no competing interests.
(17) Engineering designer beta cells with a CRISPR-Cas9 conjugation platform. Lim, D.; Sreekanth, V.; Cox, K. J.; Law, B. K.; Wagner, B. K.; Karp, J. M.; Choudhary, A. Nat Commun 2020, 11, 4043.PMC7426819
(18) Native Zinc Catalyzes Selective and Traceless Release of Small Molecules in β-Cells. Lee, M.; Maji, B.; Manna, D.; Kahraman, S.; Elgamal, R. M.; Small, J.; Kokkonda, P.; Vetere, A.; Goldberg, J. M.; Lippard, S. J.; Kulkarni, R. N.; Wagner, B. K.; Choudhary, A. J Am Chem Soc 2020, 142, 6477-6482.PMC7146867 Institute has filed PCT/US2018/028660 that claims inventions related to targeted delivery to beta cells.
(19) Harnessing reaction-based probes to preferentially target pancreatic B-cells and β-like cells. Kahraman, S.; Manna, D.; Dirice, E.; Maji, B.; Small, J.; Wagner, B. K.; Choudhary, A.; Kulkarni, R. N. Life Sci Alliance 2021, 4.PMC7898467
(20) Fluorescein-based sensors to purify human α-cells for functional and transcriptomic analyses. Kahraman, S.; Shibue, K.; De Jesus, D. F.; Hu, J.; Manna, D.; Wagner, B. K.; Choudhary, A.; Kulkarni, R. N. bioRxiv 2022, 2022.11.27.518097
(21) Phosphorylation-Inducing Chimeric Small Molecules. Siriwardena, S. U.; Munkanatta Godage, D. N. P.; Shoba, V. M.; Lai, S.; Shi, M.; Wu, P.; Chaudhary, S. K.; Schreiber, S. L.; Choudhary, A. J Am Chem Soc 2020, 142, 14052-14057
(22) Synthetic Reprogramming of Kinases Expands Cellular Activities of Proteins. Shoba, V. M.; Munkanatta Godage, D. N. P.; Chaudhary, S. K.; Deb, A.; Siriwardena, S. U.; Choudhary, A. Angew Chem Int Ed Engl 2022, 61, e202202770.PMC9527066
(23) Citrullination of glucokinase is linked to autoimmune diabetes. Yang, M. L.; Horstman, S.; Gee, R.; Guyer, P.; Lam, T. T.; Kanyo, J.; Perdigoto, A. L.; Speake, C.; Greenbaum, C. J.; Callebaut, A.; Overbergh, L.; Kibbey, R. G.; Herold, K. C.; James, E. A.; Mamula, M. J. Nat Commun 2022, 13, 1870.PMC8986778
(24) Recognition of posttranslationally modified GAD65 epitopes in subjects with type 1 diabetes. McGinty, J. W.; Chow, I. T.; Greenbaum, C.; Odegard, J.; Kwok, W. W.; James, E. A. Diabetes 2014, 63, 3033-40.PMC4392921
(25) Chromogranin A is an autoantigen in type 1 diabetes. Stadinski, B. D.; Delong, T.; Reisdorph, N.; Reisdorph, R.; Powell, R. L.; Armstrong, M.; Piganelli, J. D.; Barbour, G.; Bradley, B.; Crawford, F.; Marrack, P.; Mahata, S. K.; Kappler, J. W.; Haskins, K. Nat Immunol 2010, 11, 225-31.PMC3166626
(26) How C-terminal additions to insulin B-chain fragments create superagonists for T cells in mouse and human type 1 diabetes. Wang, Y.; Sosinowski, T.; Novikov, A.; Crawford, F.; White, J.; Jin, N.; Liu, Z.; Zou, J.; Neau, D.; Davidson, H. W.; Nakayama, M.; Kwok, W. W.; Gapin, L.; Marrack, P.; Kappler, J. W.; Dai, S. Sci Immunol 2019, 4.PMC6929690
(27) Lysosomal cathepsin creates chimeric epitopes for diabetogenic CD4 T cells via transpeptidation. Reed, B.; Crawford, F.; Hill, R. C.; Jin, N.; White, J.; Krovi, S. H.; Marrack, P.; Hansen, K.; Kappler, J. W. J Exp Med 2021, 218.PMC7590512 submitted work. No other disclosures were reported.
(28) Identification of a common autoantigenic epitope of protein disulfide isomerase, golgin-160 and voltage-gated potassium channel in type 1 diabetes. Fierabracci, A.; Saura, F. Diabetes Res Clin Pract 2010, 88, e14-6
(29) Cellular death, reactive oxygen species (ROS) and diabetic complications. Volpe, C. M. O.; Villar-Delfino, P. H.; Dos Anjos, P. M. F.; Nogueira-Machado, J. A. Cell Death Dis 2018, 9, 119.PMC5833737
(30) Nitric oxide metabolism is impaired by type 1 diabetes and diabetic nephropathy. Sokolovska, J.; Dekante, A.; Baumane, L.; Pahirko, L.; Valeinis, J.; Dislere, K.; Rovite, V.; Pirags, V.; Sjakste, N. Biomed Rep 2020, 12, 251-258.PMC7100134
(31) Citrullination and PAD Enzyme Biology in Type 1 Diabetes-Regulators of Inflammation, Autoimmunity, and Pathology. Yang, M. L.; Sodré, F. M. C.; Mamula, M. J.; Overbergh, L. Front Immunol 2021, 12, 678953.PMC8204103
(32) The interplay between citrullination and HLA-DRB1 polymorphism in shaping peptide binding hierarchies in rheumatoid arthritis. Ting, Y. T.; Petersen, J.; Ramarathinam, S. H.; Scally, S. W.; Loh, K. L.; Thomas, R.; Suri, A.; Baker, D. G.; Purcell, A. W.; Reid, H. H.; Rossjohn, J. J Biol Chem 2018, 293, 3236-3251.PMC5836122
(33) Autoantigenesis: the evolution of protein modifications in autoimmune disease. Doyle, H. A.; Mamula, M. J. Curr Opin Immunol 2012, 24, 112-8.PMC3288476
(34) The clinical significance of posttranslational modification of autoantigens. Zavala-Cerna, M. G.; Martínez-García, E. A.; Torres-Bugarín, O.; Rubio-Jurado, B.; Riebeling, C.; Nava, A. Clin Rev Allergy Immunol 2014, 47, 73-90
(35) Inhibition of PAD4 activity is sufficient to disrupt mouse and human NET formation. Lewis, H. D.; Liddle, J.; Coote, J. E.; Atkinson, S. J.; Barker, M. D.; Bax, B. D.; Bicker, K. L.; Bingham, R. P.; Campbell, M.; Chen, Y. H. Nature chemical biology 2015, 11, 189-191
(36) Irreversible 4-aminopiperidine transglutaminase 2 inhibitors for Huntington's disease. Prime, M. E.; Brookfield, F. A.; Courtney, S. M.; Gaines, S.; Marston, R. W.; Ichihara, O.; Li, M.; Vaidya, D.; Williams, H.; Pedret-Dunn, A. ACS Med Chem Lett 2012, 3, 731-735
(37) Discovery and structure-activity relationship of potent and selective covalent inhibitors of transglutaminase 2 for Huntington's disease. Prime, M. E.; Andersen, O. A.; Barker, J. J.; Brooks, M. A.; Cheng, R. K.; Toogood-Johnson, I.; Courtney, S. M.; Brookfield, F. A.; Yarnold, C. J.; Marston, R. W. J Med Chem 2012, 55, 1021-1046
(38) A randomized trial of a transglutaminase 2 inhibitor for celiac disease. Schuppan, D.; Mäki, M.; Lundin, K. E.; Isola, J.; Friesing-Sosnik, T.; Taavela, J.; Popp, A.; Koskenpato, J.; Langhorst, J.; Hovde, Ø. NEJM 2021, 385, 35-45
(39) Features of ZED1227: The first-in-class tissue transglutaminase inhibitor undergoing clinical evaluation for the treatment of celiac disease. Büchold, C.; Hils, M.; Gerlach, U.; Weber, J.; Pelzer, C.; Heil, A.; Aeschlimann, D.; Pasternack, R. Cells 2022, 11, 1667
(40) Dipeptidyl nitrile inhibitors of Cathepsin L. Asaad, N.; Bethel, P. A.; Coulson, M. D.; Dawson, J. E.; Ford, S. J.; Gerhardt, S.; Grist, M.; Hamlin, G. A.; James, M. J.; Jones, E. V. Bioorg Medl Chem Lett 2009, 19, 4280-4283
(41) Discovery of an orally active small-molecule irreversible inhibitor of protein disulfide isomerase for ovarian cancer treatment. Xu, S.; Butkevich, A. N.; Yamada, R.; Zhou, Y.; Debnath, B.; Duncan, R.; Zandi, E.; Petasis, N. A.; Neamati, N. Proceedings of the National Academy of Sciences 2012, 109, 16348-16353
(42) First contact: 7-phenyl-2-aminoquinolines, potent and selective neuronal nitric oxide synthase inhibitors that target an isoform-specific aspartate. Cinelli, M. A.; Reidl, C. T.; Li, H.; Chreifi, G.; Poulos, T. L.; Silverman, R. B. J Med Chem 2020, 63, 4528-4554
(43) Chemogenetic System Demonstrates That Cas9 Longevity Impacts Genome Editing Outcomes. Sreekanth, V.; Zhou, Q.; Kokkonda, P.; Bermudez-Cabrera, H. C.; Lim, D.; Law, B. K.; Holmes, B. R.; Chaudhary, S. K.; Pergu, R.; Leger, B. S.; Walker, J. A.; Gifford, D. K.; Sherwood, R. I.; Choudhary, A. ACS Cent Sci 2020, 6, 2228-2237.PMC7760466
(44) Template-assisted covalent modification of DCAF16 underlies activity of BRD4 molecular glue degraders. Li, Y.-D.; Ma, M. W.; Hassan, M. M.; Hunkeler, M.; Teng, M.; Puvar, K.; Lumpkin, R.; Sandoval, B.; Jin, C. Y.; Ficarro, S. B.; Wang, M. Y.; Xu, S.; Groendyke, B. J.; Sigua, L. H.; Tavares, I.; Zou, C.; Tsai, J. M.; Park, P. M. C.; Yoon, H.; Majewski, F. C.; Marto, J. A.; Qi, J.; Nowak, R. P.; Donovan, K. A.; Słabicki, M.; Gray, N. S.; Fischer, E. S.; Ebert, B. L. bioRxiv 2023, 2023.02.14.528208
(45) Search-and-replace genome editing without double-strand breaks or donor DNA. Anzalone, A. V.; Randolph, P. B.; Davis, J. R.; Sousa, A. A.; Koblan, L. W.; Levy, J. M.; Chen, P. J.; Wilson, C.; Newby, G. A.; Raguram, A.; Liu, D. R. Nature 2019, 576, 149-157.PMC6907074
(46) Induction of pluripotent stem cells from mouse embryonic and adult fibroblast cultures by defined factors. Takahashi, K.; Yamanaka, S. Cell 2006, 126, 663-76
(47) The spliceosomal phosphopeptide P140 controls the lupus disease by interacting with the HSC70 protein and via a mechanism mediated by gammadelta T cells. Page, N.; Schall, N.; Strub, J. M.; Quinternet, M.; Chaloin, O.; Décossas, M.; Cung, M. T.; Van Dorsselaer, A.; Briand, J. P.; Muller, S. PLOS One 2009, 4, e5273.PMC2669294
(48) Nitrosative Stress, Hypernitrosylation, and Autoimmune Responses to Nitrosylated Proteins: New Pathways in Neuroprogressive Disorders Including Depression and Chronic Fatigue Syndrome. Morris, G.; Berk, M.; Klein, H.; Walder, K.; Galecki, P.; Maes, M. Mol Neurobiol 2017, 54, 4271-4291
(49) A covalent inhibitor of K-Ras (G12C) induces MHC class I presentation of haptenated peptide neoepitopes targetable by immunotherapy. Zhang, Z.; Rohweder, P. J.; Ongpipattanakul, C.; Basu, K.; Bohn, M. F.; Dugan, E. J.; Steri, V.; Hann, B.; Shokat, K. M.; Craik, C. S. Cancer Cell 2022, 40, 1060-1069.e7
(50) Advances in covalent kinase inhibitors. Abdeldayem, A.; Raouf, Y. S.; Constantinescu, S. N.; Moriggl, R.; Gunning, P. T. Chem Soc Rev 2020, 49, 2617-2687
(51) RAS-targeted therapies: is the undruggable drugged? Moore, A. R.; Rosenberg, S. C.; McCormick, F.; Malek, S. Nat Rev Drug Discov 2020, 19, 533-552
(52) KRAS (G12D) can be targeted by potent inhibitors via formation of salt bridge. Mao, Z.; Xiao, H.; Shen, P.; Yang, Y.; Xue, J.; Yang, Y.; Shang, Y.; Zhang, L.; Li, X.; Zhang, Y.; Du, Y.; Chen, C. C.; Guo, R. T.; Zhang, Y. Cell Discov 2022, 8, 5.PMC8786924
(53) Discovering new biology with drug-resistance alleles. Freedy, A. M.; Liau, B. B. Nat Chem Biol 2021, 17, 1219-1229.PMC9530778
(54) CRISPR-Suppressor Scanning for Systematic Discovery of Drug-Resistance Mutations. Ngan, K. C.; Lue, N. Z.; Lee, C.; Liau, B. B. Curr Protoc 2022, 2, e614
(55) The dynamic clustering of insulin receptor underlies its signaling and is disrupted in insulin resistance. Dall′Agnese, A.; Platt, J. M.; Zheng, M. M.; Friesen, M.; Dall'Agnese, G.; Blaise, A. M.; Spinelli, J. B.; Henninger, J. E.; Tevonian, E. N.; Hannett, N. M.; Lazaris, C.; Drescher, H. K.; Bartsch, L. M.; Kilgore, H. R.; Jaenisch, R.; Griffith, L. G.; Cisse, II; Jeppesen, J. F.; Lee, T. I.; Young, R. A. Nat Commun 2022, 13, 7522.PMC9727033
(56) Biomolecular condensates in membrane receptor signaling. Jaqaman, K.; Ditlev, J. A. Curr Opin Cell Biol 2021, 69, 48-54.PMC8058224
(57) Mechanisms of disease: Molecular and metabolic mechanisms of insulin resistance and beta-cell failure in type 2 diabetes. Muoio, D. M.; Newgard, C. B. Nat Rev Mol Cell Biol 2008, 9, 193-205
(58) Mechanisms of Insulin Action and Insulin Resistance. Petersen, M. C.; Shulman, G. I. Physiol Rev 2018, 98, 2133-2223.PMC6170977
(59) A Membrane-Bound Diacylglycerol Species Induces PKCε-Mediated Hepatic Insulin Resistance. Lyu, K.; Zhang, Y.; Zhang, D.; Kahn, M.; Ter Horst, K. W.; Rodrigues, M. R. S.; Gaspar, R. C.; Hirabara, S. M.; Luukkonen, P. K.; Lee, S.; Bhanot, S.; Rinehart, J.; Blume, N.; Rasch, M. G.; Serlie, M. J.; Bogan, J. S.; Cline, G. W.; Samuel, V. T.; Shulman, G. I. Cell Metab 2020, 32, 654-664.e5.PMC7544641
(60) Serine/threonine phosphorylation of IRS-1 triggers its degradation: possible regulation by tyrosine phosphorylation. Pederson, T. M.; Kramer, D. L.; Rondinone, C. M. Diabetes 2001, 50, 24-31
(61) Regulation of insulin sensitivity by serine/threonine phosphorylation of insulin receptor substrate proteins IRS1 and IRS2. Copps, K. D.; White, M. F. Diabetologia 2012, 55, 2565-2582.PMC4011499
(62) Insulin resistance: Review of the underlying molecular mechanisms. Yaribeygi, H.; Farrokhi, F. R.; Butler, A. E.; Sahebkar, A. J Cell Physiol 2019, 234, 8152-8161
(63) Phase separation enhances probability of receptor signalling and drug targeting. Ladbury, J. E.; Lin, C. C.; Suen, K. M. Trends Biochem Sci 2023
(64) Diabetes comorbidities in low- and middle-income countries: An umbrella review. Lam, A. A.; Lepe, A.; Wild, S. H.; Jackson, C. J Glob Health 2021, 11, 04040.PMC8325931
(65) Luciferase Reporter Assay System for Deciphering GPCR Pathways. Cheng, Z.; Garvin, D.; Paguio, A.; Stecha, P.; Wood, K.; Fan, F. Curr Chem Genomics 2010, 4, 84-91.PMC3040460
(66) PKC-phosphorylation of Liprin-α3 triggers phase separation and controls presynaptic active zone structure. Emperador-Melero, J.; Wong, M. Y.; Wang, S. S. H.; de Nola, G.; Nyitrai, H.; Kirchhausen, T.; Kaeser, P. S. Nat Commun 2021, 12, 3057.PMC8144191
(67) Small-molecule control of insulin and PDGF receptor signaling and the role of membrane attachment. Yang, J.; Symes, K.; Mercola, M.; Schreiber, S. L. Curr Biol 1998, 8, 11-8

### Example 5—Lysine-Based Group Transfer Chemistry for Protein Editing

CRISPR-based technologies are revolutionizing biomedicine by making facile modifications of DNA/RNA. However, technologies to systematically “write” or “erase” post-translational modifications (PTMs) on a protein of interest (POI) are still in their infancy. One approach to PTM editing involves using chimeric molecules of POI and PTM-writing/erasing enzymes. The traditional design of chimeric molecules limits their applications, as attracting a fully functioning enzyme requires the use of high-affinity, allosteric, and non-inhibitory binders that are scarce. Discovery of non-inhibitory, allosteric binders can be resource and time-intensive, and identifying non-inhibitory pockets on enzymes is non-trivial or may not even exist.

On the contrary, most PTM-writing/erasing enzymes have inhibitors. Applicants envisioned a design of chimeric molecules based on group transfer in which the inhibitor facilitates proximity-driven covalent labeling of the enzyme with POI binder by targeting lysine with concomitant release of the inhibitor. The released non-covalent inhibitor has a reduced affinity that, combined with the highly abundant substrate (e.g., ATP for kinase), displaces from the active site to allow normal enzyme function.

Most lysine-targeting group transfer linkers suffer from poor reaction rates, lack of stability/selectivity, and a bulky nature, which render them unsuitable for designing chimeric molecules. N-acyl-N-alkyl sulfonamides (NASA) have recently gained interest as covalent warheads for lysine targeting due to their fast reaction rates towards lysine (k≈104 M⁻¹ s⁻¹). Despite these promising attributes, NASA is bulky, hydrolyzes rapidly (t½≈5 h), and lacks analogs with tunable reactivity. Applicants have developed ≈25 group transfer linkers with a smaller footprint, higher stability, and tunable reactivity. Using these linkers, Applicants have developed chimeras that can induce or remove several post-translational modifications on diverse protein targets, starting with abundantly available inhibitors of PTM writer or eraser enzymes. These results establish design principles to generate PTM writing or erasing chimeras starting with enzyme inhibitors and set the stage for a universal platform for PTM editing on proteins of interest mediated by group transfer chemistry.

Proximity-inducing heterobifunctional modalities traditionally have been limited to tag-based systems or allosteric binders. Applicants have successfully developed a group transfer approach that repurposes an inhibitor into a directing group. The bioinformatic analysis of PDB crystal structures suggests that most PTM enzymes have targetable proximal lysine residues, confirming that lysine is a suitable target to expand the platform to various PTMs. Among the different lysine-targeting warheads, NASA offers fast reaction rates and the ability to tune stability and reactivity. Applicants have rationally tuned the NASA warhead to increase stability while retaining lysine reactivity. Applicants utilized these optimized NASA warheads to generate the first-in-class lysine targeting PHICs molecules by recruiting BTK and EphA2 to phosphorylate the proof-of-concept target BRD4. LC-MS/MS analysis confirmed the labeling of lysine residues as expected based on the structure-based design of the PHICS molecules. The establishment of the lysine targeting platform will enable Applicants to expand the group transfer approach to other PTMs.

### Example 6—Cysteine-Based Group Transfer Chimeras to Edit Post-Translational Modifications

Applicants have developed a platform that leverages the advantages of covalency for specificity while capitalizing on the weaknesses of non-covalency, such as a shorter residence time and moderate affinity. Applicants have utilized highly abundant and optimized inhibitors for designing PTM editors for inducing PTM using cystine-based group transfer chemistry. The insights gained from this study has allowed us to establish general design principles for PTM editors, which include key properties such as desired cysteine proximity, reactivity level, residence time and affinity of the released non-covalent inhibitor, and functional kinase activity in the presence of conjugated protein binder. The in-silico studies suggest that using this approach allows us to target over 50% of kinases and has the potential to be extended to include nine additional PTM editors. Utilizing this platform, Applicants have developed PTM editors using FGFR inhibitors to activate the FGFR signaling pathway. This opens avenues for creating growth factor mimetics for activation of signaling pathways, holding significance in clinical, industrial, or environmental contexts.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An electrophilic reactive linker according to the formula L₁-El, El-L₁, or Li-El-L₂, wherein El is an electrophilic reactive group and wherein L₁ and L₂ are linking molecules.

2. The electrophilic reactive linker of claim 1, wherein the El reacts with a nucleophilic reactive group.

3. The electrophilic reactive linker of claim 1, wherein El is configured to facilitate attachment of the electrophilic reactive group and all or a portion of L₁ or L₂ to a Cysteine, Serine, Threonine, Tyrosine, Glutamic Acid, Aspartic Acid, Lysine, Arginine, Histidine, or a Methionine amino acid on a polypeptide.

4. The electrophilic reactive linker of claim 1, wherein the linking molecules L₁ and L₂ are independently selected from alkane, alkene, amine, either, thiol, sulfone, carbonyl, acyl, ketone, carboxylate ester, amide, enone, anhydride, imide, PEG, or any combination thereof.

5. The electrophilic reactive linker of claim 1, wherein the linking molecules L₁ and/or L₂ comprise rigid molecules.

6. The electrophilic reactive linker of claim 1, wherein the electrophilic reactive group is selected from N-acyl-N-alkyl sulfonamide (NASA), dibromophenyl benzoate, or N-sulfonyl pyridone.

7. The electrophilic reactive linker of claim 1, wherein the El is a photo-reactive group.

8. The electrophilic reactive linker of claim 1, wherein the electrophilic reactive group is selected from the group consisting of:

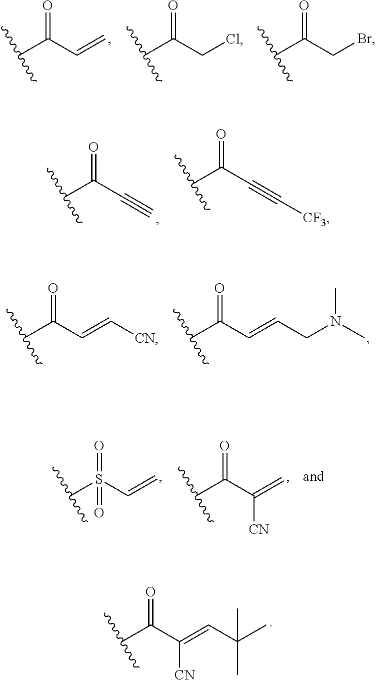

9. The electrophilic reactive linker of claim 1, wherein the electrophilic reactive group has the formula: 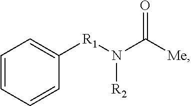
wherein R₁ is selected from C—O, SO₂, Me-C—O, or Me-SO₂, R₂ is selected from H, alkane, alkene, alkyne, amine, nitrile, nitro, ether, alcohol, thiol, sulfone, sulfonate, halogen, carbonyl; acyl; ketone; carboxylate ester; amide; enone; anhydride; imide, cyclic hydrocarbon, an unsaturated cyclic hydrocarbon, a heterocycle, one or more fused rings thereof; an aliphatic halide such as —OCF₂Cl or any combination thereof, and the benzene ring is optionally substituted at any position.

10. The electrophilic reactive linker of claim 1, wherein the electrophilic reactive group is selected from the group consisting of:

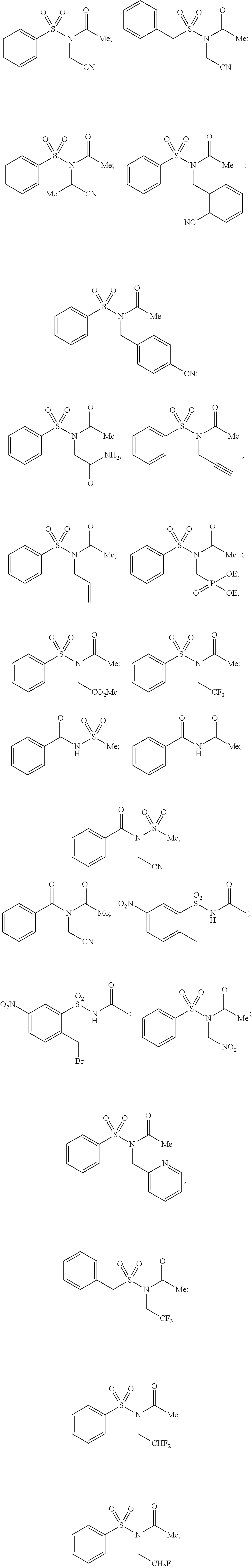

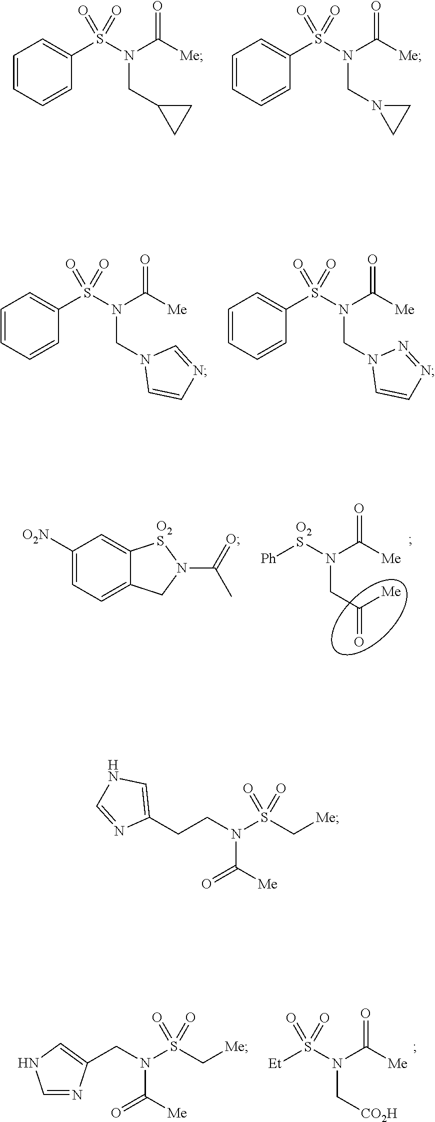

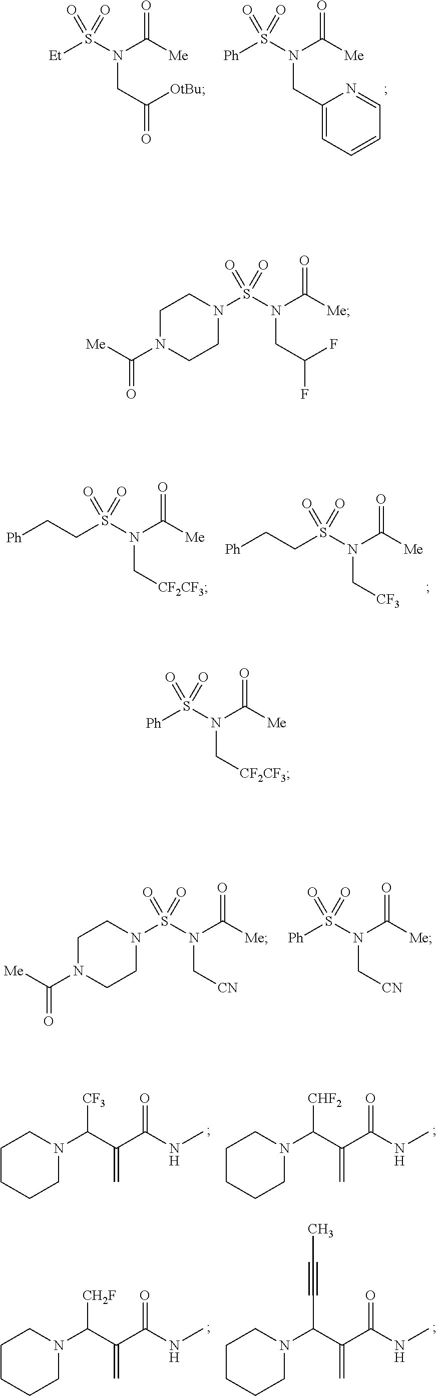

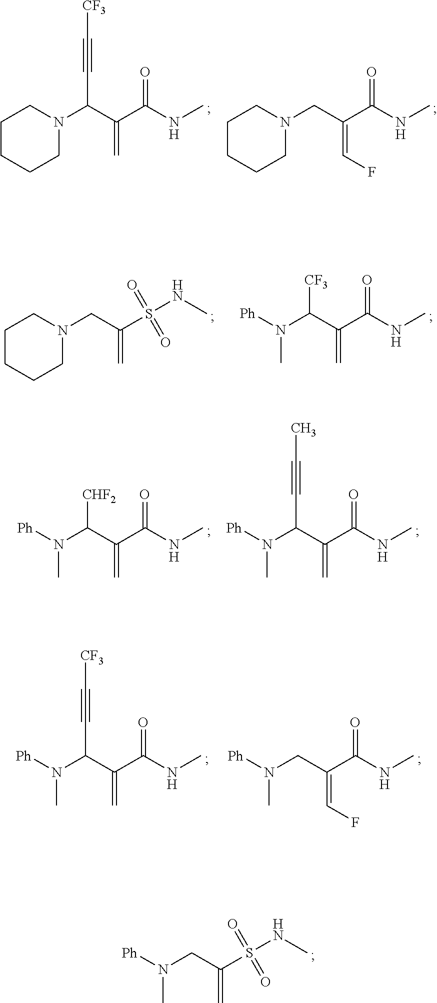

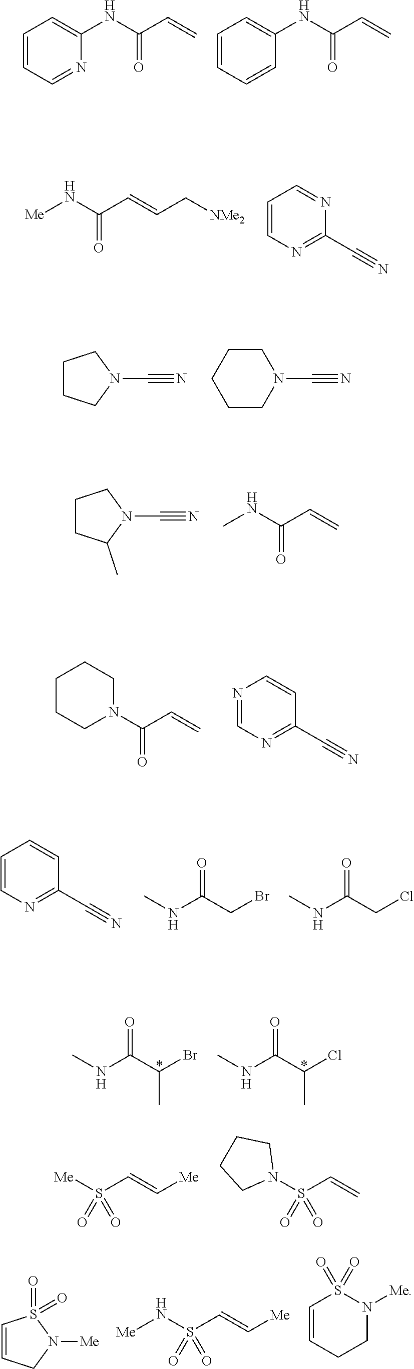

11. The electrophilic reactive linker of claim 1, wherein the El is configured to facilitate attachment of all or a portion of L₁ or L₂ to a cysteine, and wherein the El is selected from the group consisting of:

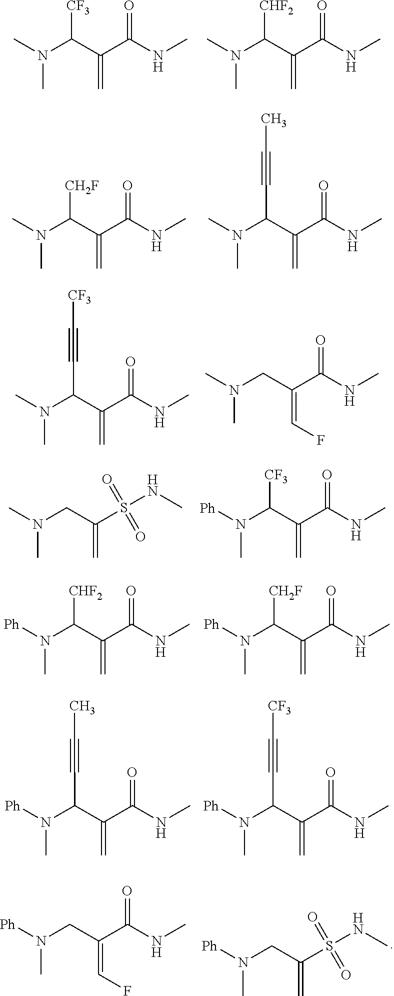

12. The electrophilic reactive linker of claim 1, wherein the El is configured to facilitate attachment of all or a portion of L₁ or L₂ to a lysine, and wherein the El is 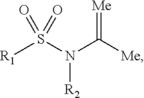
wherein R₁ is selected from the group consisting of: 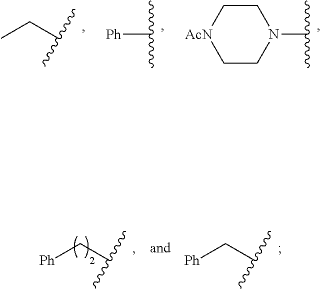
and
wherein R₂ is selected from the group consisting of:

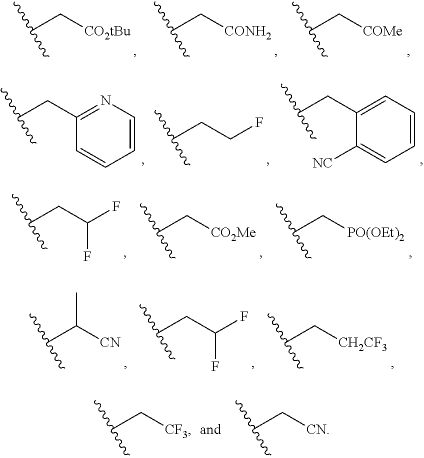

13. The electrophilic reactive linker of claim 1, wherein the El is configured to facilitate attachment of all or a portion of L₁ or L₂ to a lysine, and wherein the El is selected from the group consisting of:

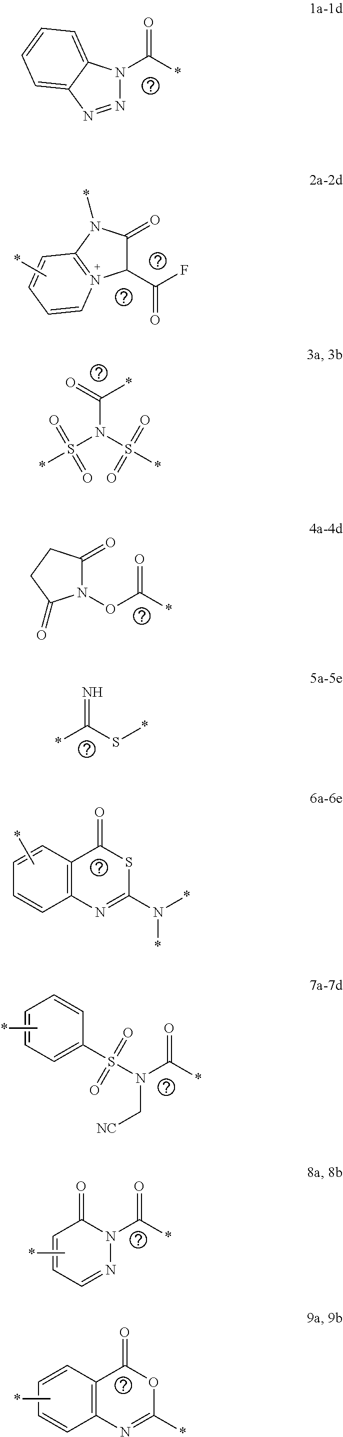

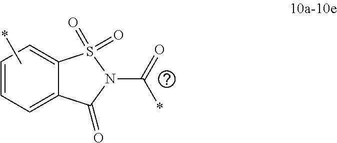

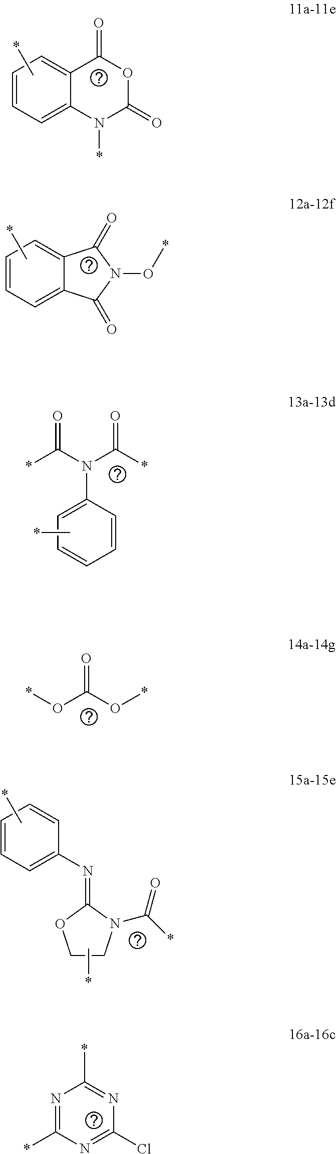

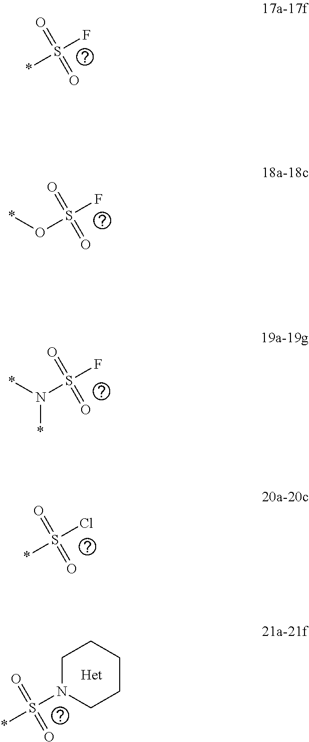 [SMILES: *S(=O)(=O)F ; *OS(=O)(=O)F ; *N(*)S(=O)(=O)F ; *S(=O)(=O)Cl ; *S(=O)(=O)N1CCCCC1]

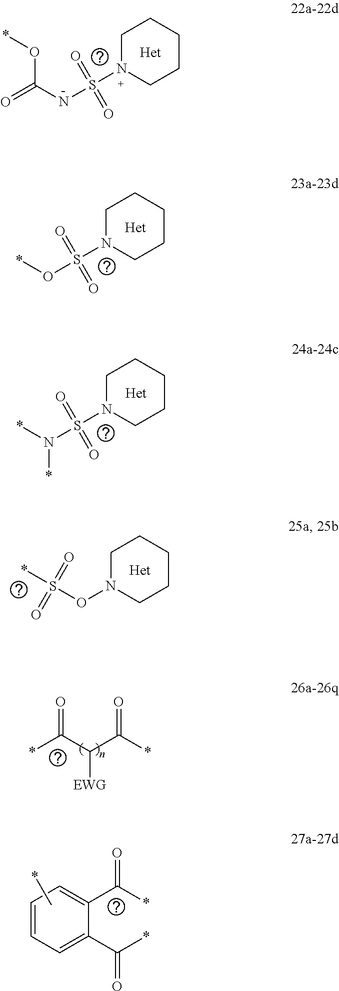

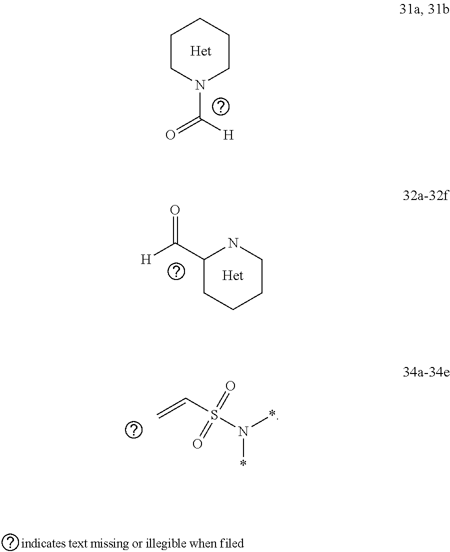

14. The electrophilic reactive linker of claim 1, wherein the El is configured to facilitate attachment of all or a portion of L₁ or L₂ to a methionine, and wherein the El is selected from the group consisting of:

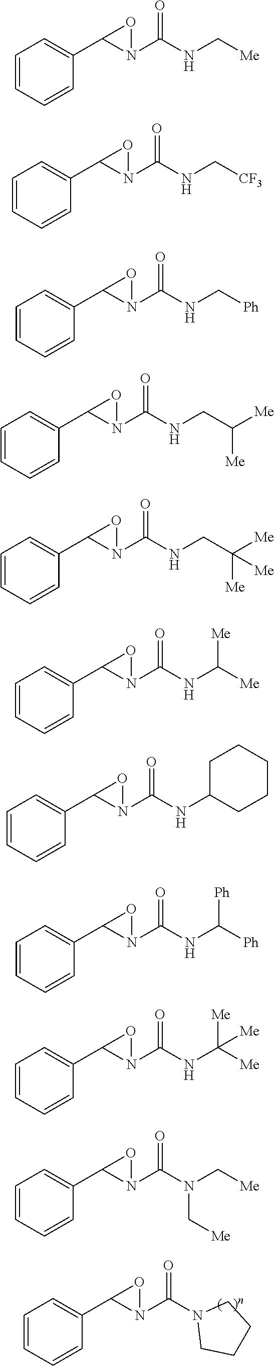

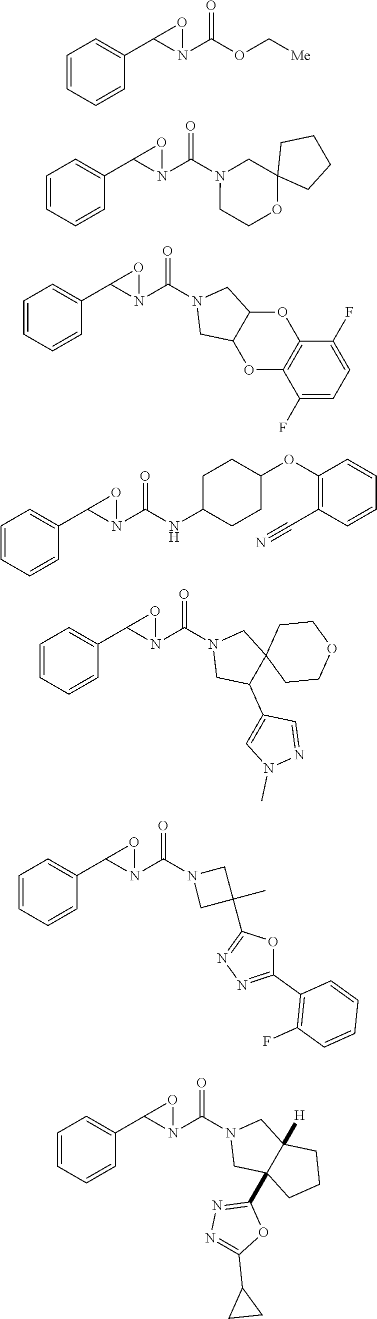

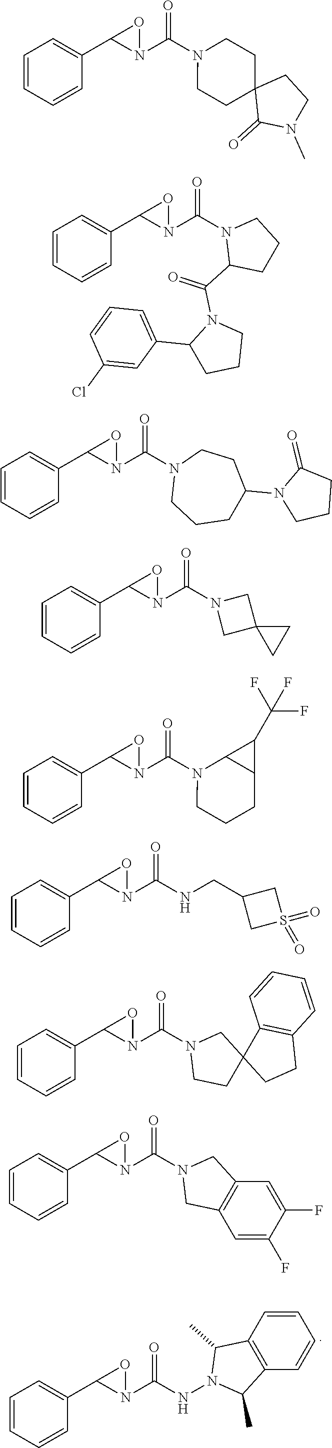

15. The electrophilic reactive linker of claim 1, wherein the electrophilic reactive group is 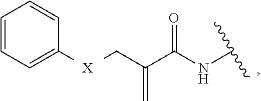
wherein X is selected from the group consisting of: 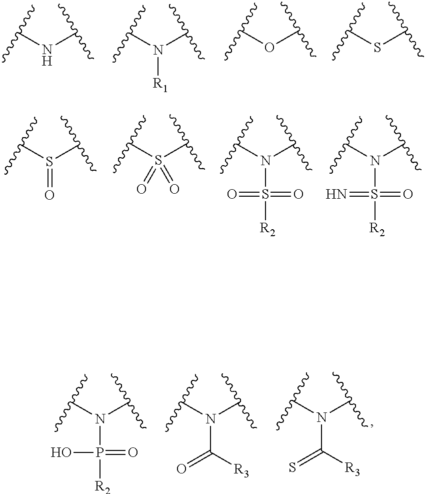
wherein R₁, R2, R3 individually comprise of an alkyl group, aryl group, or a heteroatom optionally O, N, or S.

16. The electrophilic reactive linker of claim 1, wherein the electrophilic reactive group is 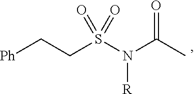
wherein R is selected from the group consisting of:

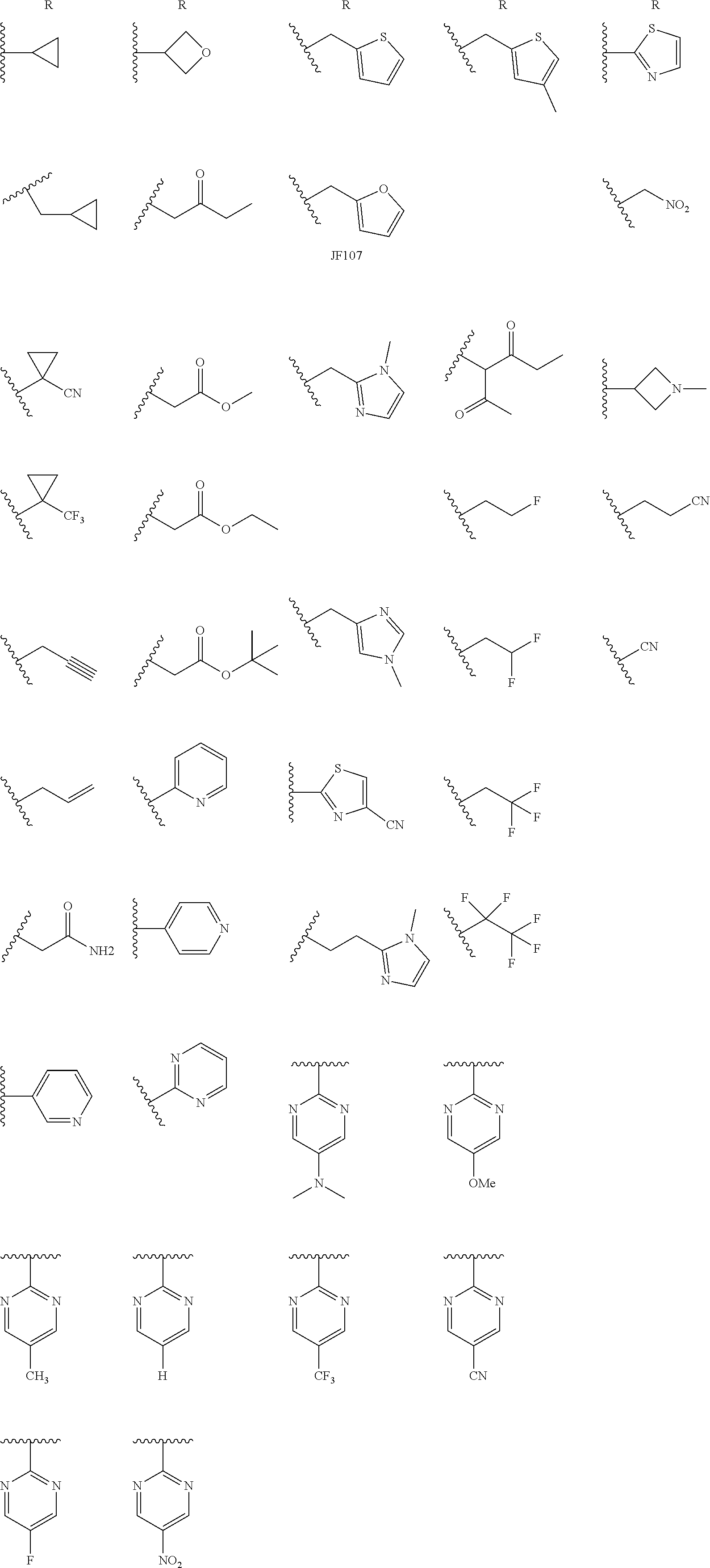

17. The electrophilic reactive linker of claim 1, wherein the electrophilic reactive group is 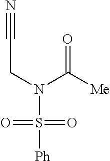 [SMILES: CC(=O)N(CC#N)S(=O)(=O)c1ccccc1]
or an analog thereof.

18. The electrophilic reactive linker of claim 1, wherein the electrophilic reactive group is 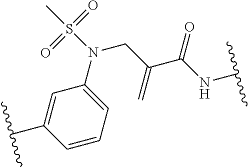 [SMILES: *NC(=O)C(=C)CN(c1cccc(*)c1)S(C)(=O)=O]
or an analog thereof.

19. The electrophilic reactive linker of claim 1, wherein the electrophilic reactive group is 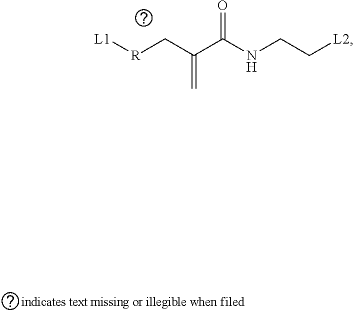
wherein R is selected from the group consisting of:

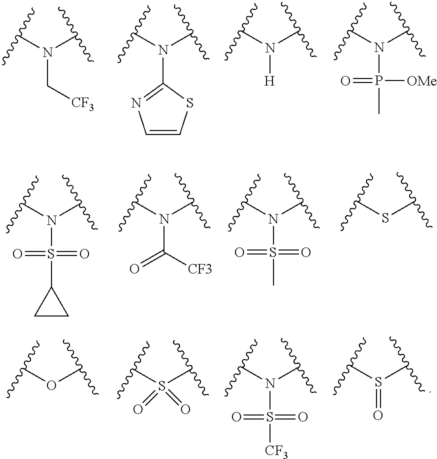

20. The electrophilic reactive linker of claim 1, wherein the electrophilic reactive group linker further comprises a bio-orthogonal group,
optionally wherein the bio-orthogonal group is selected from tetrazines, triazines, cyclooctenes, cyclopropenes and diazo, or
wherein the bio-orthogonal group is selected from the group consisting of:

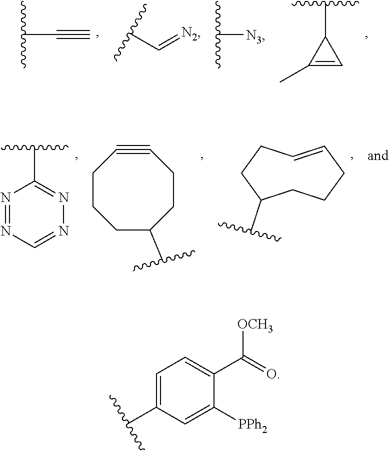

21-22. (canceled)

23. The electrophilic reactive linker of claim 1, wherein the electrophilic reactive group linker further comprises an orienting adaptor, optionally wherein the orienting adaptor is selected from the group consisting of:

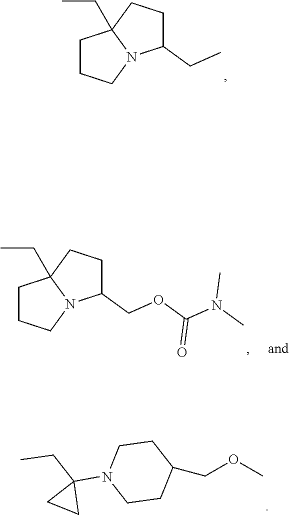

24. (canceled)

25. The electrophilic reactive linker of claim 1, wherein the electrophilic reactive linker is capable of covalently labeling a target polypeptide, optionally wherein labeling comprises covalently bonding to a nucleophile disposed on the target polypeptide.

26. (canceled)

27. The electrophilic reactive linker of claim 1, further comprising a target binding moiety connected to L₁ or L₂ and configured to allow the El to covalently attach to the target polypeptide and/or further comprising a modifying polypeptide binding moiety located on a side of L₁ or L₂ opposite the El and capable of binding a modifying polypeptide.

28. (canceled)

29. The electrophilic reactive linker of claim 1, wherein the El reversibly bonds to the target polypeptide.

30. The electrophilic reactive linker of claim 1, wherein the modifying polypeptide is a phosphatase, a ubiquitinase, a deubiquitinase, an acetyltransferase, deactylase, methyltransferase, demethylase, or glycosyltransferase and the modifying polypeptide binding moiety binds a neo-substrate for the phosphatase, the ubiquitinase, the deubiquitinase, the acetyltransferase, the deactylase, the methyltransferase, the demethylase, or the glycosyltransferase.

31. A bifunctional molecule comprising the electrophilic reactive linker of claim 1 and a target polypeptide binding moiety attached to the linker on one end and a modifying polypeptide binding moiety attached to the linker on an opposite end.

32. The bifunctional molecule of claim 0, wherein the modifying polypeptide binding moiety binds to a phosphatase, a ubiquitinase, a deubiquitinase, an acetyltransferase, deactylase, methyltransferase, demethylase, or glycosyltransferase and wherein the target polypeptide binding moiety binds to a polypeptide to which the modifying moiety is to be attached and modified by the modifying moiety,
optionally wherein the electrophilic reactive linker connects the functional moieties of a proteolysis targeting chimeras (PROTAC), deubiquitinase-targeting chimeras (DUBTACs), Pho-repressive complex (PhoRC), dephosphorylation targeting chimera (DEPTAC), phosphorylation targeting chimeras (PhosTAC), acetylation tagging system (AceTAG), regulated induced proximity targeting chimeras (RIPTAC), Transcriptional/Epigenetic Chemical Inducers of Proximity (TCIP), Autophagy-Targeting Chimera (AUTAC), Lysosome Targeting Chimeras (LYTAC), and or immune cell recruiting chimeras.

33-34. (canceled)
